(19) United States
(12) Patent Application Publication
(10) Pub. No.: US 2024/0174732 A1
(43) Pub. Date: May 30, 2024

(54) NUCLEIC ACID MOLECULES ENCODING TRIF AND ADDITIONAL POLYPEPTIDES AND THEIR USE IN TREATING CANCER

(71) Applicant: Flagship Pioneering Innovations V, Inc., Cambridge, MA (US)

(72) Inventors: Benjamin C. Blum, Wayland, MA (US); Alexis Benoit Hubaud, Somerville, MA (US); Peter Joseph Gough, Wayne, PA (US); Sabin Dhakal, Brookline, MA (US); Shreyas Jogidas Jadhav, Belmont, MA (US); Lauren Jessica Lahey, Brookline, MA (US); Erik Christian Hett, Arlington, MA (US); Christopher Ryan Shaler, Cambridge, MA (US); Loren Matthew Brown, Cambridge, MA (US); Xuqing Zhang, Quincy, MA (US); Yeon Sook Choi, Holliston, MA (US)

(21) Appl. No.: 18/481,993

(22) Filed: Oct. 5, 2023

Related U.S. Application Data
(60) Provisional application No. 63/463,850, filed on May 3, 2023.
(60) Provisional application No. 63/437,982, filed on Jan. 9, 2023.
(60) Provisional application No. 63/413,566, filed on Oct. 5, 2022.

(51) Int. Cl. C07K 14/705; A61K 31/7105; A61P 35/00; C07K 14/47; C07K 14/54; C12N 9/12

(52) U.S. Cl. CPC C07K 14/70596; A61K 31/7105; A61P 35/00; C07K 14/4747; C07K 14/5434; C12N 9/12; C12Y 207/11001; C07K 2319/33; C07K 2319/50; C07K 2319/735; C12N 2830/50; C12N 2840/10

(57) ABSTRACT

In certain aspects, the disclosure relates to a nucleic acid molecule encoding one or more different thanotransmission polypeptides. Vectors (e.g., engineered viruses, plasmids and transposons), cells and pharmaceutical compositions comprising one or more nucleic acid molecules encoding one or more thanotransmission polypeptides are also disclosed. Methods of promoting thanotransmission by a target cell, methods of promoting an immune response in a subject, and methods of treating cancer in a subject are further disclosed.

### RELATED APPLICATIONS

This application claims priority to U.S. Provisional Patent Application No. 63/413,566 filed on Oct. 5, 2022, U.S. Provisional Patent Application No. 63/437,982 filed on Jan. 9, 2023, and U.S. Provisional Patent Application No. US 63/463,850 filed on May 3, 2023, the contents of each of which are incorporated herein in their entirety.

### SUBMISSION OF SEQUENCE LISTING

The Sequence Listing associated with this application is filed in electronic format via EFS-Web and hereby incorporated by reference into the specification in its entirety. The name of the text file containing the Sequence Listing is 129983_01704_Sequence_Listing. The size of the text file is 72,667 bytes, and the text file was created on Oct. 5, 2024.

### BACKGROUND

In metazoans, programmed cell death is an essential genetically programmed process that maintains tissue homeostasis and eliminates potentially harmful cells.

### SUMMARY OF THE INVENTION

In certain aspects, the disclosure relates to a recombinant nucleic acid molecule comprising:
a) a first polynucleotide encoding TRIF or a variant thereof; and
b) a second polynucleotide encoding an additional polypeptide selected from the group consisting of RIPK3, Gasdermin E, vICA, Npro, A238L, vMLKL, and variants thereof, and a dominant negative variant of IKBa.

In some embodiments, the additional polypeptide is selected from the group consisting of RIPK3, Gasdermin E, vICA, Npro, A238L, vMLKL and variants thereof. In some embodiments, the second polynucleotide encodes RIPK3 or a variant thereof. In some embodiments, the second polynucleotide encodes Gasdermin E or a variant thereof. In some embodiments, the second polynucleotide encodes Npro or a variant thereof. In some embodiments, the second polynucleotide encodes A238L or a variant thereof. In some embodiments, the second polynucleotide encodes vMLKL or a variant thereof. In some embodiments, the second polynucleotide encodes a dominant negative variant of IKBa. In some embodiments, the dominant negative variant of IKBa comprises an S32A substitution and an S36A substitution relative to the human wildtype IKBa amino acid sequence of SEQ ID NO: 50.

In some embodiments, the recombinant nucleic acid molecule further comprises a third polynucleotide encoding a polypeptide selected from the group consisting of RIPK3, Gasdermin E, vICA, Npro, A238L, vMLKL, IKBa, and variants thereof, and a dominant negative variant of IKBa, wherein the second polynucleotide and third polynucleotide encode different polypeptides. In some embodiments, the second polynucleotide encodes RIPK3 or a variant thereof, and the third polynucleotide encodes vICA or a variant thereof. In some embodiments, the recombinant nucleic acid molecule is transcribed as a single transcript that encodes the TRIF or variant thereof and the additional polypeptide.

In certain aspects, the disclosure relates to a recombinant nucleic acid molecule comprising:
a) a first polynucleotide encoding IL-12 or a variant thereof; and
b) a second polynucleotide encoding an additional polypeptide selected from the group consisting of TRIF, RIPK3, Gasdermin E, vICA, Npro, A238L, vMLKL, and variants thereof, and a dominant negative variant of IKBa.

In some embodiments, the second polynucleotide encodes TRIF or a variant thereof. In some embodiments, the second polynucleotide encodes Gasdermin E or a variant thereof. In some embodiments, the recombinant nucleic acid molecule further comprises a third polynucleotide encoding a polypeptide selected from the group consisting of TRIF, RIPK3, Gasdermin E, vICA, Npro, A238L, vMLKL, IKBa, and variants thereof, and a dominant negative variant of IKBa, wherein the second polynucleotide and third polynucleotide encode different polypeptides. In some embodiments, the second polynucleotide encodes TRIF or a variant thereof, and the third polynucleotide encodes Gasdermin E or a variant thereof. In some embodiments, the polynucleotide encoding IL-12 or a variant thereof comprises a polynucleotide encoding the p40 subunit of IL-12, a polynucleotide encoding the p35 subunit of IL-12, and a polynucleotide encoding a linker located between the polynucleotide encoding the p40 subunit of IL-12 and the polynucleotide encoding the p35 subunit of IL-12. In some embodiments, the linker is between 5 and 50 amino acid residues in length. In some embodiments, the linker comprises serine and glycine residues. In some embodiments, the linker comprises the amino acid sequence of SEQ ID NO: 59. In some embodiments, the recombinant nucleic acid molecule is transcribed as a single transcript that encodes the IL-12 or variant thereof and the additional polypeptide. In some embodiments, the recombinant nucleic acid molecule is transcribed as two or more separate transcripts, wherein each transcript encodes only one polypeptide. In some embodiments, the recombinant nucleic acid molecule further comprises one or more polynucleotides encoding a 2A peptide. In some embodiments, the recombinant nucleic acid molecule further comprises a polynucleotide encoding a 2A peptide located between the first polynucleotide and the second polynucleotide. In some embodiments, the recombinant nucleic acid molecule further comprises a polynucleotide encoding a 2A peptide located between the second polynucleotide and the third polynucleotide. In some embodiments, the 2A peptide is a P2A peptide.

In certain aspects, the disclosure relates to a recombinant nucleic acid molecule comprising a polynucleotide encoding a polypeptide selected from the group consisting of TRIF, RIPK3, Gasdermin E, vICA, Npro, A238L, vMLKL, and variants thereof, and a dominant negative variant of IKBa. In some embodiments, the dominant negative variant of IKBa comprises an S32A substitution and an S36A substitution relative to the human wildtype IKBa amino acid sequence of SEQ ID NO: 50.

In certain aspects, the disclosure relates to a recombinant nucleic acid molecule comprising a polynucleotide encoding a TRIF variant. In some embodiments, the TRIF variant comprises an N-terminal deletion. In some embodiments, the TRIF variant comprises a deletion of 180 amino acid residues at the N-terminus. In some embodiments, the TRIF variant consists of the amino acid sequence of SEQ ID NO: 14.

In certain aspects, the disclosure relates to a recombinant nucleic acid molecule comprising a polynucleotide encoding a caspase 1 variant. In some embodiments, the caspase 1 variant comprises a self-dimerization domain. In some embodiments, the self-dimerization domain is a C-terminal self-dimerization domain. In some embodiments, the caspase 1 variant comprises the amino acid sequence of SEQ ID NO: 54. In some embodiments, the nucleic acid molecule is a DNA molecule. In some embodiments, the nucleic acid molecule is an RNA molecule. In some embodiments, the RNA molecule is an mRNA molecule. In some embodiments, the RNA molecule is a circular RNA. In some embodiments, the RNA molecule comprises at least one modified uridine. In some embodiments, at least 50% of uridines in the RNA molecule are modified uridines. In some embodiments, each uridine in the RNA molecule is a modified uridine. In some embodiments, the modified uridine is N1-methylpseudouridine. In some embodiments, at least one of the first polynucleotide and second polynucleotide is operably linked to a 3′ untranslated region (3′ UTR), or a polynucleotide encoding a 3′ UTR. In some embodiments, the third polynucleotide is operably linked to a 3′ untranslated region (3′ UTR), or a polynucleotide encoding a 3′ UTR. In some embodiments, each of the first, second and third polynucleotides is operably linked to a 3′ untranslated region (3′ UTR), or a polynucleotide encoding a 3′ UTR. In some embodiments, the 3′ UTR comprises SEQ ID NO: 34 or SEQ ID NO: 35. In some embodiments, the 3′ UTR is operably linked to a polyA tail. In some embodiments, the recombinant nucleic acid molecule further comprises one or more microRNA (miRNA) binding sites, or one or more polynucleotides encoding one or more miRNA binding sites. In some embodiments, the recombinant nucleic acid molecule further comprises at least two different miRNA binding sites, or one or more polynucleotides encoding at least two different miRNA binding sites. In some embodiments, the one or more microRNA (miRNA) binding sites, or the one or more polynucleotides encoding one or more miRNA binding sites, is operably linked to the first, second or third polynucleotide. In some embodiments, the one or more miRNA binding sites, or the one or more polynucleotides encoding the one or more miRNA binding sites, is comprised within the 3′ UTR. In some embodiments, the one or more miRNA binding sites comprise a polynucleotide selected from SEQ ID NO: 36 and SEQ ID NO: 37. In some embodiments, the miRNA binding site comprises SEQ ID NO: 36. In some embodiments, the miRNA binding site comprises SEQ ID NO: 37. In some embodiments, at least one of the first polynucleotide and second polynucleotide is operably linked to a 5′ untranslated region (5′ UTR), or a polynucleotide encoding a 5′ UTR. In some embodiments, the third polynucleotide is operably linked to a 5′ untranslated region (5′ UTR), or a polynucleotide encoding a 5′ UTR. In some embodiments, each of the first, second and third polynucleotides is operably linked to a 5′ untranslated region (5′ UTR), or a polynucleotide encoding a 5′ UTR. In some embodiments, the 5′ UTR comprises SEQ ID NO: 33. In some embodiments, the mRNA molecule further comprises a cap structure operably linked to the 5′ end of the mRNA. In some embodiments, the TRIF or variant thereof and the additional polypeptide are comprised in a fusion protein. In some embodiments, the fusion protein further comprises one or more linkers. In some embodiments, the recombinant nucleic acid molecule further comprises at least one polynucleotide encoding a dimerization domain. In some embodiments, the fusion protein further comprises a dimerization domain. In some embodiments, the dimerization domain is heterologous to the TRIF or variant thereof and the additional polypeptide.

In certain aspects, the disclosure relates to an RNA molecule comprising:
a) a first polynucleotide encoding TRIF or a variant thereof;
b) a second polynucleotide encoding an additional polypeptide selected from the group consisting of RIPK3, Gasdermin E, vICA, Npro, A238L, vMLKL, and variants thereof, and a dominant negative variant of IKBa;
c) one or more 3′ untranslated regions (3′ UTRs), wherein the one or more 3′ UTRs is operably linked to the first polynucleotide or the second polynucleotide; and
d) one or more microRNA (miRNA) binding sites comprised within the one or more 3′ UTRs.

In certain aspects, the disclosure relates to an RNA molecule comprising:
a) a first polynucleotide encoding TRIF or a variant thereof;
b) a second polynucleotide encoding an additional polypeptide selected from the group consisting of RIPK3, Gasdermin E, vICA, Npro, A238L, vMLKL, and variants thereof, and a dominant negative variant of IKBa; and
c) one or more microRNA (miRNA) binding sites operably linked to the first polynucleotide or the second polynucleotide, wherein the one or more miRNA binding sites comprise a polynucleotide selected from SEQ ID NO: 36 and SEQ ID NO: 37.

In certain aspects, the disclosure relates to a recombinant RNA molecule comprising:
a) a first polynucleotide encoding TRIF or a variant thereof;
b) a second polynucleotide encoding an additional polypeptide selected from the group consisting of RIPK3, Gasdermin E, vICA, Npro, A238L, vMLKL, and variants thereof, and a dominant negative variant of IKBa; and
c) one or more 5′ untranslated regions (5′ UTRs) operably linked to the first polynucleotide or the second polynucleotide, wherein the 5′ UTR comprises SEQ ID NO: 33.

In certain aspects, the disclosure relates to an RNA molecule comprising:
a) a 5′ cap structure;
b) a 5′ UTR;
c) a first polynucleotide encoding TRIF or a variant thereof;
d) a second polynucleotide encoding an additional polypeptide selected from the group consisting of RIPK3, Gasdermin E, vICA, Npro, A238L, vMLKL, and variants thereof, and a dominant negative variant of IKBa;
e) a 3′ untranslated region (3′ UTR); and
f) one or more microRNA (miRNA) binding sites comprised within the 3′UTR, wherein the one or more miRNA binding regions comprise a polynucleotide selected from SEQ ID NO: 36 and SEQ ID NO: 37.

In certain aspects, the disclosure relates to an RNA molecule comprising:
a) a first polynucleotide encoding IL-12 or a variant thereof;
b) a second polynucleotide encoding an additional polypeptide selected from the group consisting of TRIF, RIPK3, Gasdermin E, vICA, Npro, A238L, vMLKL, and variants thereof, and a dominant negative variant of IKBa;
c) one or more 3′ untranslated regions (3′ UTRs), wherein the one or more 3′ UTRs is operably linked to the first polynucleotide or the second polynucleotide; and
d) one or more microRNA (miRNA) binding sites comprised within the one or more 3′UTRs.

In certain aspects, the disclosure relates to an RNA molecule comprising:
a) a first polynucleotide encoding IL-12 or a variant thereof;
b) a second polynucleotide encoding an additional polypeptide selected from the group consisting of TRIF, RIPK3, Gasdermin E, vICA, Npro, A238L, vMLKL, and variants thereof, and a dominant negative variant of IKBa; and
c) one or more microRNA (miRNA) binding sites operably linked to the first polynucleotide or the second polynucleotide, wherein the one or more miRNA binding sites comprise a polynucleotide selected from SEQ ID NO: 36 and SEQ ID NO: 37.

In certain aspects, the disclosure relates to a recombinant RNA molecule comprising:
a) a first polynucleotide encoding IL-12 or a variant thereof;
b) a second polynucleotide encoding an additional polypeptide selected from the group consisting of TRIF, RIPK3, Gasdermin E, vICA, Npro, A238L, vMLKL, and variants thereof, and a dominant negative variant of IKBa; and
c) one or more 5′ untranslated regions (5′ UTRs) operably linked to the first polynucleotide or the second polynucleotide, wherein the 5′ UTR comprises SEQ ID NO: 33.

In certain aspects, the disclosure relates to an RNA molecule comprising:
a) a 5′ cap structure;
b) a 5′ UTR;
c) a first polynucleotide encoding IL-12 or a variant thereof;
d) a second polynucleotide encoding an additional polypeptide selected from the group consisting of TRIF, RIPK3, Gasdermin E, vICA, Npro, A238L, vMLKL, and variants thereof, and a dominant negative variant of IKBa;
e) a 3′ untranslated region (3′ UTR); and
f) one or more microRNA (miRNA) binding sites comprised within the 3′UTR, wherein the one or more miRNA binding regions comprise a polynucleotide selected from SEQ ID NO: 36 and SEQ ID NO: 37.

In some embodiments, the RNA molecule further comprises a third polynucleotide encoding a polypeptide selected from the group consisting of TRIF, RIPK3, Gasdermin E, vICA, Npro, A238L, vMLKL, IKBa, and variants thereof, and a dominant negative variant of IKBa, wherein the second polynucleotide and third polynucleotide encode different polypeptides. In some embodiments, the first polynucleotide encodes IL-12 or a variant thereof, the second polynucleotide encodes TRIF or a variant thereof, and the third polynucleotide encodes Gasdermin E or a variant thereof.

In certain aspects, the disclosure relates to a DNA molecule encoding one or more of the preceding RNA molecules.

In certain aspects, the disclosure relates to a lipid nanoparticle (LNP) comprising one or more of the preceding recombinant nucleic acid molecules.

In certain aspects, the disclosure relates to a liposome comprising one or more of the preceding recombinant nucleic acid molecules.

In certain aspects, the disclosure relates to a vector comprising one or more of the preceding recombinant nucleic acid molecules. In some embodiments, the vector is a virus, a plasmid, or a transposon.

In some embodiments, the engineered virus is selected from the group consisting of a Vaccinia virus, a herpes simplex virus (HSV), a vesicular stomatitis virus (VSV) and a respiratory syncytial virus (RSV). In some embodiments, the virus is a wildtype virus. In some embodiments, the wildtype virus is the Indiana strain of VSV. In some embodiments, the virus is a recombinant virus. In some embodiments, the virus is a pseudotyped virus. In some embodiments, the VSV is a pseudotyped virus with a modified G protein. In some embodiments, the VSV is the Indiana strain of VSV comprising a Junin virus G protein

In certain aspects, the disclosure relates to a polypeptide encoded by any one of the preceding recombinant nucleic acid molecules.

In certain aspects, the disclosure relates to a cell comprising one or more of the preceding nucleic acid molecules, vectors and/or polypeptides.

In certain aspects, the disclosure relates to a cell comprising two or more exogenous polynucleotides each encoding a different polypeptide, wherein at least one of the exogenous polynucleotides encodes TRIF or a variant thereof, and at least one of the exogenous polynucleotides encodes a polypeptide selected from the group consisting of: RIPK3, Gasdermin E, vICA, Npro, A238L, vMLKL, variants thereof, and a dominant negative variant of IKBa.

In certain aspects, the disclosure relates to a cell comprising two or more exogenous polynucleotides each encoding a different polypeptide, wherein one or more of the exogenous polynucleotides encodes IL-12 or a variant thereof, and at least one of the exogenous polynucleotides encodes a polypeptide selected from the group consisting of: TRIF, RIPK3, Gasdermin E, vICA, Npro, A238L, vMLKL, variants thereof, and a dominant negative variant of IKBa. In some embodiments, the two or more exogenous polynucleotides are comprised within the same nucleic acid molecule.

In some embodiments, each of the two or more exogenous polynucleotides is comprised in a separate nucleic acid molecule. In some embodiments, the two or more exogenous polynucleotides are DNA molecules. In some embodiments, the DNA molecules are plasmids or transposons. In some embodiments, the two or more exogenous polynucleotides are RNA molecules. In some embodiments, the RNA molecules are mRNAs. In some embodiments, the RNA molecules are circular RNAs. In some embodiments, the RNA molecules comprises at least one modified uridine. In some embodiments, at least 50% of uridines in the RNA molecules are modified uridines. In some embodiments, each uridine in the RNA molecules is a modified uridine. In some embodiments, the modified uridine is N1-methylpseudouridine.

In some embodiments, at least one of the exogenous polynucleotides encodes RIPK3 or a variant thereof. In some embodiments, at least one of the exogenous polynucleotides encodes Gasdermin E or a variant thereof. In some embodiments, at least one of the exogenous polynucleotides encodes Npro or a variant thereof. In some embodiments, at least one of the exogenous polynucleotides encodes A238L or a variant thereof. In some embodiments, at least one of the exogenous polynucleotides encodes vMLKL or a variant thereof. In some embodiments, at least one of the exogenous polynucleotides encodes a dominant negative variant of IKBa. In some embodiments, at least one of the exogenous polynucleotides encodes RIPK3 or a variant thereof, and at least one of the exogenous polynucleotides encodes vICA or a variant thereof. In some embodiments, at least one of the exogenous polynucleotides encodes IL-12 or a variant thereof and at least one of the exogenous polynucleotides encodes TRIF or a variant thereof. In some embodiments, at least one of the exogenous polynucleotides encodes IL-12 or a variant thereof and at least one of the exogenous polynucleotides encodes Gasdermin E or a variant thereof. In some embodiments, at least one of the exogenous polynucleotides encodes IL-12 or a variant thereof, at least one of the exogenous polynucleotides encodes TRIF or a variant thereof, and at least one of the exogenous polynucleotides encodes Gasdermin E or a variant thereof. In some embodiments, the exogenous polynucleotide encoding IL-12 or a variant thereof comprises a polynucleotide encoding the p40 subunit of IL-12, a polynucleotide encoding the p35 subunit of IL-12, and a polynucleotide encoding a linker located between the polynucleotide encoding the p40 subunit of IL-12 and the polynucleotide encoding the p35 subunit of IL-12. In some embodiments, the linker is between 5 and 50 amino acid residues in length. In some embodiments, the linker comprises serine and glycine residues. In some embodiments, the linker comprises the amino acid sequence of SEQ ID NO: 59. In some embodiments, the IL-12 or variant thereof is encoded by two separate polynucleotides, wherein one of the two separate polynucleotides encodes the p35 subunit of IL-12, and one of the two separate polynucleotides encodes the p40 subunit of IL-12.

In some embodiments, at least one of the exogenous polynucleotides is operably linked to a 3′ untranslated region (3′ UTR), or a polynucleotide encoding a 3′ UTR. In some embodiments, each of the exogenous polynucleotides is operably linked to a 3′ untranslated region (3′ UTR), or a polynucleotide encoding a 3′ UTR. In some embodiments, the 3′ UTR is operably linked to a polyA tail. In some embodiments, at least one of the exogenous polynucleotides comprises one or more microRNA (miRNA) binding sites, or one or more polynucleotides encoding one or more miRNA binding sites. In some embodiments, at least one of the exogenous polynucleotides further comprises at least two different miRNA binding sites, or one or more polynucleotides encoding at least two different miRNA binding sites. In some embodiments, the one or more miRNA binding sites, or the one or more polynucleotides encoding the one or more miRNA binding sites, are comprised within the 3′ UTR. In some embodiments, the one or more miRNA binding sites comprise a polynucleotide selected from SEQ ID NO: 36 and SEQ ID NO: 37. In some embodiments, the miRNA binding site comprises SEQ ID NO: 36. In some embodiments, the miRNA binding site comprises SEQ ID NO: 37. In some embodiments, at least one of the exogenous polynucleotides is operably linked to a 5′ untranslated region (5′ UTR), or a polynucleotide encoding a 5′ UTR. In some embodiments, each of the exogenous polynucleotides is operably linked to a 5′ untranslated region (5′ UTR), or a polynucleotide encoding a 5′ UTR.

In certain aspects, the disclosure relates to a pharmaceutical composition comprising a) any one of the preceding recombinant nucleic acid molecules, liposomes, vectors, or cells, and b) a pharmaceutically acceptable carrier.

In certain aspects, the disclosure relates to a pharmaceutical composition comprising:
(a) two or more recombinant polynucleotides each encoding a different polypeptide, wherein at least one of the recombinant polynucleotides encodes TRIF or a variant thereof, and at least one of the recombinant polynucleotides encodes a polypeptide selected from the group consisting of: RIPK3, Gasdermin E, vICA, Npro, A238L, vMLKL, and variants thereof, and a dominant negative variant of IKBa; and
(b) a pharmaceutically acceptable carrier.

In certain aspects, the disclosure relates to a pharmaceutical composition comprising:
(a) two or more recombinant polynucleotides each encoding a different polypeptide, wherein at least one of the recombinant polynucleotides encodes IL-12 or a variant thereof, and at least one of the recombinant polynucleotides encodes a polypeptide selected from the group consisting of: TRIF, RIPK3, Gasdermin E, vICA, Npro, A238L, vMLKL, and variants thereof, and a dominant negative variant of IKBa; and
(b) a pharmaceutically acceptable carrier.

In some embodiments, the two or more recombinant polynucleotides in the pharmaceutical composition are comprised within the same nucleic acid molecule. In some embodiments, each of the two or more recombinant polynucleotides in the pharmaceutical composition is comprised in a separate nucleic acid molecule. In some embodiments, the two or more recombinant polynucleotides are DNA molecules. In some embodiments, the DNA molecules are plasmids or transposons. In some embodiments, the two or more recombinant polynucleotides are comprised within a virus. In some embodiments, the virus is a wildtype virus. In some embodiments, the wildtype virus is the Indiana strain of VSV. In some embodiments, the virus is a recombinant virus. In some embodiments, the virus is a pseudotyped virus. In some embodiments, the VSV is a pseudotyped virus with a modified G protein. In some embodiments, the VSV is the Indiana strain of VSV comprising a Junin virus G protein. In some embodiments, the two or more recombinant polynucleotides are RNA molecules. In some embodiments, the RNA molecules are mRNAs. In some embodiments, the RNA molecules are circular RNAs. In some embodiments, the RNA molecules comprises at least one modified uridine. In some embodiments, at least 50% of uridines in the RNA molecules are modified uridines. In some embodiments, each uridine in the RNA molecules is a modified uridine. In some embodiments, the modified uridine is N1-methylpseudouridine. In some embodiments, at least one of the recombinant polynucleotides encodes RIPK3 or a variant thereof. In some embodiments, at least one of the recombinant polynucleotides encodes Gasdermin E or a variant thereof. In some embodiments, at least one of the recombinant polynucleotides encodes Npro or a variant thereof. In some embodiments, at least one of the recombinant polynucleotides encodes A238L or a variant thereof. In some embodiments, at least one of the recombinant polynucleotides encodes vMLKL or a variant thereof. In some embodiments, at least one of the recombinant polynucleotides encodes a dominant negative variant of IKBa. In some embodiments, at least one of the recombinant polynucleotides encodes RIPK3 or a variant thereof, and at least one of the recombinant polynucleotides encodes vICA or a variant thereof. In some embodiments, at least one of the recombinant polynucleotides encodes IL-12 or a variant thereof and at least one of the recombinant polynucleotides encodes TRIF or a variant thereof. In some embodiments, at least one of the recombinant polynucleotides encodes IL-12 or a variant thereof and at least one of the recombinant polynucleotides encodes Gasdermin E or a variant thereof. In some embodiments, at least one of the recombinant polynucleotides encodes IL-12 or a variant thereof, at least one of the recombinant polynucleotides encodes TRIF or a variant thereof, and at least one of the recombinant polynucleotides encodes Gasdermin E or a variant thereof. In some embodiments, the recombinant polynucleotide encoding IL-12 or a variant thereof comprises a polynucleotide encoding the p40 subunit of IL-12, a polynucleotide encoding the p35 subunit of IL-12, and a polynucleotide encoding a linker located between the polynucleotide encoding the p40 subunit of IL-12 and the polynucleotide encoding the p35 subunit of IL-12. In some embodiments, the linker is between 5 and 50 amino acid residues in length. In some embodiments, the linker comprises serine and glycine residues. In some embodiments, the linker comprises the amino acid sequence of SEQ ID NO: 59. In some embodiments, the IL-12 or variant thereof is encoded by two separate polynucleotides, wherein one of the two separate polynucleotides encodes the p35 subunit of IL-12, and one of the two separate polynucleotides encodes the p40 subunit of IL-12. In some embodiments, at least one of the recombinant polynucleotides is operably linked to a 3′ untranslated region (3′ UTR), or a polynucleotide encoding a 3′ UTR. In some embodiments, the 3′ UTR is operably linked to a polyA tail.

In some embodiments, at least one of the recombinant polynucleotides comprises one or more microRNA (miRNA) binding sites, or one or more polynucleotides encoding one or more miRNA binding sites. In some embodiments, at least one of the recombinant polynucleotides further comprises at least two different miRNA binding sites, or one or more polynucleotides encoding at least two different miRNA binding sites. In some embodiments, the one or more miRNA binding sites, or the one or more polynucleotides encoding the one or more miRNA binding sites, are comprised within the 3′ UTR. In some embodiments, the one or more miRNA binding sites comprise a polynucleotide selected from SEQ ID NO: 36 and SEQ ID NO: 37. In some embodiments, the miRNA binding site comprises SEQ ID NO: 36. In some embodiments, the miRNA binding site comprises SEQ ID NO: 37. In some embodiments, at least one of the recombinant polynucleotides is operably linked to a 5′ untranslated region (5′ UTR). In some embodiments, each of the recombinant polynucleotides is operably linked to a 5′ untranslated region (5′ UTR).

In certain aspects, the disclosure relates to a method of delivering one or more nucleic acid molecules to a subject, the method comprising administering any one of the preceding pharmaceutical compositions to the subject.

In certain aspects, the disclosure relates to a method of promoting thanotransmission in a subject, the method comprising administering any one of the preceding pharmaceutical compositions to the subject in an amount and for a time sufficient to promote thanotransmission.

In certain aspects, the disclosure relates to a method of increasing immune response in a subject in need thereof, the method comprising administering any one of the preceding pharmaceutical compositions to the subject in an amount and for a time sufficient to increase immune response in the subject. In some embodiments, administration of the pharmaceutical composition to the subject increases immune response relative to a subject that is administered a pharmaceutical composition that comprises a polynucleotide encoding TRIF or a variant thereof, but does not comprise a polynucleotides encoding the additional polypeptide. In some embodiments, administration of the pharmaceutical composition to the subject increases immune response relative to a subject that is administered a pharmaceutical composition that comprises a polynucleotide encoding IL-12 or a variant thereof, but does not comprise a polynucleotides encoding the additional polypeptide. In some embodiments, the increasing immune response comprises increasing the expression and/or activity of one or more proteins selected from the group consisting of NFκB, IRF, NFAT, myd88, AP-1, STAT1, STAT2, STAT3, STAT 4, STAT 5, IRAK1, IRAK2, IRAK 3 and IRAK 4. In some embodiments, the increasing immune response comprises increasing one or more of NFkB activity and IRF activity. In some embodiments, the increasing immune response comprises increasing cytokine or chemokine production and/or activity. In some embodiments, the increasing immune response comprises increasing immune cell mediated cytotoxicity. In some embodiments, the increasing immune response comprises increasing expression of a receptor-ligand pairing. In some embodiments, the receptor-ligand pairing is selected from the group consisting of co-stimulatory molecules CD80 and CD86, FAS:FASL, and CD40L:CD40. In some embodiments, the increasing immune response comprises reducing anti-inflammatory signals and/or anti-inflammatory cells. In some embodiments, the anti-inflammatory signals are selected from IL-10 and TGF beta. In some embodiments, the anti-inflammatory cells are selected from T regulatory cells and myeloid derived suppressor cells. In some embodiments, the increased immune response comprises increased HLA/MHC antigen presentation or antigen release by target cells. In some embodiments, the target cells are tumor cells. In some embodiments, the increased immune response comprises reduced expression of anti-immune factors. In some embodiments, the anti-immune factors are selected from the group consisting of immune checkpoint molecules, suppressive cytokines, and suppressive transcription factors. In some embodiments, the immune checkpoint molecule is PDL1. In some embodiments, the suppressive transcription factor is selected from SOCS1 and SOCS3. In some embodiments, the increased immune response comprises any one or more of: activation of NK cells, activation of antigen-presenting dendritic cells, activation of CD4+ T cells, activation of CD8+ T cells, and conversion of immunosuppressive macrophages to immune-stimulatory macrophages.

In certain aspects, the disclosure relates to a method of treating a cancer in a subject in need thereof, the method comprising administering any one of the preceding pharmaceutical compositions to the subject in an amount and for a time sufficient to treat the cancer.

In some embodiments, the pharmaceutical composition is administered intravenously to the subject. In some embodiments, the pharmaceutical composition is delivered to the subject through lipofection. In some embodiments, the lipofection is RNA lipofection. In some embodiments, the lipofection is DNA lipofection.

In some embodiments, administering the pharmaceutical composition to the subject reduces tumor growth in the subject relative to a subject that is not administered the pharmaceutical composition. In some embodiments, administering the pharmaceutical composition to the subject reduces tumor growth in the subject relative to a subject that is administered a pharmaceutical composition that comprises a polynucleotide encoding TRIF or a variant thereof, but does not comprise a polynucleotides encoding the additional polypeptide. In some embodiments, administering the pharmaceutical composition to the subject reduces tumor growth in the subject relative to a subject that is administered a pharmaceutical composition that comprises a polynucleotide encoding IL-12 or a variant thereof, but does not comprise a polynucleotides encoding the additional polypeptide. In some embodiments, administering the pharmaceutical composition to the subject reduces proliferation of cancer cells in the subject. In some embodiments, the proliferation of the cancer cells is a hyperproliferation of the cancer cells resulting from a cancer therapy administered to the subject. In some embodiments, administering the pharmaceutical composition to the subject reduces metastasis of cancer cells in the subject. In some embodiments, administering the pharmaceutical composition to the subject reduces neovascularization of a tumor in the subject. In some embodiments, administering the pharmaceutical composition to the subject upregulates tumor cell MHC-1 expression in the subject. In some embodiments, treating a cancer comprises any one or more of reduction in tumor burden, reduction in tumor size, inhibition of tumor growth, achievement of stable cancer in a subject with a progressive cancer prior to treatment, increased time to progression of the cancer, and increased time of survival. In some embodiments, the cancer exhibits reduced RIPK3 expression. In some embodiments, the cancer is a solid tumor. In some embodiments, the tumor is an immunologically cold tumor. In some embodiments, the cancer is not responsive to a PD-1 inhibitor. In some embodiments, the PD-1 inhibitor is an anti-PD-1 antibody. In some embodiments, the cancer is selected from the group consisting of melanoma, colorectal cancer, lung cancer, head and neck cancer, gastric cancer, ovarian cancer, prostate cancer, adrenocortical cancer and breast cancer. In some embodiments, the cancer is colon cancer. In some embodiments, the cancer is melanoma. In some embodiments, the method further comprises administering an anti-neoplastic agent to the subject.

In some embodiments, administration of the pharmaceutical composition to the subject increases survival time and/or reduces tumor growth relative to a subject that is administered a pharmaceutical composition that comprises a polynucleotide encoding TRIF or a variant thereof, but does not comprise a polynucleotide encoding the additional polypeptide. In some embodiments, administration of the pharmaceutical composition to the subject increases survival time and/or reduces tumor growth relative to a subject that is administered a pharmaceutical composition that comprises a polynucleotide encoding IL-12 or a variant thereof, but does not comprise a polynucleotide encoding the additional polypeptide.

### BRIEF DESCRIPTION OF THE FIGURES

FIGS. 1A and 1B show relative viability of CT-26 mouse colon carcinoma cells following induction of thanotransmission.

FIGS. 2A and 2B show the effects of cell turnover factors (CTFs) generated from CT-26 mouse colon carcinoma cells following induction of thanotransmission polypeptide expression (e.g., TRIF expression alone or in combination with RIPK3 (cR3) and/or Gasdermin E (cGE)) on stimulation of IFN-related gene activation in macrophages. In FIG. 2A, the Tet-inducible RIPK3 is designated as “RIPK3”, and the RIPK3 construct containing a constitutive PGK promoter is designated as “PGK_RIPK3”. In FIG. 2B, for each thanotransmission module, the treatment groups from left to right are control (CTL), doxycycline (Dox), and doxycycline+B/B homodimerizer (Dox+Dimerizer).

FIG. 3 shows the effects of cell turnover factors (CTFs) generated from CT-26 mouse colon carcinoma cells following induction of TRIF, RIPK3 or TRIF and RIPK3 expression on stimulation of expression of activation markers in bone marrow derived dendritic cells (BMDCs). MFI is mean-fluorescent intensity.

FIGS. 4A, 4B and 4C show the effects of thanotransmission polypeptide expression on survival of mice implanted with CT-26 mouse colon carcinoma cells. “CT26-TF” represents CT-26 cells expressing TRIF alone, and “CT26-P_R3” represents cells expressing RIPK3 alone. In FIG. 4B, all mice were treated with an anti-PD1 antibody.

FIG. 5A shows relative NF-kB activity in THP-1 Dual cells treated with cell culture from U937 leukemia cells expressing various thanotransmission payloads and treated with caspase inhibitor (Q-VD-Oph) alone or in combination with RIPK3 inhibitor (GSK872). FIGS. 5B and 5C show relative IRF activity in THP-1 Dual cells treated with cell culture from U937 leukemia cells expressing various thanotransmission payloads and treated with caspase inhibitor (Q-VD-Oph) alone or in combination with RIPK3 inhibitor (GSK872). The U937 cells were also treated with doxycycline to induce thanotransmission polypeptide expression, alone or in combination with B/B homodimerizer to induce dimerization. In FIGS. 5A-5C, + indicates U937 cells treated with doxycycline, and ++ indicates U937 cells treated with doxycycline and B/B homodimerizer.

FIG. 6A shows relative viability of CT-26 mouse colon carcinoma cells expressing thanotransmission polypeptides alone or in combination with caspase inhibitors. FIG. 6B shows the effects of cell turnover factors (CTFs) generated from CT-26 mouse colon carcinoma cells following induction of thanotransmission polypeptide expression alone or in combination with caspase inhibitors on stimulation of IFN-related gene activation in macrophages. FIG. 6C shows the effect of TRIF+RIPK3 expression alone or in combination with caspase inhibitors on survival of mice implanted with CT-26 mouse colon carcinoma cells.

FIG. 7 shows cell viability in HT29 cells after expression of TRIF variants and controls.

FIG. 8A shows IRF activity in THP1-Dual cells cultured with supernatant of HT29 cells expressing specific TRIF variants. FIG. 8B shows NFkB activity in THP1-Dual cells cultured with supernatant of HT29 cells expressing specific TRIF variants.

FIG. 9 shows cell viability in A375 cells expressing TRIF variants and controls.

FIG. 10 shows IRF activity (top panel) and NFkB activity (bottom panel) in THP1-Dual cells cultured with the supernatant of A375 cells expressing specific TRIF variants.

FIG. 11 shows the effect of mini TRIF+GSDME expression on tumor growth in mice implanted with CT-26 mouse colon carcinoma cells.

FIG. 12 shows the effect of mini TRIF+GSDME expression on survival of mice implanted with CT-26 mouse colon carcinoma cells.

FIG. 13 shows the effect of mini TRIF+RIPK3 expression on tumor growth in mice implanted with CT-26 mouse colon carcinoma cells.

FIG. 14 shows the effect of mini TRIF+RIPK3 expression on survival of mice implanted with CT-26 mouse colon carcinoma cells.

FIG. 15 shows cell death in mouse breast cancer 4T1 cells (left panel), and IRF activity in J774-Dual™ cells (right panel) treated with culture medium from the cancer cells. The cancer cells were treated with a replication incompetent adenovirus 5 (E1 and E3 region deleted) encoding mRIPK3, TRIF-mRIPK3, or TRIF-mRIPK3-vICA, or a mock adenovirus control.

FIG. 16 shows cell death in mouse colon cancer MC38 cells (left panel), and IRF activity in J774-Dual™ cells (right panel) treated with culture medium from the cancer cells. The cancer cells were treated with a replication incompetent adenovirus 5 (E1 and E3 region deleted) encoding mRIPK3, TRIF-mRIPK3, or TRIF-mRIPK3-vICA, or a mock adenovirus control.

FIG. 17 shows cell death in mouse pancreatic cancer Pan02 cells (left panel), and IRF activity in J774-Dual™ cells (right panel) treated with culture medium from the cancer cells. The cancer cells were treated with a replication incompetent adenovirus 5 (E1 and E3 region deleted) encoding mRIPK3, TRIF-mRIPK3, or TRIF-mRIPK3-vICA, or a mock adenovirus control.

FIG. 18A shows exemplary constructs comprising one, two or three genes encoding thanotransmission polypeptides for mRNA delivery. FIG. 18B shows exemplary constructs comprising genes encoding thanotransmission polypeptides for viral delivery.

FIG. 19 shows cell viability in cancer cells transfected with mRNA encoding TRIF, TRIF+RIPK3, TRIF+RIPK3+vICA, or TRIF+GSDME.

FIGS. 20A and 20B show cytokine and chemokine secretion from cancer cells transfected with mRNA encoding TRIF, TRIF+RIPK3, TRIF+RIPK3+vICA, or TRIF+GSDME. The columns from left to right are Triton X (control), TRIF, TRIF+RIPK3, TRIF+RIPK3+vICA, and TRIF+GSDME.

FIG. 21 shows tumor growth in female C57BL/6 mice implanted with B16F10 murine melanoma cells. Tumors were injected with mRNA encoding TRIF+RIPK3, TRIF+RIPK3+vICA, TRIF+GSDME, or firefly luciferase (Control mRNA), or with an equivalent volume of PBS. TGI=tumor growth inhibition. The X-axis shows days after the first injection, which was administered on Day 0. The arrows indicate the timing of the four intratumoral injections, which were administered on Days 0, 1, 2, and 3.

FIG. 22 shows a sequence alignment of an N-terminal fragment of human wildtype caspase 1 protein (Protein 1; SEQ ID NO: 53) and a caspase 1 variant (CASP1FV) comprising a self-dimerization domain at its C-terminus (Protein 2; SEQ ID NO: 54).

FIG. 23 shows tumor growth in female Balb/c mice implanted with CT26 murine colorectal carcinoma cells expressing TRIF+Npro under control of a doxycycline-inducible promoter.

FIG. 24 shows tumor growth in female Balb/c mice implanted with CT26 murine colorectal carcinoma cells expressing TRIF+GSDME under control of a doxycycline-inducible promoter.

FIG. 25 shows tumor growth in female Balb/c mice implanted with CT26 murine colorectal carcinoma cells expressing a caspase 1 variant (CASP1FV, containing a self-dimerization domain at its C-terminus) under control of a doxycycline-inducible promoter.

FIG. 26 shows tumor growth in female Balb/c mice implanted with CT26 murine colorectal carcinoma cells transfected with an empty Tet3G vector as a negative control.

FIGS. 27A and 27B show tumor growth in female C57BL/6 mice implanted with B16F10 murine melanoma cells. FIG. 27A shows the volume of tumors that were injected with mRNA encoding mIL-12, or firefly luciferase (Control mRNA). Responses were compared with treatment using a monoclonal antibody to PD-1, delivered intraperitoneally twice per week (b.i.w.). FIG. 27B shows the volume of tumors that were injected with mRNA encoding mIL-12, mIL-12+hTRIF+hGSDME, or firefly luciferase (Control mRNA). TGI=tumor growth inhibition; CR=complete regression. The X-axis shows days after the first injection, which was administered on Day 0. The arrows indicate the timing of the four intratumoral injections, which were administered on Days 0, 1, 2, and 3.

FIGS. 28A and 28B show murine colorectal carcinoma (CT26) tumor cell death induced by expression of TRIF and Gasdermin E.

FIG. 29 shows activation of NK cells and antigen presenting dendritic cells induced by TRIF and Gasdermin E.

FIG. 30 shows upregulation of tumor cell MHC-I expression induced by TRIF and Gasdermin E.

FIG. 31 shows conversion of macrophages from immunosuppressive to immune-stimulatory induced by TRIF and Gasdermin E.

FIG. 32 shows activation of CD4+ and CD8 T cells induced by TRIF and Gasdermin E.

FIG. 33 shows tumor growth in female C57BL/6 mice implanted with CT26 murine colorectal carcinoma cells expressing a TRIF variant (TRIF_180; human TRIF with a deletion of amino acid residues 1-180) under control of a doxycycline-inducible promoter. The box labelled “Dox” indicates the days on which doxycycline was administered to the mice.

FIGS. 34A and 34B show that expression of thanotransmission polypeptides TRIF and GSDME drives activation of both antigen specific and non-specific CD8+ T cells within CT26 tumors and are required for efficacy. Day 3 following doxycycline induced thanotransmission polypeptide expression of TRIF and GSDME, the significant activation of tumor infiltrating CD8+ T cells (A) and antigen specific CD8+gp70tet+ T cells (B), is evident by the enhanced surface expression of CD69. In a separate, but identical experimental setup, the depletion of CD8+ T cells through the use of a monoclonal depletion antibody demonstrates that CD8+ T cells are absolutely require for the efficacy of induced thanotransmission polypeptide expression of TRIF and GSDME on tumor control and clearance. Error bars denote SEM. * p<0.05; ** p<0.01; ***p<0.001; **** p<0.0001.

FIGS. 35A and 35B show that expression of thanotransmission polypeptides TRIF and GSDME drives significant abscopal effect in distal tumors of mice implanted with CT26 tumors. Mice were implanted in both the right and left flank and randomized when tumors reach 100 mm³ (right) and 40 mm3 (left). (A) Shows right flank tumor growth in female BALB/c mice implanted with CT26 murine colorectal carcinoma cells expressing thanotransmission polypeptide TRIF and GSDME or Tet3g control. (B) Shows left flank tumor growth in female BALB/c mice implanted with wild type CT26. In both, a subgroup of TRIF+GSDME expressing mice were administered PD1 antibody starting at day 16 and administered every three days for a total of three doses.

FIGS. 36A and 36B show that LNP-mRNA delivery of thanotransmission polypeptides TRIF, GSDME and IL-12 drives a significant abscopal effect in distal tumors of mice implanted with B16F10 tumors. FIG. 36 shows the effects of thanotransmission polypeptide expression on tumor growth in both the primary treated (right flank) (A) and untreated distal (left flank) tumors (B).

FIGS. 37A-37D show that in vitro delivery of thanotransmission polypeptides TRIF and GSDME along with IL-12 drives immunogenic cell death. (A) shows the direct cytotoxicity of TRIF+GSDME+IL-12, as compared with IL-12 alone or an apoptosis inducer after 24 hrs; (B) shows the membrane permeability as measured by Sytox positivity using an Inucycte; (C) shows the release of HMGB1, a typical damage-associated molecular pattern (DAMP) that is passively released during various types of cell death, as measured by ELISA at 24 h post polypeptide delivery by mRNA; (D) shows the differential production of pro-inflammatory cytokines and chemokines as determined using a Biolegend LegendPlex kit and expressed as Log 2 fold change from the average expression of the mRNA control. “Apop” indicates the apoptosis inducer, and “GE” indicates GSDME. Three replicates for each condition were included. Error bars denote SEM. * p<0.05; ** p<0.01; ***p<0.001; **** p<0.0001.

FIGS. 38A-38C show that LNP-mRNA delivery of thanotransmission polypeptides TRIF and GSDME along with IL-12 drives significant early tumor shrinkage and activation of antigen specific CD8+ T cells. (A) shows the differential tumor growth patterns for mice treated with LNP-mRNA IL-12 and LNP-mRNAs for TRIF GSDME, and IL-12; (B) shows the total activation of CD8+ T cells within the tumor, as determined by the expression of CD69; (C) shows the activation of gp100+CD8+ T cells within the tumor, as determined by the expression of CD69. Error bars denote SEM. * p<0.05; ** p<0.01; ***p<0.001; **** p<0.0001.

FIG. 39 shows NK cell recruitment in a CT26 tumor model following three days of Dox exposure. The frequency of NK cells is expressed as the percentage of NKp46+ cells among the total CD45+ cells taken from isolated tumor samples. The negative control (Neg Ctrl) is on the left, and TRIF+GSDME is on the right.

### DETAILED DESCRIPTION

The present disclosure relates to recombinant nucleic acid molecules encoding polypeptides that promote thanotransmission by a target cell. Thanotransmission is a process of communication between cells, e.g., between a target signaling cell and a responding cell, that is a result of activation of a cell turnover pathway in the target cell, which signals the responding cell to undergo a biological response. Thanotransmission may be induced in a target cell by modulation of cell turnover pathway genes through, for example, contacting the target cell with nucleic acid molecules encoding the thanotransmission polypeptides (e.g., referred to as genetic switches herein) described herein. The target cell in which a cell turnover pathway has been activated may signal a responding cell through factors actively released by the target cell, or through intracellular factors of the target cell that become exposed to the responding cell during the turnover (e.g., cell death) of the target cell. In some embodiments, the thanotransmission polypeptides described herein are comprised within a fusion protein. In some embodiments, each of the thanotransmission polypeptides is expressed as a separate polypeptide.

The present disclosure also relates to methods of promoting thanotransmission in a subject, the method comprising administering the thanotransmission polypeptides and/or nucleic acid molecules encoding the thanotransmission polypeptides to the subject in an amount and for a time sufficient to promote thanotransmission. Methods of increasing immune response and methods of treating cancer comprising administering the thanotransmission polypeptides and/or nucleic acid molecules encoding the thanotransmission polypeptides are also described.

### I. Definitions

The terms “administer”, “administering” or “administration” include any method of delivery of a pharmaceutical composition or agent into a subject's system or to a particular region in or on a subject.

As used herein, “administering in combination”, “co-administration” or “combination therapy” is understood as administration of two or more active agents using separate formulations or a single pharmaceutical formulation, or consecutive administration in any order such that, there is a time period while both (or all) active agents overlap in exerting their biological activities. It is contemplated herein that one active agent (e.g., a pharmaceutical composition comprising one or more recombinant nucleic acid molecules encoding thanotransmission polypeptides as described herein) can improve the activity of a second therapeutic agent (e.g., an immunotherapeutic), for example, can sensitize target cells, e.g., cancer cells, to the activities of the second therapeutic agent or can have a synergistic effect with the second therapeutic agent. “Administering in combination” does not require that the agents are administered at the same time, at the same frequency, or by the same route of administration. As used herein, “administering in combination”, “co-administration” or “combination therapy” includes administration of a recombinant nucleic acid molecule encoding one or more thanotransmission polypeptides as described herein with one or more additional therapeutic agents, e.g., an immunotherapeutic (e.g. an immune checkpoint modulator). Examples of immunotherapeutics are provided herein. In some embodiments, genetic switches encoding one or more thanotransmission polypeptides are co-administered sequentially or concurrently with one or more nucleic acid molecules encoding one or more cytokines, e.g., IL-12.

As used herein, the term “anellovector” refers to a vector that comprises sufficient nucleic acid sequence derived from or highly similar to (e.g., at least 85%, 90%, 95%, 96%, 97%, 98%, 99%, or 100% identical to) an Anellovirus genome sequence or a contiguous portion thereof to allow packaging into a proteinaceous exterior (e.g., a capsid), and further comprises a heterologous sequence. In some embodiments, the anellovector is a viral vector or a naked nucleic acid. In some embodiments, the anellovector comprises at least about 50, 60, 70, 71, 72, 73, 74, 75, 80, 90, 100, 150, 200, 300, 400, 500, 600, 700, 800, 900, 1000, 1100, 1200, 1300, 1400, 1500, 1600, 1700, 1800, 1900, 2000, 2500, 3000, or 3500 consecutive nucleotides of a native Anellovirus sequence or a sequence highly similar (e.g., at least 85%, 90%, 95%, 96%, 97%, 98%, 99%, or 100% identical) thereto. In some embodiments, the anellovector further comprises one or more of an Anellovirus ORF1, ORF2, or ORF3. In some embodiments, the heterologous sequence comprises a multiple cloning site, comprises a heterologous promoter, comprises a coding region for a therapeutic protein, or encodes a therapeutic nucleic acid. In some embodiments, the capsid is a wild-type Anellovirus capsid. Anellovectors are described, for example, in U.S. Pat. No. 11,166,996, which is incorporated by reference herein in its entirety.

As used herein, the term “circular RNA” refers to a polyribonucleotide that forms a circular structure through covalent or non-covalent bonds. Circular RNAs are described, for example, in U.S. Pat. No. 11,160,822, which is incorporated by reference herein in its entirety.

As used herein, the terms “increasing” and “decreasing” refer to modulating resulting in, respectively, greater or lesser amounts, function or activity of a parameter relative to a reference. For example, subsequent to administration of a composition described herein, a parameter (e.g., activation of IRF, activation of NF-κB, activation of macrophages, size or growth of a tumor) may be increased or decreased in a subject by at least 5%, 10%, 15%, 20%, 25%, 30%, 35%, 40%, 45%, 50^{%}, 55%, 60%, 65%, 70%, 75%, 80%, 85%, 90%, 95% or 98% or more relative to the amount of the parameter prior to administration. Generally, the metric is measured subsequent to administration at a time that the administration has had the recited effect, e.g., at least one day, one week, one month, 3 months, 6 months, after a treatment regimen has begun. Similarly, pre-clinical parameters (such as activation of NF-xB or IRF of cells in vitro, and/or reduction in tumor burden of a test mammal, by a composition described herein) may be increased or decreased by at least 5%, 10%, 15%, 20%, 25%, 30%, 35%, 40%, 45%, 50%, 55%, 60%, 65%, 70%, 75%, 80%, 85%, 90%, 95% or 98% or more relative to the amount of the parameter prior to administration.

As used herein, “an anti-neoplastic agent” refers to a drug used for the treatment of cancer. Anti-neoplastic agents include chemotherapeutic agents (e.g., alkylating agents, antimetabolites, anti-tumor antibiotics, topoisomerase inhibitors, mitotic inhibitors corticosteroids, and enzymes), biologic anti-cancer agents, and immune checkpoint modulators.

A “cancer treatment regimen” or “anti-neoplastic regimen” is a clinically accepted dosing protocol for the treatment of cancer that includes administration of one or more anti-neoplastic agents to a subject in specific amounts on a specific schedule.

A “fusogenic protein” as used herein refers to any heterologous protein capable of promoting fusion of a cell infected with a virus to another cell. Examples of fusogenic proteins include VSV-G, syncitin-1 (from human endogenous retrovirus-W (HERV-W)) or syncitin-2 (from HERVFRDE1), paramyxovirus SV5-F, measles virus-H, measles virus-F, RSV-F, the glycoprotein from a retrovirus or lentivirus, such as gibbon ape leukemia virus (GALV), murine leukemia virus (MLV), Mason-Pfizer monkey virus (MPMV) and equine infectious anemia virus (EIAV) with the R transmembrane peptide removed (R-versions).

The term “heterologous” as used herein refers to a combination of elements that do not naturally occur in combination. For example, a polynucleotide that is heterologous to a virus or target cell refers to a polynucleotide that does not naturally occur in the virus or target cell, or that occurs in a position in the virus or target cell that is different from the position at which it occurs in nature. A polypeptide that is heterologous to a target cell refers to a polypeptide that does not naturally occur in the target cell, or that is expressed from a polynucleotide that is heterologous to the target cell.

As used herein, an “immune checkpoint” or “immune checkpoint molecule” is a molecule in the immune system that modulates a signal. An immune checkpoint molecule can be a stimulatory checkpoint molecule, i.e., increase a signal, or inhibitory checkpoint molecule, i.e., decrease a signal. A “stimulatory checkpoint molecule” as used herein is a molecule in the immune system that increases a signal or is co-stimulatory. An “inhibitory checkpoint molecule”, as used herein is a molecule in the immune system that decreases a signal or is co-inhibitory.

As used herein, an “immune checkpoint modulator” is an agent capable of altering the activity of an immune checkpoint in a subject. In certain embodiments, an immune checkpoint modulator alters the function of one or more immune checkpoint molecules including, but not limited to, CD27, CD28, CD40, CD122, OX40, GITR, ICOS, 4-1BB, ADORA2A, B7-H3, B7-H4, BTLA, CTLA-4, IDO, KIR, LAG-3, PD-1, PD-L1, PD-L2, TIM-3, and VISTA. The immune checkpoint modulator may be an agonist or an antagonist of the immune checkpoint. In some embodiments, the immune checkpoint modulator is an immune checkpoint binding protein (e.g., an antibody, antibody Fab fragment, divalent antibody, antibody drug conjugate, scFv, fusion protein, bivalent antibody, or tetravalent antibody). In other embodiments, the immune checkpoint modulator is a small molecule. In a particular embodiment, the immune checkpoint modulator is an anti-PD1, anti-PD-L1, or anti-CTLA-4 binding protein, e.g., antibody or antibody fragment.

An “immunotherapeutic” as used herein refers to a pharmaceutically acceptable compound, composition or therapy that induces or enhances an immune response.

Immunotherapeutics include, but are not limited to, immune checkpoint modulators, Toll-like receptor (TLR) agonists, cell-based therapies, cytokines and cancer vaccines. In some embodiments, for example, non-limiting exemplary immunotherapeutic cytokines include IL-2, IL-12, IL-15, IL-18, and IL-21. In some embodiments, the immunotherapeutic cytokine is IL-12.

The term “linker” as used herein refers to a peptide located between two polypeptides comprising a fusion protein, such that the linker is not derived from either polypeptide in the fusion protein. Linkers may be incorporated into fusion proteins as spacers in order to promote proper protein folding and stability of the component protein moieties, to improve protein expression, or to enable better bioactivity of the two fusion partners (Chen, et al., 2013, Adv Drug Deliv Rev. 65(10):1357-69). In some embodiments, the linker is a flexible (unstructured) linker. In some embodiments, the linker is a rigid (structured) linker.

As used herein, “oncological disorder” or “cancer” or “neoplasm” refer to all types of cancer or neoplasm found in humans, including, but not limited to: leukemias, lymphomas, melanomas, carcinomas and sarcomas. As used herein, the terms “oncological disorder”, “cancer,” and “neoplasm,” used interchangeably and in either the singular or plural form, refer to cells that have undergone a malignant transformation that makes them pathological to the host organism. Primary cancer cells (that is, cells obtained from near the site of malignant transformation) can be readily distinguished from non-cancerous cells by well-established techniques, particularly histological examination. The definition of a cancer cell, as used herein, includes not only a primary cancer cell, but also cancer stem cells, as well as cancer progenitor cells or any cell derived from a cancer cell ancestor. This includes metastasized cancer cells, and in vitro cultures and cell lines derived from cancer cells.

Specific criteria for the staging of cancer are dependent on the specific cancer type based on tumor size, histological characteristics, tumor markers, and other criteria known by those of skill in the art. Generally, cancer stages can be described as follows: (i) Stage 0, Carcinoma in situ; (ii) Stage I, Stage II, and Stage III, wherein higher numbers indicate more extensive disease, including larger tumor size and/or spread of the cancer beyond the organ in which it first developed to nearby lymph nodes and/or tissues or organs adjacent to the location of the primary tumor; and (iii) Stage IV, wherein the cancer has spread to distant tissues or organs.

A “solid tumor” is a tumor that is detectable on the basis of tumor mass; e.g., by procedures such as CAT scan, MR imaging, X-ray, ultrasound or palpation, and/or which is detectable because of the expression of one or more cancer-specific antigens in a sample obtainable from a patient. The tumor does not need to have measurable dimensions.

A “subject” to be treated by the methods of the invention can mean either a human or non-human animal, preferably a mammal, more preferably a human. In some embodiments, the subject is a non-human mammal. In some embodiments, the non-human mammal is a non-human primate (e.g., monkeys, apes), ungulate (e.g., cattle, buffalo, sheep, goat, pig, camel, llama, alpaca, deer, horses, donkeys), carnivore (e.g., dog, cat), rodent (e.g., rat, mouse), or lagomorph (e.g., rabbit). In certain embodiments, a subject has a detectable or diagnosed cancer prior to initiation of treatments using the methods of the invention. In certain embodiments, a subject has a detectable or diagnosed infection, e.g., chronic infection, prior to initiation of treatments using the methods of the invention. A “suicide gene” as used herein refers to a gene encoding a protein (e.g., an enzyme) that converts a nontoxic precursor of a drug into a cytotoxic compound.

“Cell turnover”, as used herein, refers to a dynamic process that reorders and disseminates the material within a cell and may ultimately result in cell death. Cell turnover includes the production and release from the cell of cell turnover factors. In some embodiments, cell turnover does not result in cell death.

“Cell turnover factors” or “CTFs”, as used herein, are molecules and cell fragments produced by a cell undergoing cell turnover that are ultimately released from the cell and influence the biological activity of other cells. Cell turnover factors can include proteins, peptides, carbohydrates, lipids, nucleic acids, small molecules, and cell fragments (e.g. vesicles and cell membrane fragments).

A “cell turnover pathway gene”, as used herein, refers to a gene encoding a polypeptide that promotes, induces, or otherwise contributes to a cell turnover pathway.

“Thanotransmission”, as used herein, is communication between cells that is a result of activation of a cell turnover pathway in a target signaling cell, which signals a responding cell to undergo a biological response. Thanotransmission may be induced in a target signaling cell by modulation of cell turnover pathway genes in said cell through, for example, viral or other gene therapy delivery to the target signaling cell of genes that promote such pathways. Table 1 describes exemplary polynucleotides or polypeptides capable of promoting various cell turnover pathways. The target signaling cell in which a cell turnover pathway has been thus activated may signal a responding cell through factors actively released by the signaling cell, or through intracellular factors of the signaling cell that become exposed to the responding cell during the cell turnover (e.g., cell death) of the signaling cell. In certain embodiments, the activated signaling cell promotes an immuno-stimulatory response (e.g., a pro-inflammatory response) in a responding cell (e.g., an immune cell).

“Immuno-modulatory thanotransmission” as used herein refers to thanotransmission in which the activated signaling cell promotes an immuno-modulatory response (e.g. a pro-inflammatory response) in a responding cell (e.g. an immune cell).

The term “thanotransmission polypeptide” as used herein refers to a polypeptide selected from the group consisting of TRIF, RIPK3, Gasdermin E, vICA, Npro, A238L, viral mixed lineage kinase domain like pseudokinase (vMLKL), and variants thereof, and a dominant negative variant of IKBa. In some embodiments, expression of the thanotransmission polypeptide in a target cell results in an increase in immuno-modulatory thanotransmission by the target cell. The terms “polypeptide that promotes thanotransmission” and “thanotransmission polypeptide” are used herein interchangeably. In some embodiments, a thanotransmission polypeptide comprises or consists of TRIF. In some embodiments, a thanotransmission polypeptide comprises or consists of Gasdermin E. In some embodiments, a thanotransmission polypeptide comprises or consists of a fusion of TRIF and Gardermin E polypeptides. In other embodiments, a thanotransmisison polypeptide comprises or consists of a mixture of TRIF and Gasdermin E polypeptides.

In certain aspects, the disclosure relates to a polynucleotide that encodes one or more thanotransmission polypeptides. The terms “polynucleotide that encodes one or more thanotransmissiton polypeptides”, “polynucleotide that encodes a thanotransmission polypeptide”, and “genetic switch” are used herein interchangeably.

The term “recombinant nucleic acid molecule” as used herein refers to a nucleic acid molecule that is prepared by combining two or more polynucleotides to form a nucleic acid molecule that is not found in nature. Accordingly, a recombinant nucleic acid molecule comprises at least two polynucleotides that are covalently bound to a nucleic acid sequence to which they are not covalently bound in nature. For example, in some embodiments, a recombinant nucleic acid molecule comprises two or more polynucleotides, each encoding a different thanotransmission polypeptide, wherein the two or more polynucleotides are covalently bound to a nucleic acid sequence to which they are not covalently bound in nature. In some embodiments, the recombinant nucleic acid molecule comprises at least two polynucleotides that are not found within the same nucleic acid molecule in nature.

“Therapeutically effective amount” means the amount of a compound that, when administered to a patient for treating a disease, is sufficient to effect such treatment for the disease. When administered for preventing a disease, the amount is sufficient to avoid or delay onset of the disease. The “therapeutically effective amount” will vary depending on the compound, the disease and its severity and the age, weight, etc., of the patient to be treated. A therapeutically effective amount need not be curative. A therapeutically effective amount need not prevent a disease or condition from ever occurring. Instead, a therapeutically effective amount is an amount that will at least delay or reduce the onset, severity, or progression of a disease or condition.

As used herein, “treatment”, “treating” and cognates thereof refer to the medical management of a subject with the intent to improve, ameliorate, stabilize, prevent or cure a disease, pathological condition, or disorder. This term includes active treatment (treatment directed to improve the disease, pathological condition, or disorder), causal treatment (treatment directed to the cause of the associated disease, pathological condition, or disorder), palliative treatment (treatment designed for the relief of symptoms), preventative treatment (treatment directed to minimizing or partially or completely inhibiting the development of the associated disease, pathological condition, or disorder); and supportive treatment (treatment employed to supplement another therapy).

The term “variant” as used herein with reference to a polypeptide refers to a polypeptide that differs by at least one amino acid residue from a corresponding wild type polypeptide. In some embodiments, the variant polypeptide has at least one activity that differs from the corresponding naturally occurring polypeptide. The term “variant” as used herein with reference to a polynucleotide refers to a polynucleotide that differs by at least one nucleotide from a corresponding wild type polynucleotide. In some embodiments, a variant polypeptide or variant polynucleotide has at least 50%, 55%, 60%, 65%, 70%, 75%, 80%, 85%, 90%, 95%, 96%, 97%, 98%, or 99% sequence identity to the corresponding wild type polypeptide or polynucleotide and differs by at least one amino acid residue or at least one nucleotide. In some embodiments, the variant is a functional fragment of a polypeptide.

The term “dominant negative variant” as used herein refers to a mutant form of a wildtype protein that reduces or abolishes the activity of the wildtype protein. In some embodiments, the wildtype protein forms a homomeric complex, and the dominant negative variant reduces or abolishes the activity of the complex.

The term “functional fragment” as used herein with reference to a polypeptide refers to a portion of a polypeptide that retains at least one biological activity of the polypeptide, e.g. the ability to promote thanotransmission. In some embodiments, the functional fragment is a domain of the polypeptide, e.g. a death fold domain, a death domain, a pyrin domain, a Death Effector Domain (DED), or a C-terminal caspase recruitment domain (CARD) of the polypeptide. In some embodiments, a functional fragment of a polypeptide is a portion of a domain that retains at least one biological activity of the domain.

A “5′ untranslated region” (5′UTR) as used herein refers to a region of an mRNA that is directly upstream (i.e., 5′) from the start codon (i.e., the first codon of an mRNA transcript translated by a ribosome) that does not encode a polypeptide.

A “3′ untranslated region” (3′UTR) refers to a region of an mRNA that is directly downstream (i.e., 3′) from the stop codon (i.e., the codon of an mRNA transcript that signals a termination of translation) that does not encode a polypeptide.

An “open reading frame” (ORF) is a continuous stretch of DNA or RNA beginning with a start codon (e.g., methionine (ATG)), and ending with a stop codon (e.g., TAA, TAG or TGA) and encoding a polypeptide.

A “polyA tail” is a region of mRNA that is downstream, e.g., directly downstream (i.e., 3′), from the 3′ UTR that contains multiple, consecutive adenosine monophosphates. A polyA tail may contain 10 to 300 adenosine monophosphates. For example, a polyA tail may contain 10, 20, 30, 40, 50, 60, 70, 80, 90, 100, 110, 120, 130, 140, 150, 160, 170, 180, 190, 200, 210, 220, 230, 240, 250, 260, 270, 280, 290 or 300 adenosine monophosphates. In some embodiments, a polyA tail contains 50 to 250 adenosine monophosphates. In a relevant biological setting (e.g., in cells, in vivo) the poly(A) tail functions to protect mRNA from enzymatic degradation, e.g., in the cytoplasm, and aids in transcription termination, export of the mRNA from the nucleus and translation.

The term “pseudotyped virus” as used herein refers to a viral particle formed with a structural and enzymatic core from one virus and the envelope glycoprotein of another virus. In some embodiments, the pseudotyped virus is replication defective. In some embodiments, the pseudotyped virus is replication competent.

### II. Cell Turnover Pathways

The nucleic acid molecules encoding thanotransmission polypeptides, as provided herein, may be used to modulate cell turnover pathways in a target cell. For example, in some embodiments, expression of the nucleic acid molecules and encoded polypeptides in a target cell induces an immuno-stimulatory cell turnover pathway in the target cell. Immuno-stimulatory cell turnover pathways are cell turnover pathways that, when activated in a cell, promote an immune-stimulatory response in a responding cell, such as an immune cell. Immuno-stimulatory cell turnover pathways include, but are not limited to, programmed necrosis (e.g., pyroptosis and necroptosis), extrinsic apoptosis, and combinations thereof.

### Programmed Necrosis

“Programmed necrosis” as used herein refers to a genetically controlled cell death with morphological features such as cellular swelling (oncosis), membrane rupture, and release of cellular contents, in contrast to the retention of membrane integrity that occurs during apoptosis. In some embodiments, the programmed necrosis is pyroptosis. In some embodiments, the programmed necrosis is necroptosis.

Pyroptosis

“Pyroptosis” as used herein refers to the inherently inflammatory process of caspase 1-, caspase 4-, or caspase 5-dependent programmed cell death. The most distinctive biochemical feature of pyroptosis is the early, induced proximity-mediated activation of caspase-1. The pyroptotic activation of caspase-1, 4 or 5 can occur in the context of a multiprotein platform known as the inflammasome, which involves NOD-like receptors (NLRs) or other sensors such as the cytosolic DNA sensor absent in melanoma 2 (AIM2) that recruit the adaptor protein ASC that promotes caspase-1 activation. Caspases-4/5 may be directly activated by LPS. In both cases, active caspase-1 catalyzes the proteolytic maturation and release of pyrogenic interleukin-1β (IL-1β) and IL-18. Moreover, in some (but not all) instances, caspase activation induces cleavage and activation of the pore forming protein GSDM-D to drive membrane rupture and cell death. See Galluzzi et al., 2018, Cell Death Differ. March; 25(3): 486-541. In the methods of the present disclosure, pyroptosis may be induced in a target cell through contact or infection with a virus engineered to comprise one or more polynucleotides encoding a polypeptides that induces pyroptosis in the target cell. Polypeptides that may induce pyroptosis in a target cell include, but are not limited to, NLRs, ASC, GSDM-D, AIM2, and BIRC1.

Several methods are known in the art and may be employed for identifying cells undergoing pyroptosis and distinguishing from other types of cellular disassembly and/or cell death through detection of particular markers. Pyroptosis requires caspase-1, caspase-4, or caspase-5 activity and is usually accompanied by the processing of the pro-IL-lb and/or pro-IL-18, release of these mature cytokines, and membrane permeabilization by a caspase-1/4/5 cleavage fragment of GSDM-D.

Necroptosis

The term “necroptosis” as used herein refers to Receptor interacting protein kinase 1 and/or 3 (RIPK1- and/or RIPK3)/Mixed lineage kinase-like (MLKL)-dependent necrosis. Several triggers can induce necroptosis, including alkylating DNA damage, excitotoxins and the ligation of death receptors. For example, when caspases (and in particular caspase-8 or caspase-10) are inhibited by genetic manipulations (e.g., by gene knockout or RNA interference, RNAi) or blocked by pharmacological agents (e.g., chemical caspase inhibitors), RIPK3 phosphorylates MLKL leading to MLKL assembly into a membrane pore that ultimately activates the execution of necrotic cell death. See Galluzzi et al., 2018, Cell Death Differ. March; 25(3): 486-541, incorporated by reference herein in its entirety.

The same pathways that drive immunogenic apoptosis can activate RIPK3 but normally caspase 8 (and potentially caspase 10) suppresses RIPK3 activation. RIPK3 is typically only activated in situations of caspase 8 compromise. Viral proteins such as vICA or cellular mutants such as FADD dominant negative (DN) target caspase 8 pathways and unleash RIPK3 activity if RIPK3 is present. If RIPK3 is not present, then vICA or FADD-DN simply block apoptosis. Necroptosis is immunogenic because (a) membrane ruptures and (b) an inflammatory transcriptional program (e.g., NF-kB and IRF3) are concomitantly activated.

Necroptosis may be induced in a target cell through expression of thanotransmission polypeptides that induces necroptosis in the target cell. Polypeptides that may induce necroptosis in a target cell include, Toll/interleukin-1 receptor (TIR)-domain-containing adapter-inducing interferon-0 (TRIF), Z-DNA-binding protein 1 (ZBP1), receptor-interacting serine/threonine-protein kinase 1 (RIPK1), receptor-interacting serine/threonine-protein kinase 3 (RIPK3), mixed lineage kinase domain like pseudokinase (MLKL), tumor necrosis factor receptor (TNFR), FS-7-associated surface antigen (FAS), TNF-related apoptosis inducing ligand receptor (TRAILR) and Tumor Necrosis Factor Receptor Type 1-Associated Death Domain Protein (TRADD).

Several methods are known in the art and may be employed for identifying cells undergoing necroptosis and distinguishing from other types of cellular disassembly and/or cell death through detection of particular markers. These include phosphorylation of RIPK1, RIPK3, and MLKL by antibodies that detect these post-translational modifications, typically by immunoblot or immunostaining of cells. Necroptosis can be distinguished from apoptosis and pyroptosis by the absence of caspase activation, rapid membrane permeabilization, MLKL relocalization to membranes, accumulation of RIPK3 and MLKL into detergent insoluble fractions, RIPK3/MLKL complex formation, and MLKL oligomerization. Necroptosis can be genetically and pharmacologically defined by requirement of both RIPK3 and MLKL as well as their activation.

### Extrinsic Apoptosis

The term ‘extrinsic apoptosis’ as used herein refers to instances of apoptotic cell death that are induced by extracellular stress signals which are sensed and propagated by specific transmembrane receptors. Extrinsic apoptosis can be initiated by the binding of ligands, such as FAS/CD95 ligand (FASL/CD95L), tumor necrosis factor α (TNFα), and TNF (ligand) superfamily, member 10 (TNFSF10, best known as TNF-related apoptosis inducing ligand, TRAIL), to various death receptors (i.e., FAS/CD95, TNFα receptor 1 (TNFR1), and TRAIL receptor (TRAILR)1-2, respectively). Alternatively, an extrinsic pro-apoptotic signal can be dispatched by the so-called ‘dependence receptors’, including netrin receptors (e.g., UNC5A-D and deleted in colorectal carcinoma, DCC), which only exert lethal functions when the concentration of their specific ligands falls below a critical threshold level. See Galluzzi et al., 2018, Cell Death Differ. March; 25(3): 486-541, incorporated by reference herein in its entirety.

Extrinsic apoptosis may be induced in a target cell through expression of thanotransmission polypeptides that induce extrinsic apoptosis in the target cell. Polypeptides that may induce extrinsic apoptosis in a target cell include, but are not limited to, TNF, Fas ligand (FasL), TRAIL (and its cognate receptors), TRADD, Fas-associated protein with death domain (FADD), Transforming growth factor beta-activated kinase 1 (Tak1), Caspase-8, XIAP, BID, Caspase-9, APAF-1, CytoC, Caspase-3 and Caspase-7. Polypeptides that may inhibit extrinsic apoptosis in a target cell include Cellular Inhibitor of Apoptosis Protein 1 (cIAP1), cIAP2, Ikka and Ikkb. Several methods are known in the art and may be employed for identifying cells undergoing apoptosis and distinguishing from other types of cellular disassembly and/or cell death through detection of particular markers. Apoptosis requires caspase activation and can be suppressed by inhibitors of caspase activation and/or prevention of death by the absence of caspases such as caspase-8 or caspase-9. Caspase activation systematically dismantles the cell by cleavage of specific substrates such as PARP and DFF45 as well as over 600 additional proteins. Apoptotic cell membranes initially remain intact with externalization of phosphatidyl-serine and concomitant membrane blebbing. Mitochondrial outer membranes are typically disrupted releasing into the cytosol proteins such as CytoC and HTRA2. Nuclear DNA is cleaved into discrete fragments that can be detected by assays known in the art.

### III. Payloads

In certain aspects, the present disclosure relates to recombinant nucleic acid molecules encoding one or more thanotransmission polypeptides (also referred to as genetic switches herein). In certain aspects, the present disclosure relates to combinations of two or more thanotransmission polypeptides, and nucleic molecules encoding these thanotransmission polypeptides. In particular, in some embodiments, the present disclosure relates to the combination of TRIF or a variant thereof with one or more additional thanotransmission polypeptides, e.g., a thanotransmission polypeptide selected from RIPK3, Gasdermin E, vICA, Npro, A238L, vMLKL, and variants thereof, or a dominant negative variant of IKBa (IKBa_DN). For example, in some aspects, the disclosure relates to a recombinant nucleic acid molecule comprising: a) a first polynucleotide encoding TRIF or a variant thereof, and b) a second polynucleotide encoding an additional polypeptide selected from the group consisting of RIPK3, Gasdermin E, vICA, Npro, A238L, vMLKL, and variants thereof, and a dominant negative variant of IKBa.

In some embodiments, the present disclosure relates to the combination of IL-12 or a variant thereof with one or more thanotransmission polypeptides, e.g., a thanotransmission polypeptide selected from TRIF, RIPK3, Gasdermin E, vICA, Npro, A238L, vMLKL, and variants thereof, or a dominant negative variant of IKBa (IKBa_DN). For example, in some aspects, the disclosure relates to a recombinant nucleic acid molecule comprising: a) a first polynucleotide encoding IL-12 or a variant thereof, and b) a second polynucleotide encoding an additional polypeptide selected from the group consisting of TRIF, RIPK3, Gasdermin E, vICA, Npro, A238L, vMLKL, and variants thereof, and a dominant negative variant of IKBa.

The IL-12 and one or more thanotransmission polypeptides may be encoded by a single nucleic acid molecule, or by two or more nucleic acid molecules. For example, in some aspects, the disclosure relates to a recombinant nucleic acid molecule encoding two or more different thanotransmission polypeptides. In some aspects, the disclosure relates to a recombinant nucleic acid molecule encoding IL-12 and one or more thanotransmission polypeptides. In some aspects, the disclosure relates to a combination of two or more recombinant nucleic acid molecules each encoding a different thanotransmission polypeptides. In some aspects, the disclosure relates to a combination of two or more recombinant nucleic acid molecules, wherein one or more of the recombinant nucleic acid molecules encode IL-12, and one or more recombinant nucleic molecules encode one or more different thanotransmission polypeptides.

In some embodiments, the recombinant nucleic acid molecule may encode only one thanotransmission polypeptide, or only IL-12. In some aspects, the disclosure relates to a recombinant nucleic acid molecule comprising a polynucleotide encoding a polypeptide selected from the group consisting of IL-12, TRIF, RIPK3, Gasdermin E, vICA, Npro, A238L, vMLKL, and variants thereof, and a dominant negative variant of IKBa.

In some embodiments, the recombinant nucleic acid molecules encoding IL-12 or the one or more thanotransmission polypeptides are comprised within a pharmaceutical composition, vector, (e.g. an engineered virus, plasmid or transposon) or cell. In some embodiments, the pharmaceutical composition, vector, (e.g. an engineered virus, plasmid or transposon) or cell comprises at least 2, 3, 4 or 5 nucleic acid molecules, each encoding IL-12 or a different thanotransmission polypeptide.

In some embodiments, the recombinant nucleic acid molecule comprises fewer than 100, 90, 80, 70, 60, 50, 40, 30, 20 or 10 kb. In some embodiments, the recombinant nucleic acid molecule comprises at least 10, 20, 30, 40, 50, 60, 70, 80, 90 or 100 kb. Any of these values may be used to define a range for the size of the recombinant nucleic acid molecule. For example, in some embodiments, the recombinant nucleic acid molecule comprises 10-100 kb or 10-50 kb. In some embodiments, the recombinant nucleic acid molecule encodes at least 2, 3, 4, 5, 6, 7, 8, 9 or 10 thanotransmission polypeptides. In some embodiments, the recombinant nucleic acid molecule encodes fewer than 10, 9, 8, 7, 6, 5, 4 or 3 thanotransmission polypeptides. Any of these values may be used to define a range for the number of thanotransmission polypeptides encoded by the recombinant nucleic acid molecule. For example, in some embodiments, the recombinant nucleic acid molecule encodes 2-3, 2-4 or 2-10 thanotransmission polypeptides. In some embodiments, the recombinant nucleic acid molecule encodes only two thanotransmission polypeptides. In some embodiments, the recombinant nucleic acid molecule encodes only three thanotransmission polypeptides.

Exemplary polynucleotide sequences encoding the thanotransmission polypeptides and IL-12 are provided in Table 1 below. Any other polynucleotide sequences that encode the thanotransmission polypeptides and IL-12 of Table 1 (or encode polypeptides at least 85%, 87%, 90%, 95%, 97%, 98%, or 99% identical thereto) can also be used in the methods and compositions described herein. In some embodiments, the thanotransmission polypeptide or IL-12 is a wild type protein, or a functional fragment thereof. In some embodiments, the functional fragment is an N-terminal or C-terminal truncation of a wild type protein, e.g., a wildtype thanotransmission polypeptide as described herein. In some embodiments, the IL-12 or thanotransmission polypeptides described herein may be mutated, for example, to further enhance their ability to promote thanotransmission. For example, in some embodiments, the IL-12 or thanotransmission polypeptide or functional fragment thereof comprises one or more mutations relative to the wild type protein.

TABLE 1
Polynucleotide sequences encoding IL-12 or thanotransmission polypeptides. For the
TRIF_180, IKBa_DN and Casp1FV variants, the Uniprot accession number, Gene ID, and
Entrez Gene ID for the wildtype human protein are provided. DN = dominant negative; WT = wildtype.

| Polypeptide | Gene name | Uniprot | Gene ID | Entrez Gene ID | Notes |
|---|---|---|---|---|---|
| TRIF | TICAM1 | Q8IUC6 | 148022 | ENSG00000127666 | Full length WT human |
| TRIF_180 variant | TICAM1 | Q8IUC6 | 148022 | ENSG00000127666 | human TRIF, deletion |
| | | | | | of aa1-180 |
| N-terminal | NA | P19712 | NA | NA | Encodes AA 1-168 |
| protease of | | | | | of viral protein |
| viral protein | | | | | |
| (Npro) | | | | | |
| A238L | A238L | O36972 | AF014479 | NA | Full length WT viral |
| GSDME | GSDME | O60443 | 1687 | ENSG00000105928 | Full length WT human |
| vMLKL | BAV00186 | A0A1L3IZS3 | NA | KY094066 | Full length, WT viral |
| RIPK3 | RIPK3 | Q9Y572 | 11035 | ENSG00000129465 | Full length WT human |
| vICA | UL36 | F5HAY6 | NA | AF014479 | Full length WT viral |
| IKBa_DN variant | NFKBIA | P25963 | 4792 | ENSG00000100906 | Encodes DN variant |
| | | | | | of human protein; |
| | | | | | S32A, S36A |
| Casp1FV variant | CASP1 | P29466 | 834 | ENSG00000137752 | N-terminal fragment |
| | | | | | of human WT, with a |
| | | | | | self-dimerizing domain |
| | | | | | added to C-terminus |
| IL-12 p35 subunit | IL-12A | P29459 | 3592 | NA | Full length WT human |
| IL-12 p40 subunit | IL-12B | P29460 | 3593 | NA | Full length WT human |

The IL-12 and two or more thanotransmission polypeptides may be expressed as separate polypeptides, or they may be comprised within a fusion protein. In some embodiments, at least one of the polynucleotides that encodes IL-12 or a thanotransmission polypeptide is transcribed as a single transcript that encodes the two or more thanotransmission polypeptides. In some embodiments, this single transcript encoding the two or more thanotransmission polypeptides is translated as a single polypeptide (e.g., a fusion protein) comprising the two or more thanotransmission polypeptides. In some embodiments, this single transcript encoding the two or more thanotransmission polypeptides is translated as separate thanotransmission polypeptides, for example, through the inclusion of a 2A peptide (e.g., a P2A peptide) as described herein separating the thanotransmission polypeptides.

The thanotransmission polypeptides described herein may promote thanotransmission through various mechanisms, including but not limited to activation of NF-κB, activation of IRF3 and/or IRF7, promotion of apoptosis, and promotion of programmed necrosis (e.g., necroptosis or pyroptosis). When combinations of two or more thanotransmission polypeptides are used, each of the two or more thanotransmission polypeptides may promote thanotransmission through similar mechanisms, or through different mechanisms. For example, in some embodiments, at least two of the thanotransmission polypeptides encoded by the one or more polynucleotides activate NF-κB. In some embodiments, at least two of the thanotransmission polypeptides encoded by the one or more polynucleotides activate IRF3 and/or IRF7. In some embodiments, at least two of the thanotransmission polypeptides encoded by the one or more polynucleotides promote apoptosis. In some embodiments, at least two of the thanotransmission polypeptides encoded by the one or more polynucleotides promote programmed necrosis (e.g., necroptosis or pyroptosis).

When the two or more thanotransmission polypeptides promote thanotransmission through different mechanisms, various combinations of mechanisms may be used. For example, in some embodiments, at least one of the thanotransmission polypeptides encoded by the one or more thanotransmission polynucleotides activates NF-κB, and at least one of the thanotransmission polypeptides encoded by the one or more polynucleotides activates IRF3 and/or IRF7. In some embodiments, at least one of the thanotransmission polypeptides encoded by the one or more polynucleotides activates NF-κB, and at least one of the thanotransmission polypeptides encoded by the one or more polynucleotides promotes apoptosis. In some embodiments, at least one of the thanotransmission polypeptides encoded by the one or more polynucleotides activates NF-κB, and at least one of the thanotransmission polypeptides encoded by the one or more polynucleotides promotes programmed necrosis (e.g., necroptosis or pyroptosis). In some embodiments, at least one of the thanotransmission polypeptides encoded by the one or more polynucleotides activates IRF3 and/or IRF7, and at least one of the thanotransmission polypeptides encoded by the one or more polynucleotides promotes apoptosis. In some embodiments, at least one of the thanotransmission polypeptides encoded by the one or more thanotransmission polynucleotides activates IRF3 and/or IRF7, and at least one of the thanotransmission polypeptides encoded by the one or more polynucleotides promotes programmed necrosis (e.g., necroptosis or pyroptosis). In some embodiments, at least one of the thanotransmission polypeptides encoded by the one or more polynucleotides promotes apoptosis, and at least one of the thanotransmission polypeptides encoded by the one or more thanotransmission polynucleotides promotes programmed necrosis (e.g., necroptosis or pyroptosis).

In a particular embodiment, at least one of the thanotransmission polypeptides is TRIF or a variant (e.g., functional fragment) thereof. In some embodiments, the thanotransmission polypeptide comprises of consists of SEQ ID NO: 2. In some embodiments, the thanotransmission polypeptide comprises or consists of a polypeptide having at least 85%, 87%, 90%, 95%, 96%, 97%, 98% or 99% sequence identity to SEQ ID NO: 2. In some embodiments, the thanotransmission polypeptide is encoded by a polynucleotide comprising or consisting of SEQ ID NO: 1. In some embodiments, the thanotransmission polypeptide is encoded by a polynucleotide having at least 85%, 87%, 90%, 95%, 96%, 97%, 98% or 99% sequence identity to SEQ ID NO: 1.

TRIF Variants

In some embodiments, the thanotransmission polypeptide is a variant of a TRIF protein, e.g., a variant of the wildtype human TRIF protein of SEQ ID NO: 2. In some embodiments, the TRIF variant comprises a mutation in one or more amino acid residues of a RHIM tetrad at positions 688 to 691 of the wildtype human TRIF protein of SEQ ID NO: 2. In some embodiments, the TRIF variant comprises one or more substitutions selected from the group consisting of Q688A, L689A, G690A and L691A relative to the wildtype human TRIF protein of SEQ ID NO: 2. In some embodiments, the TRIF variant comprises the substitutions Q688A, L689A, G690A and L691A relative to the wildtype human TRIF protein of SEQ ID NO: 2.

In some embodiments, the TRIF variant comprises a deletion of one or more amino acid residues at the C-terminus relative to the corresponding wildtype TRIF protein, e.g., relative to the human wildtype TRIF protein. In some embodiments, the TRIF variant is a variant of the wildtype human TRIF protein comprising a deletion of the amino acid residues at positions 541-712 of SEQ ID NO: 2. In some embodiments, the TRIF variant is a human TRIF variant comprising a deletion of the amino acid residues at positions 546-712 of the wildtype human TRIF protein of SEQ ID NO: 2.

In some embodiments, the TRIF variant comprises a mutation of one or more of the amino acid residues that are phosphorylated by TBK1. In some embodiments, the TRIF variant comprises one or more substitutions selected from the group consisting of S210A, S212A and T214A relative to the wildtype human TRIF protein of SEQ ID NO: 2.

In some embodiments, the TRIF variant comprises a mutation of the amino acid residue at position 434 relative to the wildtype human TRIF protein of SEQ ID NO: 2. In some embodiments, the TRIF variant comprises a P434H substitution relative to the wildtype human TRIF protein of SEQ ID NO: 2.

In some embodiments, the TRIF variant comprises a deletion of one or more amino acid residues at the N-terminus relative to the corresponding wildtype TRIF protein, e.g., relative to the human wildtype TRIF protein. In some embodiments, the TRIF variant is a human TRIF variant comprising a deletion of the amino acid residues at positions 1-311 of the wildtype human TRIF protein of SEQ ID NO: 2. In some embodiments, the TRIF variant consists of SEQ ID NO: 12, or a polypeptide having at least 85%, 87%, 90%, 95%, 96%, 97%, 98%, or 99% sequence identity to SEQ ID NO: 12. In some embodiments, the TRIF variant is a human TRIF variant comprising a deletion of the amino acid residues at positions 1-180 of the wildtype human TRIF protein of SEQ ID NO: 2.

In some embodiments, the TRIF variant is a human TRIF variant comprising a deletion of the amino acid residues at positions 217-658 of the wildtype human TRIF protein of SEQ ID NO: 2. In some embodiments, the TRIF variant is a human TRIF variant comprising a deletion of the amino acid residues at positions 217-386 of the wildtype human TRIF protein of SEQ ID NO: 2. In some embodiments, the TRIF variant is a human TRIF variant comprising a deletion of the amino acid residues at positions 1-180 and 217-658 of the wildtype human TRIF protein of SEQ ID NO: 2. In some embodiments, the TRIF variant is a human TRIF variant comprising a deletion of the amino acid residues at positions 1-180, 217-386 and 546-712 of the wildtype human TRIF protein of SEQ ID NO: 2.

In some embodiments, the TRIF variant comprises SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20 or SEQ ID NO: 22, or a polypeptide having at least 85%, 87%, 90%, 95%, 96%, 97%, 98%, or 99% sequence identity to SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20 or SEQ ID NO: 22.

In some embodiments, the TRIF variant consists of SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20 or SEQ ID NO: 22. In some embodiments, the TRIF variant consists of a polypeptide having at least 85%, 87%, 90%, 95%, 96%, 97%, 98%, or 99% sequence identity to SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20 or SEQ ID NO: 22.

In some embodiments, the TRIF variant is encoded by a polynucleotide comprising SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19 or SEQ ID NO: 21. In some embodiments, the TRIF variant is encoded by a polynucleotide comprising a nucleic acid sequence having at least 85%, 87%, 90%, 95%, 96%, 97%, 98%, or 99% sequence identity to SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19 or SEQ ID NO: 21.

In some embodiments, the TRIF variant is encoded by a polynucleotide consisting of SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19 or SEQ ID NO: 21. In some embodiments, the TRIF variant is encoded by a polynucleotide consisting of a nucleic acid sequence having at least 85%, 87%, 90%, 95%, 96%, 97%, 98%, or 99% sequence identity to SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20 or SEQ ID NO: 22.

In a particular embodiment, at least one of the thanotransmission polypeptides is RIPK3 or a variant (e.g., functional fragment) thereof. In some embodiments, the thanotransmission polypeptide comprises of consists of SEQ ID NO: 30. In some embodiments, the thanotransmission polypeptide comprises or consists of a polypeptide having at least 85%, 87%, 90%, 95%, 96%, 97%, 98% or 99% sequence identity to SEQ ID NO: 30. In some embodiments, the thanotransmission polypeptide is encoded by a polynucleotide comprising or consisting of SEQ ID NO: 31. In some embodiments, the thanotransmission polypeptide is encoded by a polynucleotide having at least 85%, 87%, 90%, 95%, 96%, 97%, 98% or 99% sequence identity to SEQ ID NO: 31.

In a particular embodiment, at least one of the thanotransmission polypeptides is TRIF or a functional fragment or variant thereof, and at least one of the thanotransmission polypeptides is RIPK3 or a variant (e.g., functional fragment) thereof.

In a particular embodiment, the thanotransmission polypeptide is vICA. The vICA protein is a human cytomegalovirus (CMV) protein encoded by the UL36 gene. See Skaletskaya et al., PNAS Jul. 3, 2001 98 (14) 7829-7834, which is incorporated by reference herein in its entirety. The vICA protein inhibits Fas-mediated apoptosis by binding to the pro-domain of caspase-8 and preventing its activation. In some embodiments, the vICA protein comprises or consists of SEQ ID NO: 32. In some embodiments, the vICA protein comprises or consists of an amino acid sequence having at least 85%, 87%, 90%, 95%, 96%, 97%, 98% or 99% sequence identity to SEQ ID NO: 32. In some embodiments, a recombinant nucleic acid molecule as described herein comprise a polynucleotide encoding SEQ ID NO: 32. In some embodiments, a recombinant nucleic acid molecule as described herein comprise a polynucleotide encoding an amino acid sequence having at least 85%, 87%, 90%, 95%, 96%, 97%, 98% or 99% sequence identity to SEQ ID NO: 32.

In some embodiments, at least one of the thanotransmission polypeptides is TRIF or a variant (e.g., functional fragment) thereof, at least one of the thanotransmission polypeptides is RIPK3 or a variant (e.g., functional fragment) thereof, and at least one of the thanotransmission polypeptides is vICA or a variant (e.g., functional fragment) thereof.

The gasdermins are a family of pore-forming effector proteins that cause membrane permeabilization and pyroptosis. The gasdermin proteins include Gasdermin A, Gasdermin B, Gasdermin C, Gasdermin D and Gasdermin E. Gasdermins contain a cytotoxic N-terminal domain and a C-terminal repressor domain connected by a flexible linker. Proteolytic cleavage between these two domains releases the intramolecular inhibition on the cytotoxic domain, allowing it to insert into cell membranes and form large oligomeric pores, which disrupts ion homeostasis and induces cell death. See Broz et al., 2020, Nature Reviews Immunology 20: 143-157, which is incorporated by reference herein in its entirety. For example, Gasdermin E (GSDME, also known as DFNA5) can be cleaved by caspase 3, thereby converting noninflammatory apoptosis to pyroptosis in GSDME-expressing cells. Similarly, caspases 1, 4 and 5 cleave and activate Gasdermin D. In some embodiments, the variant of Gasermin E is an N-terminal domain of Gasdermin E.

In some embodiments, at least one of the thanotransmission polypeptides is Gasdermin E or a variant (e.g., functional fragment) thereof. In some embodiments, the thanotransmission polypeptide comprises of consists of SEQ ID NO: 45. In some embodiments, the thanotransmission polypeptide comprises or consists of a polypeptide having at least 85%, 87%, 90%, 95%, 96%, 97%, 98% or 99% sequence identity to SEQ ID NO: 45. In some embodiments, the thanotransmission polypeptide is encoded by a polynucleotide comprising or consisting of SEQ ID NO: 44. In some embodiments, the thanotransmission polypeptide is encoded by a polynucleotide having at least 85%, 87%, 90%, 95%, 96%, 97%, 98% or 99% sequence identity to SEQ ID NO: 44.

In some embodiments, at least one of the thanotransmission polypeptides is TRIF or a variant (e.g., functional fragment) thereof, and at least one of the thanotransmission polypeptides is Gasdermin E or a variant (e.g., functional fragment) thereof.

In some embodiments, at least one of the thanotransmission polypeptides is TRIF or a variant (e.g., functional fragment) thereof, at least one of the thanotransmission polypeptides is RIPK3 or a variant (e.g., functional fragment) thereof, and at least one of the thanotransmission polypeptides is Gasdermin E or a variant (e.g., functional fragment) thereof.

In some embodiments, at least one of the thanotransmission polypeptides is Npro or a variant (e.g., functional fragment) thereof. In some embodiments, the thanotransmission polypeptide is encoded by a polynucleotide comprising or consisting of SEQ ID NO: 45. In some embodiments, the thanotransmission polypeptide is encoded by a polynucleotide having at least 85%, 87%, 90%, 95%, 96%, 97%, 98% or 99% sequence identity to SEQ ID NO: 45.

In some embodiments, at least one of the thanotransmission polypeptides is TRIF or a variant (e.g., functional fragment) thereof, and at least one of the thanotransmission polypeptides is Npro or a variant (e.g., functional fragment) thereof.

In some embodiments, at least one of the thanotransmission polypeptides is A238L or a variant (e.g., functional fragment) thereof. In some embodiments, the thanotransmission polypeptide is encoded by a polynucleotide comprising or consisting of SEQ ID NO: 46. In some embodiments, the thanotransmission polypeptide is encoded by a polynucleotide having at least 85%, 87%, 90%, 95%, 96%, 97%, 98% or 99% sequence identity to SEQ ID NO: 46.

In some embodiments, at least one of the thanotransmission polypeptides is TRIF or a variant (e.g., functional fragment) thereof, and at least one of the thanotransmission polypeptides is A238L or a variant (e.g., functional fragment) thereof.

In some embodiments, at least one of the thanotransmission polypeptides is vMLKL or a variant (e.g., functional fragment) thereof. In some embodiments, the thanotransmission polypeptide is encoded by a polynucleotide comprising or consisting of SEQ ID NO: 47. In some embodiments, the thanotransmission polypeptide is encoded by a polynucleotide having at least 85%, 87%, 90%, 95%, 96%, 97%, 98% or 99% sequence identity to SEQ ID NO: 47.

In some embodiments, at least one of the thanotransmission polypeptides is TRIF or a variant (e.g., functional fragment) thereof, and at least one of the thanotransmission polypeptides is vMLKL or a variant (e.g., functional fragment) thereof.

In some embodiments, at least one of the thanotransmission polypeptides is a dominant negative variant of IKBa. In some embodiments, the dominant negative variant of IKBa comprises an S32A substitution and an S36A substitution relative to the human wildtype IKBa amino acid sequence of SEQ ID NO: 50. In some embodiments, the thanotransmission polypeptide comprises of consists of SEQ ID NO: 50. In some embodiments, the thanotransmission polypeptide comprises or consists of a polypeptide having at least 85%, 87%, 90%, 95%, 96%, 97%, 98% or 99% sequence identity to SEQ ID NO: 50. In some embodiments, the thanotransmission polypeptide is encoded by a polynucleotide comprising or consisting of SEQ ID NO: 49. In some embodiments, the thanotransmission polypeptide is encoded by a polynucleotide having at least 85%, 87%, 90%, 95%, 96%, 97%, 98% or 99% sequence identity to SEQ ID NO: 49.

In some embodiments, at least one of the thanotransmission polypeptides is TRIF or a variant (e.g., functional fragment) thereof, and at least one of the thanotransmission polypeptides is a dominant negative variant of IKBa as described herein.

In some embodiments, one or more genetic switches are used in combination with a nucleic acid encoding an immunomodulatory cytokine, e.g., IL-12 or a variant (e.g., functional fragment) thereof. In some embodiments, at least one of the polypeptides is IL-12 or a variant (e.g., functional fragment) thereof. In some embodiments, the IL-12 polypeptide comprises SEQ ID NO: 60 (p35 IL-12 subunit) and SEQ ID NO: 62 (p40 IL-12 subunit). In some embodiments, the IL-12 polypeptide consists of SEQ ID NO: 60 (p35 IL-12 subunit) and SEQ ID NO: 62 (p40 IL-12 subunit). In some embodiments, the IL-12 polypeptide comprises a polypeptide having at least 85%, 87%, 90%, 95%, 96%, 97%, 98% or 99% sequence identity to SEQ ID NO: 60 and a polypeptide having at least 85%, 87%, 90%, 95%, 96%, 97%, 98% or 99% sequence identity to SEQ ID NO: 62. In some embodiments, the IL-12 polypeptide consists of a polypeptide having at least 85%, 87%, 90%, 95%, 96%, 97%, 98% or 99% sequence identity to SEQ ID NO: 60 and a polypeptide having at least 85%, 87%, 90%, 95%, 96%, 97%, 98% or 99% sequence identity to SEQ ID NO: 62. In some embodiments, the IL-12 polypeptide further comprises a linker between the p35 subunit and the p40 subunit. In some embodiments, the linker comprises or consists of the amino acid sequence of SEQ ID NO: 59.

In some embodiments, the IL-12 polypeptide is encoded by a polynucleotide comprising SEQ ID NO: 61 (p35 IL-12 subunit) and SEQ ID NO: 63 (p40 IL-12 subunit). In some embodiments, the IL-12 polypeptide is encoded by a polynucleotide consisting of SEQ ID NO: 61 and SEQ ID NO: 63. In some embodiments, the IL-12 polypeptide is encoded by a polynucleotide having at least 85%, 87%, 90%, 95%, 96%, 97%, 98% or 99% sequence identity to SEQ ID NO: 61 (p35 IL-12 subunit) and a polynucleotide having at least 85%, 87%, 90%, 95%, 96%, 97%, 98% or 99% sequence identity to SEQ ID NO: 63 (p40 IL-12 subunit). In some embodiments, the polynucleotide encoding the IL-12 polypeptide further comprises a polynucleotide encoding a linker between the polynucleotide encoding the p35 subunit and the polynucleotide encoding the p40 subunit. In some embodiments, the polynucleotide encoding the linker comprises or consists of SEQ ID NO: 58.

In some embodiments, the polynucleotide encoding the p35 subunit of IL-12 (e.g., SEQ ID NO: 61) and the polynucleotide encoding the p40 subunit of IL-12 (e.g., SEQ ID NO: 63) are on the same recombinant nucleic acid molecule. In some embodiments, the polynucleotide encoding the p35 subunit of IL-12 (e.g., SEQ ID NO: 61) and the polynucleotide encoding the p40 subunit of IL-12 (e.g., SEQ ID NO: 63) are on different recombinant nucleic acid molecules.

In some embodiments, at least one of the polypeptides is IL-12 or a variant (e.g., functional fragment) thereof, and at least one of the thanotransmission polypeptides is TRIF or a variant (e.g., functional fragment) thereof.

In some embodiments, at least one of the polypeptides is IL-12 or a variant (e.g., functional fragment) thereof, and at least one of the thanotransmission polypeptides is Gasdermin E or a variant (e.g., functional fragment) thereof.

In some embodiments, at least one of the polypeptides is IL-12 or a variant (e.g., functional fragment) thereof, at least one of the thanotransmission polypeptides is TRIF or a variant (e.g., functional fragment) thereof, and at least one of the thanotransmission polypeptides is Gasdermin E or a variant (e.g., functional fragment) thereof.

Fusion Proteins

In some embodiments, the recombinant nucleic acid molecule encoding two or more different thanotransmission polypeptides may encode a fusion protein. In some embodiments, the recombinant nucleic acid molecule encoding IL-12 and one or more different thanotransmission polypeptides may encode a fusion protein. In some embodiments, the fusion protein may comprise any two or more different thanotransmission polypeptides as disclosed in Table 1 above, or variants (e.g., functional fragments) thereof. In some embodiments, the fusion protein may comprise IL-12 and any one or more thanotransmission polypeptides as disclosed in Table 1 above, or variants (e.g., functional fragments) thereof. In some embodiments, the functional fragment is a domain of the thanotransmission polypeptide, e.g., a RHIM domain, death domain (DD), death effector domain (DED), Caspase Recruitment Domain (CARD), Large subunit/Small subunit (L/S) domain, RIPK-derived kinase domain, or Toll/interleukin-1 receptor (TIR)-domain.

In some embodiments, the fusion protein comprises TRIF or a variant thereof. In some embodiments, the fusion protein comprises RIPK3 or a variant thereof. In some embodiments, the fusion protein comprises TRIF or a variant thereof and RIPK3 or a variant thereof. In some embodiments, the fusion protein comprises the amino acid sequence of SEQ ID NO: 12, or an amino acid sequence having at least 85%, 87%, 90%, 95%, 96%, 97%, 98% or 99% sequence identity to SEQ ID NO: 12. In some embodiments, the fusion protein comprises the amino acid sequence of SEQ ID NO: 22, or an amino acid sequence having at least 85%, 87%, 90%, 95%, 96%, 97%, 98% or 99% sequence identity to SEQ ID NO: 22.

In some embodiments, the fusion protein further comprises one or more linkers, e.g., one or more linkers located between the thanotransmission polypeptides that comprise the fusion protein. In some embodiments, the linker comprises or consists of SEQ ID NO: 25.

In some embodiments, the two subunits of IL-12, i.e., the p35 and p40 subunits are comprised within a fusion protein. In some embodiments, the IL-12 polypeptide described herein comprises the p35 and p40 subunits directly fused to each other, i.e., without an intervening linker. In some embodiments, the IL-12 polypeptide is a fusion protein comprising the p35 subunit, the p40 subunit, and a linker located between the p35 subunit and the p40 subunit. In some embodiments, the linker comprises or consists of the amino acid sequence of SEQ ID NO: 59.

Linkers

Linker peptides are included as spacers between two polypeptides, e.g., the p35 and p40 subunits of IL-12. Linker peptides can promote proper protein folding and stability of the component polypeptides, improve protein expression, and enable better bioactivity of the component polypeptides (Chen, et al., 2013, Adv Drug Deliv Rev. 65:1357-69). Peptide linkers used fusion proteins may be unstructured flexible peptides or structured rigid peptides. A study of the length, sequence, and conformation of linkers peptides between independent structural domains in natural proteins has provided a theoretical basis for the design of flexible peptide linkers (Argos, 1990, J Mol Biol. 211:943-58). Argos provided the guidance that long flexible linker peptides be composed of small nonpolar residues like Glycine and small polar resides like Serine and Threonine, with multiple Glycine residues enabling a highly flexible conformation and Serine or Threonine providing polar surface area to limit hydrophobic interaction within the peptide or with the component fusion protein moieties. Many peptide linkers described in the literature are rich in glycine and serine, such as repeats of the sequence GGGGS (SEQ ID NO: 67), although an artisan skilled in the art will recognize that other sequences following the general recommendations of Argos (Argos, 1990, J Mol Biol. 20; 211(4):943-58) can also be used.

Flexible linker peptides can be composed of glycine, serine, threonine, with multiple glycine residues providing a highly flexible conformation. Serine or threonine residues provide polar surface area to limit hydrophobic interaction within the peptide or with the component fusion protein moieties. In some embodiments, peptide linkers are rich in glycine and serine, such as repeats of the sequence GGGGS (SEQ ID NO: 67). In some embodiments, the linker comprises or consists of glycine and serine residues. In some embodiments, a peptide linker has a sequence of (GGGGS)n, wherein n is 1, 2, 3, 4, 5, 6, 7, 8, 9, or 10. In some embodiments, n is 3; i.e., a peptide linker has a sequence of GGGGSGGGGSGGGGS (SEQ ID NO: 59). In some embodiments the peptide linker comprises an amino acid sequence having at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the amino acid sequence of SEQ ID NO: 59.

In some embodiments, the linker is 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90 or 100 amino acid residues in length. In some embodiments, the linker is at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90 or 100 amino acid residues in length. In some embodiments, the linker is less than 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90 or 100 amino acid residues in length. Any of these values may be used to define a range for the length of the linker. For example, in some embodiments, the linker is 5-100, 5-50 or 10-25 amino acid residues in length. In a particular embodiment, the length of the linker is 15 amino acid residues.

In certain aspects, the disclosure relates to a recombinant nucleic acid molecule comprising a polynucleotide encoding a polypeptide selected from the group consisting of IL-12, TRIF, RIPK3, Gasdermin E, vICA, Npro, A238L, vMLKL, and variants thereof, or a dominant negative variant of IKBa.

In certain aspects, the disclosure relates to a recombinant nucleic acid molecule comprising a polynucleotide encoding a TRIF variant as described herein. In some embodiments, the TRIF variant comprises an N-terminal deletion. In some embodiments, the TRIF variant comprises a deletion of 180 amino acid residues at the N-terminus. In some embodiments, the TRIF variant comprises a deletion of 180 amino acid residues at the N-terminus relative to the wildtype human TRIF amino acid sequence of SEQ ID NO: 2. In some embodiments, the TRIF variant consists of the TRIF_d1-180 variant amino acid sequence of SEQ ID NO: 14.

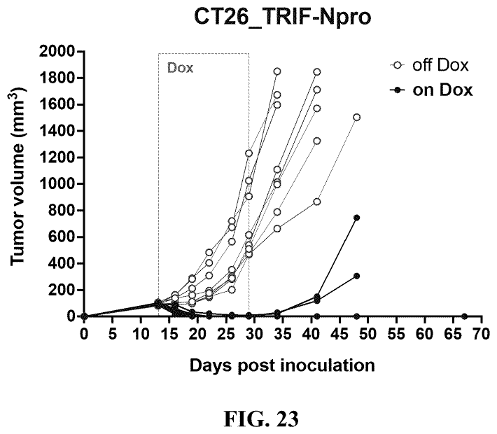

In certain aspects, the disclosure relates to a recombinant nucleic acid molecule comprising a polynucleotide encoding a caspase 1 variant. In some embodiments, the caspase 1 variant comprises a self-dimerization domain. As an initiator caspase, caspase 1 exists as an inactive monomer and requires dimerization to achieve catalytic competence. Accordingly, a self-dimerization domain may be added to caspase 1 for activation of the protein. In some embodiments, the self-dimerization domain is a C-terminal self-dimerization domain, for example, the C-terminal self-dimerization domain shown in FIG. 23. In some embodiments, the caspase 1 variant comprises the amino acid sequence of SEQ ID NO: 54.

In some embodiments, the caspase 1 variant comprises or consists of a polypeptide having at least 85%, 87%, 90%, 95%, 96%, 97%, 98% or 99% sequence identity to the human wildtype caspase 1 amino acid sequence of SEQ ID NO: 52. In some embodiments, the caspase 1 variant is encoded by a polynucleotide having at least 85%, 87%, 90%, 95%, 96%, 97%, 98% or 99% sequence identity to the human wildtype caspase 1 nucleic acid sequence of SEQ ID NO: 51.

Immune Stimulatory Proteins

The recombinant nucleic acid molecule encoding IL-12 and/or one or more thanotransmission polypeptides, or the vector (e.g., virus, plasmid or transposon), cell or pharmaceutical composition, may further comprise one or more polynucleotides encoding an immune stimulatory protein. In one embodiment, the immune stimulatory protein is an antagonist of transforming growth factor beta (TGF-β), a colony-stimulating factor, a cytokine, an immune checkpoint modulator, an flt3 ligand, or an antibody agonist of ft3.

The colony-stimulating factor may be a granulocyte-macrophage colony-stimulating factor (GM-CSF). In one embodiment, the polynucleotide encoding GM-CSF is inserted into the ICP34.5 gene locus.

The cytokine may be an interleukin. In one embodiment, the interleukin is selected from the group consisting of IL-1α, IL-1β, IL-2, IL-4, IL-12, IL-15, IL-18, IL-21, IL-24, IL-33, IL-36α, IL-36β and IL-36γ. Additional suitable cytokines include a type I interferon, interferon gamma, a type III interferon and TNFα. In some embodiments, the interleukin is IL-12.

In some embodiments, the immune checkpoint modulator is an antagonist of an inhibitory immune checkpoint protein. Examples of inhibitory immune checkpoint protein include, but are not limited to, ADORA2A, B7-H3, B7-H4, IDO, KIR, VISTA, PD-1, PD-L1, PD-L2, LAG3, Tim3, BTLA and CTLA4. In some embodiments, the immune checkpoint modulator is an agonist of a stimulatory immune checkpoint protein. Examples of stimulatory immune checkpoint proteins include, but are not limited to, CD27, CD28, CD40, CD122, OX40, GITR, ICOS and 4-1BB. In some embodiments, the agonist of the stimulatory immune checkpoint protein is selected from CD40 ligand (CD40L), ICOS ligand, GITR ligand, 4-1-BB ligand, OX40 Ligand and a modified version of any thereof. In some embodiments, the agonist of the stimulatory immune checkpoint protein is an antibody agonist of a protein selected from CD40, ICOS, GITR, 4-1-BB and 0X40.

Suicide Genes

The recombinant nucleic acid molecule encoding IL-12 and/or one or more thanotransmission polypeptides, or the vector (e.g., engineered virus, plasmid or transposon), cell or pharmaceutical composition, may further comprise a suicide gene. The term “suicide gene” refers to a gene encoding a protein (e.g., an enzyme) that converts a nontoxic precursor of a drug into a cytotoxic compound. In some embodiments, the suicide gene encodes a polypeptide selected from the group consisting of FK506 binding protein (FKBP)-FAS, FKBP-caspase-8, FKBP-caspase-9, a polypeptide having cytosine deaminase (CDase) activity, a polypeptide having thymidine kinase activity, a polypeptide having uracil phosphoribosyl transferase (UPRTase) activity, and a polypeptide having purine nucleoside phosphorylase activity.

In some embodiments, the polypeptide having CDase activity is FCY1, FCA1 or CodA.

In some embodiments, the polypeptide having UPRTase activity is FUR1 or a variant thereof, e.g., FUR1Δ105. FUR1Δ105 is an FUR1 gene lacking the first 105 nucleotides in the 5′ region of the coding region allowing the synthesis of a UPRTase from which the first 35 amino acid residues have been deleted at the N-terminus. FUR1Δ105 starts with the methionine at position 36 of the native protein.

The suicide gene may encode a fusion protein, e.g., a fusion protein having CDase and UPRTase activity. In some embodiments, the fusion protein is selected from codA::upp, FCY1::FUR1, FCY1::FUR1Δ105 (FCU1) and FCU1-8 polypeptides.

2A Peptides

The recombinant nucleic acid molecule encoding IL-12 and/or the one or more thanotransmission polypeptides, or the vector (e.g., engineered virus, plasmid or transposon), cell or pharmaceutical composition, may further comprise a polynucleotide encoding a 2A peptide. 2A peptides induce ribosomal skipping during translation of a protein, such that two proteins encoded by the same mRNA transcript may be expressed as separate proteins. See Liu et al., 2017, Scientific Reports. 7 (1): 2193, which is incorporated by reference herein in its entirety. These peptides share a core sequence motif, are about 18-22 amino acid residues in length, and are found in a wide range of viruses. Exemplary 2A peptides include, but are not limited to T2A, P2A, E2A and F2A. In a particular embodiment, the 2A peptide is a P2A peptide. The polynucleotide encoding the 2A peptide may be located between polynucleotides encoding two different thanotransmission polypeptides to allow for separate expression of each thanotransmission polypeptide. In some embodiments, the recombinant nucleic acid molecule comprises a polynucleotide encoding the T2A peptide of SEQ ID NO: 26. In some embodiments, the nucleic acid molecule comprises a polynucleotide encoding the P2A peptide of SEQ ID NO: 27. In some embodiments, the recombinant nucleic acid molecule comprises a polynucleotide encoding the E2A peptide of SEQ ID NO: 28. In some embodiments, the recombinant nucleic acid molecule comprises a polynucleotide encoding the F2A peptide of SEQ ID NO: 29. The 2A peptide may further comprise a GSG linker at the N-terminus.

In some embodiments the nucleic acid molecule comprises a polynucleotide encoding TRIF, a polynucleotide encoding RIPK3 and a polynucleotide encoding a 2A peptide located between the polynucleotide encoding TRIF and the polynucleotide encoding RIPK3 (e.g., TRIF-2A-RIPK3). In some embodiments the nucleic acid molecule comprises a polynucleotide encoding TRIF, a polynucleotide encoding RIPK3, a polynucleotide encoding vICA, a polynucleotide encoding a 2A peptide located between the polynucleotide encoding TRIF and the polynucleotide encoding RIPK3, and a polynucleotide encoding a 2A peptide between the polynucleotide encoding RIPK3 and the polynucleotide encoding vICA (e.g. TRIF-2A-RIPK3-2A-vICA). In some embodiments the nucleic acid molecule comprises a polynucleotide encoding TRIF, a polynucleotide encoding GSDME and a polynucleotide encoding a 2A peptide located between the polynucleotide encoding TRIF and the polynucleotide encoding GSDME (e.g., TRIF-2A-GSDME). In some embodiments the nucleic acid molecule comprises a polynucleotide encoding TRIF, a polynucleotide encoding Npro and a polynucleotide encoding a 2A peptide located between the polynucleotide encoding TRIF and the polynucleotide encoding Npro (e.g., TRIF-2A-Npro). In some embodiments the nucleic acid molecule comprises a polynucleotide encoding TRIF, a polynucleotide encoding A238L and a polynucleotide encoding a 2A peptide located between the polynucleotide encoding TRIF and the polynucleotide encoding A238L (e.g., TRIF-2A-A238L). In some embodiments the nucleic acid molecule comprises a polynucleotide encoding TRIF, a polynucleotide encoding vMLKL and a polynucleotide encoding a 2A peptide located between the polynucleotide encoding TRIF and the polynucleotide encoding vMLKL (e.g., TRIF-2A-vMLKL). In some embodiments the nucleic acid molecule comprises a polynucleotide encoding TRIF, a polynucleotide encoding a dominant negative variant of IKBa and a polynucleotide encoding a 2A peptide located between the polynucleotide encoding TRIF and the polynucleotide encoding the dominant negative variant of IKBa (e.g., TRIF-2A-IKBa_DN).

### IV. Target Cells

The IL-12 and thanotransmission polypeptides described herein may be expressed in a range of different target cells to promote thanotransmission by the target cell. Types of target cells include, but are not limited to, cancer cells, immune cells, endothelial cells, and fibroblasts.

Cells of any of the cancers described herein may be suitable as target cells for the engineered virus or the mRNAs expressing IL-12 and the thanotransmission polypeptides. In some embodiments, the target cell is a metastatic cancer cell.

In some embodiments, the target cell is an immune cell selected from mast cells, natural killer (NK) cells, monocytes, macrophages, dendritic cells, lymphocytes (e.g., B-cells and T cells) and any of the other immune cells described herein.

In some embodiments the target cell (e.g., a cancer cell) is deficient in a cell turnover pathway. For example, the target cell may have an inactivating mutation or copy number loss of a gene encoding a protein that contributes to the cell turnover pathway. In some embodiments, the target cell is deficient in an immune-stimulatory cell turnover pathway, e.g., necroptosis, extrinsic apoptosis, ferroptosis, pyroptosis or combinations thereof. In some embodiments, the target cell has an inactivating mutation of one or more of a gene encoding receptor-interacting serine/threonine-protein kinase 3 (RIPK3), a gene encoding a gasdermin (Gasdermin E), and a gene encoding Toll/interleukin-1 receptor (TIR)-domain-containing adapter-inducing interferon-0 (TRIF). In some embodiments, the target cell has reduced expression or activity of one or more of RIPK3, TRIF, and Gasdermin E. In some embodiments, the target cell has copy number loss of one or more of a gene encoding RIPK3, a gene encoding TRIF, and a gene encoding Gasdermin E.

The thanotransmission polypeptides may alter a cell turnover pathway in a target cell.

For example, the thanotransmission polypeptides may change the normal cell turnover pathway of the target cell to a cell turnover pathway that promotes thanotransmission, such as, e.g., necroptosis, extrinsic apoptosis, ferroptosis or pyroptosis.

In certain aspects, the disclosure relates to a cell comprising one or more of the recombinant nucleic acid molecules, vectors and/or thanotransmission polypeptides described herein.

In certain aspects, the disclosure relates to a cell comprising two or more exogenous polynucleotides each encoding a different polypeptide, wherein at least one of the exogenous polynucleotides encodes TRIF or a variant thereof, and at least one of the exogenous polynucleotides encodes a polypeptide selected from the group consisting of: RIPK3, Gasdermin E, vICA, Npro, A238L, vMLKL, variants thereof, and a dominant negative variant of IKBa. In some embodiments, at least one of the exogenous polynucleotides encodes TRIF or a variant thereof, and at least one of the exogenous polynucleotides encodes Gasdermin E or a variant thereof.

In certain aspects, the disclosure relates to a cell comprising two or more exogenous polynucleotides each encoding a different polypeptide, wherein one or more of the exogenous polynucleotides encodes IL-12 or a variant thereof, and at least one of the exogenous polynucleotides encodes a polypeptide selected from the group consisting of: TRIF, RIPK3, Gasdermin E, vICA, Npro, A238L, vMLKL, variants thereof, and a dominant negative variant of IKBa. In some embodiments, one or more of the exogenous polynucleotides encodes IL-12 or a variant thereof, and at least one of the exogenous polynucleotides encodes TRIF or a variant thereof. In some embodiments, one or more of the exogenous polynucleotides encodes IL-12 or a variant thereof, and at least one of the exogenous polynucleotides encodes Gasdermin E or a variant thereof. In some embodiments, one or more of the exogenous polynucleotides encodes IL-12 or a variant thereof, at least one of the exogenous polynucleotides encodes TRIF or a variant thereof, and at least one of the exogenous polynucleotides encodes Gasdermin E or a variant thereof. In some embodiments, the IL-12 or variant thereof is encoded by two separate polynucleotides, one encoding the p35 subunit of IL-12, and one encoding the p40 subunit of IL-12.

### V. Modes of Administering Recombinant Nucleic Acid Molecules

In certain aspects, the disclosure relates to a method of delivering one or more recombinant nucleic acid molecules to a subject, the method comprising administering a pharmaceutical composition comprising: a) one or more recombinant nucleic acid molecules encoding IL-12 and/or one or more thanotransmission polypeptides as described herein, and b) a pharmaceutically acceptable carrier, to the subject. In some embodiments, the recombinant nucleic acid molecule is a DNA molecule. In some embodiments, the recombinant nucleic acid molecule is an RNA molecule. In some embodiments, the DNA molecule or RNA molecule is comprised within a virus. In some embodiments, the DNA molecule is comprised within a plasmid or transposon. Accordingly, the one or more recombinant nucleic acid molecules encoding IL-12 and/or one or more thanotransmission polypeptides as described herein may be delivered to a subject by various modes of administration, including but not limited to as DNA molecules, as RNA molecules, or as viruses (e.g., DNA viruses or retroviruses) engineered to comprise the one or more recombinant nucleic acid molecules. In some embodiments, the one or more recombinant nucleic acid molecules is delivered to the subject through lipofection. Lipofection, also known as “lipid transfection” or “liposome-based transfection,” uses a lipid complex (e.g., a liposome) to deliver nucleic acid molecules (e.g., DNA or RNA) to cells. In some embodiments, the lipofection is RNA lipofection. In some embodiments, the lipofection is DNA lipofection.

### A. DNA Delivery Methods

In some embodiments, the recombinant nucleic acid molecules as described herein are delivered to a subject as DNA. In some embodiments, the recombinant DNA molecules are not comprised within a virus, bacterium, or other organism.

For example, in some embodiments, the recombinant nucleic acid molecule is comprised within a DNA plasmid. In some embodiments, the recombinant DNA molecules encoding the thanotransmission polypeptides are comprised within a transposon.

The polynucleotides encoding the thanotransmission polypeptides may each be operably linked to a promoter. In some embodiments, the promoter is a polymerase II (Pol II) promoter. Suitable Pol II promoters include but are not limited to a cytomegalovirus (CMV) promoter or an SV40 promoter (e.g. pcDNA3.1, pVAX1, pVIVO2, pCI, pCMV and pSV2). In a particular embodiment, the promoter is a cytomegalovirus (CMV) promoter, an EF1a promoter, or a UBC1 promoter. In some embodiments, the promoter is a tissue-specific promoter. In some embodiments, the promoter is a synthetic promoter. Suitable promoters for DNA delivery are known in the art and are described, for example, in Li, L, et al., 2016, Expert Rev Vaccines 15:313-29, which is incorporated by reference herein in its entirety. In some embodiments, the promoter is selected from the group consisting of a CMV promoter (e.g., a mini-CMV promoter), an EF1α promoter (e.g., a mini-EF1α promoter), an SV40 promoter, a PGK1 promoter, a polyubiquitin C (UBC) gene promoter, a human beta actin promoter, and a CMV enhancer/chicken beta-actin/rabbit beta-globin (CAG) hybrid promoter. In some embodiments, the promoter is a cancer-specific promoter, e.g., a tumor-specific promoter. Suitable tumor-specific promoters include, but are not limited to, a human telomerase reverse transcriptase (hTERT) promoter and an E2F promoter. The hTERT promoter drives gene expression in cells (such as cancer cells) with increased expression of telomerase. The E2F promoter drives gene expression that is specific to cells with an altered Rb pathway.

The recombinant DNA molecules encoding the thanotransmission polypeptides may each be operably linked to a DNA sequence encoding a 3′ polyadenylation (poly A) signal. In some embodiments, the poly A signal is a rabbit β-globin poly A signal or a bovine growth hormone poly A signal. The poly A signal is involved in nuclear export, translation and stability of the transcript mRNA. See Williams, J A, et al. 2013, Vaccines 1:225-49.

Methods of formulating the DNA for delivery to a subject include, but are not limited to, encapsulation in lipid nanoparticles containing cationic lipids and cholesterol, adsorption to polymers such as polyethyleneimine, and adsorption or encapsulation in biodegradable nanoparticles, such as poly(lactic-co-glycolic acid) (PLGA) or chitosan. See Donnelly J J, et al., 2005, J Immunol. 175:633-9.

The sequences of the recombinant nucleic acid molecules encoding the thanotransmission polypeptides may be codon optimized, e.g., by using enrichment of the GC content (see Thess A, et al., 2015, Mol Ther. 23:1456-64; Petsch B et al., 2012, Nat Biotechnol. 30:1210-6; and Kudla G et al., 2006, PLoS Biol. 4:e180. doi: 10.1371/journal.pbio.0040180) and/or by replacement of rare codons. Codon optimization, in some embodiments, may be used to match codon frequencies in target and host organisms to ensure proper folding; bias GC content to increase mRNA stability or reduce secondary structures; minimize tandem repeat codons or base runs that may impair gene construction or expression; customize transcriptional and translational control regions; insert or remove protein trafficking sequences; remove/add post translation modification sites in encoded protein (e.g. glycosylation sites); add, remove or shuffle protein domains; insert or delete restriction sites; modify ribosome binding sites and mRNA degradation sites; adjust translational rates to allow the various domains of the protein to fold properly; or to reduce or eliminate problem secondary structures within the polynucleotide. Codon optimization tools, algorithms and services are known in the art—non-limiting examples include services from GeneArt (Life Technologies), DNA2.0 (Menlo Park Calif) and/or proprietary methods. In some embodiments, the open reading frame (ORF) sequence is optimized using optimization algorithms.

In some embodiments a codon-optimized DNA may, for instance, be one in which the levels of G/C are enhanced. The G/C-content of nucleic acid molecules may influence the stability of the corresponding RNA. RNA having an increased amount of guanine (G) and/or cytosine (C) residues may be functionally more stable than nucleic acids containing a large amount of adenine (A) and thymine (T) or uracil (U) nucleotides. WO02/098443 discloses a pharmaceutical composition containing an mRNA stabilized by sequence modifications in the translated region. Due to the degeneracy of the genetic code, the modifications work by substituting existing codons for those that promote greater RNA stability without changing the resulting amino acid. The approach is limited to coding regions of the DNA/RNA.

The recombinant DNA molecules encoding the thanotransmission polypeptides may be delivered to a subject with synthetic delivery vehicles, such as lipid nanoparticles (LNPs). Lipid nanoparticles suitable for DNA molecule delivery are known in the art and are described, for example, in Reichmuth A M, et al., 2016, Ther Deliv. 7(5):319-334; Geall A J, et al., 2012, Proc Natl Acad Sci USA. 109:14604-9; and U.S. Pat. No. 10,702,600, each of which is incorporated by reference herein in its entirety. Suitable lipids and lipid complexes for use in lipid nanoparticles include, but are not limited to, DLinDMA: 1,2-dilinoleyloxy-3-dimethylaminopropane; DOPE: 1,2-dioleoyl-sn-glycero-3-phosphoethanolamine; DOTAP: 1,2-Dioleyl-3-trimethylammonium-propane chloride salt; DSPC: 1,2-Diastearoyl-sn-glycero-3-phosphocholine; Histidylated lipoplex: PEGylated derivative of histidylated polylysine and L-histidine-(N,N-di-n-hexadecylamine)ethylamide liposomes; HVJ-liposome: liposome with fusion proteins derived from the hemagglutinating virus of Japan (HVJ); Man11-LPR100: Mannosylated and histidylated lipopolyplexes (Man11-LPR100) obtained by adding mannosylated and histidylated liposomes to mRNA-PEGylated histidylated polylysine polyplexes; PC: Dipalmitoylphosphatidylcholine; cholesterol, PEG DMG 2000: 1,2-dimyristoyl-sn-glycero-3-phosphoethanolamine-N-[methoxy(polyethylene glycol)-2000]; PS: Phosphatidylserine; Span 85: sorbitane trioleate; unifectin; and squalene. See Martinon F, et al., 1993, Eur. J. Immunol. 23(7), 1719-1722; Hess P R, et al., 2005, Cancer Immunol. Immunother. 55(6), 672-683. Zhou W-Z, et al., 1999. Hum. Gene Ther. 10(16), 2719-2724; Pollard C, et al., 2013, Mol. Ther. 21(1), 251-259; Hoerr I, et al., 2000, Eur. J. Immunol. 30(1), 1-7; Mockey M, et al., 2007, Cancer Gene Ther. 14(9), 802-814; Perche F, et al., 2011, RNA. Nanomed. Nanotechnol. Biol. Med. 7(4), 445-453; Phua KKL, et al., 2014, Sci. Rep. 4, 5128; Geall A J, et al., 2012, Proc. Natl Acad. Sci. USA 109(36), 14604-14609; and Brito L A, et al., 2014, Mol. Ther. 22(12), 2118-2129.

In some embodiments, the lipid nanoparticle comprises a cationic lipid, a PEG-modified lipid, a sterol and a non-cationic lipid. In some embodiments, a cationic lipid is an ionizable cationic lipid and the non-cationic lipid is a neutral lipid, and the sterol is a cholesterol. In some embodiments, a cationic lipid is selected from the group consisting of 2,2-dilinoleyl-4-dimethylaminoethyl-[1,3]-dioxolane (DLin-KC2-DMA), dilinoleyl-methyl-4-dimethylaminobutyrate (DLin-MC3-DMA), di((Z)-non-2-en-1-yl) 9-((4-(dimethylamino)butanoyl)oxy)heptadecanedioate (L319), (12Z,15Z)—N,N-dimethyl-2-nonylhenicosa-12,15-dien-1-amine (L608), and N,N-dimethyl-1-[(1S,2R)-2-octylcyclopropyl]heptadecan-8-amine (L530). In some embodiments, the lipid is (L608).

The DNA molecules may also be formulated using liposomes. Liposomes are artificially prepared vesicles which may primarily be composed of a lipid bilayer and may be used as a delivery vehicle for the administration of nutrients and pharmaceutical formulations. Liposomes can be of different sizes such as, but not limited to, a multilamellar vesicle (MLV) which may be hundreds of nanometers in diameter and may contain a series of concentric bilayers separated by narrow aqueous compartments, a small unicellular vesicle (SUV) which may be smaller than 50 nm in diameter, and a large unilamellar vesicle (LUV) which may be between 50 and 500 nm in diameter. Liposome design may include, but is not limited to, opsonins or ligands in order to improve the attachment of liposomes to unhealthy tissue or to activate events such as, but not limited to, endocytosis. Liposomes may contain a low or a high pH in order to improve the delivery of the pharmaceutical formulations.

The formation of liposomes may depend on the physicochemical characteristics such as, but not limited to, the pharmaceutical formulation entrapped and the liposomal ingredients, the nature of the medium in which the lipid vesicles are dispersed, the effective concentration of the entrapped substance and its potential toxicity, any additional processes involved during the application and/or delivery of the vesicles, the optimization size, polydispersity and the shelf-life of the vesicles for the intended application, and the batch-to-batch reproducibility and possibility of large-scale production of safe and efficient liposomal products.

In some embodiments, pharmaceutical compositions described herein may include, without limitation, liposomes such as those formed from 1,2-dioleyloxy-N,N-dimethylaminopropane (DODMA) liposomes, DiLa2 liposomes from Marina Biotech (Bothell, Wash.), 1,2-dilinoleyloxy-3-dimethylaminopropane (DLin-DMA), 2,2-dilinoleyl-4-(2-dimethylaminoethyl)-[1,3]-dioxolane (DLin-KC2-DMA), and MC3 (US20100324120; herein incorporated by reference in its entirety) and liposomes which may deliver small molecule drugs such as, but not limited to, DOXIL® from Janssen Biotech, Inc. (Horsham, Pa.).

In some embodiments, pharmaceutical compositions described herein may include, without limitation, liposomes such as those formed from the synthesis of stabilized plasmid-lipid particles (SPLP) or stabilized nucleic acid lipid particle (SNALP) that have been previously described and shown to be suitable for oligonucleotide delivery in vitro and in vivo (see Wheeler et al. Gene Therapy. 1999 6:271-281; Zhang et al. Gene Therapy. 1999 6:1438-1447; Jeffs et al. Pharm Res. 2005 22:362-372; Morrissey et al., Nat Biotechnol. 2005 2:1002-1007; Zimmermann et al., Nature. 2006 441:111-114; Heyes et al. J Contr Rel. 2005 107:276-287; Semple et al. Nature Biotech. 2010 28:172-176; Judge et al. J Clin Invest. 2009 119:661-673; deFougerolles Hum Gene Ther. 2008 19:125-132; U.S. Patent Publication No US20130122104; all of which are incorporated herein in their entireties).

In some embodiments, the DNA molecules may be formulated in a lipid vesicle, which may have crosslinks between functionalized lipid bilayers. In some embodiments, the DNA molecules may be formulated in a lipid-polycation complex. The formation of the lipid-polycation complex may be accomplished by methods known in the art and/or as described in U.S. Pub. No. 20120178702, herein incorporated by reference in its entirety. As a non-limiting example, the polycation may include a cationic peptide or a polypeptide such as, but not limited to, polylysine, polyornithine and/or polyarginine. In some embodiments, the DNA molecules may be formulated in a lipid-polycation complex, which may further include a non-cationic lipid such as, but not limited to, cholesterol or dioleoyl phosphatidylethanolamine (DOPE).

In other embodiments, the recombinant DNA molecules encoding the thanotransmission polypeptides may be packaged and delivered in virus-like replicon particles (VRPs) produced by a helper cell line that provides the capsid and glycoprotein genes in trans. In some embodiments, the DNA molecules are delivered to a subject as free DNA, i.e. they are not complexed to another molecule. In some embodiments, the DNA molecules are delivered to a subject as protamine-complexed DNA. Protamine is a natural cationic nuclear protein expressed in testis. It is a highly specialized molecule that replaces histones during the final condensation of DNA in sperm and is known to stabilize nucleic acids. It has an arginine-rich sequence and spontaneously associates with nucleic acids in vitro. Protamine-complexed DNA provides both strong gene expression and immunostimulation. See Scheel B et al., 2005, Eur J Immunol. 35:1557-66; Fotin-Mleczek M, 2011, J Immunother. 34:1-15; Fotin-Mleczek M, et al., 2012, J Gene Med. 14:428-39; and Kowalczyk A, et al., 2016, Vaccine 34:3882-93.

Recombinant DNA Molecule Constructs

In addition to the polynucleotides encoding IL-12 and the thanotransmission polypeptides described herein, the recombinant DNA molecules may further comprise additional polynucleotides including, but not limited to, one or more polynucleotides encoding a 5′ UTR, one or more polynucleotides encoding a 2A peptide (e.g., a P2A peptide), one or more polynucleotides encoding a 3′ UTR, one or more polynucleotides encoding an miRNA binding site, and one or more polynucleotides encoding a poly A tail. In some embodiments, the one or more polynucleotides encoding a 5′ UTR, one or more polynucleotides encoding a 2A peptide (e.g., a P2A peptide), one or more polynucleotides encoding a 3′ UTR, one or more polynucleotides encoding an miRNA binding site, and one or more polynucleotides encoding a poly A tail are each operably linked to a polynucleotide encoding IL-12 or a thanotransmission polypeptide as described herein (e.g., TRIF, RIPK3, Gasdermin E, vICA, Npro, A238L, vMLKL, caspase 1, and variants thereof, or a dominant negative variant of IKBa).

In some embodiments, the recombinant DNA molecule comprises one or more polynucleotides encoding a 5′ UTR and one or more polynucleotides encoding a 3′ UTR. In some embodiments, the recombinant DNA molecule comprises one or more polynucleotides encoding a 5′ UTR, one or more polynucleotides encoding a 3′ UTR, and one or more polynucleotides encoding a poly A tail. In some embodiments, the recombinant DNA molecule comprises one or more polynucleotides encoding a 5′ UTR, one or more polynucleotides encoding a 3′ UTR, one or more polynucleotides encoding a poly A tail, and one or more polynucleotides encoding a 2A peptide (e.g., a P2A peptide). In some embodiments, the recombinant DNA molecule comprises one or more polynucleotides encoding a 5′ UTR, one or more polynucleotides encoding a 3′ UTR, one or more polynucleotides encoding a poly A tail, and one or more polynucleotides encoding an miRNA binding site. In some embodiments, the recombinant DNA molecule comprises one or more polynucleotides encoding a 5′ UTR, one or more polynucleotides encoding a 3′ UTR, one or more polynucleotides encoding a poly A tail, one or more polynucleotides encoding a 2A peptide (e.g., a P2A peptide), and one or more polynucleotides encoding an miRNA binding site.

In some embodiments, the recombinant DNA molecule comprises, from 5′ to 3′, a polynucleotide encoding a 5′ UTR (e.g., the 5′ UTR of SEQ ID NO: 33), a polynucleotide encoding IL-12 or a thanotransmission polypeptide as described herein (e.g., TRIF, RIPK3, Gasdermin E, vICA, Npro, A238L, vMLKL, caspase 1, or variants thereof, or a dominant negative variant of IKBa), a polynucleotide encoding a 3′ UTR (e.g., the 3′ UTR of SEQ ID NO: 34 or SEQ ID NO: 35), and a polynucleotide encoding a poly A tail. In some embodiments, the recombinant DNA molecule further comprises one or more polynucleotides encoding an miRNA binding site (e.g., an miR-122 binding site and/or an miR-142 binding site). In some embodiments, the one or more polynucleotides encoding an miRNA binding site are comprised within the 3′ UTR. In some embodiments, the one or more polynucleotides encoding an miRNA binding site are comprised within the 5′ UTR.

In some embodiments, the recombinant DNA molecule encodes only IL-12 or only one thanotransmission polypeptide.

For example, in some embodiments, the recombinant DNA molecule comprises, from 5′ to 3′, a polynucleotide encoding a 5′ UTR (e.g., the 5′ UTR of SEQ ID NO: 33), a polynucleotide encoding a caspase 1 variant (e.g., SEQ ID NO: 55), and a 3′ UTR (e.g., the 3′ UTR of SEQ ID NO: 34 or SEQ ID NO: 35). In some embodiments, the caspase 1 variant comprises a self-dimerization domain, e.g., a C-terminal self-dimerization domain. In some embodiments, the 3′ UTR comprises one or more polynucleotides encoding an miRNA binding site (e.g., an miR-122 binding site and/or an miR-142 binding site).

In some embodiments, the recombinant DNA molecule comprises, from 5′ to 3′, a polynucleotide encoding a 5′ UTR (e.g., the 5′ UTR of SEQ ID NO: 33), a polynucleotide encoding a TRIF variant (e.g., SEQ ID NO: 13), and a 3′ UTR (e.g., the 3′ UTR of SEQ ID NO: 34 or SEQ ID NO: 35). In some embodiments, the TRIF variant comprises an N-terminal deletion. In some embodiments, the TRIF variant comprises an N-terminal deletion of 180 amino acid residues relative to the human wildtype TRIF amino acid sequence of SEQ ID NO: 2. In some embodiments, the 3′ UTR comprises one or more polynucleotides encoding an miRNA binding site (e.g., an miR-122 binding site and/or an miR-142 binding site).

In some embodiments, the recombinant DNA molecule encodes two or more thanotransmission polypeptides.

For example, in some embodiments, the recombinant DNA molecule comprises, from 5′ to 3′, a polynucleotide encoding a 5′ UTR (e.g., the 5′ UTR of SEQ ID NO: 33), a polynucleotide encoding TRIF or a variant thereof (e.g., SEQ ID NO: 1), a polynucleotide encoding a 2A peptide (e.g., a P2A peptide (SEQ ID NO: 27)), a polynucleotide encoding Npro or a variant thereof (e.g., SEQ ID NO: 45), and a polynucleotide encoding a 3′ UTR (e.g., the 3′ UTR of SEQ ID NO: 34 or SEQ ID NO: 35). In some embodiments, the 3′ UTR comprises one or more polynucleotides encoding an miRNA binding site (e.g., an miR-122 binding site and/or an miR-142 binding site).

In some embodiments, the recombinant DNA molecule comprises, from 5′ to 3′, a polynucleotide encoding a 5′ UTR (e.g., the 5′ UTR of SEQ ID NO: 33), a polynucleotide encoding TRIF or a variant thereof (e.g., SEQ ID NO: 1), a polynucleotide encoding a 2A peptide (e.g., a P2A peptide (SEQ ID NO: 27)), a polynucleotide encoding A238L or a variant thereof (e.g., SEQ ID NO: 46), and a polynucleotide encoding a 3′ UTR (e.g., the 3′ UTR of SEQ ID NO: 34 or SEQ ID NO: 35). In some embodiments, the 3′ UTR comprises one or more polynucleotides encoding an miRNA binding site (e.g., an miR-122 binding site and/or an miR-142 binding site).

In some embodiments, the recombinant DNA molecule comprises, from 5′ to 3′, a polynucleotide encoding a 5′ UTR (e.g., the 5′ UTR of SEQ ID NO: 33), a polynucleotide encoding TRIF or a variant thereof (e.g., SEQ ID NO: 1), a polynucleotide encoding a 2A peptide (e.g., a P2A peptide (SEQ ID NO: 27)), a polynucleotide encoding GSDME or a variant thereof (e.g., SEQ ID NO: 44), and a polynucleotide encoding a 3′ UTR (e.g., the 3′ UTR of SEQ ID NO: 34 or SEQ ID NO: 35). In some embodiments, the 3′ UTR comprises one or more polynucleotides encoding an miRNA binding site (e.g., an miR-122 binding site and/or an miR-142 binding site).

In some embodiments, the recombinant DNA molecule comprises, from 5′ to 3′, a polynucleotide encoding a 5′ UTR (e.g., the 5′ UTR of SEQ ID NO: 33), a polynucleotide encoding TRIF or a variant thereof (e.g., SEQ ID NO: 1), a polynucleotide encoding a 2A peptide (e.g., a P2A peptide (SEQ ID NO: 27)), a polynucleotide encoding vMLKL or a variant thereof (e.g., SEQ ID NO: 47), and a polynucleotide encoding a 3′ UTR (e.g., the 3′ UTR of SEQ ID NO: 34 or SEQ ID NO: 35). In some embodiments, the 3′ UTR comprises one or more polynucleotides encoding an miRNA binding site (e.g., an miR-122 binding site and/or an miR-142 binding site).

In some embodiments, the recombinant DNA molecule comprises, from 5′ to 3′, a polynucleotide encoding a 5′ UTR (e.g., the 5′ UTR of SEQ ID NO: 33), a polynucleotide encoding TRIF or a variant thereof (e.g., SEQ ID NO: 1), a polynucleotide encoding a 2A peptide (e.g., a P2A peptide (SEQ ID NO: 27)), a polynucleotide encoding a dominant negative variant of IKBa (e.g., SEQ ID NO: 49), and a polynucleotide encoding a 3′ UTR (e.g., the 3′ UTR of SEQ ID NO: 34 or SEQ ID NO: 35). In some embodiments, the 3′ UTR comprises one or more polynucleotides encoding an miRNA binding site (e.g., an miR-122 binding site and/or an miR-142 binding site).

In some embodiments, the recombinant DNA molecule comprises, from 5′ to 3′, a polynucleotide encoding a 5′ UTR (e.g., the 5′ UTR of SEQ ID NO: 33), a polynucleotide encoding IL-12 or a variant thereof (e.g., a polynucleotide comprising SEQ ID NO: 61 and/or SEQ ID NO: 63), a polynucleotide encoding a 2A peptide (e.g., a P2A peptide (SEQ ID NO: 27)), a polynucleotide encoding TRIF or a variant thereof (e.g., SEQ ID NO: 1), and a polynucleotide encoding a 3′ UTR (e.g., the 3′ UTR of SEQ ID NO: 34 or SEQ ID NO: 35). In some embodiments, the 3′ UTR comprises one or more polynucleotides encoding an miRNA binding site (e.g., an miR-122 binding site and/or an miR-142 binding site).

In some embodiments, the recombinant DNA molecule comprises, from 5′ to 3′, a polynucleotide encoding a 5′ UTR (e.g., the 5′ UTR of SEQ ID NO: 33), a polynucleotide encoding IL-12 or a variant thereof (e.g., a polynucleotide comprising SEQ ID NO: 61 and/or SEQ ID NO: 63), a polynucleotide encoding a 2A peptide (e.g., a P2A peptide (SEQ ID NO: 27)), a polynucleotide encoding GSDME or a variant thereof (e.g., SEQ ID NO: 44), and a polynucleotide encoding a 3′ UTR (e.g., the 3′ UTR of SEQ ID NO: 34 or SEQ ID NO: 35). In some embodiments, the 3′ UTR comprises one or more polynucleotides encoding an miRNA binding site (e.g., an miR-122 binding site and/or an miR-142 binding site).

In some embodiments, the recombinant DNA molecule encodes three or more thanotransmission polypeptides.

For example, in some embodiments, the recombinant DNA molecule comprises, from 5′ to 3′, a polynucleotide encoding a 5′ UTR (e.g., the 5′ UTR of SEQ ID NO: 33), a polynucleotide encoding TRIF or a variant thereof (e.g., SEQ ID NO: 1), a first polynucleotide encoding a 2A peptide (e.g., a P2A peptide (SEQ ID NO: 27)), a polynucleotide encoding RIPK3 or a variant thereof (e.g., SEQ ID NO: 31), a second polynucleotide encoding a 2A peptide (e.g., a P2A peptide (SEQ ID NO: 27)), a polynucleotide encoding vICA or a variant thereof (e.g., SEQ ID NO: 48), and a polynucleotide encoding a 3′ UTR (e.g., the 3′ UTR of SEQ ID NO: 34 or SEQ ID NO: 35). In some embodiments, the 3′ UTR comprises one or more polynucleotides encoding an miRNA binding site (e.g., an miR-122 binding site and/or an miR-142 binding site).

In some embodiments, the recombinant DNA molecule comprises, from 5′ to 3′, a polynucleotide encoding a 5′ UTR (e.g., the 5′ UTR of SEQ ID NO: 33), a polynucleotide encoding IL-12 or a variant thereof (e.g., a polynucleotide comprising SEQ ID NO: 61 and/or SEQ ID NO: 63), a polynucleotide encoding a 2A peptide (e.g., a P2A peptide (SEQ ID NO: 27)), a polynucleotide encoding TRIF or a variant thereof (e.g., SEQ ID NO: 1), a polynucleotide encoding a 2A peptide (e.g., a P2A peptide (SEQ ID NO: 27)), a polynucleotide encoding GSDME or a variant thereof (e.g., SEQ ID NO: 44), and a polynucleotide encoding a 3′ UTR (e.g., the 3′ UTR of SEQ ID NO: 34 or SEQ ID NO: 35). In some embodiments, the 3′ UTR comprises one or more polynucleotides encoding an miRNA binding site (e.g., an miR-122 binding site and/or an miR-142 binding site).

In some embodiments, the recombinant DNA molecule comprises, from 5′ to 3′, a polynucleotide encoding a 5′ UTR (e.g., the 5′ UTR of SEQ ID NO: 33), a polynucleotide encoding IL-12 or a variant thereof (e.g., a polynucleotide comprising SEQ ID NO: 61 and/or SEQ ID NO: 63), a polynucleotide encoding a 2A peptide (e.g., a P2A peptide (SEQ ID NO: 27)), a polynucleotide encoding GSDME or a variant thereof (e.g., SEQ ID NO: 44), a polynucleotide encoding a 2A peptide (e.g., a P2A peptide (SEQ ID NO: 27)), a polynucleotide encoding TRIF or a variant thereof (e.g., SEQ ID NO: 1), and a polynucleotide encoding a 3′ UTR (e.g., the 3′ UTR of SEQ ID NO: 34 or SEQ ID NO: 35). In some embodiments, the 3′ UTR comprises one or more polynucleotides encoding an miRNA binding site (e.g., an miR-122 binding site and/or an miR-142 binding site).

In some embodiments, the recombinant nucleic acid molecule is a recombinant RNA molecule (e.g., a recombinant mRNA molecule) encoded by a recombinant DNA molecule described herein.

### B. RNA Delivery Methods

In some embodiments, the recombinant nucleic acid molecules encoding IL-2 and/or the one or more thanotransmission polypeptides as described herein are delivered to a subject as RNA. In some embodiments, the RNA is not comprised within a virus, bacterium, or other organism. In some embodiments, the RNA is purified, e.g., HPLC-purified. In some embodiments, the RNA is a circular RNA.

In some embodiments, the RNA is mRNA. The mRNAs encoding the thanotransmission polypeptides may be operably linked to 5′ and/or 3′ untranslated regions (UTRs). The UTRs, which can be of eukaryotic or viral origin, increase the half-life, and stability of the mRNA, resulting in higher expression of the encoded thanotransmission polypeptide (see Ross J, et al., 1985, Blood 66:1149-54; Gallie D R, et al., 1995, Gene 165:233-8; Kariko K, et al., 2012 Mol Ther. 20: 948-53; and Vivinus S, et al. 2001, Eur J Biochem. 268:1908-17).

A cap structure may be operably linked to the 5′ end of the mRNA. The cap structure is an N7-methylated guanosine linked to the first nucleotide of the mRNA via a reverse 5′ to 5′ triphosphate linkage. In addition to its role in cap-dependent initiation of protein synthesis, the mRNA cap also functions as a protective group from 5′ to 3′ exonuclease cleavage and a unique identifier for recruiting protein factors for pre-mRNA splicing, polyadenylation and nuclear export. See Ramanathan A, et al., 2016, Nucleic Acids Res. 44(16): 7511-7526. The 5′ cap structure is important for the creation of stable mature mRNA, and increases protein translation via binding to eukaryotic translation initiation factor 4E. See Gallie, D R., 1991, Genes Dev. 5:2108-16. The 5′ cap may be added either during transcription by inclusion of a cap analog or antireverse cap (ARCA) in the reaction (see Stepinski J, et al., 2001, RNA 7:1486-95) or subsequently, using the Vaccinia virus capping complex (see Venkatesan S, et al. 1980, J Biol Chem. 255, 903-908). In some embodiments, the 5′ terminal cap is 7mG(5′)ppp(5′)NlmpNp.

A poly(A) tail may be operably linked to the 3′ end of the mRNA. The poly A tail is an important regulatory element to enhance translation and can be either be encoded by the DNA template or alternatively added enzymatically post transcription (Gallie, D R., 1991, Genes Dev. 5:2108-16).

The sequence of an mRNA encoding a thanotransmission polypeptide may be codon optimized, e.g., by using either enrichment of the GC content (see Thess A, et al., 2015, Mol Ther. 23:1456-64; Petsch B et al., 2012, Nat Biotechnol. 30:1210-6; and Kudla G et al., 2006, PLoS Biol. 4:e180. doi: 10.1371/journal.pbio.0040180) or by replacement of rare codons. Codon optimization, in some embodiments, may be used to match codon frequencies in target and host organisms to ensure proper folding; bias GC content to increase mRNA stability or reduce secondary structures; minimize tandem repeat codons or base runs that may impair gene construction or expression; customize transcriptional and translational control regions; insert or remove protein trafficking sequences; remove/add post translation modification sites in encoded protein (e.g. glycosylation sites); add, remove or shuffle protein domains; insert or delete restriction sites; modify ribosome binding sites and mRNA degradation sites; adjust translational rates to allow the various domains of the protein to fold properly; or to reduce or eliminate problem secondary structures within the polynucleotide. Codon optimization tools, algorithms and services are known in the art—non-limiting examples include services from GeneArt (Life Technologies), DNA2.0 (Menlo Park Calif) and/or proprietary methods. In some embodiments, the open reading frame (ORF) sequence is optimized using optimization algorithms.

In some embodiments a codon-optimized RNA (e.g., mRNA) may, for instance, be one in which the levels of G/C are enhanced. The G/C-content of nucleic acid molecules may influence the stability of the RNA. RNA having an increased amount of guanine (G) and/or cytosine (C) residues may be functionally more stable than nucleic acids containing a large amount of adenine (A) and thymine (T) or uracil (U) nucleotides. WO02/098443 discloses a pharmaceutical composition containing an mRNA stabilized by sequence modifications in the translated region. Due to the degeneracy of the genetic code, the modifications work by substituting existing codons for those that promote greater RNA stability without changing the resulting amino acid. The approach is limited to coding regions of the RNA.

Chemically modified nucleosides may be added to the RNA (e.g. mRNA), for example. to decrease innate immune activation and/or increase translation of the RNA (e.g. mRNA). See Kariko K, et al., 2008, Mol Ther. 16:1833-40; and U.S. Pat. No. 10,702,600. In some embodiments, the RNA (e.g. mRNA) has an open reading frame encoding at least one polypeptide that comprises at least one chemical modification.

The terms “chemical modification” and “chemically modified” refer to modification with respect to adenosine (A), guanosine (G), uridine (U), thymidine (T) or cytidine (C) ribonucleosides or deoxyribnucleosides in at least one of their position, pattern, percent or population. Generally, these terms do not refer to the ribonucleotide modifications in naturally occurring 5′-terminal mRNA cap moieties. With respect to a polypeptide, the term “modification” refers to a modification relative to the canonical set of 20 amino acids. Polypeptides, as provided herein, are also considered “modified” if they contain amino acid substitutions, insertions or a combination of substitutions and insertions.

Polynucleotides (e.g., RNA polynucleotides, such as mRNA polynucleotides), in some embodiments, comprise various (more than one) different modifications. In some embodiments, a particular region of a polynucleotide contains one, two or more (optionally different) nucleoside or nucleotide modifications. In some embodiments, a modified RNA polynucleotide (e.g., a modified mRNA polynucleotide), introduced to a cell or organism, exhibits reduced degradation in the cell or organism, respectively, relative to an unmodified polynucleotide. In some embodiments, a modified RNA polynucleotide (e.g., a modified mRNA polynucleotide), introduced into a cell or organism, may exhibit reduced immunogenicity in the cell or organism, respectively (e.g., a reduced innate response).

Polynucleotides (e.g., RNA polynucleotides, such as mRNA polynucleotides) may comprise modifications that are naturally-occurring, non-naturally-occurring or the polynucleotide may comprise a combination of naturally-occurring and non-naturally-occurring modifications. Polynucleotides may include any useful modification, for example, of a sugar, a nucleobase, or an internucleoside linkage (e.g., to a linking phosphate, to a phosphodiester linkage or to the phosphodiester backbone).

Polynucleotides (e.g., RNA polynucleotides, such as mRNA polynucleotides), in some embodiments, comprise non-natural modified nucleotides that are introduced during synthesis or post-synthesis of the polynucleotides to achieve desired functions or properties. The modifications may be present on an internucleotide linkages, purine or pyrimidine bases, or sugars. The modification may be introduced with chemical synthesis or with a polymerase enzyme at the terminal of a chain or anywhere else in the chain. Any of the regions of a polynucleotide may be chemically modified.

The present disclosure provides for modified nucleosides and nucleotides of a polynucleotide (e.g., RNA polynucleotides, such as mRNA polynucleotides). A “nucleoside” refers to a compound containing a sugar molecule (e.g., a pentose or ribose) or a derivative thereof in combination with an organic base (e.g., a purine or pyrimidine) or a derivative thereof (also referred to herein as “nucleobase”). A nucleotide” refers to a nucleoside, including a phosphate group. Modified nucleotides may by synthesized by any useful method, such as, for example, chemically, enzymatically, or recombinantly, to include one or more modified or non-natural nucleosides. Polynucleotides may comprise a region or regions of linked nucleosides. Such regions may have variable backbone linkages. The linkages may be standard phosphodiester linkages, in which case the polynucleotides would comprise regions of nucleotides.

Modified nucleotide base pairing encompasses not only the standard adenosine-thymine, adenosine-uracil, or guanosine-cytosine base pairs, but also base pairs formed between nucleotides and/or modified nucleotides comprising non-standard or modified bases, wherein the arrangement of hydrogen bond donors and hydrogen bond acceptors permits hydrogen bonding between a non-standard base and a standard base or between two complementary non-standard base structures. One example of such non-standard base pairing is the base pairing between the modified nucleotide inosine and adenine, cytosine or uracil. Any combination of base/sugar or linker may be incorporated into polynucleotides of the present disclosure.

Modifications of polynucleotides (e.g., RNA polynucleotides, such as mRNA polynucleotides) that are useful in the RNA molecules of the present disclosure include, but are not limited to the following: 2-methylthio-N6-(cis-hydroxyisopentenyl)adenosine; 2-methylthio-N6-methyladenosine; 2-methylthio-N6-threonyl carbamoyladenosine; N6-glycinylcarbamoyladenosine; N6-isopentenyladenosine; N6-methyladenosine; N6-threonylcarbamoyladenosine; 1,2′-O-dimethyladenosine; 1-methyladenosine; 2′-O-methyladenosine; 2′-O-ribosyladenosine (phosphate); 2-methyladenosine; 2-methylthio-N6 isopentenyladenosine; 2-methylthio-N6-hydroxynorvalyl carbamoyladenosine; 2′-O-methyladenosine; 2′-O-ribosyladenosine (phosphate); Isopentenyladenosine; N6-(cis-hydroxyisopentenyl)adenosine; N6,2′-O-dimethyladenosine; N6,2′-O-dimethyladenosine; N6,N6,2′-O-trimethyladenosine; N6,N6-dimethyladenosine; N6-acetyladenosine; N6-hydroxynorvalylcarbamoyladenosine; N6-methyl-N6-threonylcarbamoyladenosine; 2-methyladenosine; 2-methylthio-N6-isopentenyladenosine; 7-deaza-adenosine; N1-methyl-adenosine; N6, N6 (dimethyl)adenine; N6-cis-hydroxy-isopentenyl-adenosine; α-thio-adenosine; 2 (amino)adenine; 2 (aminopropyl)adenine; 2 (methylthio) N6 (isopentenyl)adenine; 2-(alkyl)adenine; 2-(aminoalkyl)adenine; 2-(aminopropyl)adenine; 2-(halo)adenine; 2-(halo)adenine; 2-(propyl)adenine; 2′-Amino-2′-deoxy-ATP; 2′-Azido-2′-deoxy-ATP; 2′-Deoxy-2′-a-aminoadenosine TP; 2′-Deoxy-2′-a-azidoadenosine TP; 6 (alkyl)adenine; 6 (methyl)adenine; 6-(alkyl)adenine; 6-(methyl)adenine; 7 (deaza)adenine; 8 (alkenyl)adenine; 8 (alkynyl)adenine; 8 (amino)adenine; 8 (thioalkyl)adenine; 8-(alkenyl)adenine; 8-(alkyl)adenine; 8-(alkynyl)adenine; 8-(amino)adenine; 8-(halo)adenine; 8-(hydroxyl)adenine; 8-(thioalkyl)adenine; 8-(thiol)adenine; 8-azido-adenosine; aza adenine; deaza adenine; N6 (methyl)adenine; N6-(isopentyl)adenine; 7-deaza-8-aza-adenosine; 7-methyladenine; 1-Deazaadenosine TP; 2′Fluoro-N6-Bz-deoxyadenosine TP; 2′-OMe-2-Amino-ATP; 2′O-methyl-N6-Bz-deoxyadenosine TP; 2′-a-Ethynyladenosine TP; 2-aminoadenine; 2-Aminoadenosine TP; 2-Amino-ATP; 2′-a-Trifluoromethyladenosine TP; 2-Azidoadenosine TP; 2′-b-Ethynyladenosine TP; 2-Bromoadenosine TP; 2′-b-Trifluoromethyladenosine TP; 2-Chloroadenosine TP; 2′-Deoxy-2′, 2′-difluoroadenosine TP; 2′-Deoxy-2′-a-mercaptoadenosine TP; 2′-Deoxy-2′-a-thiomethoxyadenosine TP; 2′-Deoxy-2′-b-aminoadenosine TP; 2′-Deoxy-2′-b-azidoadenosine TP; 2′-Deoxy-2′-b-bromoadenosine TP; 2′-Deoxy-2′-b-chloroadenosine TP; 2′-Deoxy-2′-b-fluoroadenosine TP; 2′-Deoxy-2′-b-iodoadenosine TP; 2′-Deoxy-2′-b-mercaptoadenosine TP; 2′-Deoxy-2′-b-thiomethoxyadenosine TP; 2-Fluoroadenosine TP; 2-lodoadenosine TP; 2-Mercaptoadenosine TP; 2-methoxy-adenine; 2-methylthio-adenine; 2-Trifluoromethyladenosine TP; 3-Deaza-3-bromoadenosine TP; 3-Deaza-3-chloroadenosine TP; 3-Deaza-3-fluoroadenosine TP; 3-Deaza-3-iodoadenosine TP; 3-Deazaadenosine TP; 4′-Azidoadenosine TP; 4′-Carbocyclic adenosine TP; 4′-Ethynyladenosine TP; 5′-Homo-adenosine TP; 8-Aza-ATP; 8-bromo-adenosine TP; 8-Trifluoromethyladenosine TP; 9-Deazaadenosine TP; 2-aminopurine; 7-deaza-2,6-diaminopurine; 7-deaza-8-aza-2,6-diaminopurine; 7-deaza-8-aza-2-aminopurine; 2,6-diaminopurine; 7-deaza-8-aza-adenine, 7-deaza-2-aminopurine; 2-thiocytidine; 3-methylcytidine; 5-formylcytidine; 5-hydroxymethylcytidine; 5-methylcytidine; N4-acetylcytidine; 2′-O-methylcytidine; 2′-O-methylcytidine; 5,2′-O-dimethylcytidine; 5-formyl-2′-O-methylcytidine; Lysidine; N4,2′-O-dimethylcytidine; N4-acetyl-2′-O-methylcytidine; N4-methylcytidine; N4,N4-Dimethyl-2′-OMe-Cytidine TP; 4-methylcytidine; 5-aza-cytidine; Pseudo-iso-cytidine; pyrrolo-cytidine; α-thio-cytidine; 2-(thio)cytosine; 2′-Amino-2′-deoxy-CTP; 2′-Azido-2′-deoxy-CTP; 2′-Deoxy-2′-a-aminocytidine TP; 2′-Deoxy-2′-a-azidocytidine TP; 3 (deaza) 5 (aza)cytosine; 3 (methyl)cytosine; 3-(alkyl)cytosine; 3-(deaza) 5 (aza)cytosine; 3-(methyl)cytidine; 4,2′-O-dimethylcytidine; 5 (halo)cytosine; 5 (methyl)cytosine; 5 (propynyl)cytosine; 5 (trifluoromethyl)cytosine; 5-(alkyl)cytosine; 5-(alkynyl)cytosine; 5-(halo)cytosine; 5-(propynyl)cytosine; 5-(trifluoromethyl)cytosine; 5-bromo-cytidine; 5-iodo-cytidine; 5-propynyl cytosine; 6-(azo)cytosine; 6-aza-cytidine; aza cytosine; deaza cytosine; N4 (acetyl)cytosine; 1-methyl-1-deaza-pseudoisocytidine; 1-methyl-pseudoisocytidine; 2-methoxy-5-methyl-cytidine; 2-methoxy-cytidine; 2-thio-5-methyl-cytidine; 4-methoxy-1-methyl-pseudoisocytidine; 4-methoxy-pseudoisocytidine; 4-thio-1-methyl-1-deaza-pseudoisocytidine; 4-thio-1-methyl-pseudoisocytidine; 4-thio-pseudoisocytidine; 5-aza-zebularine; 5-methyl-zebularine; pyrrolo-pseudoisocytidine; Zebularine; (E)-5-(2-Bromo-vinyl)cytidine TP; 2,2′-anhydro-cytidine TP hydrochloride; 2′Fluor-N4-Bz-cytidine TP; 2′Fluoro-N4-Acetyl-cytidine TP; 2′-O-Methyl-N4-Acetyl-cytidine TP; 2′O-methyl-N4-Bz-cytidine TP; 2′-a-Ethynylcytidine TP; 2′-a-Trifluoromethylcytidine TP; 2′-b-Ethynylcytidine TP; 2′-b-Trifluoromethylcytidine TP; 2′-Deoxy-2′, 2′-difluorocytidine TP; 2′-Deoxy-2′-a-mercaptocytidine TP; 2′-Deoxy-2′-a-thiomethoxycytidine TP; 2′-Deoxy-2′-b-aminocytidine TP; 2′-Deoxy-2′-b-azidocytidine TP; 2′-Deoxy-2′-b-bromocytidine TP; 2′-Deoxy-2′-b-chlorocytidine TP; 2′-Deoxy-2′-b-fluorocytidine TP; 2′-Deoxy-2′-b-iodocytidine TP; 2′-Deoxy-2′-b-mercaptocytidine TP; 2′-Deoxy-2′-b-thiomethoxycytidine TP; 2′-O-Methyl-5-(1-propynyl)cytidine TP; 3′-Ethynylcytidine TP; 4′-Azidocytidine TP; 4′-Carbocyclic cytidine TP; 4′-Ethynylcytidine TP; 5-(1-Propynyl)ara-cytidine TP; 5-(2-Chloro-phenyl)-2-thiocytidine TP; 5-(4-Amino-phenyl)-2-thiocytidine TP; 5-Aminoallyl-CTP; 5-Cyanocytidine TP; 5-Ethynylara-cytidine TP; 5-Ethynylcytidine TP; 5′-Homo-cytidine TP; 5-Methoxycytidine TP; 5-Trifluoromethyl-Cytidine TP; N4-Amino-cytidine TP; N4-Benzoyl-cytidine TP; Pseudoisocytidine; 7-methylguanosine; N2,2′-O-dimethylguanosine; N2-methylguanosine; Wyosine; 1,2′-O-dimethylguanosine; 1-methylguanosine; 2′-O-methylguanosine; 2′-O-ribosylguanosine (phosphate); 2′-O-methylguanosine; 2′-O-ribosylguanosine (phosphate); 7-aminomethyl-7-deazaguanosine; 7-cyano-7-deazaguanosine; Archaeosine; Methylwyosine; N2,7-dimethylguanosine; N2,N2,2′-O-trimethylguanosine; N2,N2,7-trimethylguanosine; N2,N2-dimethylguanosine; N2,7,2′-O-trimethylguanosine; 6-thio-guanosine; 7-deaza-guanosine; 8-oxo-guanosine; N1-methyl-guanosine; α-thio-guanosine; 2 (propyl)guanine; 2-(alkyl)guanine; 2′-Amino-2′-deoxy-GTP; 2′-Azido-2′-deoxy-GTP; 2′-Deoxy-2′-a-aminoguanosine TP; 2′-Deoxy-2′-a-azidoguanosine TP; 6 (methyl)guanine; 6-(alkyl)guanine; 6-(methyl)guanine; 6-methyl-guanosine; 7 (alkyl)guanine; 7 (deaza)guanine; 7 (methyl)guanine; 7-(alkyl)guanine; 7-(deaza)guanine; 7-(methyl)guanine; 8 (alkyl)guanine; 8 (alkynyl)guanine; 8 (halo)guanine; 8 (thioalkyl)guanine; 8-(alkenyl)guanine; 8-(alkyl)guanine; 8-(alkynyl)guanine; 8-(amino)guanine; 8-(halo)guanine; 8-(hydroxyl)guanine; 8-(thioalkyl)guanine; 8-(thiol)guanine; aza guanine; deaza guanine; N (methyl)guanine; N-(methyl)guanine; 1-methyl-6-thio-guanosine; 6-methoxy-guanosine; 6-thio-7-deaza-8-aza-guanosine; 6-thio-7-deaza-guanosine; 6-thio-7-methyl-guanosine; 7-deaza-8-aza-guanosine; 7-methyl-8-oxo-guanosine; N2,N2-dimethyl-6-thio-guanosine; N2-methyl-6-thio-guanosine; 1-Me-GTP; 2′Fluoro-N2-isobutyl-guanosine TP; 2′O-methyl-N2-isobutyl-guanosine TP; 2′-a-Ethynylguanosine TP; 2′-a-Trifluoromethylguanosine TP; 2′-b-Ethynylguanosine TP; 2′-b-Trifluoromethylguanosine TP; 2′-Deoxy-2′, 2′-difluoroguanosine TP; 2′-Deoxy-2′-a-mercaptoguanosine TP; 2′-Deoxy-2′-a-thiomethoxyguanosine TP; 2′-Deoxy-2′-b-aminoguanosine TP; 2′-Deoxy-2′-b-azidoguanosine TP; 2′-Deoxy-2′-b-bromoguanosine TP; 2′-Deoxy-2′-b-chloroguanosine TP; 2′-Deoxy-2′-b-fluoroguanosine TP; 2′-Deoxy-2′-b-iodoguanosine TP; 2′-Deoxy-2′-b-mercaptoguanosine TP; 2′-Deoxy-2′-b-thiomethoxyguanosine TP; 4′-Azidoguanosine TP; 4′-Carbocyclic guanosine TP; 4′-Ethynylguanosine TP; 5′-Homo-guanosine TP; 8-bromo-guanosine TP; 9-Deazaguanosine TP; N2-isobutyl-guanosine TP; 1-methylinosine; Inosine; 1,2′-O-dimethylinosine; 2′-O-methylinosine; 7-methylinosine; 2′-O-methylinosine; Epoxyqueuosine; galactosyl-queuosine; Mannosylqueuosine; Queuosine; allyamino-thymidine; aza thymidine; deaza thymidine; deoxy-thymidine; 2′-O-methyluridine; 2-thiouridine; 3-methyluridine; 5-carboxymethyluridine; 5-hydroxyuridine; 5-methyluridine; 5-taurinomethyl-2-thiouridine; 5-taurinomethyluridine; Dihydrouridine; Pseudouridine; (3-(3-amino-3-carboxypropyl)uridine; 1-methyl-3-(3-amino-5-carboxypropyl)pseudouridine; 1-methylpseduouridine; 1-methyl-pseudouridine; 2′-O-methyluridine; 2′-O-methylpseudouridine; 2′-O-methyluridine; 2-thio-2′-O-methyluridine; 3-(3-amino-3-carboxypropyl)uridine; 3,2′-O-dimethyluridine; 3-Methyl-pseudo-Uridine TP; 4-thiouridine; 5-(carboxyhydroxymethyl)uridine; 5-(carboxyhydroxymethyl)uridine methyl ester; 5,2′-O-dimethyluridine; 5,6-dihydro-uridine; 5-aminomethyl-2-thiouridine; 5-carbamoylmethyl-2′-O-methyluridine; 5-carbamoylmethyluridine; 5-carboxyhydroxymethyluridine; 5-carboxyhydroxymethyluridine methyl ester; 5-carboxymethylaminomethyl-2′-O-methyluridine; 5-carboxymethylaminomethyl-2-thiouridine; 5-carboxymethylaminomethyl-2-thiouridine; 5-carboxymethylaminomethyluridine; 5-carboxymethylaminomethyluridine; 5-Carbamoylmethyluridine TP; 5-methoxycarbonylmethyl-2′-O-methyluridine; 5-methoxycarbonylmethyl-2-thiouridine; 5-methoxycarbonylmethyluridine; 5-methoxyuridine; 5-methyl-2-thiouridine; 5-methylaminomethyl-2-selenouridine; 5-methylaminomethyl-2-thiouridine; 5-methylaminomethyluridine; 5-Methyldihydrouridine; 5-Oxyacetic acid-Uridine TP; 5-Oxyacetic acid-methyl ester-Uridine TP; N1-methyl-pseudo-uridine; uridine 5-oxyacetic acid; uridine 5-oxyacetic acid methyl ester; 3-(3-Amino-3-carboxypropyl)-Uridine TP; 5-(iso-Pentenylaminomethyl)-2-thiouridine TP; 5-(iso-Pentenylaminomethyl)-2′-O-methyluridine TP; 5-(iso-Pentenylaminomethyl)uridine TP; 5-propynyl uracil; α-thio-uridine; 1 (aminoalkylamino-carbonylethylenyl)-2(thio)-pseudouracil; 1 (aminoalkylaminocarbonylethylenyl)-2,4-(dithio)pseudouracil; 1 (aminoalkylaminocarbonylethylenyl)-4 (thio)pseudouracil; 1 (aminoalkylaminocarbonylethylenyl)-pseudouracil; 1 (aminocarbonylethylenyl)-2(thio)-pseudouracil; 1 (aminocarbonylethylenyl)-2,4-(dithio)pseudouracil; 1 (aminocarbonylethylenyl)-4 (thio)pseudouracil; 1 (aminocarbonylethylenyl)-pseudouracil; 1 substituted 2(thio)-pseudouracil; 1 substituted 2,4-(dithio)pseudouracil; 1 substituted 4 (thio)pseudouracil; 1 substituted pseudouracil; 1-(aminoalkylamino-carbonylethylenyl)-2-(thio)-pseudouracil; 1-Methyl-3-(3-amino-3-carboxypropyl) pseudouridine TP; 1-Methyl-3-(3-amino-3-carboxypropyl)pseudo-UTP; 1-Methyl-pseudo-UTP; 2 (thio)pseudouracil; 2′ deoxy uridine; 2′ fluorouridine; 2-(thio)uracil; 2,4-(dithio)psuedouracil; 2′ methyl, 2′amino, 2′ azido, 2′fluro-guanosine; 2′-Amino-2′-deoxy-UTP; 2′-Azido-2′-deoxy-UTP; 2′-Azido-deoxyuridine TP; 2′-O-methylpseudouridine; 2′ deoxy uridine; 2′ fluorouridine; 2′-Deoxy-2′-a-aminouridine TP; 2′-Deoxy-2′-a-azidouridine TP; 2-methylpseudouridine; 3 (3 amino-3 carboxypropyl)uracil; 4 (thio)pseudouracil; 4-(thio)pseudouracil; 4-(thio)uracil; 4-thiouracil; 5 (1,3-diazole-1-alkyl)uracil; 5 (2-aminopropyl)uracil; 5 (aminoalkyl)uracil; 5 (dimethylaminoalkyl)uracil; 5 (guanidiniumalkyl)uracil; 5 (methoxycarbonylmethyl)-2-(thio)uracil; 5 (methoxycarbonyl-methyl)uracil; 5 (methyl) 2 (thio)uracil; 5 (methyl) 2,4 (dithio)uracil; 5 (methyl) 4 (thio)uracil; 5 (methylaminomethyl)-2 (thio)uracil; 5 (methylaminomethyl)-2,4 (dithio)uracil; 5 (methylaminomethyl)-4 (thio)uracil; 5 (propynyl)uracil; 5 (trifluoromethyl)uracil; 5-(2-aminopropyl)uracil; 5-(alkyl)-2-(thio)pseudouracil; 5-(alkyl)-2,4 (dithio)pseudouracil; 5-(alkyl)-4 (thio)pseudouracil; 5-(alkyl)pseudouracil; 5-(alkyl)uracil; 5-(alkynyl)uracil; 5-(allylamino)uracil; 5-(cyanoalkyl)uracil; 5-(dialkylaminoalkyl)uracil; 5-(dimethylaminoalkyl)uracil; 5-(guanidiniumalkyl)uracil; 5-(halo)uracil; 5-(1,3-diazole-1-alkyl)uracil; 5-(methoxy)uracil; 5-(methoxycarbonylmethyl)-2-(thio)uracil; 5-(methoxycarbonyl-methyl)uracil; 5-(methyl) 2(thio)uracil; 5-(methyl) 2,4 (dithio)uracil; 5-(methyl) 4 (thio)uracil; 5-(methyl)-2-(thio)pseudouracil; 5-(methyl)-2,4 (dithio)pseudouracil; 5-(methyl)-4 (thio)pseudouracil; 5-(methyl)pseudouracil; 5-(methylaminomethyl)-2 (thio)uracil; 5-(methylaminomethyl)-2,4(dithio)uracil; 5-(methylaminomethyl)-4-(thio)uracil; 5-(propynyl)uracil; 5-(trifluoromethyl)uracil; 5-aminoallyl-uridine; 5-bromo-uridine; 5-iodo-uridine; 5-uracil; 6 (azo)uracil; 6-(azo)uracil; 6-aza-uridine; ally amino-uracil; aza uracil; deaza uracil; N3 (methyl)uracil; Pseudo-UTP-1-2-ethanoic acid; Pseudouracil; 4-Thio-pseudo-UTP; 1-carboxymethyl-pseudouridine; 1-methyl-1-deaza-pseudouridine; 1-propynyl-uridine; 1-taurinomethyl-1-methyl-uridine; 1-taurinomethyl-4-thio-uridine; 1-taurinomethyl-pseudouridine; 2-methoxy-4-thio-pseudouridine; 2-thio-1-methyl-1-deaza-pseudouridine; 2-thio-1-methyl-pseudouridine; 2-thio-5-aza-uridine; 2-thio-dihydropseudouridine; 2-thio-dihydrouridine; 2-thio-pseudouridine; 4-methoxy-2-thio-pseudouridine; 4-methoxy-pseudouridine; 4-thio-1-methyl-pseudouridine; 4-thio-pseudouridine; 5-aza-uridine; Dihydropseudouridine; (+) 1-(2-Hydroxypropyl)pseudouridine TP; (2R)-1-(2-Hydroxypropyl)pseudouridine TP; (2S)-1-(2-Hydroxypropyl)pseudouridine TP; (E)-5-(2-Bromo-vinyl)ara-uridine TP; (E)-5-(2-Bromo-vinyl)uridine TP; (Z)-5-(2-Bromo-vinyl)ara-uridine TP; (Z)-5-(2-Bromo-vinyl)uridine TP; 1-(2,2,2-Trifluoroethyl)-pseudo-UTP; 1-(2,2,3,3,3-Pentafluoropropyl)pseudouridine TP; 1-(2,2-Diethoxyethyl)pseudouridine TP; 1-(2,4,6-Trimethylbenzyl)pseudouridine TP; 1-(2,4,6-Trimethyl-benzyl)pseudo-UTP; 1-(2,4,6-Trimethyl-phenyl)pseudo-UTP; 1-(2-Amino-2-carboxyethyl)pseudo-UTP; 1-(2-Amino-ethyl)pseudo-UTP; 1-(2-Hydroxyethyl)pseudouridine TP; 1-(2-Methoxyethyl)pseudouridine TP; 1-(3,4-Bis-trifluoromethoxybenzyl)pseudouridine TP; 1-(3,4-Dimethoxybenzyl)pseudouridine TP; 1-(3-Amino-3-carboxypropyl)pseudo-UTP; 1-(3-Amino-propyl)pseudo-UTP; 1-(3-Cyclopropyl-prop-2-ynyl)pseudouridine TP; 1-(4-Amino-4-carboxybutyl)pseudo-UTP; 1-(4-Amino-benzyl)pseudo-UTP; 1-(4-Amino-butyl)pseudo-UTP; 1-(4-Amino-phenyl)pseudo-UTP; 1-(4-Azidobenzyl)pseudouridine TP; 1-(4-Bromobenzyl)pseudouridine TP; 1-(4-Chlorobenzyl)pseudouridine TP; 1-(4-Fluorobenzyl)pseudouridine TP; 1-(4-Iodobenzyl)pseudouridine TP; 1-(4-Methanesulfonylbenzyl)pseudouridine TP; 1-(4-Methoxybenzyl)pseudouridine TP; 1-(4-Methoxy-benzyl)pseudo-UTP; 1-(4-Methoxy-phenyl)pseudo-UTP; 1-(4-Methylbenzyl)pseudouridine TP; 1-(4-Methyl-benzyl)pseudo-UTP; 1-(4-Nitrobenzyl)pseudouridine TP; 1-(4-Nitro-benzyl)pseudo-UTP; 1(4-Nitro-phenyl)pseudo-UTP; 1-(4-Thiomethoxybenzyl)pseudouridine TP; 1-(4-Trifluoromethoxybenzyl)pseudouridine TP; 1-(4-Trifluoromethylbenzyl)pseudouridine TP; 1-(5-Amino-pentyl)pseudo-UTP; 1-(6-Amino-hexyl)pseudo-UTP; 1,6-Dimethyl-pseudo-UTP; 1-[3-(2-{2-[2-(2-Aminoethoxy)-ethoxy]-ethoxy}-ethoxy)-propionyl]pseudouridine TP; 1-{3-[2-(2-Aminoethoxy)-ethoxy]-propionyl}pseudouridine TP; 1-Acetylpseudouridine TP; 1-Alkyl-6-(1-propynyl)-pseudo-UTP; 1-Alkyl-6-(2-propynyl)-pseudo-UTP; 1-Alkyl-6-allyl-pseudo-UTP; 1-Alkyl-6-ethynyl-pseudo-UTP; 1-Alkyl-6-homoallyl-pseudo-UTP; 1-Alkyl-6-vinyl-pseudo-UTP; 1-Allylpseudouridine TP; 1-Aminomethyl-pseudo-UTP; 1-Benzoylpseudouridine TP; 1-Benzyloxymethylpseudouridine TP; 1-Benzyl-pseudo-UTP; 1-Biotinyl-PEG2-pseudouridine TP; 1-Biotinylpseudouridine TP; 1-Butyl-pseudo-UTP; 1-Cyanomethylpseudouridine TP; 1-Cyclobutylmethyl-pseudo-UTP; 1-Cyclobutyl-pseudo-UTP; 1-Cycloheptylmethyl-pseudo-UTP; 1-Cycloheptyl-pseudo-UTP; 1-Cyclohexylmethyl-pseudo-UTP; 1-Cyclohexyl-pseudo-UTP; 1-Cyclooctylmethyl-pseudo-UTP; 1-Cyclooctyl-pseudo-UTP; 1-Cyclopentylmethyl-pseudo-UTP; 1-Cyclopentyl-pseudo-UTP; 1-Cyclopropylmethyl-pseudo-UTP; 1-Cyclopropyl-pseudo-UTP; 1-Ethyl-pseudo-UTP; 1-Hexyl-pseudo-UTP; 1-Homoallylpseudouridine TP; 1-Hydroxymethylpseudouridine TP; 1-iso-propyl-pseudo-UTP; 1-Me-2-thio-pseudo-UTP; 1-Me-4-thio-pseudo-UTP; 1-Me-alpha-thio-pseudo-UTP; 1-Methanesulfonylmethylpseudouridine TP; 1-Methoxymethylpseudouridine TP; 1-Methyl-6-(2,2,2-Trifluoroethyl)pseudo-UTP; 1-Methyl-6-(4-morpholino)-pseudo-UTP; 1-Methyl-6-(4-thiomorpholino)-pseudo-UTP; 1-Methyl-6-(substituted phenyl)pseudo-UTP; 1-Methyl-6-amino-pseudo-UTP; 1-Methyl-6-azido-pseudo-UTP; 1-Methyl-6-bromo-pseudo-UTP; 1-Methyl-6-butyl-pseudo-UTP; 1-Methyl-6-chloro-pseudo-UTP; 1-Methyl-6-cyano-pseudo-UTP; 1-Methyl-6-dimethylamino-pseudo-UTP; 1-Methyl-6-ethoxy-pseudo-UTP; 1-Methyl-6-ethylcarboxylate-pseudo-UTP; 1-Methyl-6-ethyl-pseudo-UTP; 1-Methyl-6-fluoro-pseudo-UTP; 1-Methyl-6-formyl-pseudo-UTP; 1-Methyl-6-hydroxyamino-pseudo-UTP; 1-Methyl-6-hydroxy-pseudo-UTP; 1-Methyl-6-iodo-pseudo-UTP; 1-Methyl-6-iso-propyl-pseudo-UTP; 1-Methyl-6-methoxy-pseudo-UTP; 1-Methyl-6-methylamino-pseudo-UTP; 1-Methyl-6-phenyl-pseudo-UTP; 1-Methyl-6-propyl-pseudo-UTP; 1-Methyl-6-tert-butyl-pseudo-UTP; 1-Methyl-6-trifluoromethoxy-pseudo-UTP; 1-Methyl-6-trifluoromethyl-pseudo-UTP; 1-Morpholinomethylpseudouridine TP; 1-Pentyl-pseudo-UTP; 1-Phenyl-pseudo-UTP; 1-Pivaloylpseudouridine TP; 1-Propargylpseudouridine TP; 1-Propyl-pseudo-UTP; 1-propynyl-pseudouridine; 1-p-tolyl-pseudo-UTP; 1-tert-Butyl-pseudo-UTP; 1-Thiomethoxymethylpseudouridine TP; 1-Thiomorpholinomethylpseudouridine TP; 1-Trifluoroacetylpseudouridine TP; 1-Trifluoromethyl-pseudo-UTP; 1-Vinylpseudouridine TP; 2,2′-anhydro-uridine TP; 2′-bromo-deoxyuridine TP; 2′-F-5-Methyl-2′-deoxy-UTP; 2′-OMe-5-Me-UTP; 2′-OMe-pseudo-UTP; 2′-a-Ethynyluridine TP; 2′-a-Trifluoromethyluridine TP; 2′-b-Ethynyluridine TP; 2′-b-Trifluoromethyluridine TP; 2′-Deoxy-2′, 2′-difluorouridine TP; 2′-Deoxy-2′-a-mercaptouridine TP; 2′-Deoxy-2′-a-thiomethoxyuridine TP; 2′-Deoxy-2′-b-aminouridine TP; 2′-Deoxy-2′-b-azidouridine TP; 2′-Deoxy-2′-b-bromouridine TP; 2′-Deoxy-2′-b-chlorouridine TP; 2′-Deoxy-2′-b-fluorouridine TP; 2′-Deoxy-2′-b-iodouridine TP; 2′-Deoxy-2′-b-mercaptouridine TP; 2′-Deoxy-2′-b-thiomethoxyuridine TP; 2-methoxy-4-thio-uridine; 2-methoxyuridine; 2′-O-Methyl-5-(1-propynyl)uridine TP; 3-Alkyl-pseudo-UTP; 4′-Azidouridine TP; 4′-Carbocyclic uridine TP; 4′-Ethynyluridine TP; 5-(1-Propynyl)ara-uridine TP; 5-(2-Furanyl)uridine TP; 5-Cyanouridine TP; 5-Dimethylaminouridine TP; 5′-Homo-uridine TP; 5-iodo-2′-fluoro-deoxyuridine TP; 5-Phenylethynyluridine TP; 5-Trideuteromethyl-6-deuterouridine TP; 5-Trifluoromethyl-Uridine TP; 5-Vinylarauridine TP; 6-(2,2,2-Trifluoroethyl)-pseudo-UTP; 6-(4-Morpholino)-pseudo-UTP; 6-(4-Thiomorpholino)-pseudo-UTP; 6-(Substituted-Phenyl)-pseudo-UTP; 6-Amino-pseudo-UTP; 6-Azido-pseudo-UTP; 6-Bromo-pseudo-UTP; 6-Butyl-pseudo-UTP; 6-Chloro-pseudo-UTP; 6-Cyano-pseudo-UTP; 6-Dimethylamino-pseudo-UTP; 6-Ethoxy-pseudo-UTP; 6-Ethylcarboxylate-pseudo-UTP; 6-Ethyl-pseudo-UTP; 6-Fluoro-pseudo-UTP; 6-Formyl-pseudo-UTP; 6-Hydroxyamino-pseudo-UTP; 6-Hydroxy-pseudo-UTP; 6-Iodo-pseudo-UTP; 6-iso-Propyl-pseudo-UTP; 6-Methoxy-pseudo-UTP; 6-Methylamino-pseudo-UTP; 6-Methyl-pseudo-UTP; 6-Phenyl-pseudo-UTP; 6-Phenyl-pseudo-UTP; 6-Propyl-pseudo-UTP; 6-tert-Butyl-pseudo-UTP; 6-Trifluoromethoxy-pseudo-UTP; 6-Trifluoromethyl-pseudo-UTP; Alpha-thio-pseudo-UTP; Pseudouridine 1-(4-methylbenzenesulfonic acid) TP; Pseudouridine 1-(4-methylbenzoic acid) TP; Pseudouridine TP 1-[3-(2-ethoxy)]propionic acid; Pseudouridine TP 1-[3-{2-(2-[2-(2-ethoxy)-ethoxy]-ethoxy)-ethoxy}]propionic acid; Pseudouridine TP 1-[3-{2-(2-[2-{2(2-ethoxy)-ethoxy}-ethoxy]-ethoxy)-ethoxy}]propionic acid; Pseudouridine TP 1-[3-{2-(2-[2-ethoxy]-ethoxy)-ethoxy}]propionic acid; Pseudouridine TP 1-[3-{2-(2-ethoxy)-ethoxy}] propionic acid; Pseudouridine TP 1-methylphosphonic acid; Pseudouridine TP 1-methylphosphonic acid diethyl ester; Pseudo-UTP-N1-3-propionic acid; Pseudo-UTP-N1-4-butanoic acid; Pseudo-UTP-N1-5-pentanoic acid; Pseudo-UTP-N1-6-hexanoic acid; Pseudo-UTP-N1-7-heptanoic acid; Pseudo-UTP-N1-methyl-p-benzoic acid; Pseudo-UTP-N1-p-benzoic acid; Wybutosine; Hydroxywybutosine; Isowyosine; Peroxywybutosine; undermodified hydroxywybutosine; 4-demethylwyosine; 2,6-(diamino)purine; 1-(aza)-2-(thio)-3-(aza)-phenoxazin-1-yl: 1,3-(diaza)-2-(oxo)-phenthiazin-1-yl; 1,3-(diaza)-2-(oxo)-phenoxazin-1-yl; 1,3,5-(triaza)-2,6-(dioxa)-naphthalene; 2 (amino)purine; 2,4,5-(trimethyl)phenyl; 2′ methyl, 2′amino, 2′azido, 2′fluro-cytidine; 2′ methyl, 2′ amino, 2′azido, 2′fluro-adenine; 2′methyl, 2′amino, 2′ azido, 2′fluro-uridine; 2′-amino-2′-deoxyribose; 2-amino-6-Chloro-purine; 2-aza-inosinyl; 2′-azido-2′-deoxyribose; 2′fluoro-2′-deoxyribose; 2′-fluoro-modified bases; 2′-O-methyl-ribose; 2-oxo-7-aminopyridopyrimidin-3-yl; 2-oxo-pyridopyrimidine-3-yl; 2-pyridinone; 3 nitropyrrole; 3-(methyl)-7-(propynyl)isocarbostyrilyl; 3-(methyl)isocarbostyrilyl; 4-(fluoro)-6-(methyl)benzimidazole; 4-(methyl)benzimidazole; 4-(methyl)indolyl; 4,6-(dimethyl)indolyl; 5 nitroindole; 5 substituted pyrimidines; 5-(methyl)isocarbostyrilyl; 5-nitroindole; 6-(aza)pyrimidine; 6-(azo)thymine; 6-(methyl)-7-(aza)indolyl; 6-chloro-purine; 6-phenyl-pyrrolo-pyrimidin-2-on-3-yl; 7-(aminoalkylhydroxy)-1-(aza)-2-(thio)-3-(aza)-phenthiazin-1-yl; 7-(aminoalkylhydroxy)-1-(aza)-2-(thio)-3-(aza)-phenoxazin-1-yl; 7-(aminoalkylhydroxy)-1,3-(diaza)-2-(oxo)-phenoxazin-1-yl; 7-(aminoalkylhydroxy)-1,3-(diaza)-2-(oxo)-phenthiazin-1-yl; 7-(aminoalkylhydroxy)-1,3-(diaza)-2-(oxo)-phenoxazin-1-yl; 7-(aza)indolyl; 7-(guanidiniumalkylhydroxy)-1-(aza)-2-(thio)-3-(aza)-phenoxazinl-yl; 7-(guanidiniumalkylhydroxy)-1-(aza)-2-(thio)-3-(aza)-phenthiazin-1-yl; 7-(guanidiniumalkylhydroxy)-1-(aza)-2-(thio)-3-(aza)-phenoxazin-1-yl; 7-(guanidiniumalkylhydroxy)-1,3-(diaza)-2-(oxo)-phenoxazin-1-yl; 7-(guanidiniumalkyl-hydroxy)-1,3-(diaza)-2-(oxo)-phenthiazin-1-yl; 7-(guanidiniumalkylhydroxy)-1,3-(diaza)-2-(oxo)-phenoxazin-1-yl; 7-(propynyl)isocarbostyrilyl; 7-(propynyl)isocarbostyrilyl, propynyl-7-(aza)indolyl; 7-deaza-inosinyl; 7-substituted 1-(aza)-2-(thio)-3-(aza)-phenoxazin-1-yl; 7-substituted 1,3-(diaza)-2-(oxo)-phenoxazin-1-yl; 9-(methyl)-imidizopyridinyl; Aminoindolyl; Anthracenyl; bis-ortho-(aminoalkylhydroxy)-6-phenyl-pyrrolo-pyrimidin-2-on-3-yl; bis-ortho-substituted-6-phenyl-pyrrolo-pyrimidin-2-on-3-yl; Difluorotolyl; Hypoxanthine; Imidizopyridinyl; Inosinyl; Isocarbostyrilyl; Isoguanisine; N2-substituted purines; N6-methyl-2-amino-purine; N6-substituted purines; N-alkylated derivative; Napthalenyl; Nitrobenzimidazolyl; Nitroimidazolyl; Nitroindazolyl; Nitropyrazolyl; Nubularine; 06-substituted purines; O-alkylated derivative; ortho-(aminoalkylhydroxy)-6-phenyl-pyrrolo-pyrimidin-2-on-3-yl; ortho-substituted-6-phenyl-pyrrolo-pyrimidin-2-on-3-yl; Oxoformycin TP; para-(aminoalkylhydroxy)-6-phenyl-pyrrolo-pyrimidin-2-on-3-yl; para-substituted-6-phenyl-pyrrolo-pyrimidin-2-on-3-yl; Pentacenyl; Phenanthracenyl; Phenyl; propynyl-7-(aza)indolyl; Pyrenyl; pyridopyrimidin-3-yl; pyridopyrimidin-3-yl, 2-oxo-7-amino-pyridopyrimidin-3-yl; pyrrolo-pyrimidin-2-on-3-yl; Pyrrolopyrimidinyl; Pyrrolopyrizinyl; Stilbenzyl; substituted 1,2,4-triazoles; Tetracenyl; Tubercidine; Xanthine; Xanthosine-5′-TP; 2-thio-zebularine; 5-aza-2-thio-zebularine; 7-deaza-2-amino-purine; pyridin-4-one ribonucleoside; 2-Amino-riboside-TP; Formycin A TP; Formycin B TP; Pyrrolosine TP; 2′-OH-ara-adenosine TP; 2′-OH-ara-cytidine TP; 2′-OH-ara-uridine TP; 2′-OH-ara-guanosine TP; 5-(2-carbomethoxyvinyl)uridine TP; and N6-(19-Amino-pentaoxanonadecyl)adenosine TP.

In some embodiments, RNA molecules (e.g., mRNA molecules) include a combination of at least two (e.g., 2, 3, 4 or more) of the aforementioned modified nucleobases.

In some embodiments, modified nucleobases in RNA molecules (e.g., mRNA molecules) are selected from the group consisting of pseudouridine (ψ), N1-methylpseudouridine (m¹ψ), N1-ethylpseudouridine, 2-thiouridine, 4′-thiouridine, 5-methylcyto sine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine and 2′-O-methyl uridine. In some embodiments, polynucleotides (e.g., RNA polynucleotides, such as mRNA polynucleotides) include a combination of at least two (e.g., 2, 3, 4 or more) of the aforementioned modified nucleobases.

In some embodiments, modified nucleobases in RNA molecules (e.g., mRNA molecules) are selected from the group consisting of 1-methyl-pseudouridine (m¹ψ), 5-methoxy-uridine (mo⁵U), 5-methyl-cytidine (m⁵C), pseudouridine (ψ), α-thio-guanosine and α-thio-adenosine. In some embodiments, polynucleotides include a combination of at least two (e.g., 2, 3, 4 or more) of the aforementioned modified nucleobases.

In some embodiments, RNA molecules (e.g., mRNA molecules) comprise pseudouridine (v) and 5-methyl-cytidine (m⁵C). In some embodiments, RNA molecules (e.g., mRNA molecules) comprise 1-methyl-pseudouridine (m¹ψ). In some embodiments, RNA molecules (e.g., mRNA molecules) comprise 1-methyl-pseudouridine (m¹ψ) and 5-methyl-cytidine (m⁵C). In some embodiments, RNA molecules (e.g., mRNA molecules) comprise 2-thiouridine (s²U). In some embodiments, RNA molecules (e.g., mRNA molecules) comprise 2-thiouridine and 5-methyl-cytidine (m⁵C). In some embodiments, RNA molecules (e.g., mRNA molecules) comprise methoxy-uridine (mo⁵U). In some embodiments, RNA molecules (e.g., mRNA molecules) comprise 5-methoxy-uridine (mo⁵U) and 5-methyl-cytidine (m⁵C). In some embodiments, RNA molecules (e.g., mRNA molecules) comprise 2′-O-methyl uridine. In some embodiments RNA molecules (e.g., mRNA molecules) comprise 2′-O-methyl uridine and 5-methyl-cytidine (m⁵C). In some embodiments, RNA molecules (e.g., mRNA molecules) comprise N6-methyl-adenosine (m⁶A). In some embodiments, RNA molecules (e.g., mRNA molecules) comprise N6-methyl-adenosine (m⁶A) and 5-methyl-cytidine (m⁵C).

In some embodiments, RNA molecules (e.g., mRNA molecules) are uniformly modified (e.g., fully modified, modified throughout the entire sequence) for a particular modification. For example, an RNA molecule can be uniformly modified with 1-methyl-pseudouridine (m¹ψ), meaning that all uridine residues in the mRNA sequence are replaced with 1-methyl-pseudouridine (m¹ψ). Similarly, an RNA molecule can be uniformly modified for any type of nucleoside residue present in the sequence by replacement with a modified residue such as those set forth above.

In some embodiments, the RNA (e.g., mRNA) molecules comprise a 5′UTR element, an optionally codon optimized open reading frame, and a 3′UTR element, a poly(A) sequence and/or a polyadenylation signal wherein the RNA is not chemically modified.

In some embodiments, the modified nucleobase is a modified uracil. Exemplary nucleobases and nucleosides having a modified uracil include pseudouridine (ψ), N1-methyl-pseudouridine (m1^{ψ}), pyridin-4-one ribonucleoside, 5-aza-uridine, 6-aza-uridine, 2-thio-5-aza-uridine, 2-thio-uridine (s²U), 4-thio-uridine (s⁴U), 4-thio-pseudouridine, 2-thio-pseudouridine, 5-hydroxy-uridine (ho⁵U), 5-aminoallyl-uridine, 5-halo-uridine (e.g., 5-iodo-uridineor 5-bromo-uridine), 3-methyl-uridine (m³U), 5-methoxy-uridine (mo⁵U), uridine 5-oxyacetic acid (cmo⁵U), uridine 5-oxyacetic acid methyl ester (mcmo⁵U), 5-carboxymethyl-uridine (cm⁵U), 1-carboxymethyl-pseudouridine, 5-carboxyhydroxymethyl-uridine (chm⁵U), 5-carboxyhydroxymethyl-uridine methyl ester (mchm⁵U), 5-methoxycarbonylmethyl-uridine (mcm⁵U), 5-methoxycarbonylmethyl-2-thio-uridine (mcm⁵s²U), 5-aminomethyl-2-thio-uridine (nm⁵s²U), 5-methylaminomethyl-uridine (mnm⁵U), 5-methylaminomethyl-2-thio-uridine (mnm⁵s²U), 5-methylaminomethyl-2-seleno-uridine (mnm⁵se²U), 5-carbamoylmethyl-uridine (ncm⁵U), 5-carboxymethylaminomethyl-uridine (cmnm⁵U), 5-carboxymethylaminomethyl-2-thio-uridine (cmnm⁵s²U), 5-propynyl-uridine, 1-propynyl-pseudouridine, 5-taurinomethyl-uridine (τm⁵U), 1-taurinomethyl-pseudouridine, 5-taurinomethyl-2-thio-uridine(m⁵s²U), 1-taurinomethyl-4-thio-pseudouridine, 5-methyl-uridine (m⁵U, i.e., having the nucleobase deoxythymine), 1-methyl-pseudouridine (m¹ψ), 5-methyl-2-thio-uridine (m⁵s²U), 1-methyl-4-thio-pseudouridine (m¹s⁴ψ), 4-thio-1-methyl-pseudouridine, 3-methyl-pseudouridine (m³ψ), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m⁵D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, 3-(3-amino-3-carboxypropyl)uridine (acp³U), 1-methyl-3-(3-amino-3-carboxypropyl)pseudouridine (acp³ψ), 5-(isopentenylaminomethyl)uridine (inm⁵U), 5-(isopentenylaminomethyl)-2-thio-uridine (inm⁵s²U), α-thio-uridine, 2′-O-methyl-uridine (Um), 5,2′-O-dimethyl-uridine (msUm), 2′-O-methyl-pseudouridine (Wm), 2-thio-2′-O-methyl-uridine (s²Um), 5-methoxycarbonylmethyl-2′-O-methyl-uridine (mcm⁵Um), 5-carbamoylmethyl-2′-O-methyl-uridine (ncm⁵Um), 5-carboxymethylaminomethyl-2′-O-methyl-uridine (cmnm⁵Um), 3,2′-O-dimethyl-uridine (m³Um), and 5-(isopentenylaminomethyl)-2′-O-methyl-uridine (inm⁵Um), 1-thio-uridine, deoxythymidine, 2′-F-ara-uridine, 2′-F-uridine, 2′-OH-ara-uridine, 5-(2-carbomethoxyvinyl) uridine, and 5-[3-(1-E-propenylamino)]uridine.

In some embodiments, the modified nucleobase is a modified cytosine. Exemplary nucleobases and nucleosides having a modified cytosine include 5-aza-cytidine, 6-aza-cytidine, pseudoisocytidine, 3-methyl-cytidine (m³C), N4-acetyl-cytidine (ac⁴C), 5-formyl-cytidine (f⁵C), N4-methyl-cytidine (m⁴C), 5-methyl-cytidine (m⁵C), 5-halo-cytidine (e.g., 5-iodo-cytidine), 5-hydroxymethyl-cytidine (hm⁵C), 1-methyl-pseudoisocytidine, pyrrolo-cytidine, pyrrolo-pseudoisocytidine, 2-thio-cytidine (s²C), 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, lysidine (k₂C), α-thio-cytidine, 2′-O-methyl-cytidine (Cm), 5,2′-O-dimethyl-cytidine (m⁵Cm), N4-acetyl-2′-O-methyl-cytidine (ac⁴Cm), N4,2′-O-dimethyl-cytidine (m⁴Cm), 5-formyl-2′-O-methyl-cytidine (f⁵Cm), N4,N4,2′-O-trimethyl-cytidine (m⁴2Cm), 1-thio-cytidine, 2′-F-ara-cytidine, 2′-F-cytidine, and 2′-OH-ara-cytidine.

In some embodiments, the modified nucleobase is a modified adenine. Exemplary nucleobases and nucleosides having a modified adenine include 2-amino-purine, 2, 6-diaminopurine, 2-amino-6-halo-purine (e.g., 2-amino-6-chloro-purine), 6-halo-purine (e.g., 6-chloro-purine), 2-amino-6-methyl-purine, 8-azido-adenosine, 7-deaza-adenine, 7-deaza-8-aza-adenine, 7-deaza-2-amino-purine, 7-deaza-8-aza-2-amino-purine, 7-deaza-2,6-diaminopurine, 7-deaza-8-aza-2,6-diaminopurine, 1-methyl-adenosine (m¹A), 2-methyl-adenine (m²A), N6-methyl-adenosine (m⁶A), 2-methylthio-N6-methyl-adenosine (ms²m⁶A), N6-isopentenyl-adenosine (i⁶A), 2-methylthio-N6-isopentenyl-adenosine (ms²i⁶A), N6-(cis-hydroxyisopentenyl)adenosine (io⁶A), 2-methylthio-N6-(cis-hydroxyisopentenyl)adenosine (ms²io⁶A), N6-glycinylcarbamoyl-adenosine (g⁶A), N6-threonylcarbamoyl-adenosine (t⁶A), N6-methyl-N6-threonylcarbamoyl-adenosine (m⁶t6A), 2-methylthio-N6-threonylcarbamoyl-adenosine (ms²g⁶A), N6,N6-dimethyl-adenosine (m⁶2A), N6-hydroxynorvalylcarbamoyl-adenosine (hn⁶A), 2-methylthio-N6-hydroxynorvalylcarbamoyl-adenosine (ms²hn⁶A), N6-acetyl-adenosine (ac⁶A), 7-methyl-adenine, 2-methylthio-adenine, 2-methoxy-adenine, α-thio-adenosine, 2′-O-methyl-adenosine (Am), N6,2′-O-dimethyl-adenosine (m⁶Am), N6,N6,2′-O-trimethyl-adenosine (m⁶2Am), 1,2′-O-dimethyl-adenosine (m¹Am), 2′-O-ribosyladenosine (phosphate) (Ar(p)), 2-amino-N6-methyl-purine, 1-thio-adenosine, 8-azido-adenosine, 2′-F-ara-adenosine, 2′-F-adenosine, 2′-OH-ara-adenosine, and N6-(19-amino-pentaoxanonadecyl)-adenosine.

In some embodiments, the modified nucleobase is a modified guanine. Exemplary nucleobases and nucleosides having a modified guanine include inosine (I), 1-methyl-inosine (m¹1), wyosine (imG), methylwyosine (mimG), 4-demethyl-wyosine (imG-14), isowyosine (imG2), wybutosine (yW), peroxywybutosine (o2yW), hydroxywybutosine (OhyW), undermodified hydroxywybutosine (OhyW*), 7-deaza-guanosine, queuosine (Q), epoxyqueuosine (oQ), galactosyl-queuosine (galQ), mannosyl-queuosine (manQ), 7-cyano-7-deaza-guanosine (preQ₀), 7-aminomethyl-7-deaza-guanosine (preQ₁), archaeosine (G⁺), 7-deaza-8-aza-guanosine, 6-thio-guanosine, 6-thio-7-deaza-guanosine, 6-thio-7-deaza-8-aza-guanosine, 7-methyl-guanosine (m⁷G), 6-thio-7-methyl-guanosine, 7-methyl-inosine, 6-methoxy-guanosine, 1-methyl-guanosine (mG), N2-methyl-guanosine (m²G), N2,N2-dimethyl-guanosine (m²2G), N2,7-dimethyl-guanosine (m^{2,7}G), N2, N2,7-dimethyl-guanosine (m^{2,2,7}G), 8-oxo-guanosine, 7-methyl-8-oxo-guanosine, 1-methyl-6-thio-guanosine, N2-methyl-6-thio-guanosine, N2,N2-dimethyl-6-thio-guanosine, α-thio-guanosine, 2′-O-methyl-guanosine (Gm), N2-methyl-2′-O-methyl-guanosine (m²Gm), N2,N2-dimethyl-2′-O-methyl-guanosine (m²2Gm), 1-methyl-2′-O-methyl-guanosine (mGm), N2,7-dimethyl-2′-O-methyl-guanosine (m²′7Gm), 2′-O-methyl-inosine (Im), 1,2′-O-dimethyl-inosine (m¹Im), 2′-O-ribosylguanosine (phosphate) (Gr(p)), 1-thio-guanosine, 06-methyl-guanosine, 2′-F-ara-guanosine, and 2′-F-guanosine.

The nucleic acid molecules of the present disclosure may be partially or fully modified along the entire length of the molecule. For example, one or more or all or a given type of nucleotide (e.g., purine or pyrimidine, or any one or more or all of A, G, U, C) may be uniformly modified in a nucleic acid molecule of the disclosure, or in a given predetermined sequence region thereof (e.g., in the mRNA including or excluding the polyA tail). In some embodiments, all nucleotides X in a nucleic acid molecule of the present disclosure (or in a given sequence region thereof) are modified nucleotides, wherein X may any one of nucleotides A, G, U, C, or any one of the combinations A+G, A+U, A+C, G+U, G+C, U+C, A+G+U, A+G+C, G+U+C or A+G+C.

The nucleic acid molecule may contain from about 1% to about 100% modified nucleotides (either in relation to overall nucleotide content, or in relation to one or more types of nucleotide, i.e., any one or more of A, G, U or C) or any intervening percentage (e.g., from 1% to 20%, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%). Any remaining percentage is accounted for by the presence of unmodified A, G, U, or C.

For example, in some embodiments, at least 10%, at least 20%, at least 30%, at least 40%, at least 50%, at least 60%, at least 70%, at least 80%, at least 90%, at least 95%, at least 96%, at least 97%, at least 98%, or at least 99% of the uridines in an RNA molecule (e.g., an mRNA molecule) are N1-methyl-pseudouridine (m1^{ψ}). In some embodiments, at least 1, 2, 3, 4, 5, 6, 7, 8, 9, 10, 15, 20, 25, 30, 35, 40, 45, 50, 60, 70, 80, 90, 100, 150, 200, 250, 300, 350, 400, 450, or 500 uridines in an RNA molecule (e.g., an mRNA molecule) are N1-methyl-pseudouridine (m¹ψ). In some embodiments, all of the uridines in an RNA molecule (e.g., an mRNA molecule) are N1-methyl-pseudouridine (m¹ψ).

In some embodiments, the mRNA is non-replicating mRNA. In other embodiments, the mRNA is self-amplifying mRNA. Self-amplifying mRNA may be based on an alphavirus genome, which contains the genes encoding the alphavirus RNA replication machinery, but lacks the genes encoding the viral structural proteins required to make an infectious alphavirus particle. See Geall A J et al., 2012, Proc Natl Acad Sci USA 109(36): 14604-14609. The structural protein genes of the alphavirus may be replaced with one or more nucleic acid molecules encoding two or more thanotransmission polypeptides, which are abundantly expressed from a subgenomic mRNA in the cytoplasm of cells transfected with these self-amplifying RNAs. The self-amplifying mRNAs may be produced in vitro by an enzymatic transcription reaction from a linear pDNA template using a T7 RNA polymerase, thereby avoiding safety concerns and complex manufacturing issues associated with cell culture production of live viral vaccines, recombinant subunit proteins, and viral vectors. After immunization, replication and amplification of the mRNA molecule occurs exclusively in the cytoplasm of the transfected cells, thereby eliminating risks of genomic integration and cell transformation. See Brito L A, et al. 2015, Adv Genet. 89:179-233; Perri S, et al. 2003, J Virol. 77:10394-403; and Geall A J, et al., 2012, Proc Natl Acad Sci USA. 109:14604-9.

The full length mRNA of the self-amplifying mRNA is substantially larger (approximately 9-10 kb for alphavirus systems) than in non-replicating mRNAs but contains the same essential elements such as a cap, 5′ and 3′ UTRs, and poly A tail as described above. The DNA encoding the self-amplifying mRNA comprises a sub-genomic promoter and a large ORF encoding nonstructural viral proteins which, following delivery of the DNA into the cytosol, are transcribed in four functional components (nsP1, nsP2, nsP3, and nsp4) by the encoded RNA-dependent RNA polymerase (RDRP) (see Iavarone C, et al., 2017, Expert Rev Vaccines 16:871-81). RDRP then produces a negative-sense copy of the genome which serves as a template for two positive strand RNA molecules: the genomic mRNA and a shorter sub-genomic mRNA. This sub-genomic mRNA is transcribed at very high levels, allowing the amplification of mRNA encoding the polypeptide of choice.

In some embodiments, the mRNAs may be codon optimized to modulate their stability. For example, in some embodiments, a codon optimized mRNA has increased stability relative to a corresponding mRNA that is not codon optimized. In some embodiments, a codon optimized mRNA has decreased stability relative to a corresponding mRNA that is not codon optimized. For example, the mRNA encoding the thanotransmission polypeptide may be codon optimized to increase it stability. Methods of codon optimizing mRNA to modulate stability are known in the art and are described, for example, in Bicknell A A et al., 2017, Biochem Soc Trans. 45(2):339-351; Radhakrishnan A, et al, 2016, J Mol Biol. 428(18):3558-3564; Chen Y H, et al., 2016, Trends Genet. 2016; 32(11):687-688; and Hanson G, et al., 2018, Nat Rev Mol Cell Biol. 19(1):20-30.

The mRNA may comprise one or more modified nucleotides to modulate its stability. In some embodiments, the one or more modified nucleotides increase stability of the mRNA relative to a corresponding mRNA that does not comprise the one or more modified nucleotides. In some embodiments, the one or more modified nucleotides decrease stability of the mRNA relative to a corresponding mRNA that does not comprise the one or more modified nucleotides. Suitable modified nucleotides include, but are not limited to, N6-methyladenosine (m6A), N6,2′-O-dimethyladenosine (m6Am), 5-methylcytidine (m5C), inosine (I), pseudouridine (ψ), Ni-methyladenosine (m1A), 5-hydroxylmethylcytidine (hm5C), 2′-O-methylation (Nm), and N4-Acetylcytidine. See Roundtree I A, et al., 2017, Cell 169(7):1187-1200; and Li X, et al., 2019, Biochemistry 58(12):1553-1554.

The mRNA may comprises a protein binding site in a 3′-UTR of the mRNA. The protein binding site may decreases stability of the mRNA. In some embodiments, the protein binding site is a Staufen1 (STAU1)-mediated binding site (SBS). See Park E, et al., 2013, Wiley Interdiscip Rev RNA. 4(4):423-435; and Chen Y H, et al., 2016, Trends Genet. 32(11):687-688. Staufen1 (STAU1)-mediated mRNA decay (SMD) is an mRNA degradation process in mammalian cells that is mediated by the binding of STAU1 to a STAU1-binding site (SBS) within the 3′-untranslated region (3′-UTR) of target mRNAs. During SMD, STAU1, a double-stranded (ds) RNA-binding protein, recognizes dsRNA structures formed either by intramolecular base pairing of 3′-UTR sequences or by intermolecular base pairing of 3′-UTR sequences with a long-noncoding RNA (lncRNA) via partially complementary Alu elements. STAU1 interacts directly with the ATP-dependent RNA helicase UPF1, a key SMD factor, enhancing its helicase activity to promote effective STAU1-mediated mRNA decay.

MicroRNAs

MicroRNAs (miRNAs) are 19-25 nucleotide long noncoding RNAs that bind to the 3′UTR of nucleic acid molecules and down-regulate gene expression, either by reducing nucleic acid molecule stability or by inhibiting translation. Several microRNAs are known in the art and are described, for example, in U.S. Ser. No. 10/072,057, US2005/0261218 and US2005/0059005, the contents of each of which are incorporated by reference herein in their entirety.

As used herein, the term “microRNA binding site” refers to a microRNA target site or a microRNA recognition site, or any nucleotide sequence to which a microRNA binds or associates.

A microRNA sequence comprises a “seed” region, i.e., positions 2-8 of the mature microRNA, which has perfect complementarity to the miRNA binding site. The term “seed-complementary site” as used herein refers to the region of the miRNA binding site that is complementary to the seed region of the miRNA. In some embodiments, the nucleotides of the microRNA seed region have complete complementarity with the seed-complementary site of the miRNA binding site.

By engineering microRNA binding sites into the 3′ UTR of mRNAs encoding thanotransmission polypeptides as described herein, one can target the mRNA for degradation or reduced translation in cells in which the corresponding microRNA is present. This process will reduce off target effects upon recombinant nucleic acid molecule delivery. Identification of microRNA, microRNA binding sites, and their expression patterns and role in biology have been reported (Bonauer et al., Curr Drug Targets 2010 11:943-949; Anand and Cheresh Curr Opin Hematol 2011 18:171-176; Contreras and Rao Leukemia 2012 26:404-413 (2011 Dec. 20. doi: 10.1038/Ieu.2011.356); Bartel Cell 2009 136:215-233; Landgraf et al, Cell, 2007 129:1401-1414; Gentner and Naldini, Tissue Antigens. 2012 80:393-403 and all references therein; each of which is incorporated herein by reference in its entirety).

For example, if the mRNA is not intended to be delivered to the liver but ends up there, then miR-122 (e.g., miR-122-5p), a microRNA abundant in liver, can inhibit the expression of the gene of interest if one or multiple binding sites of miR-122 are engineered into the mRNA, e.g., the 3′ UTR of the mRNA. One or more binding sites for different microRNAs can be added to an mRNA to further decrease the longevity, stability, and protein translation of an mRNA in a particular cell or tissue.

In some embodiments, the recombinant nucleic acid molecule encoding IL-12 and/or one or more thanotransmission polypeptides comprises one or more miRNA binding sites, or one or more polynucleotides encoding an miRNA binding site. For example, in some embodiments, the recombinant nucleic acid molecule encoding IL-12 and/or one or more thanotransmission polypeptides is a DNA molecule, and comprises one or more polynucleotides encoding an miRNA binding site. In some embodiments, the recombinant nucleic acid molecule encoding IL-12 and/or one or more thanotransmission polypeptides comprises polynucleotides encoding at least two different miRNA binding sites, e.g., at least 2, 3, 4 or 5 different miRNA binding sites. In some embodiments, the nucleic acid molecule encoding IL-12 and/or one or more thanotransmission polypeptides is an RNA molecule (e.g., an mRNA molecule) and comprises one or more miRNA binding sites. In some embodiments, the RNA molecule comprises at least two different miRNA binding sites, e.g., at least 2, 3, 4 or 5 different miRNA binding sites. In some embodiments, the two different miRNA binding sites are miR-122 (e.g., miR-122-5p) and miR-142 (e.g., miR-142-3p).

Regulation of expression in multiple tissues can be accomplished through introduction of one or several microRNA binding sites into an mRNA. Examples of tissues where microRNA are known to regulate mRNA, and thereby protein expression, include, but are not limited to, liver (miR-122), muscle (miR-133, miR-206, miR-208), endothelial cells (miR-17-92, miR-126), myeloid cells (miR-142-3p, miR-142-5p, miR-16, miR-21, miR-223, miR-24, miR-27), adipose tissue (let-7, miR-30c), heart (miR-Id, miR-149), kidney (miR-192, miR-194, miR-204), and lung epithelial cells (let-7, miR-133, miR-126).

Specifically, microRNAs are known to be differentially expressed in immune cells (also called hematopoietic cells), such as antigen presenting cells (APCs) (e.g. dendritic cells and macrophages), macrophages, monocytes, B lymphocytes, T lymphocytes, granulocytes, natural killer cells, etc. Immune cell specific microRNAs are involved in immunogenicity, autoimmunity, the immune-response to infection, inflammation, as well as unwanted immune response after gene therapy and tissue/organ transplantation. Immune cells specific microRNAs also regulate many aspects of development, proliferation, differentiation and apoptosis of hematopoietic cells (immune cells). For example, miR-142 and miR-146 are exclusively expressed in the immune cells, and are particularly abundant in myeloid dendritic cells. It was demonstrated in the art that the immune response to exogenous nucleic acid molecules was shut-off by adding miR-142 binding sites to the 3′-UTR of the delivered gene construct, enabling more stable gene transfer in tissues and cells. miR-142 efficiently degrades the exogenous mRNA in antigen presenting cells and suppresses cytotoxic elimination of transduced cells (Annoni A et al., blood, 2009, 114, 5152-5161; Brown B D, et al., Nat med. 2006, 12(5), 585-591; Brown B D, et al., blood, 2007, 110(13): 4144-4152, each of which is incorporated herein by reference in its entirety).

An antigen-mediated immune response can refer to an immune response triggered by foreign antigens, which, when entering an organism, are processed by the antigen presenting cells and displayed on the surface of the antigen presenting cells. T cells can recognize the presented antigen and induce a cytotoxic elimination of cells that express the antigen.

Introducing the miR-142 binding site into the 3′-UTR of an mRNA can selectively repress the gene expression in the antigen presenting cells through miR-142 mediated mRNA degradation, limiting antigen presentation in APCs (e.g. dendritic cells) and thereby preventing antigen-mediated immune response after the delivery of the polynucleotides. The polynucleotides are therefore stably expressed in target tissues or cells without triggering cytotoxic elimination.

In one embodiment, microRNAs binding sites that are known to be expressed in immune cells, in particular, the antigen presenting cells, can be engineered into the polynucleotide to suppress the expression of the sensor-signal polynucleotide in APCs through microRNA mediated RNA degradation, subduing the antigen-mediated immune response, while the expression of the polynucleotide is maintained in non-immune cells where the immune cell specific microRNAs are not expressed.

Immune cell specific microRNAs include, but are not limited to, hsa-let-7a-2-3p, hsa-let-7a-3p, hsa-7a-5p, hsa-let-7c, hsa-let-7e-3p, hsa-let-7e-5p, hsa-let-7g-3p, hsa-let-7g-5p, hsa-let-7i-3p, hsa-let-7i-5p, miR-10a-3p, miR-10a-5p, miR-1184, hsa-let-7f-1-3p, hsa-let-7f-2-5p, hsa-let-7f-5p, miR-125b-1-3p, miR-125b-2-3p, miR-125b-5p, miR-1279, miR-130a-3p, miR-130a-5p, miR-132-3p, miR-132-5p, miR-142-3p, miR-142-5p, miR-143-3p, miR-143-5p, miR-146a-3p, miR-146a-5p, miR-146b-3p, miR-146b-5p, miR-147a, miR-147b, miR-148a-5p, miR-148a-3p, miR-150-3p, miR-150-5p, miR-151b, miR-155-3p, miR-155-5p, miR-15a-3p, miR-15a-5p, miR-15b-5p, miR-15b-3p, miR-16-1-3p, miR-16-2-3p, miR-16-5p, miR-17-5p, miR-181a-3p, miR-181a-5p, miR-181a-2-3p, miR-182-3p, miR-182-5p, miR-197-3p, miR-197-5p, miR-21-5p, miR-21-3p, miR-214-3p, miR-214-5p, miR-223-3p, miR-223-5p, miR-221-3p, miR-221-5p, miR-23b-3p, miR-23b-5p, miR-24-1-5p, miR-24-2-5p, miR-24-3p, miR-26a-1-3p, miR-26a-2-3p, miR-26a-5p, miR-26b-3p, miR-26b-5p, miR-27a-3p, miR-27a-5p, miR-27b-3p, miR-27b-5p, miR-28-3p, miR-28-5p, miR-2909, miR-29a-3p, miR-29a-5p, miR-29b-1-5p, miR-29b-2-5p, miR-29c-3p, miR-29c-5p, miR-30e-3p, miR-30e-5p, miR-331-5p, miR-339-3p, miR-339-5p, miR-345-3p, miR-345-5p, miR-346, miR-34a-3p, miR-34a-5p, miR-363-3p, miR-363-5p, miR-372, miR-377-3p, miR-377-5p, miR-493-3p, miR-493-5p, miR-542, miR-548b-5p, miR548c-5p, miR-548i, miR-548j, miR-548n, miR-574-3p, miR-598, miR-718, miR-935, miR-99a-3p, miR-99a-5p, miR-99b-3p and miR-99b-5p. Furthermore, novel miroRNAs are discovered in the immune cells in the art through micro-array hybridization and microtome analysis (Jima D et al, Blood, 2010, 116:e118-e127; Vaz C et al., BMC Genomics, 2010, 11, 288, the content of each of which is incorporated herein by reference in its entirety.)

MicroRNAs that are known to be expressed in the liver include, but are not limited to, miR-107, miR-122-3p, miR-122-5p, miR-1228-3p, miR-1228-5p, miR-1249, miR-129-5p, miR-1303, miR-151a-3p, miR-151a-5p, miR-152, miR-194-3p, miR-194-5p, miR-199a-3p, miR-199a-5p, miR-199b-3p, miR-199b-5p, miR-296-5p, miR-557, miR-581, miR-939-3p, miR-939-5p. MicroRNA binding sites from any liver specific microRNA can be introduced to the recombinant nucleic acid molecules to regulate the expression of the polynucleotides in the liver. Liver specific microRNAs binding sites can be engineered alone or further in combination with immune cells (e.g. APCs) microRNA binding sites in order to prevent immune reaction against protein expression in the liver.

MicroRNAs that are known to be expressed in the lung include, but are not limited to, let-7a-2-3p, let-7a-3p, let-7a-5p, miR-126-3p, miR-126-5p, miR-127-3p, miR-127-5p, miR-130a-3p, miR-130a-5p, miR-130b-3p, miR-130b-5p, miR-133a, miR-133b, miR-134, miR-18a-3p, miR-18a-5p, miR-18b-3p, miR-18b-5p, miR-24-1-5p, miR-24-2-5p, miR-24-3p, miR-296-3p, miR-296-5p, miR-32-3p, miR-337-3p, miR-337-5p, miR-381-3p, miR-381-5p.

MicroRNA binding sites from any lung specific microRNA can be introduced to the recombinant nucleic acid molecules to regulate the expression in the lung. Lung specific microRNAs binding sites can be engineered alone or further in combination with immune cells (e.g. APCs) microRNA binding sites in order to prevent an immune reaction against protein expression in the lung.

MicroRNAs that are known to be expressed in the heart include, but are not limited to, miR-1, miR-133a, miR-133b, miR-149-3p, miR-149-5p, miR-186-3p, miR-186-5p, miR-208a, miR-208b, miR-210, miR-296-3p, miR-320, miR-451a, miR-451b, miR-499a-3p, miR-499a-5p, miR-499b-3p, miR-499b-5p, miR-744-3p, miR-744-5p, miR-92b-3p and miR-92b-5p. MicroRNA binding sites from any heart specific microRNA can be introduced to the recombinant nucleic acid molecules to reduce expression in the heart. Heart specific microRNAs binding sites can be engineered alone or further in combination with immune cells (e.g. APCs) microRNA binding sites to prevent an immune reaction against protein expression in the heart.

MicroRNAs that are known to be expressed in the nervous system include, but are not limited to, miR-124-5p, miR-125a-3p, miR-125a-5p, miR-125b-1-3p, miR-125b-2-3p, miR-125b-5p, miR-1271-3p, miR-1271-5p, miR-128, miR-132-5p, miR-135a-3p, miR-135a-5p, miR-135b-3p, miR-135b-5p, miR-137, miR-139-5p, miR-139-3p, miR-149-3p, miR-149-5p, miR-153, miR-181c-3p, miR-181c-5p, miR-183-3p, miR-183-5p, miR-190a, miR-190b, miR-212-3p, miR-212-5p, miR-219-1-3p, miR-219-2-3p, miR-23a-3p, miR-23a-5p, miR-30a-5p, miR-30b-3p, miR-30b-5p, miR-30c-1-3p, miR-30c-2-3p, miR-30c-5p, miR-30d-3p, miR-30d-5p, miR-329, miR-342-3p, miR-3665, miR-3666, miR-380-3p, miR-380-5p, miR-383, miR-410, miR-425-3p, miR-425-5p, miR-454-3p, miR-454-5p, miR-483, miR-510, miR-516a-3p, miR-548b-5p, miR-548c-5p, miR-571, miR-7-1-3p, miR-7-2-3p, miR-7-5p, miR-802, miR-922, miR-9-3p and miR-9-5p. MicroRNAs enriched in the nervous system further include those specifically expressed in neurons, including, but not limited to, miR-132-3p, miR-132-3p, miR-148b-3p, miR-148b-5p, miR-151a-3p, miR-151a-5p, miR-212-3p, miR-212-5p, miR-320b, miR-320e, miR-323a-3p, miR-323a-5p, miR-324-5p, miR-325, miR-326, miR-328, miR-922 and those specifically expressed in glial cells, including, but not limited to, miR-1250, miR-219-1-3p, miR-219-2-3p, miR-219-5p, miR-23a-3p, miR-23a-5p, miR-3065-3p, miR-3065-5p, miR-30e-3p, miR-30e-5p, miR-32-5p, miR-338-5p, miR-657. MicroRNA binding sites from any CNS specific microRNA can be introduced to the recombinant nucleic acid molecules to reduce expression in the nervous system. Nervous system specific microRNAs binding sites can be engineered alone or further in combination with immune cells (e.g. APCs) microRNA binding sites in order to prevent immune reaction against protein expression in the nervous system.

MicroRNAs that are known to be expressed in the pancreas include, but are not limited to, miR-105-3p, miR-105-5p, miR-184, miR-195-3p, miR-195-5p, miR-196a-3p, miR-196a-5p, miR-214-3p, miR-214-5p, miR-216a-3p, miR-216a-5p, miR-30a-3p, miR-33a-3p, miR-33a-5p, miR-375, miR-7-1-3p, miR-7-2-3p, miR-493-3p, miR-493-5p and miR-944. MicroRNA binding sites from any pancreas specific microRNA can be introduced to the recombinant nucleic acid molecules to reduce expression in the pancreas. Pancreas specific microRNAs binding sites can be engineered alone or further in combination with immune cells (e.g. APCs) microRNA binding sites in order to prevent an immune reaction against protein expression in the pancreas.

MicroRNAs that are known to be expressed in the kidney further include, but are not limited to, miR-122-3p, miR-145-5p, miR-17-5p, miR-192-3p, miR-192-5p, miR-194-3p, miR-194-5p, miR-20a-3p, miR-20a-5p, miR-204-3p, miR-204-5p, miR-210, miR-216a-3p, miR-216a-5p, miR-296-3p, miR-30a-3p, miR-30a-5p, miR-30b-3p, miR-30b-5p, miR-30c-1-3p, miR-30c-2-3p, miR30c-5p, miR-324-3p, miR-335-3p, miR-335-5p, miR-363-3p, miR-363-5p and miR-562. MicroRNA binding sites from any kidney specific microRNA can be introduced into the recombinant nucleic acid molecules to reduce expression in the kidney. Kidney specific microRNAs binding sites can be engineered alone or further in combination with immune cells (e.g. APCs) microRNA binding sites to prevent an immune reaction against protein expression in the kidney.

MicroRNAs that are known to be expressed in muscle further include, but are not limited to, let-7g-3p, let-7g-5p, miR-1, miR-1286, miR-133a, miR-133b, miR-140-3p, miR-143-3p, miR-143-5p, miR-145-3p, miR-145-5p, miR-188-3p, miR-188-5p, miR-206, miR-208a, miR-208b, miR-25-3p and miR-25-5p. MicroRNA binding sites from any muscle specific microRNA can be introduced into the recombinant nucleic acid molecules to reduce expression of the polynucleotide in the muscle. Muscle specific microRNAs binding sites can be engineered alone or further in combination with immune cells (e.g. APCs) microRNA binding sites to prevent an immune reaction against protein expression in the muscle.

MicroRNAs are differentially expressed in different types of cells, such as endothelial cells, epithelial cells and adipocytes. For example, microRNAs that are expressed in endothelial cells include, but are not limited to, let-7b-3p, let-7b-5p, miR-100-3p, miR-100-5p, miR-101-3p, miR-101-5p, miR-126-3p, miR-126-5p, miR-1236-3p, miR-1236-5p, miR-130a-3p, miR-130a-5p, miR-17-5p, miR-17-3p, miR-18a-3p, miR-18a-5p, miR-19a-3p, miR-19a-5p, miR-19b-1-5p, miR-19b-2-5p, miR-19b-3p, miR-20a-3p, miR-20a-5p, miR-217, miR-210, miR-21-3p, miR-21-5p, miR-221-3p, miR-221-5p, miR-222-3p, miR-222-5p, miR-23a-3p, miR-23a-5p, miR-296-5p, miR-361-3p, miR-361-5p, miR-421, miR-424-3p, miR-424-5p, miR-513a-5p, miR-92a-1-5p, miR-92a-2-5p, miR-92a-3p, miR-92b-3p and miR-92b-5p. Many novel microRNAs are discovered in endothelial cells from deep-sequencing analysis (Voellenkle C et al., RNA, 2012, 18, 472-484, herein incorporated by reference in its entirety) microRNA binding sites from any endothelial cell specific microRNA can be introduced into the recombinant nucleic acid molecules to reduce expression in the endothelial cells in various conditions.

For further example, microRNAs that are expressed in epithelial cells include, but are not limited to, let-7b-3p, let-7b-5p, miR-1246, miR-200a-3p, miR-200a-5p, miR-200b-3p, miR-200b-5p, miR-200c-3p, miR-200c-5p, miR-338-3p, miR-429, miR-451a, miR-451b, miR-494, miR-802 and miR-34a, miR-34b-5p, miR-34c-5p, miR-449a, miR-449b-3p, miR-449b-5p specific in respiratory ciliated epithelial cells; let-7 family, miR-133a, miR-133b, miR-126 specific in lung epithelial cells; miR-382-3p, miR-382-5p specific in renal epithelial cells and miR-762 specific in comeal epithelial cells. MicroRNA binding sites from any epithelial cell specific MicroRNA can be introduced into the recombinant nucleic acid molecules to reduce expression in the epithelial cells in various conditions.

One or more microRNA binding sites can be engineered into the 3′ UTR of the recombinant nucleic acid molecules encoding thanotransmission polypeptides described herein. For example, at least two, at least three, at least four, at least five, at least six, at least seven, at least eight, at least nine, at least ten or more microRNA binding sites may be engineered into the 3′ UTR of the recombinant nucleic acid molecules encoding thanotransmission polypeptides described herein. In some embodiments, the microRNA binding sites may be the same or may be different microRNA binding sites. In some embodiments, the microRNA binding sites target the same or different tissues in the body. As a non-limiting example, through the introduction of tissue-, cell-type-, or disease-specific microRNA binding sites in the 3′ UTR of an mRNA, the degree of expression in specific cell types (e.g. hepatocytes, myeloid cells, endothelial cells, etc.) can be reduced. In some embodiments, the 3′ UTR comprises a miR-122 binding site, e.g., an miR-122-5p binding site. In some embodiments, the 3′ UTR comprises a miR-142 binding site, e.g., an miR-142-3p binding site. In some embodiments, the 3′ UTR comprises a miR-122 binding site (e.g., an miR-122-5p binding site) and a miR-142 binding site (e.g., an miR-142-3p binding site).

In one embodiment, a microRNA binding site can be engineered near the 5′ terminus of the 3′-UTR, about halfway between the 5′ terminus and 3′ terminus of the 3′-UTR and/or near the 3′ terminus of the 3′-UTR. As a non-limiting example, a microRNA site may be engineered near the 5′ terminus of the 3′-UTR and about halfway between the 5′ terminus and 3′ terminus of the 3′-UTR. As another non-limiting example, a microRNA site may be engineered near the 3′ terminus of the 3′-UTR and about halfway between the 5′ terminus and 3′ terminus of the 3′-UTR. As yet another non-limiting example, a microRNA site may be engineered near the 5′ terminus of the 3′-UTR and near the 3′ terminus of the 3′-UTR.

In some embodiments, the 3′-UTR comprises two different microRNA binding sites. The microRNA binding sites may be complete microRNA binding sites, microRNA seed sequences and/or microRNA binding site sequences without the seed sequence.

In some embodiments, the 5′-UTR may comprise at least one microRNA sequence.

In one embodiment, a recombinant nucleic acid molecule as described herein may be engineered to include at least one microRNA in order to dampen the antigen presentation by antigen presenting cells. The microRNA may be the complete microRNA sequence, the microRNA seed sequence, the microRNA sequence without the seed or a combination thereof. As a non-limiting example, the microRNA incorporated into the nucleic acid may be specific to the hematopoietic system. As another non-limiting example, the microRNA incorporated into the nucleic acid of the invention to dampen antigen presentation is miR-142-3p.

In one embodiment, a nucleic acid may be engineered to include microRNA sites that are expressed in different tissues of a subject. As a non-limiting example, a recombinant nucleic acid molecule as described herein may be engineered to include miR-192 and miR-122 to regulate expression in the liver and kidneys of a subject. In another embodiment, a recombinant nucleic acid molecule as described herein may be engineered to include more than one microRNA sites for the same tissue. For example, a recombinant nucleic acid molecule as described herein may be engineered to include miR-17-92 and miR-126 to regulate expression in endothelial cells of a subject.

In one embodiment, the therapeutic window and or differential expression associated with the thanotransmission polypeptide encoded by the recombinant nucleic acid molecule may be altered. For example, polynucleotides may be designed whereby the thanotransmission polypeptide is more highly expressed in cancer cells by virtue of the miRNA signature of those cells. For example, where a cancer cell expresses a lower level of a particular miRNA relative to other cell types, the polynucleotide encoding the binding site for that miRNA (or miRNAs) would be more highly expressed in the cancer cell. Neighboring noncancer cells, harboring a higher expression of the same miRNA would be less affected by the encoded thanotransmission polypeptide, as the polynucleotide would be expressed at a lower level due to the effects of the miRNA binding to the binding site in the 3′-UTR.

Suitable microRNA binding sites for incorporation into the recombinant nucleic acid molecules described herein may be selected by methods known in the art. For example, transfection experiments can be conducted in relevant cell lines, using recombinant nucleic acid molecules engineered to include one or more miRNA binding sites, and protein production can be assayed at various time points post-transfection. Protein expression levels may be determined by using an ELISA kit to the relevant protein produced at 6 hr, 12 hr, 24 hr, 48 hr, 72 hr and 7 days post-transfection. In vivo experiments can also be conducted using microRNA-binding site-engineered molecules to examine changes in tissue-specific expression of formulated alternative nucleic acids, enhanced alternative RNA or ribonucleic acids.

In some embodiments, a recombinant nucleic acid molecule as described herein can be designed to incorporate microRNA binding sites that either have 100% sequence identity to known miRNA binding sites or have less than 100% sequence identity to known miRNA binding sites. For example, in some embodiments, the microRNA binding site has at least 70%, at least 75%, at least 80%, at least 85%, at least 90%, at least 95%, at least 96%, at least 97%, at least 98%, or at least 99% sequence identity to an miRNA binding site disclosed herein. In some embodiments, the seed-complementary site of the miRNA binding site can be partially mutated to decrease microRNA binding affinity, e.g., to attenuate downmodulation of the mRNA transcript. In essence, the degree of match or mis-match between the seed-complementary site of the miRNA binding site and the seed region of the microRNA seed can act as a rheostat to more finely tune the ability of the microRNA to modulate protein expression. In addition, mutation of the miRNA binding site outside of the seed-complementary site may also impact the ability of a microRNA to modulate protein expression.

In some embodiments, an miRNA binding site may be incorporated into the loop of a stem loop. In some embodiments, an miRNA binding site may be incorporated into the 5′ or 3′ stem of the stem loop.

In some embodiments, the composition comprising one or more mRNAs encoding thanotransmission polypeptides further comprises a microRNA (miRNA) or a polynucleotide encoding a miRNA. The miRNA may decrease stability of the mRNA. In some embodiments, the miRNA is complementary to the mRNA encoding the thanotransmission polypeptide. The miRNA may be co-expressed with the mRNA encoding the thanotransmission polypeptide.

In certain aspects the disclosure relates to an RNA molecule comprising: a) a first polynucleotide encoding TRIF or a variant thereof, b) a second polynucleotide encoding an additional polypeptide selected from the group consisting of RIPK3, Gasdermin E, vICA, Npro, A238L, vMLKL, and variants thereof, and a dominant negative variant of IKBa; c) one or more 3′ untranslated regions (3′ UTRs), wherein the one or more 3′ UTRs is operably linked to the first polynucleotide or the second polynucleotide; and d) one or more microRNA (miRNA) binding sites comprised within the one or more 3′UTRs.

In certain aspects the disclosure relates to an RNA molecule comprising: a) a first polynucleotide encoding IL-12 or a variant thereof, b) a second polynucleotide encoding an additional polypeptide selected from the group consisting of TRIF, RIPK3, Gasdermin E, vICA, Npro, A238L, vMLKL, and variants thereof, and a dominant negative variant of IKBa; c) one or more 3′ untranslated regions (3′ UTRs), wherein the one or more 3′ UTRs is operably linked to the first polynucleotide or the second polynucleotide; and d) one or more microRNA (miRNA) binding sites comprised within the one or more 3′UTRs.

In certain aspects the disclosure relates to an RNA molecule comprising: a) a first polynucleotide encoding TRIF or a variant thereof, b) a second polynucleotide encoding an additional polypeptide selected from the group consisting of RIPK3, Gasdermin E, vICA, Npro, A238L, vMLKL, and variants thereof, and a dominant negative variant of IKBa; and c) one or more microRNA (miRNA) binding sites operably linked to the first polynucleotide or the second polynucleotide, wherein the one or more miRNA binding sites comprise a polynucleotide selected from SEQ ID NO: 36 and SEQ ID NO: 37.

In certain aspects the disclosure relates to an RNA molecule comprising: a) a first polynucleotide encoding IL-12 or a variant thereof, b) a second polynucleotide encoding an additional polypeptide selected from the group consisting of TRIF, RIPK3, Gasdermin E, vICA, Npro, A238L, vMLKL, and variants thereof, and a dominant negative variant of IKBa; and c) one or more microRNA (miRNA) binding sites operably linked to the first polynucleotide or the second polynucleotide, wherein the one or more miRNA binding sites comprise a polynucleotide selected from SEQ ID NO: 36 and SEQ ID NO: 37.

In certain aspects the disclosure relates to a recombinant RNA molecule comprising: a) a first polynucleotide encoding TRIF or a variant thereof, b) a second polynucleotide encoding an additional polypeptide selected from the group consisting of RIPK3, Gasdermin E, vICA, Npro, A238L, vMLKL, and variants thereof, and a dominant negative variant of IKBa; and c) one or more 5′ untranslated regions (5′ UTRs) operably linked to the first polynucleotide or the second polynucleotide, wherein the 5′ UTR comprises SEQ ID NO: 33.

In certain aspects the disclosure relates to a recombinant RNA molecule comprising: a) a first polynucleotide encoding IL-12 or a variant thereof, b) a second polynucleotide encoding an additional polypeptide selected from the group consisting of TRIF, RIPK3, Gasdermin E, vICA, Npro, A238L, vMLKL, and variants thereof, and a dominant negative variant of IKBa; and c) one or more 5′ untranslated regions (5′ UTRs) operably linked to the first polynucleotide or the second polynucleotide, wherein the 5′ UTR comprises SEQ ID NO: 33.

In certain aspects the disclosure relates to an RNA molecule comprising: a) a 5′ cap structure; b) a 5′ UTR; c) a first polynucleotide encoding TRIF or a variant thereof, d) a second polynucleotide encoding an additional polypeptide selected from the group consisting of RIPK3, Gasdermin E, vICA, Npro, A238L, vMLKL, and variants thereof, and a dominant negative variant of IKBa; e) a 3′ untranslated region (3′ UTR); and f) one or more microRNA (miRNA) binding sites comprised within the 3′UTR, wherein the one or more miRNA binding regions comprise a polynucleotide selected from SEQ ID NO: 36 and SEQ ID NO: 37.

In certain aspects the disclosure relates to an RNA molecule comprising: a) a 5′ cap structure; b) a 5′ UTR; c) a first polynucleotide encoding IL-12 or a variant thereof; d) a second polynucleotide encoding an additional polypeptide selected from the group consisting of TRIF, RIPK3, Gasdermin E, vICA, Npro, A238L, vMLKL, and variants thereof, and a dominant negative variant of IKBa; e) a 3′ untranslated region (3′ UTR); and f) one or more microRNA (miRNA) binding sites comprised within the 3′UTR, wherein the one or more miRNA binding regions comprise a polynucleotide selected from SEQ ID NO: 36 and SEQ ID NO: 37.

Lipid Nanoparticles (LNPs)

The recombinant nucleic acid molecules (e.g., mRNA molecules) encoding IL-12 and/or one or more thanotransmission polypeptides may be delivered to a subject with synthetic delivery vehicles, such as lipid nanoparticles. Lipid nanoparticles for mRNA molecule delivery are known in the art and are described, for example, in Reichmuth A M, et al., 2016, Ther Deliv. 7(5):319-334; Geall A J, et al., 2012, Proc Natl Acad Sci USA. 109:14604-9; and U.S. Pat. No. 10,702,600, each of which is incorporated by reference herein in its entirety. Suitable lipids and lipid complexes for use in lipid nanoparticles include, but are not limited to, DLinDMA: 1,2-dilinoleyloxy-3-dimethylaminopropane; DOPE: 1,2-dioleoyl-sn-glycero-3-phosphoethanolamine; DOTAP: 1,2-Dioleyl-3-trimethylammonium-propane chloride salt; DSPC: 1,2-Diastearoyl-sn-glycero-3-phosphocholine; Histidylated lipoplex: PEGylated derivative of histidylated polylysine and L-histidine-(N,N-di-n-hexadecylamine)ethylamide liposomes; HVJ-liposome: liposome with fusion proteins derived from the hemagglutinating virus of Japan (HVJ); Man11-LPR100: Mannosylated and histidylated lipopolyplexes (Man11-LPR100) obtained by adding mannosylated and histidylated liposomes to mRNA-PEGylated histidylated polylysine polyplexes; PC: Dipalmitoylphosphatidylcholine; cholesterol, PEG DMG 2000: 1,2-dimyristoyl-sn-glycero-3-phosphoethanolamine-N-[methoxy(polyethylene glycol)-2000]; PS: Phosphatidylserine; Span 85: sorbitane trioleate; unifectin; and squalene. See Martinon F, et al., 1993, Eur. J. Immunol. 23(7), 1719-1722; Hess P R, et al., 2005, Cancer Immunol. Immunother. 55(6), 672-683. Zhou W-Z, et al., 1999. Hum. Gene Ther. 10(16), 2719-2724; Pollard C, et al., 2013, Mol. Ther. 21(1), 251-259; Hoerr I, et al., 2000, Eur. J. Immunol. 30(1), 1-7; Mockey M, et al., 2007, Cancer Gene Ther. 14(9), 802-814; Perche F, et al., 2011, RNA. Nanomed. Nanotechnol. Biol. Med. 7(4), 445-453; Phua KKL, et al., 2014, Sci. Rep. 4, 5128; Geall A J, et al., 2012, Proc. Natl Acad. Sci. USA 109(36), 14604-14609; and Brito L A, et al., 2014, Mol. Ther. 22(12), 2118-2129.

In some embodiments, the lipid nanoparticle comprises a cationic lipid, a PEG-modified lipid, a sterol and a non-cationic lipid. In some embodiments, a cationic lipid is an ionizable cationic lipid and the non-cationic lipid is a neutral lipid, and the sterol is a cholesterol. In some embodiments, a cationic lipid is selected from the group consisting of 2,2-dilinoleyl-4-dimethylaminoethyl-[1,3]-dioxolane (DLin-KC2-DMA), dilinoleyl-methyl-4-dimethylaminobutyrate (DLin-MC3-DMA), di((Z)-non-2-en-1-yl) 9-((4-(dimethylamino)butanoyl)oxy)heptadecanedioate (L319), (12Z,15Z)—N,N-dimethyl-2-nonylhenicosa-12,15-dien-1-amine (L608), and N,N-dimethyl-1-[(1S,2R)-2-octylcyclopropyl]heptadecan-8-amine (L530). In some embodiments, the lipid is (L608).

Lipid nanoparticles, in some embodiments, include one or more ionic lipids, such as non-cationic lipids (e.g., neutral or anionic, or zwitterionic lipids); one or more conjugated lipids (such as PEG-conjugated lipids or lipids conjugated to polymers described in Table 5 of WO2019217941; incorporated herein by reference in its entirety); one or more sterols (e.g., cholesterol).

Lipids that can be used in nanoparticle formations (e.g., lipid nanoparticles) include, for example those described in Table 4 of WO2019217941, which is incorporated by reference—e.g., a lipid-containing nanoparticle can include one or more of the lipids in Table 4 of WO2019217941. Lipid nanoparticles can include additional elements, such as polymers, such as the polymers described in Table 5 of WO2019217941, incorporated by reference.

In some embodiments, conjugated lipids, when present, can include one or more of PEG-diacylglycerol (DAG) (such as 1-(monomethoxy-polyethyleneglycol)-2,3-dimyristoylglycerol (PEG-DMG)), PEG-dialkyloxypropyl (DAA), PEG-phospholipid, PEG-ceramide (Cer), a pegylated phosphatidylethanoloamine (PEG-PE), PEG succinate diacylglycerol (PEGS-DAG) (such as 4-0-(2′,3′-di(tetradecanoyloxy)propyl-1-0-(w-methoxy(polyethoxy)ethyl) butanedioate (PEG-S-DMG)), PEG dialkoxypropylcarbam, N-(carbonyl-methoxypoly ethylene glycol 2000)-1,2-distearoyl-sn-glycero-3-phosphoethanolamine sodium salt, and those described in Table 2 of WO2019051289 (incorporated by reference), and combinations of the foregoing.

In some embodiments, sterols that can be incorporated into lipid nanoparticles include one or more of cholesterol or cholesterol derivatives, such as those in WO2009/127060 or US2010/0130588, which are incorporated by reference. Additional exemplary sterols include phytosterols, including those described in Eygeris et al. (2020), dx.doi.org/10.1021/acs.nanolett.Oc01386, incorporated herein by reference.

In some embodiments, the lipid particle includes an ionizable lipid, a non-cationic lipid, a conjugated lipid that inhibits aggregation of particles, and a sterol. The amounts of these components can be varied independently and to achieve desired properties. For example, in some embodiments, the lipid nanoparticle includes an ionizable lipid is in an amount from about 20 mol % to about 90 mol % of the total lipids (in other embodiments it may be 20-70% (mol), 30-60% (mol) or 40-50% (mol); about 50 mol % to about 90 mol % of the total lipid present in the lipid nanoparticle), a non-cationic lipid in an amount from about 5 mol % to about 30 mol % of the total lipids, a conjugated lipid in an amount from about 0.5 mol % to about 20 mol % of the total lipids, and a sterol in an amount from about 20 mol % to about 50 mol % of the total lipids. The ratio of total lipid to nucleic acid can be varied as desired. For example, the total lipid to nucleic acid (mass or weight) ratio can be from about 10:1 to about 30:1.

In some embodiments, the lipid to nucleic acid ratio (mass/mass ratio; w/w ratio) can be in the range of from about 1:1 to about 25:1, from about 10:1 to about 14:1, from about 3:1 to about 15:1, from about 4:1 to about 10:1, from about 5:1 to about 9:1, or about 6:1 to about 9:1. The amounts of lipids and nucleic acid can be adjusted to provide a desired N/P ratio, for example, N/P ratio of 3, 4, 5, 6, 7, 8, 9, 10 or higher. Generally, the lipid nanoparticle formulation's overall lipid content can range from about 5 mg/ml to about 30 mg/mL.

Some non-limiting example of lipid compounds that may be used (e.g., in combination with other lipid components) to form lipid nanoparticles for the delivery of compositions described herein, e.g., nucleic acid (e.g., RNA or DNA) described herein includes,

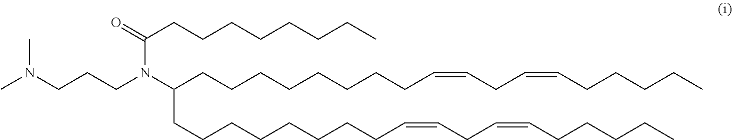 [SMILES: CCCCC/C=C\C/C=C\CCCCCCCCC(CCCCCCCC/C=C\C/C=C\CCCCC)N(CCCN(C)C)C(=O)CCCCCCCC]

In some embodiments an LNP including Formula (i) is used to deliver a polyribonucleotide (e.g., RNA or DNA) composition described herein to cells.

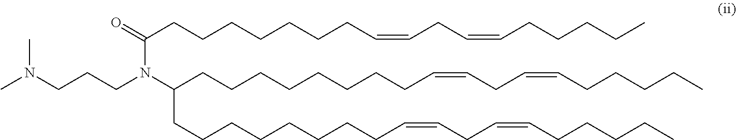 [SMILES: CCCCC/C=C\C/C=C\CCCCCCCCC(CCCCCCCC/C=C\C/C=C\CCCCC)N(CCCN(C)C)C(=O)CCCCCCC/C=C\C/C=C\CCCCC]

In some embodiments an LNP including Formula (ii) is used to deliver a polyribonucleotide (e.g., RNA or DNA) composition described herein to cells.

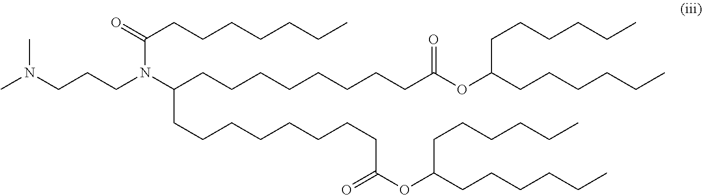 [SMILES: CCCCCCCC(=O)N(CCCN(C)C)C(CCCCCCCCC(=O)OC(CCCCCC)CCCCCC)CCCCCCCCC(=O)OC(CCCCCC)CCCCCC]

In some embodiments an LNP including Formula (iii) is used to deliver a polyribonucleotide (e.g., DNA or RNA) composition described herein to cells.

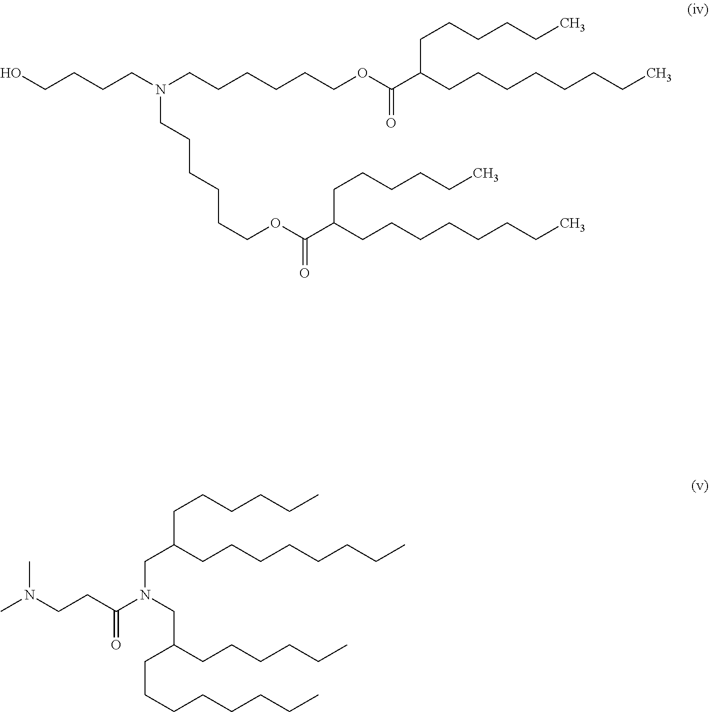

In some embodiments an LNP including Formula (v) is used to deliver a polyribonucleotide (e.g., DNA or RNA) composition described herein to cells.

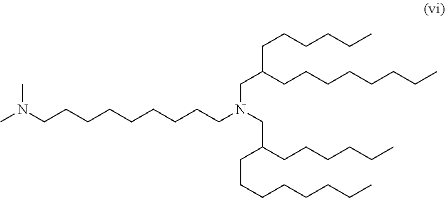 [SMILES: CCCCCCCCC(CCCCCC)CN(CCCCCCCCCN(C)C)CC(CCCCCC)CCCCCCCC]

In some embodiments an LNP including Formula (vi) is used to deliver a polyribonucleotide (e.g., DNA or RNA) composition described herein to cells.

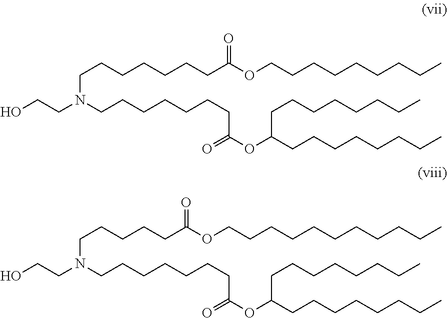 [SMILES: CCCCCCCCCOC(=O)CCCCCCCN(CCO)CCCCCCCC(=O)OC(CCCCCCCC)CCCCCCCC ; CCCCCCCCCCCOC(=O)CCCCCN(CCO)CCCCCCCC(=O)OC(CCCCCCCC)CCCCCCCC]

In some embodiments an LNP including Formula (viii) is used to deliver a polyribonucleotide (e.g., DNA or RNA) composition described herein to cells.

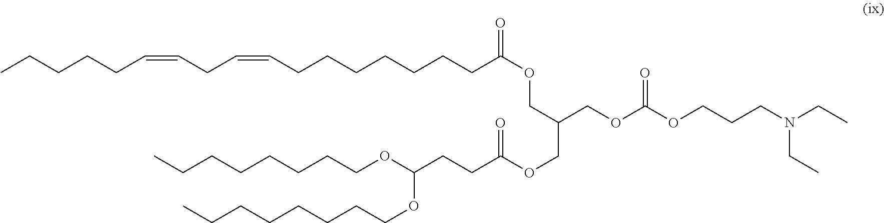 [SMILES: CCCCC/C=C\C/C=C\CCCCCCCC(=O)OCC(COC(=O)CCC(OCCCCCCCC)OCCCCCCCC)COC(=O)OCCCN(CC)CC]

In some embodiments an LNP including Formula (ix) is used to deliver a polyribonucleotide (e.g., DNA or RNA) composition described herein to cells.

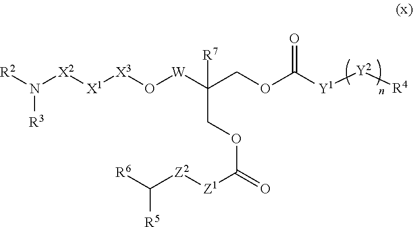

wherein
X¹ is O, NR¹, or a direct bond, X² is C2-5 alkylene, X³ is C(═O) or a direct bond, R¹ is H or Me, R³ is C1-3 alkyl, R² is C1-3 alkyl, or R² taken together with the nitrogen atom to which it is attached and 1-3 carbon atoms of X² form a 4-, 5-, or 6-membered ring, or X¹ is NR¹, R¹ and R² taken together with the nitrogen atoms to which they are attached form a 5- or 6-membered ring, or R² taken together with R³ and the nitrogen atom to which they are attached form a 5-, 6-, or 7-membered ring, Y¹ is C2-12 alkylene, Y² is selected from

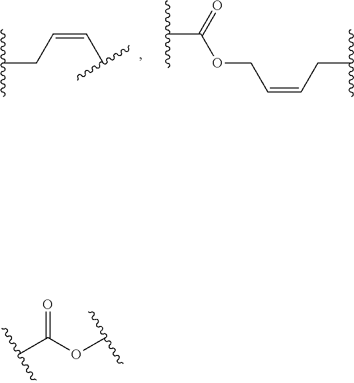 [SMILES: */C=C\C* ; *C/C=C\COC(*)=O ; *OC(*)=O]

(in either orientation), (in either orientation), (in either orientation), n is 0 to 3, R⁴ is C1-15 alkyl, Z¹ is C1-6 alkylene or a direct bond,
Z² is

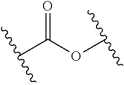 [SMILES: *OC(*)=O]

(in either orientation) or absent, provided that if Z¹ is a direct bond, Z² is absent; R⁵ is C5-9 alkyl or C6-10 alkoxy, R⁶ is C5-9 alkyl or C6-10 alkoxy, W is methylene or a direct bond, and R⁷ is H or Me, or a salt thereof, provided that if R³ and R² are C2 alkyls, X¹ is O, X² is linear C3 alkylene, X³ is C(═O), Y¹ is linear Ce alkylene, (Y²)n-R⁴ is

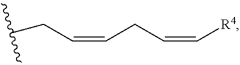

R⁴ is linear C5 alkyl, Z¹ is C2 alkylene, Z² is absent, W is methylene, and R⁷ is H, then R⁵ and R⁶ are not Cx alkoxy.

In some embodiments an LNP including Formula (xii) is used to deliver a polyribonucleotide (e.g., DNA or RNA) composition described herein to cells.

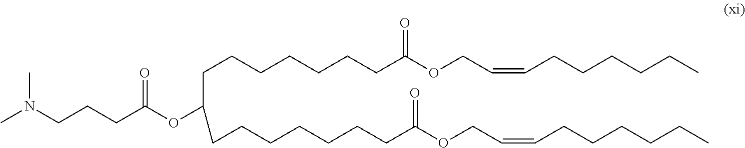 [SMILES: CCCCCC/C=C\COC(=O)CCCCCCCC(CCCCCCCC(=O)OC/C=C\CCCCCC)OC(=O)CCCN(C)C]

In some embodiments an LNP including Formula (xi) is used to deliver a polyribonucleotide (e.g., DNA or RNA) composition described herein to cells.

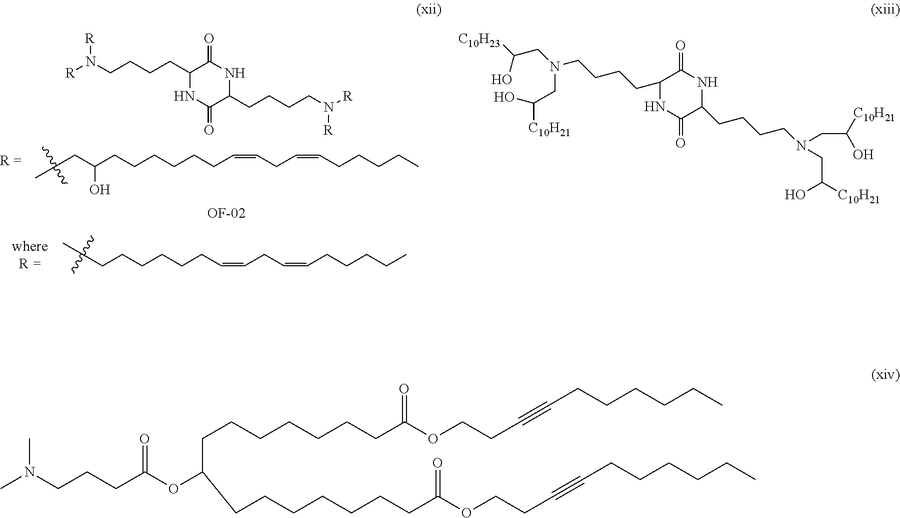

In some embodiments an LNP includes a compound of Formula (xiii) and a compound of Formula (xiv).

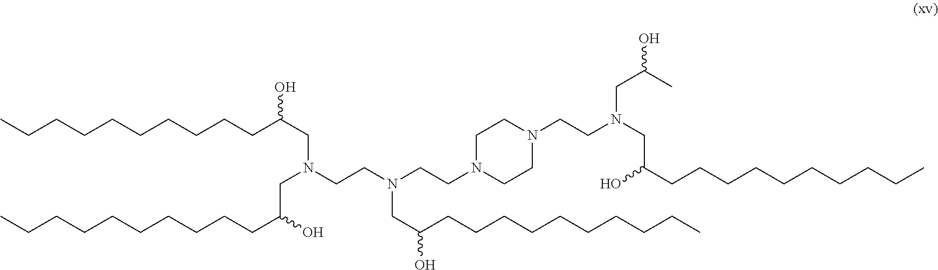 [SMILES: CCCCCCCCCCC(O)CN(CCN1CCN(CCN(CC(C)O)CC(O)CCCCCCCCCC)CC1)CCN(CC(O)CCCCCCCCCC)CC(O)CCCCCCCCCC]

In some embodiments an LNP including Formula (xv) is used to deliver a polyribonucleotide (e.g., DNA or RNA) composition described herein to cells.

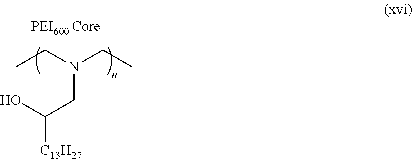

In some embodiments an LNP including a formulation of Formula (xvi) is used to deliver a polyribonucleotide (e.g., DNA or RNA) composition described herein to cells.

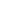

In some embodiments, a lipid compound used to form lipid nanoparticles for the delivery of compositions described herein, e.g., nucleic acid (e.g., RNA or DNA) described herein is made by one of the following reactions:

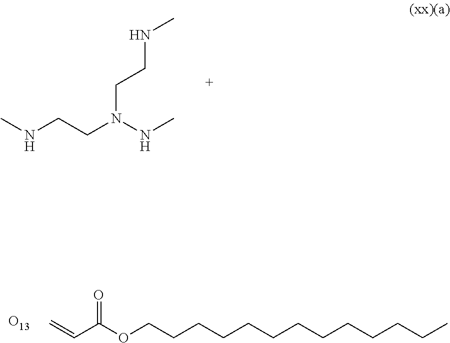

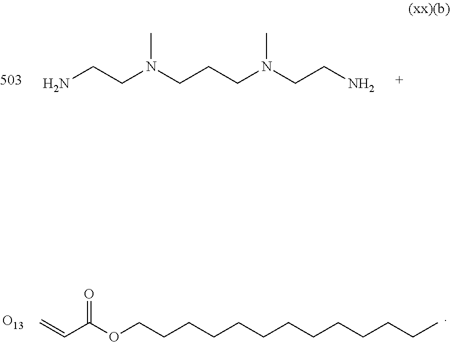 [SMILES: CN(CCN)CCCN(C)CCN ; "O13" ; C=CC(=O)OCCCCCCCCCCCCC ; "."]

In some embodiments an LNP including Formula (xxi) is used to deliver a polyribonucleotide (e.g., DNA or RNA) composition described herein to cells. In some embodiments the LNP of Formula (xxi) is an LNP described by WO2021113777 (e.g., a lipid of Formula (1) such as a lipid of Table 1 of WO2021113777).

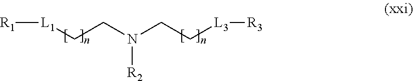

wherein
each n is independently an integer from 2-15; L₁ and L₃ are each independently —OC(O)—* or —C(O)O—*, wherein “*” indicates the attachment point to R₁ or R₃;
R₁ and R₃ are each independently a linear or branched C₉-C₂₀ alkyl or C₉-C₂₀ alkenyl, optionally substituted by one or more substituents selected from a group consisting of oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkynyl, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkyl sulfonyl, and alkyl sulfonealkyl; and
R₂ is selected from a group consisting of:

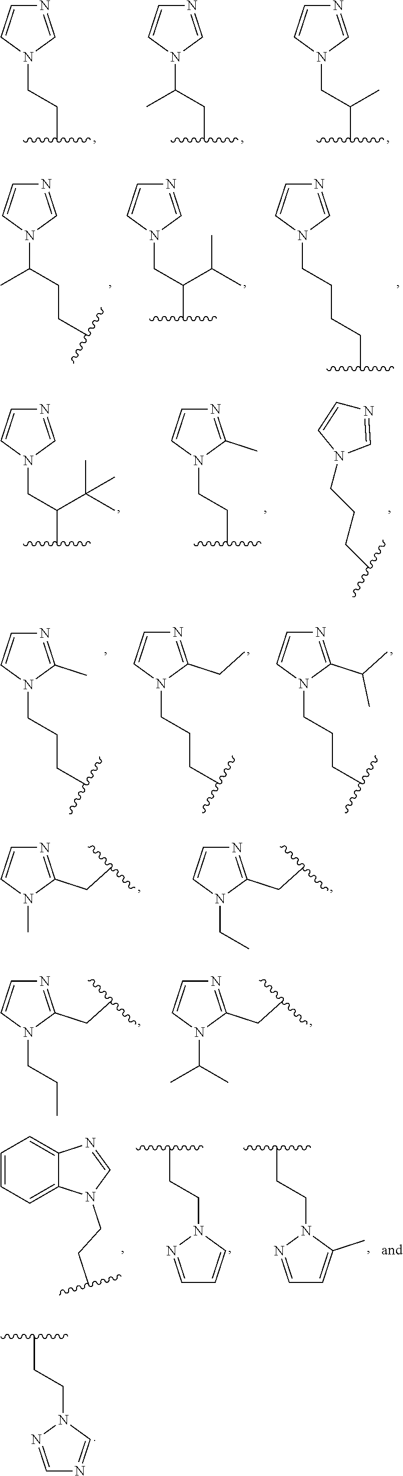

In some embodiments an LNP including Formula (xxii) is used to deliver a polyribonucleotide (e.g., DNA or RNA) composition described herein to cells. In some embodiments the LNP of Formula (xxii) is an LNP described by WO2021113777 (e.g., a lipid of Formula (2) such as a lipid of Table 2 of WO2021113777).

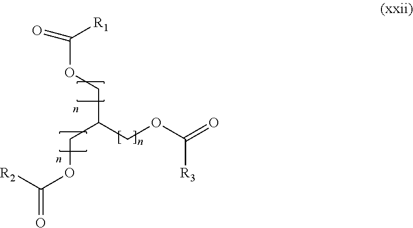

wherein
each n is independently an integer from 1-15;
R₁ and R₂ are each independently selected from a group consisting of

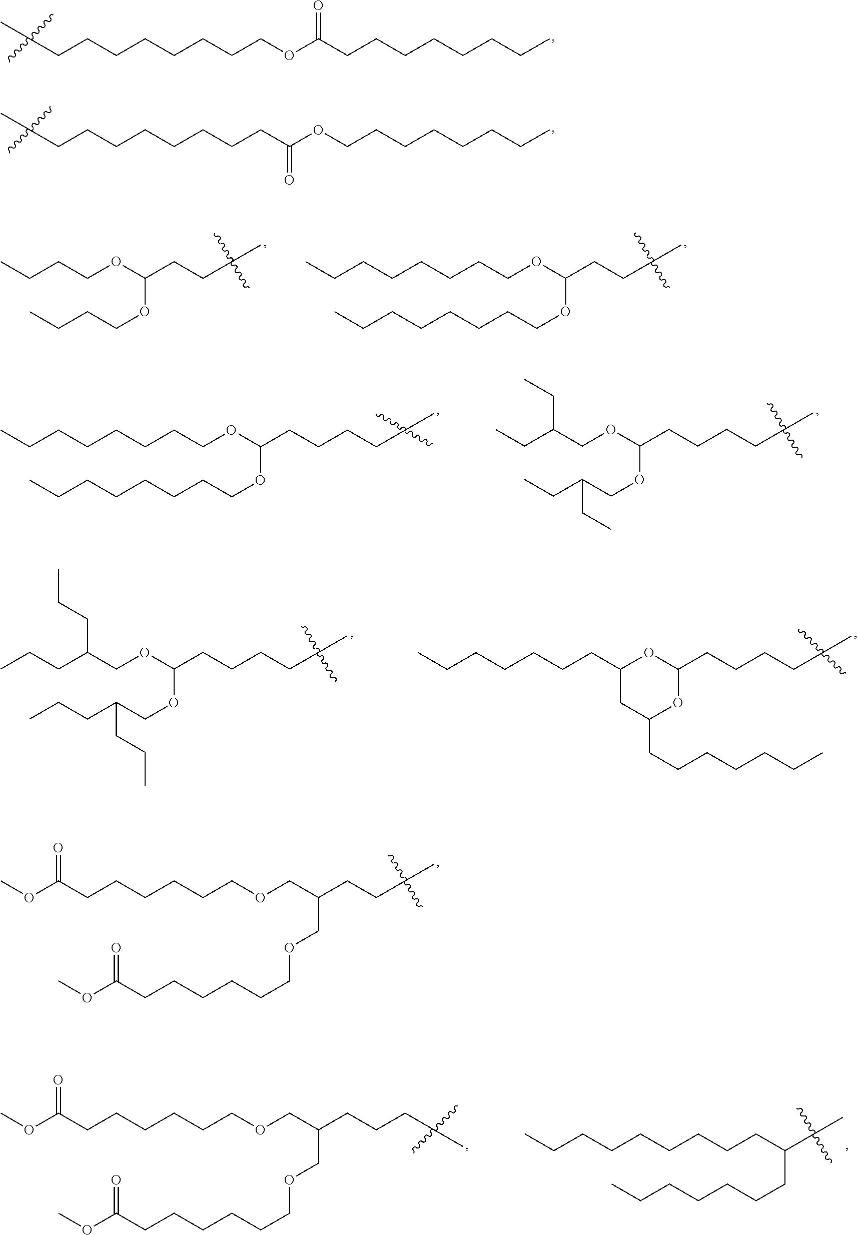

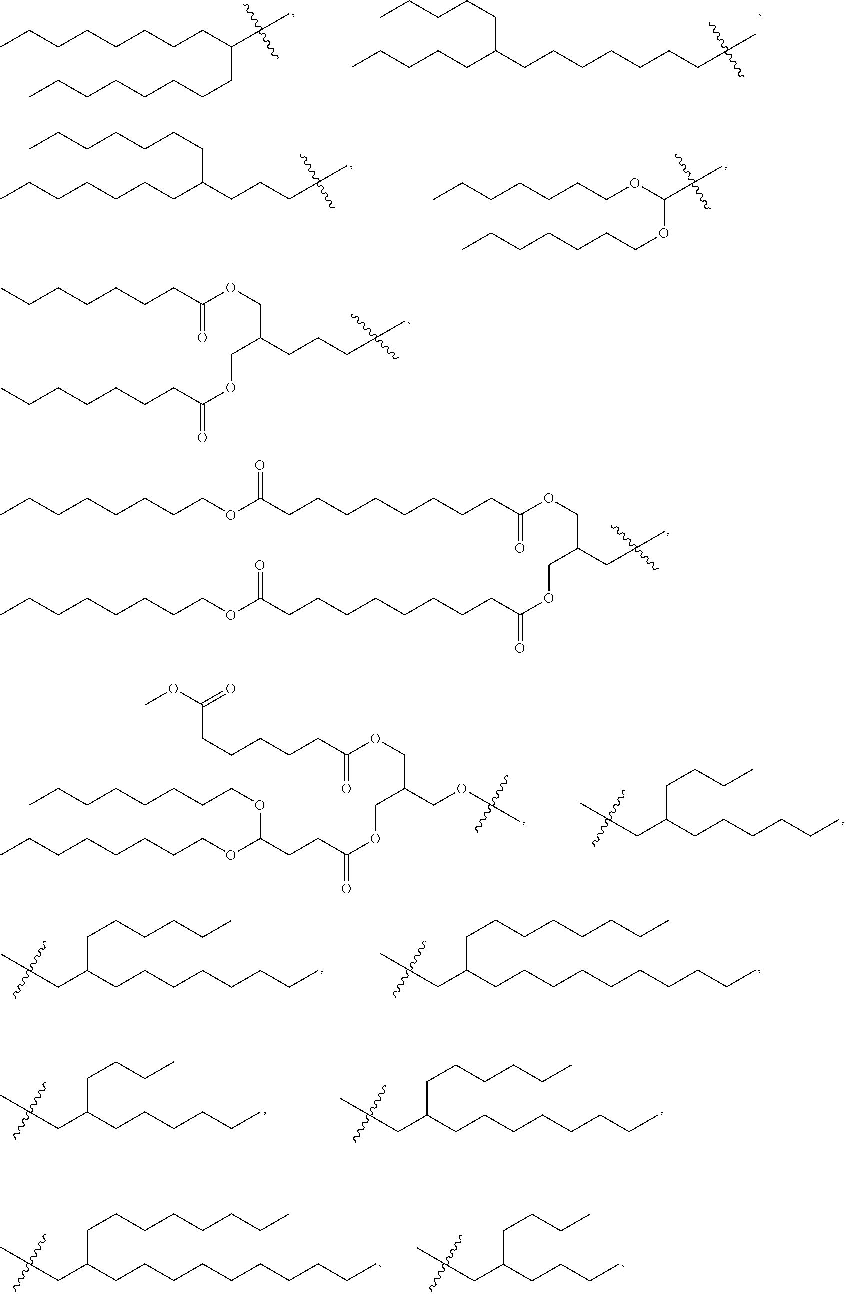

R₃ is selected from a group consisting of:

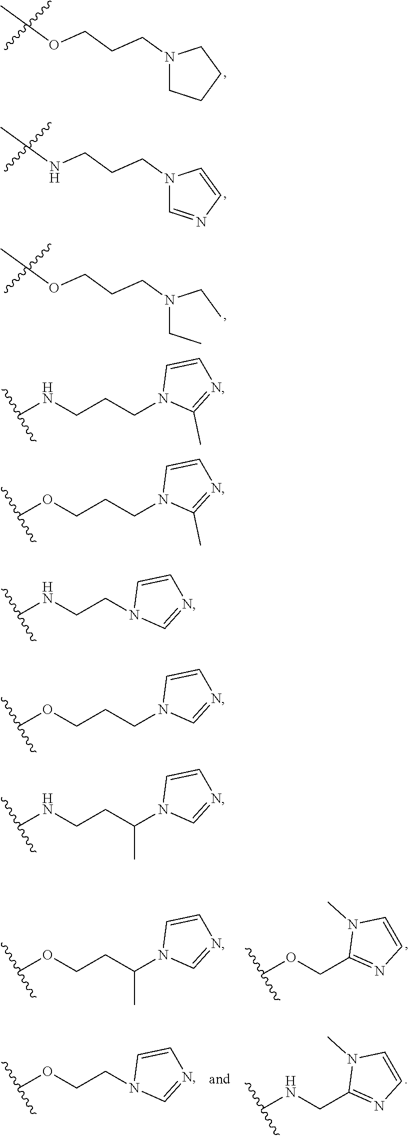

In some embodiments an LNP including Formula (xxiii) is used to deliver a polyribonucleotide (e.g., DNA or RNA) composition described herein to cells. In some embodiments the LNP of Formula (xxiii) is an LNP described by WO2021113777 (e.g., a lipid of Formula (3) such as a lipid of Table 3 of WO2021113777).

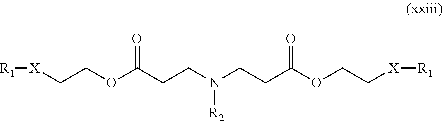

wherein
X is selected from —O—, —S—, or —OC(O)—*, wherein * indicates the attachment point to R₁;
R₁ is selected from a group consisting of:

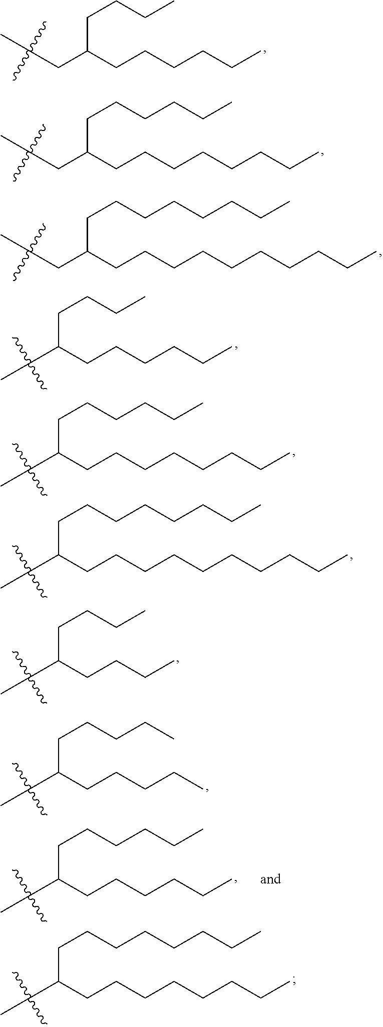

and R₂ is selected from a group consisting of:

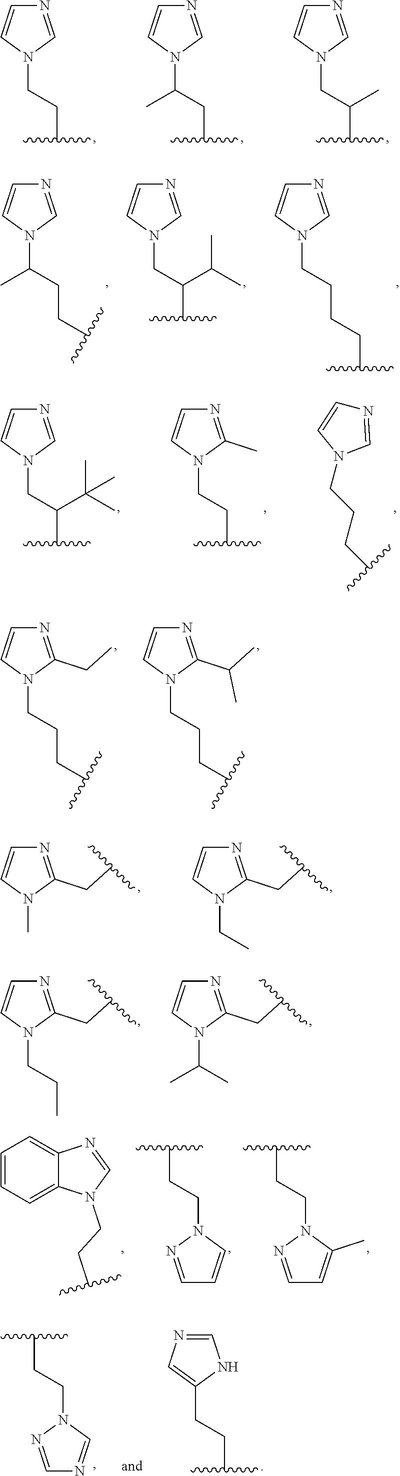

In some embodiments, a composition described herein (e.g. a nucleic acid (e.g., DNA or RNA) or a protein) is provided in an LNP that includes an ionizable lipid. In some embodiments, the ionizable lipid is heptadecan-9-yl 8-((2-hydroxyethyl)(6-oxo-6-(undecyloxy)hexyl)amino)octanoate (SM-102); e.g., as described in Example 1 of U.S. Pat. No. 9,867,888 (incorporated by reference herein in its entirety). In some embodiments, the ionizable lipid is 9Z,12Z)-3-((4,4-bis(octyloxy)butanoyl)oxy)-2-((((3-(diethylamino)propoxy)carbonyl)oxy)methyl)propyl octadeca-9,12-dienoate (LP01), e.g., as synthesized in Example 13 of WO2015/095340 (incorporated by reference herein in its entirety). In some embodiments, the ionizable lipid is Di((Z)-non-2-en-1-yl) 9-((4-dimethylamino)butanoyl)oxy)heptadecanedioate (L319), e.g., as synthesized in Example 7, 8, or 9 of US2012/0027803 (incorporated by reference herein in its entirety). In some embodiments, the ionizable lipid is 1,1′-((2-(4-(2-((2-(Bis(2-hydroxydodecyl)amino)ethyl)(2-hydroxydodecyl)amino)ethyl)piperazin-1-yl)ethyl)azanediyl)bis(dodecan-2-ol) (C12-200), e.g., as synthesized in Examples 14 and 16 of WO2010/053572 (incorporated by reference herein in its entirety). In some embodiments, the ionizable lipid is Imidazole cholesterol ester (ICE) lipid (3S, 10R, 13R, 17R)-10, 13-dimethyl-17-((R)-6-methylheptan-2-yl)-2, 3, 4, 7, 8, 9, 10, 11, 12, 13, 14, 15, 16, 17-tetradecahydro-1H-cyclopenta[a]phenanthren-3-yl 3-(1H-imidazol-4-yl)propanoate, e.g., Structure (I) from WO2020/106946 (incorporated by reference herein in its entirety).

In some embodiments, an ionizable lipid may be a cationic lipid, an ionizable cationic lipid, e.g., a cationic lipid that can exist in a positively charged or neutral form depending on pH, or an amine-containing lipid that can be readily protonated. In some embodiments, the cationic lipid is a lipid capable of being positively charged, e.g., under physiological conditions. Exemplary cationic lipids include one or more amine group(s) which bear the positive charge. In some embodiments, the lipid particle includes a cationic lipid in formulation with one or more of neutral lipids, ionizable amine-containing lipids, biodegradable alkyne lipids, steroids, phospholipids including polyunsaturated lipids, structural lipids (e.g., sterols), PEG, cholesterol, and polymer conjugated lipids. In some embodiments, the cationic lipid may be an ionizable cationic lipid. An exemplary cationic lipid as disclosed herein may have an effective pKa over 6.0. In embodiments, a lipid nanoparticle may include a second cationic lipid having a different effective pKa (e.g., greater than the first effective pKa), than the first cationic lipid. A lipid nanoparticle may include between 40 and 60 mol percent of a cationic lipid, a neutral lipid, a steroid, a polymer conjugated lipid, and a therapeutic agent, e.g., a nucleic acid (e.g., RNA (e.g., DNA or RNA)) described herein, encapsulated within or associated with the lipid nanoparticle. In some embodiments, the nucleic acid is co-formulated with the cationic lipid. The nucleic acid may be adsorbed to the surface of an LNP, e.g., an LNP including a cationic lipid. In some embodiments, the nucleic acid may be encapsulated in an LNP, e.g., an LNP including a cationic lipid. In some embodiments, the lipid nanoparticle may include a targeting moiety, e.g., coated with a targeting agent. In embodiments, the LNP formulation is biodegradable. In some embodiments, a lipid nanoparticle including one or more lipid described herein, e.g., Formula (i), (ii), (ii), (vii) and/or (ix) encapsulates at least 1%, at least 5%, at least 10%, at least 20%, at least 30%, at least 40%, at least 50%, at least 60%, at least 70%, at least 80%, at least 90%, at least 92%, at least 95%, at least 97%, at least 98% or 100% of an RNA molecule.

Exemplary ionizable lipids that can be used in lipid nanoparticle formulations include, without limitation, those listed in Table 1 of WO2019051289, incorporated herein by reference. Additional exemplary lipids include, without limitation, one or more of the following formulae: X of US2016/0311759; I of US20150376115 or in US2016/0376224; I, II or III of US20160151284; I, IA, II, or IIA of US20170210967; I-c of US20150140070; A of US2013/0178541; I of US2013/0303587 or US2013/0123338; I of US2015/0141678; II, III, IV, or V of US2015/0239926; I of US2017/0119904; I or II of WO2017/117528; A of US2012/0149894; A of US2015/0057373; A of WO2013/116126; A of US2013/0090372; A of US2013/0274523; A of US2013/0274504; A of US2013/0053572; A of WO2013/016058; A of WO2012/162210; I of US2008/042973; I, II, III, or IV of US2012/01287670; I or II of US2014/0200257; I, II, or III of US2015/0203446; I or III of US2015/0005363; I, IA, IB, IC, ID, II, IIA, IIB, IIC, IID, or III-XXIV of US2014/0308304; of US2013/0338210; I, II, III, or IV of WO2009/132131; A of US2012/01011478; I or XXXV of US2012/0027796; XIV or XVII of US2012/0058144; of US2013/0323269; I of US2011/0117125; I, II, or III of US2011/0256175; 1, 11, 111, IV, V, VI, VII, VIII, IX, X, XI, XII of US2012/0202871; 1, 11, 111, IV, V, VI, VII, VIII, X, XII, XIII, XIV, XV, or XVI of US2011/0076335; I or II of US2006/008378; I of US2013/0123338; I or X-A-Y-Z of US2015/0064242; XVI, XVII, or XVIII of US2013/0022649; I, II, or III of US2013/0116307; I, II, or III of US2013/0116307; I or II of US2010/0062967; I-X of US2013/0189351; I of US2014/0039032; V of US2018/0028664; I of US2016/0317458; I of US2013/0195920; 5, 6, or 10 of U.S. Pat. No. 10,221,127; III-3 of WO2018/081480; 1-5 or 1-8 of WO2020/081938; 18 or 25 of U.S. Pat. No. 9,867,888; A of US2019/0136231; II of WO2020/219876; 1 of US2012/0027803; OF-02 of US2019/0240349; 23 of U.S. Pat. No. 10,086,013; cKK-E12/A6 of Miao et al (2020); C12-200 of WO2010/053572; 7C1 of Dahlman et al (2017); 304-013 or 503-013 of Whitehead et al; TS-P4C2 of U.S. Pat. No. 9,708,628; I of WO2020/106946; I of WO2020/106946; and (1), (2), (3), or (4) of WO2021/113777. Exemplary lipids further include a lipid of any one of Tables 1-16 of WO2021/113777.

In some embodiments, the ionizable lipid is MC3 (6Z,9Z,28Z,3 lZ)-heptatriaconta-6,9,28,3 l-tetraen-19-yl-4-(dimethylamino) butanoate (DLin-MC3-DMA or MC3), e.g., as described in Example 9 of WO2019051289A9 (incorporated by reference herein in its entirety). In some embodiments, the ionizable lipid is the lipid ATX-002, e.g., as described in Example 10 of WO2019051289A9 (incorporated by reference herein in its entirety). In some embodiments, the ionizable lipid is (13Z,16Z)-A,A-dimethyl-3-nonyldocosa-13, 16-dien-1-amine (Compound 32), e.g., as described in Example 11 of WO2019051289A9 (incorporated by reference herein in its entirety). In some embodiments, the ionizable lipid is Compound 6 or Compound 22, e.g., as described in Example 12 of WO2019051289A9 (incorporated by reference herein in its entirety).

Exemplary non-cationic lipids include, but are not limited to, distearoyl-sn-glycero-phosphoethanolamine, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), monomethyl-phosphatidylethanolamine (such as 16-O-monomethyl PE), dimethyl-phosphatidylethanolamine (such as 16-O-dimethyl PE), 18-1-trans PE, 1-stearoyl-2-oleoyl-phosphatidyethanolamine (SOPE), hydrogenated soy phosphatidylcholine (HSPC), egg phosphatidylcholine (EPC), dioleoylphosphatidylserine (DOPS), sphingomyelin (SM), dimyristoyl phosphatidylcholine (DMPC), dimyristoyl phosphatidylglycerol (DMPG), distearoylphosphatidylglycerol (DSPG), dierucoylphosphatidylcholine (DEPC), palmitoyloleyolphosphatidylglycerol (POPG), dielaidoyl-phosphatidylethanolamine (DEPE), lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidicacid,cerebrosides, dicetylphosphate, lysophosphatidylcholine, dilinoleoylphosphatidylcholine, or mixtures thereof. It is understood that other diacylphosphatidylcholine and diacylphosphatidylethanolamine phospholipids can also be used. The acyl groups in these lipids are preferably acyl groups derived from fatty acids having C10-C24 carbon chains, e.g., lauroyl, myristoyl, paimitoyl, stearoyl, or oleoyl. Additional exemplary lipids, in certain embodiments, include, without limitation, those described in Kim et al. (2020) dx.doi.org/10.1021/acs.nanolett.0c01386, incorporated herein by reference. Such lipids include, in some embodiments, plant lipids found to improve liver transfection with mRNA (e.g., DGTS).

Other examples of non-cationic lipids suitable for use in the lipid nanoparticles include, without limitation, nonphosphorous lipids such as, e.g., stearylamine, dodeeylamine, hexadecylamine, acetyl palmitate, glycerol ricinoleate, hexadecyl stereate, isopropyl myristate, amphoteric acrylic polymers, triethanolamine-lauryl sulfate, alkyl-aryl sulfate polyethyloxylated fatty acid amides, dioctadecyl dimethyl ammonium bromide, ceramide, sphingomyelin, and the like. Other non-cationic lipids are described in WO2017/099823 or US patent publication US2018/0028664, the contents of which is incorporated herein by reference in their entirety.

In some embodiments, the non-cationic lipid is oleic acid or a compound of Formula I, II, or IV of US2018/0028664, incorporated herein by reference in its entirety. The non-cationic lipid can include, for example, 0-30% (mol) of the total lipid present in the lipid nanoparticle. In some embodiments, the non-cationic lipid content is 5-20% (mol) or 10-15% (mol) of the total lipid present in the lipid nanoparticle. In embodiments, the molar ratio of ionizable lipid to the neutral lipid ranges from about 2:1 to about 8:1 (e.g., about 2:1, 3:1, 4:1, 5:1, 6:1, 7:1, or 8:1).

In some embodiments, the lipid nanoparticles do not include any phospholipids.

In some aspects, the lipid nanoparticle can further include a component, such as a sterol, to provide membrane integrity. One exemplary sterol that can be used in the lipid nanoparticle is cholesterol and derivatives thereof. Non-limiting examples of cholesterol derivatives include polar analogues such as 5a-cholestanol, 53-coprostanol, cholesteryl-(2,-hydroxy)-ethyl ether, cholesteryl-(4′-hydroxy)-butyl ether, and 6-ketocholestanol; non-polar analogues such as 5a-cholestane, cholestenone, 5a-cholestanone, 5p-cholestanone, and cholesteryl decanoate; and mixtures thereof. In some embodiments, the cholesterol derivative is a polar analogue, e.g., cholesteryl-(4′-hydroxy)-butyl ether. Exemplary cholesterol derivatives are described in PCT publication WO2009/127060 and US patent publication US2010/0130588, each of which is incorporated herein by reference in its entirety.

In some embodiments, the component providing membrane integrity, such as a sterol, can include 0-50% (mol) (e.g., 0-10%, 10-20%, 20-30%, 30-40%, or 40-50%) of the total lipid present in the lipid nanoparticle. In some embodiments, such a component is 20-50% (mol) 30-40% (mol) of the total lipid content of the lipid nanoparticle.

In some embodiments, the lipid nanoparticle can include a polyethylene glycol (PEG) or a conjugated lipid molecule. Generally, these are used to inhibit aggregation of lipid nanoparticles and/or provide steric stabilization. Exemplary conjugated lipids include, but are not limited to, PEG-lipid conjugates, polyoxazoline (POZ)-lipid conjugates, polyamide-lipid conjugates (such as ATTA-lipid conjugates), cationic-polymer lipid (CPL) conjugates, and mixtures thereof. In some embodiments, the conjugated lipid molecule is a PEG-lipid conjugate, for example, a (methoxy polyethylene glycol)-conjugated lipid.

Exemplary PEG-lipid conjugates include, but are not limited to, PEG-diacylglycerol (DAG) (such as 1-(monomethoxy-polyethyleneglycol)-2,3-dimyristoylglycerol (PEG-DMG)), PEG-dialkyloxypropyl (DAA), PEG-phospholipid, PEG-ceramide (Cer), a pegylated phosphatidylethanoloamine (PEG-PE), PEG succinate diacylglycerol (PEGS-DAG) (such as 4-0-(2′,3′-di(tetradecanoyloxy)propyl-1-0-(w-methoxy(polyethoxy)ethyl) butanedioate (PEG-S-DMG)), PEG dialkoxypropylcarbam, N-(carbonyl-methoxypolyethylene glycol 2000)-1,2-distearoyl-sn-glycero-3-phosphoethanolamine sodium salt, or a mixture thereof. Additional exemplary PEG-lipid conjugates are described, for example, in U.S. Pat. Nos. 5,885,613, 6,287,591, US2003/0077829, US2003/0077829, US2005/0175682, US2008/0020058, US2011/0117125, US2010/0130588, US2016/0376224, US2017/0119904, and US/099823, the contents of all of which are incorporated herein by reference in their entirety. In some embodiments, a PEG-lipid is a compound of Formula III, III-a-I, III-a-2, III-b-1, III-b-2, or V of US2018/0028664, the content of which is incorporated herein by reference in its entirety. In some embodiments, a PEG-lipid is of Formula II of US20150376115 or US2016/0376224, the content of both of which is incorporated herein by reference in its entirety. In some embodiments, the PEG-DAA conjugate can be, for example, PEG-dilauryloxypropyl, PEG-dimyristyloxypropyl, PEG-dipalmityloxypropyl, or PEG-distearyloxypropyl. The PEG-lipid can be one or more of PEG-DMG, PEG-dilaurylglycerol, PEG-dipalmitoylglycerol, PEG-disterylglycerol, PEG-dilaurylglycamide, PEG-dimyristylglycamide, PEG-dipalmitoylglycamide, PEG-disterylglycamide, PEG-cholesterol (1-[8′-(Cholest-5-en-3[beta]-oxy)carboxamido-3′,6′-dioxaoctanyl] carbamoyl-[omega]-methyl-poly(ethylene glycol), PEG-DMB (3,4-Ditetradecoxylbenzyl-[omega]-methyl-poly(ethylene glycol) ether), and 1,2-dimyristoyl-sn-glycero-3-phosphoethanolamine-N-[methoxy(polyethylene glycol)-2000]. In some embodiments, the PEG-lipid includes PEG-DMG, 1,2-dimyristoyl-sn-glycero-3-phosphoethanolamine-N-[methoxy(polyethylene glycol)-2000]. In some embodiments, the PEG-lipid includes a structure selected from:

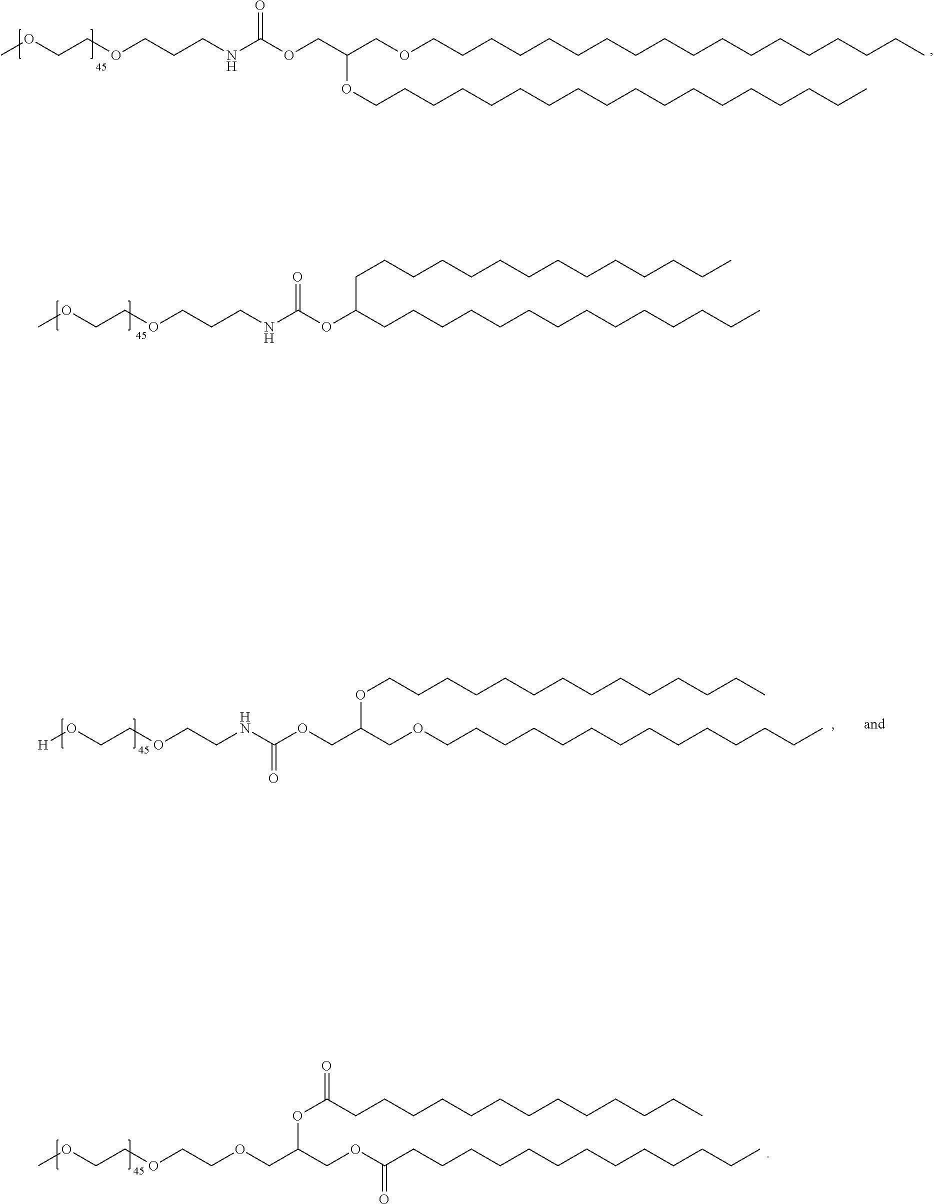

In some embodiments, lipids conjugated with a molecule other than a PEG can also be used in place of PEG-lipid. For example, polyoxazoline (POZ)-lipid conjugates, polyamide-lipid conjugates (such as ATTA-lipid conjugates), and cationic-polymer lipid (GPL) conjugates can be used in place of or in addition to the PEG-lipid.

Exemplary conjugated lipids, i.e., PEG-lipids, (POZ)-lipid conjugates, ATTA-lipid conjugates and cationic polymer-lipids are described in the PCT and LIS patent applications listed in Table 2 of WO2019051289A9, the contents of all of which are incorporated herein by reference in their entirety.

In some embodiments, the PEG or the conjugated lipid can include 0-20% (mol) of the total lipid present in the lipid nanoparticle. In some embodiments, PEG or the conjugated lipid content is 0.5-10% or 2-5% (mol) of the total lipid present in the lipid nanoparticle. Molar ratios of the ionizable lipid, non-cationic-lipid, sterol, and PEG/conjugated lipid can be varied as needed. For example, the lipid particle can include 30-70% ionizable lipid by mole or by total weight of the composition, 0-60% cholesterol by mole or by total weight of the composition, 0-30% non-cationic-lipid by mole or by total weight of the composition and 1-10% conjugated lipid by mole or by total weight of the composition. Preferably, the composition includes 30-40% ionizable lipid by mole or by total weight of the composition, 40-50% cholesterol by mole or by total weight of the composition, and 10-20% non-cationic-lipid by mole or by total weight of the composition. In some other embodiments, the composition is 50-75% ionizable lipid by mole or by total weight of the composition, 20-40% cholesterol by mole or by total weight of the composition, and 5 to 10% non-cationic-lipid, by mole or by total weight of the composition and 1-10% conjugated lipid by mole or by total weight of the composition. The composition may contain 60-70% ionizable lipid by mole or by total weight of the composition, 25-35% cholesterol by mole or by total weight of the composition, and 5-10% non-cationic-lipid by mole or by total weight of the composition. The composition may also contain up to 90% ionizable lipid by mole or by total weight of the composition and 2 to 15% non-cationic lipid by mole or by total weight of the composition. The formulation may also be a lipid nanoparticle formulation, for example including 8-30% ionizable lipid by mole or by total weight of the composition, 5-30% non-cationic lipid by mole or by total weight of the composition, and 0-20% cholesterol by mole or by total weight of the composition; 4-25% ionizable lipid by mole or by total weight of the composition, 4-25% non-cationic lipid by mole or by total weight of the composition, 2 to 25% cholesterol by mole or by total weight of the composition, 10 to 35% conjugate lipid by mole or by total weight of the composition, and 5% cholesterol by mole or by total weight of the composition; or 2-30% ionizable lipid by mole or by total weight of the composition, 2-30% non-cationic lipid by mole or by total weight of the composition, 1 to 15% cholesterol by mole or by total weight of the composition, 2 to 35% conjugate lipid by mole or by total weight of the composition, and 1-20% cholesterol by mole or by total weight of the composition; or even up to 90% ionizable lipid by mole or by total weight of the composition and 2-10% non-cationic lipids by mole or by total weight of the composition, or even 100% cationic lipid by mole or by total weight of the composition. In some embodiments, the lipid particle formulation includes ionizable lipid, phospholipid, cholesterol and a PEG-ylated lipid in a molar ratio of 50:10:38.5:1.5. In some other embodiments, the lipid particle formulation includes ionizable lipid, cholesterol and a PEG-ylated lipid in a molar ratio of 60:38.5:1.5.

In some embodiments, the lipid particle includes ionizable lipid, non-cationic lipid (e.g., phospholipid), a sterol (e.g., cholesterol) and a PEG-ylated lipid, where the molar ratio of lipids ranges from 20 to 70 mole percent for the ionizable lipid, with a target of 40-60, the mole percent of non-cationic lipid ranges from 0 to 30, with a target of 0 to 15, the mole percent of sterol ranges from 20 to 70, with a target of 30 to 50, and the mole percent of PEG-ylated lipid ranges from 1 to 6, with a target of 2 to 5.

In some embodiments, the lipid particle includes ionizable lipid/non-cationic-lipid/sterol/conjugated lipid at a molar ratio of 50:10:38.5:1.5.

In an aspect, the disclosure provides a lipid nanoparticle formulation including phospholipids, lecithin, phosphatidylcholine and phosphatidylethanolamine.

In some embodiments, one or more additional compounds can also be included. Those compounds can be administered separately, or the additional compounds can be included in the lipid nanoparticles of the invention. In other words, the lipid nanoparticles can contain other compounds in addition to the nucleic acid or at least a second nucleic acid, different than the first. Without limitations, other additional compounds can be selected from the group consisting of small or large organic or inorganic molecules, monosaccharides, disaccharides, trisaccharides, oligosaccharides, polysaccharides, peptides, proteins, peptide analogs and derivatives thereof, peptidomimetics, nucleic acids, nucleic acid analogs and derivatives, an extract made from biological materials, or any combinations thereof.

In some embodiments, the LNPs include biodegradable, ionizable lipids. In some embodiments, the LNPs include (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate) or another ionizable lipid. See, e.g., lipids of WO2019/067992, WO/2017/173054, WO2015/095340, and WO2014/136086, as well as references provided therein. In some embodiments, the term cationic and ionizable in the context of LNP lipids is interchangeable, e.g., wherein ionizable lipids are cationic depending on the pH.

In some embodiments, the average LNP diameter of the LNP formulation may be between 10s of nm and 100s of nm, e.g., measured by dynamic light scattering (DLS). In some embodiments, the average LNP diameter of the LNP formulation may be from about 40 nm to about 150 nm, such as about 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm. In some embodiments, the average LNP diameter of the LNP formulation may be from about 50 nm to about 100 nm, from about 50 nm to about 90 nm, from about 50 nm to about 80 nm, from about 50 nm to about 70 nm, from about 50 nm to about 60 nm, from about 60 nm to about 100 nm, from about 60 nm to about 90 nm, from about 60 nm to about 80 nm, from about 60 nm to about 70 nm, from about 70 nm to about 100 nm, from about 70 nm to about 90 nm, from about 70 nm to about 80 nm, from about 80 nm to about 100 nm, from about 80 nm to about 90 nm, or from about 90 nm to about 100 nm. In some embodiments, the average LNP diameter of the LNP formulation may be from about 70 nm to about 100 nm. In a particular embodiment, the average LNP diameter of the LNP formulation may be about 80 nm. In some embodiments, the average LNP diameter of the LNP formulation may be about 100 nm. In some embodiments, the average LNP diameter of the LNP formulation ranges from about 1 mm to about 500 mm, from about 5 mm to about 200 mm, from about 10 mm to about 100 mm, from about 20 mm to about 80 mm, from about 25 mm to about 60 mm, from about 30 mm to about 55 mm, from about 35 mm to about 50 mm, or from about 38 mm to about 42 mm.

A LNP may, in some instances, be relatively homogenous. A polydispersity index may be used to indicate the homogeneity of a LNP, e.g., the particle size distribution of the lipid nanoparticles. A small (e.g., less than 0.3) polydispersity index generally indicates a narrow particle size distribution. A LNP may have a polydispersity index from about 0 to about 0.25, such as 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, or 0.25. In some embodiments, the polydispersity index of a LNP may be from about 0.10 to about 0.20.

The zeta potential of a LNP may be used to indicate the electrokinetic potential of the composition. In some embodiments, the zeta potential may describe the surface charge of an LNP. Lipid nanoparticles with relatively low charges, positive or negative, are generally desirable, as more highly charged species may interact undesirably with cells, tissues, and other elements in the body. In some embodiments, the zeta potential of a LNP may be from about −10 mV to about +20 mV, from about −10 mV to about +15 mV, from about −10 mV to about +10 mV, from about −10 mV to about +5 mV, from about −10 mV to about 0 mV, from about −10 mV to about −5 mV, from about −5 mV to about +20 mV, from about −5 mV to about +15 mV, from about −5 mV to about +10 mV, from about −5 mV to about +5 mV, from about −5 mV to about 0 mV, from about 0 mV to about +20 mV, from about 0 mV to about +15 mV, from about 0 mV to about +10 mV, from about 0 mV to about +5 mV, from about +5 mV to about +20 mV, from about +5 mV to about +15 mV, or from about +5 mV to about +10 mV.

The efficiency of encapsulation of a protein and/or nucleic acid, describes the amount of protein and/or nucleic acid that is encapsulated or otherwise associated with a LNP after preparation, relative to the initial amount provided. The encapsulation efficiency is desirably high (e.g., close to 100%). The encapsulation efficiency may be measured, for example, by comparing the amount of protein or nucleic acid in a solution containing the lipid nanoparticle before and after breaking up the lipid nanoparticle with one or more organic solvents or detergents. An anion exchange resin may be used to measure the amount of free protein or nucleic acid (e.g., RNA) in a solution. Fluorescence may be used to measure the amount of free protein and/or nucleic acid (e.g., RNA) in a solution. For the lipid nanoparticles described herein, the encapsulation efficiency of a protein and/or nucleic acid may be at least 50%, for example 50%, 55%, 60%, 65%, 70%, 75%, 80%, 85%, 90%, 91%, 92%, 93%, 94%, 95%, 96%, 97%, 98%, 99%, or 100%. In some embodiments, the encapsulation efficiency may be at least 80%. In some embodiments, the encapsulation efficiency may be at least 90%. In some embodiments, the encapsulation efficiency may be at least 95%.

A LNP may optionally include one or more coatings. In some embodiments, a LNP may be formulated in a capsule, film, or table having a coating. A capsule, film, or tablet including a composition described herein may have any useful size, tensile strength, hardness, or density.

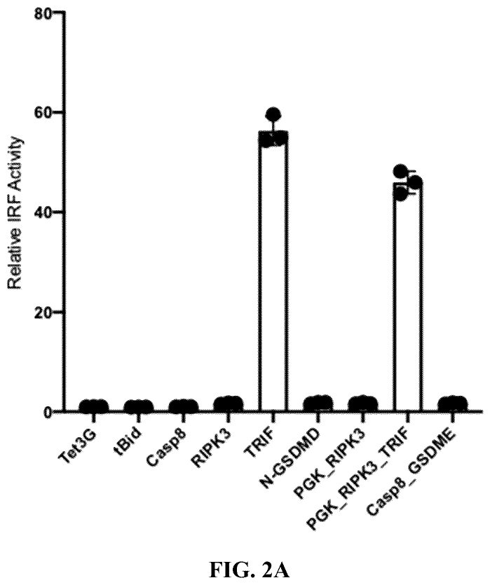

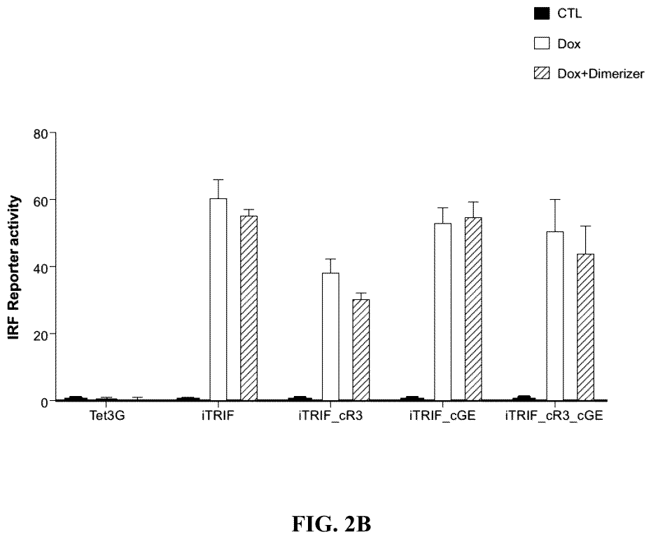

Additional exemplary lipids, formulations, methods, and characterization of LNPs are taught by WO2020/061457 and WO2021/113777, each of which is incorporated herein by reference in its entirety. Further exemplary lipids, formulations, methods, and characterization of LNPs are taught by Hou et al. Lipid nanoparticles for mRNA delivery. Nat Rev Mater (2021). doi.org/10.1038/s41578-021-00358-0, which is incorporated herein by reference in its entirety (see, for example, exemplary lipids and lipid derivatives of FIG. 2 of Hou et al.).

In some embodiments, in vitro or ex vivo cell lipofections are performed using Lipofectamine MessengerMax (Thermo Fisher) or TransIT-mRNA Transfection Reagent (Mirus Bio). In certain embodiments, LNPs are formulated using the GenVoy_ILM ionizable lipid mix (Precision NanoSystems). In certain embodiments, LNPs are formulated using 2,2-dilinoleyl-4-dimethylaminoethyl-[1,3]-dioxolane (DLin-KC2-DMA) or dilinoleylmethyl-4-dimethylaminobutyrate (DLin-MC3-DMA or MC3), the formulation and in vivo use of which are taught in Jayaraman et al. Angew Chem Int Ed Engl 51(34):8529-8533 (2012), incorporated herein by reference in its entirety.

LNP formulations optimized for the delivery of CRISPR-Cas systems, e.g., Cas9-gRNA RNP, gRNA, Cas9 mRNA, are described in WO2019067992 and WO2019067910, both incorporated by reference, and are useful for delivery of DNA or RNA compositions described herein.

Additional specific LNP formulations useful for delivery of nucleic acids (e.g., RNA, DNA) are described in U.S. Pat. Nos. 8,158,601 and 8,168,775, both incorporated by reference, which include formulations used in patisiran, sold under the name ONPATTRO.

Exemplary dosing of polyribonucleotide (e.g., DNA or RNA) LNP may include about 0.1, 0.25, 0.3, 0.5, 1, 2, 3, 4, 5, 6, 8, 10, or 100 mg/kg (RNA). Exemplary dosing of AAV including a polyribonucleotide (e.g., DNA or RNA) may include an MOI of about 10¹¹, 10¹², 10¹³, and 10¹⁴ vg/kg.

Liposomes The RNA molecules may also be formulated using liposomes. Liposomes are artificially prepared vesicles which may primarily be composed of a lipid bilayer and may be used as a delivery vehicle for the administration of nutrients and pharmaceutical formulations. Liposomes can be of different sizes such as, but not limited to, a multilamellar vesicle (MLV) which may be hundreds of nanometers in diameter and may contain a series of concentric bilayers separated by narrow aqueous compartments, a small unicellular vesicle (SUV) which may be smaller than 50 nm in diameter, and a large unilamellar vesicle (LUV) which may be between 50 and 500 nm in diameter. Liposome design may include, but is not limited to, opsonins or ligands in order to improve the attachment of liposomes to unhealthy tissue or to activate events such as, but not limited to, endocytosis. Liposomes may contain a low or a high pH in order to improve the delivery of the pharmaceutical formulations.

The formation of liposomes may depend on the physicochemical characteristics such as, but not limited to, the pharmaceutical formulation entrapped and the liposomal ingredients, the nature of the medium in which the lipid vesicles are dispersed, the effective concentration of the entrapped substance and its potential toxicity, any additional processes involved during the application and/or delivery of the vesicles, the optimization size, polydispersity and the shelf-life of the vesicles for the intended application, and the batch-to-batch reproducibility and possibility of large-scale production of safe and efficient liposomal products.

In some embodiments, pharmaceutical compositions described herein may include, without limitation, liposomes such as those formed from 1,2-dioleyloxy-N,N-dimethylaminopropane (DODMA) liposomes, DiLa2 liposomes from Marina Biotech (Bothell, Wash.), 1,2-dilinoleyloxy-3-dimethylaminopropane (DLin-DMA), 2,2-dilinoleyl-4-(2-dimethylaminoethyl)-[1,3]-dioxolane (DLin-KC2-DMA), and MC3 (US20100324120; herein incorporated by reference in its entirety) and liposomes which may deliver small molecule drugs such as, but not limited to, DOXIL® from Janssen Biotech, Inc. (Horsham, Pa.).

In some embodiments, pharmaceutical compositions described herein may include, without limitation, liposomes such as those formed from the synthesis of stabilized plasmid-lipid particles (SPLP) or stabilized nucleic acid lipid particle (SNALP) that have been previously described and shown to be suitable for oligonucleotide delivery in vitro and in vivo (see Wheeler et al. Gene Therapy. 1999 6:271-281; Zhang et al. Gene Therapy. 1999 6:1438-1447; Jeffs et al. Pharm Res. 2005 22:362-372; Morrissey et al., Nat Biotechnol. 2005 2:1002-1007; Zimmermann et al., Nature. 2006 441:111-114; Heyes et al. J Contr Rel. 2005 107:276-287; Semple et al. Nature Biotech. 2010 28:172-176; Judge et al. J Clin Invest. 2009 119:661-673; deFougerolles Hum Gene Ther. 2008 19:125-132; U.S. Patent Publication No US20130122104; all of which are incorporated herein in their entireties).

In some embodiments, the RNA (e.g., mRNA) molecules may be formulated in a lipid vesicle, which may have crosslinks between functionalized lipid bilayers. In some embodiments, the RNA (e.g., mRNA) molecules may be formulated in a lipid-polycation complex. The formation of the lipid-polycation complex may be accomplished by methods known in the art and/or as described in U.S. Pub. No. 20120178702, herein incorporated by reference in its entirety. As a non-limiting example, the polycation may include a cationic peptide or a polypeptide such as, but not limited to, polylysine, polyornithine and/or polyarginine. In some embodiments, the RNA (e.g., mRNA) molecules may be formulated in a lipid-polycation complex, which may further include a non-cationic lipid such as, but not limited to, cholesterol or dioleoyl phosphatidylethanolamine (DOPE).

In other embodiments, the mRNA molecules may be packaged and delivered in virus-like replicon particles (VRPs) produced by a helper cell line that provides the capsid and glycoprotein genes in trans. In some embodiments, the mRNA molecule is delivered to a subject as free mRNA, i.e. it is not complexed to another molecule. In some embodiments, the mRNA molecule is delivered to a subject as protamine-complexed mRNA. Protamine is a natural cationic nuclear protein expressed in testis. It is a highly specialized molecule that replaces histones during the final condensation of DNA in sperm and is known to stabilize nucleic acids. It has an arginine-rich sequence and spontaneously associates with nucleic acids in vitro. Protamine-complexed mRNA provides both strong gene expression and immunostimulation. See Scheel B et al., 2005, Eur J Immunol. 35:1557-66; Fotin-Mleczek M, 2011, J Immunother. 34:1-15; Fotin-Mleczek M, et al., 2012, J Gene Med. 14:428-39; and Kowalczyk A, et al., 2016, Vaccine 34:3882-93.

### C. Viral Delivery Methods

In certain aspects the disclosure relates to a virus engineered to comprise one or more recombinant nucleic acid molecules encoding IL-12 and or one or more thanotransmission polypeptides as described herein. Any virus that has the capacity to transfer a nucleic acid molecule encoding two or more thanotransmission polypeptides, or IL-12 and one or more thanotransmission polypeptides, into a target cell may be used. For example, in some embodiments, the virus is capable of transporting a heterologous polynucleotide of at least 4, 5, 6, 7, 8, 9 or 10 kb into a target cell. In some embodiments, the virus is capable of transporting a heterologous polynucleotide of between 4-12 kb into a target cell. In some embodiments, the virus is cytolytic, i.e., capable of lysing the target cell. In some embodiments, the virus is oncolytic, i.e., a virus that preferentially infects and/or lyses cancer cells. In some embodiments, the virus preferentially infects the target cell. In some embodiments, the virus preferentially infects rapidly dividing cells (e.g. cancer cells). The virus may be a DNA virus or an RNA virus (e.g. a retrovirus). In some embodiments, the virus is an RNA virus. In some embodiments, the virus is a DNA virus. In some embodiments, the virus is an oncolytic virus. In some embodiments, the oncolytic virus is a DNA virus. In some embodiments, the oncolytic virus is an RNA virus. In some embodiments, the virus is a replicative virus. In some embodiments, the virus is a non-replicative virus. In some embodiments, the DNA virus is a DNA replicative virus, e.g. a DNA replicative oncolytic virus. In some embodiments, the RNA virus is a RNA replicative virus, e.g., a RNA replicative oncolytic virus. In some embodiments, the virus is an anellovirus.

In some embodiments, the virus is a wildtype virus. In some embodiments, the virus is a recombinant virus. In some embodiments, the virus is a pseudotyped virus. The term “pseudotyped virus” as used herein refers to a viral particle formed with a structural and enzymatic core from one virus and the envelope glycoprotein of another. In some embodiments, the pseudotyped virus is replication defective. In some embodiments, the pseudotyped virus is replication competent.

In some embodiments, the virus is capable of reinjecting a host that was previously infected with the virus. This characteristic allows for multiple administrations of the virus to a subject. In some embodiments, the virus innately triggers Z-NA recognition. In a particular embodiment the virus is not an adenovirus or an adeno-associated virus (AAV). In a further particular embodiment, the virus does not comprise a polynucleotide encoding a synthetic multimerization domain, i.e. a non-naturally occurring domain that physically associates with other such domains with sufficient affinity such that the domains are held in proximity to one another.

In some embodiments, it is advantageous for the virus to comprise an inactivating mutation in one or more endogenous viral genes. In some embodiments, the inactivating mutation is in an endogenous viral gene that contributes to virulence of the virus (e.g. ICP34.5), such that the inactivating mutation decreases virulence. In some embodiments, the inactivating mutation is in an endogenous viral gene that restricts turnover of the infected cell (e.g. ICP6 in HSV; E3L in Vaccinia virus), such that the inactivating mutation facilitates or increases turnover of the cell upon infection. In some embodiments, inactivating mutations in viral genes may be combined with expression of additional polynucleotides or polypeptides that modulate virulence or cell turnover. For example, expression of a delta-Zα1 mutant form of Vaccinia virus E3L may be combined with full deletion of ICP34.5 to restore replicative capacity.

Examples of suitable viruses and endogenous viral genes that may be targeted for deactivation are provided in the table below.

TABLE 2
Exemplary viruses and viral genes targeted for mutation.

| Virus | Mutations |
|---|---|
| vesicular stomatitis virus | Native G protein is replaced with a |
| (VSV) | Junin virus G protein |
| respiratory syncytial virus | |
| (RSV) | |
| adenovirus | Adenovirus Early Region 1A (E1A) |
| | Adenovirus Early Region 1B (E1B) |
| herpes simplex virus (HSV) | ICP34.5 is mutated to limit |
| | neurovirulence |
| | ICP47 is mutated to augment antigen |
| | presentation in HSV-1 infected cells |
| | ICP6 mutation of the RHIM domain (e.g. |
| | a four amino acid change) |
| | mutations at the C-terminus of ICP6 |
| | that inhibit Caspase-8 binding |
| Vaccinia virus | Mutate the Za domain of E3L to prevent |
| | Zα-nucleic acid recognition by the |
| | innate immune system |

In some embodiments, the virus engineered to comprise one or more polynucleotides that promote thanotransmission is selected from the group consisting of adenovirus, herpes simplex virus (HSV), poxyvirus (e.g., Vaccinia virus), a respiratory syncytial virus (RSV), adeno-associated virus (AAV), Coxsackievirus, Newcastle disease virus, Measles Virus, Myxomatosis, Poliovirus, Lentivirus, Vesicular Stomatitis Virus, a retrovirus, foamy virus, farmington virus, Parvoviruses, and influenza virus. In some embodiments, the virus engineered to comprise one or more polynucleotides that promote thanotransmission is selected from the group consisting of a Vaccinia virus, a herpes simplex virus (HSV) and a respiratory syncytial virus (RSV).

In some embodiments, the virus engineered to comprise one or more recombinant nucleic acid molecues that encode a thanotransmission polypeptide is a vesicular stomatitis virus (VSV). Vesicular stomatitis virus is a non-segmented, negative-stranded RNA virus that belongs to the family Rhabdoviridae, genus Vesiculovirus. VSV infects a broad range of animals, including cattle, horses, and swine. The genome of the virus encodes five major proteins, glycoprotein (G), matrix protein (M), nucleoprotein (N), large protein (L), and phosphoprotein (P). The G protein mediates both viral binding and host cell fusion with the endosomal membrane following endocytosis. The L and P proteins are subunits of the viral RNA-dependent RNA polymerase. Previous studies demonstrated that VSV forms a pseudotype when a cell is co-infected with VSV and other enveloped viruses. Pseudotype VSVs in which VSV G proteins are completely replaced with other types of viral envelope proteins, have been established. See Tani et al., 2012, Microbiol. 18: Article 272, pp. 1-7, which is incorporated by reference herein in its entirety.

In some embodiments, the VSV is the wildtype Indiana strain of VSV. In some embodiments, the VSV is a pseudotyped virus with a modified G protein, e.g. a G protein from a different virus. In some embodiments, the VSV is the Indiana strain of VSV comprising a Junin virus G protein.

In some embodiments, the virus engineered to comprise one or more recombinant nucleic acid molecules that encode a thanotransmission polypeptide is an adenovirus. In some embodiments, the adenovirus is adenovirus serotype 5 (Ad5). In some embodiments, the adenovirus is adenovirus serotype 19A (Ad19A). In some embodiments, the adenovirus is adenovirus serotype 26 (Ad26). An adenovirus of one serotype may be engineered to comprise a fiber protein from a different adenovirus serotype. For example, in some embodiments, Ad5 is engineered to substitute the fiber protein from adenovirus serotype 35 (Ad35). This chimeric virus is referred to as Ad5/F35. (See Yotnda et al., 2001, Gene Therapy 8: 930-937, which is incorporated by reference herein in its entirety.) In some embodiments, Ad5 is engineered to substitute the fiber protein from adenovirus serotype 3 (Ad3). This chimeric virus is referred to as Ad5/F3.

In some embodiments, the adenovirus comprises one or more mutations (e.g., one or more substitutions, additions or deletions) relative to a corresponding wildtype adenovirus. For example, in some embodiments, the adenovirus (e.g., Ad5 or Ad5/F35) comprises a deletion in the Adenovirus Early Region 1A (E1A). In some embodiments, the adenovirus (e.g., Ad5 or Ad5/F35) comprises a 24 bp deletion in E1A. This deletion makes viral replication specific to cells with an altered Rb pathway. In some embodiments, the adenovirus (e.g., Ad5 or Ad5/F35) comprises a deletion in the Adenovirus Early Region 1B (E1B). In some embodiments, the adenovirus (e.g., Ad5 or Ad5/F35) comprises a 827 bp deletion in E1B. This deletion allows the virus to replicate in cells with P53 alterations. In a particular embodiment, the adenovirus (e.g., Ad5 or Ad5/F35) comprises a 24 bp deletion in E1A and a 827 bp deletion in E1B. In some embodiments, the adenovirus (e.g., Ad5 or Ad5/F35) has an Arg-Gly-Asp (RGD)-motif engineered into the fiber-H loop. This modification makes the adenovirus use αvβ and αvβ5 integrins (which are expressed in cancer cells) to enter the cell. (See Reynolds et al., 1999, Gene Therapy 6: 1336-1339, which is incorporated by reference herein in its entirety.)

In some embodiments, the adenovirus contains a modified or mutated fiber region. The modified or mutated fiber region may enhance or alter virus tropism and receptor binding.

In some embodiments a polynucleotide as described herein (e.g., a polynucleotide encoding a thanotransmission polypeptide) may be inserted into the E1 region of the adenovirus, e.g. in E1A or E1B. For example, in some embodiments the E1 region is removed and replaced with the polynucleotide. The polynucleotide may be operably linked to a promoter as described herein, e.g., a promoter that is heterologous to the virus. In some embodiments, a polynucleotide as described herein (e.g., a polynucleotide encoding a thanotransmission polypeptide) may be inserted downstream of an endogenous viral promoter to drive expression of the polynucleotide. For example, in some embodiments, the polynucleotide is inserted into an adenovirus downstream of the adenovirus major late promoter, which drives L5 protein expression. The adenovirus major late promoter confers expression concomitant with late viral gene expression. In some embodiments, the polynucleotide is inserted downstream of the endogenous viral gene encoding the L5 protein. In some embodiments, expression of the polynucleotide is linked to L5 expression using a 2A linker disposed between the polynucleotide and the gene encoding the L5 protein. In some embodiments, expression of the polynucleotide is linked to L5 expression by preceding the polynucleotide with an adenoviral splice acceptor under the control of the adenovirus major late promoter.

In some embodiments, the virus engineered to comprise one or more polynucleotides that promote thanotransmission is a herpes simplex virus (HSV), e.g. HSV1. In some embodiments, the HSV1 is selected from Kos, F1, MacIntyre, McKrae and related strains. The HSV may be defective in one or more genes selected from ICP6, ICP34.5, ICP47, UL24, UL55, and UL56. In a particular embodiment, the ICP34.5 encoding gene is replaced by a polynucleotide cassette comprising a US11 encoding gene operably linked to an immediate early (IE) promoter. In a further particular embodiment, the HSV comprises a ΔZα mutant form of a Vaccinia virus E3L gene.

In one embodiment, the HSV is defective in one or more functions of ICP6. For example, mutation of the ICP6 gene may result in different losses of function depending on the mutation. In some embodiments, the ICP6 comprises one or more mutations of the receptor-interacting protein homotypic interaction motif (RHIM) domain. In some embodiments, the ICP6 comprises one or more mutations at the C-terminus that inhibit caspase-8 binding. In some embodiments, the ICP6 comprises one or more mutations that reduces or eliminates ribonucleotide reductase (RR) activity.

In some embodiments, the HSV expresses the US11 gene as an immediate early gene. The US11 protein is required for protein translation regulation late in the viral life cycle. Immediate-early expression of US11 is able to compensate for a loss-of-function mutation in ICP34.5 and so to counteract the shutoff of protein synthesis in a mutant virus with a deletion of ICP34.5, resulting in a less attenuated virus.

In other embodiments, the virus belongs to the Poxviridae family, e.g. a virus selected from myxoma virus, Yaba-like disease virus, raccoonpox virus, orf virus and cowpox virus. In some embodiments, the virus belongs to the Chordopoxvirinae subfamily of the Poxviridae family. In some embodiments, the virus belongs to the Orthopoxvirus genus of the Chordopoxvirinae subfamily. In some embodiments, the virus belongs to the Vaccinia virus species of the Orthopoxvirus genus. In some embodiments, the Vaccinia virus is a strain selected from the group consisting of Dairenl, IHD-J, L-IPV, LC16M8, LC16MO, Lister, LIVP, Tashkent, WR 65-16, Wyeth, Ankara, Copenhagen, Tian Tan and WR.

In one embodiment, the Vaccinia virus is engineered to lack thymidine kinase (TK) activity. In one embodiment, the Vaccinia virus has an inactivating mutation or deletion in the J2R gene that reduces or eliminates TK activity. The J2R gene encodes a TK that forms part of the salvage pathway for pyrimidine deoxyribonucleotide synthesis. In some embodiments, the Vaccinia virus is engineered to lack ribonucleotide reductase (RR) activity. In some embodiments, the Vaccinia virus has an inactivating mutation or deletion in a gene selected from I4L and F4L gene that reduces or eliminates RR activity. Reductions in TK activity or RR activity increases replication of the virus in transformed cells (e.g. cancer cells).

Vaccinia virus encodes multiple proteins that interfere with apoptotic, necroptotic and pyroptotic signaling. For example, E3, which is encoded by the E3L gene, is an important interferon antagonist that also affects Vaccinia host range and contributes to virulence. E3 was characterized first as a 25-kDa dsRNA binding protein that antagonizes the anti-viral activity of the interferon-induced dsRNA binding protein PKR and possesses a C-terminal dsRNA binding domain. The N-terminal region of E3 forms a distinct domain that has similarity with Z-DNA binding proteins and both N- and C-terminal domains contribute to virus virulence. E3 was also described as an apoptosis inhibitor when HeLa cells infected with a mutant Vaccinia lacking the E3L gene resulted in rapid cell death. See Veyer et al., 2017, Immunology Letters 186: 68-80. Accordingly, in some embodiments, the Vaccinia virus is defective in the E3L gene. In some embodiments, the E3L gene has a mutation that results in induction of necroptosis upon infection of a cancer cell.

In some embodiments, the virus (e.g. HSV) comprises a microRNA (miR) target sequence. The miR target sequence prevents viral pathogenesis in normal cells without impeding virus replication in tumor cells. The miR target sequence may be inserted into one or more viral gene loci, e.g. one or more viral genes required for replication of the virus in normal (e.g. non-cancerous) cells. An exemplary microRNA target sequence for inclusion in the virus is miR-124, which has particular application for neural applications. Other microRNA target sequences can alternatively be employed for protecting other types of tissues, and it is within the ordinary skill in the art to select a suitable microRNA target sequence to protect a desired tissue or cell type. For example, miR-122 and miR-199 are expressed in normal liver cells but not primary liver cancer; thus one or a combination of miR-122 and/or miR-199 microRNA target sequences can be employed in embodiments of the viruses for treatment of liver cancers. Similarly, target sequences for miR-128 and/or miR-137 microRNA can be employed in the virus for protection of normal brain. An exemplary microRNA target sequence can be the reverse complement of the microRNA.

In some embodiments, the microRNA target sequences are included in the 3′ untranslated region (“UTR) of an HSV gene, to silence that gene in the presence of the microRNA. Multiple copies (e.g. two copies, three copies, four copies, five copies, six copies, or more) of the microRNA target sequence may be inserted in tandem. The multiple copies of the micro-RNA target sequence may be separated by spacers of four or more nucleotides (e.g. eight or more nucleotides). Without wishing to be bound by theory, it is believed that greater spacing (e.g., larger than about 8 nucleotides) provides increased stability.

To assist in protecting non-cancerous cells from the lytic effect of HSV infection, the multiple copies of the microRNA target sequence are inserted in the 3′ UTR of an HSV gene that is essential for replication in non-cancerous cells, which are known to persons of ordinary skill. The site may be the 3′ UTR of the microRNA-targeted gene in its normal (or native) locus within the HSV genome. In a particular embodiment, the virus is an HSV that includes multiple copies of the microRNA target sequence inserted into the 3′UTR of the ICP4 gene, e.g. one or both copies of the ICP4 gene, in viruses that have both native copies of the ICP4 gene.

In certain embodiments, the genome of the virus contains a deletion of the internal repeat (joint) region comprising one copy each of the diploid genes ICP0, ICP34.5, LAT and ICP4 along with the promoter for the ICP47 gene. In other embodiments, instead of deleting the joint, the expression of genes in the joint region, particularly ICP0 and/or ICP47, can be silenced by deleting these genes or otherwise limited mutagenesis of them.

In some embodiments, the virus comprises a ligand specific for a molecule (e.g. a protein, lipid or carbohydrate) present on the surface of a target cell, e.g. a cancer cell. The ligand may be incorporated into a glycoprotein exposed on the viral surface (e.g. gD or gC of HSV) to facilitate targeting the desired cell with the ligand. For example, the ligand can be incorporated between residues 1 and 25 of gD. Exemplary ligands for targeting GBM and other cancer cells include those targeting EGFR and EGFRVIII, CD133, CXCR4, carcinoembryonic antigen (CEA), ClC-3/annexin-2/MMP-2, human transferrin receptor and EpCAM. The ligand may target such a receptor or cell-surface molecule, i.e., the ligand can be capable of specifically binding such receptor or cell-surface molecule. EGFR- and EGFRVIII-specific ligands, such as antibodies (e.g. single chain antibodies) and VHHs (single domain antibodies), have been described in the literature (Kuan et al. Int. J. Cancer, 88,962-69 (2000); Wickstrand et al., Cancer Res., 55(14):3140-8 (1995); Omid far et al., Tumor Biology, 25:296-305 (2004); see also Uchidaetal. Molecular Therapy, 21:561-9 (2013); see also Braidwood et al., Gene Then, 15, 1579-92 (2008)).

The virus also or alternatively may be targeted by incorporating ligands into other cell-surface molecules or receptors that are not necessarily cancer-associated. For example, ligands can include binding domains from natural ligands (e.g., growth factors (such as EGF, which can target EGFR, NGF, which can target trkA and the like)), peptide or non-peptide hormones, peptides selecting for binding a target molecule (e.g., designed ankyrin repeat proteins (DARPins)), etc. The virus also can include a mutant form of gB and/or gD that facilitates vector entry though non-canonical receptors (and may also have such mutations in one or both of these genes within the HSV genome).

The virus comprising the recombinant nucleic acid molecule encoding one or more thanotransmission polypeptides may further comprise a polynucleotide encoding a matrix metalloproteinase, e.g. matrix metalloproteinase 9 (“MMP9), which degrades collagen type IV, a major component of the of the extracellular matrix (ECM) and basement membranes of glioblastomas (Mammato et al., Am. J. Pathol., 183(4): 1293-1305 (2013), doi: 10.1016/j.ajpath.2013.06.026. Epub 2013 Aug. 5). Expression of a matrix metalloproteinase by the engineered virus enhances infection of tumor cells by the virus due to lateral spread and enhancing tumor-killing activity. Polynucleotides encoding other genes that enhance lateral spread of the virus may also be used.

The recombinant nucleic acid molecules encoding one or more thanotransmission polypeptides as described herein may be comprised within a virus comprising mutations in viral genes. For example, in a particular embodiment, the virus is HSV1 comprising an inactivating mutation (e.g., a deletion) in the ICP34.5 and ICP47 genes, an inactivating mutation in the RHIM domain of ICP6, and polynucleotides encoding ZBP1, RIPK3 and vMLKL. In a further particular embodiment, the virus is HSV1 comprising an inactivating mutation (e.g. a deletion) of ICP47, a replacement of ICP34.5 with a delta-Zα1 mutant form of the Vaccinia virus E3L gene, and polynucleotides encoding ZBP1, RIPK3 and vMLKL. In a further particular embodiment, the virus is a Vaccinia virus comprising a mutation in the Zα1 domain of the E3L gene, and polynucleotides encoding ZBP1, RIPK3 and vMLKL.

### D. Polypeptide Delivery Methods

In some embodiments, IL-12 and/or one or more thanotransmission polypeptides as described herein may be administered directly to a subject. For example, in certain aspects, the disclosure relates to a pharmaceutical composition comprising IL-12 and/or one or more thanotransmission polypeptides selected from the group consisting of RIF, RIPK3, Gasdermin E, vICA, Npro, A238L, vMLKL, caspase 1, and variants (e.g., functional fragments) thereof, or a dominant negative variant of IKBa; and a pharmaceutically acceptable carrier.

In certain aspects, the disclosure relates to a method of promoting thanotransmission in a subject, the method comprising administering a pharmaceutical composition comprising IL-12 and/or one or more different thanotransmission polypeptides as described herein to the subject in an amount and for a time sufficient to promote thanotransmission.

In certain aspects, the disclosure relates to a method of increasing immune response in a subject in need thereof, the method comprising administering a pharmaceutical composition comprising IL-12 and/or one or more thanotransmission polypeptides as described herein to the subject in an amount and for a time sufficient to increase immune response in the subject.

In certain aspects, the disclosure relates to a method of treating a cancer in a subject in need thereof, the method comprising administering a pharmaceutical composition comprising IL-12 and/or one or more thanotransmission polypeptides as described herein to the subject in an amount and for a time sufficient to treat the cancer.

### E. Other Carriers

The compositions, methods, and delivery systems (e.g., DNA, RNA, virus and polypeptide delivery systems) provided by the present disclosure may employ any suitable carrier. General considerations for carriers and delivery of pharmaceutical agents may be found, for example, in Delivery Technologies for Biopharmaceuticals: Peptides, Proteins, Nucleic Acids and Vaccines (Lene Jorgensen and Hanne Morck Nielson, Eds.) Wiley; 1st edition (Dec. 21, 2009); and Vargason et al. 2021. Nat Biomed Eng 5, 951-967.

Non-limiting examples of carriers include carbohydrate carriers (e.g., an anhydride-modified phytoglycogen or glycogen-type material, GalNAc), nanoparticles (e.g., a nanoparticle that encapsulates or is covalently linked to the construct, gold nanoparticles, silica nanoparticles), lipid particles (e.g., liposomes, lipid nanoparticles), cationic carriers (e.g., a cationic lipopolymer or transfection reagent), fusosomes, non-nucleated cells (e.g., ex vivo differentiated reticulocytes), nucleated cells, exosomes, protein carriers (e.g., a protein covalently linked to the construct), peptides (e.g., cell-penetrating peptides), materials (e.g., graphene oxide), single pure lipids (e.g., cholesterol), DNA origami (e.g., DNA tetrahedron).

In one embodiment, the compositions, constructs and systems described herein can be formulated in liposomes or other similar vesicles. Liposomes are spherical vesicle structures composed of a uni- or multilamellar lipid bilayer surrounding internal aqueous compartments and a relatively impermeable outer lipophilic phospholipid bilayer. Liposomes may be anionic, neutral or cationic. Liposomes are biocompatible, nontoxic, can deliver both hydrophilic and lipophilic drug molecules, protect their cargo from degradation by plasma enzymes, and transport their load across biological membranes and the blood brain barrier (BBB) (see, e.g., Spuch and Navarro, Journal of Drug Delivery, vol. 2011, Article ID 469679, 12 pages, 2011. doi:10.1155/2011/469679 for review).

Vesicles can be made from several different types of lipids; however, phospholipids are most commonly used to generate liposomes as drug carriers. Methods for preparation of multilamellar vesicle lipids are known in the art (see for example U.S. Pat. No. 6,693,086, the teachings of which relating to multilamellar vesicle lipid preparation are incorporated herein by reference). Although vesicle formation can be spontaneous when a lipid film is mixed with an aqueous solution, it can also be expedited by applying force in the form of shaking by using a homogenizer, sonicator, or an extrusion apparatus (see, e.g., Spuch and Navarro, Journal of Drug Delivery, vol. 2011, Article ID 469679, 12 pages, 2011. doi:10.1155/2011/469679 for review). Extruded lipids can be prepared by extruding through filters of decreasing size, as described in Templeton et al., Nature Biotech, 15:647-652, 1997, the teachings of which relating to extruded lipid preparation are incorporated herein by reference.

Exosomes can also be used as drug delivery vehicles for the compositions and systems described herein. For a review, see Ha et al. July 2016. Acta Pharmaceutica Sinica B. Volume 6, Issue 4, Pages 287-296; https://doi.org/10.1016/j.apsb.2016.02.001.

Ex vivo differentiated red blood cells can also be used as a carrier for an agent described herein. See, e.g., WO2015073587; WO2017123646; WO2017123644; WO2018102740; wO2016183482; WO2015153102; WO2018151829; WO2018009838; Shi et al. 2014. Proc Natl Acad Sci USA. 111(28): 10131-10136; U.S. Pat. No. 9,644,180; Huang et al. 2017. Nature Communications 8: 423; Shi et al. 2014. Proc Natl Acad Sci USA. 111(28): 10131-10136.

Fusosome compositions, e.g., as described in WO2018208728, can also be used as carriers to deliver the compositions and constructs described herein.

In some embodiments, the carrier is a lipid nanoparticle as described herein.

### VI. Methods of Promoting Thanotransmission

In certain aspects, the disclosure relates to a method of promoting thanotransmission in a subject, the method comprising administering a recombinant nucleic acid molecule, vector, cell, or pharmaceutical composition as described herein to the subject in an amount and for a time sufficient to promote thanotransmission. For example, expression of the thanotransmission polypeptides induces the target cell to produce factors that are actively released by the target cell or become exposed during turnover (e.g. death) of the target cell. These factors signal a responding cell (e.g., an immune cell) to undergo a biological response (e.g. an increase in immune activity).

A. Methods of Increasing Immune Activity

In some aspects, the thanotransmission polypeptides described herein may be used to increase immune activity in a subject, for example, a subject who would benefit from increased immune activity. In certain aspects, the disclosure relates to a method of increasing immune response in a subject in need thereof, the method comprising administering any one of the recombinant nucleic acid molecules, vectors, cells, or pharmaceutical compositions described herein to the subject in an amount and for a time sufficient to increase immune response in the subject. For example, factors produced by the target cell upon expression of the thanotransmission polypeptides may induce an immuno-stimulatory response (e.g., a pro-inflammatory response) in a responding cell (e.g., an immune cell). In one embodiment, the immune response is an anti-cancer response.

According to the methods of the disclosure, immune activity may be modulated by interaction of the target cell with a broad range of immune cells, including, for example, any one or more of mast cells, Natural Killer (NK) cells, basophils, neutrophils, monocytes, macrophages, dendritic cells, eosinophils, lymphocytes (e.g. B-lymphocytes (B-cells)), and T-lymphocytes (T-cells)).

### Types of Immune Cells

Mast cells are a type of granulocyte containing granules rich in histamine and heparin, an anti-coagulant. When activated, a mast cell releases inflammatory compounds from the granules into the local microenvironment. Mast cells play a role in allergy, anaphylaxis, wound healing, angiogenesis, immune tolerance, defense against pathogens, and blood-brain barrier function.

Natural Killer (NK) cells are cytotoxic lymphocytes that lyse certain tumor and virus infected cells without any prior stimulation or immunization. NK cells are also potent producers of various cytokines, e.g. IFN-gamma (IFNγ), TNF-alpha (TNFα), GM-CSF and IL-3. Therefore, NK cells are also believed to function as regulatory cells in the immune system, influencing other cells and responses. In humans, NK cells are broadly defined as CD56+CD3− lymphocytes. The cytotoxic activity of NK cells is tightly controlled by a balance between the activating and inhibitory signals from receptors on the cell surface. A main group of receptors that inhibits NK cell activation are the inhibitory killer immunoglobulin-like receptors (KIRs). Upon recognition of self MHC class I molecules on the target cells, these receptors deliver an inhibitory signal that stops the activating signaling cascade, keeping cells with normal MHC class I expression from NK cell lysis. Activating receptors include the natural cytotoxicity receptors (NCR) and NKG2D that push the balance towards cytolytic action through engagement with different ligands on the target cell surface. Thus, NK cell recognition of target cells is tightly regulated by processes involving the integration of signals delivered from multiple activating and inhibitory receptors.

Monocytes are bone marrow-derived mononuclear phagocyte cells that circulate in the blood for few hours/days before being recruited into tissues. See Wacleche et al., 2018, Viruses (10)2: 65. The expression of various chemokine receptors and cell adhesion molecules at their surface allows them to exit the bone marrow into the blood and to be subsequently recruited from the blood into tissues. Monocytes belong to the innate arm of the immune system providing responses against viral, bacterial, fungal or parasitic infections. Their functions include the killing of pathogens via phagocytosis, the production of reactive oxygen species (ROS), nitric oxide (NO), myeloperoxidase and inflammatory cytokines. Under specific conditions, monocytes can stimulate or inhibit T-cell responses during cancer as well as infectious and autoimmune diseases. They are also involved in tissue repair and neovascularization.

Macrophages engulf and digest substances such as cellular debris, foreign substances, microbes and cancer cells in a process called phagocytosis. Besides phagocytosis, macrophages play a critical role in nonspecific defense (innate immunity) and also help initiate specific defense mechanisms (adaptive immunity) by recruiting other immune cells such as lymphocytes. For example, macrophages are important as antigen presenters to T cells. Beyond increasing inflammation and stimulating the immune system, macrophages also play an important anti-inflammatory role and can decrease immune reactions through the release of cytokines. Macrophages that encourage inflammation are called M1 macrophages, whereas those that decrease inflammation and encourage tissue repair are called M2 macrophages.

Dendritic cells (DCs) play a critical role in stimulating immune responses against pathogens and maintaining immune homeostasis to harmless antigens. DCs represent a heterogeneous group of specialized antigen-sensing and antigen-presenting cells (APCs) that are essential for the induction and regulation of immune responses. In the peripheral blood, human DCs are characterized as cells lacking the T-cell (CD3, CD4, CD8), the B-cell (CD19, CD20) and the monocyte markers (CD14, CD16) but highly expressing HLA-DR and other DC lineage markers (e.g., CD1a, CD1c). See Murphy et al., Janeway's Immunobiology. 8th ed. Garland Science; New York, NY, USA: 2012. 868p.

The term “lymphocyte” refers to a small white blood cell formed in lymphatic tissue throughout the body and in normal adults making up about 22-28% of the total number of leukocytes in the circulating blood that plays a large role in defending the body against disease. Individual lymphocytes are specialized in that they are committed to respond to a limited set of structurally related antigens through recombination of their genetic material (e.g. to create a T cell receptor and a B cell receptor). This commitment, which exists before the first contact of the immune system with a given antigen, is expressed by the presence of receptors specific for determinants (epitopes) on the antigen on the lymphocyte's surface membrane. Each lymphocyte possesses a unique population of receptors, all of which have identical combining sites. One set, or clone, of lymphocytes differs from another clone in the structure of the combining region of its receptors and thus differs in the epitopes that it can recognize. Lymphocytes differ from each other not only in the specificity of their receptors, but also in their functions. (Paul, W. E., “Chapter 1: The immune system: an introduction,” Fundamental Immunology, 4th Edition, Ed. Paul, W. E., Lippicott-Raven Publishers, Philadelphia, (1999), at p. 102).

Lymphocytes include B-lymphocytes (B-cells), which are precursors of antibody-secreting cells, and T-lymphocytes (T-cells).

B-Lymphocytes (B-Cells)

B-lymphocytes are derived from hematopoietic cells of the bone marrow. A mature B-cell can be activated with an antigen that expresses epitopes that are recognized by its cell surface. The activation process may be direct, dependent on cross-linkage of membrane Ig molecules by the antigen (cross-linkage-dependent B-cell activation), or indirect, via interaction with a helper T-cell, in a process referred to as cognate help. In many physiological situations, receptor cross-linkage stimuli and cognate help synergize to yield more vigorous B-cell responses (Paul, W. E., “Chapter 1: The immune system: an introduction,” Fundamental Immunology, 4th Edition, Ed. Paul, W. E., Lippicott-Raven Publishers, Philadelphia, (1999)).

Cross-linkage dependent B-cell activation requires that the antigen express multiple copies of the epitope complementary to the binding site of the cell surface receptors, because each B-cell expresses Ig molecules with identical variable regions. Such a requirement is fulfilled by other antigens with repetitive epitopes, such as capsular polysaccharides of microorganisms or viral envelope proteins. Cross-linkage-dependent B-cell activation is a major protective immune response mounted against these microbes (Paul, W. E., “Chapter 1: The immune system: an introduction”, Fundamental Immunology, 4th Edition, Ed. Paul, W. E., Lippicott-Raven Publishers, Philadelphia, (1999)).

Cognate help allows B-cells to mount responses against antigens that cannot cross-link receptors and, at the same time, provides costimulatory signals that rescue B cells from inactivation when they are stimulated by weak cross-linkage events. Cognate help is dependent on the binding of antigen by the B-cell's membrane immunoglobulin (Ig), the endocytosis of the antigen, and its fragmentation into peptides within the endosomal/lysosomal compartment of the cell. Some of the resultant peptides are loaded into a groove in a specialized set of cell surface proteins known as class II major histocompatibility complex (MHC) molecules. The resultant class II/peptide complexes are expressed on the cell surface and act as ligands for the antigen-specific receptors of a set of T-cells designated as CD4⁺ T-cells. The CD4⁺ T-cells bear receptors on their surface specific for the B-cell's class II/peptide complex. B-cell activation depends not only on the binding of the T cell through its T cell receptor (TCR), but this interaction also allows an activation ligand on the T-cell (CD40 ligand) to bind to its receptor on the B-cell (CD40) signaling B-cell activation. In addition, T helper cells secrete several cytokines that regulate the growth and differentiation of the stimulated B-cell by binding to cytokine receptors on the B cell (Paul, W. E., “Chapter 1: The immune system: an introduction, “Fundamental Immunology, 4th Edition, Ed. Paul, W. E., Lippicott-Raven Publishers, Philadelphia, (1999)).

During cognate help for antibody production, the CD40 ligand is transiently expressed on activated CD4⁺ T helper cells, and it binds to CD40 on the antigen-specific B cells, thereby transducing a second costimulatory signal. The latter signal is essential for B cell growth and differentiation and for the generation of memory B cells by preventing apoptosis of germinal center B cells that have encountered antigen. Hyperexpression of the CD40 ligand in both B and T cells is implicated in pathogenic autoantibody production in human SLE patients (Desai-Mehta, A. et al., “Hyperexpression of CD40 ligand by B and T cells in human lupus and its role in pathogenic autoantibody production,” J. Clin. Invest. Vol. 97(9), 2063-2073, (1996)).

T-Lymphocytes (T-Cells)

T-lymphocytes derived from precursors in hematopoietic tissue, undergo differentiation in the thymus, and are then seeded to peripheral lymphoid tissue and to the recirculating pool of lymphocytes. T-lymphocytes or T cells mediate a wide range of immunologic functions. These include the capacity to help B cells develop into antibody-producing cells, the capacity to increase the microbicidal action of monocytes/macrophages, the inhibition of certain types of immune responses, direct killing of target cells, and mobilization of the inflammatory response. These effects depend on T cell expression of specific cell surface molecules and the secretion of cytokines (Paul, W. E., “Chapter 1: The immune system: an introduction”, Fundamental Immunology, 4th Edition, Ed. Paul, W. E., Lippicott-Raven Publishers, Philadelphia, (1999)).

T cells differ from B cells in their mechanism of antigen recognition. Immunoglobulin, the B cell's receptor, binds to individual epitopes on soluble molecules or on particulate surfaces. B-cell receptors see epitopes expressed on the surface of native molecules. While antibody and B-cell receptors evolved to bind to and to protect against microorganisms in extracellular fluids, T cells recognize antigens on the surface of other cells and mediate their functions by interacting with, and altering, the behavior of these antigen-presenting cells (APCs). There are three main types of APCs in peripheral lymphoid organs that can activate T cells: dendritic cells, macrophages and B cells. The most potent of these are the dendritic cells, whose only function is to present foreign antigens to T cells. Immature dendritic cells are located in tissues throughout the body, including the skin, gut, and respiratory tract. When they encounter invading microbes at these sites, they endocytose the pathogens and their products, and carry them via the lymph to local lymph nodes or gut associated lymphoid organs. The encounter with a pathogen induces the dendritic cell to mature from an antigen-capturing cell to an APC that can activate T cells. APCs display three types of protein molecules on their surface that have a role in activating a T cell to become an effector cell: (1) MHC proteins, which present foreign antigen to the T cell receptor; (2) costimulatory proteins which bind to complementary receptors on the T cell surface; and (3) cell-cell adhesion molecules, which enable a T cell to bind to the APC for long enough to become activated (“Chapter 24: The adaptive immune system,” Molecular Biology of the Cell, Alberts, B. et al., Garland Science, NY, (2002)).

T-cells are subdivided into two distinct classes based on the cell surface receptors they express. The majority of T cells express T cell receptors (TCR) consisting of α and β-chains. A small group of T cells express receptors made of γ and δ chains. Among the α/β T cells are two sub-lineages: those that express the coreceptor molecule CD4 (CD4⁺ T cells); and those that express CD8 (CD8⁺ T cells). These cells differ in how they recognize antigen and in their effector and regulatory functions.

CD4⁺ T cells are the major regulatory cells of the immune system. Their regulatory function depends both on the expression of their cell-surface molecules, such as CD40 ligand whose expression is induced when the T cells are activated, and the wide array of cytokines they secrete when activated.

T cells also mediate important effector functions, some of which are determined by the patterns of cytokines they secrete. The cytokines can be directly toxic to target cells and can mobilize potent inflammatory mechanisms.

In addition, T cells, particularly CD8⁺ T cells, can develop into cytotoxic T-lymphocytes (CTLs) capable of efficiently lysing target cells that express antigens recognized by the CTLs (Paul, W. E., “Chapter 1: The immune system: an introduction,” Fundamental Immunology, 4th Edition, Ed. Paul, W. E., Lippicott-Raven Publishers, Philadelphia, (1999)).

T cell receptors (TCRs) recognize a complex consisting of a peptide derived by proteolysis of the antigen bound to a specialized groove of a class II or class I MHC protein. CD4⁺ T cells recognize only peptide/class II complexes while CD8⁺ T cells recognize peptide/class I complexes (Paul, W. E., “Chapter 1: The immune system: an introduction,” Fundamental Immunology, 4th Edition, Ed. Paul, W. E., Lippicott-Raven Publishers, Philadelphia, (1999)).

The TCR's ligand (i.e., the peptide/MHC protein complex) is created within APCs. In general, class II MHC molecules bind peptides derived from proteins that have been taken up by the APC through an endocytic process. These peptide-loaded class II molecules are then expressed on the surface of the cell, where they are available to be bound by CD4⁺ T cells with TCRs capable of recognizing the expressed cell surface complex. Thus, CD4⁺ T cells are specialized to react with antigens derived from extracellular sources (Paul, W. E., “Chapter 1: The immune system: an introduction,” Fundamental Immunology, 4th Edition, Ed. Paul, W. E., Lippicott-Raven Publishers, Philadelphia, (1999)).

In contrast, class I MHC molecules are mainly loaded with peptides derived from internally synthesized proteins, such as viral proteins. These peptides are produced from cytosolic proteins by proteolysis by the proteosome and are translocated into the rough endoplasmic reticulum. Such peptides, generally composed of nine amino acids in length, are bound into the class I MHC molecules and are brought to the cell surface, where they can be recognized by CD8⁺ T cells expressing appropriate receptors. This gives the T cell system, particularly CD8⁺ T cells, the ability to detect cells expressing proteins that are different from, or produced in much larger amounts than, those of cells of the remainder of the organism (e.g., viral antigens) or mutant antigens (such as active oncogene products), even if these proteins in their intact form are neither expressed on the cell surface nor secreted (Paul, W. E., “Chapter 1: The immune system: an introduction,” Fundamental Immunology, 4th Edition, Ed. Paul, W. E., Lippicott-Raven Publishers, Philadelphia, (1999)).

T cells can also be classified based on their function as helper T cells; T cells involved in inducing cellular immunity; suppressor T cells; and cytotoxic T cells.

Helper T Cells

Helper T cells are T cells that stimulate B cells to make antibody responses to proteins and other T cell-dependent antigens. T cell-dependent antigens are immunogens in which individual epitopes appear only once or a limited number of times such that they are unable to cross-link the membrane immunoglobulin (Ig) of B cells or do so inefficiently. B cells bind the antigen through their membrane Ig, and the complex undergoes endocytosis. Within the endosomal and lysosomal compartments, the antigen is fragmented into peptides by proteolytic enzymes, and one or more of the generated peptides are loaded into class II MHC molecules, which traffic through this vesicular compartment. The resulting peptide/class II MHC complex is then exported to the B-cell surface membrane. T cells with receptors specific for the peptide/class II molecular complex recognize this complex on the B-cell surface. (Paul, W. E., “Chapter 1: The immune system: an introduction,” Fundamental Immunology, 4th Edition, Ed. Paul, W. E., Lippicott-Raven Publishers, Philadelphia (1999)).

B-cell activation depends both on the binding of the T cell through its TCR and on the interaction of the T-cell CD40 ligand (CD40L) with CD40 on the B cell. T cells do not constitutively express CD40L. Rather, CD40L expression is induced as a result of an interaction with an APC that expresses both a cognate antigen recognized by the TCR of the T cell and CD80 or CD86. CD80/CD86 is generally expressed by activated, but not resting, B cells so that the helper interaction involving an activated B cell and a T cell can lead to efficient antibody production. In many cases, however, the initial induction of CD40L on T cells is dependent on their recognition of antigen on the surface of APCs that constitutively express CD80/86, such as dendritic cells. Such activated helper T cells can then efficiently interact with and help B cells. Cross-linkage of membrane Ig on the B cell, even if inefficient, may synergize with the CD40L/CD40 interaction to yield vigorous B-cell activation. The subsequent events in the B-cell response, including proliferation, Ig secretion, and class switching of the Ig class being expressed, either depend or are enhanced by the actions of T cell-derived cytokines (Paul, W. E., “Chapter 1: The immune system: an introduction,” Fundamental Immunology, 4th Edition, Ed. Paul, W. E., Lippicott-Raven Publishers, Philadelphia, (1999)).

CD4⁺ T cells tend to differentiate into cells that principally secrete the cytokines IL-4, IL-5, IL-6, and IL-10 (T_{H}2 cells) or into cells that mainly produce IL-2, IFN-γ, and lymphotoxin (T_{H}1 cells). The T_{H}2 cells are very effective in helping B-cells develop into antibody-producing cells, whereas the T_{H}1 cells are effective inducers of cellular immune responses, involving enhancement of microbicidal activity of monocytes and macrophages, and consequent increased efficiency in lysing microorganisms in intracellular vesicular compartments. Although CD4⁺ T cells with the phenotype of T_{H}2 cells (i.e., IL-4, IL-5, IL-6 and IL-10) are efficient helper cells, T_{H}1 cells also have the capacity to be helpers (Paul, W. E., “Chapter 1: The immune system: an introduction, “Fundamental Immunology, 4th Edition, Ed. Paul, W. E., Lippicott-Raven Publishers, Philadelphia, (1999)).

T Cell Involvement in Cellular Immunity Induction

T cells also may act to enhance the capacity of monocytes and macrophages to destroy intracellular microorganisms. In particular, interferon-gamma (IFN-γ) produced by helper T cells enhances several mechanisms through which mononuclear phagocytes destroy intracellular bacteria and parasitism including the generation of nitric oxide and induction of tumor necrosis factor (TNF) production. T_{H1} cells are effective in enhancing the microbicidal action, because they produce IFN-γ. In contrast, two of the major cytokines produced by T_{H2} cells, IL-4 and IL-10, block these activities (Paul, W. E., “Chapter 1: The immune system: an introduction,” Fundamental Immunology, 4th Edition, Ed. Paul, W. E., Lippicott-Raven Publishers, Philadelphia, (1999)).

Regulatory T (Treg) Cells

Immune homeostasis is maintained by a controlled balance between initiation and downregulation of the immune response. The mechanisms of both apoptosis and T cell anergy (a tolerance mechanism in which the T cells are intrinsically functionally inactivated following an antigen encounter (Schwartz, R. H., “T cell anergy”, Annu. Rev. Immunol., Vol. 21: 305-334 (2003)) contribute to the downregulation of the immune response. A third mechanism is provided by active suppression of activated T cells by suppressor or regulatory CD4+T (Treg) cells (Reviewed in Kronenberg, M. et al., “Regulation of immunity by self-reactive T cells”, Nature, Vol. 435: 598-604 (2005)). CD4⁺ Tregs that constitutively express the IL-2 receptor alpha (IL-2Ru) chain (CD4+CD25+) are a naturally occurring T cell subset that are anergic and suppressive (Taams, L. S. et al., “Human anergic/suppressive CD4⁺CD25⁺ T cells: a highly differentiated and apoptosis-prone population”, Eur. J. Immunol. Vol. 31: 1122-1131 (2001)). Human CD4+CD25+ Tregs, similar to their murine counterpart, are generated in the thymus and are characterized by the ability to suppress proliferation of responder T cells through a cell-cell contact-dependent mechanism, the inability to produce IL-2, and the anergic phenotype in vitro. Human CD4⁺CD25⁺ T cells can be split into suppressive (CD25^{high}) and nonsuppressive (CD25^{low}) cells, according to the level of CD25 expression. A member of the forkhead family of transcription factors, FOXP3, has been shown to be expressed in murine and human CD4⁺CD25⁺ Tregs and appears to be a master gene controlling CD4⁺CD25⁺ Treg development (Battaglia, M. et al., “Rapamycin promotes expansion of functional CD4⁺CD25⁺Foxp3⁺ regulator T cells of both healthy subjects and type 1 diabetic patients”, J. Immunol., Vol. 177: 8338-8347, (2006)). Accordingly, in some embodiments, an increase in immune response may be associated with a lack of activation or proliferation of regulatory T cells.

Cytotoxic T Lymphocytes

CD8⁺ T cells that recognize peptides from proteins produced within the target cell have cytotoxic properties in that they lead to lysis of the target cells. The mechanism of CTL-induced lysis involves the production by the CTL of perform, a molecule that can insert into the membrane of target cells and promote the lysis of that cell. Perforin-mediated lysis is enhanced by granzymes, a series of enzymes produced by activated CTLs. Many active CTLs also express large amounts of fas ligand on their surface. The interaction of fas ligand on the surface of CTL with fas on the surface of the target cell initiates apoptosis in the target cell, leading to the death of these cells. CTL-mediated lysis appears to be a major mechanism for the destruction of virally infected cells.

Lymphocyte Activation

The term “activation” or “lymphocyte activation” refers to stimulation of lymphocytes by specific antigens, nonspecific mitogens, or allogeneic cells resulting in synthesis of RNA, protein and DNA and production of lymphokines; it is followed by proliferation and differentiation of various effector and memory cells. T-cell activation is dependent on the interaction of the TCR/CD3 complex with its cognate ligand, a peptide bound in the groove of a class I or class II MHC molecule. The molecular events set in motion by receptor engagement are complex. Among the earliest steps appears to be the activation of tyrosine kinases leading to the tyrosine phosphorylation of a set of substrates that control several signaling pathways. These include a set of adapter proteins that link the TCR to the ras pathway, phospholipase Cγ1, the tyrosine phosphorylation of which increases its catalytic activity and engages the inositol phospholipid metabolic pathway, leading to elevation of intracellular free calcium concentration and activation of protein kinase C, and a series of other enzymes that control cellular growth and differentiation. Full responsiveness of a T cell requires, in addition to receptor engagement, an accessory cell-delivered costimulatory activity, e.g., engagement of CD28 on the T cell by CD80 and/or CD86 on the APC.

T-Memory Cells

Following the recognition and eradication of pathogens through adaptive immune responses, the vast majority (90-95%) of T cells undergo apoptosis with the remaining cells forming a pool of memory T cells, designated central memory T cells (TCM), effector memory T cells (TEM), and resident memory T cells (TRM) (Clark, R. A., “Resident memory T cells in human health and disease”, Sci. Transl. Med., 7, 269rv1, (2015)).

Compared to standard T cells, these memory T cells are long-lived with distinct phenotypes such as expression of specific surface markers, rapid production of different cytokine profiles, capability of direct effector cell function, and unique homing distribution patterns. Memory T cells exhibit quick reactions upon re-exposure to their respective antigens in order to eliminate the reinfection of the offender and thereby restore balance of the immune system rapidly. Increasing evidence substantiates that autoimmune memory T cells hinder most attempts to treat or cure autoimmune diseases (Clark, R. A., “Resident memory T cells in human health and disease”, Sci. Transl. Med., Vol. 7, 269rv1, (2015)).

### Increasing Immune Activity

The IL-12 and thanotransmission polypeptides as described herein may increase immune activity in a tissue or subject by increasing the level or activity of any one or more of the immune cells described herein, for example, macrophages, monocytes, dendritic cells, B-cells, T-cells, and CD4+, CD8+ or CD3+ cells (e.g. CD4+, CD8+ or CD3+ T cells) in the tissue or subject. For example, in one embodiment, one or more recombinant nucleic acid molecules encoding thanotransmission polypeptides as described herein is administered in an amount sufficient to increase in a tissue or subject one or more of the level or activity of macrophages, the level or activity of monocytes, the level or activity of dendritic cells, the level or activity of T-cells, the level or activity of NK cells, the level or activity of B-cells, and the level or activity of CD4+, CD8+ or CD3+ cells (e.g. CD4+, CD8+ or CD3+ T cells).

In some aspects, the disclosure relates to a method of increasing the level or activity of macrophages, monocytes, NK cells, B-cells, T-cells and/or dendritic cells in a tissue or subject, comprising administering to the tissue or subject, a recombinant nucleic acid molecule, vector (e.g. an engineered virus, plasmid or transposon), cell, or pharmaceutical composition as described herein, wherein the recombinant nucleic acid molecule, vector, cell, or pharmaceutical composition is administered in an amount sufficient to increase the level or activity of macrophages, monocytes, NK cells, B-cells, T cells and/or dendritic cells relative to a tissue or subject that is not treated with the nucleic acid molecule, vector, cell, or pharmaceutical composition.

In one embodiment, the subject is in need of an increased level or activity of macrophages, monocytes, dendritic cells, NK cells, B-cells, and/or T-cells.

In one embodiment, the level or activity of macrophages, monocytes, NK cells, B-cells, T-cells or dendritic cells is increased by at least 10%, 20%, 30%, 40%, 50%, 60%, 70%, 80%, 90% or 100%, or by at least 2-fold, 4-fold, 6-fold, 8-fold, or 10-fold relative to a tissue or subject that is not treated with the engineered virus.

In some aspects, the disclosure relates to a method of increasing the level or activity of CD4+, CD8+, or CD3+ cells in a tissue or subject, comprising administering to the subject a recombinant nucleic acid molecule, vector (e.g. an engineered virus, plasmid or transposon), cell, or pharmaceutical composition as described herein in an amount sufficient to increase the level or activity of CD4+, CD8+, or CD3+ cells relative to a tissue or subject that is not treated with the recombinant nucleic acid molecule, vector, cell, or pharmaceutical composition.

In one embodiment, the subject is in need of an increased level or activity of CD4+, CD8+, or CD3+ cells.

In one embodiment, the level or activity of CD4+, CD8+, or CD3+ cells is increased by at least 10%, 20%, 30%, 40%, 50%, 60%, 70%, 80%, 90% or 100%, or by at least 2-fold, 4-fold, 6-fold, 8-fold, or 10-fold relative to a tissue or subject that is not treated with the engineered virus.

The IL-12 and thanotransmission polypeptides may also increase immune activity in a cell, tissue or subject by increasing the level or activity of a pro-immune cytokine produced by an immune cell. For example, in some embodiments, the recombinant nucleic acid molecule, vector (e.g. an engineered virus, plasmid or transposon), cell, or pharmaceutical composition as described herein is administered in an amount sufficient to increase in a cell, tissue or subject the level or activity of a pro-immune cytokine produced by an immune cell. In one embodiment, the pro-immune cytokine is selected from IFN-α, IL-1, IL-12, IL-18, IL-2, IL-15, IL-4, IL-6, TNF-α, IL-17, KC, MCP-1, RANTES, IL-1β, IP-10, IL-10, IFN-β, and GMCSF.

In some aspects, the disclosure relates to a method of inducing pro-inflammatory transcriptional responses in the immune cells described herein, e.g. inducing NFkB pathways, interferon IRF signaling, and/or STAT signaling in an immune cell in a tissue or subject, comprising administering to the tissue or subject, a nucleic acid molecule, vector (e.g. an engineered virus, plasmid or transposon), cell, or pharmaceutical composition as described herein in an amount sufficient to induce pro-inflammatory transcriptional responses in the immune cells NFkB pathways, interferon IRF signaling, and/or STAT signaling in an immune cell.

The IL-12 and thanotransmission polypeptides may also increase immune activity in a cell, tissue or subject by modulation of signaling through intracellular sensors of nucleic acids, e.g. stimulator of interferon genes (STING).

In some aspects, the disclosure relates to a method of increasing immune activity in a cell, tissue or subject by modulation of signaling through intracellular sensors of nucleic acids, e.g. stimulator of interferon genes (STING), comprising administering to the tissue or subject, a recombinant nucleic acid molecule, vector (e.g. an engineered virus, plasmid or transposon), cell, or pharmaceutical composition as described herein in an amount sufficient to increase immune activity in a cell, tissue or subject by modulation of signaling through intracellular sensors of nucleic acids, e.g. stimulator of interferon genes (STING).

The thanotransmission polypeptides may also increase immune activity in a cell, tissue or subject by inducing pro-inflammatory transcriptional responses in the immune cells described herein, e.g. inducing nuclear factor kappa-light-chain-enhancer of activated B cells (NFkB) pathways, interferon regulatory factor (IRF) signaling, and/or STAT signaling. For example, in some embodiments, the recombinant nucleic acid molecule, vector (e.g. an engineered virus, plasmid or transposon), cell, or pharmaceutical composition as described herein is administered in an amount sufficient to induce NFkB pathways, interferon IRF signaling, and/or STAT signaling in an immune cell.

In some aspects, the disclosure relates to a method of inducing pro-inflammatory transcriptional responses in the immune cells described herein, e.g. inducing NFkB pathways, interferon IRF signaling, and/or STAT signaling in an immune cell in a tissue or subject, comprising administering to the tissue or subject, a recombinant nucleic acid molecule, vector (e.g. an engineered virus, plasmid or transposon), cell, or pharmaceutical composition as described herein, wherein the recombinant nucleic acid molecule, vector, cell, or pharmaceutical composition is administered in an amount sufficient to induce pro-inflammatory transcriptional responses in the immune cells NFkB pathways, interferon IRF signaling, and/or STAT signaling in an immune cell. In a particular embodiment, increasing immune response comprises increasing IRF activity. In a particular embodiment, increasing immune response comprises increasing NFkB activity.

The IL-12 and thanotransmission polypeptides may also increase immune activity in a tissue or subject by induction or modulation of an antibody response. For example, in some embodiments, the recombinant nucleic acid molecule, vector (e.g. an engineered virus, plasmid or transposon), cell, or pharmaceutical composition as described herein is administered in an amount sufficient to induce or modulate an antibody response in the tissue or subject.

In some aspects, the disclosure relates to a method of increasing immune activity in a tissue or subject by induction or modulation of an antibody response in an immune cell in a tissue or subject, comprising administering to the tissue or subject, a recombinant nucleic acid molecule, vector (e.g. an engineered virus, plasmid or transposon), cell, or pharmaceutical composition as described herein, wherein the recombinant nucleic acid molecule, vector, cell, or pharmaceutical composition is administered in an amount sufficient to increase immune activity in the tissue or subject relative to a tissue or subject that is not treated with the nucleic acid molecule, vector, cell, or pharmaceutical composition.

In some aspects, the disclosure relates to a method of increasing the level or activity of a pro-immune cytokine in a cell, tissue or subject, comprising administering to the cell, tissue or subject a recombinant nucleic acid molecule, vector (e.g., an engineered virus, plasmid or transposon), cell, or pharmaceutical composition as described herein, wherein the recombinant nucleic acid molecule, vector, cell, or pharmaceutical composition is administered in an amount sufficient to increase the level or activity of the pro-immune cytokine relative to a cell, tissue or subject that is not treated with the nucleic acid molecule, vector, cell, or pharmaceutical composition.

In one embodiment, the subject is in need of an increased level or activity of a pro-immune cytokine.

In one embodiment, the level or activity of the pro-immune cytokine is increased by at least 10%, 20%, 30%, 40%, 50%, 60%, 70%, 80%, 90% or 100%, or by at least 2-fold, 4-fold, 6-fold, 8-fold, or 10-fold relative to a cell, tissue or subject that is not treated with the nucleic acid molecule, vector, cell, or pharmaceutical composition.

In one embodiment, the pro-immune cytokine is selected from IFN-α, IL-1, IL-12, IL-18, IL-2, IL-15, IL-4, IL-6, TNF-α, IL-17, KC, MCP-1, RANTES, IL-1β, IP-10, IL-10, IFN-β, and GMCSF.

In some embodiments, the increasing immune response comprises increasing chemokine production and/or activity. In some embodiments, the increasing immune response comprises increasing immune cell mediated cytotoxicity. In some embodiments, the increasing immune response comprises increasing expression of a receptor-ligand pairing. In some embodiments, the receptor-ligand pairing is selected from the group consisting of co-stimulatory molecules CD80 and CD86, FAS:FASL, and CD40L:CD40. In some embodiments, the increasing immune response comprises reducing anti-inflammatory signals and/or anti-inflammatory cells. In some embodiments, the anti-inflammatory signals are selected from IL-10 and TGF beta. In some embodiments, the anti-inflammatory cells are selected from T regulatory cells and myeloid derived suppressor cells. In some embodiments, the increased immune response comprises increased MHC I expression or increased HLA/MHC antigen presentation or antigen release by target cells. In some embodiments, the target cells are tumor cells. In some embodiments, the increased immune response comprises reduced expression of anti-immune factors. In some embodiments, the anti-immune factors are selected from the group consisting of immune checkpoint molecules, suppressive cytokines, and suppressive transcription factors. In some embodiments, the immune checkpoint molecule is PDL1. In some embodiments, the suppressive transcription factor is selected from SOCS1 and SOCS3.

The combination of two or more thanotransmission polypeptides as described herein may increase immune response relative to each thanotransmission polypeptide alone. In addition, the combination of IL-12 and one or more thanotransmission polypeptides as described herein may increase immune response relative to IL-12 alone or the one or more thanotransmission polypeptides alone. For example, in some embodiments, administration of the recombinant nucleic acid molecule encoding two or more different thanotransmission polypeptides to a subject increases immune response (e.g, increases NFkB activity, increases IRF activity, and/or increases the level or activity of any one or more of the immune cells described herein, for example, macrophages, monocytes, dendritic cells, NK cells, B-cells, T-cells, and CD4+, CD8+ or CD3+ cells (e.g. CD4+, CD8+ or CD3+ T cells) relative to a subject that is administered a nucleic acid molecule encoding only one of the thanotransmission polypeptides (e.g., only TRIF, or only a polypeptide selected from RIPK3, Gasdermin E, vICA, Npro, A238L, vMLKL, and variants thereof, or a dominant negative variant of IKBa). For example, in some embodiments, administering a recombinant nucleic acid molecule encoding TRIF and RIPK3 increases immune response (e.g, increases NFkB activity, increases IRF activity, and/or increases the level or activity of any one or more of the immune cells described herein, for example, macrophages, monocytes, dendritic cells, NK cells, B-cells, T-cells, and CD4+, CD8+ or CD3+ cells (e.g. CD4+, CD8+ or CD3+ T cells) relative to a subject that is administered a recombinant nucleic acid molecule encoding TRIF alone and/or relative to subject that is administered a recombinant nucleic acid molecule encoding RIPK3 alone.

For example, in some embodiments, administering a recombinant nucleic acid molecule encoding TRIF and RIPK3 increases immune response (e.g, increases NFkB activity, increases IRF activity, and/or increases the level or activity of any one or more of the immune cells described herein, for example, macrophages, monocytes, dendritic cells, NK cells, B-cells, T-cells, and CD4+, CD8+ or CD3+ cells (e.g. CD4+, CD8+ or CD3+ T cells) relative to a subject that is administered a recombinant nucleic acid molecule encoding TRIF alone and/or relative to subject that is administered a recombinant nucleic acid molecule encoding RIPK3 alone.

Addition of a third thanotransmission polypeptide may increase immune response relative to the two or more thanotransmission polypeptides alone. For example, in some embodiments, administration to a subject of a recombinant nucleic acid molecule encoding two or more different thanotransmission polypeptides as described herein and further encoding a third thanotransmission polypeptide selected from RIPK3, Gasdermin E, vICA, Npro, A238L, vMLKL, and variants thereof or a dominant negative variant of IKBa increases immune response (e.g., increases NFkB activity, increases IRF activity, and/or increases the level or activity of any one or more of the immune cells described herein, for example, macrophages, monocytes, dendritic cells, NK cells, B-cells, T-cells, and CD4+, CD8+ or CD3+ cells (e.g. CD4+, CD8+ or CD3+ T cells) relative to a subject that is administered a nucleic acid molecule that encodes the two or more different thanotransmission polypeptides, but does not further encode the third thanotransmission polypeptide. For example, in some embodiments, administration of a recombinant nucleic acid molecule encoding TRIF, RIPK3 and vICA increases immune response (e.g., increases NFkB activity, increases IRF activity, and/or increases the level or activity of any one or more of the immune cells described herein, for example, macrophages, monocytes, dendritic cells, NK cells, B-cells, T-cells, and CD4+, CD8+ or CD3+ cells (e.g. CD4+, CD8+ or CD3+ T cells)) relative to a subject that is administered a nucleic acid molecule that encodes TRIF and RIPK3, but does not encode vICA. In some embodiments, administration of a recombinant nucleic acid molecule encoding TRIF, RIPK3 and vICA increases immune response (e.g., increases IRF activity) relative to a subject that is administered a nucleic acid molecule that encodes TRIF and RIPK3, but does not encode vICA.

Inclusion of Gasdermin E in the two or more thanotransmission polypeptides may increase immune response relative to one or more thanotransmission polypeptides that do not include Gasdermin E. For example, in some embodiments, administration of a recombinant nucleic acid molecule encoding Gasdermin E and one or more additional thanotransmission polypeptides increases immune response (e.g., increases NFkB activity, increases IRF activity, and/or increases the level or activity of any one or more of the immune cells described herein, for example, macrophages, monocytes, dendritic cells, NK cells, B-cells, T-cells, and CD4+, CD8+ or CD3+ cells (e.g. CD4+, CD8+ or CD3+ T cells)) relative to a nucleic acid molecule encoding the one or more additional thanotransmission polypeptides alone. In some embodiments, administration of a recombinant nucleic acid molecule encoding TRIF, RIPK3 and Gasdermin E increases immune response (e.g, increases NFkB activity, increases IRF activity, and/or increases the level or activity of any one or more of the immune cells described herein, for example, macrophages, monocytes, dendritic cells, NK cells, B-cells, T-cells, and CD4+, CD8+ or CD3+ cells (e.g. CD4+, CD8+ or CD3+ T cells)) relative to a nucleic acid molecule encoding TRIF and RIPK3 alone. In some embodiments, administration of a recombinant nucleic acid molecule encoding TRIF, RIPK3 and Gasdermin E increases IRF activity relative to a nucleic acid molecule encoding TRIF and RIPK3 alone.

Use of IL-12 in combination with one or more thanotransmission polypeptides may increase immune response relative to IL-12 alone. For example, in some embodiments, administration of one or more recombinant nucleic acid molecules encoding IL-12 and one or more additional thanotransmission polypeptides increases immune response (e.g., increases NFkB activity, increases IRF activity, and/or increases the level or activity of any one or more of the immune cells described herein, for example, macrophages (e.g., immune-stimulating macrophages), monocytes, dendritic cells, B-cells, NK cells, T-cells, and CD4+, CD8+ or CD3+ cells (e.g. CD4+, CD8+ or CD3+ T cells)) relative to a nucleic acid molecule encoding IL-12 alone, or relative to a nucleic acid molecule encoding the one or more additional thanotransmission polypeptides alone. In some embodiments, administration of one or more recombinant nucleic acid molecule encoding IL-12, TRIF and Gasdermin E increases immune response (e.g, increases NFkB activity, increases IRF activity, and/or increases the level or activity of any one or more of the immune cells described herein, for example, macrophages (e.g., immune-stimulating macrophages), monocytes, dendritic cells, B-cells, NK cells, T-cells, and CD4+, CD8+ or CD3+ cells (e.g. CD4+, CD8+ or CD3+ T cells)) relative to a nucleic acid molecule encoding IL-12 alone, or relative to one or more nucleic acid molecules encoding TRIF and Gasdermin E alone.

In some embodiments, the methods disclosed herein further include, before administration of the nucleic acid molecule, vector, cell, or pharmaceutical composition, evaluating the cell, tissue or subject for one or more of: the level or activity of macrophages; the level or activity of monocytes; the level or activity of dendritic cells; the level or activity of NK cells; the level or activity of CD4+ cells, CD8+ cells, or CD3+ cells; the level or activity of T cells; the level or activity of B cells, and the level or activity of a pro-immune cytokine.

In one embodiment, the methods of the invention further include, after administration of the recombinant nucleic acid molecule, vector (e.g. an engineered virus, plasmid or transposon), cell, or pharmaceutical composition as described herein, evaluating the cell, tissue or subject for one or more of: the level or activity of NFkB, IRF or STING; the level or activity of macrophages; the level or activity of monocytes; the level or activity of dendritic cells; the level or activity of NK cells; the level or activity of CD4+ cells, CD8+ cells or CD3+ cells; the level or activity of T cells; and the level or activity of a pro-immune cytokine.

Methods of measuring the level or activity of NFkB, IRF or STING; the level or activity of macrophages; the level or activity of monocytes; the level or activity of dendritic cells; the level or activity of NK cells; the level or activity of CD4+ cells, CD8+ cells or CD3+ cells; the level or activity of T cells; and the level or activity of a pro-immune cytokine are known in the art.

For example, the protein level or activity of NFkB, IRF or STING may be measured by suitable techniques known in the art including ELISA, Western blot or in situ hybridization. The level of a nucleic acid (e.g. an mRNA) encoding NFkB, IRF or STING may be measured using suitable techniques known in the art including polymerase chain reaction (PCR) amplification reaction, reverse-transcriptase PCR analysis, quantitative real-time PCR, single-strand conformation polymorphism analysis (SSCP), mismatch cleavage detection, heteroduplex analysis, Northern blot analysis, in situ hybridization, array analysis, deoxyribonucleic acid sequencing, restriction fragment length polymorphism analysis, and combinations or sub-combinations thereof.

Methods for measuring the level and activity of macrophages are described, for example, in Chitu et al., 2011, Curr Protoc Immunol 14: 1-33. The level and activity of monocytes may be measured by flow cytometry, as described, for example, in Henning et al., 2015, Journal of Immunological Methods 423: 78-84. The level and activity of dendritic cells may be measured by flow cytometry, as described, for example in Dixon et al., 2001, Infect Immun. 69(7): 4351-4357. Each of these references is incorporated by reference herein in its entirety.

The level or activity of T cells may be assessed using a human CD4+ T-cell-based proliferative assay. For example, cells are labeled with the fluorescent dye 5,6-carboxyfluorescein diacetate succinimidyl ester (CFSE). Those cells that proliferate show a reduction in CFSE fluorescence intensity, which is measured directly by flow cytometry. Alternatively, radioactive thymidine incorporation can be used to assess the rate of growth of the T cells.

In some embodiments, an increase in immune response may be associated with reduced activation of regulatory T cells (Tregs). Functional activity T regs may be assessed using an in vitro Treg suppression assay. Such an assay is described in Collinson and Vignali (Methods Mol Biol. 2011; 707: 21-37, incorporated by reference in its entirety herein).

The level or activity of a pro-immune cytokine may be quantified, for example, in CD8+ T cells. In embodiments, the pro-immune cytokine is selected from interferon alpha (IFN-α), interleukin-1 (IL-1), IL-12, IL-18, IL-2, IL-15, IL-4, IL-6, tumor necrosis factor alpha (TNF-α), IL-17, and granulocyte-macrophage colony-stimulating factor (GMCSF). Quantitation can be carried out using the ELISPOT (enzyme-linked immunospot) technique, that detects T cells that secrete a given cytokine (e.g. IFN-α) in response to an antigenic stimulation. T cells are cultured with antigen-presenting cells in wells which have been coated with, e.g., anti-IFN-α antibodies. The secreted IFN-α is captured by the coated antibody and then revealed with a second antibody coupled to a chromogenic substrate. Thus, locally secreted cytokine molecules form spots, with each spot corresponding to one IFN-α-secreting cell. The number of spots allows one to determine the frequency of IFN-α-secreting cells specific for a given antigen in the analyzed sample. The ELISPOT assay has also been described for the detection of TNF-α, interleukin-4 (IL-4), IL-6, IL-12, and GMCSF.

### VII. Methods of Treating Cancer

As provided herein, contacting a target cell with IL-12 and/or thanotransmission polypeptides, for example, through expression of the IL-12 and/or thanotransmission polypeptides in the target cell, can activate immune cells (e.g., T cells, B cells, NK cells, etc.) and, therefore, can enhance immune cell functions such as, for example, those involved in immunotherapies for treatment of cancer. Accordingly, in certain aspects, the disclosure relates to a method of treating a cancer in a subject in need thereof, the method comprising administering to the subject a recombinant nucleic acid molecule, vector (e.g. an engineered virus, plasmid or transposon), cell, or pharmaceutical composition as described herein to the subject in an amount and for a time sufficient to treat the cancer.

The ability of cancer cells to harness a range of complex, overlapping mechanisms to prevent the immune system from distinguishing self from non-self represents the fundamental mechanism of cancers to evade immunesurveillance. Mechanism(s) include disruption of antigen presentation, disruption of regulatory pathways controlling T cell activation or inhibition (immune checkpoint regulation), recruitment of cells that contribute to immune suppression (Tregs, MDSC) or release of factors that influence immune activity (IDO, PGE2). (See Harris et al., 2013, J Immunotherapy Cancer 1:12; Chen et al., 2013, Immunity 39:1; Pardoll, et al., 2012, Nature Reviews: Cancer 12:252; and Sharma et al., 2015, Cell 161:205, each of which is incorporated by reference herein in its entirety.)

Cancers for treatment using the methods described herein include, for example, all types of cancer or neoplasm or malignant tumors found in mammals, including, but not limited to: sarcomas, melanomas, carcinomas, leukemias, and lymphomas.

The term “sarcoma” generally refers to a tumor which is made up of a substance like the embryonic connective tissue and is generally composed of closely packed cells embedded in a fibrillar or homogeneous substance. Examples of sarcomas which can be treated with the methods of the invention include, for example, a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, uterine sarcoma, myxoid liposarcoma, leiomyosarcoma, spindle cell sarcoma, desmoplastic sarcoma, and telangiectaltic sarcoma.

The term “melanoma” is taken to mean a tumor arising from the melanocytic system of the skin and other organs. Melanomas which can be treated with the methods of the invention include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, and superficial spreading melanoma.

The term “carcinoma” refers to a malignant new growth made up of epithelial cells tending to infiltrate the surrounding tissues and give rise to metastases. Carcinomas which can be treated with the methods of the invention, as described herein, include, for example, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, colon adenocarcinoma of colon, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniform carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, merkel cell carcinoma, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, cervical squamous cell carcinoma, tonsil squamous cell carcinoma, and carcinoma villosum. In a particular embodiment, the cancer is renal cell carcinoma.

The term “leukemia” refers to a type of cancer of the blood or bone marrow characterized by an abnormal increase of immature white blood cells called “blasts”. Leukemia is a broad term covering a spectrum of diseases. In turn, it is part of the even broader group of diseases affecting the blood, bone marrow, and lymphoid system, which are all known as hematological neoplasms. Leukemias can be divided into four major classifications, acute lymphocytic (or lymphoblastic) leukemia (ALL), acute myelogenous (or myeloid or non-lymphatic) leukemia (AML), chronic lymphocytic leukemia (CLL), and chronic myelogenous leukemia (CML). Further types of leukemia include Hairy cell leukemia (HCL), T-cell prolymphocytic leukemia (T-PLL), large granular lymphocytic leukemia, and adult T-cell leukemia. In certain embodiments, leukemias include acute leukemias. In certain embodiments, leukemias include chronic leukemias.

The term “lymphoma” refers to a group of blood cell tumors that develop from lymphatic cells. The two main categories of lymphomas are Hodgkin lymphomas (HL) and non-Hodgkin lymphomas (NHL) Lymphomas include any neoplasms of the lymphatic tissues. The main classes are cancers of the lymphocytes, a type of white blood cell that belongs to both the lymph and the blood and pervades both.

In some embodiments, the recombinant nucleic acid molecules, vectors (e.g. an engineered virus, plasmid or transposon), cells and pharmaceutical compositions as describe herein are used for treatment of various types of solid tumors, for example breast cancer (e.g. triple negative breast cancer), bladder cancer, genitourinary tract cancer, colon cancer, rectal cancer, endometrial cancer, kidney (renal cell) cancer, pancreatic cancer, prostate cancer, thyroid cancer (e.g. papillary thyroid cancer), skin cancer, bone cancer, brain cancer, cervical cancer, liver cancer, stomach cancer, mouth and oral cancers, esophageal cancer, adenoid cystic cancer, neuroblastoma, testicular cancer, uterine cancer, thyroid cancer, head and neck cancer, kidney cancer, lung cancer (e.g. small cell lung cancer, non-small cell lung cancer), mesothelioma, ovarian cancer, sarcoma, stomach cancer, uterine cancer, cervical cancer, medulloblastoma, and vulvar cancer. In certain embodiments, skin cancer includes melanoma, squamous cell carcinoma, and cutaneous T-cell lymphoma (CTCL).

In a particular embodiment, the cancer may be a cancer that is “immunologically cold”, e.g., a tumor containing few infiltrating T cells, or a cancer that is not recognized and does not provoke a strong response by the immune system, making it difficult to treat with current immunotherapies. For example, in one embodiment, the cancer is selected from the group consisting of melanoma, cervical cancer, breast cancer, ovarian cancer, prostate cancer, testicular cancer, urothelial carcinoma, bladder cancer, non-small cell lung cancer, small cell lung cancer, sarcoma, colorectal adenocarcinoma, gastrointestinal stromal tumors, gastroesophageal carcinoma, colorectal cancer, pancreatic cancer, kidney cancer, malignant mesothelioma, leukemia, lymphoma, myelodysplasia syndrome, multiple myeloma, transitional cell carcinoma, neuroblastoma, plasma cell neoplasms, Wilm's tumor, and hepatocellular cancer (e.g. hepatocellular carcinoma).

In some embodiments, the cancer is a cancer that is responsive to an immunotherapy. For example, in some embodiments, the cancer is responsive to an immune checkpoint modulator therapy, e.g., an immune checkpoint inhibitor therapy. In some embodiments, the cancer that is responsive to an immunotherapy is selected from the group consisting of squamous cell head and neck cancer, melanoma, Merkel cell carcinoma, hepatocellular carcinoma, advanced renal cell carcinoma, metastatic microsatellite instability-high (MSI-H) or mismatch repair deficient (dMMR) cancers (e.g. MSI-H or dMMR colorectal cancer), cervical cancer, small cell lung cancer, non-small cell lung cancer, triple negative breast cancer, gastric and esophagogastric junction (GEJ) carcinoma, Hodgkin's lymphoma, Primary mediastinal B-cell lymphoma (PMBCL), and urothelial cancer (e.g. locally advanced or metastatic urothelial cancer).

In some embodiments, the cancer is not responsive to an immunotherapy. For example, in some embodiments, the cancer is not responsive to an immune checkpoint modulator therapy, e.g., an immune checkpoint inhibitor therapy. In some embodiments, the cancer is not responsive to therapy with a PD1 inhibitor, e.g., an anti-PD1 antibody.

In one embodiment, the cancer exhibits reduced RIPK3 expression. A reduction in RIPK3 expression has been reported in several cancers, including colorectal cancer, gastric cancer, ovarian cancer, prostate cancer, adrenocortical cancer and breast cancer. For example, RIPK3 mRNA levels were progressively reduced during tumor growth in colorectal, gastric, and ovarian cancer patients, and the reduction in RIPK3 expression was also associated with the progression to metastasis in human prostate tumors, and higher-grade adrenocortical and breast tumors. See Najafov et al., 2018, PLoS Biol 16(8): e2005756, which is incorporated by reference herein in its entirety. In some embodiments, the cancer exhibits reduced RIPK3 expression relative to a corresponding non-cancerous cell, e.g., an ovarian cancer cell that exhibits reduced RIPK3 expression relative to a non-cancerous ovarian cell. In some embodiments, the cancer exhibits reduced RIPK3 expression relative to a cancer cell of the same type, for example, due to progression of the cancer. In one embodiment, the cancer that exhibits reduced RIPK3 expression is selected from the group consisting of colorectal cancer, gastric cancer, ovarian cancer, prostate cancer, adrenocortical cancer and breast cancer.

In some embodiments, the therapies described herein may be administered to a subject that has previously failed treatment for a cancer with another anti-neoplastic (e.g. immunotherapeutic) regimen. A “subject who has failed an anti-neoplastic regimen” is a subject with cancer that does not respond, or ceases to respond to treatment with an anti-neoplastic regimen per RECIST 1.1 criteria, i.e., does not achieve a complete response, partial response, or stable disease in the target lesion; or does not achieve complete response or non-CR/non-PD of non-target lesions, either during or after completion of the anti-neoplastic regimen, either alone or in conjunction with surgery and/or radiation therapy which, when possible, are often clinically indicated in conjunction with anti-neoplastic therapy. The RECIST 1.1 criteria are described, for example, in Eisenhauer et al., 2009, Eur. J. Cancer 45:228-24 (which is incorporated herein by reference in its entirety), and discussed in greater detail below. A failed anti-neoplastic regimen results in, e.g., tumor growth, increased tumor burden, and/or tumor metastasis. A failed anti-neoplastic regimen as used herein includes a treatment regimen that was terminated due to a dose limiting toxicity, e.g., a grade III or a grade IV toxicity that cannot be resolved to allow continuation or resumption of treatment with the anti-neoplastic agent or regimen that caused the toxicity. In one embodiment, the subject has failed treatment with an anti-neoplastic regimen comprising administration of one or more anti-angiogenic agents.

A failed anti-neoplastic regimen includes a treatment regimen that does not result in at least stable disease for all target and non-target lesions for an extended period, e.g., at least 1 month, at least 2 months, at least 3 months, at least 4 months, at least 5 months, at least 6 months, at least 12 months, at least 18 months, or any time period less than a clinically defined cure. A failed anti-neoplastic regimen includes a treatment regimen that results in progressive disease of at least one target lesion during treatment with the anti-neoplastic agent, or results in progressive disease less than 2 weeks, less than 1 month, less than two months, less than 3 months, less than 4 months, less than 5 months, less than 6 months, less than 12 months, or less than 18 months after the conclusion of the treatment regimen, or less than any time period less than a clinically defined cure.

A failed anti-neoplastic regimen does not include a treatment regimen wherein the subject treated for a cancer achieves a clinically defined cure, e.g., 5 years of complete response after the end of the treatment regimen, and wherein the subject is subsequently diagnosed with a distinct cancer, e.g., more than 5 years, more than 6 years, more than 7 years, more than 8 years, more than 9 years, more than 10 years, more than 11 years, more than 12 years, more than 13 years, more than 14 years, or more than 15 years after the end of the treatment regimen.

RECIST criteria are clinically accepted assessment criteria used to provide a standard approach to solid tumor measurement and provide definitions for objective assessment of change in tumor size for use in clinical trials. Such criteria can also be used to monitor response of an individual undergoing treatment for a solid tumor. The RECIST 1.1 criteria are discussed in detail in Eisenhauer et al., 2009, Eur. J. Cancer 45:228-24, which is incorporated herein by reference. Response criteria for target lesions include:

Complete Response (CR): Disappearance of all target lesions. Any pathological lymph nodes (whether target or non-target) must have a reduction in short axis to <10 mm.

Partial Response (PR): At least a 30% decrease in the sum of diameters of target lesion, taking as a reference the baseline sum diameters.

Progressive Diseases (PD): At least a 20% increase in the sum of diameters of target lesions, taking as a reference the smallest sum on the study (this includes the baseline sum if that is the smallest on the study). In addition to the relative increase of 20%, the sum must also demonstrate an absolute increase of at least 5 mm. (Note: the appearance of one or more new lesions is also considered progression.)

Stable Disease (SD): Neither sufficient shrinkage to qualify for PR nor sufficient increase to qualify for PD, taking as a reference the smallest sum diameters while on study.

RECIST 1.1 criteria also consider non-target lesions which are defined as lesions that may be measureable, but need not be measured, and should only be assessed qualitatively at the desired time points. Response criteria for non-target lesions include:

Complete Response (CR): Disappearance of all non-target lesions and normalization of tumor marker levels. All lymph nodes must be non-pathological in size (<10 mm short axis).

Non-CR/Non-PD: Persistence of one or more non-target lesion(s) and/or maintenance of tumor marker level above the normal limits.

Progressive Disease (PD): Unequivocal progression of existing non-target lesions. The appearance of one or more new lesions is also considered progression. To achieve “unequivocal progression” on the basis of non-target disease, there must be an overall level of substantial worsening of non-target disease such that, even in the presence of SD or PR in target disease, the overall tumor burden has increased sufficiently to merit discontinuation of therapy. A modest “increase” in the size of one or more non-target lesions is usually not sufficient to qualify for unequivocal progression status. The designation of overall progression solely on the basis of change in non-target disease in the face of SD or PR in target disease will therefore be extremely rare.

In some embodiments, the nucleic acid molecules, vectors (e.g. an engineered virus, plasmid or transposon), cells or pharmaceutical compositions and combination therapies described herein may be administered to a subject having a refractory cancer. A “refractory cancer” is a malignancy for which surgery is ineffective, which is either initially unresponsive to chemo- or radiation therapy, or which becomes unresponsive to chemo- or radiation therapy over time.

The invention further provides methods of inhibiting tumor cell growth in a subject, comprising administering a nucleic acid molecule, vector (e.g. an engineered virus, plasmid or transposon), cell or pharmaceutical composition as described herein such that tumor cell growth is inhibited. In certain embodiments, treating cancer comprises extending survival or extending time to tumor progression as compared to a control, e.g. a subject that is not treated with the nucleic acid molecule, vector, cell or pharmaceutical composition described herein. In certain embodiments, the subject is a human subject. In some embodiments, the subject is identified as having cancer (e.g. a tumor) prior to administration of the first dose of the nucleic acid molecule, vector (e.g. an engineered virus, plasmid or transposon), cell or pharmaceutical composition as described herein. In certain embodiments, the subject has cancer (e.g. a tumor) at the time of the first administration of the nucleic acid molecule, vector (e.g. an engineered virus, plasmid or transposon), cell or pharmaceutical composition as described herein.

In one embodiment, administration of the recombinant nucleic acid molecule, vector (e.g. an engineered virus, plasmid or transposon), cell or pharmaceutical composition as described herein results in one or more of, reducing proliferation of cancer cells, reducing metastasis of cancer cells, reducing neovascularization of a tumor, reducing tumor burden, reducing tumor size, weight or volume, inhibiting tumor growth, increased time to progression of the cancer, and/or prolonging the survival time of a subject having an oncological disorder. In certain embodiments, administration of the nucleic acid molecule, vector (e.g. an engineered virus, plasmid or transposon), cell or pharmaceutical composition as described herein reduces proliferation of cancer cells, reduces metastasis of cancer cells, reduces neovascularization of a tumor, reduces tumor burden, reduces tumor size, weight or volume, increases time to progression, inhibits tumor growth and/or prolongs the survival time of the subject by at least 1%, 2%, 3%, 4%, 5%, 10%, 20%, 30%, 40%, 50%, 60%, 70%, 80%, 90%, 100%, 200%, 300%, 400% or 500% relative to a corresponding control subject that is not administered the nucleic acid molecule, vector, cell or pharmaceutical composition. In certain embodiments, administration of the nucleic acid molecule, vector (e.g. an engineered virus, plasmid or transposon), cell or pharmaceutical composition as described herein reduces proliferation of cancer cells, reduces metastasis of cancer cells, reduces neovascularization of a tumor, reduces tumor burden, reduces tumor size, weight or volume, increases time to progression, inhibits tumor growth and/or prolongs the survival time of a population of subjects afflicted with an oncological disorder by at least 1%, 2%, 3%, 4%, 5%, 10%, 20%, 30%, 40%, 50%, 60%, 70%, 80%, 90%, 100%, 200%, 300%, 400% or 500% relative to a corresponding population of control subjects afflicted with the oncological disorder that is not administered the nucleic acid molecule, vector, cell or pharmaceutical composition. In some embodiments, the proliferation of the cancer cells is a hyperproliferation of the cancer cells resulting from a cancer therapy administered to the subject. In some embodiments, administration of the nucleic acid molecule, vector (e.g. an engineered virus, plasmid or transposon), cell or pharmaceutical composition as described herein stabilizes the oncological disorder in a subject with a progressive oncological disorder prior to treatment.

The combination of two or more thanotransmission polypeptides as described herein may improve cancer treatment to a greater extent than each thanotransmission polypeptide alone. For example, in some embodiments, administration of the recombinant nucleic acid molecule encoding two or more different thanotransmission polypeptides to a subject improves cancer treatment (e.g., reduces proliferation of cancer cells, reduces metastasis of cancer cells, reduces neovascularization of a tumor, reduces tumor burden, reduces tumor size, weight or volume, inhibits tumor growth, increases time to progression of the cancer, and/or prolongs the survival time of a subject having the cancer) relative to a subject that is administered a nucleic acid molecule encoding only one of the thanotransmission polypeptides. For example, in some embodiments, administering a recombinant nucleic acid molecule encoding TRIF and RIPK3 improves cancer treatment (e.g., reduces proliferation of cancer cells, reduces metastasis of cancer cells, reduces neovascularization of a tumor, reduces tumor burden, reduces tumor size, weight or volume, inhibits tumor growth, increases time to progression of the cancer, and/or prolongs the survival time of a subject having the cancer) relative to a subject that is administered a recombinant nucleic acid molecule encoding TRIF alone and/or relative to a subject that is administered a recombinant nucleic acid molecule encoding RIPK3 alone. In some embodiments, administering a recombinant nucleic acid molecule encoding TRIF and RIPK3 improves cancer (e.g., colon cancer) treatment (e.g., prolongs survival time) relative to a subject that is administered a recombinant nucleic acid molecule encoding TRIF alone. In some embodiments, administering one or more recombinant nucleic acid molecules encoding TRIF and Gasdermin E improves cancer (e.g., colon cancer) treatment (e.g., prolongs survival time) relative to a subject that is administered a recombinant nucleic acid molecule encoding TRIF alone.

In some embodiments, administering one or more recombinant nucleic acid molecules encoding TRIF and Gasdermin E improves cancer (e.g., colon cancer) treatment (e.g., reduces tumor growth or results in sustained clearance of a tumor) relative to a subject that is not administered a recombinant nucleic acid molecule encoding TRIF or Gasdermin E. In some embodiments, administering one or more recombinant nucleic acid molecules encoding TRIF and Npro improves cancer (e.g., colon cancer) treatment (e.g., reduces tumor growth or results in sustained clearance of a tumor) relative to a subject that is not administered a recombinant nucleic acid molecule encoding TRIF or Npro.

The combination of IL-12 and one or more thanotransmission polypeptides as described herein may improve cancer treatment to a greater extent than IL-12 alone or the one or more thanotransmission polypeptides alone. For example, in some embodiments, administering one or more recombinant nucleic acid molecules encoding IL-12 and Gasdermin E improves cancer (e.g., melanoma or colon cancer) treatment (e.g., reduces tumor growth or results in sustained clearance of a tumor) relative to a subject that is administered a recombinant nucleic acid molecule encoding IL-12 alone or Gasermin E alone. In some embodiments, administering one or more recombinant nucleic acid molecules encoding IL-12 and TRIF improves cancer (e.g., melanoma or colon cancer) treatment (e.g., reduces tumor growth or results in sustained clearance of a tumor) relative to a subject that is administered a recombinant nucleic acid molecule encoding IL-12 alone or TRIF alone.

Addition of a third thanotransmission polypeptide to the two thanotransmission polypeptides may improve cancer treatment relative to the two thanotransmission polypeptides alone. For example, in some embodiments, administration to a subject of a recombinant nucleic acid molecule encoding two different thanotransmission polypeptides as described herein and further encoding a third thanotransmission polypeptide improves cancer treatment (e.g., reduces proliferation of cancer cells, reduces metastasis of cancer cells, reduces neovascularization of a tumor, reduces tumor burden, reduces tumor size, weight or volume, inhibits tumor growth, increases time to progression of the cancer, and/or prolongs the survival time of a subject having the cancer) relative to a subject that is administered a nucleic acid molecule that encodes the two different thanotransmission polypeptides, but does not further encode the third thanotransmission polypeptide. For example, in some embodiments, administration of a recombinant nucleic acid molecule encoding TRIF, RIPK3 and vICA improves cancer treatment (e.g., reduces proliferation of cancer cells, reduces metastasis of cancer cells, reduces neovascularization of a tumor, reduces tumor burden, reduces tumor size, weight or volume, inhibits tumor growth, increases time to progression of the cancer, and/or prolongs the survival time of a subject having the cancer) relative to a subject that is administered a nucleic acid molecule that encodes TRIF and RIPK3, but does not vICA. In some embodiments, administration of a recombinant nucleic acid molecule encoding TRIF, RIPK3 and vICA improves cancer (e.g., colon cancer) treatment (e.g., reduces tumor growth) relative to a subject that is administered a nucleic acid molecule that encodes TRIF and RIPK3, but does not encode vICA.

Inclusion of Gasdermin E in the two or more thanotransmission polyeptides may improve cancer treatment relative to one or more thanotransmission polyeptides that do not include Gasdermin E. For example, in some embodiments, administration of a recombinant nucleic acid molecule encoding Gasdermin E and one or more additional thanotransmission polypeptide (e.g., a thanotransmission polypeptide selected from TRIF, RIPK3, vICA, Npro, A238L, vMLKL, and variants thereof, or a dominant negative variant of IKBa) improves cancer treatment (e.g., reduces proliferation of cancer cells, reduces metastasis of cancer cells, reduces neovascularization of a tumor, reduces tumor burden, reduces tumor size, weight or volume, inhibits tumor growth, increases time to progression of the cancer, and/or prolongs the survival time of a subject having the cancer) relative to a nucleic acid molecule encoding the one or more additional thanotransmission polypeptides but not encoding Gasdermin E. In some embodiments, administration of a recombinant nucleic acid molecule encoding TRIF and Gasdermin E improves cancer treatment (e.g., reduces proliferation of cancer cells, reduces metastasis of cancer cells, reduces neovascularization of a tumor, reduces tumor burden, reduces tumor size, weight or volume, inhibits tumor growth, increases time to progression of the cancer, and/or prolongs the survival time of a subject having the cancer) relative to a nucleic acid molecule encoding TRIF alone. In some embodiments, administration of a recombinant nucleic acid molecule encoding TRIF, RIPK3 and Gasdermin E improves cancer treatment (e.g., reduces proliferation of cancer cells, reduces metastasis of cancer cells, reduces neovascularization of a tumor, reduces tumor burden, reduces tumor size, weight or volume, inhibits tumor growth, increases time to progression of the cancer, and/or prolongs the survival time of a subject having the cancer) relative to a nucleic acid molecule encoding TRIF and RIPK3 alone. In some embodiments, administration of a recombinant nucleic acid molecule encoding TRIF and Gasdermin E improves cancer (e.g., colon cancer) treatment (e.g., prolongs survival time) relative to administration of a recombinant nucleic acid molecule encoding TRIF alone. In some embodiments, administration of a recombinant nucleic acid molecule encoding TRIF, RIPK3 and Gasdermin E improves cancer (e.g., colon cancer) treatment (e.g., prolongs survival time) relative to administration of a recombinant nucleic acid molecule encoding TRIF and RIPK3 alone.

Use of IL-12 with one or more additional thanotransmission polyeptides may improve cancer treatment relative to IL-12 alone. For example, in some embodiments, administration of one or more recombinant nucleic acid molecules encoding IL-12 and one or more additional thanotransmission polypeptides (e.g., a thanotransmission polypeptide selected from TRIF, RIPK3, vICA, Npro, A238L, vMLKL, and variants thereof, or a dominant negative variant of IKBa) improves cancer treatment (e.g., reduces proliferation of cancer cells, reduces metastasis of cancer cells, reduces neovascularization of a tumor, reduces tumor burden, reduces tumor size, weight or volume, inhibits tumor growth, increases time to progression of the cancer, and/or prolongs the survival time of a subject having the cancer) relative to administration of one or more nucleic acid molecules encoding IL-12 alone. In some embodiments, administration of one or more recombinant nucleic acid molecules encoding IL-12 and TRIF improves cancer treatment (e.g., reduces proliferation of cancer cells, reduces metastasis of cancer cells, reduces neovascularization of a tumor, reduces tumor burden, reduces tumor size, weight or volume, inhibits tumor growth, increases time to progression of the cancer, and/or prolongs the survival time of a subject having the cancer) relative to one or more nucleic acid molecules encoding IL-12 alone. In some embodiments, administration of one or more recombinant nucleic acid molecules encoding IL-12 and Gasdermin E improves cancer treatment (e.g., reduces proliferation of cancer cells, reduces metastasis of cancer cells, reduces neovascularization of a tumor, reduces tumor burden, reduces tumor size, weight or volume, inhibits tumor growth, increases time to progression of the cancer, and/or prolongs the survival time of a subject having the cancer) relative to one or more nucleic acid molecules encoding IL-12 alone.

In some embodiments, administration of one or more recombinant nucleic acid molecules encoding IL-12, TRIF, and Gasdermin E improves cancer treatment (e.g., reduces proliferation of cancer cells, reduces metastasis of cancer cells, reduces neovascularization of a tumor, reduces tumor burden, reduces tumor size, weight or volume, inhibits tumor growth, increases time to progression of the cancer, and/or prolongs the survival time of a subject having the cancer) relative to one or more nucleic acid molecules encoding IL-12 alone, or relative to one or more nucleic acid molecules encoding TRIF and Gasdermin E alone. In some embodiments, administration of one or more recombinant nucleic acid molecules encoding IL-12, TRIF and Gasdermin E reduces tumor growth (e.g. melanoma tumor growth) relative to one or more recombinant nucleic acid molecules encoding IL-12 alone, or relative to one or more recombinant nucleic acid molecules encoding TRIF and Gasdermin E alone.

Combination Therapy of a Nucleic Acid Molecule, Vector (e.g., an Engineered Virus, Plasmid or Transposon), Cell or Pharmaceutical Composition as Described Herein, and Additional Therapeutic Agents.

The terms “administering in combination”, “combination therapy”, “co-administering” or “co-administration” may refer to administration of the nucleic acid molecule, vector (e.g., an engineered virus, plasmid or transposon), cell or pharmaceutical composition as described herein in combination with one or more additional therapeutic agents. The one or more additional therapeutic agents may be administered prior to, concurrently or substantially concurrently with, subsequently to, or intermittently with administration of the nucleic acid molecule, vector (e.g., an engineered virus, plasmid or transposon), cell or pharmaceutical composition as described herein. In certain embodiments, the one or more additional therapeutic agents is administered prior to administration of the nucleic acid molecule, vector (e.g., an engineered virus, plasmid or transposon), cell or pharmaceutical composition as described herein. In certain embodiments, the one or more additional therapeutic agents is administered concurrently with the nucleic acid molecule, vector (e.g., an engineered virus, plasmid or transposon), cell or pharmaceutical composition as described herein. In certain embodiments, the one or more additional therapeutic agents is administered after administration of the recombinant nucleic acid molecule, vector (e.g. an engineered virus, plasmid or transposon), cell or pharmaceutical composition as described herein.

The one or more additional therapeutic agents and the recombinant nucleic acid molecule, vector (e.g. an engineered virus, plasmid or transposon), cell or pharmaceutical composition as described herein act additively or synergistically. In one embodiment, the one or more additional therapeutic agents and the recombinant nucleic acid molecule, vector (e.g. an engineered virus, plasmid or transposon), cell or pharmaceutical composition as described herein act synergistically. In some embodiments the synergistic effects are in the treatment of an oncological disorder (e.g., reduction in tumor growth) or an infection. For example, in one embodiment, the combination of the one or more additional therapeutic agents and the recombinant nucleic acid molecule, vector (e.g. an engineered virus, plasmid or transposon), cell or pharmaceutical composition as described herein improves the durability, i.e. extends the duration, of the immune response against a cancer. In some embodiments, the one or more additional therapeutic agents and the recombinant nucleic acid molecule, vector (e.g. an engineered virus, plasmid or transposon), cell or pharmaceutical composition as described herein act additively.

### 1. Immune Checkpoint Modulators

In some embodiments, the additional therapeutic agent administered in combination with the recombinant nucleic acid molecule, vector (e.g. an engineered virus, plasmid or transposon), cell or pharmaceutical composition as described herein is an immune checkpoint modulator of an immune checkpoint molecule. Examples of immune checkpoint molecules include LAG-3 (Triebel et al., 1990, J. Exp. Med. 171: 1393-1405), TIM-3 (Sakuishi et al., 2010, J. Exp. Med. 207: 2187-2194), VISTA (Wang et al., 2011, J. Exp. Med. 208: 577-592), ICOS (Fan et al., 2014, J. Exp. Med. 211: 715-725), OX40 (Curti et al., 2013, Cancer Res. 73: 7189-7198) and 4-1BB (Melero et al., 1997, Nat. Med. 3: 682-685).

Immune checkpoints may be stimulatory immune checkpoints (i.e. molecules that stimulate the immune response) or inhibitory immune checkpoints (i.e. molecules that inhibit immune response). In some embodiments, the immune checkpoint modulator is an antagonist of an inhibitory immune checkpoint. In some embodiments, the immune checkpoint modulator is an agonist of a stimulatory immune checkpoint. In some embodiments, the immune checkpoint modulator is an immune checkpoint binding protein (e.g., an antibody, antibody Fab fragment, divalent antibody, antibody drug conjugate, scFv, fusion protein, bivalent antibody, or tetravalent antibody). In certain embodiments, the immune checkpoint modulator is capable of binding to, or modulating the activity of more than one immune checkpoint. Examples of stimulatory and inhibitory immune checkpoints, and molecules that modulate these immune checkpoints that may be used in the methods of the invention, are provided below.

i. Stimulatory Immune Checkpoint Molecules

CD27 supports antigen-specific expansion of naïve T cells and is vital for the generation of T cell memory (see, e.g., Hendriks et al. (2000) Nat. Immunol. 171 (5): 433-40). CD27 is also a memory marker of B cells (see, e.g., Agematsu et al. (2000) Histol. Histopathol. 15 (2): 573-6. CD27 activity is governed by the transient availability of its ligand, CD70, on lymphocytes and dendritic cells (see, e.g., Borst et al. (2005) Curr. Opin. Immunol. 17 (3): 275-81). Multiple immune checkpoint modulators specific for CD27 have been developed and may be used as disclosed herein. In some embodiments, the immune checkpoint modulator is an agent that modulates the activity and/or expression of CD27. In some embodiments, the immune checkpoint modulator is an agent that binds to CD27 (e.g., an anti-CD27 antibody). In some embodiments, the checkpoint modulator is a CD27 agonist. In some embodiments, the checkpoint modulator is a CD27 antagonist. In some embodiments, the immune checkpoint modulator is an CD27-binding protein (e.g., an antibody). In some embodiments, the immune checkpoint modulator is varlilumab (Celldex Therapeutics). Additional CD27-binding proteins (e.g., antibodies) are known in the art and are disclosed, e.g., in U.S. Pat. Nos. 9,248,183, 9,102,737, 9,169,325, 9,023,999, 8,481,029; U.S. Patent Application Publication Nos. 2016/0185870, 2015/0337047, 2015/0299330, 2014/0112942, 2013/0336976, 2013/0243795, 2013/0183316, 2012/0213771, 2012/0093805, 2011/0274685, 2010/0173324; and PCT Publication Nos. WO 2015/016718, WO 2014/140374, WO 2013/138586, WO 2012/004367, WO 2011/130434, WO 2010/001908, and WO 2008/051424, each of which is incorporated by reference herein.

CD28. Cluster of Differentiation 28 (CD28) is one of the proteins expressed on T cells that provide co-stimulatory signals required for T cell activation and survival. T cell stimulation through CD28 in addition to the T-cell receptor (TCR) can provide a potent signal for the production of various interleukins (IL-6 in particular). Binding with its two ligands, CD80 and CD86, expressed on dendritic cells, prompts T cell expansion (see, e.g., Prasad et al. (1994) Proc. Nat'l. Acad. Sci. USA 91(7): 2834-8). Multiple immune checkpoint modulators specific for CD28 have been developed and may be used as disclosed herein. In some embodiments, the immune checkpoint modulator is an agent that modulates the activity and/or expression of CD28. In some embodiments, the immune checkpoint modulator is an agent that binds to CD28 (e.g., an anti-CD28 antibody). In some embodiments, the checkpoint modulator is an CD28 agonist. In some embodiments, the checkpoint modulator is an CD28 antagonist. In some embodiments, the immune checkpoint modulator is an CD28-binding protein (e.g., an antibody). In some embodiments, the immune checkpoint modulator is selected from the group consisting of TAB08 (TheraMab LLC), lulizumab (also known as BMS-931699, Bristol-Myers Squibb), and FR104 (OSE Immunotherapeutics). Additional CD28-binding proteins (e.g., antibodies) are known in the art and are disclosed, e.g., in U.S. Pat. Nos. 9,119,840, 8,709,414, 9,085,629, 8,034,585, 7,939,638, 8,389,016, 7,585,960, 8,454,959, 8,168,759, 8,785,604, 7,723,482; U.S. Patent Application Publication Nos. 2016/0017039, 2015/0299321, 2015/0150968, 2015/0071916, 2015/0376278, 2013/0078257, 2013/0230540, 2013/0078236, 2013/0109846, 2013/0266577, 2012/0201814, 2012/0082683, 2012/0219553, 2011/0189735, 2011/0097339, 2010/0266605, 2010/0168400, 2009/0246204, 2008/0038273; and PCT Publication Nos. WO 2015198147, WO 2016/05421, WO 2014/1209168, WO 2011/101791, WO 2010/007376, WO 2010/009391, WO 2004/004768, WO 2002/030459, WO 2002/051871, and WO 2002/047721, each of which is incorporated by reference herein.

CD40. Cluster of Differentiation 40 (CD40, also known as TNFRSF5) is found on a variety of immune system cells including antigen presenting cells. CD40L, otherwise known as CD154, is the ligand of CD40 and is transiently expressed on the surface of activated CD4⁺ T cells. CD40 signaling is known to ‘license’ dendritic cells to mature and thereby trigger T-cell activation and differentiation (see, e.g., O'Sullivan et al. (2003) Crit. Rev. Immunol. 23 (1): 83-107. Multiple immune checkpoint modulators specific for CD40 have been developed and may be used as disclosed herein. In some embodiments, the immune checkpoint modulator is an agent that modulates the activity and/or expression of CD40. In some embodiments, the immune checkpoint modulator is an agent that binds to CD40 (e.g., an anti-CD40 antibody). In some embodiments, the checkpoint modulator is a CD40 agonist.

In some embodiments, the checkpoint modulator is an CD40 antagonist. In some embodiments, the immune checkpoint modulator is a CD40-binding protein selected from the group consisting of dacetuzumab (Genentech/Seattle Genetics), CP-870,893 (Pfizer), bleselumab (Astellas Pharma), lucatumumab (Novartis), CFZ533 (Novartis; see, e.g., Cordoba et al. (2015) Am. J. Transplant. 15(11): 2825-36), RG7876 (Genentech Inc.), FFP104 (PanGenetics, B.V.), APX005 (Apexigen), BI 655064 (Boehringer Ingelheim), Chi Lob 7/4 (Cancer Research UK; see, e.g., Johnson et al. (2015) Clin. Cancer Res. 21(6): 1321-8), ADC-1013 (BioInvent International), SEA-CD40 (Seattle Genetics), XmAb 5485 (Xencor), PG120 (PanGenetics B.V.), teneliximab (Bristol-Myers Squibb; see, e.g., Thompson et al. (2011)Am. J Transplant. 11(5): 947-57), and AKH3 (Biogen; see, e.g., International Publication No. WO 2016/028810). Additional CD40-binding proteins (e.g., antibodies) are known in the art and are disclosed, e.g., in U.S. Pat. Nos. 9,234,044, 9,266,956, 9,109,011, 9,090,696, 9,023,360, 9,023,361, 9,221,913, 8,945,564, 8,926,979, 8,828,396, 8,637,032, 8,277,810, 8,088,383, 7,820,170, 7,790,166, 7,445,780, 7,361,345, 8,961,991, 8,669,352, 8,957,193, 8,778,345, 8,591,900, 8,551,485, 8,492,531, 8,362,210, 8,388,971; U.S. Patent Application Publication Nos. 2016/0045597, 2016/0152713, 2016/0075792, 2015/0299329, 2015/0057437 2015/0315282, 2015/0307616, 2014/0099317, 2014/0179907, 2014/0349395, 2014/0234344, 2014/0348836, 2014/0193405, 2014/0120103, 2014/0105907, 2014/0248266, 2014/0093497, 2014/0010812, 2013/0024956, 2013/0023047, 2013/0315900, 2012/0087927, 2012/0263732, 2012/0301488, 2011/0027276, 2011/0104182, 2010/0234578, 2009/0304687, 2009/0181015, 2009/0130715, 2009/0311254, 2008/0199471, 2008/0085531, 2016/0152721, 2015/0110783, 2015/0086991, 2015/0086559, 2014/0341898, 2014/0205602, 2014/0004131, 2013/0011405, 2012/0121585, 2011/0033456, 2011/0002934, 2010/0172912, 2009/0081242, 2009/0130095, 2008/0254026, 2008/0075727, 2009/0304706, 2009/0202531, 2009/0117111, 2009/0041773, 2008/0274118, 2008/0057070, 2007/0098717, 2007/0218060, 2007/0098718, 2007/0110754; and PCT Publication Nos. WO 2016/069919, WO 2016/023960, WO 2016/023875, WO 2016/028810, WO 2015/134988, WO 2015/091853, WO 2015/091655, WO 2014/065403, WO 2014/070934, WO 2014/065402, WO 2014/207064, WO 2013/034904, WO 2012/125569, WO 2012/149356, WO 2012/111762, WO 2012/145673, WO 2011/123489, WO 2010/123012, WO 2010/104761, WO 2009/094391, WO 2008/091954, WO 2007/129895, WO 2006/128103, WO 2005/063289, WO 2005/063981, WO 2003/040170, WO 2002/011763, WO 2000/075348, WO 2013/164789, WO 2012/075111, WO 2012/065950, WO 2009/062054, WO 2007/124299, WO 2007/053661, WO 2007/053767, WO 2005/044294, WO 2005/044304, WO 2005/044306, WO 2005/044855, WO 2005/044854, WO 2005/044305, WO 2003/045978, WO 2003/029296, WO 2002/028481, WO 2002/028480, WO 2002/028904, WO 2002/028905, WO 2002/088186, and WO 2001/024823, each of which is incorporated by reference herein.

CD122. CD122 is the Interleukin-2 receptor beta sub-unit and is known to increase proliferation of CD8⁺ effector T cells. See, e.g., Boyman et al. (2012) Nat. Rev. Immunol. 12 (3): 180-190. Multiple immune checkpoint modulators specific for CD122 have been developed and may be used as disclosed herein. In some embodiments, the immune checkpoint modulator is an agent that modulates the activity and/or expression of CD122. In some embodiments, the immune checkpoint modulator is an agent that binds to CD122 (e.g., an anti-CD122 antibody). In some embodiments, the checkpoint modulator is an CD122 agonist. In some embodiments, the checkpoint modulator is an CD22 agonist. In some embodiments, the immune checkpoint modulator is humanized MiK-Beta-1 (Roche; see, e.g., Morris et al. (2006) Proc Nat'l. Acad. Sci. USA 103(2): 401-6, which is incorporated by reference). Additional CD122-binding proteins (e.g., antibodies) are known in the art and are disclosed, e.g., in U.S. Pat. No. 9,028,830, which is incorporated by reference herein.

OX40. The OX40 receptor (also known as CD134) promotes the expansion of effector and memory T cells. OX40 also suppresses the differentiation and activity of T-regulatory cells, and regulates cytokine production (see, e.g., Croft et al. (2009) Immunol. Rev. 229(1): 173-91). Multiple immune checkpoint modulators specific for OX40 have been developed and may be used as disclosed herein. In some embodiments, the immune checkpoint modulator is an agent that modulates the activity and/or expression of OX40. In some embodiments, the immune checkpoint modulator is an agent that binds to OX40 (e.g., an anti-OX40 antibody). In some embodiments, the checkpoint modulator is an OX40 agonist. In some embodiments, the checkpoint modulator is an OX40 antagonist. In some embodiments, the immune checkpoint modulator is a OX40-binding protein (e.g., an antibody) selected from the group consisting of MEDI6469 (AgonOx/Medimmune), pogalizumab (also known as MOXR0916 and RG7888; Genentech, Inc.), tavolixizumab (also known as MEDI0562; Medimmune), and GSK3174998 (GlaxoSmithKline). Additional OX-40-binding proteins (e.g., antibodies) are known in the art and are disclosed, e.g, in U.S. Pat. Nos. 9,163,085, 9,040,048, 9,006,396, 8,748,585, 8,614,295, 8,551,477, 8,283,450, 7,550,140; U.S. Patent Application Publication Nos. 2016/0068604, 2016/0031974, 2015/0315281, 2015/0132288, 2014/0308276, 2014/0377284, 2014/0044703, 2014/0294824, 2013/0330344, 2013/0280275, 2013/0243772, 2013/0183315, 2012/0269825, 2012/0244076, 2011/0008368, 2011/0123552, 2010/0254978, 2010/0196359, 2006/0281072; and PCT Publication Nos. WO 2014/148895, WO 2013/068563, WO 2013/038191, WO 2013/028231, WO 2010/096418, WO 2007/062245, and WO 2003/106498, each of which is incorporated by reference herein.

GITR. Glucocorticoid-induced TNFR family related gene (GITR) is a member of the tumor necrosis factor receptor (TNFR) superfamily that is constitutively or conditionally expressed on Treg, CD4, and CD8 T cells. GITR is rapidly upregulated on effector T cells following TCR ligation and activation. The human GITR ligand (GITRL) is constitutively expressed on APCs in secondary lymphoid organs and some nonlymphoid tissues. The downstream effect of GITR:GITRL interaction induces attenuation of Treg activity and enhances CD4⁺ T cell activity, resulting in a reversal of Treg-mediated immunosuppression and increased immune stimulation. Multiple immune checkpoint modulators specific for GITR have been developed and may be used as disclosed herein. In some embodiments, the immune checkpoint modulator is an agent that modulates the activity and/or expression of GITR. In some embodiments, the immune checkpoint modulator is an agent that binds to GITR (e.g., an anti-GITR antibody). In some embodiments, the checkpoint modulator is an GITR agonist. In some embodiments, the checkpoint modulator is an GITR antagonist. In some embodiments, the immune checkpoint modulator is a GITR-binding protein (e.g., an antibody) selected from the group consisting of TRX518 (Leap Therapeutics), MK-4166 (Merck & Co.), MEDI-1873 (MedImmune), INCAGN1876 (Agenus/Incyte), and FPA154 (Five Prime Therapeutics). Additional GITR-binding proteins (e.g., antibodies) are known in the art and are disclosed, e.g., in U.S. Pat. Nos. 9,309,321, 9,255,152, 9,255,151, 9,228,016, 9,028,823, 8,709,424, 8,388,967; U.S. Patent Application Publication Nos. 2016/0145342, 2015/0353637, 2015/0064204, 2014/0348841, 2014/0065152, 2014/0072566, 2014/0072565, 2013/0183321, 2013/0108641, 2012/0189639; and PCT Publication Nos. WO 2016/054638, WO 2016/057841, WO 2016/057846, WO 2015/187835, WO 2015/184099, WO 2015/031667, WO 2011/028683, and WO 2004/107618, each of which is incorporated by reference herein.

ICOS. Inducible T-cell costimulator (ICOS, also known as CD278) is expressed on activated T cells. Its ligand is ICOSL, which is expressed mainly on B cells and dendritic cells. ICOS is important in T cell effector function. ICOS expression is up-regulated upon T cell activation (see, e.g., Fan et al. (2014) J. Exp. Med. 211(4): 715-25). Multiple immune checkpoint modulators specific for ICOS have been developed and may be used as disclosed herein. In some embodiments, the immune checkpoint modulator is an agent that modulates the activity and/or expression of ICOS. In some embodiments, the immune checkpoint modulator is an agent that binds to ICOS (e.g., an anti-ICOS antibody). In some embodiments, the checkpoint modulator is an ICOS agonist. In some embodiments, the checkpoint modulator is an ICOS antagonist. In some embodiments, the immune checkpoint modulator is a ICOS-binding protein (e.g., an antibody) selected from the group consisting of MEDI-570 (also known as JMab-136, Medimmune), GSK3359609 (GlaxoSmithKline/INSERM), and JTX-2011 (Jounce Therapeutics). Additional ICOS-binding proteins (e.g., antibodies) are known in the art and are disclosed, e.g., in U.S. Pat. Nos. 9,376,493, 7,998,478, 7,465,445, 7,465,444; U.S. Patent Application Publication Nos. 2015/0239978, 2012/0039874, 2008/0199466, 2008/0279851; and PCT Publication No. WO 2001/087981, each of which is incorporated by reference herein.

4-1BB. 4-1BB (also known as CD137) is a member of the tumor necrosis factor (TNF) receptor superfamily. 4-1BB (CD137) is a type II transmembrane glycoprotein that is inducibly expressed on primed CD4⁺ and CD8⁺ T cells, activated NK cells, DCs, and neutrophils, and acts as a T cell costimulatory molecule when bound to the 4-1BB ligand (4-1BBL) found on activated macrophages, B cells, and DCs. Ligation of the 4-1BB receptor leads to activation of the NF-κB, c-Jun and p38 signaling pathways and has been shown to promote survival of CD8⁺ T cells, specifically, by upregulating expression of the antiapoptotic genes BcL-x(L) and Bfl-1. In this manner, 4-1BB serves to boost or even salvage a suboptimal immune response. Multiple immune checkpoint modulators specific for 4-1BB have been developed and may be used as disclosed herein. In some embodiments, the immune checkpoint modulator is an agent that modulates the activity and/or expression of 4-1BB. In some embodiments, the immune checkpoint modulator is an agent that binds to 4-1BB (e.g., an anti-4-1BB antibody). In some embodiments, the checkpoint modulator is an 4-1BB agonist. In some embodiments, the checkpoint modulator is an 4-1BB antagonist. In some embodiments, the immune checkpoint modulator is a 4-1BB-binding protein is urelumab (also known as BMS-663513; Bristol-Myers Squibb) or utomilumab (Pfizer). In some embodiments, the immune checkpoint modulator is a 4-1BB-binding protein (e.g., an antibody). 4-1BB-binding proteins (e.g., antibodies) are known in the art and are disclosed, e.g., in U.S. Pat. Nos. 9,382,328, 8,716,452, 8,475,790, 8,137,667, 7,829,088, 7,659,384; U.S. Patent Application Publication Nos. 2016/0083474, 2016/0152722, 2014/0193422, 2014/0178368, 2013/0149301, 2012/0237498, 2012/0141494, 2012/0076722, 2011/0177104, 2011/0189189, 2010/0183621, 2009/0068192, 2009/0041763, 2008/0305113, 2008/0008716; and PCT Publication Nos. WO 2016/029073, WO 2015/188047, WO 2015/179236, WO 2015/119923, WO 2012/032433, WO 2012/145183, WO 2011/031063, WO 2010/132389, WO 2010/042433, WO 2006/126835, WO 2005/035584, WO 2004/010947; and Martinez-Forero et al. (2013) J. Immunol. 190(12): 6694-706, and Dubrot et al. (2010) Cancer Immunol. Immunother. 59(8): 1223-33, each of which is incorporated by reference herein.

ii. Inhibitory Immune Checkpoint Molecules

ADORA2A. The adenosine A2A receptor (A2A4) is a member of the G protein-coupled receptor (GPCR) family which possess seven transmembrane alpha helices, and is regarded as an important checkpoint in cancer therapy. A2A receptor can negatively regulate overreactive immune cells (see, e.g., Ohta et al. (2001) Nature 414(6866): 916-20). Multiple immune checkpoint modulators specific for ADORA2A have been developed and may be used as disclosed herein. In some embodiments, the immune checkpoint modulator is an agent that modulates the activity and/or expression of ADORA2A. In some embodiments, the immune checkpoint modulator is an agent that binds to ADORA2A (e.g., an anti-ADORA2A antibody). In some embodiments, the immune checkpoint modulator is a ADORA2A-binding protein (e.g., an antibody). In some embodiments, the checkpoint modulator is an ADORA2A agonist. In some embodiments, the checkpoint modulator is an ADORA2A antagonist. ADORA2A-binding proteins (e.g., antibodies) are known in the art and are disclosed, e.g., in U.S. Patent Application Publication No. 2014/0322236, which is incorporated by reference herein.

B7-H3. B7-H3 (also known as CD276) belongs to the B7 superfamily, a group of molecules that costimulate or down-modulate T-cell responses. B7-H3 potently and consistently down-modulates human T-cell responses (see, e.g., Leitner et al. (2009) Eur. J. Immunol. 39(7): 1754-64). Multiple immune checkpoint modulators specific for B7-H3 have been developed and may be used as disclosed herein. In some embodiments, the immune checkpoint modulator is an agent that modulates the activity and/or expression of B7-H3. In some embodiments, the immune checkpoint modulator is an agent that binds to B7-H3 (e.g., an anti-B7-H3 antibody). In some embodiments, the checkpoint modulator is an B7-H3 agonist. In some embodiments, the checkpoint modulator is an B7-H3 antagonist. In some embodiments, the immune checkpoint modulator is an anti-B7-H3-binding protein selected from the group consisting of DS-5573 (Daiichi Sankyo, Inc.), enoblituzumab (MacroGenics, Inc.), and 8H9 (Sloan Kettering Institute for Cancer Research; see, e.g., Ahmed et al. (2015) J. Biol. Chem. 290(50): 30018-29). In some embodiments, the immune checkpoint modulator is a B7-H3-binding protein (e.g., an antibody). B7-H3-binding proteins (e.g., antibodies) are known in the art and are disclosed, e.g., in U.S. Pat. Nos. 9,371,395, 9,150,656, 9,062,110, 8,802,091, 8,501,471, 8,414,892; U.S. Patent Application Publication Nos. 2015/0352224, 2015/0297748, 2015/0259434, 2015/0274838, 2014/032875, 2014/0161814, 2013/0287798, 2013/0078234, 2013/0149236, 2012/02947960, 2010/0143245, 2002/0102264; PCT Publication Nos. WO 2016/106004, WO 2016/033225, WO 2015/181267, WO 2014/057687, WO 2012/147713, WO 2011/109400, WO 2008/116219, WO 2003/075846, WO 2002/032375; and Shi et al. (2016) Mol. Med. Rep. 14(1): 943-8, each of which is incorporated by reference herein.

B7-H4. B7-H4 (also known as 08E, OV064, and V-set domain-containing T-cell activation inhibitor (VTCN1)), belongs to the B7 superfamily. By arresting cell cycle, B7-H4 ligation of T cells has a profound inhibitory effect on the growth, cytokine secretion, and development of cytotoxicity. Administration of B7-H4Ig into mice impairs antigen-specific T cell responses, whereas blockade of endogenous B7-H4 by specific monoclonal antibody promotes T cell responses (see, e.g., Sica et al. (2003) Immunity 18(6): 849-61). Multiple immune checkpoint modulators specific for B7-H4 have been developed and may be used as disclosed herein. In some embodiments, the immune checkpoint modulator is an agent that modulates the activity and/or expression of B7-H4. In some embodiments, the immune checkpoint modulator is an agent that binds to B7-H4 (e.g., an anti-B7-H4 antibody). In some embodiments, the immune checkpoint modulator is a B7-H4-binding protein (e.g., an antibody). In some embodiments, the checkpoint modulator is an B7-H4 agonist. In some embodiments, the checkpoint modulator is an B7-H4 antagonist. B7-H4-binding proteins (e.g., antibodies) are known in the art and are disclosed, e.g., in U.S. Pat. Nos. 9,296,822, 8,609,816, 8,759,490, 8,323,645; U.S. Patent Application Publication Nos. 2016/0159910, 2016/0017040, 2016/0168249, 2015/0315275, 2014/0134180, 2014/0322129, 2014/0356364, 2014/0328751, 2014/0294861, 2014/0308259, 2013/0058864, 2011/0085970, 2009/0074660, 2009/0208489; and PCT Publication Nos. WO 2016/040724, WO 2016/070001, WO 2014/159835, WO 2014/100483, WO 2014/100439, WO 2013/067492, WO 2013/025779, WO 2009/073533, WO 2007/067991, and WO 2006/104677, each of which is incorporated by reference herein.

BTLA. B and T Lymphocyte Attenuator (BTLA), also known as CD272, has HVEM (Herpesvirus Entry Mediator) as its ligand. Surface expression of BTLA is gradually downregulated during differentiation of human CD8⁺ T cells from the naive to effector cell phenotype, however tumor-specific human CD8⁺ T cells express high levels of BTLA (see, e.g., Derre et al. (2010) J. Clin. Invest. 120 (1): 157-67). Multiple immune checkpoint modulators specific for BTLA have been developed and may be used as disclosed herein. In some embodiments, the immune checkpoint modulator is an agent that modulates the activity and/or expression of BTLA. In some embodiments, the immune checkpoint modulator is an agent that binds to BTLA (e.g., an anti-BTLA antibody). In some embodiments, the immune checkpoint modulator is a BTLA-binding protein (e.g., an antibody). In some embodiments, the checkpoint modulator is an BTLA agonist. In some embodiments, the checkpoint modulator is an BTLA antagonist. BTLA-binding proteins (e.g., antibodies) are known in the art and are disclosed, e.g., in U.S. Pat. Nos. 9,346,882, 8,580,259, 8,563,694, 8,247,537; U.S. Patent Application Publication Nos. 2014/0017255, 2012/0288500, 2012/0183565, 2010/0172900; and PCT Publication Nos. WO 2011/014438, and WO 2008/076560, each of which is incorporated by reference herein.

CTLA-4. Cytotoxic T lymphocyte antigen-4 (CTLA-4) is a member of the immune regulatory CD28-B7 immunoglobulin superfamily and acts on naïve and resting T lymphocytes to promote immunosuppression through both B7-dependent and B7-independent pathways (see, e.g., Kim et al. (2016) J. Immunol. Res., Article ID 4683607, 14 pp.). CTLA-4 is also known as called CD152. CTLA-4 modulates the threshold for T cell activation. See, e.g., Gajewski et al. (2001) J. Immunol. 166(6): 3900-7. Multiple immune checkpoint modulators specific for CTLA-4 have been developed and may be used as disclosed herein. In some embodiments, the immune checkpoint modulator is an agent that modulates the activity and/or expression of CTLA-4. In some embodiments, the immune checkpoint modulator is an agent that binds to CTLA-4 (e.g., an anti-CTLA-4 antibody). In some embodiments, the checkpoint modulator is an CTLA-4 agonist. In some embodiments, the checkpoint modulator is an CTLA-4 antagonist. In some embodiments, the immune checkpoint modulator is a CTLA-4-binding protein (e.g., an antibody) selected from the group consisting of ipilimumab (Yervoy; Medarex/Bristol-Myers Squibb), tremelimumab (formerly ticilimumab; Pfizer/AstraZeneca), JMW-3B3 (University of Aberdeen), and AGEN1884 (Agenus). Additional CTLA-4 binding proteins (e.g., antibodies) are known in the art and are disclosed, e.g., in U.S. Pat. No. 8,697,845; U.S. Patent Application Publication Nos. 2014/0105914, 2013/0267688, 2012/0107320, 2009/0123477; and PCT Publication Nos. WO 2014/207064, WO 2012/120125, WO 2016/015675, WO 2010/097597, WO 2006/066568, and WO 2001/054732, each of which is incorporated by reference herein.

IDO. Indoleamine 2,3-dioxygenase (IDO) is a tryptophan catabolic enzyme with immune-inhibitory properties. Another important molecule is TDO, tryptophan 2,3-dioxygenase. IDO is known to suppress T and NK cells, generate and activate Tregs and myeloid-derived suppressor cells, and promote tumor angiogenesis. Prendergast et al., 2014, Cancer Immunol Immunother. 63 (7): 721-35, which is incorporated by reference herein.

Multiple immune checkpoint modulators specific for IDO have been developed and may be used as disclosed herein. In some embodiments, the immune checkpoint modulator is an agent that modulates the activity and/or expression of IDO. In some embodiments, the immune checkpoint modulator is an agent that binds to IDO (e.g., an IDO binding protein, such as an anti-IDO antibody). In some embodiments, the checkpoint modulator is an IDO agonist. In some embodiments, the checkpoint modulator is an IDO antagonist. In some embodiments, the immune checkpoint modulator is selected from the group consisting of Norharmane, Rosmarinic acid, COX-2 inhibitors, alpha-methyl-tryptophan, and Epacadostat. In one embodiment, the modulator is Epacadostat.

KIR. Killer immunoglobulin-like receptors (KIRs) comprise a diverse repertoire of MHCI binding molecules that negatively regulate natural killer (NK) cell function to protect cells from NK-mediated cell lysis. KIRs are generally expressed on NK cells but have also been detected on tumor specific CTLs. Multiple immune checkpoint modulators specific for KIR have been developed and may be used as disclosed herein. In some embodiments, the immune checkpoint modulator is an agent that modulates the activity and/or expression of KIR. In some embodiments, the immune checkpoint modulator is an agent that binds to KIR (e.g., an anti-KIR antibody). In some embodiments, the immune checkpoint modulator is a KIR-binding protein (e.g., an antibody). In some embodiments, the checkpoint modulator is an KIR agonist. In some embodiments, the checkpoint modulator is an KIR antagonist. In some embodiments the immune checkpoint modulator is lirilumab (also known as BMS-986015; Bristol-Myers Squibb). Additional KIR binding proteins (e.g., antibodies) are known in the art and are disclosed, e.g., in U.S. Pat. Nos. 8,981,065, 9,018,366, 9,067,997, 8,709,411, 8,637,258, 8,614,307, 8,551,483, 8,388,970, 8,119,775; U.S. Patent Application Publication Nos. 2015/0344576, 2015/0376275, 2016/0046712, 2015/0191547, 2015/0290316, 2015/0283234, 2015/0197569, 2014/0193430, 2013/0143269, 2013/0287770, 2012/0208237, 2011/0293627, 2009/0081240, 2010/0189723; and PCT Publication Nos. WO 2016/069589, WO 2015/069785, WO 2014/066532, WO 2014/055648, WO 2012/160448, WO 2012/071411, WO 2010/065939, WO 2008/084106, WO 2006/072625, WO 2006/072626, and WO 2006/003179, each of which is incorporated by reference herein.

LAG-3, Lymphocyte-activation gene 3 (LAG-3, also known as CD223) is a CD4-related transmembrane protein that competitively binds MHC II and acts as a co-inhibitory checkpoint for T cell activation (see, e.g., Goldberg and Drake (2011) Curr. Top. Microbiol. Immunol. 344: 269-78). Multiple immune checkpoint modulators specific for LAG-3 have been developed and may be used as disclosed herein. In some embodiments, the immune checkpoint modulator is an agent that modulates the activity and/or expression of LAG-3. In some embodiments, the immune checkpoint modulator is an agent that binds to LAG-3 (e.g., an anti-PD-1 antibody). In some embodiments, the checkpoint modulator is an LAG-3 agonist. In some embodiments, the checkpoint modulator is an LAG-3 antagonist. In some embodiments, the immune checkpoint modulator is a LAG-3-binding protein (e.g., an antibody) selected from the group consisting of pembrolizumab (Keytruda; formerly lambrolizumab; Merck & Co., Inc.), nivolumab (Opdivo; Bristol-Myers Squibb), pidilizumab (CT-011, CureTech), SHR-1210 (Incyte/Jiangsu Hengrui Medicine Co., Ltd.), MEDIO680 (also known as AMP-514; Amplimmune Inc./Medimmune), PDR001 (Novartis), BGB-A317 (BeiGene Ltd.), TSR-042 (also known as ANBO11; AnaptysBio/Tesaro, Inc.), REGN2810 (Regeneron Pharmaceuticals, Inc./Sanofi-Aventis), and PF-06801591 (Pfizer). Additional PD-1-binding proteins (e.g., antibodies) are known in the art and are disclosed, e.g., in U.S. Pat. Nos. 9,181,342, 8,927,697, 7,488,802, 7,029,674; U.S. Patent Application Publication Nos. 2015/0152180, 2011/0171215, 2011/0171220; and PCT Publication Nos. WO 2004/056875, WO 2015/036394, WO 2010/029435, WO 2010/029434, WO 2014/194302, each of which is incorporated by reference herein.

PD-1. Programmed cell death protein 1 (PD-1, also known as CD279 and PDCD1) is an inhibitory receptor that negatively regulates the immune system. In contrast to CTLA-4 which mainly affects naïve T cells, PD-1 is more broadly expressed on immune cells and regulates mature T cell activity in peripheral tissues and in the tumor microenvironment. PD-1 inhibits T cell responses by interfering with T cell receptor signaling. PD-1 has two ligands, PD-L1 and PD-L2. Multiple immune checkpoint modulators specific for PD-1 have been developed and may be used as disclosed herein. In some embodiments, the immune checkpoint modulator is an agent that modulates the activity and/or expression of PD-1. In some embodiments, the immune checkpoint modulator is an agent that binds to PD-1 (e.g., an anti-PD-1 antibody). In some embodiments, the checkpoint modulator is an PD-1 agonist. In some embodiments, the checkpoint modulator is an PD-1 antagonist. In some embodiments, the immune checkpoint modulator is a PD-1-binding protein (e.g., an antibody) selected from the group consisting of pembrolizumab (Keytruda; formerly lambrolizumab; Merck & Co., Inc.), nivolumab (Opdivo; Bristol-Myers Squibb), pidilizumab (CT-011, CureTech), SHR-1210 (Incyte/Jiangsu Hengrui Medicine Co., Ltd.), MEDIO680 (also known as AMP-514; Amplimmune Inc./Medimmune), PDR001 (Novartis), BGB-A317 (BeiGene Ltd.), TSR-042 (also known as ANBO11; AnaptysBio/Tesaro, Inc.), REGN2810 (Regeneron Pharmaceuticals, Inc./Sanofi-Aventis), and PF-06801591 (Pfizer). Additional PD-1-binding proteins (e.g., antibodies) are known in the art and are disclosed, e.g., in U.S. Pat. Nos. 9,181,342, 8,927,697, 7,488,802, 7,029,674; U.S. Patent Application Publication Nos. 2015/0152180, 2011/0171215, 2011/0171220; and PCT Publication Nos. WO 2004/056875, WO 2015/036394, WO 2010/029435, WO 2010/029434, WO 2014/194302, each of which is incorporated by reference herein.

In some embodiments, one or more recombinant nucleic acid molecules encoding TRIF and Gasdermin E as described herein are administered in combination with a PD-1 antagonist, e.g., an anti-PD-1 binding protein (e.g., anti-PD-1 antibody) as described above. In some embodiments, administration of the one or more recombinant nucleic acid molecules encoding TRIF and Gasdermin E and the PD-1 antagonist (e.g., anti-PD-1 antibody) provide a synergistic therapeutic effect, e.g, immune-mediated inhibition of tumor growth or regression of tumors (e.g., metastatic tumors).

PD-L1/PD-L2. PD ligand 1 (PD-L1, also known as B7-H1) and PD ligand 2 (PD-L2, also known as PDCDILG2, CD273, and B7-DC) bind to the PD-1 receptor. Both ligands belong to the same B7 family as the B7-1 and B7-2 proteins that interact with CD28 and CTLA-4. PD-L1 can be expressed on many cell types including, for example, epithelial cells, endothelial cells, and immune cells. Ligation of PDL-1 decreases IFNγ, TNFα, and IL-2 production and stimulates production of IL10, an anti-inflammatory cytokine associated with decreased T cell reactivity and proliferation as well as antigen-specific T cell anergy. PDL-2 is predominantly expressed on antigen presenting cells (APCs). PDL2 ligation also results in T cell suppression, but where PDL-1-PD-1 interactions inhibits proliferation via cell cycle arrest in the G1/G2 phase, PDL2-PD-1 engagement has been shown to inhibit TCR-mediated signaling by blocking B7:CD28 signals at low antigen concentrations and reducing cytokine production at high antigen concentrations. Multiple immune checkpoint modulators specific for PD-L1 and PD-L2 have been developed and may be used as disclosed herein.

In some embodiments, the immune checkpoint modulator is an agent that modulates the activity and/or expression of PD-L1. In some embodiments, the immune checkpoint modulator is an agent that binds to PD-L1 (e.g., an anti-PD-L1 antibody). In some embodiments, the checkpoint modulator is an PD-L1 agonist. In some embodiments, the checkpoint modulator is an PD-L1 antagonist. In some embodiments, the immune checkpoint modulator is a PD-L1-binding protein (e.g., an antibody or a Fc-fusion protein) selected from the group consisting of durvalumab (also known as MEDI-4736; AstraZeneca/Celgene Corp./Medimmune), atezolizumab (Tecentriq; also known as MPDL3280A and RG7446; Genetech Inc.), avelumab (also known as MSB0010718C; Merck Serono/AstraZeneca); MDX-1105 (Medarex/Bristol-Meyers Squibb), AMP-224 (Amplimmune, GlaxoSmithKline), LY3300054 (Eli Lilly and Co.). Additional PD-L1-binding proteins are known in the art and are disclosed, e.g., in U.S. Patent Application Publication Nos. 2016/0084839, 2015/0355184, 2016/0175397, and PCT Publication Nos. WO 2014/100079, WO 2016/030350, WO2013181634, each of which is incorporated by reference herein.

In some embodiments, the immune checkpoint modulator is an agent that modulates the activity and/or expression of PD-L2. In some embodiments, the immune checkpoint modulator is an agent that binds to PD-L2 (e.g., an anti-PD-L2 antibody). In some embodiments, the checkpoint modulator is an PD-L2 agonist. In some embodiments, the checkpoint modulator is an PD-L2 antagonist. PD-L2-binding proteins (e.g., antibodies) are known in the art and are disclosed, e.g., in U.S. Pat. Nos. 9,255,147, 8,188,238; U.S. Patent Application Publication Nos. 2016/0122431, 2013/0243752, 2010/0278816, 2016/0137731, 2015/0197571, 2013/0291136, 2011/0271358; and PCT Publication Nos. WO 2014/022758, and WO 2010/036959, each of which is incorporated by reference herein.

TIM-3. T cell immunoglobulin mucin 3 (TIM-3, also known as Hepatitis A virus cellular receptor (HAVCR2)) is a type I glycoprotein receptor that binds to S-type lectin galectin-9 (Gal-9). TIM-3, is a widely expressed ligand on lymphocytes, liver, small intestine, thymus, kidney, spleen, lung, muscle, reticulocytes, and brain tissue. Tim-3 was originally identified as being selectively expressed on IFN-γ-secreting Th1 and Tcl cells (Monney et al. (2002) Nature 415: 536-41). Binding of Gal-9 by the TIM-3 receptor triggers downstream signaling to negatively regulate T cell survival and function. Multiple immune checkpoint modulators specific for TIM-3 have been developed and may be used as disclosed herein. In some embodiments, the immune checkpoint modulator is an agent that modulates the activity and/or expression of TIM-3. In some embodiments, the immune checkpoint modulator is an agent that binds to TIM-3 (e.g., an anti-TIM-3 antibody). In some embodiments, the checkpoint modulator is an TIM-3 agonist. In some embodiments, the checkpoint modulator is an TIM-3 antagonist. In some embodiments, the immune checkpoint modulator is an anti-TIM-3 antibody selected from the group consisting of TSR-022 (AnaptysBio/Tesaro, Inc.) and MGB453 (Novartis). Additional TIM-3 binding proteins (e.g., antibodies) are known in the art and are disclosed, e.g., in U.S. Pat. Nos. 9,103,832, 8,552,156, 8,647,623, 8,841,418; U.S. Patent Application Publication Nos. 2016/0200815, 2015/0284468, 2014/0134639, 2014/0044728, 2012/0189617, 2015/0086574, 2013/0022623; and PCT Publication Nos. WO 2016/068802, WO 2016/068803, WO 2016/071448, WO 2011/155607, and WO 2013/006490, each of which is incorporated by reference herein.

VISTA. V-domain Ig suppressor of T cell activation (VISTA, also known as Platelet receptor Gi24) is an Ig super-family ligand that negatively regulates T cell responses. See, e.g., Wang et al., 2011, J. Exp. Med. 208: 577-92. VISTA expressed on APCs directly suppresses CD4⁺ and CD8⁺ T cell proliferation and cytokine production (Wang et al. (2010) J Exp Med. 208(3): 577-92). Multiple immune checkpoint modulators specific for VISTA have been developed and may be used as disclosed herein. In some embodiments, the immune checkpoint modulator is an agent that modulates the activity and/or expression of VISTA. In some embodiments, the immune checkpoint modulator is an agent that binds to VISTA (e.g., an anti-VISTA antibody). In some embodiments, the checkpoint modulator is an VISTA agonist. In some embodiments, the checkpoint modulator is an VISTA antagonist. In some embodiments, the immune checkpoint modulator is a VISTA-binding protein (e.g., an antibody) selected from the group consisting of TSR-022 (AnaptysBio/Tesaro, Inc.) and MGB453 (Novartis). VISTA-binding proteins (e.g., antibodies) are known in the art and are disclosed, e.g., in U.S. Patent Application Publication Nos. 2016/0096891, 2016/0096891; and PCT Publication Nos. WO 2014/190356, WO 2014/197849, WO 2014/190356 and WO 2016/094837, each of which is incorporated by reference herein.

Methods are provided for the treatment of oncological disorders by administering a recombinant nucleic acid molecule, vector (e.g. an engineered virus, plasmid or transposon), cell or pharmaceutical composition as described herein in combination with at least one immune checkpoint modulator to a subject. In certain embodiments, the immune checkpoint modulator stimulates the immune response of the subject. For example, in some embodiments, the immune checkpoint modulator stimulates or increases the expression or activity of a stimulatory immune checkpoint (e.g. CD27, CD28, CD40, CD122, OX40, GITR, ICOS, or 4-1BB). In some embodiments, the immune checkpoint modulator inhibits or decreases the expression or activity of an inhibitory immune checkpoint (e.g. A2A4, B7-H3, B7-H4, BTLA, CTLA-4, IDO, KIR, LAG3, PD-1, PD-L1, PD-L2, TIM-3 or VISTA).

In certain embodiments the immune checkpoint modulator targets an immune checkpoint molecule selected from the group consisting of CD27, CD28, CD40, CD122, OX40, GITR, ICOS, 4-1BB, A2A4, B7-H3, B7-H4, BTLA, CTLA-4, IDO, KIR, LAG3, PD-1, PD-L1, PD-L2, TIM-3 and VISTA. In certain embodiments the immune checkpoint modulator targets an immune checkpoint molecule selected from the group consisting of CD27, CD28, CD40, CD122, OX40, GITR, ICOS, 4-1BB, A2A4, B7-H3, B7-H4, BTLA, IDO, KIR, LAG3, PD-1, PD-L1, PD-L2, TIM-3 and VISTA. In a particular embodiment, the immune checkpoint modulator targets an immune checkpoint molecule selected from the group consisting of CTLA-4, PD-L1 and PD-1. In a further particular embodiment the immune checkpoint modulator targets an immune checkpoint molecule selected from PD-L1 and PD-1.

In some embodiments, more than one (e.g. 2, 3, 4, 5 or more) immune checkpoint modulator is administered to the subject. Where more than one immune checkpoint modulator is administered, the modulators may each target a stimulatory immune checkpoint molecule, or each target an inhibitory immune checkpoint molecule. In other embodiments, the immune checkpoint modulators include at least one modulator targeting a stimulatory immune checkpoint and at least one immune checkpoint modulator targeting an inhibitory immune checkpoint molecule. In certain embodiments, the immune checkpoint modulator is a binding protein, for example, an antibody. The term “binding protein”, as used herein, refers to a protein or polypeptide that can specifically bind to a target molecule, e.g. an immune checkpoint molecule. In some embodiments the binding protein is an antibody or antigen binding portion thereof, and the target molecule is an immune checkpoint molecule. In some embodiments the binding protein is a protein or polypeptide that specifically binds to a target molecule (e.g., an immune checkpoint molecule). In some embodiments the binding protein is a ligand. In some embodiments, the binding protein is a fusion protein. In some embodiments, the binding protein is a receptor. Examples of binding proteins that may be used in the methods of the invention include, but are not limited to, a humanized antibody, an antibody Fab fragment, a divalent antibody, an antibody drug conjugate, a scFv, a fusion protein, a bivalent antibody, and a tetravalent antibody.

The term “antibody”, as used herein, refers to any immunoglobulin (Ig) molecule comprised of four polypeptide chains, two heavy (H) chains and two light (L) chains, or any functional fragment, mutant, variant, or derivation thereof. Such mutant, variant, or derivative antibody formats are known in the art. In a full-length antibody, each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. Immunoglobulin molecules can be of any type (e.g., IgG, IgE, IgM, IgD, IgA and IgY), class (e.g., IgG 1, IgG2, IgG 3, IgG4, IgA1 and IgA2) or subclass. In some embodiments, the antibody is a full-length antibody. In some embodiments, the antibody is a murine antibody. In some embodiments, the antibody is a human antibody. In some embodiments, the antibody is a humanized antibody. In other embodiments, the antibody is a chimeric antibody. Chimeric and humanized antibodies may be prepared by methods well known to those of skill in the art including CDR grafting approaches (see, e.g., U.S. Pat. Nos. 5,843,708; 6,180,370; 5,693,762; 5,585,089; and 5,530,101), chain shuffling strategies (see, e.g., U.S. Pat. No. 5,565,332; Rader et al. (1998) PROC. NAT'L. ACAD. SCI. USA 95: 8910-8915), molecular modeling strategies (U.S. Pat. No. 5,639,641), and the like.

The term “antigen-binding portion” of an antibody (or simply “antibody portion”), as used herein, refers to one or more fragments of an antibody that retain the ability to specifically bind to an antigen. It has been shown that the antigen-binding function of an antibody can be performed by fragments of a full-length antibody. Such antibody embodiments may also be bispecific, dual specific, or multi-specific formats; specifically binding to two or more different antigens. Examples of binding fragments encompassed within the term “antigen-binding portion” of an antibody include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al. (1989) NATURE 341: 544-546; and WO 90/05144 A1, the contents of which are herein incorporated by reference), which comprises a single variable domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see, e.g., Bird et al. (1988) SCIENCE 242:423-426; and Huston et al. (1988) PROC. NAT'L. ACAD. SCI. USA 85:5879-5883). Such single chain antibodies are also intended to be encompassed within the term “antigen-binding portion” of an antibody. Other forms of single chain antibodies, such as diabodies are also encompassed. Antigen binding portions can also be incorporated into single domain antibodies, maxibodies, minibodies, nanobodies, intrabodies, diabodies, triabodies, tetrabodies, v-NAR and bis-scFv (see, e.g., Hollinger and Hudson, Nature Biotechnology 23:1126-1136, 2005).

As used herein, the term “CDR” refers to the complementarity determining region within antibody variable sequences. There are three CDRs in each of the variable regions of the heavy chain and the light chain, which are designated CDR1, CDR2 and CDR3, for each of the variable regions. The term “CDR set” as used herein refers to a group of three CDRs that occur in a single variable region capable of binding the antigen. The exact boundaries of these CDRs have been defined differently according to different systems. The system described by Kabat (Kabat et al., SEQUENCES OF PROTEINS OF IMMUNOLOGICAL INTEREST (National Institutes of Health, Bethesda, Md. (1987) and (1991)) not only provides an unambiguous residue numbering system applicable to any variable region of an antibody, but also provides precise residue boundaries defining the three CDRs. These CDRs may be referred to as Kabat CDRs. Chothia and coworkers found that certain sub-portions within Kabat CDRs adopt nearly identical peptide backbone conformations, despite having great diversity at the level of amino acid sequence (Chothia et al. (1987) J. MOL. BIOL. 196: 901-917, and Chothia et al. (1989) NATURE 342: 877-883). These sub-portions were designated as L1, L2 and L3 or H1, H2 and H3 where the “L” and the “H” designates the light chain and the heavy chains regions, respectively. These regions may be referred to as Chothia CDRs, which have boundaries that overlap with Kabat CDRs. Other boundaries defining CDRs overlapping with the Kabat CDRs have been described by Padlan et al. (1995) FASEB J. 9: 133-139, and MacCallum et al. (1996) J. MOL. BIOL. 262(5): 732-45. Still other CDR boundary definitions may not strictly follow one of the above systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems, although preferred embodiments use Kabat or Chothia defined CDRs.

The term “humanized antibody”, as used herein refers to non-human (e.g., murine) antibodies that are chimeric immunoglobulins, immunoglobulin chains, or fragments thereof (such as Fv, Fab, Fab′, F(ab′)2 or other antigen-binding subsequences of antibodies) which contain minimal sequence derived from a non-human immunoglobulin. For the most part, humanized antibodies and antibody fragments thereof are human immunoglobulins (recipient antibody or antibody fragment) in which residues from a complementary-determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, a humanized antibody/antibody fragment can comprise residues which are found neither in the recipient antibody nor in the imported CDR or framework sequences. These modifications can further refine and optimize antibody or antibody fragment performance. In general, the humanized antibody or antibody fragment thereof will comprise substantially all of at least one, and typically two, variable domains, in which all or substantially all of the CDR regions correspond to those of a non-human immunoglobulin and all or a significant portion of the FR regions are those of a human immunoglobulin sequence. The humanized antibody or antibody fragment can also comprise at least a portion of an immunoglobulin constant region (Fc), typically that of a human immunoglobulin. For further details, see Jones et al. (1986) NATURE 321: 522-525; Reichmann et al. (1988) NATURE 332: 323-329; and Presta (1992) CURR. OP. STRUCT. BIOL. 2: 593-596, each of which is incorporated by reference herein in its entirety.

The term “immunoconjugate” or “antibody drug conjugate” as used herein refers to the linkage of an antibody or an antigen binding fragment thereof with another agent, such as a chemotherapeutic agent, a toxin, an immunotherapeutic agent, an imaging probe, and the like. The linkage can be covalent bonds, or non-covalent interactions such as through electrostatic forces. Various linkers, known in the art, can be employed in order to form the immunoconjugate. Additionally, the immunoconjugate can be provided in the form of a fusion protein that may be expressed from a polynucleotide encoding the immunoconjugate.

As used herein, “fusion protein” refers to proteins created through the joining of two or more genes or gene fragments which originally coded for separate proteins (including peptides and polypeptides). Translation of the fusion gene results in a single protein with functional properties derived from each of the original proteins.

A “bivalent antibody” refers to an antibody or antigen-binding fragment thereof that comprises two antigen-binding sites. The two antigen binding sites may bind to the same antigen, or they may each bind to a different antigen, in which case the antibody or antigen-binding fragment is characterized as “bispecific.” A “tetravalent antibody” refers to an antibody or antigen-binding fragment thereof that comprises four antigen-binding sites. In certain embodiments, the tetravalent antibody is bispecific. In certain embodiments, the tetravalent antibody is multispecific, i.e. binding to more than two different antigens.

Fab (fragment antigen binding) antibody fragments are immunoreactive polypeptides comprising monovalent antigen-binding domains of an antibody composed of a polypeptide consisting of a heavy chain variable region (VH) and heavy chain constant region 1 (C_{H1}) portion and a poly peptide consisting of a light chain variable (V_{L}) and light chain constant (C_{L}) portion, in which the C_{L} and C_{H1} portions are bound together, preferably by a disulfide bond between Cys residues.

Immune checkpoint modulator antibodies include, but are not limited to, at least 4 major categories: i) antibodies that block an inhibitory pathway directly on T cells or natural killer (NK) cells (e.g., PD-1 targeting antibodies such as nivolumab and pembrolizumab, antibodies targeting TIM-3, and antibodies targeting LAG-3, 2B4, CD160, A2aR, BTLA, CGEN-15049, and KIR), ii) antibodies that activate stimulatory pathways directly on T cells or NK cells (e.g., antibodies targeting OX40, GITR, and 4-1BB), iii) antibodies that block a suppressive pathway on immune cells or relies on antibody-dependent cellular cytotoxicity to deplete suppressive populations of immune cells (e.g., CTLA-4 targeting antibodies such as ipilimumab, antibodies targeting VISTA, and antibodies targeting PD-L2, Gr1, and Ly6G), and iv) antibodies that block a suppressive pathway directly on cancer cells or that rely on antibody-dependent cellular cytotoxicity to enhance cytotoxicity to cancer cells (e.g., rituximab, antibodies targeting PD-L1, and antibodies targeting B7-H3, B7-H4, Gal-9, and MUC1). Examples of checkpoint inhibitors include, e.g., an inhibitor of CTLA-4, such as ipilimumab or tremelimumab; an inhibitor of the PD-1 pathway such as an anti-PD-1, anti-PD-L1 or anti-PD-L2 antibody. Exemplary anti-PD-1 antibodies are described in WO 2006/121168, WO 2008/156712, WO 2012/145493, WO 2009/014708 and WO 2009/114335. Exemplary anti-PD-L1 antibodies are described in WO 2007/005874, WO 2010/077634 and WO 2011/066389, and exemplary anti-PD-L2 antibodies are described in WO 2004/007679.

In a particular embodiment, the immune checkpoint modulator is a fusion protein, for example, a fusion protein that modulates the activity of an immune checkpoint modulator.

In one embodiment, the immune checkpoint modulator is a therapeutic nucleic acid molecule, for example a nucleic acid that modulates the expression of an immune checkpoint protein or mRNA. Nucleic acid therapeutics are well known in the art. Nucleic acid therapeutics include both single stranded and double stranded (i.e., nucleic acid therapeutics having a complementary region of at least 15 nucleotides in length) nucleic acids that are complementary to a target sequence in a cell. In certain embodiments, the nucleic acid therapeutic is targeted against a nucleic acid sequence encoding an immune checkpoint protein.

Antisense nucleic acid therapeutic agents are single stranded nucleic acid therapeutics, typically about 16 to 30 nucleotides in length, and are complementary to a target nucleic acid sequence in the target cell, either in culture or in an organism.

In another aspect, the agent is a single-stranded antisense RNA molecule. An antisense RNA molecule is complementary to a sequence within the target mRNA. Antisense RNA can inhibit translation in a stoichiometric manner by base pairing to the mRNA and physically obstructing the translation machinery, see Dias, N. et al., (2002) Mol Cancer Ther 1:347-355. The antisense RNA molecule may have about 15-30 nucleotides that are complementary to the target mRNA. Patents directed to antisense nucleic acids, chemical modifications, and therapeutic uses include, for example: U.S. Pat. No. 5,898,031 related to chemically modified RNA-containing therapeutic compounds; U.S. Pat. No. 6,107,094 related methods of using these compounds as therapeutic agents; U.S. Pat. No. 7,432,250 related to methods of treating patients by administering single-stranded chemically modified RNA-like compounds; and U.S. Pat. No. 7,432,249 related to pharmaceutical compositions containing single-stranded chemically modified RNA-like compounds. U.S. Pat. No. 7,629,321 is related to methods of cleaving target mRNA using a single-stranded oligonucleotide having a plurality of RNA nucleosides and at least one chemical modification. The entire contents of each of the patents listed in this paragraph are incorporated herein by reference.

Nucleic acid therapeutic agents for use in the methods of the invention also include double stranded nucleic acid therapeutics. An “RNAi agent,” “double stranded RNAi agent,” double-stranded RNA (dsRNA) molecule, also referred to as “dsRNA agent,” “dsRNA”, “siRNA”, “iRNA agent,” as used interchangeably herein, refers to a complex of ribonucleic acid molecules, having a duplex structure comprising two anti-parallel and substantially complementary, as defined below, nucleic acid strands. As used herein, an RNAi agent can also include dsiRNA (see, e.g., US Patent publication 20070104688, incorporated herein by reference). In general, the majority of nucleotides of each strand are ribonucleotides, but as described herein, each or both strands can also include one or more non-ribonucleotides, e.g., a deoxyribonucleotide and/or a modified nucleotide. In addition, as used in this specification, an “RNAi agent” may include ribonucleotides with chemical modifications; an RNAi agent may include substantial modifications at multiple nucleotides. Such modifications may include all types of modifications disclosed herein or known in the art. Any such modifications, as used in a siRNA type molecule, are encompassed by “RNAi agent” for the purposes of this specification and claims. The RNAi agents that are used in the methods of the invention include agents with chemical modifications as disclosed, for example, in WO/2012/037254, and WO 2009/073809, the entire contents of each of which are incorporated herein by reference.

Immune checkpoint modulators may be administered at appropriate dosages to treat the oncological disorder, for example, by using standard dosages. One skilled in the art would be able, by routine experimentation, to determine what an effective, non-toxic amount of an immune checkpoint modulator would be for the purpose of treating oncological disorders. Standard dosages of immune checkpoint modulators are known to a person skilled in the art and may be obtained, for example, from the product insert provided by the manufacturer of the immune checkpoint modulator. Examples of standard dosages of immune checkpoint modulators are provided in Table 3 below. In other embodiments, the immune checkpoint modulator is administered at a dosage that is different (e.g., lower) than the standard dosages of the immune checkpoint modulator used to treat the oncological disorder under the standard of care for treatment for a particular oncological disorder.

TABLE 3
Exemplary Standard Dosages of Immune Checkpoint Modulators

| | Immune | |
|---|---|---|
| Immune | Checkpoint | |
| Checkpoint | Molecule | |
| Modulator | Targeted | Exemplary Standard Dosage |
| Ipilimumab | CTLA-4 | 3 mg/kg administered intravenously over |
| (Yervoy ™) | | 90 minutes every 3 weeks for a total of |
| | | 4 doses |
| Pembrolizumab | PD-1 | 2 mg/kg administered as an intravenous |
| (Keytruda ™) | | infusion over 30 minutes every 3 weeks |
| | | until disease progression or unacceptable |
| | | toxicity |
| Atezolizumab | PD-L1 | 1200 mg administered as an intravenous |
| (Tecentriq ™) | | infusion over 60 minutes every 3 weeks |

In certain embodiments, the administered dosage of the immune checkpoint modulator is 5%, 10%, 20%, 30%, 40%, 50%, 60%, 70%, 80%, or 90% lower than the standard dosage of the immune checkpoint modulator for a particular oncological disorder. In certain embodiments, the dosage administered of the immune checkpoint modulator is 95%, 90%, 85%, 80%, 75%, 70%, 65%, 60%, 55%, 50%, 45%, 40%, 35%, 30%, 25%, 20%, 15%, 10% or 5% of the standard dosage of the immune checkpoint modulator for a particular oncological disorder. In one embodiment, where a combination of immune checkpoint modulators are administered, at least one of the immune checkpoint modulators is administered at a dose that is lower than the standard dosage of the immune checkpoint modulator for a particular oncological disorder. In one embodiment, where a combination of immune checkpoint modulators are administered, at least two of the immune checkpoint modulators are administered at a dose that is lower than the standard dosage of the immune checkpoint modulators for a particular oncological disorder. In one embodiment, where a combination of immune checkpoint modulators are administered, at least three of the immune checkpoint modulators are administered at a dose that is lower than the standard dosage of the immune checkpoint modulators for a particular oncological disorder. In one embodiment, where a combination of immune checkpoint modulators are administered, all of the immune checkpoint modulators are administered at a dose that is lower than the standard dosage of the immune checkpoint modulators for a particular oncological disorder.

Additional immunotherapeutics that may be administered in combination with the recombinant nucleic acid molecule, vector (e.g. an engineered virus, plasmid or transposon), cell or pharmaceutical composition as described herein include, but are not limited to, Toll-like receptor (TLR) agonists, cell-based therapies, cytokines and cancer vaccines.

### 2. TLR Agonists

TLRs are single membrane-spanning non-catalytic receptors that recognize structurally conserved molecules derived from microbes. TLRs together with the Interleukin-1 receptor form a receptor superfamily, known as the “Interleukin-1 Receptor/Toll-Like Receptor Superfamily.” Members of this family are characterized structurally by an extracellular leucine-rich repeat (LRR) domain, a conserved pattern of juxtamembrane cysteine residues, and an intracytoplasmic signaling domain that forms a platform for downstream signaling by recruiting TIR domain-containing adapters including MyD88, TIR domain-containing adaptor (TRAP), and TIR domain-containing adaptor inducing IFNβ (TRIF) (O'Neill et al., 2007, Nat Rev Immunol 7, 353).

The TLRs include TLR1, TLR2, TLR3, TLR4, TLR5, TLR6, TLR7, TLR8, TLR9, and TLR10. TLR2 mediates cellular responses to a large number of microbial products including peptidoglycan, bacterial lipopeptides, lipoteichoic acid, mycobacterial lipoarabinomannan and yeast cell wall components. TLR4 is a transmembrane protein which belongs to the pattern recognition receptor (PRR) family. Its activation leads to an intracellular signaling pathway NF-κB and inflammatory cytokine production which is responsible for activating the innate immune system. TLR5 is known to recognize bacterial flagellin from invading mobile bacteria, and has been shown to be involved in the onset of many diseases, including inflammatory bowel disease.

TLR agonists are known in the art and are described, for example, in US2014/0030294, which is incorporated by reference herein in its entirety. Exemplary TLR2 agonists include mycobacterial cell wall glycolipids, lipoarabinomannan (LAM) and mannosylated phosphatidylinositol (PIIM), MALP-2 and Pam3Cys and synthetic variants thereof. Exemplary TLR4 agonists include lipopolysaccharide or synthetic variants thereof (e.g., MPL and RC529) and lipid A or synthetic variants thereof (e.g., aminoalkyl glucosaminide 4-phosphates). See, e.g., Cluff et al., 2005, Infection and Immunity, p. 3044-3052:73; Lembo et al., 2008, The Journal of Immunology 180, 7574-7581; and Evans et al., 2003, Expert Rev Vaccines 2:219-29. Exemplary TLR5 agonists include flagellin or synthetic variants thereof (e.g., A pharmacologically optimized TLR5 agonist with reduced immunogenicity (such as CBLB502) made by deleting portions of flagellin that are non-essential for TLR5 activation).

Additional TLR agonists include Coley's toxin and Bacille Calmette-Guerin (BCG). Coley's toxin is a mixture consisting of killed bacteria of species Streptococcus pyogenes and Serratia marcescens. See Taniguchi et al., 2006, Anticancer Res. 26 (6A): 3997-4002. BCG is prepared from a strain of the attenuated live bovine tuberculosis bacillus, Mycobacterium bovis. See Venkataswamy et al., 2012, Vaccine. 30 (6): 1038-1049.

### 3. Cell Based Therapies

Cell-based therapies for the treatment of cancer include administration of immune cells (e.g. T cells, tumor-infiltrating lymphocytes (TILs), Natural Killer cells, macrophages and dendritic cells) to a subject. In autologous cell-based therapy, the immune cells are derived from the same subject to which they are administered. In allogeneic cell-based therapy, the immune cells are derived from one subject and administered to a different subject. The immune cells may be activated, for example, by treatment with a cytokine, before administration to the subject. In some embodiments, the immune cells are genetically modified before administration to the subject, for example, as in chimeric antigen receptor (CAR) T cell immunotherapy.

In some embodiments, the cell-based therapy includes an adoptive cell transfer (ACT). ACT typically consists of three parts: lympho-depletion, cell administration, and therapy with high doses of IL-2. Types of cells that may be administered in ACT include tumor infiltrating lymphocytes (TILs), T cell receptor (TCR)-transduced T cells, and chimeric antigen receptor (CAR) T cells.

Tumor-infiltrating lymphocytes are immune cells that have been observed in many solid tumors, including breast cancer. They are a population of cells comprising a mixture of cytotoxic T cells and helper T cells, as well as B cells, macrophages, natural killer cells, and dendritic cells. The general procedure for autologous TIL therapy is as follows: (1) a resected tumor is digested into fragments; (2) each fragment is grown in IL-2 and the lymphocytes proliferate destroying the tumor; (3) after a pure population of lymphocytes exists, these lymphocytes are expanded; and (4) after expansion up to 10¹¹ cells, lymphocytes are infused into the patient. See Rosenberg et al., 2015, Science 348(6230):62-68, which is incorporated by reference herein in its entirety.

TCR-transduced T cells are generated via genetic induction of tumor-specific TCRs. This is often done by cloning the particular antigen-specific TCR into a retroviral backbone. Blood is drawn from patients and peripheral blood mononuclear cells (PBMCs) are extracted. PBMCs are stimulated with CD3 in the presence of IL-2 and then transduced with the retrovirus encoding the antigen-specific TCR. These transduced PBMCs are expanded further in vitro and infused back into patients. See Robbins et al., 2015, Clinical Cancer Research 21(5):1019-1027, which is incorporated by reference herein in its entirety.

Chimeric antigen receptors (CARs) are recombinant receptors containing an extracellular antigen recognition domain, a transmembrane domain, and a cytoplasmic signaling domain (such as CD3ζ, CD28, and 4-1BB). CARs possess both antigen-binding and T-cell-activating functions. Therefore, T cells expressing CARs can recognize a wide range of cell surface antigens, including glycolipids, carbohydrates, and proteins, and can attack malignant cells expressing these antigens through the activation of cytoplasmic costimulation. See Pang et al., 2018, Mol Cancer 17: 91, which is incorporated by reference herein in its entirety.

In some embodiments, the cell-based therapy is a Natural Killer (NK) cell-based therapy. NK cells are large, granular lymphocytes that have the ability to kill tumor cells without any prior sensitization or restriction of major histocompatibility complex (MHC) molecule expression. See Uppendahl et al., 2017, Frontiers in Immunology 8: 1825. Adoptive transfer of autologous lymphokine-activated killer (LAK) cells with high-dose IL-2 therapy have been evaluated in human clinical trials. Similar to LAK immunotherapy, cytokine-induced killer (CIK) cells arise from peripheral blood mononuclear cell cultures with stimulation of anti-CD3 mAb, IFN-γ, and IL-2. CIK cells are characterized by a mixed T-NK phenotype (CD3+CD56+) and demonstrate enhanced cytotoxic activity compared to LAK cells against ovarian and cervical cancer. Human clinical trials investigating adoptive transfer of autologous CIK cells following primary debulking surgery and adjuvant carboplatin/paclitaxel chemotherapy have also been conducted. See Liu et al., 2014, J Immunother 37(2): 116-122.

In some embodiments, the cell-based therapy is a dendritic cell-based immunotherapy. Vaccination with dendritic cells (DC)s treated with tumor lysates has been shown to increase therapeutic antitumor immune responses both in vitro and in vivo. See Jung et al., 2018, Translational Oncology 11(3): 686-690. DCs capture and process antigens, migrate into lymphoid organs, express lymphocyte costimulatory molecules, and secrete cytokines that initiate immune responses. They also stimulate immunological effector cells (T cells) that express receptors specific for tumor-associated antigens and reduce the number of immune repressors such as CD4+CD25+Foxp3+ regulatory T (Treg) cells. For example, a DC vaccination strategy for renal cell carcinoma (RCC), which is based on a tumor cell lysate-DC hybrid, showed therapeutic potential in preclinical and clinical trials. See Lim et al., 2007, Cancer Immunol Immunother 56: 1817-1829.

In some embodiments, the cell-based therapy includes mesenchymal stem cells (MSCs). Mesenchymal stem cells (MSCs) are adult stem cells capable of self-renewal and multilineage differentiation. The therapeutic benefits of MSCs have prompted their use in cell-based strategies to treat different diseases, including cancer. Tumors exert chemoattractant effects on MSCs that influence their recruitment to tumor sites. Once the tumor environment is reached, MSCs interact with cancer cells via direct and indirect mechanisms that affect tumor development. The paracrine function of MSCs is one of the main mechanisms involved in cancer regulation and is mediated by multiple factors, including growth factors and cytokines. These paracrine factors affect cellular processes involving the tumor cell cycle (i.e., cell proliferation), cell survival, angiogenesis, and immunosuppression/immunomodulation, allowing MSCs to regulate cancer. The interaction of MSCs with the tumor cell cycle is one of the ways by which MSCs exert their therapeutic effects. By inhibiting proliferation-related signaling pathways, such as the phosphatidylinositol 3-kinase/protein kinase B (PI3K/AKT), MSCs can induce cell cycle arrest and reduce cancer growth. In addition, MSCs can undergo differentiation into other cell types, such as cancer-associated fibroblasts (CAFs), to directly contribute to cancer progression. See Hmadcha et al., 2020, Front. Bioeng. Biotechnol 8(43): 1-13; doi.org/10.3389/fbioe.2020.00043.

In some embodiments, the cell-based therapy includes fibroblasts, e.g., cancer-associated fibroblasts (CAFs). These cells modulate cancer metastasis through synthesis and remodelling of the extracellular matrix (ECM) and production of growth factors, and influence angiogenesis, tumor mechanics, drug access and therapy responses. CAFs may also modulate the immune system. Targeting CAFs, by altering their numbers, subtype or functionality, provides a method of improving cancer therapies. See Sahai et al., 2020, Nature Reviews Cancer 20: 174-186.

### 4. Cytokines

Several cytokines including IL-2, IL-12, IL-15, IL-18, and IL-21 have been used in the treatment of cancer for activation of immune cells such as NK cells and T cells. IL-2 was one of the first cytokines used clinically, with hopes of inducing antitumor immunity. As a single agent at high dose IL-2 induces remissions in some patients with renal cell carcinoma (RCC) and metastatic melanoma. Low dose IL-2 has also been investigated and aimed at selectively ligating the IL-2 αβγ receptor (IL-2Rαβγ) in an effort to reduce toxicity while maintaining biological activity. See Romee et al., 2014, Scientifica, Volume 2014, Article ID 205796, 18 pages, which is incorporated by reference herein in its entirety.

Interleukin-15 (IL-15) is a cytokine with structural similarity to Interleukin-2 (IL-2). Like IL-2, IL-15 binds to and signals through a complex composed of IL-2/IL-15 receptor beta chain (CD122) and the common gamma chain (gamma-C, CD132). Recombinant IL-15 has been evaluated for treatment of solid tumors (e.g. melanoma, renal cell carcinoma) and to support NK cells after adoptive transfer in cancer patients. See Romee et al., cited above.

IL-12 is a heterodimeric cytokine composed of p35 and p40 subunits (IL-12α and β chains, respectively), originally identified as “NK cell stimulatory factor (NKSF)” based on its ability to enhance NK cell cytotoxicity. Upon encounter with pathogens, IL-12 is released by activated dendritic cells and macrophages and binds to its cognate receptor, which is primarily expressed on activated T and NK cells. Numerous preclinical studies have suggested that IL-12 has antitumor potential. See Romee et al., cited above. Interleukin-12 (IL-12) also plays an important role in innate and adaptive immunity. Gately, M K et al., Annu Rev Immunol. 16: 495-521 (1998). IL-12 functions primarily as a 70 kDa heterodimeric protein consisting of two disulfide-linked p35 and p40 subunits. The precursor form of the IL12 p40 subunit (NM_002187; P29460; also referred to as IL12B) is 328 amino acids in length, while its mature form is 306 amino acids long. The precursor form of the IL12 p35 subunit (NM_000882; P29459; also referred to as IL12A) is 219 amino acids in length and the mature form is 197 amino acids long. Id. The genes for the IL12 p35 and p40 subunits reside on different chromosomes and are regulated independently of each other. Gately, M K et al., Annu Rev Immunol. 16: 495-521 (1998). The active heterodimer (referred to as ‘p70’), is formed following protein synthesis.

In some embodiments, the IL-12 polypeptide of the present disclosure comprises a single polypeptide chain comprising the p35 and p40 subunits fused directly or by a linker. In other embodiments, the IL-12 polypeptide of the present disclosure comprises two polypeptide chains, the first polypeptide chain comprising the p35 subunit and the second polypeptide comprising the p40 subunit.

IL-18 is a member of the proinflammatory IL-1 family and, like IL-12, is secreted by activated phagocytes. IL-18 has demonstrated significant antitumor activity in preclinical animal models, and has been evaluated in human clinical trials. See Robertson et al., 2006, Clinical Cancer Research 12: 4265-4273.

IL-21 has been used for antitumor immunotherapy due to its ability to stimulate NK cells and CD8+ T cells. For ex vivo NK cell expansion, membrane bound IL-21 has been expressed in K562 stimulator cells, with effective results. See Denman et al., 2012, PLoS One 7(1)e30264. Recombinant human IL-21 was also shown to increase soluble CD25 and induce expression of perforin and granzyme B on CD8+ cells. IL-21 has been evaluated in several clinical trials for treatment of solid tumors. See Romee et al., cited above.

### 5. Cancer Vaccines

Therapeutic cancer vaccines eliminate cancer cells by strengthening a patients' own immune responses to the cancer, particularly CD8+ T cell mediated responses, with the assistance of suitable adjuvants. The therapeutic efficacy of cancer vaccines is dependent on the differential expression of tumor associated antigens (TAAs) by tumor cells relative to normal cells. TAAs derive from cellular proteins and should be mainly or selectively expressed on cancer cells to avoid either immune tolerance or autoimmunity effects. See Circelli et al., 2015, Vaccines 3(3): 544-555. Cancer vaccines include, for example, dendritic cell (DC) based vaccines, peptide/protein vaccines, genetic vaccines, and tumor cell vaccines. See Ye et al., 2018, J Cancer 9(2): 263-268.

The combination therapies of the present invention may be utilized for the treatment of oncological disorders. In some embodiments, the combination therapy of the recombinant nucleic acid molecule, vector (e.g. an engineered virus, plasmid or transposon), cell or pharmaceutical composition as described herein and the additional therapeutic agent inhibits tumor cell growth. Accordingly, the invention further provides methods of inhibiting tumor cell growth in a subject, comprising administering a recombinant nucleic acid molecule, vector (e.g. an engineered virus, plasmid or transposon), cell or pharmaceutical composition as described herein and at least one additional therapeutic agent to the subject, such that tumor cell growth is inhibited. In certain embodiments, treating cancer comprises extending survival or extending time to tumor progression as compared to a control. In some embodiments, the control is a subject that is treated with the additional therapeutic agent, but is not treated with the recombinant nucleic acid molecule, vector (e.g. an engineered virus, plasmid or transposon), cell or pharmaceutical composition as described herein. In some embodiments, the control is a subject that is treated with the recombinant nucleic acid molecule, vector (e.g. an engineered virus, plasmid or transposon), cell or pharmaceutical composition as described herein, but is not treated with the additional therapeutic agent. In some embodiments, the control is a subject that is not treated with the additional therapeutic agent or the recombinant nucleic acid molecule, vector (e.g. an engineered virus, plasmid or transposon), cell or pharmaceutical composition as described herein. In certain embodiments, the subject is a human subject. In some embodiments, the subject is identified as having a tumor prior to administration of the first dose of the nucleic acid molecule, vector (e.g. an engineered virus, plasmid or transposon), cell or pharmaceutical composition as described herein or the first dose of the additional therapeutic agent. In certain embodiments, the subject has a tumor at the time of the first administration of the recombinant nucleic acid molecule, vector (e.g. an engineered virus, plasmid or transposon), cell or pharmaceutical composition as described herein, or at the time of first administration of the additional therapeutic agent.

In certain embodiments, at least 1, 2, 3, 4, or 5 cycles of the combination therapy comprising the recombinant nucleic acid molecule, vector (e.g. an engineered virus, plasmid or transposon), cell or pharmaceutical composition as described herein and one or more additional therapeutic agents are administered to the subject. The subject is assessed for response criteria at the end of each cycle. The subject is also monitored throughout each cycle for adverse events (e.g., clotting, anemia, liver and kidney function, etc.) to ensure that the treatment regimen is being sufficiently tolerated.

It should be noted that more than one additional therapeutic agent, e.g., 2, 3, 4, 5, or more additional therapeutic agents, may be administered in combination with the recombinant nucleic acid molecule, vector (e.g. an engineered virus, plasmid or transposon), cell or pharmaceutical composition as described herein.

In one embodiment, administration of the recombinant nucleic acid molecule, vector (e.g. an engineered virus, plasmid or transposon), cell or pharmaceutical composition as described herein and the additional therapeutic agent as described herein results in one or more of, reducing tumor size, weight or volume, increasing time to progression, inhibiting tumor growth and/or prolonging the survival time of a subject having an oncological disorder. In certain embodiments, administration of the recombinant nucleic acid molecule, vector (e.g., an engineered virus, plasmid or transposon), cell or pharmaceutical composition as described herein and the additional therapeutic agent reduces tumor size, weight or volume, increases time to progression, inhibits tumor growth and/or prolongs the survival time of the subject by at least 1%, 2%, 3%, 4%, 5%, 10%, 20%, 30%, 40%, 50%, 60%, 70%, 80%, 90%, 100%, 200%, 300%, 400% or 500% relative to a corresponding control subject that is administered the recombinant nucleic acid molecule, vector (e.g. an engineered virus, plasmid or transposon), cell or pharmaceutical composition as described herein, but is not administered the additional therapeutic agent. In certain embodiments, administration of the recombinant nucleic acid molecule, vector (e.g., an engineered virus, plasmid or transposon), cell or pharmaceutical composition as described herein and the additional therapeutic agent reduces tumor size, weight or volume, increases time to progression, inhibits tumor growth and/or prolongs the survival time of a population of subjects afflicted with an oncological disorder by at least 1%, 2%, 3%, 4%, 5%, 10%, 20%, 30%, 40%, 50%, 60%, 70%, 80%, 90%, 100%, 200%, 300%, 400% or 500% relative to a corresponding population of control subjects afflicted with the oncological disorder that is administered the recombinant nucleic acid molecule, vector (e.g., an engineered virus, plasmid or transposon), cell or pharmaceutical composition as described herein, but is not administered the additional therapeutic agent. In other embodiments, administration of the recombinant nucleic acid molecule, vector (e.g., an engineered virus, plasmid or transposon), cell or pharmaceutical composition as described herein and the additional therapeutic agent stabilizes the oncological disorder in a subject with a progressive oncological disorder prior to treatment.

In certain embodiments, treatment with the recombinant nucleic acid molecule, vector (e.g. an engineered virus, plasmid or transposon), cell or pharmaceutical composition as described herein and the additional therapeutic agent (e.g. an immunotherapeutic) is combined with a further anti-neoplastic agent such as the standard of care for treatment of the particular cancer to be treated, for example by administering a standard dosage of one or more antineoplastic (e.g. chemotherapeutic) agents. The standard of care for a particular cancer type can be determined by one of skill in the art based on, for example, the type and severity of the cancer, the age, weight, gender, and/or medical history of the subject, and the success or failure of prior treatments. In certain embodiments of the invention, the standard of care includes any one of or a combination of surgery, radiation, hormone therapy, antibody therapy, therapy with growth factors, cytokines, and chemotherapy. In one embodiment, the additional anti-neoplastic agent is not an agent that induces iron-dependent cellular disassembly and/or an immune checkpoint modulator.

Additional anti-neoplastic agents suitable for use in the methods disclosed herein include, but are not limited to, chemotherapeutic agents (e.g., alkylating agents, such as Altretamine, Busulfan, Carboplatin, Carmustine, Chlorambucil, Cisplatin, Cyclophosphamide, Dacarbazine, Lomustine, Melphalan, Oxaliplatin, Temozolomide, Thiotepa; antimetabolites, such as 5-fluorouracil (5-FU), 6-mercaptopurine (6-MP); Capecitabine (Xeloda®), Cytarabine (Ara-C®), Floxuridine, Fludarabine, Gemcitabine (Gemzar®), Hydroxyurea, Methotrexate, Pemetrexed (Alimta®); anti-tumor antibiotics such as anthracyclines (e.g., Daunorubicin, Doxorubicin (Adriamycin®), Epirubicin, Idarubicin), Actinomycin-D, Bleomycin, Mitomycin-C, Mitoxantrone (also acts as a topoisomerase II inhibitor); topoisomerase inhibitors, such as Topotecan, Irinotecan (CPT-11), Etoposide (VP-16), Teniposide, Mitoxantrone (also acts as an anti-tumor antibiotic); mitotic inhibitors such as Docetaxel, Estramustine, Ixabepilone, Paclitaxel, Vinblastine, Vincristine, Vinorelbine; corticosteroids such as Prednisone, Methylprednisolone (Solumedrol®), Dexamethasone (Decadron®); enzymes such as L-asparaginase, and bortezomib (Velcade®)). Anti-neoplastic agents also include biologic anti-cancer agents, e.g., anti-TNF antibodies, e.g., adalimumab or infliximab; anti-CD20 antibodies, such as rituximab, anti-VEGF antibodies, such as bevacizumab; anti-HER2 antibodies, such as trastuzumab; anti-RSV, such as palivizumab.

### VII. Pharmaceutical Compositions and Modes of Administration

In certain aspects, the present disclosure relates to a pharmaceutical composition comprising a recombinant nucleic acid molecule, vector (e.g., an engineered virus, plasmid or transposon), cell or pharmaceutical composition as described herein. The pharmaceutical compositions described herein may be administered to a subject in any suitable formulation. These include, for example, liquid, semi-solid, and solid dosage forms. The preferred form depends on the intended mode of administration and therapeutic application.

In certain embodiments the pharmaceutical composition is suitable for oral administration. In certain embodiments, the pharmaceutical composition is suitable for parenteral administration, including topical administration and intravenous, intraperitoneal, intramuscular, and subcutaneous, injections. In a particular embodiment, the pharmaceutical composition is suitable for intravenous administration. In a further particular embodiment, the pharmaceutical composition is suitable for intratumoral administration.

Pharmaceutical compositions for parenteral administration include aqueous solutions of the active compounds in water-soluble form. For intravenous administration, the formulation may be an aqueous solution. The aqueous solution may include Hank's solution, Ringer's solution, phosphate buffered saline (PBS), physiological saline buffer or other suitable salts or combinations to achieve the appropriate pH and osmolarity for parenterally delivered formulations. Aqueous solutions can be used to dilute the formulations for administration to the desired concentration. The aqueous solution may contain substances which increase the viscosity of the solution, such as sodium carboxymethyl cellulose, sorbitol, or dextran. In some embodiments, the formulation includes a phosphate buffer saline solution which contains sodium phosphate dibasic, potassium phosphate monobasic, potassium chloride, sodium chloride and water for injection.

Formulations suitable for topical administration include liquid or semi-liquid preparations suitable for penetration through the skin, such as liniments, lotions, creams, ointments or pastes, and drops suitable for administration to the eye, ear, or nose. Formulations suitable for oral administration include preparations containing an inert diluent or an assimilable edible carrier. The formulation for oral administration may be enclosed in hard or soft shell gelatin capsule, or it may be compressed into tablets, or it may be incorporated directly with the food of the diet. When the dosage unit form is a capsule, it may contain, in addition to materials of the above type, a liquid carrier. Various other materials may be present as coatings or to otherwise modify the physical form of the dosage unit. Pharmaceutical compositions suitable for use in the present invention include compositions wherein the active ingredients (i.e., one or more recombinant nucleic acid molecules as described herein) are contained in an effective amount to achieve its intended purpose, e.g, promoting thanotransmission, increasing immune response, or treating cancer. In some embodiments, the pharmaceutical composition comprises a therapeutically effective amount of one or more recombinant nucleic acid molecules described herein. Determination of the effective amounts is well within the capability of those skilled in the art, especially in light of the detailed disclosure provided herein. In addition to the active ingredients, these pharmaceutical compositions may contain suitable pharmaceutically acceptable carriers including excipients and auxiliaries which facilitate processing of the active compounds into preparations which can be used pharmaceutically.

As will be readily apparent to one skilled in the art, the useful in vivo dosage to be administered and the particular mode of administration will vary depending upon the age, body weight, the severity of the affliction, and mammalian species treated, the particular compounds employed, and the specific use for which these compounds are employed. The determination of effective dosage levels, that is the dosage levels necessary to achieve the desired result, can be accomplished by one skilled in the art using routine methods, for example, human clinical trials, animal models, and in vitro studies.

In certain embodiments, the pharmaceutical composition is delivered orally. In certain embodiments, the composition is administered parenterally. In certain embodiments, the composition is delivered by injection or infusion. In certain embodiments, the composition is delivered topically including transmucosally. In certain embodiments, the composition is delivered by inhalation. In one embodiment, the compositions provided herein may be administered by injecting directly to a tumor. In some embodiments, the compositions may be administered by intravenous injection or intravenous infusion. In certain embodiments, administration is systemic. In certain embodiments, administration is local.

### EXAMPLES

This invention is further illustrated by the following examples which should not be construed as limiting. The contents of all references, Uniprot, Gene ID, Entrez Gene ID, GenBank Accession and Gene numbers, and published patents and patent applications cited throughout the application are hereby incorporated by reference in their entirety. Those skilled in the art will recognize that the invention may be practiced with variations on the disclosed structures, materials, compositions and methods, and such variations are regarded as within the ambit of the invention.

### Example 1. Induction of Cell Death in CT-26 Mouse Colon Carcinoma Cells Expressing One or More Thanotransmission Polypeptides

CT-26 mouse colon carcinoma cells (ATCC; CRL-2638) were transduced with lentivirus derived from the pLVX-Tet3G Vector (Takara; 631358) to establish stable Tet-On transactivator expression by the human PGK promotor. In the Tet-On system, gene expression is inducible by doxycycline. All lentiviral transductions were performed using standard production protocols utilizing 293T cells (ATCC; CRL-3216) and the Lentivirus Packaging Mix (Biosettia; pLV-PACK). CT-26-Tet3G cells were then transduced with the lentivirus expressing the human TRIF ORF (Accession No.: NM_182919) in pLVX-TRE3G (Takara; 631193). The CT-26-Tet3G cells were transduced alternatively, or in addition, with a vector expressing the mouse RIPK3 ORF (Accession No.: NM_019955.2); RIPK3 expression was driven by the constitutive PGK promotor derivative of pLV-EF1a-MCS-IRES-Hyg (Biosettia; cDNA-pLV02). Both ORFs were modified by the addition of two tandem DmrB domains that oligomerize upon binding to the B-B ligand (Takara; 635059), to allow for protein activation using the B/B homodimerizer (1 μM) to promote oligomerization. After initial testing, dimerization using the B/B did not have a substantial effect on the activity of the TRIF construct, but did promote activity of the RIPK3 expressing construct. Therefore, in all subsequent experiments, B/B-induced dimerization was not employed to activate any constructs including TRIF, but was only employed to activate single constructs expressing RIPK3. As such, B/B dimerizer was included in the experimental setup, to ensure that experimental conditions were comparable across all groups, although it had no effect on TRIF-induced activity. For example, as shown in FIG. 2B and described in Example 2, addition of the dimerizer had little effect on IRF activity in macrophages treated with cell culture from the engineered CT-26 cells described above.

CT26 mouse colon carcinoma cells expressing the indicated thanotransmission modules were seeded and subsequently treated for 24 h with doxycycline (1 mg/mL; Sigma Aldrich, 0219895525) and B/B homodimerizer (1 μM) to promote expression and protein activation via oligomerization. Relative cell viability was determined at 24 h post-treatment using the RealTime-Glo MT Cell Viability Assay kit (Promega, Catalogue No. G9712) as per the manufacturer's instructions and graphed showing the relative viability measured by relative luminescence units (RLU).

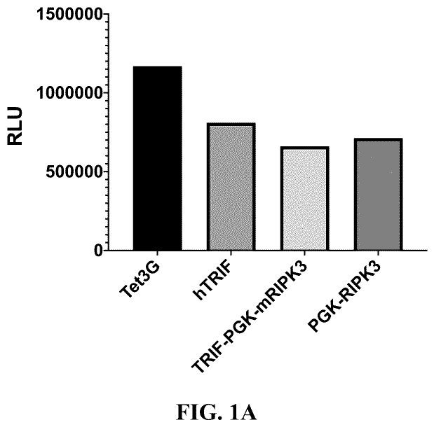

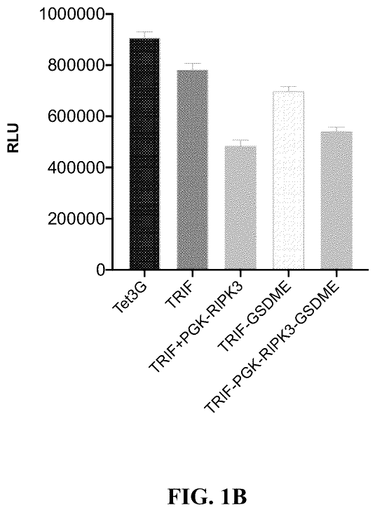

As shown in FIG. 1A, induced expression and oligomerization of TRIF, RIPK3, or TRIF+RIPK3 induced a reduction in cell viability relative to the CT-26-Tet3G (Tet3G) parental cell line. These results demonstrate that expression of one or more thanotransmission polypeptides in a cancer cell reduces viability of the cancer cell.

In a separate experiment, the effect of expression of Gasdermin E (GSDME) in cancer cells expressing TRIF, RIPK3, or TRIF and RIPK3 was examined. CT-26-Tet3G cells were transduced with human GSDME (NM_004403.3) cloned into the pLV-EF1a-MCS-IRES-Puro vector (Biosettia). GSDME was also transduced into the CT-26-Tet3G-TRIF and CT26-Tet3G-TRIF-RIPK3 cells described above. These cells were seeded and subsequently treated for 24 h with doxycycline (lmg/mL; Sigma Aldrich, 0219895525) to promote expression. Relative cell viability was determined at 24 h post-treatment using the RealTime-Glo MT Cell Viability Assay kit (Promega, Catalogue No. G9712) as per the manufacturer's instructions and graphed showing the relative viability measured by relative luminescence units (RLU). The B/B dimerizer was not used for these experiments.

As shown in FIG. 1B, expression of TRIF, and TRIF+RIPK3 reduced cell viability relative to the CT-26-Tet3G parental cell line, confirming the results presented in FIG. 1A. Additionally, induction of TRIF or TRIF+RIPK3 protein expression in the GSDME-expressing cells also reduced cell viability compared to the CT-26-Tet3G parental cells. Together, these results demonstrate that expression of one or more thanotransmission polypeptides, including TRIF, RIPK3 and GSDME, in a cancer cell reduces viability of the cancer cell.

### Example 2. Effects of Cell Turnover Factors (CTFs) from CT-26 Mouse Colon Carcinoma Cells Expressing One or More Thanotransmission Polypeptides on Interferon Stimulated Gene (ISG) Reporters in Macrophages

J774-Dual™ cells (Invivogen, J774-NFIS) were seeded at 100,000 cells/well in a 96-well culture plate. J774-Dual™ cells were derived from the mouse J774.1 macrophage-like cell line by stable integration of two inducible reporter constructs. These cells express a secreted embryonic alkaline phosphatase (SEAP) reporter gene under the control of an IFN-β minimal promoter fused to five copies of an NF-κB transcriptional response element and three copies of the c-Rel binding site. J774-Dual™ cells also express the Lucia luciferase gene, which encodes a secreted luciferase, under the control of an ISG54 minimal promoter in conjunction with five interferon-stimulated response elements (ISREs). As a result, J774-Dual™ cells allow simultaneous study of the NF-κB pathway, by assessing the activity of SEAP, and the interferon regulatory factor (IRF) pathway, by monitoring the activity of Lucia luciferase.

Culture media containing cell turnover factors (CTFs) were generated from CT-26 mouse colon carcinoma cells as described in Example 1 above. In addition to the thanotransmission modules described in Example 1, an additional RIPK3 construct containing a fully Tet-inducible promoter was also evaluated. This Tet-inducible RIPK3 is designated as “RIPK3” in FIG. 2A, and the RIPK3 construct containing the PGK promoter (described in Example 1) is designated as “PGK_RIPK3” in FIG. 2A.

Controls were also included, that would be predicted to induce cell death, without immunostimulatory thanotransmission. These control constructs express i) the C-terminal caspase truncation of human Bid (NM_197966.3), ii) the N-terminal caspase truncation of human GSDMD (NM_001166237.1), iii) a synthetically dimerizable form of human caspase-8 (DmrB-caspase-8), or iv) both DmrB-caspase-8 and human GSDME (NM_004403.3). J774-Dual™ cells were then stimulated for 24 h with the indicated CTFs. Cell culture media were collected, and luciferase activity measured using the QUANTI-Luc (Invivogen; rep-qlcl) assay. Interferon-stimulated response element (ISRE) promotor activation was graphed relative to the control cell line, CT-26-Tet3G.

As shown in FIG. 2A, among the CT-26 cell lines examined, only culture media collected from cells that express TRIF (either alone or in combination with RIPK3) induced ISRE/IRF reporter gene activation in J774-Dual™ cells.

In a separate experiment, the effect of combined expression of Gasdermin E (GSDME) with TRIF or TRIF+RIPK3 was examined. Culture media containing CTFs were generated from the CT-26 cells expressing TRIF or TRIF+RIPK3 as described in Example 1, and in addition from CT-26 cells expressing TRIF+Gasdermin-E or TRIF+RIPK3+Gasdermin-E. As shown in FIG. 2B, culture media from CT-26 cells expressing TRIF (iTRIF), TRIF+RIPK3 (iTRIF_cR3), TRIF+Gasdermin-E (iTRIF_cGE), or TRIF+RIPK3+Gasdermin-E (iTRIF_cR3_cGE) each induced ISRE/IRF reporter gene activation in J774-Dual™ cells. As discussed in Example 1, addition of the dimerizer had little effect on ISRE/IRF reporter gene activation.

Taken together, these results demonstrate that CTFs produced from cancer cells expressing one or more thanotransmission polypeptides activate an immune-stimulatory pathway (i.e. the IRF pathway) in immune cells.

### Example 3. Effects of Cell Turnover Factors (CTFs) from CT-26 Mouse Colon Carcinoma Cells Expressing One or More Thanotransmission Polypeptides on Bone Marrow Derived Dendritic Cells (BMDCs)

Bone marrow cells were differentiated into dendritic cells for 8 days using GM-CSF sufficient RPMI culture medium. 400,000 cells per 2 mL were seeded in a 6-well plate. On day 8, bone marrow derived dendritic cells (BMDCs) were harvested and 100,000 cells/well were seeded in a 96-well plate. BMDCs were then stimulated with media containing CTFs derived from the engineered CT-26 cells described in Example 1. At 24 hours, stimulated cells were harvested and the expression of the cell surface markers CD86, CD40 and PD-L1 was measured by flow cytometry and the mean-fluorescent intensity (MFI) graphed relative to the Tet3G control. Sources of the antibodies were as follows: CD86 (Biolegend, Catalogue No. 105042); CD40 (Biolegend, Catalogue No. 102910); PD-L1 (Biolegend, Catalogue No. 124312). Expression of the cell surface markers CD86, CD40 and PD-L1 is indicative of dendritic cell maturation.

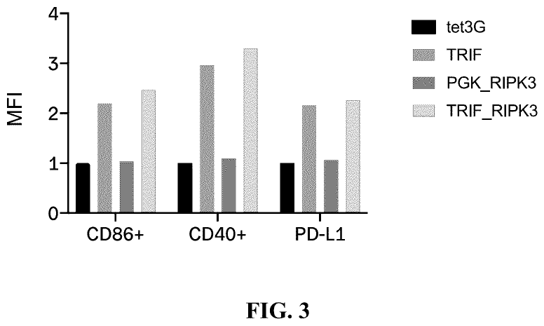

As show in FIG. 3, among the CT-26 cell lines examined, only culture media collected from cells engineered to express TRIF (either alone or in combination with RIPK3) elevated cell surface expression of CD86, CD40, or PD-L1. These results indicate that CTFs from CT-26 cells engineered to express TRIF or both TRIF and RIPK3 induced maturation of the dendritic cells. Upregulation of CD86 and CD40 in the dendritic cells indicates an increased ability to activate T cells. Therefore, the results indicate that CTFs from cancer cells engineered to express TRIF or TRIF and RIPK3 will induce maturation of dendritic cells and increase their ability to activate T cells.

### Example 4. Effect of Thanotransmission Polypeptide Expression Alone or in Combination with Anti-PD1 Antibody on Tumor Growth and Survival in a Mouse Model of Colon Carcinoma

CT-26 mouse colon carcinoma cells harboring the TRIF or TRIF+RIPK3 thanotransmission modules as described in Example 1 were trypsinized and resuspended in serum free media at 1×10⁶ cells/mL. Cells were injected (100 mL) into the right subcutaneous flank of BALB/c mice. From day 11 through day 18 post CT-26 cell injection, regular drinking water was supplemented with doxycycline (Sigma Aldrich, Catalogue No. D9891) at 2 mg/ml to induce thanotransmission polypeptide expression, and from day 11 through day 18, B/B homodimerizer (Takara, Catalogue No. 632622) 2 mg/kg was administered by daily IP injection. Anti-PD1 antibody (BioXcell, Catalogue No. BP0273) and isotype control were administered on day 14, day 17 and day 21. Mice were euthanized when the tumors reached 2000 mm³ in accordance with IACUC guidelines or at the experiment endpoint.

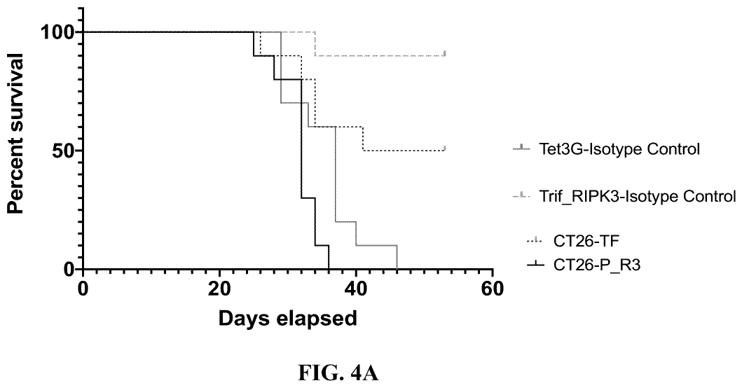

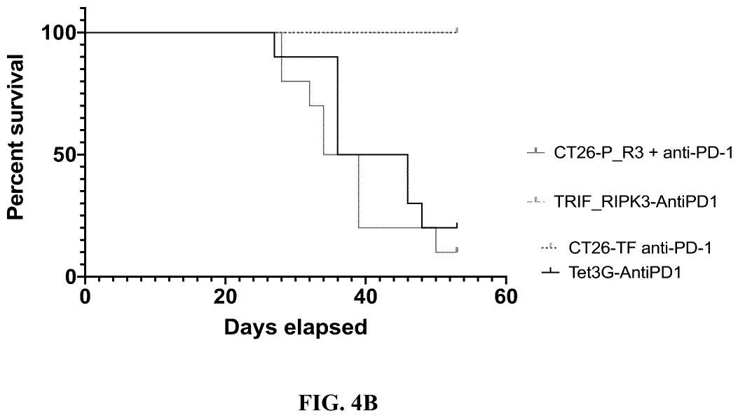

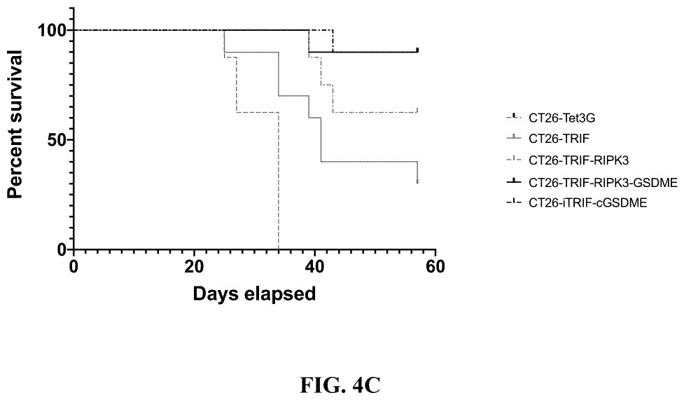

As shown in FIG. 4A, expression of TRIF alone (CT26-TF) increased survival as compared to the CT-26-Tet3G control (Tet3G-Isotype Control) and CT26-RIPK3 cells (CT26-P_R3), and an even greater benefit was observed with the combination of TRIF and RIPK3 (Trif_RIPK3-Isotype Control). As shown in FIG. 4B, the survival of mice injected with CT-26 cells harboring TRIF (CT26-TF) or CT-26 cells harboring TRIF+RIPK3 (TRIF_RIPK3) was enhanced by treatment with anti-PD-1 antibody, with both of these treatment groups exhibiting 100% survival (lines overlapping).

In a separate experiment, CT-26 mouse colon carcinoma cells harboring the TRIF+GSDME and TRIF+RIPK3+GSDME thanotransmission modules described in Example 2 were trypsinized and resuspended in serum free media at 1×10⁶ cells/mL. No B/B homodimerizer was used for this experiment. Cells were injected (100 mL) into the right subcutaneous flank of BALB/c mice. From day 15 through day 21 post CT-26 cell injection, the mice were fed a Teklad base diet supplemented with 625 mg/kg of doxycycline hyclate (Envigo TD.01306). Mice were euthanized when the tumors reached 2000 mm³ in accordance with IACUC guidelines or at the experiment endpoint.

As shown in FIG. 4C, expression of GSDME in combination with TRIF or TRIF+RIPK3 further enhanced survival relative to mice implanted with tumors expressing TRIF alone or TRIF+RIPK3 alone.

### Example 5. Effects of Chemical Caspase Inhibitors on U937 Human Myeloid Leukemia Cells Expressing Thanotransmission Polypeptides

U937 human myeloid leukemia cells and THP1-Dual cells were acquired from ATCC and Invivogen respectively. U937 is a myeloid leukemia cell line. U937 cells expressing human thanotransmission polypeptides (tBid, Caspase 8, RIPK3 or TRIF) were generated using the methods described in Examples 1 and 2, and the doxycycline-inducible expression system described in Example 1.

THP1-Dual cells are a human monocytic cell line that induces reporter proteins upon activation of either NF-kB or IRF pathways. It expresses a secreted embryonic alkaline phosphatase (SEAP) reporter gene driven by an IFN-β minimal promoter fused to five copies of the NF-κB consensus transcriptional response element and three copies of the c-Rel binding site. THP1-Dual cells also feature the Lucia gene, a secreted luciferase reporter gene, under the control of an ISG54 minimal promoter in conjunction with five IFN-stimulated response elements. As a result, THP1-Dual cells allow the simultaneous study of the NF-kB pathway, by monitoring the activity of SEAP, and the IRF pathway, by assessing the activity of a secreted luciferase (Lucia).

To generate conditioned media, 5 million U937-tet3G, U937-tBid, U937-caspase8, U937-RIPK3 or U937-TRIF cells were seeded in a 10 cm dish in RPMI, and subsequently treated for 24 h with doxycycline (1 μg/mL) to induce expression. B/B homodimerizer (100 nM) was added to U937-caspase8, U937-RIPK3 and U937-TRIF cell cultures to promote expression and protein activation via oligomerization. Furthermore, U937-TRIF cells were additionally treated with 4 μM Q-VD-Oph (pan-caspase inhibitor), 10 μM GSK872 (RIPK3 inhibitor) or the combination of both. After cells were incubated for 24 hours, the conditioned media were harvested and sterile filtered.

To measure the thanotransmission polypeptide effect on NF-kB or IRF reporter expression, 100,000 THP1-Dual cells/well were seeded in a 96-well flat-bottom plate in 100 μl volume. 100 μl of conditioned media that generated from U937 cells expressing thanotransmission modules were added to each well. After 24 hour incubation period, 20 μl of THP1-Dual cell culture supernatants were transferred to a flat-bottom 96-well white (opaque) assay plate, and 50 μl of QUANTI-Luc assay solution was added to each well immediately prior to reading luminescence by a plate reader. To measure NF-kB activity, 20 μl of THP1-Dual culture supernatants were transferred to a flat-bottom 96-well clear assay plate, and 180 μl of resuspended QUANTI-Blue solution was added to each well. The plate was incubated at 37° C. for 1 hour and SEAP levels were then measured using a plate reader at 655 nm.

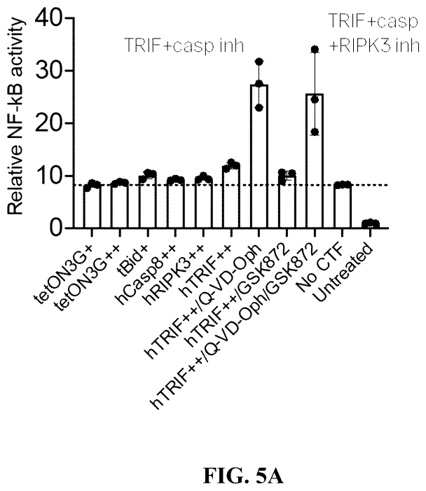

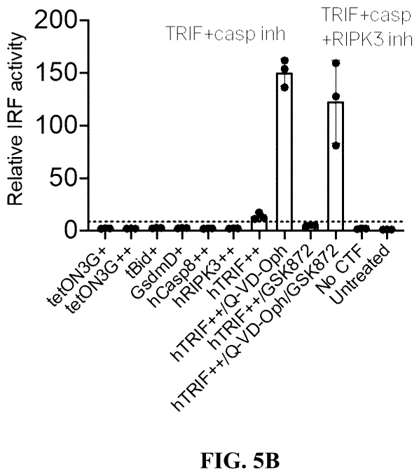

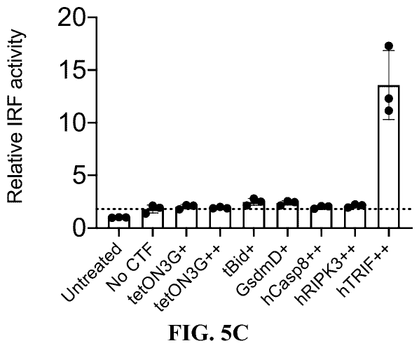

As shown in FIGS. 5A and 5B, treatment of THP-1 Dual cells with cell culture from U937-TRIF cells treated with caspase inhibitor (Q-VD-Oph) alone or in combination with RIPK3 inhibitor (Q-VD-Oph+GSK872) greatly increased NF-kB activation and IRF activity. (In FIGS. 5A-5C, + indicates U937 cells treated with doxycycline, and ++ indicates U937 cells treated with doxycycline and B/B homodimerizer). Cell culture media from U937-TRIF cells treated with RIPK3 inhibitor alone had little effect on NF-kB activation of the THP-1 Dual cells, indicating that the increased NF-kB activation was due to caspase inhibition. As shown in FIGS. 5B and 5C, treatment of THP-1 Dual cells with cell culture media from U937-TRIF cells that were not treated with caspase inhibitor also increased IRF activity, although to a lesser extent than U937-TRIF cells treated with caspase inhibitor.

Taken together, these results demonstrate that CTFs produced from human cancer cells expressing TRIF activate immune-stimulatory pathways (i.e. the NF-kB and IRF pathways) in immune cells, and that caspase inhibition enhances this effect.

### Example 6. Modulation of Thanotransmission in CT-26 Mouse Colon Carcinoma Cells by Expressing Combinatorial Thanotransmission Polypeptides Including Caspase Inhibitor Proteins

The experiment described in this example tested the effect of expression of caspase inhibitor proteins on thanotransmission in cancer cells expressing TRIF and RIPK3.

CT26 mouse colon carcinoma cells expressing the thanotransmission polypeptides TRIF and RIPK3, as described in Example 1, were transduced with genes encoding: (i) a dominant negative version of human Fas-associated protein with death domain (FADD; Accession No. NM_003824); (ii) the short version of human cellular FLICE-like inhibitory protein (cFLIPs; Accession No. NM_001127184.4); or (iii) viral inhibitor of Caspase (vICA, HCMV gene UL36; Accession No. NC_006273.2) in order to modulate thanotransmission by inhibiting caspase activity. FADD-DN, cFLIPs and vICA were each cloned into the pLV-EF1a-MCS-IRES-Puro vector (Biosettia), and used to transduce CT26-TRIF-RIPK3 expressing cells.

These cells were seeded and subsequently treated for 24 h with doxycycline (1 mg/mL; Sigma Aldrich, 0219895525) to promote expression. B/B homodimerizer was not used in this experiment. Relative cell viability was determined at 24 h post-treatment using the RealTime-Glo MT Cell Viability Assay kit (Promega, Catalogue No. G9712) as per the manufacturer's instructions and graphed showing the relative viability measured by relative luminescence units (RLU).

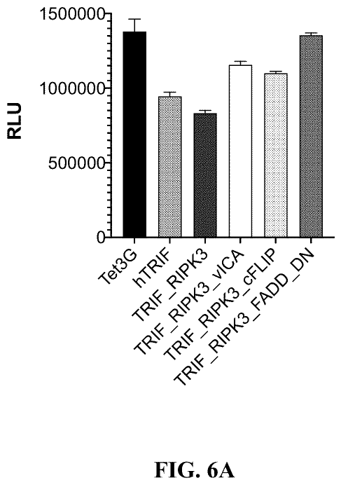

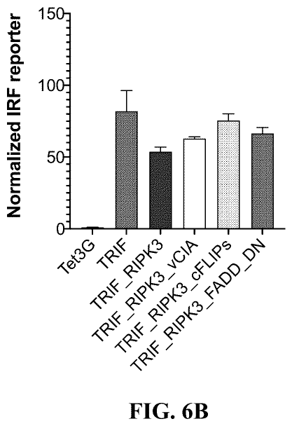

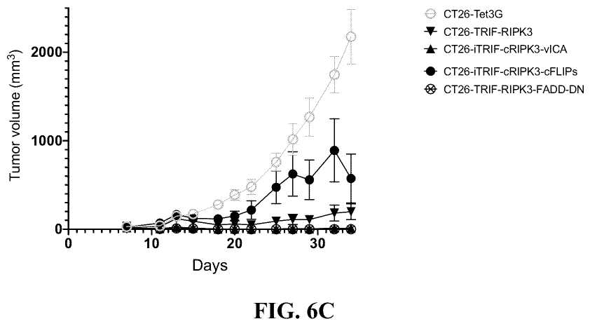

As shown in FIG. 6A, expression of any one of FADD-DN, cFLIPs or vICA in the CT26-TRIF+RIPK3 cells attenuated the decrease in cancer cell viability induced by TRIF+RIPK3 expression. However, expression of cFLIPs+TRIF+RIPK3 or vICA+TRIF+RIPK3 in CT26 cells still reduced cancer cell viability relative to the parental line CT26-Tet3G cell line, just to a lesser extent than TRIF-RIPK3 alone. See FIG. 6A.

Next, culture media containing CTFs were generated from CT-26 mouse colon carcinoma cells as described above in Example 5. J774-Dual™ cells were then stimulated for 24 h with the indicated CTFs. Cell culture media were collected, and luciferase activity measured using the QUANTI-Luc (Invivogen; rep-qlcl) assay. Interferon-stimulated response element (ISRE) promotor activation was graphed relative to the control cell line, Tet3G. As shown in FIG. 6B, media collected from CT26 cell lines expressing TRIF or TRIF+RIPK3 induced IRF reporter expression in J774-Dual cells. In addition, media from CT26 cells expressing FADD-DN, cFLIPs or vICA in addition to TRIF+RIPK3 also induced IRF reporter activation in J774-Dual cells.

CT-26-TRIF+RIPK3 mouse colon carcinoma cells harboring the FADD-DN, cFLIPs or vICA thanotransmission modules described above were trypsinized and resuspended in serum free media at 1×10⁶ cells/mL. No B/B homodimerizer was used in this experiment. Cells were injected (100 μL) into the right subcutaneous flank of immune-competent BALB/c mice. From day 15 through day 21 post CT-26 cell injection, the mice were fed a Teklad base diet supplemented with 625 mg/kg of doxycycline hyclate (Envigo TD.01306). Mice were euthanized when the tumors reached 2000 mm³ in accordance with IACUC guidelines or at the experiment endpoint.

As shown in FIG. 6C, growth of all tumors expressing a thanotransmission module (i.e. TRIF+RIPK3, TRIF+RIPK3+FADD-DN, TRIF+RIPK3+cFLIPS, or TRIF+RIPK3+vICA) was reduced relative to control CT26-Tet3G cells. In particular, expression of FADD-DN or vICA in combination with TRIF+RIPK3 further reduced tumor growth, as compared to the parental CT26-TRIF+RIPK3 cells. Interestingly, although the thanotransmission modules comprising FADD-DN or vICA in addition to TRIF+RIPK3 were most effective in reducing tumor growth in vivo, the FADD-DN+TRIF+RIPK3 had little effect on CT26 cancer cell viability in vitro relative to the TRIF+RIPK3 cells, while vICA+TRIF+RIPK3 coexpression enhanced cell killing in vitro relative to TRIF+RIPK3. These results suggest that in addition to the magnitude of cancer cell killing by thanotransmission modules, the precise cell turnover factor (CTF) profile produced by the cancer cells due to expression of these modules may also contribute to the immune response to the tumor cells in vivo.

### Example 7. Effects of Human TRIF Variants on Cancer Cell Viability and IRF and NFkB Activity In Vitro

The aim of this study was to determine the effects of different variants of the human TRIF protein on viability of human cancer cell lines and induction of the IRF and NFkB pathway in a human monocyte line.

To test the constructs, the sequences for the human TRIF variants were designed, codon-optimized, synthesized and cloned into a commercial lentiviral vector (pLVX-TetONE-Puro, Takara). Two control cell lines were also prepared, TETON3G and tBID. The TetON3G cell line is a negative control that expresses a transactivator for doxycycline induction without heterologous expression of a cell death inducer gene. The tBID cell line is a positive control for cytotoxicity, where tBID is expressed in a doxycycline-independent manner. The TRIF constructs also contained a FLAG-tag at the C-terminus for monitoring of protein expression. The TRIF constructs were transduced into human colorectal adenocarcinoma HT29 cells and human melanoma A375 cells. Expression of TRIF constructs was induced through a doxycycline-inducible system (TetON system, Takara). After one day of doxycycline treatment, cell viability was assessed using the CellTiterGlo2.0 assay (“CTG”, Promega) and the supernatant was transferred onto THP1-Dual monocytes (Invivogen). The THP1-Dual monocytes contained two reporters for IRF and NFkB activity using a secreted luciferase and a secreted alkaline phosphatase, respectively. The TRIF constructs evaluated in this study are described in Table 4 below.

TABLE 4
Human TRIF Constructs

| | | Nucleic | Amino |
|---|---|---|---|
| | | Acid | Acid |
| Name | Description | SEQ ID NO: | SEQ ID NO: |
| TRIF WT | Wildtype full-length human TRIF | 1 | 2 |
| TRIF_mutRHIM | Mutation of the RHIM tetrad of TRIF into | 3 | 4 |
| | AAAA (aa688-691 - QLGL to AAAA) | | |
| TRIF_Trunc | Truncation of the C-terminal fragment (541- | 5 | 6 |
| | 712) of TRIF containing the RHIM domain | | |
| TRIF_PhosphoM | Mutations of the TRIF TBK1 phosphorylation | 7 | 8 |
| | sites (S210A, S212A, T214A). | | |
| | Phosphorylation of TRIF at these residues by | | |
| | TBK1 enables the recruitment of IRF3 and its | | |
| | activation. | | |
| TRIF_P434H | Mutation for dimerization site P434 in the | 9 | 10 |
| | TIR domain of TRIF | | |
| miniTRIF | N-terminal deletion (1-311) of TRIF | 11 | 12 |
| TRIF_d1-180 | N-terminal deletion (1-180) of TRIF | 13 | 14 |
| TIR domain | TIR domain of TRIF alone | 15 | 16 |
| TRIS | Deletion of N-terminal fragment 1-180 and | 17 | 18 |
| | fragment 217-658 of TRIF | | |
| TRIR | Deletion of N-terminal fragment 1-180, | 19 | 20 |
| | fragment 217-386 and fragment 546-712 of TRIF | | |
| TRIR3 | TRIR followed by a flexible linker GPGGSSGSS | 21 | 22 |
| | and hRIPK3 (UniProtKB - Q9Y572 (RIPK3_HUMAN)) | | |

### Results.

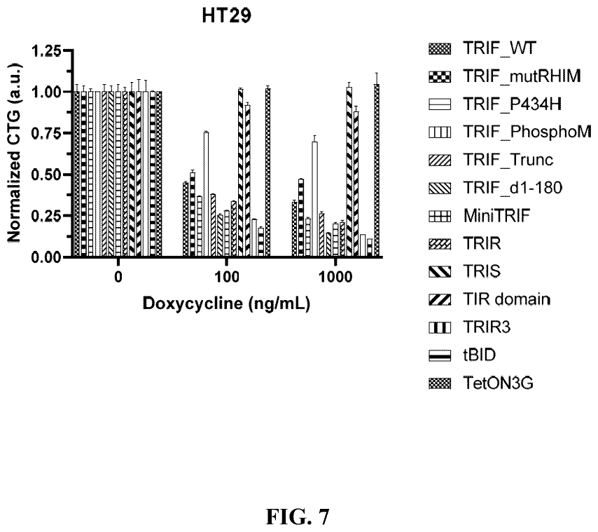

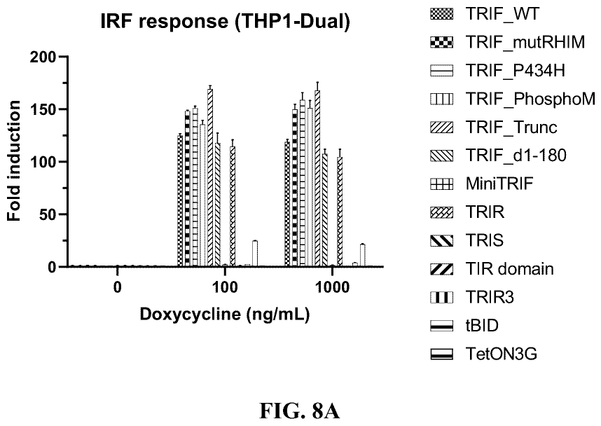

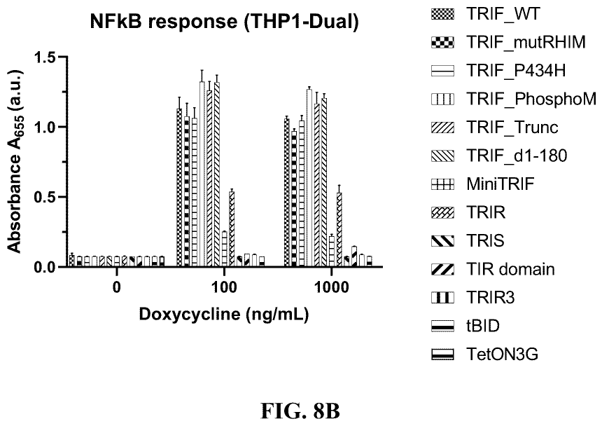

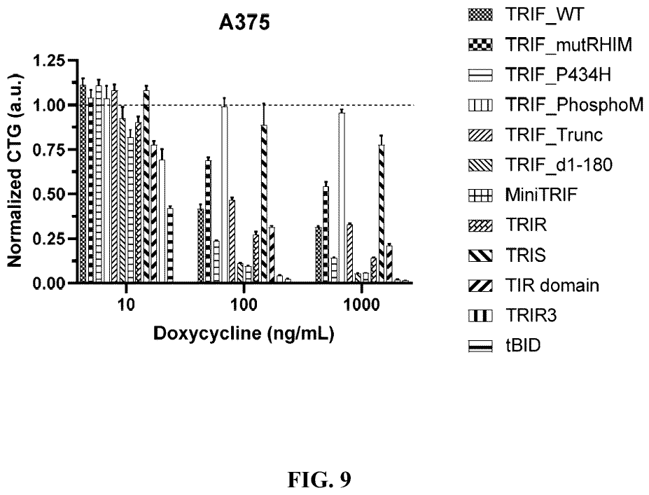

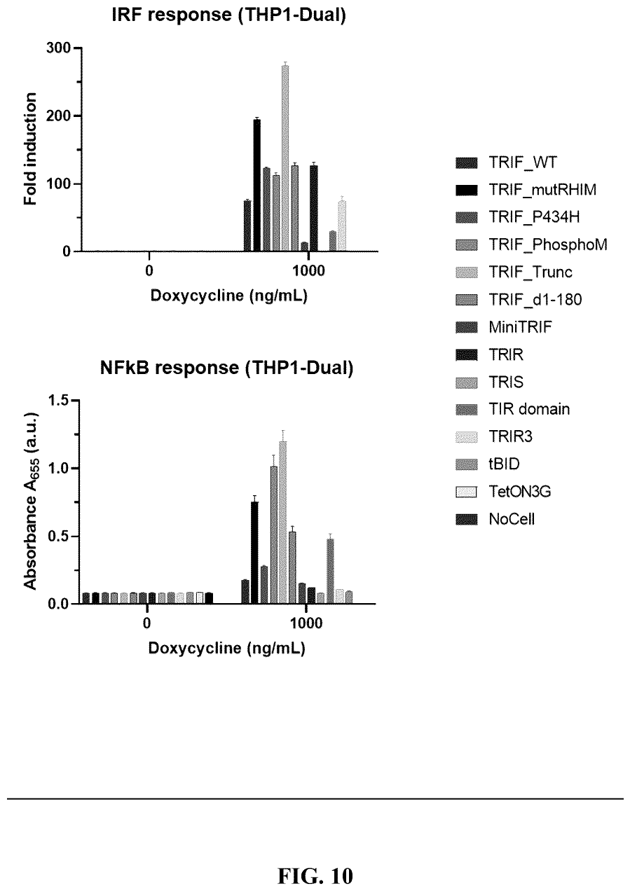

As shown in FIGS. 7 and 9, expression of the TIR domain alone (“TIR domain” construct) had some cytolytic activity, and this cytolytic activity was enhanced by adding the TRIF fragment aa181-aa216 to the TIR domain (“TRIR” construct). Unexpectedly, the RHIM domain was not necessary for cytolytic activity, as shown by deletion of the RHIM tetrad (“TRIF_mutRHIM” construct) or by truncation of the C-terminal region of TRIF (“TRIF_Trunc” construct), which contains the RHIM domain. Expression of a TRIF variant with mutations of three amino acid residues phosphorylated by TBK1 (“TRIF_PhosphoM” construct) showed little cytotoxic activity in HT29 cells, and no cytotoxic activity in A375 cells. Unexpectedly, this TRIF variant maintains its IRF and NFkB activity, suggesting a potential uncoupling of cell death and activation of those pathways. This result is reinforced by the IRF assay in HT29 cells (see FIG. 8), where cell death and IRF activation can be uncoupled depending on the variants at low dose of doxycycline. Expression of the miniTRIF variant is highly potent for cell death (see FIGS. 7 and 9), but leads to a low induction of the NFkB and IRF pathways in the THP1-Dual assay (see FIGS. 8 and 10). Addition of the fragment aal81-aa310 to miniTRIF (“TRIF_d1-180” construct) strongly activates the NFkB and IRF pathways, while still maintaining high potency for cell death. Expression of the TRIF variant consisting of a fusion between TRIR and human RIPK3 (“TRIR3” construct) can induce cell death and activate the IRF and NFkB pathways in THP1-Dual cells at a lower level than TRIF or TRIR alone. Expression of the TRIS variant, containing the fragment aa181-aa216 with the RHIM domain, did not lead to cell death or activation of THP1-Dual cells, contrary to previous published findings (Han et al., JBC 285:12543-12550 (2010)).

Taken together, these results indicate that i) the TIR domain appears to be the minimal fragment for inducing cell death, while other domains of the protein (e.g., the RHIM domain) can diversify the modes and magnitude of cell death; ii) the TBK1 phosphorylation sites of TRIF (S210A, S212A and T214A) are important contributors to cell death, especially in A375 cells; iii) the combination of the aal81-aa216 fragment and TIR domain results in a high level of IRF induction in the THP1-Dual assay; iv) the fusion construct containing the TIR domain of the TRIF protein and the RIPK3 protein (“TRIR3”) possesses unexpected features of high cytotoxicity, moderate IRF induction and low/absent NFkB induction.

### Example 8. Evaluation of Combinatorial Thanotransmission Polypeptides Including miniTRIF in a Mouse Model of Colon Cancer

CT26 mouse colon carcinoma cells expressing the Tet-On 3G transactivator for doxycycline-inducible expression as described in Example 1, were transduced with genes encoding: (i) miniTRIF (see Table 4)+mouse RIPK3 (Example 1, Accession No.: NM_019955.2); or ii) miniTRIF+GSDME (Accession No.: NM_001127453.2). CT26 cells expressing Tet-On 3G activator alone, miniTRIF+RIPK3 (+Tet-On 3G activator), or MiniTRIF+GSDME (+Tet-On 3G activator) modules as described above were trypsinized and resuspended in serum free media at 1×10⁶ cells/mL. No B/B homodimerizer was used in this experiment. Cells (100 μL) were injected into the right subcutaneous flank of immune-competent BALB/c mice. From day 13 through day 19 post CT-26 cell injection, the mice were fed a Teklad base diet supplemented with 625 mg/kg of doxycycline hyclate (Envigo TD.01306). Mice were euthanized when the tumors reached 2000 mm³ in accordance with IACUC guidelines or at the experiment endpoint.

### Results

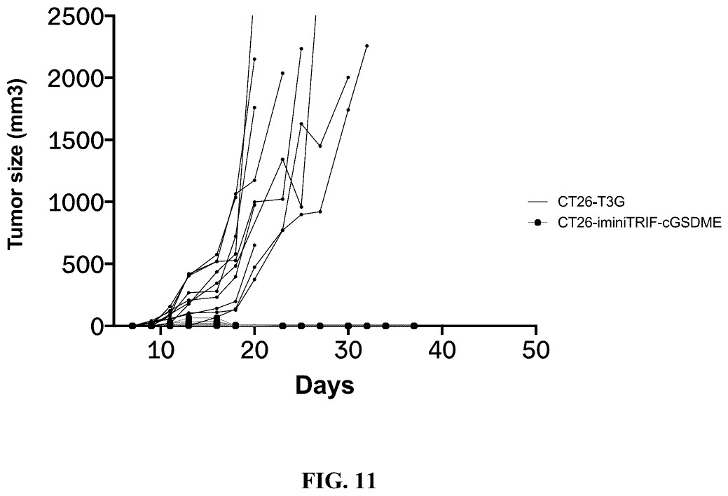

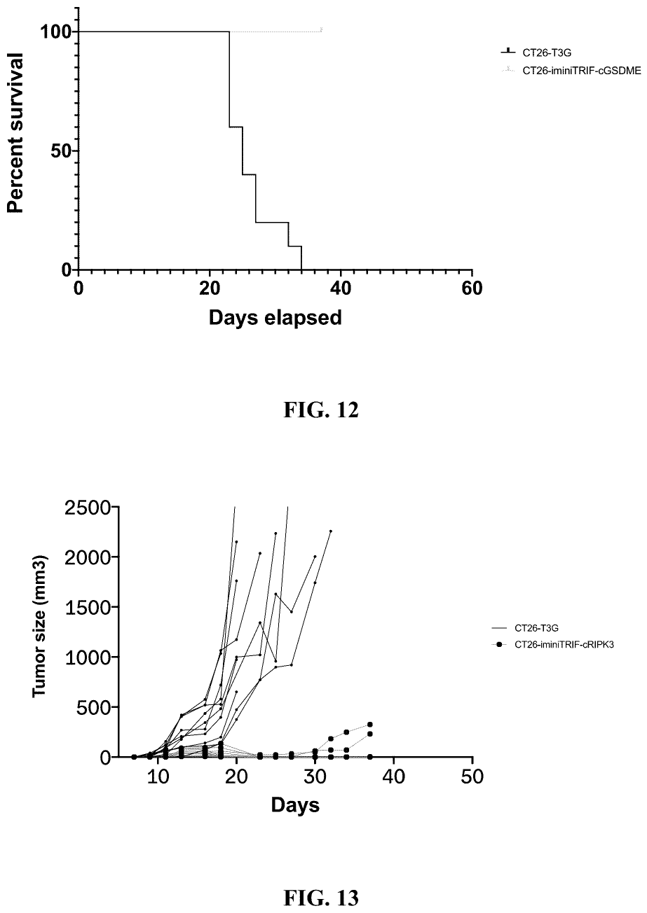

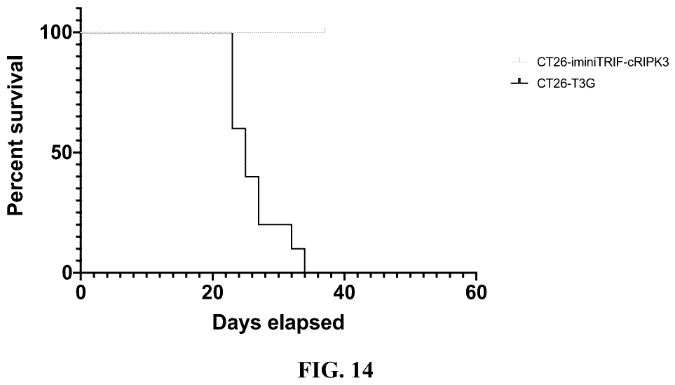

As shown in FIG. 11, the combination of miniTRIF and GSDME greatly reduced tumor size relative to the control. The combination of miniTRIF and GSDME also greatly increased survival relative to the control, as shown in FIG. 12. For example, all of the mice containing CT26 cells expressing the combination of miniTRIF and GSDME were still alive on Day 34, while all of the control animals were dead. The combination of miniTRIF and RIPK3 also greatly reduced tumor size, and greatly increased survival, as shown in FIGS. 13 and 14.

Taken together, these results indicate that expression of thanotransmission polypeptides in cancer cells can reduce tumor growth and increase survival.

### Example 9. Effects of RIPK3, TRIF and vICA Expressed from Adenovirus on Cancer Cell Viability and IRF Activity In Vitro

The aim of this study was to determine the effects of RIPK3 alone or in combination with TRIF or TRIF+vICA expressed from replication incompetent adenovirus 5 on viability of human cancer cell lines and induction of the IRF pathway in J774-Dual™ cells (Invivogen, J774-NFIS)

J774-Dual™ cells were derived from the mouse J774.1 macrophage-like cell line by stable integration of inducible reporter constructs. For example, these cells express the Lucia luciferase gene, which encodes a secreted luciferase, under the control of an ISG54 minimal promoter in conjunction with five interferon-stimulated response elements (ISREs). As a result, J774-Dual™ cells allow study of the interferon regulatory factor (IRF) pathway by monitoring the activity of Lucia luciferase.

This experiment tested the effects of replication incompetent adenovirus 5 (E1 and E3 region deleted) expressing mouse RIPK3 (mRIPK3), human TRIF-P2A-mRIPK3, or human TRIF-P2A-mRIPK3-P2A-vICA on thanotransmission in the wild type mouse cancer cell lines 4T1 (breast cancer), MC38 (colon cancer) and Pan02 (pancreatic cancer). Mouse RIPK3 was used in the mouse model because RIPK3 has species specificity. Because TRIF does not have species specificity, human TRIF was used. The P2A peptide induces ribosomal skipping during translation, such that TRIF, mRIPK3 and vICA were expressed as separate proteins.

The cancer cell lines 4T1, MC38 and Pan02 were seeded and subsequently treated with adenovirus 5 expressing mock control, mRIPK3, TRIF-P2A-mRIPK3, or TRIF-P2A-mRIPK3-P2A-vICA for 72 hours at a multiplicity of infection (MOI) of 50 (i.e., 50 adenoviruses per cancer cell). The cell death was measured using SYTOX green (Life technologies; 57020) and total cell numbers were measured by nuclei staining with Hoechst (Fisher Scientific; H1399).

Conditioned culture media containing cell turnover factors (CTFs) were generated from the cells treated with the above recombinant adenoviruses essentially as described in Example 5, by culturing the cells for 48 hours after treatment with the adenovirus. After the 48 hour incubation, the conditioned media was harvested and sterile filtered. For measurement of IRF activity, J774-Dual™ cells were then stimulated for 24 h with the indicated CTFs. Specifically, 100,000 J774-Dual cells/well were seeded in a 96-well flat-bottom plate in 100 μl volume. 100 μl of conditioned media that was generated from the various cancer cells expressing thanotransmission modules were added to each well. Cell culture media from the J774-Dual™ cells were collected, and luciferase activity measured using the QUANTI-Luc (Invivogen; rep-qlcl) assay. Interferon-stimulated response element (ISRE) promotor activation was graphed relative to the control cell line, Tet3G to show IRF activity.

Results

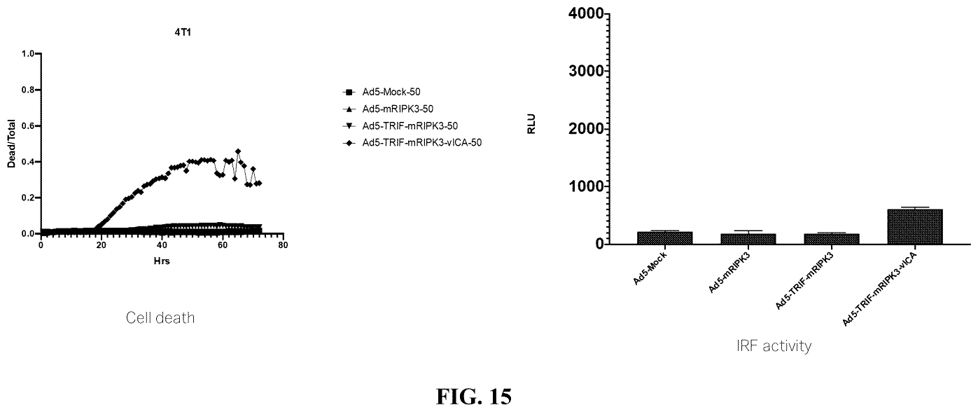

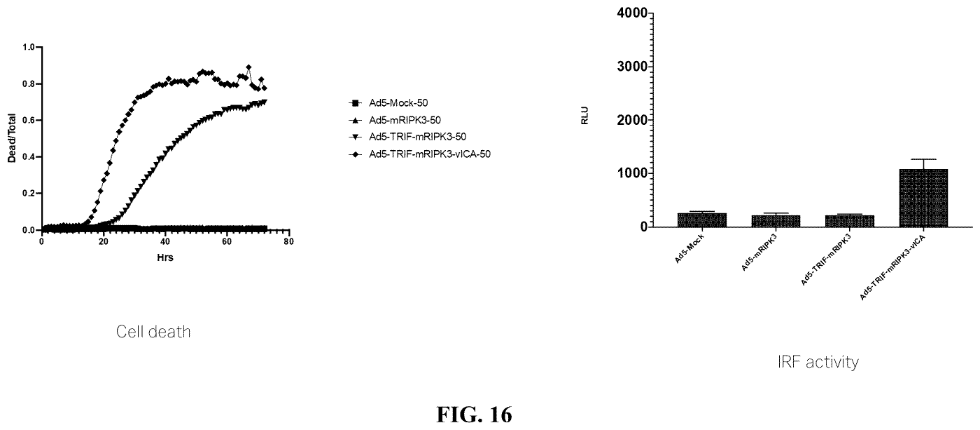

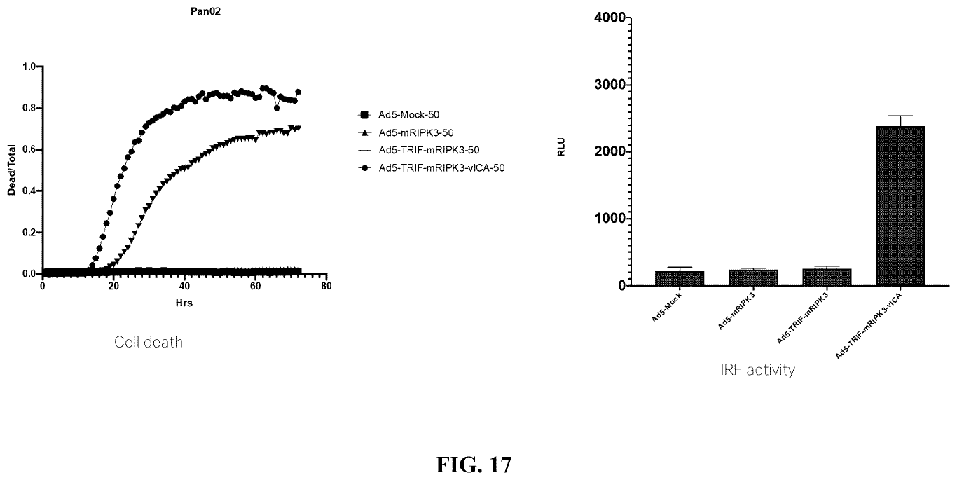

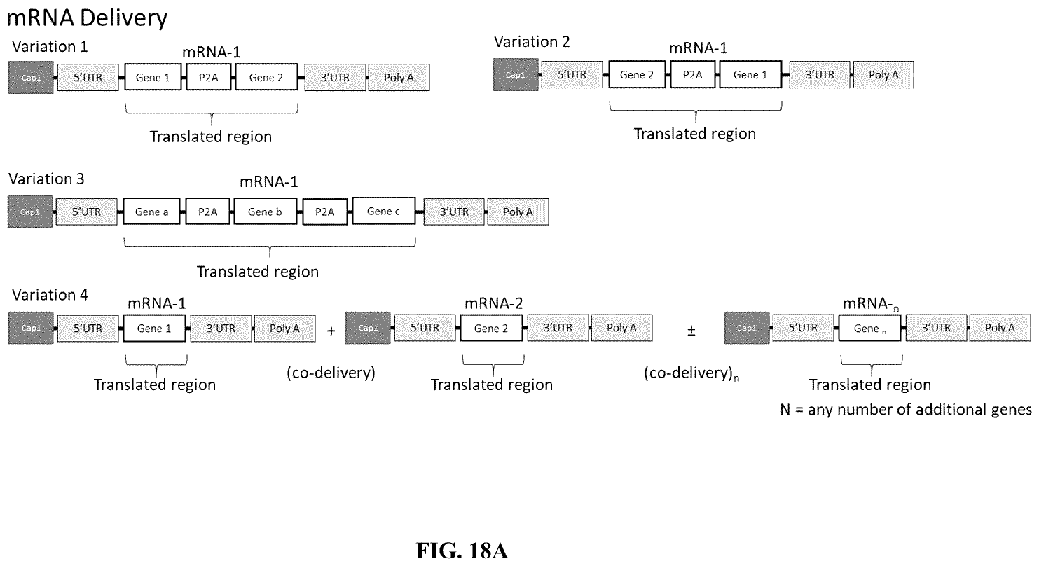

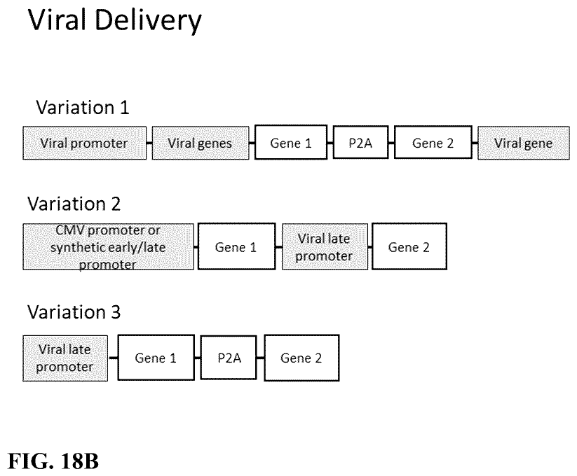

FIGS. 15-17 show cell death relative to viable cells over 72 hrs. As shown in FIG. 15 left panel, the combination of TRIF+mRIPK3+vICA greatly increased cell death in 4T1 mouse breast cancer cells, while the mRIPK3 alone, TRIF+mRIPK3, and mock control exhibited little or no cell death. As shown in FIGS. 16 and 17, both TRIF+mRIPK3+vICA and TRIF+mRIPK3 greatly increased cell death in MC38 colon cancer cells (FIG. 16, left panel) and Pan02 pancreatic cancer cells (FIG. 17, left panel) relative to mRIPK3 alone and the mock control, with TRIF+mRIPK3+vICA expression resulting in higher and more rapid cell death.

As shown in FIGS. 15-17, right panels, the media collected from 4T1, MC38 and Pan02 cells expressing TRIF+mRIPK3+vICA strongly induced IRF reporter expression in J774-Dual™ cells, while expression of mRIPK3 alone and mRIPK3+TRIF had little effect on IRF activity.

This latter result for medium collected from the MC38, 4T1 and Pan02 cells treated with Ad5-hTRIF-P2A-mRIPK3 is different from the dox inducible thanoswitch results described above where medium collected from CT26 cells that expressed hTRIF and mRIPK3 induced IRF activity in J774 cells. While the reason for this difference remains to be characterized, the lack of IRF activity in the adenovirus expression system may be due to lower RIPK3 and TRIF expression levels relative to the dox inducible system. Alternatively, it is possible that the dose of infectious adenovirus particles in this experiment was not sufficient for mRIPK3+TRIF to induce IRF activity.

### Example 10. Identification of Thanotransmission Polypeptides and Combinations Thereof Through Clustering Analysis

A series of genes encoding thanotransmission polypeptides were identified for their potential ability to modulate microenvironments, immune induction, and effects on cell health. These genes were strategically paired into unique combinations and expressed in target cells. The cell microenvironment was studied by identifying factors being released by the cells upon expression of the thanotransmission polypeptides and assays were performed to detect the downstream effect on target and responder cells. This data was aggregated, and unsupervised clustering analysis was performed, identifying combinations of thanotransmission polypeptides with desirable activity. Function was confirmed through in vitro and in vivo studies described herein for at least a representative number of thanotransmission polypeptides and combinations thereof. The combinations of TRIF and one or more additional thanotransmission polypeptides described herein, and the CASP1FV and TRIF_180 variants, were selected based on this analysis.

### Example 11. Cell Viability and Cytokine/Chemokine Secretion of Cancer Cells Transfected with mRNA Encoding TRIF, TRIF+RIPK3, TRIF+RIPK3+vICA, or TRIF+GSDME

### Methods

Each mRNA prepared encoded a single thanotransmission polypeptide, i.e., TRIF, RIPK3, vICA or GSDME. All of the mRNAs used in these experiments contained (from 5′ to 3′) a 5′ cap structure, a 5′ UTR, a thanotransmission polypeptide coding region (i.e., TRIF, RIPK3, vICA or GSDME), a 3′ UTR, and a polyA region. The mRNAs encoding TRIF, RIPK3, vICA or GSDME were in vitro transcribed in an enzymatic reaction containing an RNA polymerase, dNTPs, inorganic phosphatase, RNase inhibitor, and a plasmid DNA template. The plasmid DNA template, which contained the mRNA sequence cassette including a RNA polymerase promoter and a polyA tract of 120 As, was linearized with a restriction enzyme which cut into the polyA tract. The in vitro transcription reaction was stopped with the addition of DNAse, and the mRNA was purified from the reaction mixture. In a subsequent reaction, a 5′ Cap1 structure was added to the mRNA using a capping enzyme and methyltransferase (the methyl donor was SAM-TOS). This final mRNA was purified from the reaction mixture.

CT26 murine colorectal carcinoma cells, B16F10 murine melanoma cells, and MC38 murine colon adenocarcinoma cells were plated at the manufactures recommended density for transfection using the jet Messenger in vitro transfection kit. The mRNAs encoding a single thanotransmission polypeptide were combined to test combinations of the thanotransmission polypeptides. The treatment groups tested are shown below.
1. TRIF
2. TRIF+RIPK3
3. TRIF+RIPK3+vICA
4. TRIF+GSDME
5. No mRNA (negative control).

The cells were transfected with mRNA(s) encoding TRIF, TRIF+RIPK3,

TRIF+RIPK3+vICA, or TRIF+GSDME at a gene ratio of 1:1 or 1:1:1 with the total mRNA equal to 0.1 μg/well. Accordingly, the copy number of TRIF was higher in the TRIF alone treatment group (Group 1) relative to the other treatment groups testing combinations of thanotransmission polypeptides (Groups 2-4). Cell viability was monitored for 48 h using a Cytation 5 instrument.

For determination of cytokine/chemokine secretion, cancer cell culture supernatants were collected at 24 h following transfection, and the production of various cytokines and chemokines were determined using Biolegend's mouse Legendplex kits for Antiviral responses and Pro-inflammatory Chemokines. Data was determined as the fold induction over the negative control and plotted on a log 2 scale.

### Results

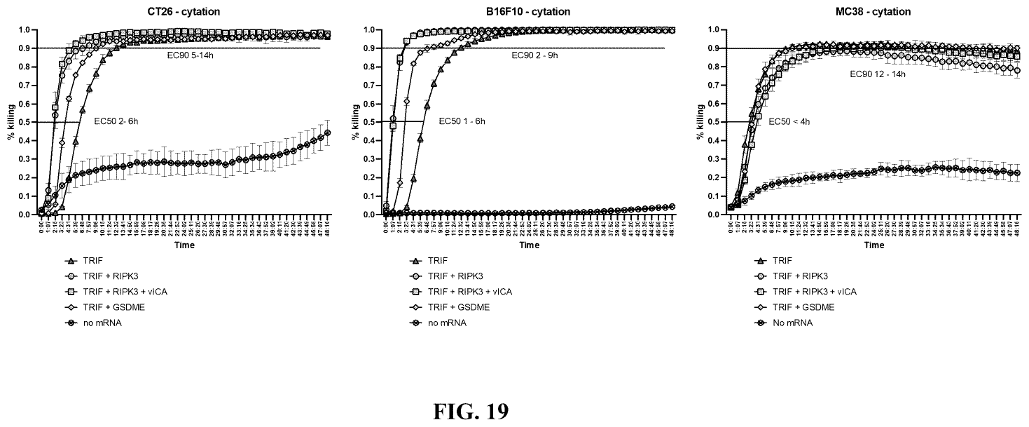

As shown in FIG. 19, for both the CT26 murine colorectal carcinoma cells and B16F10 murine melanoma cells, transfection with mRNA encoding TRIF+RIPK3 or TRIF+RIPK3+vICA resulted in the greatest amounts of cell killing, followed by transfection with mRNA encoding TRIF+GSDME. Transfection with mRNA encoding TRIF alone increased cancer cell killing relative to the no mRNA negative control, but to a lesser extent than the combination with GSDME, RIPK3 or RIPK3+vICA. For the MC38 murine colon adenocarcinomac cells, transfection with mRNA encoding TRIF, TRIF+GSDME, TRIF+RIPK3 or TRIF+RIPK3+vICA resulted in similar levels of cell killing, and all of these treatment groups exhibited increased cell killing relative to the no mRNA negative control.

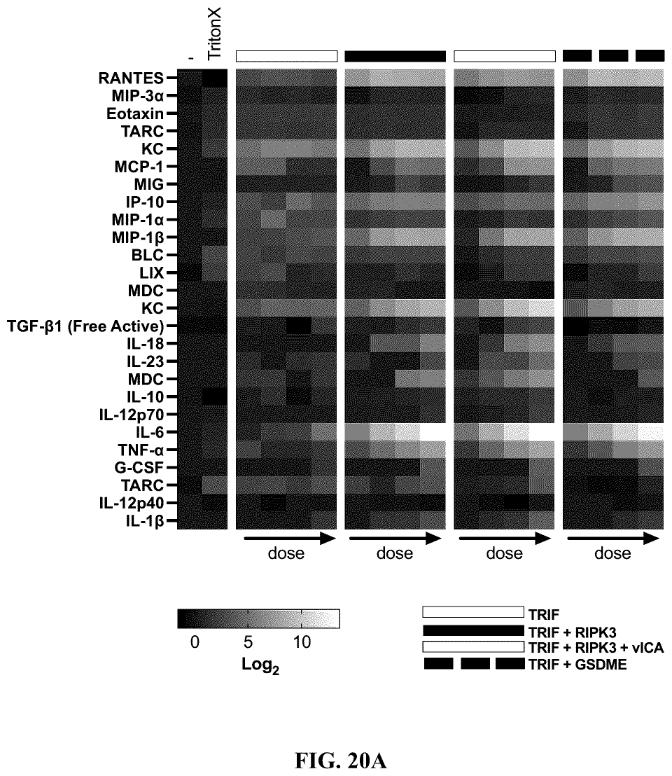

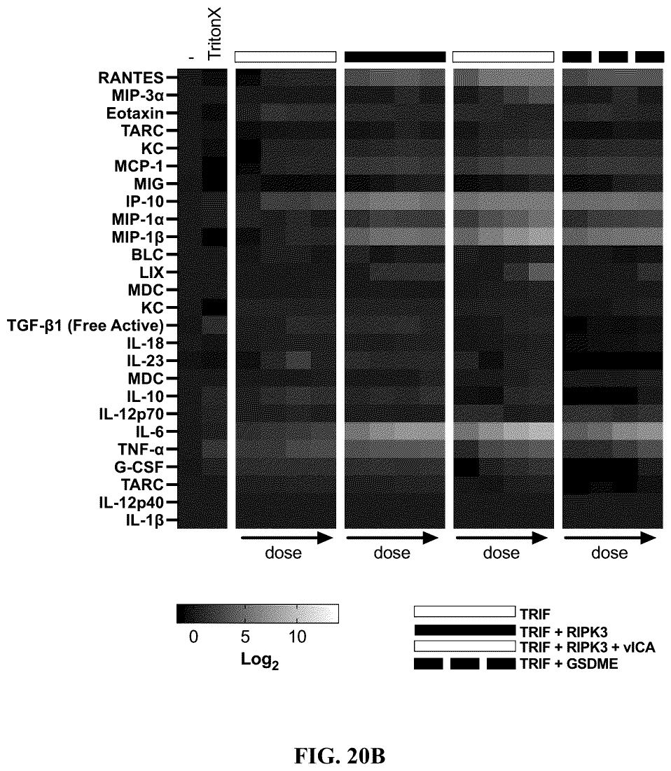

As shown in FIGS. 20A and 20B, transfection with mRNA encoding TRIF+GSDME, TRIF+RIPK3 or TRIF+RIPK3+vICA increased cytokine and chemokine secretion relative to TRIF alone in B16F10 cells (20A) and MC38 cells (20B).

### Conclusions

The cell killing results demonstrate that transfection of cancer cells with mRNA encoding TRIF is effective in killing cancer cells, and combination of TRIF with GSDME, RIPK3 or RIPK3+vICA is even more effective than TRIF alone in killing cancer cells.

The cytokine/chemokine secretion results demonstrate that transfection of cancer cells with mRNA encoding TRIF+GSDME, TRIF+RIPK3 or TRIF+RIPK3+vICA increased secretion of immunomodulatory proteins to a greater extent than mRNA encoding TRIF alone, suggesting that these combination treatments would increase immune activity to a greater extent than TRIF alone.

### Example 12. Evaluation of Intratumoral Injection of mRNAs Encoding Combinatorial Thanotransmission Polypeptides in a Mouse Model of Melanoma

### Methods

Female C57BL/6 mice were implanted with B16F10 murine melanoma cells in the rear-right hind flank and tumors were allowed to grow to a size of ˜100 mm³. At 100 mm³, mice were randomized, and mRNA and control treatments were administered intratumorally at 24 hour intervals on Day 0, 1, 2 and 3. Each mRNA encoded a single thanotransmission polypeptide (i.e., TRIF, RIPK3, vICA or GSDME) and were prepared as described above in Example 11. For each of the four injections, each mouse received a total of 8 μg of LNP-formulated mRNA encoding TRIF+RIPK3, TRIF+RIPK3+vICA, TRIF+GSDME, or firefly luciferase (as a negative control). For the mRNAs encoding combinations of thanotransmission polypeptides, the genes were administered at a ratio of 1:1 (i.e., TRIF:RIPK3 or TRIF:GSDME) or 1:1:1 (i.e., TRIF:RIPK3:vICA). Since the total weight of the mRNA administered was the same for each treatment group, the copy number of the TRIF and RIPK3 genes was higher in the TRIF+RIPK3 treatment group relative to the TRIF+RIPK3+vICA treatment group. An additional treatment group received an equivalent volume of PBS as a further negative control.

### Results

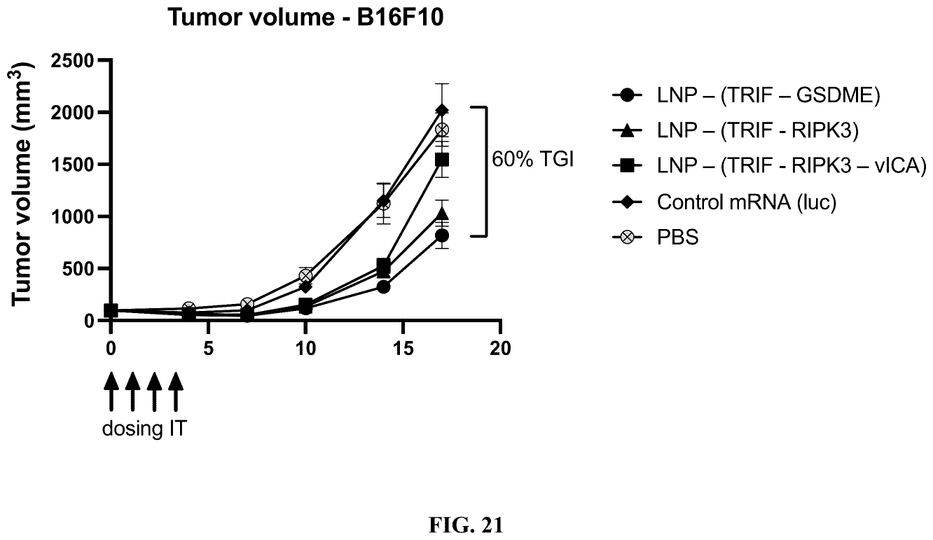

As shown in FIG. 21, intratumoral injection of mRNAs encoding TRIF+GSDME TRIF+RIPK3, or TRIF+RIPK3+vICA reduced tumor growth relative to the PBS and firefly luciferase negative controls, with the greatest tumor growth inhibition observed for TRIF+GSDME and TRIF+RIPK3. For example, on Day 17, mRNA encoding TRIF+GSDME or TRIF+RIPK3 reduced tumor growth approximately 60% relative to the negative controls. The increased efficacy of the TRIF+RIPK3 treatment in inhibiting tumor growth on Day 17 relative to the TRIF+RIPK3+vICA may be due to the higher copy number of the TRIF and/or RIPK3 genes in the TRIF+RIPK3 treatment, as indicated above in the methods section.

### Conclusion

These results demonstrate that treatment of tumors with mRNAs encoding thanotransmission polypeptides is effective in reducing tumor growth in vivo.

### Example 13. Evaluation of Colorectal Carcinoma Tumors Expressing CASP1FV, TRIF_180, TRIF+Npro or TRIF+GSDME In Vivo

### Methods

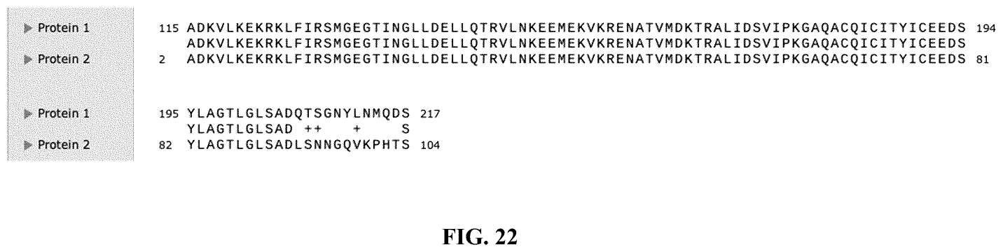

A series of murine colorectal carcinoma CT26 tumors were generated that expressed a caspase 1 variant (CASPlFV), a TRIF variant (TRIF_180), TRIF+Npro or TRIF+GSDME under a doxycycline (Dox) inducible promoter. A CT26 cell line was transduced with a lentiviral vector encoding, CASP1FV, TRIF_180, TRIF+Npro or TRIF+GSDME. For the TRIF+Npro and TRIF+GSDME vectors, each thanotransmission polypeptide was separated by a P2A element to allow for translation of individual polypeptides. The CASP1FV variant comprises a self-dimerization domain at its C-terminus. See FIG. 22, Protein 2. TRIF_180 is human TRIF comprising a deletion of amino acid residues 1-180. As a negative control, CT26 cells were also transduced with empty Tet3G vector. This negative control allowed for determination of any Dox induced anti-tumor effects. Six-week-old female BALB/c mice were inoculated with tumor cells contain dox-inducible thanotransmission polypeptides in the right flank. The Tet3G negative control tumors were inoculated at a concentration of 1e⁵ cells/100 μl, while the other treatment groups were inoculated at a concentration of 5e⁵ cells/100 uL. Mice were randomized when tumors reached 100 mm³ and divided equally into two groups, with one group given normal mouse chow, and the other normal mouse chow containing Dox, for 17 days. Tumor growth was monitored in mice that were exposed to Dox and compared to genetically matched tumors that were not exposed to Dox (shown in the figures as On Dox vs Off Dox).

### Results

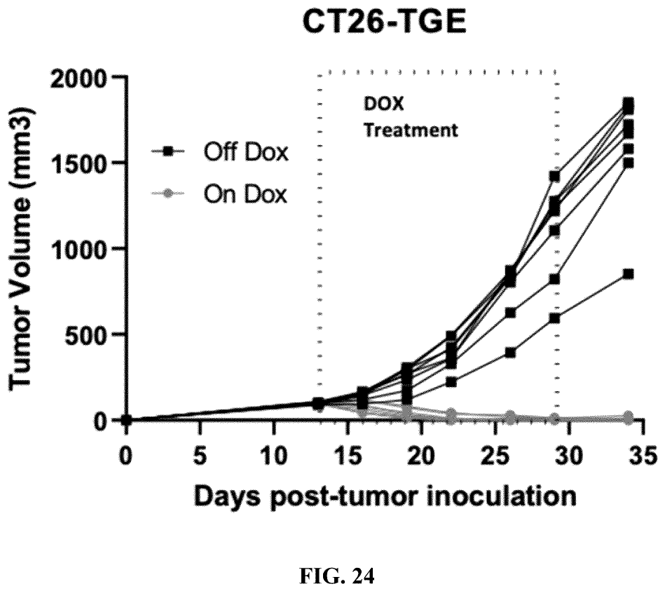

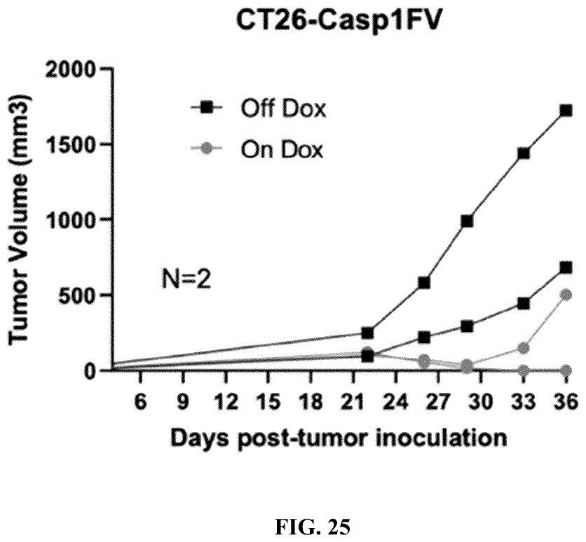

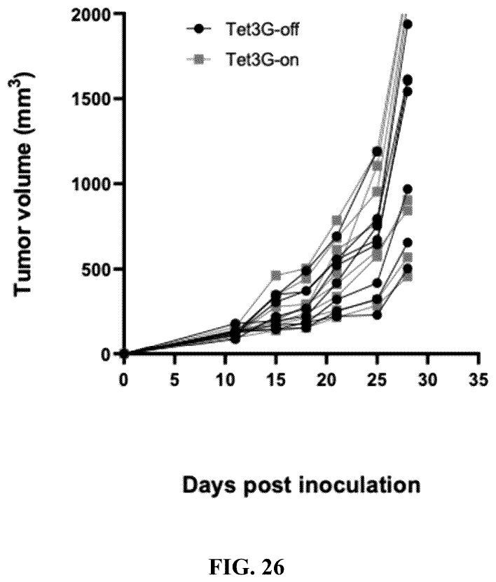

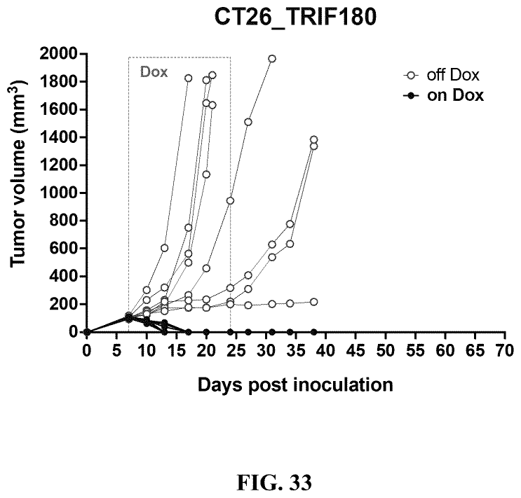

As shown in FIGS. 24 and 33, respectively, expression of TRIF+GSDME or TRIF_180 in colorectal carcinoma tumors resulted in complete tumor regression and sustained clearance. As shown in FIG. 23, expression of TRIF+Npro in colorectal carcinoma tumors initially resulted in tumor regression, followed by regrowth of the tumor several days after administration of doxycycline was terminated. As shown in FIG. 25, expression of CASP1FV in colorectal carcinoma tumors reduced tumor growth. As shown in FIG. 26, the empty Tet3G vector negative control did not affect tumor growth.

### Conclusion

These results demonstrate that expression of TRIF+GSDME or TRIF_180 in tumor cells results in sustained clearance of tumors in vivo, expression of TRIF+Npro results in tumor regression followed by tumor regrowth after termination of doxycycline administration, and that expression of the caspase 1 variant CASP1FV in tumor cells reduces tumor growth.

### Example 14. Preparation of mRNAs Encoding hTRIF, hGSDME or mIL-12

Each of the mRNAs prepared encoded a single polypeptide, i.e., human TRIF (hTRIF), human GSDME (hGSDME) or mouse IL-12 (mIL-12). Mouse IL-12 was used in these experiments because human IL-12 has minimal cross functional activity in mice. Despite limited cross reactivity, human and murine IL-12 play a highly homologous role in their respective immune responses. The mIL-12 mRNA contained a region encoding the IL-12 p40 subunit, a region encoding the IL-12 p35 subunit, and a region encoding a flexible linker (GGGGSGGGGSGGGGS; SEQ ID NO: 59) between the two IL-12 subunit encoding regions. The mRNAs encoding mIL-12, hTRIF, or hGSDME were in vitro transcribed in an enzymatic reaction containing an RNA polymerase, dNTPs, inorganic phosphatase, RNase inhibitor, and a plasmid DNA template. The plasmid DNA template, which contained the mRNA sequence cassette including an RNA polymerase promoter and a polyA tract of 120 As, was linearized with a restriction enzyme which cut into the polyA tract. The in vitro transcription reaction was stopped with the addition of DNase, and the mRNA was purified from the reaction mixture. In a subsequent reaction, a 5′ Cap1 structure was added to the mRNA using a capping enzyme and methyltransferase (the methyl donor was SAM-TOS). This final mRNA was purified from the reaction mixture. The mRNAs were formulated into LNPs before administration.

### Example 15. Evaluation of Intratumoral Injection of Combinations of mRNAs Encoding hTRIF, hGSDME or mIL-12 in a Mouse Model of Melanoma

### Methods

Female C57BL/6 mice were implanted with B16F10 murine melanoma cells in the rear-right hind flank and tumors were allowed to grow to a size of ˜100 mm³. At 100 mm³, mice were randomized, and mRNA and control treatments were administered intratumorally at 24 hour intervals on Day 0, 1, 2 and 3. Each mRNA encoded a single polypeptide (i.e., hTRIF, hGSDME or mIL-12), and was prepared as described above in Example 14. Each mouse received a total of 5 μg of LNP-formulated mIL-12 mRNA, either alone or in combination with LNP-formulated hTRIF and LNP-formulated hGSDME (7.5 μg+7.5 μg), or 20 μg of firefly luciferase (as a negative control) per injection. Four injections were given over 4 days, i.e., one injection per day. For the combination of mRNAs encoding hTRIF, mRNAs encoding hGSDME, and mRNAs encoding mIL-12, the mRNAs were administered at a ratio of 1:1:1 (i.e., hTRIF:hGSDME:mIL-12). Responses to the mRNAs were compared with treatment using a monoclonal antibody to mouse PD-1 (CD279, Clone RMP1-14, BioXCell, Catalogue No. BP0146), delivered intraperitoneally twice per week (b.i.w.) at a dose of 200 μg/mouse.

### Results

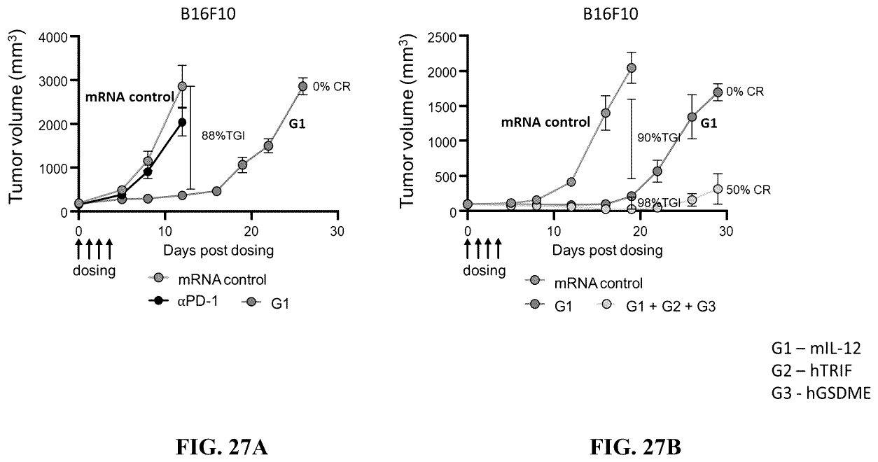

As shown in FIG. 27A, intratumoral injection of mRNA encoding mIL-12 reduced tumor growth relative to the firefly luciferase negative controls, with the greatest tumor growth inhibition observed between days 10 and 20, reaching 88-90% relative tumor growth inhibition to control. Intratumoral injection of mRNA encoding mIL-12 also reduced tumor growth relative to anti-PD-1 treatment. See FIG. 27A. The addition of mRNA encoding hTRIF and mRNA encoding hGSDME to mRNA encoding IL-12 greatly decreased tumor growth in comparison to IL-12 alone. See FIG. 27B. For example, at day 19, the delivery of mIL-12 mRNA alone induced 90% tumor growth inhibition (TGI), while the delivery of mIL-12 mRNA+hTRIF mRNA+hGSDME mRNA resulted in 98% TGI, relative to the luciferase mRNA negative control alone. On day 29, average tumor volume was approximately 250 mm³ for the mIL-12+hTRIF+hGSDME mRNA combination group, compared to approximately 1750 mm³ for the mIL-12 mRNA alone. See FIG. 27B. In addition, in the mIL-12+hTRIF+hGSDME mRNA combination group, 50% of mice showed complete tumor regression (CR), compared to 0% complete regression in the IL-12 alone or control groups.

### Conclusion

These results demonstrate that treatment of tumors with mRNAs encoding TRIF and GSDME polypeptides can effectively augment the efficacy of IL-12 in reducing tumor growth in vivo.

### Example 16. Effect of the Combination of Polynucleotides Encoding hTRIF and hGSDME on Murine Colorectal Carcinoma Cell Death

Methods: Murine colorectal carcinoma (CT-26) cells are transduced to stably express hTRIF+hGSDME under a doxycycline-inducible system. These cells are either left untreated (negative control) or cultured in the presence of 1 ug/mL doxycycline for 24 hrs. At 24 hrs, cell viability is measured using a CellTiter-Glo™ Luminescent assay. All samples are cultured in a BioTek Citation 5 Cell Imaging Multimode Reader with SYTOX Green and Hoechst stains to visualize dead and live cells, respectively. Images are captured every two hours for 24 hours.

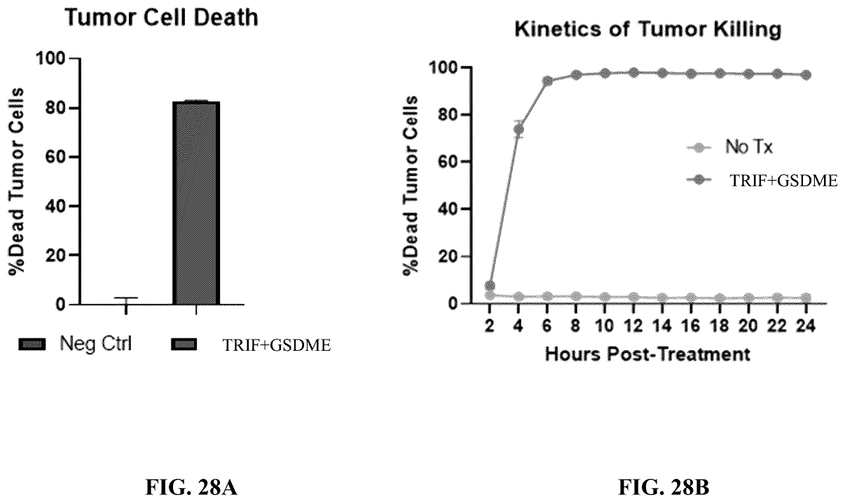

Results: Doxycycline-induced expression of hTRIF+hGSDME drives cell death in 80% of total CT-26 cells in culture after 24 hrs of doxycycline treatment. See FIG. 28A. Negligible cell death was observed in untreated cells during the same culture period. Doxycycline-induced expression of hTRIF+hGSDME causes cell death in ˜75% of the treated population by four hours of culture. By six hours of culture, doxycycline-treated cells were ˜95% dead. This remains unchanged for the remainder of the culture period. Untreated cells remain >90% viable during the full 24 hr time course.

Conclusion: These results indicate that activation of hTRIF+hGSDME expression results in cell death as early as 2 hours post-activation.

### Example 17. Effect of Cell Turnover Factors (CTFs) from Human Melanoma Cells Expressing hTRIF and hGSDME on CD80, CD40 and CD86 Expression in Monocyte-Derived Dendritic Cells (MoDCs)

### Methods

A375 human melanoma cells are transduced to stably express human htBid (negative control) or hTRIF+hGSDME under a doxycycline-inducible system. Cells are cultured in the presence of lug/mL doxycycline for 24 hrs. At 24 hrs, culture supernatants containing cell turnover factors (CTFs) are harvested and frozen until downstream use. Separately, human CD14+ monocytes are isolated from fresh blood samples and differentiated into immature dendritic cells using cytokines GM-CSF and IL-4 for 5 days. These immature monocyte-derived dendritic cells (MoDCs) are then cultured in a 1:1 mix of their normal growth medium and A375-derived CTFs for 24 hrs. Untreated MoDCs (data not shown) and LPS-treated (pos ctrl) MoDCs are also included for evaluation. After 24 hrs of culture, MoDCs are harvested and stained for surface activation markers. Cells are then processed on a flow cytometer to measure expression levels of these markers. The expression level reported is representative of the geometric mean fluorescent intensity measured on live, single cells.

### Results

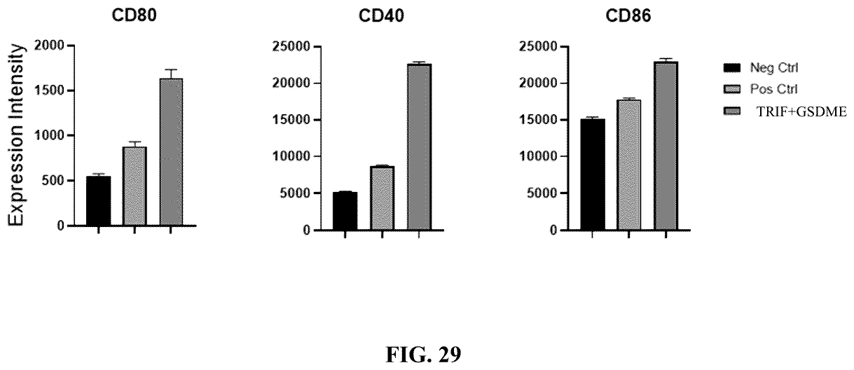

Untreated MoDCs performed similarly to MoDCs treated with CTF from the negative control A375 cells (data not shown). Relative to these baseline conditions, consistent trends were observed for each surface activation marker evaluated: CD80, CD40, and CD86. All markers were modestly upregulated with the positive control (LPS) treatment, which resulted in a 1.6-fold, 1.7-fold, and 1.2-fold increase in CD80, CD40, and CD86 expression levels, respectively. See FIG. 29. MoDCs exposed to CTFs derived from A375 cells expressing hTRIF+hGSDME showed even greater upregulation of these activation markers: 3.0-, 4.3-, and 1.5-fold increases for CD80, CD40, and CD86, respectively.

Conclusion: These results indicate that cell turnover factors secreted after activation of TRIF and GSDME expression can upregulate the expression of surface markers associated with antigen presentation on adjacent dendritic cells.

### Example 18. Expression of hTRIF and hGSDME in Human Colorectal Cancer Cells Upregulates MHC-1 Expression

Methods: Human colorectal cancer cells (HT29) are plated into 96-well plates and transduced with Adenovirus serotype 5 (Ad5, Neg Ctrl) or Ad5 encoding hTRIF and hGSDME at MOI 30. Virus containing media are removed 6 hours post transduction. HT29 cells are incubated with fresh media for 18 hours. Half of the media are removed before the addition of the same volume of fresh media containing immature monocyte derived dendritic cells (mo-DC) from healthy donors. HLA-ABC expression on live HT29 cells is analyzed 24 hours post mo-DC addition by flow cytometry.

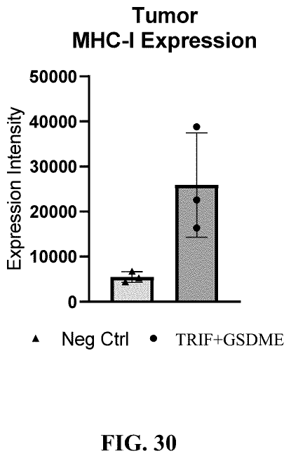

Results: As shown in FIG. 30, viral delivery of hTRIF+hGSDME polynucleotides in human tumor cells upregulates MHC-I expression on these tumor cells upon co-culture with human primary dendritic cells.

Conclusions: Expression of TRIF and GSDME increases the surface expression of MHC I, which is critical to overcome MHC downregulation, which is a major tumor evasion mechanism.

### Example 19. Effects of Expression of hTRIF and hGSDME Expression in Human Colorectal Cancer Cells Upregulates MHC-1 Expression

Methods: Female Balb/c mice are implanted with CT26 murine colon cancer cells engineered to express Tet3G transactivator alone (negative control) or hTRIF+hGSDME along with Tet3G. Tumors are allowed to grow to a size of ˜100 mm³ and switched to chow containing doxycycline (Dox) to induce hTRIF+hGSDME expression. Tumors are harvested 24 hours or 72 hours post Dox induction, processed into single cell suspension, and analyzed for Nkp46 expression within the CD45+%, CD206+% within CD11b+F4/80+(M2 macrophage %) and CD69 expression levels in CD45+CD3+CD4+CD4 T cells and CD45+CD3+CD8+CD8 T cells, respectively, by flow cytometry.

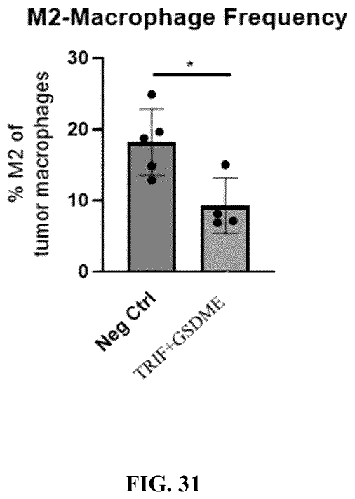

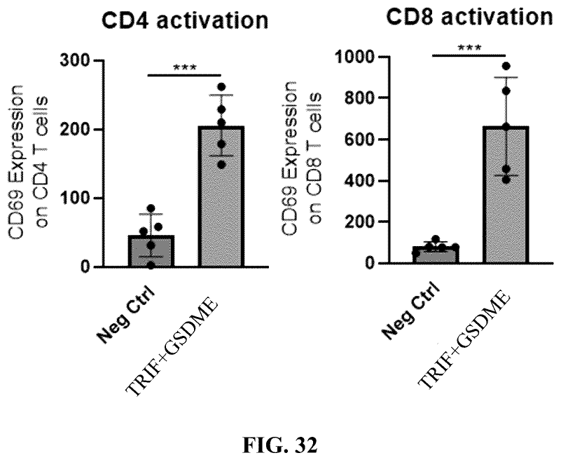

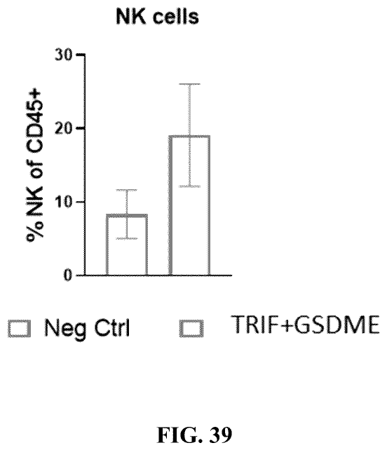

Results: Doxycycline-induced expression of hTRIF+hGSDME reduces the frequency of suppressive M2 macrophages (FIG. 31), while increasing the recruitment of NK cells (FIG. 39), and the activation of both CD4 and CD8 T cells within the tumor microenvironment (FIG. 32).

Conclusions: Expression of the combination of TRIF and GSDME results in pro-immune changes within the tumor microenvironment, whereby we see a reduction in suppressive M2-macrophages and an increase in NK cells, and an increase in the activation of CD4 and CD8 T cells.

### Example 20. Expression of Thanotransmission Polypeptides TRIF and GSDME Drives Activation of Both Antigen Specific and Non-Specific CD8⁺ T Cells within CT26 Tumors and are Required for Efficacy

### Methods

A series of murine colorectal carcinoma CT26 tumors were generated that expressed TRIF and GSDME under a doxycycline (Dox) inducible promoter. As a negative control, CT26 cells were also transduced with empty Tet3G vector. This negative control allowed for determination of any Dox induced anti-tumor effects. Six-week-old female BALB/c mice were inoculated with tumor cells contain dox-inducible thanotransmission polypeptides in the right flank. The Tet3G negative control tumors were inoculated at a concentration of 1e⁵ cells/100 μl, while the other treatment groups were inoculated at a concentration of 5e⁵ cells/100 uL. Mice were randomized when tumors reached 100 mm³ and divided equally into two groups, with one group given normal mouse chow, and the other normal mouse chow containing Dox.

At Day 3 following doxycycline, tumors were collected and subjected to digestion using a tumor dissociation kit (Miltenyi Biotech: 130-096-730) and a gentleMACS Octo Dissociator. Following digestion, cells were subjected to red blood cell lysis and counted. Cells were first FC blocked using a CD16/32 antibody, followed by staining with a live/dead exclusions stain and surface antibodies to CD45, TCRb, CD8, CD69, as well as a tetramer specific to the gp70 surface antigen. CD8 T cells were defined as CD45+TCRb+CD8+, gp70+ antigen specific T cells were defined as CD45+TCRb+CD8+gp70-tet+, activation status was determined by the surface expression of CD69.

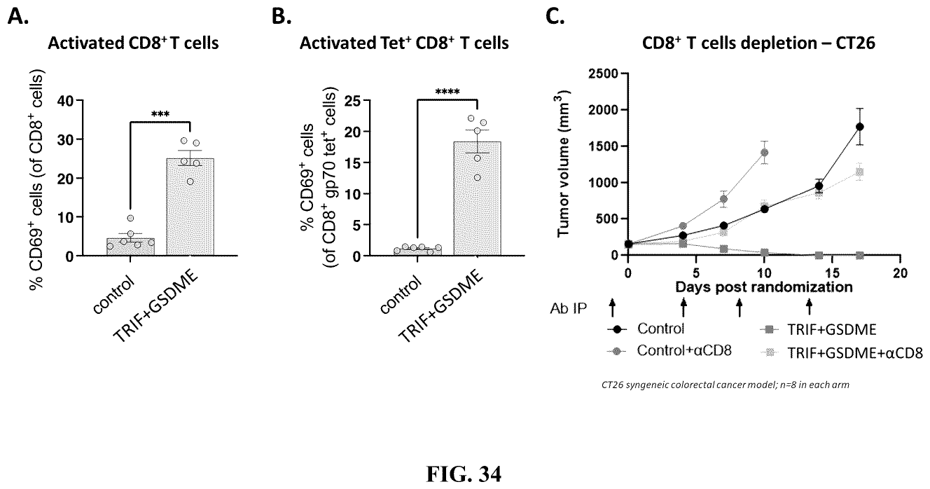

Results: Expression of thanotransmission polypeptides, TRIF+GSDME, resulted in a significant upregulation in activation markers on both total CD8 T cells, as well as antigen specific (gp70-tet+) T cells. See FIG. 34. The presence of CD8 T cells is essential to the efficacy of TRIF+GSDME in a CT26 tumor model, as the depletion of CD8 cells eliminates the efficacy.

Conclusions: TRIF+GSDME is a potent activator of CD8 T cells that when expressed in CT26 tumors are capable of generating robust anti-tumoral immunity.

### Example 21. Expression of Thanotransmission Polypeptides TRIF and GSDME Drives Significant Abscopal Effect in Distal Tumors of Mice Implanted with CT26 Tumors

### Methods

A series of murine colorectal carcinoma CT26 tumors were generated that expressed TRIF and GSDME under a doxycycline (Dox) inducible promoter. As a negative control, CT26 cells were also transduced with empty Tet3G vector. This negative control allowed for determination of any Dox induced anti-tumor effects. Six-week-old female BALB/c mice were initially inoculated with tumor cells contain dox-inducible thanotransmission polypeptides in the right flank. The Tet3G negative control tumors were inoculated at a concentration of 1e⁵ cells/100 μl, while the other treatment groups were inoculated at a concentration of 5e⁵ cells/100 uL. Four days after the right flank inoculation, 5e⁴ wild type CT26 cells were inoculated in the left flank. Mice were randomized when tumors reached 100 mm³ in the right flank and 40 mm³ in the left flank and divided equally into two groups, with one group given normal mouse chow, and the other normal mouse chow containing Dox. Further, half of each group were given i.p. 200 μg of an anti-PD1 blocking antibody at day 16 and every three days to a total of 3 doses (BioXcell, Catalogue No. BP0273) and isotype control.

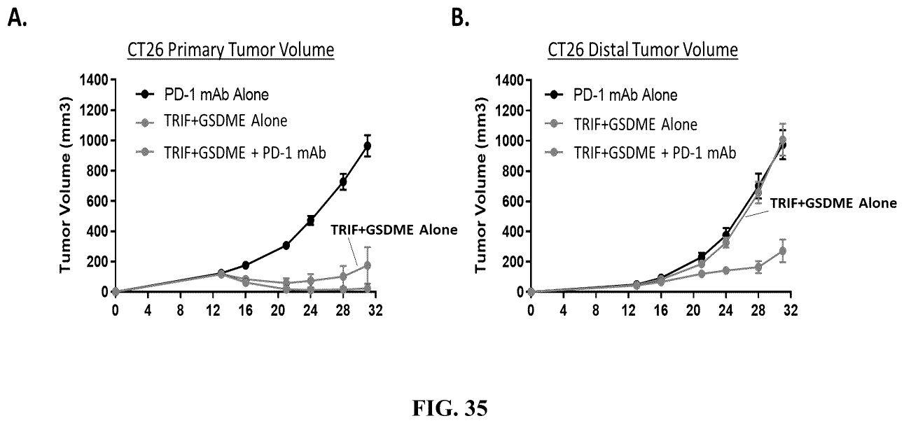

Results: The addition of anti-PD1 antibody resulted in the activation of a significant abscopal effect, as demonstrated by the shrinkage of the untreated bi-flank tumor. See FIG. 35. While anti-PD1 antibody is not required for efficacy in the primary treated tumor, anti-PD1 antibody is essential to systemic immune mediated regression of the bi-flank tumor, which shows a tumor growth inhibition (TGI) of ˜80%, compared with 0% in matched isotype treated mice.

Conclusions: Expression of thanotransmission polypeptides TRIF+GSDME can synergize with anti-PD1 antibody delivering robust systemic anti-tumoral control, demonstrating the ability to target metastatic lesions following local administration

### Example 22. Delivery of LNP-mRNAs Encoding Thanotransmission Polypeptides TRIF and GSDME Along with IL-12 Drives Significant Abscopal Effect in Distal Tumors of Mice Implanted with B16F10 Tumors

### Methods

Female C57BL/6 mice were implanted with B16F10 murine melanoma cells in the rear-right hind flank and two days later, 5E4 B16F10 murine melanoma cells were implanted in the rear left hind flanks and tumors were allowed to grow to a size of ˜80 mm³ on the right and ˜40 mm³ on the left, at which point mice were randomized, and mRNA and control treatments were administered intratumorally at 24 hour intervals on Day 0, 1, 2 and 3. Each mRNA encoded a single thanotransmission polypeptide (i.e., TRIF or GSDME) or IL-12 and were prepared as described above in Example 14. For each of the four injections, each mouse received a total of 20 μg of LNP-formulated mRNA encoding, TRIF+GSDME+IL-12 (ratio of 1:1:0.66) or firefly luciferase (as a negative control).

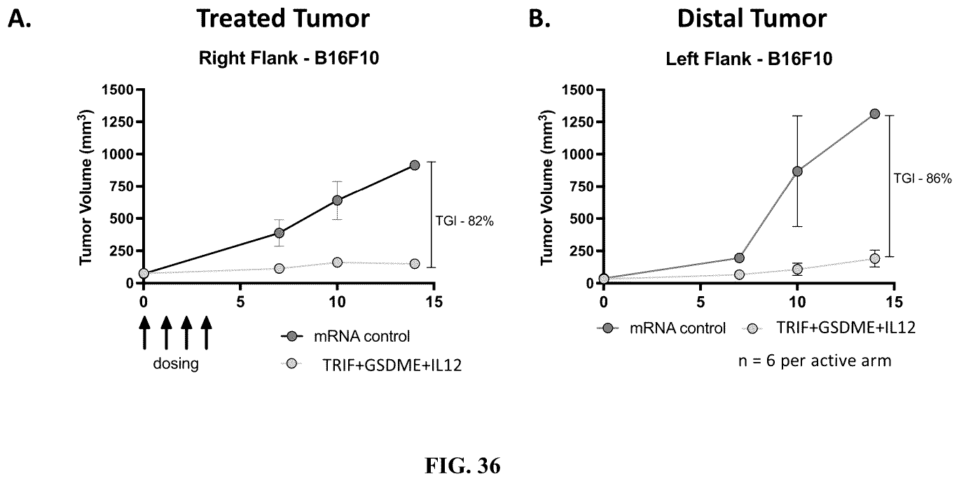

Results: LNP-mRNA delivery of TRIF+GSDME+IL-12 resulted in a potent abscopal effect, demonstrating ˜86% TGI in the distal untreated tumor. See FIG. 36.

Conclusions: TRIF+GSDME+IL-12 provides control over the distal tumor, demonstrating the ability to target metastatic lesions following local administration.

### Example 23. In Vitro Delivery of mRNAs Encoding Thanotransmission Polypeptides TRIF and GSDME Along with IL-12 Drives Immunogenic Cell Death

Methods:

Each mRNA encoded a single polypeptide, i.e., Luc, TRIF, IL-12, an apoptosis inducer, or GSDME. All of the mRNAs used in these experiments contained (from 5′ to 3′) a 5′ cap structure, a 5′ UTR, a thanotransmission polypeptide coding region (i.e., TRIF or GSDME) or other mRNA (Luc, IL-12, tBid), a 3′ UTR, and a polyA region. The mRNAs encoding genes of interest were in vitro transcribed in an enzymatic reaction containing an RNA polymerase, dNTPs, inorganic phosphatase, RNase inhibitor, and a plasmid DNA template. The plasmid DNA template, which contained the mRNA sequence cassette including a RNA polymerase promoter and a polyA tract of 120 As, was linearized with a restriction enzyme which cut into the polyA tract. The in vitro transcription reaction was stopped with the addition of DNAse, and the mRNA was purified from the reaction mixture. In a subsequent reaction, a 5′ Cap1 structure was added to the mRNA using a capping enzyme and methyltransferase (the methyl donor was SAM-TOS). This final mRNA was purified from the reaction mixture.

B16F10 murine melanoma cells were plated at the manufactures recommended density for transfection using the MessengerMax in vitro transfection kit. The mRNAs encoding a single thanotransmission polypeptide were combined to test combinations of the thanotransmission polypeptides. The treatment groups tested are shown below.
1. firefly luciferase
2. IL-12
3. TRIF, GSDME, IL-12
4. Apoptosis inducer
5. Apoptosis inducer+IL-12

The total amount of transfected mRNA was normalized using firefly luciferase. Cell viability was determined by CellTiter-Glo® Luminescent Cell Viability Assay. Membrane permeability was determined as % Sytox⁺ using an Incucyte over 24 h. High mobility group box 1 (HMGB1) was determined using a specific ELISA according to the manufacturers protocol. Differential production of pro-inflammatory cytokines and chemokines were determined using a mouse antiviral Biolegend LegendPlex kit and expressed as Log 2 fold change from the average expression of the mRNA control. Three replicates for each condition were included.

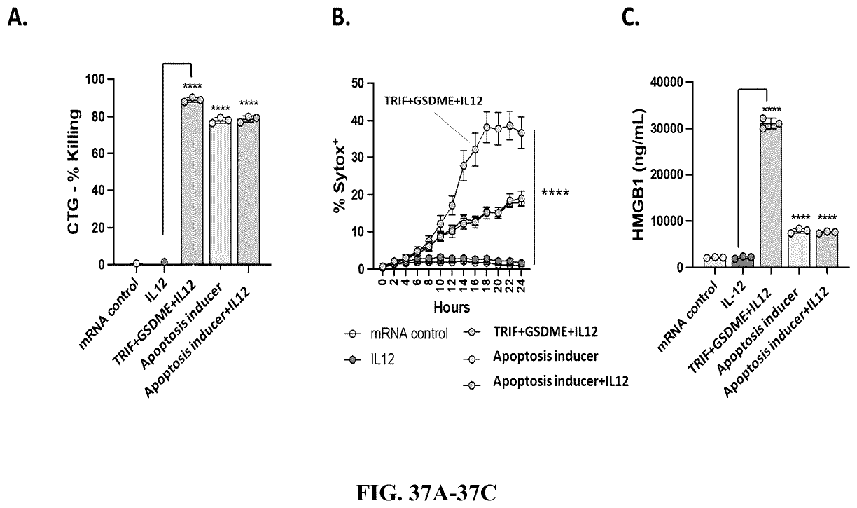

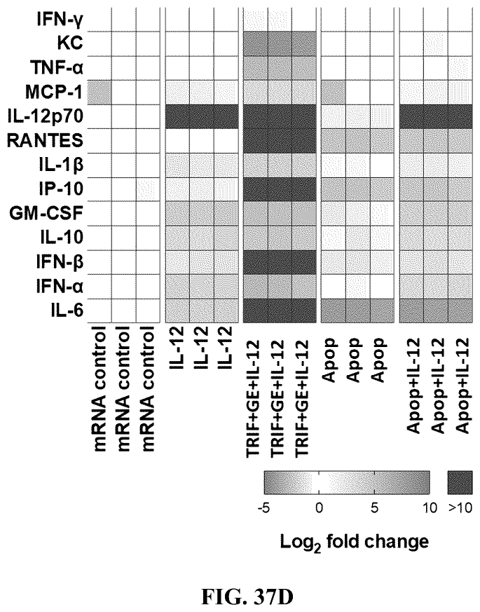

Results: The co-transfection of TRIF+GSDME+IL-12 induces a cell death that is similar in magnitude to a potent and direct inducer of apoptosis. See FIG. 37. However, unlike the apoptosis inducer, TRIF+GSDME+IL-12 drives a unique form of immunogenic cell death, characterized by the enhanced membrane permeability and release of HMGB1. Likewise, expression of IL-12 has no effect on any parameters of cell death. Moreover, TRIF+GSDME+IL-12 drives a highly unique cytokine profile that is distinct from IL-12 alone.

Conclusions: TRIF+GSDME+IL-12 drives a distinct form of immunogenic cell death that likely contributes to enhanced in vivo efficacy.

### Example 24. Delivery of LNP-mRNAs Encoding Thanotransmission Polypeptides TRIF and GSDME Along with IL-12 Drives Significant Early Tumor Shrinkage and Activation of Antigen Specific CD8+ T Cells

### Methods:

Female C57BL/6 mice were implanted with 1E5 B16F10 murine melanoma cells in the rear-right hind flank and two days later, 5E4 B16F10 murine melanoma cells were implanted in the rear left hind flanks and tumors were allowed to grow to a size of ˜80 mm³ on the right and ˜40 mm³ on the left, at which point mice were randomized, and mRNA and control treatments were administered intratumorally at 24 hour intervals on Day 0, 1, 2 and 3. Each mRNA encoded a single thanotransmission polypeptide (i.e., TRIF or GSDME) or IL-12 and were prepared as described above. For each of the four injections, each mouse received a total of 20 μg of LNP-formulated mRNA encoding, TRIF+GSDME+IL-12 (ratio of 1:1:0.66), IL-12+Luc (0.33:1) or firefly luciferase (as a negative control). At Day 12 following initial dosing, tumors were collected and subjected to digestion using a tumor dissociation kit (Miltenyi Biotech: 130-096-730) and a gentleMACS Octo Dissociator. Following digestion, cells were subjected to red blood cell lysis and were then counted. The cells were initially FC blocked using a CD16/32 antibody, followed by staining with a live/dead exclusion stain and surface antibodies to CD45, TCRb, CD8, CD69, as well as a tetramer specific to the gp100 surface antigen. CD8 T cells were classified as CD45+TCRb+CD8+, and gp100+ antigen-specific T cells were identified as CD45+TCRb+CD8+gp100-tet+. The activation status was gauged by the surface expression of CD69.

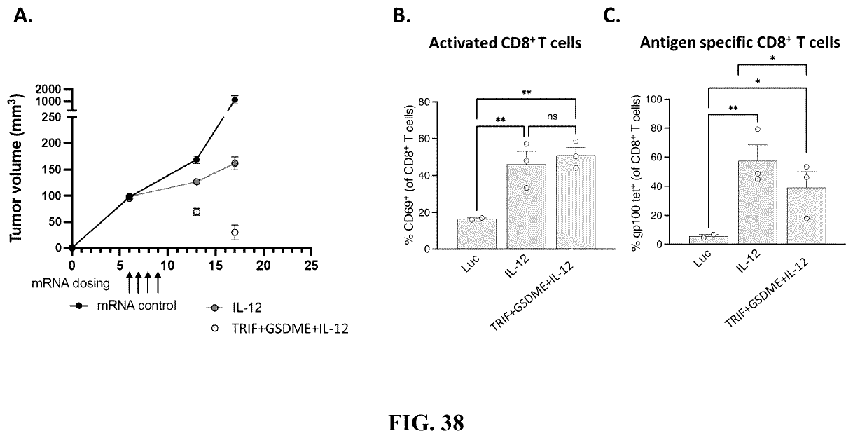

Results: The delivery of TRIF+GSDME+IL-12 resulted in significant early regression in B16F10, while IL-12 delayed tumor growth, but resulted in no significant regression. See FIG. 38. Despite inducing similar levels of antigen specific T cells, TRIF+GSDME+IL-12 exhibited significantly enhanced efficacy in reducing tumor growth when compared with IL-12 alone.

Conclusions: The unique combinational effects of TRIF+GSDME+IL-12 resulted in significantly decreased tumor growth in the B16F10 tumor model.

DESCRIPTION OF SEQUENCES

| SEQ | |
|---|---|
| ID NO: | Description |
| 1 | Human wildtype TRIF nucleic acid sequence |
| 2 | Human wildtype TRIF amino acid sequence ((UniProtKB - |
| | Q8IUC6) |
| 3 | TRIF_mutRHIM nucleic acid sequence |
| | Mutation of the human TRIF RHIM tetrad into AAAA |
| | (688-691 - QLGL → AAAA) |
| 4 | TRIF_mutRHIM amino acid sequence |
| 5 | TRIF_Trunc nucleic acid sequence |
| | Truncation of the C-ter fragment (541-712) of human TRIF |
| | containing the RHIM domain |
| 6 | TRIF_Trunc amino acid sequence |
| 7 | TRIF_PhosphoM nucleic acid sequence |
| | Mutations of human TRIF for TBK1 phosphorylation sites |
| | (S210A, S212A, T214A) |
| 8 | TRIF_PhosphoM amino acid sequence |
| 9 | TRIF_P434H nucleic acid sequence |
| | Mutation for dimerization site P434 in the TIR domain |
| | of human TRIF |
| 10 | TRIF_P434H amino acid sequence |
| 11 | miniTRIF nucleic acid sequence |
| | N-terminal deletion (1-311) of human TRIF |
| 12 | miniTRIF amino acid sequence |
| 13 | TRIF_d1-180 nucleic acid sequence |
| | N-terminal deletion (1-180) of human TRIF |
| 14 | TRIF_d1-180 amino acid sequence |
| 15 | TIR domain nucleic acid sequence |
| | Fragment 387-544 of human TRIF |
| 16 | TIR domain amino acid sequence |
| 17 | TRIS nucleic acid sequence |
| | Deletion of N-terminal fragment 1-180 and fragment |
| | 217-658 of human TRIF |
| 18 | TRIS amino acid sequence |
| 19 | TRIR nucleic acid sequence |
| | Deletion of N-terminal fragment 1-180, fragment 217-386 |
| | and fragment 546-712 of human TRIF |
| 20 | TRIR amino acid sequence |
| 21 | TRIR3 nucleic acid sequence |
| | TRIR followed by a flexible linker (GPGGSSGSS; |
| | SEQ ID NO: 25) and hRIPK3 (UniProtKB - Q9Y572 |
| | (RIPK3_HUMAN)) |
| 22 | TRIR3 amino acid sequence |
| 23 | FLAG-tag nucleic acid sequence |
| 24 | FLAG-tag amino acid sequence |
| 25 | Flexible Linker amino acid sequence (GPGGSSGSS) |
| 26 | T2A peptide amino acid sequence |
| 27 | P2A peptide amino acid sequence |
| 28 | E2A peptide amino acid sequence |
| 29 | F2A peptide amino acid sequence |
| 30 | Human wildtype RIPK3 amino acid sequence |
| 31 | Human wildtype RIPK3 nucleic acid sequence |
| 32 | vICA amino acid sequence |
| 33 | 5′ UTR RNA sequence |
| 34 | 3′UTR RNA sequence, Variation 1 comprising RNA |
| | sequence encoding miR-122-5p binding site |
| 35 | 3′UTR RNA sequence, Variation 2 comprising RNA |
| | sequence encoding miR-122-5p binding site, and RNA |
| | sequence encoding miR-142-3p binding site |
| 36 | miR-122-5p binding site RNA sequence |
| 37 | miR-142-3p binding site RNA sequence |
| 38 | Poly A tail |
| 39 | Human TRIF DNA construct comprising 5′UTR, TRIF |
| | coding region, 3′UTR Variation 1, and polyA tail |
| 40 | Human GSDME DNA construct comprising 5′UTR, GSDME |
| | coding region, 3′UTR Variation 1, and polyA tail |
| 41 | Mouse RIPK3 DNA construct comprising 5′UTR, mRIPK3 |
| | coding region, 3′UTR Variation 1, and polyA tail |
| 42 | Human RIPK3 DNA construct comprising 5′UTR, hRIPK3 |
| | coding region, 3′UTR Variation 1, and poly A tail |
| 43 | vICA DNA construct comprising 5′UTR, vICA coding |
| | region, 3′UTR Variation 1, and poly A tail |
| 44 | Human wildtype Gasdermin E nucleic acid sequence |
| 45 | Viral wildtype Npro nucleic acid sequence |
| 46 | Viral wildtype A238L nucleic acid sequence |
| 47 | Viral wildtype vMLKL nucleic acid sequence |
| 48 | Viral vICA nucleic acid sequence |
| 49 | IKBa dominant negative variant (IKBa_DN) nucleic |
| | acid sequence |
| 50 | IKBa human wildtype amino acid sequence |
| 51 | Human wildtype Caspase 1 full-length nucleic acid sequence |
| 52 | Human wildtype Caspase 1 full-length amino acid sequence |
| 53 | Human Caspase 1 fragment amino acid sequence |
| 54 | Human Caspase 1 fragment with self-dimerizing domain added |
| | to C-terminus (CASP1FV) amino acid sequence |
| 55 | CASP1FV nucleic acid sequence |
| 56 | Mouse IL-12 p40 subunit nucleic acid sequence |
| 57 | Mouse IL-12 p35 subunit nucleic acid sequence |
| 58 | G4S₃ linker nucleic acid sequence |
| 59 | G4S₃ linker |
| 60 | Human IL12A (p35) amino acid sequence (GenBank: |
| | AF101062.1) |
| 61 | Human IL12A (p35) nucleic acid sequence |
| 62 | Human IL12B (p40) amino acid sequence (GenBank: |
| | AF180563.1) |
| 63 | Human IL12B (p40) nucleic acid sequence |
| 64 | mIL-12 mRNA construct from Example 15 |
| 65 | hTRIF mRNA construct from Example 15 |
| 66 | hGSDME mRNA construct from Example 15 |
| 67 | GGGGS linker |
| 68 | DNA sequence of alternative 3'UTR comprising |
| | miR-122-5p binding site and miR-142-3p binding site |

SEQUENCES OF THE DISCLOSURE
hTRIF_WT (UniProtKB-Q8IUC6 (TCAM1_HUMAN)) “TRIF_WT”
SEQ ID NO: 1
ATGGCTTGCACAGGACCTTCTCTGCCCAGCGCCTTTGATATCCTGGGAGCCGCTGGACAGGACAAGCTGCTGTAC
CTGAAGCACAAGCTGAAAACCCCTCGGCCTGGCTGCCAGGGACAAGATCTGCTGCATGCTATGGTGCTGCTGAAG
CTGGGCCAAGAGACAGAGGCCAGAATCAGCCTGGAAGCCCTGAAGGCTGATGCCGTGGCTAGACTGGTTGCCAGA
CAATGGGCTGGCGTGGACAGCACAGAGGACCCTGAAGAACCTCCTGACGTGTCCTGGGCCGTCGCCAGACTGTAT
CATCTGCTGGCCGAAGAGAAGCTGTGCCCCGCCTCTCTGAGAGATGTGGCCTATCAAGAAGCCGTGCGGACCCTG
AGCAGCAGGGATGATCATAGACTGGGCGAGCTGCAGGACGAGGCCCGGAATAGATGTGGCTGGGATATTGCTGGC
GACCCCGGCAGCATTAGAACCCTGCAGTCTAACCTGGGCTGCCTGCCTCCATCTTCTGCCCTGCCATCTGGCACA
AGAAGCCTGCCTAGACCTATCGACGGCGTGTCCGATTGGAGCCAGGGCTGTTCTCTGAGAAGCACAGGATCTCCA
GCCAGCCTGGCCAGCAACCTGGAAATCAGCCAGTCTCCTACAATGCCCTTCCTGAGCCTGCACAGAAGCCCTCAC
GGACCTAGCAAGCTGTGCGACGATCCTCAGGCTTCTCTGGTGCCTGAACCTGTTCCTGGCGGCTGCCAAGAGCCT
GAAGAGATGTCTTGGCCTCCTAGCGGCGAGATCGCCTCTCCACCTGAACTGCCATCTAGCCCTCCACCAGGACTG
CCTGAAGTGGCCCCTGATGCCACTTCTACAGGCCTGCCTGATACACCCGCCGCTCCAGAGACAAGCACAAACTAC
CCTGTGGAATGCACCGAGGGCTCTGCCGGACCTCAATCTCTGCCTCTGCCTATCCTGGAACCTGTGAAGAACCCT
TGCAGCGTGAAGGATCAGACCCCTCTGCAGCTGAGCGTGGAAGATACCACCTCTCCTAACACCAAGCCTTGTCCT
CCAACACCTACCACACCTGAGACAAGCCCACCTCCTCCGCCTCCACCACCAAGCTCTACACCTTGTAGCGCCCAC
CTGACACCAAGCAGCCTGTTTCCAAGCTCTCTGGAAAGCAGCAGCGAGCAGAAATTCTACAACTTCGTGATCCTG
CACGCCAGAGCCGACGAGCACATTGCCCTGAGAGTGCGCGAAAAGCTGGAAGCTCTGGGAGTGCCTGATGGCGCC
ACCTTCTGCGAGGATTTTCAGGTTCCCGGAAGAGGCGAGCTGAGCTGTCTGCAGGATGCCATCGATCACAGCGCC
TTCATCATTCTGCTGCTGACCAGCAACTTCGACTGCCGGCTGTCTCTGCACCAAGTGAACCAGGCCATGATGAGC
AACCTGACCAGACAGGGCAGCCCCGATTGCGTGATCCCATTCCTGCCACTGGAAAGCTCCCCAGCACAGCTGTCT
AGCGATACTGCCTCTCTGCTGTCTGGACTCGTGCGGCTGGATGAGCACAGCCAGATCTTCGCCAGAAAGGTGGCC
AACACCTTCAAGCCCCATCGGCTGCAGGCCAGAAAAGCCATGTGGCGGAAAGAGCAGGACACACGGGCACTGAGA
GAGCAGTCTCAGCACCTGGATGGCGAGAGAATGCAGGCCGCTGCTCTGAATGCCGCCTACAGCGCTTACCTGCAG
AGCTACCTGAGCTATCAGGCCCAGATGGAACAGCTGCAGGTCGCCTTTGGCAGCCACATGTCCTTTGGAACAGGC
GCCCCTTACGGCGCCAGAATGCCTTTTGGTGGACAGGTGCCACTGGGAGCCCCTCCACCTTTTCCAACATGGCCA
GGATGTCCCCAGCCTCCTCCACTGCATGCTTGGCAAGCTGGAACACCTCCGCCACCATCTCCACAGCCAGCTGCC
TTTCCACAGTCTCTCCCATTTCCACAGAGCCCCGCCTTTCCAACAGCTAGCCCTGCTCCTCCACAAAGCCCTGGA
CTGCAGCCCCTGATCATTCACCACGCACAGATGGTGCAGCTGGGACTGAACAATCACATGTGGAACCAGAGAGGC
TCTCAGGCCCCTGAGGACAAGACACAAGAGGCCGAAtga
SEQ ID NO: 2
MACTGPSLPSAFDILGAAGQDKLLYLKHKLKTPRPGCQGQDLLHAMVLLKLGQETEARISLEALKADAVARLVAR
QWAGVDSTEDPEEPPDVSWAVARLYHLLAEEKLCPASLRDVAYQEAVRTLSSRDDHRLGELQDEARNRCGWDIAG
DPGSIRTLQSNLGCLPPSSALPSGTRSLPRPIDGVSDWSQGCSLRSTGSPASLASNLEISQSPTMPFLSLHRSPH
GPSKLCDDPQASLVPEPVPGGCQEPEEMSWPPSGEIASPPELPSSPPPGLPEVAPDATSTGLPDTPAAPETSTNY
PVECTEGSAGPQSLPLPILEPVKNPCSVKDQTPLQLSVEDTTSPNTKPCPPTPTTPETSPPPPPPPPSSTPCSAH
LTPSSLFPSSLESSSEQKFYNFVILHARADEHIALRVREKLEALGVPDGATFCEDFQVPGRGELSCLQDAIDHSA
FIILLLTSNFDCRLSLHQVNQAMMSNLTRQGSPDCVIPFLPLESSPAQLSSDTASLLSGLVRLDEHSQIFARKVA
NTFKPHRLQARKAMWRKEQDTRALREQSQHLDGERMQAAALNAAYSAYLQSYLSYQAQMEQLQVAFGSHMSFGTG
APYGARMPFGGQVPLGAPPPFPTWPGCPQPPPLHAWQAGTPPPPSPQPAAFPQSLPFPQSPAFPTASPAPPQSPG
LQPLIIHHAQMVQLGLNNHMWNQRGSQAPEDKTQEAE
hTRIF_mutRHIM “TRIF_mutRHIM”
Mutation of the RHIM tetrad into AAAA (688-691-QLGL → AAAA)
SEQ ID NO: 3
ATGGCTTGCACAGGACCTTCTCTGCCCAGCGCCTTTGATATCCTGGGAGCCGCTGGACAGGACAAGCTGCTGTAC
CTGAAGCACAAGCTGAAAACCCCTCGGCCTGGCTGCCAGGGACAAGATCTGCTGCATGCTATGGTGCTGCTGAAG
CTGGGCCAAGAGACAGAGGCCAGAATCAGCCTGGAAGCCCTGAAGGCTGATGCCGTGGCTAGACTGGTTGCCAGA
CAATGGGCTGGCGTGGACAGCACAGAGGACCCTGAAGAACCTCCTGACGTGTCCTGGGCCGTCGCCAGACTGTAT
CATCTGCTGGCCGAAGAGAAGCTGTGCCCCGCCTCTCTGAGAGATGTGGCCTATCAAGAAGCCGTGCGGACCCTG
AGCAGCAGGGATGATCATAGACTGGGCGAGCTGCAGGACGAGGCCCGGAATAGATGTGGCTGGGATATTGCTGGC
GACCCCGGCAGCATTAGAACCCTGCAGTCTAACCTGGGCTGCCTGCCTCCATCTTCTGCCCTGCCATCTGGCACA
AGAAGCCTGCCTAGACCTATCGACGGCGTGTCCGATTGGAGCCAGGGCTGTTCTCTGAGAAGCACAGGATCTCCA
GCCAGCCTGGCCAGCAACCTGGAAATCAGCCAGTCTCCTACAATGCCCTTCCTGAGCCTGCACAGAAGCCCTCAC
GGACCTAGCAAGCTGTGCGACGATCCTCAGGCTTCTCTGGTGCCTGAACCTGTTCCTGGCGGCTGCCAAGAGCCT
GAAGAGATGTCTTGGCCTCCTAGCGGCGAGATCGCCTCTCCACCTGAACTGCCATCTAGCCCTCCACCAGGACTG
CCTGAAGTGGCCCCTGATGCCACTTCTACAGGCCTGCCTGATACACCCGCCGCTCCAGAGACAAGCACAAACTAC
CCTGTGGAATGCACCGAGGGCTCTGCCGGACCTCAATCTCTGCCTCTGCCTATCCTGGAACCTGTGAAGAACCCT
TGCAGCGTGAAGGATCAGACCCCTCTGCAGCTGAGCGTGGAAGATACCACCTCTCCTAACACCAAGCCTTGTCCT
CCAACACCTACCACACCTGAGACAAGCCCACCTCCTCCGCCTCCACCACCAAGCTCTACACCTTGTAGCGCCCAC
CTGACACCAAGCAGCCTGTTTCCAAGCTCTCTGGAAAGCAGCAGCGAGCAGAAATTCTACAACTTCGTGATCCTG
CACGCCAGAGCCGACGAGCACATTGCCCTGAGAGTGCGCGAAAAGCTGGAAGCTCTGGGAGTGCCTGATGGCGCC
ACCTTCTGCGAGGATTTTCAGGTTCCCGGAAGAGGCGAGCTGAGCTGTCTGCAGGATGCCATCGATCACAGCGCC
TTCATCATTCTGCTGCTGACCAGCAACTTCGACTGCCGGCTGTCTCTGCACCAAGTGAACCAGGCCATGATGAGC
AACCTGACCAGACAGGGCAGCCCCGATTGCGTGATCCCATTCCTGCCACTGGAAAGCTCCCCAGCACAGCTGTCT
AGCGATACTGCCTCTCTGCTGTCTGGACTCGTGCGGCTGGATGAGCACAGCCAGATCTTCGCCAGAAAGGTGGCC
AACACCTTCAAGCCCCATCGGCTGCAGGCCAGAAAAGCCATGTGGCGGAAAGAGCAGGACACACGGGCACTGAGA
GAGCAGTCTCAGCACCTGGATGGCGAGAGAATGCAGGCCGCTGCTCTGAATGCCGCCTACAGCGCTTACCTGCAG
AGCTACCTGAGCTATCAGGCCCAGATGGAACAGCTGCAGGTCGCCTTTGGCAGCCACATGTCCTTTGGAACAGGC
GCCCCTTACGGCGCCAGAATGCCTTTTGGTGGACAGGTGCCACTGGGAGCCCCTCCACCTTTTCCAACATGGCCA
GGATGTCCCCAGCCTCCTCCACTGCATGCTTGGCAAGCTGGAACACCTCCGCCACCATCTCCACAGCCAGCTGCC
TTTCCACAGTCTCTCCCATTTCCACAGAGCCCCGCCTTTCCAACAGCTAGCCCTGCTCCTCCACAAAGCCCTGGA
CTGCAGCCCCTGATCATTCACCACGCACAGATGGTGgcagcggccgctAACAATCACATGTGGAACCAGAGAGGC
TCTCAGGCCCCTGAGGACAAGACACAAGAGGCCGAAtga
SEQ ID NO: 4
MACTGPSLPSAFDILGAAGQDKLLYLKHKLKTPRPGCQGQDLLHAMVLLKLGQETEARISLEALKADAVARLVAR
QWAGVDSTEDPEEPPDVSWAVARLYHLLAEEKLCPASLRDVAYQEAVRTLSSRDDHRLGELQDEARNRCGWDIAG
DPGSIRTLQSNLGCLPPSSALPSGTRSLPRPIDGVSDWSQGCSLRSTGSPASLASNLEISQSPTMPFLSLHRSPH
GPSKLCDDPQASLVPEPVPGGCQEPEEMSWPPSGEIASPPELPSSPPPGLPEVAPDATSTGLPDTPAAPETSTNY
PVECTEGSAGPQSLPLPILEPVKNPCSVKDQTPLQLSVEDTTSPNTKPCPPTPTTPETSPPPPPPPPSSTPCSAH
LTPSSLFPSSLESSSEQKFYNFVILHARADEHIALRVREKLEALGVPDGATFCEDFQVPGRGELSCLQDAIDHSA
FIILLLTSNFDCRLSLHQVNQAMMSNLTRQGSPDCVIPFLPLESSPAQLSSDTASLLSGLVRLDEHSQIFARKVA
NTFKPHRLQARKAMWRKEQDTRALREQSQHLDGERMQAAALNAAYSAYLQSYLSYQAQMEQLQVAFGSHMSFGTG
APYGARMPFGGQVPLGAPPPFPTWPGCPQPPPLHAWQAGTPPPPSPQPAAFPQSLPFPQSPAFPTASPAPPQSPG
LQPLIIHHAQMVAAAANNHMWNQRGSQAPEDKTQEAE
hTRIF_CterTrunc “TRIF_Trunc”
Truncation of the C-ter fragment (541-712) containing the RHIM domain
SEQ ID NO: 5
ATGGCTTGCACAGGACCTTCTCTGCCCAGCGCCTTTGATATCCTGGGAGCCGCTGGACAGGACAAGCTGCTGTAC
CTGAAGCACAAGCTGAAAACCCCTCGGCCTGGCTGCCAGGGACAAGATCTGCTGCATGCTATGGTGCTGCTGAAG
CTGGGCCAAGAGACAGAGGCCAGAATCAGCCTGGAAGCCCTGAAGGCTGATGCCGTGGCTAGACTGGTTGCCAGA
CAATGGGCTGGCGTGGACAGCACAGAGGACCCTGAAGAACCTCCTGACGTGTCCTGGGCCGTCGCCAGACTGTAT
CATCTGCTGGCCGAAGAGAAGCTGTGCCCCGCCTCTCTGAGAGATGTGGCCTATCAAGAAGCCGTGCGGACCCTG
AGCAGCAGGGATGATCATAGACTGGGCGAGCTGCAGGACGAGGCCCGGAATAGATGTGGCTGGGATATTGCTGGC
GACCCCGGCAGCATTAGAACCCTGCAGTCTAACCTGGGCTGCCTGCCTCCATCTTCTGCCCTGCCATCTGGCACA
AGAAGCCTGCCTAGACCTATCGACGGCGTGTCCGATTGGAGCCAGGGCTGTTCTCTGAGAAGCACAGGATCTCCA
GCCAGCCTGGCCAGCAACCTGGAAATCAGCCAGTCTCCTACAATGCCCTTCCTGAGCCTGCACAGAAGCCCTCAC
GGACCTAGCAAGCTGTGCGACGATCCTCAGGCTTCTCTGGTGCCTGAACCTGTTCCTGGCGGCTGCCAAGAGCCT
GAAGAGATGTCTTGGCCTCCTAGCGGCGAGATCGCCTCTCCACCTGAACTGCCATCTAGCCCTCCACCAGGACTG
CCTGAAGTGGCCCCTGATGCCACTTCTACAGGCCTGCCTGATACACCCGCCGCTCCAGAGACAAGCACAAACTAC
CCTGTGGAATGCACCGAGGGCTCTGCCGGACCTCAATCTCTGCCTCTGCCTATCCTGGAACCTGTGAAGAACCCT
TGCAGCGTGAAGGATCAGACCCCTCTGCAGCTGAGCGTGGAAGATACCACCTCTCCTAACACCAAGCCTTGTCCT
CCAACACCTACCACACCTGAGACAAGCCCACCTCCTCCGCCTCCACCACCAAGCTCTACACCTTGTAGCGCCCAC
CTGACACCAAGCAGCCTGTTTCCAAGCTCTCTGGAAAGCAGCAGCGAGCAGAAATTCTACAACTTCGTGATCCTG
CACGCCAGAGCCGACGAGCACATTGCCCTGAGAGTGCGCGAAAAGCTGGAAGCTCTGGGAGTGCCTGATGGCGCC
ACCTTCTGCGAGGATTTTCAGGTTCCCGGAAGAGGCGAGCTGAGCTGTCTGCAGGATGCCATCGATCACAGCGCC
TTCATCATTCTGCTGCTGACCAGCAACTTCGACTGCCGGCTGTCTCTGCACCAAGTGAACCAGGCCATGATGAGC
AACCTGACCAGACAGGGCAGCCCCGATTGCGTGATCCCATTCCTGCCACTGGAAAGCTCCCCAGCACAGCTGTCT
AGCGATACTGCCTCTCTGCTGTCTGGACTCGTGCGGCTGGATGAGCACAGCCAGATCTTCGCCAGAAAGGTGGCC
AACACCTTCAAGCCCCATCGGCTGCAGGCCAGAAAAGCCATGTGGtga
SEQ ID NO: 6
MACTGPSLPSAFDILGAAGQDKLLYLKHKLKTPRPGCQGQDLLHAMVLLKLGQETEARISLEALKADAVARLVAR
QWAGVDSTEDPEEPPDVSWAVARLYHLLAEEKLCPASLRDVAYQEAVRTLSSRDDHRLGELQDEARNRCGWDIAG
DPGSIRTLQSNLGCLPPSSALPSGTRSLPRPIDGVSDWSQGCSLRSTGSPASLASNLEISQSPTMPFLSLHRSPH
GPSKLCDDPQASLVPEPVPGGCQEPEEMSWPPSGEIASPPELPSSPPPGLPEVAPDATSTGLPDTPAAPETSTNY
PVECTEGSAGPQSLPLPILEPVKNPCSVKDQTPLQLSVEDTTSPNTKPCPPTPTTPETSPPPPPPPPSSTPCSAH
LTPSSLFPSSLESSSEQKFYNFVILHARADEHIALRVREKLEALGVPDGATFCEDFQVPGRGELSCLQDAIDHSA
FIILLLTSNFDCRLSLHQVNQAMMSNLTRQGSPDCVIPFLPLESSPAQLSSDTASLLSGLVRLDEHSQIFARKVA
NTFKPHRLQARKAMW-
hTRIF_PhosphoMutant “TRIF_PhosphoM”
Mutations for TBK1 phosphorylation sites (S210A, S212A, T214A)
SEQ ID NO: 7
ATGGCTTGCACAGGACCTTCTCTGCCCAGCGCCTTTGATATCCTGGGAGCCGCTGGACAGGACAAGCTGCTGTAC
CTGAAGCACAAGCTGAAAACCCCTCGGCCTGGCTGCCAGGGACAAGATCTGCTGCATGCTATGGTGCTGCTGAAG
CTGGGCCAAGAGACAGAGGCCAGAATCAGCCTGGAAGCCCTGAAGGCTGATGCCGTGGCTAGACTGGTTGCCAGA
CAATGGGCTGGCGTGGACAGCACAGAGGACCCTGAAGAACCTCCTGACGTGTCCTGGGCCGTCGCCAGACTGTAT
CATCTGCTGGCCGAAGAGAAGCTGTGCCCCGCCTCTCTGAGAGATGTGGCCTATCAAGAAGCCGTGCGGACCCTG
AGCAGCAGGGATGATCATAGACTGGGCGAGCTGCAGGACGAGGCCCGGAATAGATGTGGCTGGGATATTGCTGGC
GACCCCGGCAGCATTAGAACCCTGCAGTCTAACCTGGGCTGCCTGCCTCCATCTTCTGCCCTGCCATCTGGCACA
AGAAGCCTGCCTAGACCTATCGACGGCGTGTCCGATTGGAGCCAGGGCTGTTCTCTGAGAAGCACAGGATCTCCA
GCCAGCCTGGCCAGCAACCTGGAAATCgcaCAGgctCCTgccATGCCCTTCCTGAGCCTGCACAGAAGCCCTCAC
GGACCTAGCAAGCTGTGCGACGATCCTCAGGCTTCTCTGGTGCCTGAACCTGTTCCTGGCGGCTGCCAAGAGCCT
GAAGAGATGTCTTGGCCTCCTAGCGGCGAGATCGCCTCTCCACCTGAACTGCCATCTAGCCCTCCACCAGGACTG
CCTGAAGTGGCCCCTGATGCCACTTCTACAGGCCTGCCTGATACACCCGCCGCTCCAGAGACAAGCACAAACTAC
CCTGTGGAATGCACCGAGGGCTCTGCCGGACCTCAATCTCTGCCTCTGCCTATCCTGGAACCTGTGAAGAACCCT
TGCAGCGTGAAGGATCAGACCCCTCTGCAGCTGAGCGTGGAAGATACCACCTCTCCTAACACCAAGCCTTGTCCT
CCAACACCTACCACACCTGAGACAAGCCCACCTCCTCCGCCTCCACCACCAAGCTCTACACCTTGTAGCGCCCAC
CTGACACCAAGCAGCCTGTTTCCAAGCTCTCTGGAAAGCAGCAGCGAGCAGAAATTCTACAACTTCGTGATCCTG
CACGCCAGAGCCGACGAGCACATTGCCCTGAGAGTGCGCGAAAAGCTGGAAGCTCTGGGAGTGCCTGATGGCGCC
ACCTTCTGCGAGGATTTTCAGGTTCCCGGAAGAGGCGAGCTGAGCTGTCTGCAGGATGCCATCGATCACAGCGCC
TTCATCATTCTGCTGCTGACCAGCAACTTCGACTGCCGGCTGTCTCTGCACCAAGTGAACCAGGCCATGATGAGC
AACCTGACCAGACAGGGCAGCCCCGATTGCGTGATCCCATTCCTGCCACTGGAAAGCTCCCCAGCACAGCTGTCT
AGCGATACTGCCTCTCTGCTGTCTGGACTCGTGCGGCTGGATGAGCACAGCCAGATCTTCGCCAGAAAGGTGGCC
AACACCTTCAAGCCCCATCGGCTGCAGGCCAGAAAAGCCATGTGGCGGAAAGAGCAGGACACACGGGCACTGAGA
GAGCAGTCTCAGCACCTGGATGGCGAGAGAATGCAGGCCGCTGCTCTGAATGCCGCCTACAGCGCTTACCTGCAG
AGCTACCTGAGCTATCAGGCCCAGATGGAACAGCTGCAGGTCGCCTTTGGCAGCCACATGTCCTTTGGAACAGGC
GCCCCTTACGGCGCCAGAATGCCTTTTGGTGGACAGGTGCCACTGGGAGCCCCTCCACCTTTTCCAACATGGCCA
GGATGTCCCCAGCCTCCTCCACTGCATGCTTGGCAAGCTGGAACACCTCCGCCACCATCTCCACAGCCAGCTGCC
TTTCCACAGTCTCTCCCATTTCCACAGAGCCCCGCCTTTCCAACAGCTAGCCCTGCTCCTCCACAAAGCCCTGGA
CTGCAGCCCCTGATCATTCACCACGCACAGATGGTGCAGCTGGGACTGAACAATCACATGTGGAACCAGAGAGGC
TCTCAGGCCCCTGAGGACAAGACACAAGAGGCCGAAtga
SEQ ID NO: 8
MACTGPSLPSAFDILGAAGQDKLLYLKHKLKTPRPGCQGQDLLHAMVLLKLGQETEARISLEALKADAVARLVAR
QWAGVDSTEDPEEPPDVSWAVARLYHLLAEEKLCPASLRDVAYQEAVRTLSSRDDHRLGELQDEARNRCGWDIAG
DPGSIRTLQSNLGCLPPSSALPSGTRSLPRPIDGVSDWSQGCSLRSTGSPASLASNLEIAQAPAMPELSLHRSPH
GPSKLCDDPQASLVPEPVPGGCQEPEEMSWPPSGEIASPPELPSSPPPGLPEVAPDATSTGLPDTPAAPETSTNY
PVECTEGSAGPQSLPLPILEPVKNPCSVKDQTPLQLSVEDTTSPNTKPCPPTPTTPETSPPPPPPPPSSTPCSAH
LTPSSLFPSSLESSSEQKFYNFVILHARADEHIALRVREKLEALGVPDGATFCEDFQVPGRGELSCLQDAIDHSA
FIILLLTSNFDCRLSLHQVNQAMMSNLTRQGSPDCVIPFLPLESSPAQLSSDTASLLSGLVRLDEHSQIFARKVA
NTFKPHRLQARKAMWRKEQDTRALREQSQHLDGERMQAAALNAAYSAYLQSYLSYQAQMEQLQVAFGSHMSFGTG
APYGARMPFGGQVPLGAPPPFPTWPGCPQPPPLHAWQAGTPPPPSPQPAAFPQSLPFPQSPAFPTASPAPPQSPG
LQPLIIHHAQMVQLGLNNHMWNQRGSQAPEDKTQEAE
hTRIF_P434H “TRIF_P434H”
Mutation for dimerization site P434 in the TIR domain
SEQ ID NO: 9
ATGGCTTGCACAGGACCTTCTCTGCCCAGCGCCTTTGATATCCTGGGAGCCGCTGGACAGGACAAGCTGCTGTAC
CTGAAGCACAAGCTGAAAACCCCTCGGCCTGGCTGCCAGGGACAAGATCTGCTGCATGCTATGGTGCTGCTGAAG
CTGGGCCAAGAGACAGAGGCCAGAATCAGCCTGGAAGCCCTGAAGGCTGATGCCGTGGCTAGACTGGTTGCCAGA
CAATGGGCTGGCGTGGACAGCACAGAGGACCCTGAAGAACCTCCTGACGTGTCCTGGGCCGTCGCCAGACTGTAT
CATCTGCTGGCCGAAGAGAAGCTGTGCCCCGCCTCTCTGAGAGATGTGGCCTATCAAGAAGCCGTGCGGACCCTG
AGCAGCAGGGATGATCATAGACTGGGCGAGCTGCAGGACGAGGCCCGGAATAGATGTGGCTGGGATATTGCTGGC
GACCCCGGCAGCATTAGAACCCTGCAGTCTAACCTGGGCTGCCTGCCTCCATCTTCTGCCCTGCCATCTGGCACA
AGAAGCCTGCCTAGACCTATCGACGGCGTGTCCGATTGGAGCCAGGGCTGTTCTCTGAGAAGCACAGGATCTCCA
GCCAGCCTGGCCAGCAACCTGGAAATCAGCCAGTCTCCTACAATGCCCTTCCTGAGCCTGCACAGAAGCCCTCAC
GGACCTAGCAAGCTGTGCGACGATCCTCAGGCTTCTCTGGTGCCTGAACCTGTTCCTGGCGGCTGCCAAGAGCCT
GAAGAGATGTCTTGGCCTCCTAGCGGCGAGATCGCCTCTCCACCTGAACTGCCATCTAGCCCTCCACCAGGACTG
CCTGAAGTGGCCCCTGATGCCACTTCTACAGGCCTGCCTGATACACCCGCCGCTCCAGAGACAAGCACAAACTAC
CCTGTGGAATGCACCGAGGGCTCTGCCGGACCTCAATCTCTGCCTCTGCCTATCCTGGAACCTGTGAAGAACCCT
TGCAGCGTGAAGGATCAGACCCCTCTGCAGCTGAGCGTGGAAGATACCACCTCTCCTAACACCAAGCCTTGTCCT
CCAACACCTACCACACCTGAGACAAGCCCACCTCCTCCGCCTCCACCACCAAGCTCTACACCTTGTAGCGCCCAC
CTGACACCAAGCAGCCTGTTTCCAAGCTCTCTGGAAAGCAGCAGCGAGCAGAAATTCTACAACTTCGTGATCCTG
CACGCCAGAGCCGACGAGCACATTGCCCTGAGAGTGCGCGAAAAGCTGGAAGCTCTGGGAGTGCCTGATGGCGCC
ACCTTCTGCGAGGATTTTCAGGTTCatGGAAGAGGCGAGCTGAGCTGTCTGCAGGATGCCATCGATCACAGCGCC
TTCATCATTCTGCTGCTGACCAGCAACTTCGACTGCCGGCTGTCTCTGCACCAAGTGAACCAGGCCATGATGAGC
AACCTGACCAGACAGGGCAGCCCCGATTGCGTGATCCCATTCCTGCCACTGGAAAGCTCCCCAGCACAGCTGTCT
AGCGATACTGCCTCTCTGCTGTCTGGACTCGTGCGGCTGGATGAGCACAGCCAGATCTTCGCCAGAAAGGTGGCC
AACACCTTCAAGCCCCATCGGCTGCAGGCCAGAAAAGCCATGTGGCGGAAAGAGCAGGACACACGGGCACTGAGA
GAGCAGTCTCAGCACCTGGATGGCGAGAGAATGCAGGCCGCTGCTCTGAATGCCGCCTACAGCGCTTACCTGCAG
AGCTACCTGAGCTATCAGGCCCAGATGGAACAGCTGCAGGTCGCCTTTGGCAGCCACATGTCCTTTGGAACAGGC
GCCCCTTACGGCGCCAGAATGCCTTTTGGTGGACAGGTGCCACTGGGAGCCCCTCCACCTTTTCCAACATGGCCA
GGATGTCCCCAGCCTCCTCCACTGCATGCTTGGCAAGCTGGAACACCTCCGCCACCATCTCCACAGCCAGCTGCC
TTTCCACAGTCTCTCCCATTTCCACAGAGCCCCGCCTTTCCAACAGCTAGCCCTGCTCCTCCACAAAGCCCTGGA
CTGCAGCCCCTGATCATTCACCACGCACAGATGGTGCAGCTGGGACTGAACAATCACATGTGGAACCAGAGAGGC
TCTCAGGCCCCTGAGGACAAGACACAAGAGGCCGAAtga
SEQ ID NO: 10
MACTGPSLPSAFDILGAAGQDKLLYLKHKLKTPRPGCQGQDLLHAMVLLKLGQETEARISLEALKADAVARLVAR
QWAGVDSTEDPEEPPDVSWAVARLYHLLAEEKLCPASLRDVAYQEAVRTLSSRDDHRLGELQDEARNRCGWDIAG
DPGSIRTLQSNLGCLPPSSALPSGTRSLPRPIDGVSDWSQGCSLRSTGSPASLASNLEISQSPTMPFLSLHRSPH
GPSKLCDDPQASLVPEPVPGGCQEPEEMSWPPSGEIASPPELPSSPPPGLPEVAPDATSTGLPDTPAAPETSTNY
PVECTEGSAGPQSLPLPILEPVKNPCSVKDQTPLQLSVEDTTSPNTKPCPPTPTTPETSPPPPPPPPSSTPCSAH
LTPSSLFPSSLESSSEQKFYNFVILHARADEHIALRVREKLEALGVPDGATFCEDFQVHGRGELSCLQDAIDHSA
FIILLLTSNEDCRLSLHQVNQAMMSNLTRQGSPDCVIPFLPLESSPAQLSSDTASLLSGLVRLDEHSQIFARKVA
NTFKPHRLQARKAMWRKEQDTRALREQSQHLDGERMQAAALNAAYSAYLQSYLSYQAQMEQLQVAFGSHMSFGTG
APYGARMPFGGQVPLGAPPPFPTWPGCPQPPPLHAWQAGTPPPPSPQPAAFPQSLPFPQSPAFPTASPAPPQSPG
LQPLIIHHAQMVQLGLNNHMWNQRGSQAPEDKTQEAE
miniTRIF “miniTRIF”
N-ter deletion (1-311)
SEQ ID NO: 11
ATGCAGTCTCTGCCCCTGCCTATCCTGGAACCTGTGAAGAACCCCTGCAGCGTGAAGGATCAGACCCCTCTGCAG
CTGAGCGTGGAAGATACCACCTCTCCTAACACCAAGCCTTGTCCTCCAACACCTACCACACCTGAGACAAGCCCT
CCACCTCCGCCTCCACCACCAAGCTCTACACCTTGTAGCGCCCACCTGACACCAAGCAGCCTGTTTCCATCCAGC
CTGGAAAGCAGCAGCGAGCAGAAATTCTACAACTTCGTGATCCTGCACGCCAGAGCCGACGAGCACATTGCCCTG
AGAGTGCGCGAAAAGCTGGAAGCCCTGGGAGTTCCTGATGGCGCCACCTTCTGCGAGGATTTCCAAGTGCCTGGA
AGAGGCGAGCTGAGCTGTCTGCAGGATGCCATCGATCACAGCGCCTTCATCATCCTGCTGCTGACCAGCAACTTC
GACTGCAGACTGAGCCTGCACCAAGTGAACCAGGCCATGATGAGCAACCTGACCAGACAGGGCAGCCCCGATTGC
GTGATCCCATTCCTGCCTCTGGAAAGCTCCCCTGCTCAGCTGTCTAGCGATACAGCCTCTCTGCTGTCTGGACTC
GTGCGGCTGGATGAGCACAGCCAGATCTTCGCCAGAAAGGTGGCCAACACCTTCAAGCCCCATCGGCTGCAGGCC
AGAAAAGCCATGTGGCGGAAAGAGCAGGACACACGGGCTCTGAGAGAGCAGTCTCAGCACCTGGATGGCGAGAGA
ATGCAGGCCGCTGCTCTGAATGCCGCCTACTCTGCCTACCTGCAGAGCTACCTGAGCTATCAGGCCCAGATGGAA
CAGCTGCAGGTCGCCTTTGGCAGCCACATGTCCTTTGGAACAGGCGCCCCTTACGGCGCCAGAATGCCTTTTGGT
GGACAGGTGCCACTGGGAGCCCCTCCACCATTTCCTACATGGCCAGGATGTCCCCAGCCTCCTCCACTGCATGCT
TGGCAAGCTGGAACACCACCTCCTCCATCTCCACAGCCTGCCGCCTTTCCACAGAGCCTGCCTTTTCCACAGTCT
CCCGCTTTTCCCACCGCCTCTCCAGCTCCACCACAATCTCCAGGACTGCAGCCCCTGATCATCCACCACGCTCAA
ATGGTGCAGCTGGGACTGAACAATCACATGTGGAACCAGCGGGGCTCTCAGGCCCCTGAGGATAAGACACAAGAG
GCCGAGTGA
SEQ ID NO: 12
MQSLPLPILEPVKNPCSVKDQTPLQLSVEDTTSPNTKPCPPTPTTPETSPPPPPPPPSSTPCSAHLTPSSLFPSS
LESSSEQKFYNFVILHARADEHIALRVREKLEALGVPDGATFCEDFQVPGRGELSCLQDAIDHSAFIILLLTSNE
DCRLSLHQVNQAMMSNLTRQGSPDCVIPFLPLESSPAQLSSDTASLLSGLVRLDEHSQIFARKVANTFKPHRLQA
RKAMWRKEQDTRALREQSQHLDGERMQAAALNAAYSAYLQSYLSYQAQMEQLQVAFGSHMSFGTGAPYGARMPEG
GQVPLGAPPPFPTWPGCPQPPPLHAWQAGTPPPPSPQPAAFPQSLPFPQSPAFPTASPAPPQSPGLQPLIIHHAQ
MVQLGLNNHMWNQRGSQAPEDKTQEAE-
hTRIF_d1-180_ “TRIF_d1-180”
N-ter deletion (1-180)
SEQ ID NO: 13
ATGCCCATCGACGGCGTGTCCGATTGGAGCCAGGGCTGTTCTCTGCGGAGCACCGGCAGCCCTGCCAGCCTGGCC
TCTAACCTGGAAATCAGCCAAAGCCCCACCATGCCATTCCTGTCTCTTCACAGAAGTCCGCACGGCCCTAGCAAG
CTGTGCGACGACCCCCAGGCTAGCCTGGTGCCAGAACCTGTGCCTGGCGGCTGCCAGGAGCCTGAGGAAATGAGC
TGGCCTCCTTCTGGCGAGATCGCCTCCCCGCCCGAGCTGCCTAGCTCTCCTCCTCCCGGCCTGCCTGAAGTGGCT
CCTGATGCCACTTCTACAGGACTGCCTGATACACCCGCCGCACCCGAAACCAGCACCAACTACCCCGTGGAATGC
ACCGAGGGCTCAGCCGGCCCCCAATCTCTCCCTCTGCCTATCCTGGAGCCCGTGAAAAACCCTTGCAGCGTGAAG
GACCAGACCCCTCTGCAGCTGAGCGTGGAGGATACCACAAGCCCCAACACAAAGCCCTGTCCCCCTACCCCCACA
ACACCTGAGACAAGCCCACCACCTCCTCCTCCACCTCCTAGCAGTACCCCTTGTAGCGCCCATCTGACCCCATCA
AGCCTGTTCCCTAGCTCTCTAGAGAGCAGCAGTGAACAAAAGTTCTACAACTTCGTGATCCTGCACGCCAGAGCC
GACGAGCACATCGCCCTGAGAGTGCGGGAAAAGCTGGAGGCCCTGGGTGTGCCTGACGGCGCCACCTTCTGCGAG
GACTTCCAGGTCCCTGGCAGAGGCGAGCTGTCCTGTCTGCAAGACGCCATCGACCACTCTGCCTTCATCATCCTG
CTGCTGACCTCCAATTTCGACTGCCGGCTGAGCCTCCACCAGGTTAATCAGGCCATGATGAGCAACCTGACAAGA
CAGGGCAGCCCCGACTGCGTGATCCCTTTCCTGCCTCTGGAAAGCAGCCCTGCCCAACTGTCGTCTGATACAGCC
AGCCTGCTGAGCGGCCTGGTCAGACTGGACGAGCACAGCCAGATTTTCGCCAGAAAAGTGGCCAACACCTTCAAG
CCTCACCGGCTGCAGGCCCGGAAAGCCATGTGGCGGAAGGAACAGGACACCAGAGCCCTGAGAGAGCAGAGCCAG
CACCTGGATGGCGAAAGAATGCAGGCCGCTGCCCTGAATGCCGCCTACAGCGCCTACCTGCAGAGCTACCTGTCT
TATCAGGCCCAGATGGAACAGCTGCAGGTGGCTTTTGGCAGCCACATGTCCTTCGGCACCGGCGCTCCTTACGGT
GCCAGGATGCCCTTCGGAGGCCAGGTGCCACTGGGAGCCCCTCCTCCTTTTCCTACCTGGCCCGGCTGCCCCCAA
CCTCCACCTCTGCATgcttggcaagctggaacacctccaccaccttctccaaAGCCTGCCGCATTTCCCCAGTCC
CTGCCTTTTCCTCAGAGCCCTGCTTTCCCAACCGCCAGCCCAGCTCCTCCCCAGAGCCCTGGACTGCAGCCTCTG
ATCATCCACCACGCCCAGATGGTGCAGCTCGGACTGAACAACCACATGTGGAACCAGAGAGGCTCTCAGGCTCCT
GAGGACAAGACACAGGAGGCCGAGTGA
SEQ ID NO: 14
MPIDGVSDWSQGCSLRSTGSPASLASNLEISQSPTMPFLSLHRSPHGPSKLCDDPQASLVPEPVPGGCQEPEEMS
WPPSGEIASPPELPSSPPPGLPEVAPDATSTGLPDTPAAPETSTNYPVECTEGSAGPQSLPLPILEPVKNPCSVK
DQTPLQLSVEDTTSPNTKPCPPTPTTPETSPPPPPPPPSSTPCSAHLTPSSLFPSSLESSSEQKFYNFVILHARA
DEHIALRVREKLEALGVPDGATFCEDFQVPGRGELSCLQDAIDHSAFIILLLTSNFDCRLSLHQVNQAMMSNLTR
QGSPDCVIPFLPLESSPAQLSSDTASLLSGLVRLDEHSQIFARKVANTFKPHRLQARKAMWRKEQDTRALREQSQ
HLDGERMQAAALNAAYSAYLQSYLSYQAQMEQLQVAFGSHMSFGTGAPYGARMPFGGQVPLGAPPPFPTWPGCPQ
PPPLHAWQAGTPPPPSPKPAAFPQSLPFPQSPAFPTASPAPPQSPGLQPLIIHHAQMVQLGLNNHMWNQRGSQAP
EDKTQEAE
TIRdomain_ “TIR domain”
Fragment 387-544
SEQ ID NO: 15
ATGGAGTCTAGCAGCGAGCAGAAATTCTACAACTTCGTGATCCTGCACGCCAGAGCCGACGAGCACATTGCCCTG
AGAGTGCGCGAAAAGCTGGAAGCCCTGGGAGTTCCTGATGGCGCCACCTTCTGCGAGGATTTCCAAGTGCCTGGA
AGAGGCGAGCTGAGCTGTCTGCAGGATGCCATCGATCACAGCGCCTTCATCATCCTGCTGCTGACCAGCAACTTC
GACTGCAGACTGAGCCTGCACCAAGTGAACCAGGCCATGATGAGCAACCTGACCAGACAGGGCAGCCCCGATTGC
GTGATCCCATTCCTGCCTCTGGAAAGCAGCCCTGCTCAGCTGTCTAGCGATACAGCCTCTCTGCTGTCTGGACTC
GTGCGGCTGGATGAGCACAGCCAGATCTTCGCCAGAAAGGTGGCCAACACCTTCAAGCCCCATCGGCTGCAGGCC
AGAAAAGCCATGTGGCGGAAAGAGCAGGACtga
SEQ ID NO: 16
MESSSEQKFYNFVILHARADEHIALRVREKLEALGVPDGATFCEDFQVPGRGELSCLQDAIDHSAFIILLLTSNF
DCRLSLHQVNQAMMSNLTRQGSPDCVIPFLPLESSPAQLSSDTASLLSGLVRLDEHSQIFARKVANTFKPHRLQA
RKAMWRKEQ-
TRIS_d1-180 “TRIS”
Deletion of N-ter fragment 1-180 and fragment 217-658
SEQ ID NO: 17
ATGCCTATCGACGGCGTGTCCGACTGGTCCCAAGGCTGCAGCCTGAGAAGCACCGGCAGCCCTGCTAGCCTGGCC
TCTAATCTGGAAATCAGCCAATCTCCAACAATGCCCCAGAGCCCTGCCTTCCCCACCGCCAGCCCAGCTCCTCCT
CAGTCTCCCGGCCTGCAGCCTCTGATCATCCACCACGCCCAGATGGTGCAGCTGGGCCTGAACAACCACATGTGG
AACCAGCGGGGAAGCCAGGCCCCTGAGGACAAAACCCAGGAGGCCGAATGA
SEQ ID NO: 18
MPIDGVSDWSQGCSLRSTGSPASLASNLEISQSPTMPQSPAFPTASPAPPQSPGLQPLIIHHAQMVQLGLNNHMW
NQRGSQAPEDKTQEAE-
TRIR “TRIR”
Deletion of N-ter fragment 1-180, fragment 217-386 and fragment 546-712
SEQ ID NO: 19
ATGCCTATCGACGGAGTGTCCGACTGGTCCCAGGGATGTAGCCTTAGAAGCACCGGCAGCCCTGCTAGCCTGGCC
TCTAACCTGGAAATCAGCCAGAGCCCTACAATGCCAGAGAGCAGCAGCGAGCAGAAATTCTACAACTTCGTGATT
CTGCACGCCAGAGCCGACGAGCACATCGCCCTGAGAGTGCGGGAAAAGCTGGAGGCCCTGGGCGTGCCTGATGGC
GCCACCTTCTGCGAAGATTTTCAGGTCCCCGGCAGAGGCGAGCTGAGCTGCCTGCAGGACGCTATCGACCACAGC
GCCTTCATCATCCTGCTGCTGACAAGCAACTTCGACTGCAGGCTGAGCCTGCACCAGGTGAACCAGGCCATGATG
AGCAATCTGACCAGACAGGGCTCCCCAGATTGCGTGATCCCCTTCCTGCCTCTGGAGAGCTCTCCTGCACAGCTG
TCTTCTGACACCGCCAGCCTGCTCTCCGGCCTGGTGCGGCTGGACGAGCATTCTCAAATCTTCGCCAGAAAAGTG
GCCAACACCTTTAAGCCCCACAGACTGCAAGCTCGGAAGGCCATGTGGCGGAAGGAACAGGATTGA
SEQ ID NO: 20
MPIDGVSDWSQGCSLRSTGSPASLASNLEISQSPTMPESSSEQKFYNFVILHARADEHIALRVREKLEALGVPDG
ATFCEDFQVPGRGELSCLQDAIDHSAFIILLLTSNFDCRLSLHQVNQAMMSNLTRQGSPDCVIPFLPLESSPAQL
SSDTASLLSGLVRLDEHSQIFARKVANTFKPHRLQARKAMWRKEQ
TRIR_RIPK3 “TRIR3”
TRIR followed by a flexible linker GPGGSSGSS and hRIPK3 (UniProtKB-Q9Y572
(RIPK3 HUMAN))
SEQ ID NO: 21
ATGCCTATCGACGGCGTGTCCGATTGGAGCCAGGGATGTTCTCTGCGGAGCACAGGCTCTCCCGCCTCTCTGGCC
AGCAACCTGGAAATCAGCCAATCTCCTACCATGCCCGAAAGCTCTTCTGAGCAGAAGTTCTACAACTTTGTGATC
CTGCACGCCCGGGCCGACGAGCACATCGCCCTGAGAGTGCGGGAAAAGCTGGAAGCCCTGGGCGTGCCCGACGGC
GCAACATTTTGTGAAGATTTCCAGGTTCCTGGTAGAGGAGAGCTTTCCTGTCTGCAAGACGCCATTGACCACAGC
GCCTTCATCATCCTGCTGCTGACATCTAACTTCGACTGCAGACTGTCCCTGCACCAGGTGAATCAGGCCATGATG
AGCAATCTGACCAGACAGGGCAGCCCTGACTGCGTGATCCCCTTCCTGCCACTGGAAAGCAGCCCTGCTCAGCTG
AGTAGCGACACCGCCAGCCTGCTCAGCGGCCTGGTCAGACTGGACGAACACTCTCAAATCTTCGCCAGAAAAGTG
GCTAATACCTTCAAGCCCCACCGGCTGCAGGCCAGAAAGGCTATGTGGCGGAAAGAACAGGATGGCCCCGGCGGA
TCCAGCGGCAGCAGCTGCGTCAAACTGTGGCCTAGCGGCGCCCCTGCCCCTCTGGTGTCCATCGAGGAACTGGAA
AACCAAGAGCTTGTCGGCAAGGGCGGCTTCGGCACTGTTTTCAGAGCTCAACACAGAAAGTGGGGCTACGACGTG
GCCGTGAAGATCGTGAACAGCAAGGCCATCAGCAGAGAAGTGAAGGCCATGGCCAGCCTGGACAACGAGTTCGTG
CTGAGACTGGAGGGAGTGATCGAGAAGGTGAACTGGGACCAGGATCCTAAACCCGCCCTGGTGACAAAGTTCATG
GAAAACGGCAGCCTGAGCGGACTGCTGCAGAGCCAATGTCCTAGACCTTGGCCCCTGCTGTGCCGGCTCCTGAAG
GAAGTGGTGCTCGGCATGTTCTACCTGCATGATCAGAATCCCGTGCTGCTGCACAGAGATCTGAAACCCTCCAAC
GTGCTGCTGGATCCTGAGCTGCACGTGAAGCTGGCCGATTTCGGCCTGAGCACCTTTCAGGGCGGCAGTCAGAGC
GGCACAGGCAGCGGCGAGCCTGGCGGCACCCTGGGCTACCTGGCTCCTGAGCTGTTCGTGAATGTGAATCGGAAG
GCCAGCACAGCTTCTGACGTGTATAGCTTTGGCATCCTGATGTGGGCCGTGCTGGCTGGAAGGGAAGTTGAGCTG
CCCACCGAGCCCAGCCTGGTGTACGAGGCCGTGTGCAACCGGCAGAACCGGCCAAGCCTGGCCGAGCTGCCTCAG
GCTGGCCCTGAGACACCTGGCCTTGAGGGCCTGAAGGAGCTCATGCAGCTGTGCTGGAGCTCCGAGCCAAAGGAC
AGACCATCTTTCCAGGAGTGCCTGCCTAAGACCGACGAGGTGTTCCAGATGGTGGAAAACAACATGAACGCCGCC
GTCAGCACCGTGAAGGACTTTCTGAGCCAGCTGAGATCTTCCAATAGACGCTTCAGCATCCCTGAGTCTGGACAG
GGAGGAACAGAGATGGACGGATTCCGGAGAACCATCGAGAACCAGCACTCCCGGAACGACGTGATGGTCAGCGAG
TGGCTGAACAAGCTGAACCTGGAAGAGCCTCCAAGCTCAGTGCCCAAGAAGTGCCCCTCTCTGACCAAAAGAAGC
AGAGCCCAGGAGGAACAGGTGCCTCAGGCCTGGACCGCCGGAACAAGCAGCGACAGCATGGCCCAGCCTCCGCAA
ACACCTGAAACCAGCACCTTCAGAAACCAGATGCCTAGCCCCACCAGCACCGGCACCCCTTCCCCTGGCCCCAGA
GGCAACCAGGGCGCAGAGCGGCAGGGCATGAACTGGTCCTGCCGCACCCCCGAGCCCAACCCTGTGACCGGCCGG
CCTCTGGTGAACATCTACAACTGCAGCGGTGTGCAGGTGGGTGATAACAATTACCTGACCATGCAGCAGACCACC
GCTCTGCCAACATGGGGCCTCGCCCCTTCTGGCAAGGGCAGAGGCCTGCAGCATCCTCCTCCTGTGGGCTCTCAG
GAAGGACCTAAGGACCCCGAGGCCTGGAGCAGGCCTCAGGGCTGGTACAACCACTCAGGCAAATGA
SEQ ID NO: 22
MPIDGVSDWSQGCSLRSTGSPASLASNLEISQSPTMPESSSEQKFYNFVILHARADEHIALRVREKLEALGVPDG
ATFCEDFQVPGRGELSCLQDAIDHSAFIILLLTSNEDCRLSLHQVNQAMMSNLTRQGSPDCVIPFLPLESSPAQL
SSDTASLLSGLVRLDEHSQIFARKVANTFKPHRLQARKAMWRKEQDGPGGSSGSSCVKLWPSGAPAPLVSIEELE
NQELVGKGGFGTVFRAQHRKWGYDVAVKIVNSKAISREVKAMASLDNEFVLRLEGVIEKVNWDQDPKPALVTKEM
ENGSLSGLLQSQCPRPWPLLCRLLKEVVLGMFYLHDQNPVLLHRDLKPSNVLLDPELHVKLADFGLSTFQGGSQS
GTGSGEPGGTLGYLAPELFVNVNRKASTASDVYSFGILMWAVLAGREVELPTEPSLVYEAVCNRQNRPSLAELPQ
AGPETPGLEGLKELMQLCWSSEPKDRPSFQECLPKTDEVFQMVENNMNAAVSTVKDFLSQLRSSNRRFSIPESGQ
GGTEMDGFRRTIENQHSRNDVMVSEWINKLNLEEPPSSVPKKCPSLTKRSRAQEEQVPQAWTAGTSSDSMAQPPQ
TPETSTERNQMPSPTSTGTPSPGPRGNQGAERQGMNWSCRTPEPNPVTGRPLVNIYNCSGVQVGDNNYLTMQQTT
ALPTWGLAPSGKGRGLQHPPPVGSQEGPKDPEAWSRPQGWYNHSGK
FLAG-tag
SEQ ID NO: 23
gactacaaggacgacgacgacaag
SEQ ID NO: 24
DYKDDDDK
Linker
SEQ ID NO: 25
GPGGSSGSS
T2A peptide
SEQ ID NO: 26
EGRGSLLTCGDVEENPGP
P2A peptide
SEQ ID NO: 27
ATNFSLLKQAGDVEENPGP
E2A peptide
SEQ ID NO: 28
QCTNYALLKLAGDVESNPGP
P2A peptide
SEQ ID NO: 29
VKQTLNFDLLKLAGDVESNPGP
Human wildtype RIPK3 amino acid sequence
SEQ ID NO: 30
MSCVKLWPSGAPAPLVSIEELENQELVGKGGFGTVFRAQHRKWGYDVAVKIVNSKAISRE
VKAMASLDNEFVLRLEGVIEKVNWDQDPKPALVTKEMENGSLSGLLQSQCPRPWPLLCRL
LKEVVLGMFYLHDQNPVLLHRDLKPSNVLLDPELHVKLADFGLSTFQGGSQSGTGSGEPG
GTLGYLAPELFVNVNRKASTASDVYSFGILMWAVLAGREVELPTEPSLVYEAVCNRQNRP
SLAELPQAGPETPGLEGLKELMQLCWSSEPKDRPSFQECLPKTDEVFQMVENNMNAAVST
VKDFLSQLRSSNRRESIPESGQGGTEMDGFRRTIENQHSRNDVMVSEWLNKLNLEEPPSS
VPKKCPSLTKRSRAQEEQVPQAWTAGTSSDSMAQPPQTPETSTERNQMPSPTSTGTPSPG
PRGNQGAERQGMNWSCRTPEPNPVTGRPLVNIYNCSGVQVGDNNYLTMQQTTALPTWGLA
PSGKGRGLQHPPPVGSQEGPKDPEAWSRPQGWYNHSGK
Human wildtype RIPK3 nucleic acid sequence
SEQ ID NO: 31
atgtcgtgcg tcaagttatg gcccagcggt gcccccgccc ccttggtgtc catcgaggaa 60
ctggagaacc aggagctcgt cggcaaaggc gggttcggca cagtgttccg ggcgcaacat 120
aggaagtggg gctacgatgt ggcggtcaag atcgtaaact cgaaggcgat atccagggag 180
gtcaaggcca tggcaagtct ggataacgaa ttcgtgctgc gcctagaagg ggttatcgag 240
aaggtgaact gggaccaaga toccaagccg gctctggtga ctaaattcat ggagaacggc 300
tccttgtcgg ggctgctgca gtcccagtgc cctcggccct ggccgctcct ttgccgcctg 360
ctgaaagaag tggtgcttgg gatgttttac ctgcacgacc agaacccggt gctcctgcac 420
cgggacctca agccatccaa cgtcctgctg gacccagagc tgcacgtcaa gctggcagat 480
tttggcctgt ccacatttca gggaggctca cagtcaggga cagggtccgg ggagccaggg 540
ggcaccctgg gctacttggc cccagaactg tttgttaacg taaaccggaa ggcctccaca 600
gccagtgacg tctacagctt cgggatccta atgtgggcag tgcttgctgg aagagaagtt 660
gagttgccaa ccgaaccatc actcgtgtac gaagcagtgt gcaacaggca gaaccggcct 720
tcattggctg agctgcccca agccgggcct gagactcccg gottagaagg actgaaggag 780
ctaatgcagc tctgctggag cagtgagccc aaggacagac cctccttcca ggaatgccta 840
ccaaaaactg atgaagtctt ccagatggtg gagaacaata tgaatgctgc tgtctccacg 900
gtaaaggatt tcctgtctca gctcaggagc agcaatagga gattttctat cccagagtca 960
ggccaaggag ggacagaaat ggatggcttt aggagaacca tagaaaacca gcactctcgt 1020
aatgatgtca tggtttctga gtggctaaac aaactgaatc tagaggagcc tcccagctct 1080
gttcctaaaa aatgcccgag ccttaccaag aggagcaggg cacaagagga gcaggttcca 1140
caagcctgga cagcaggcac atcttcagat tcgatggccc aacctcccca gactccagag 1200
acctcaactt tcagaaacca gatgcccagc cctacctcaa ctggaacacc aagtcctgga 1260
ccccgaggga atcagggggc tgagagacaa ggcatgaact ggtcctgcag gaccccggag 1320
ccaaatccag taacagggcg accgctcgtt aacatataca actgctctgg ggtgcaagtt 1380
ggagacaaca actacttgac tatgcaacag acaactgcct tgcccacatg gggcttggca 1440
ccttcgggca aggggagggg cttgcagcac cccccaccag taggttcgca agaaggccct 1500
aaagatcctg aagcctggag caggccacag ggttggtata atcatagcgg gaaataa 1557
VICA amino acid sequence
SEQ ID NO: 32
MDDLRDTLMAYGCIAIRAGDENGLNDFLEQECGTRLHVAWPERCFIQLRSRSALGPFVGK
MGTVCSQGAYVCCQEYLHPFGFVEGPGFMRYQLIVLIGQRGGIYCYDDLRDCIYELAPTM
KDFLRHGFRHCDHFHTMRDYQRPMVQYDDYWNAVMLYRGDVESLSAEVTKRGYASYSIDD
PFDECPDTHFAFWTHNTEVMKFKETSFSVVRAGGSIQTMELMIRTVPRITCYHQLLGALG
HEVPERKEFLVRQYVLVDTFGVVYGYDPAMDAVYRLAEDVVMFTCVMGKKGHRNHRESGR
REAIVRLEKTPTCQHPKKTPDPMIMEDEDDDDELSLPRNVMTHEEAESRLYDAITENLMH
CVKLVTTDSPLATHLWPQELQALCDSPALSLCTDDVEGVRQKLRARTGSLHHFELSYRFH
DEDPETYMGFLWDIPSCDRCVRRRRFKVCDVGRRHIIPGAANGMPPLTPPHAYMNN
5′ UTR
SEQ ID NO: 33
GGGAAAUAAGAGAGAAAAGAAGAGUAAGAAGAAAUAUAAGACCCCGGCGCCGCCACC
3′ UTR DNA sequence, Variation 1
SEQ ID NO: 34
UAAUGAUAAUAGGCUGGAGCCUCGGUGGCCAUGCUUCUUGCCCCUUGGGCCUCCCCCCAGCCCCUCCUCCCCUUC
CUGCACCCGUACCCCCCAAACACCAUUGUCACACUCCAGUGGUCUUUGAAUAAAGUCUGAGUGGGGGCGCGGCC
GC
miR122 binding site is shown in bold and underlined
3′ UTR DNA sequence, Variation 2
SEQ ID NO: 35
UGAUAAUAGUCCAUAAAGUAGGAAACACUACAGCUGGAGCCUCGGUGGCCAUGCUUCUUGCCCCUUGGGCCCAAA
CACCAUUGUCACACUCCAUCCCCCCAGCCCCUCCUCCCCUUCCUGCACCCGUACCCCCGUGGUCUUUGAAUAAAG
UCUGAGUGGGCGGCGCGGCCGC
miR142 binding site is underlined, miR122 binding site is shown in bold and
underlined
miR-122-5p binding site RNA sequence
SEQ ID NO: 36
CAAACACCAUUGUCACACUCCA
miR-142-3p binding site RNA sequence
SEQ ID NO: 37
UCCAUAAAGUAGGAAACACUACA
PolyA tail
SEQ ID NO: 38
AAAAAAAAAAAAAAAAAAAAAAAAAAAAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAAAAAAAAAAAA
Human TRIF (hTRIF) Construct DNA sequence
SEQ ID NO: 39
GGGAAATAAGAGAGAAAAGAAGAGTAAGAAGAAATATAAGACCCCGGCGCCGCCACCATGGCCTGTACCG
GCCCCTCCCTCCCCTCGGCCTTCGACATCCTGGGAGCAGCGGGCCAAGACAAGCTCCTGTACCTGAAGCA
CAAGCTGAAAACTCCTAGGCCCGGATGTCAGGGCCAGGATCTGCTGCACGCGATGGTGCTGCTCAAGCTG
GGTCAAGAAACCGAGGCCCGCATCAGCCTGGAAGCCTTGAAAGCGGACGCCGTGGCCAGGCTCGTGGCCC
GGCAGTGGGCCGGAGTGGACTCCACTGAGGATCCGGAAGAACCCCCGGATGTGTCGTGGGCGGTGGCTCG
CCTGTACCACCTCCTGGCTGAAGAGAAGTTATGCCCAGCCTCCCTCCGCGACGTGGCATACCAGGAGGCC
GTGCGGACCCTCTCCTCGCGGGACGACCATCGCCTCGGTGAACTGCAGGATGAAGCTCGCAACAGATGTG
GCTGGGATATTGCTGGCGATCCGGGATCCATTCGGACACTGCAGAGCAACCTTGGATGCCTGCCCCCATC
GTCCGCGCTGCCGAGCGGCACCCGATCCCTGCCACGCCCGATTGACGGAGTCTCCGACTGGTCACAGGGA
TGCAGCCTCCGGTCCACCGGATCCCCGGCCTCACTCGCAAGCAACCTGGAAATCTCGCAGTCCCCAACTA
TGCCGTTCCTGTCACTGCATCGCTCCCCTCATGGACCATCCAAGCTGTGCGACGATCCACAGGCCTCCCT
CGTGCCTGAACCTGTGCCGGGAGGCTGCCAGGAACCGGAGGAGATGTCCTGGCCGCCTAGCGGGGAGATC
GCCTCACCGCCTGAACTCCCGTCCTCCCCCCCTCCGGGTTTGCCCGAAGTGGCCCCTGACGCGACCAGCA
CCGGACTGCCCGACACTCCCGCCGCGCCCGAAACCTCCACTAATTACCCGGTGGAATGCACCGAGGGGTC
GGCAGGGCCACAGTCCCTGCCTCTGCCTATTCTGGAGCCTGTGAAGAATCCCTGCTCTGTGAAGGACCAG
ACTCCTCTGCAACTGTCAGTGGAGGATACCACCTCGCCCAACACGAAGCCCTGCCCTCCTACTCCAACTA
CCCCTGAGACTTCGCCTCCGCCGCCTCCCCCGCCACCCTCCTCCACCCCGTGTTCCGCCCACCTCACACC
TTCCTCCCTGTTTCCGTCATCCCTGGAAAGTTCGTCGGAACAGAAATTCTACAACTTCGTGATCCTCCAC
GCGCGGGCTGATGAGCATATCGCCTTGCGCGTGCGGGAGAAGTTGGAAGCACTGGGGGTGCCCGACGGTG
CCACCTTCTGCGAGGATTTTCAGGTCCCCGGAAGGGGAGAGCTCAGCTGCCTGCAAGACGCCATCGACCA
CAGCGCCTTCATCATCCTCCTTCTGACCTCAAACTTCGACTGCCGCCTGAGCCTGCACCAGGTCAACCAG
GCCATGATGTCCAACCTGACCAGACAGGGCTCACCCGACTGTGTGATTCCGTTCCTGCCACTGGAGAGCA
GCCCCGCCCAGCTGTCCTCCGACACCGCCTCACTACTGTCCGGCCTTGTCCGGCTGGATGAACACAGCCA
GATCTTCGCAAGAAAGGTCGCGAACACCTTCAAGCCGCACAGACTGCAGGCCCGGAAGGCCATGTGGAGA
AAGGAGCAGGACACGAGAGCCCTTCGGGAGCAGTCCCAGCATCTTGACGGGGAACGCATGCAGGCCGCTG
CCTTGAACGCGGCGTACAGCGCCTATCTGCAATCGTACCTGTCCTACCAAGCACAGATGGAGCAGCTCCA
AGTGGCTTTCGGAAGCCACATGAGCTTTGGAACTGGTGCACCGTACGGCGCCAGGATGCCCTTCGGCGGA
CAAGTCCCTCTGGGTGCCCCCCCACCGTTCCCAACCTGGCCCGGGTGCCCTCAGCCGCCCCCGCTTCACG
CTTGGCAGGCCGGCACTCCTCCTCCCCCGTCCCCTCAACCGGCCGCTTTCCCGCAATCCCTTCCCTTCCC
ACAATCTCCCGCCTTCCCGACTGCCTCCCCGGCCCCGCCTCAATCCCCGGGACTCCAGCCTTTGATCATT
CACCACGCGCAAATGGTCCAGCTGGGGCTGAACAACCATATGTGGAACCAGCGGGGCTCGCAGGCCCCCG
AGGACAAGACCCAGGAAGCAGAGTAATGATAATAGGCTGGAGCCTCGGTGGCCATGCTTCTTGCCCCTTG
GGCCTCCCCCCAGCCCCTCCTCCCCTTCCTGCACCCGTACCCCCCAAACACCATTGTCACACTCCAGTGG
TCTTTGAATAAAGTCTGAGTGGGCGGCGCGGCCGCAAAAAAAAAAAAAAAAAAAAAAAAAAAAAAAAAAA
AAAAAAAAAAAAAAAAAAAAAAAAAAAAAAAAAAAAAAAAAAAAAAAAAAAAAAAAAAAAAAAAAAAAAA
AAAAAAAAAAAAAAAAAAAAAAAAA
Alternative 3′UTR
(SEQ ID NO: 68)
TGATAATAGTCCATAAAGTAGGAAACACTACAGCTGGAGCCTCGGTGGCCATGCTTCTTGCCCCTTGGGC
CCAAACACCATTGTCACACTCCATCCCCCCAGCCCCTCCTCCCCTTCCTGCACCCGTACCCCCGTGGTCT
TTGAATAAAGTCTGAGTGGGCGGCGCGGCCGCAAAAAAAAAAAAAAAAAAAAAAAAAAAAAAAAAAAAAA
AAAAAAAAAAAAAAAAAAAAAAAAAAAAAAAAAAAAAAAAAAAAAAAAAAAAAAAAAAAAAAAAAAAAAA
AAAAAAAAAAAAAAAAAAAAAA
5′ UTR-underlined
TRIF coding region-plain text
3′ UTR-italic
miR122 (CAAACACCATTGTCACACTCCA)-bold, underlined, italic
miR142 (TCCATAAAGTAGGAAACACTACA)-bold, underlined, italic
polyA tail-bold
Human Gasdermin E (hGSDME) Construct DNA Sequence
SEQ ID NO: 40
GGGAAATAAGAGAGAAAAGAAGAGTAAGAAGAAATATAAGACCCCGGCGCCGCCACCATGTTCGCCAAGG
CCACCAGAAACTTCCTGAGGGAAGTGGACGCCGACGGGGACTTGATTGCGGTGTCCAACCTCAACGACTC
CGACAAGCTCCAGCTGCTTAGCCTGGTCACCAAGAAGAAGCGGTTCTGGTGCTGGCAGCGGCCCAAGTAC
CAGTTCCTGTCGCTGACACTAGGCGACGTCTTGATAGAGGACCAGTTCCCCTCCCCGGTGGTCGTGGAAT
CCGATTTCGTGAAATATGAGGGGAAGTTCGCCAATCACGTGTCCGGAACTCTGGAAACTGCCCTGGGAAA
GGTCAAGTTGAACCTGGGCGGAAGCTCTAGGGTGGAATCGCAGTCCTCCTTTGGTACCCTGCGCAAGCAG
GAAGTGGATCTGCAGCAGCTCATTCGGGATAGCGCCGAGCGCACCATCAACCTGAGAAACCCCGTGCTCC
AACAAGTGCTGGAAGGGAGAAACGAAGTGCTCTGTGTGCTGACTCAGAAGATCACTACGATGCAGAAATG
CGTGATCTCAGAGCATATGCAAGTCGAGGAGAAATGCGGAGGAATCGTAGGAATTCAGACCAAGACTGTC
CAAGTGTCCGCCACTGAGGACGGAAACGTGACCAAGGACTCAAACGTGGTGCTGGAAATCCCCGCCGCGA
CCACCATCGCTTACGGCGTGATCGAGCTTTACGTGAAGCTGGACGGACAGTTCGAGTTCTGTCTGCTGCG
GGGGAAGCAGGGCGGTTTCGAAAACAAGAAGCGGATCGATAGCGTGTACCTGGACCCGCTGGTGTTCCGC
GAGTTCGCGTTTATTGACATGCCTGACGCCGCACACGGCATCTCGTCACAAGACGGGCCACTCAGCGTGC
TGAAGCAGGCCACCCTGCTGCTCGAGCGGAATTTCCATCCATTCGCTGAGCTCCCGGAACCCCAGCAGAC
TGCCCTGTCCGACATTTTCCAAGCCGTGCTGTTCGACGACGAACTGTTAATGGTGCTCGAGCCTGTGTGC
GATGATCTGGTGTCCGGCCTGTCCCCAACCGTGGCCGTCCTGGGCGAACTCAAGCCTAGGCAGCAGCAGG
ACCTGGTGGCGTTCCTTCAACTCGTCGGATGCTCGCTGCAAGGAGGCTGCCCTGGACCTGAGGATGCCGG
TTCCAAGCAGCTGTTCATGACGGCCTACTTCCTGGTGTCGGCCCTGGCCGAAATGCCGGACTCCGCAGCA
GCGCTTTTGGGCACCTGTTGCAAACTGCAGATCATTCCGACCCTGTGCCACCTTCTGCGCGCCCTGAGCG
ATGATGGAGTGTCAGACTTGGAGGACCCCACCCTCACTCCGCTGAAGGACACCGAACGGTTCGGCATCGT
GCAGAGACTGTTTGCTTCCGCGGATATCTCCCTGGAACGCCTGAAGTCCAGCGTCAAGGCCGTGATTCTG
AAGGACTCGAAGGTCTTTCCGCTGCTTTTGTGCATCACCCTCAACGGACTCTGCGCTCTCGGTCGCGAGC
ACAGCTAATGATAATAGGCTGGAGCCTCGGTGGCCATGCTTCTTGCCCCTTGGGCCTCCCCCCAGCCCCT
CCTCCCCTTCCTGCACCCGTACCCCCCAAACACCATTGTCACACTCCAGTGGTCTTTGAATAAAGTCTGA
GTGGGCGGCGCGGCCGCAAAAAAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAAAAAAAAAAAAAAAAAAAAAAAAAAA
AAAAAAA
Alternative 3′UTR
(SEQ ID NO: 68)
TGATAATAGTCCATAAAGTAGGAAACACTACAGCTGGAGCCTCGGTGGCCATGCTTCTTGCCCCTTGGGC
CCAAACACCATTGTCACACTCCATCCCCCCAGCCCCTCCTCCCCTTCCTGCACCCGTACCCCCGTGGTCT
TTGAATAAAGTCTGAGTGGGCGGCGCGGCCGCAAAAAAAAAAAAAAAAAAAAAAAAAAAAAAAAAAAAAA
AAAAAAAAAAAAAAAAAAAAAAAAAAAAAAAAAAAAAAAAAAAAAAAAAAAAAAAAAAAAAAAAAAAAAA
AAAAAAAAAAAAAAAAAAAAAA
5′ UTR-underlined
GSDME coding region-plain text1
3′ UTR-italic
miR122 (CAAACACCATTGTCACACTCCA)-bold, underlined, italic
miR142 (TCCATAAAGTAGGAAACACTACA)-bold, underlined, italic
polyA tail-bold
Mouse RIPK3 (mRIPK3) Construct DNA Sequence
SEQ ID NO: 41
GGGAAATAAGAGAGAAAAGAAGAGTAAGAAGAAATATAAGACCCCGGCGCCGCCACCATGTCGTCCGTCA
AGCTGTGGCCAACCGGAGCTAGTGCCGTGCCTCTTGTGTCCCGGGAGGAGCTCAAGAAGTTGGAGTTCGT
GGGAAAGGGAGGATTCGGCGTCGTGTTCCGGGCGCACCACAGAACTTGGAACCATGACGTGGCCGTGAAG
ATCGTGAACTCGAAGAAGATCTCCTGGGAAGTCAAGGCCATGGTCAACTTGCGCAACGAGAATGTGCTGC
TCCTGCTGGGCGTCACCGAGGATCTGCAGTGGGACTTTGTGTCCGGTCAAGCCCTCGTCACCCGCTTCAT
GGAGAACGGATCCCTGGCCGGGCTGCTTCAGCCGGAATGTCCGCGCCCGTGGCCGCTGCTGTGCCGGCTG
CTGCAAGAAGTGGTGCTGGGGATGTGCTATCTTCACTCGCTGAACCCGCCCTTGTTACACCGCGACCTGA
AGCCAAGCAACATTCTGCTGGATCCCGAGCTCCACGCCAAGCTGGCCGACTTCGGACTGTCAACGTTCCA
AGGCGGTAGCCAGTCGGGTTCCGGATCGGGATCAGGATCGAGGGATTCAGGAGGGACCCTGGCCTACCTG
GACCCGGAACTCCTGTTCGATGTGAACCTGAAGGCGTCCAAGGCCTCCGACGTGTACAGCTTTGGGATCC
TCGTCTGGGCAGTGCTGGCCGGTCGAGAAGCGGAGCTTGTGGACAAGACCAGCCTGATTAGGGAAACTGT
GTGCGATCGCCAGTCCCGGCCTCCTCTGACTGAACTCCCGCCCGGATCCCCGGAAACCCCAGGACTGGAG
AAGCTGAAGGAGCTGATGATCCACTGCTGGGGGTCCCAGAGCGAAAACAGACCATCATTCCAAGACTGCG
AACCTAAGACTAACGAAGTGTACAATCTCGTGAAAGATAAGGTCGACGCTGCCGTGTCCGAGGTCAAGCA
TTACCTAAGCCAGCACAGGTCGTCCGGCCGCAACCTCAGCGCGCGGGAGCCCAGCCAGAGAGGCACCGAG
ATGGACTGCCCGCGGGAAACCATGGTGTCCAAAATGCTGGACAGGTTGCACCTGGAAGAACCCTCAGGAC
CGGTGCCAGGAAAGTGTCCAGAGAGACAGGCCCAGGACACAAGCGTGGGCCCTGCAACCCCCGCTCGGAC
TTCCTCCGATCCGGTGGCCGGCACCCCGCAAATCCCTCACACCCTGCCGTTCCGCGGCACCACCCCCGGC
CCTGTGTTCACCGAAACTCCGGGGCCCCATCCCCAACGGAACCAGGGCGACGGGAGACACGGTACTCCTT
GGTACCCCTGGACCCCTCCTAACCCTATGACCGGTCCGCCCGCCCTCGTGTTCAACAACTGCTCCGAGGT
GCAGATTGGCAACTACAATTCGCTCGTGGCCCCGCCCCGGACTACCGCTTCCTCCTCTGCGAAATACGAC
CAGGCACAGTTTGGCCGCGGACGGGGATGGCAGCCCTTCCATAAG
TAATGATAATAGGCTGGAGCCTCGGTGGCCATGCTTCTTGCCCCTTGGGCCTCCCCCCAGCCCCTCCTCC
CCTTCCTGCACCCGTACCCCCCAAACACCATTGTCACACTCCAGTGGTCTTTGAATAAAGTCTGAGTGGG
CGGCGCGGCCGCAAAAAAAAAAAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAAAAAAAAAAAAAAAAAAAAAAAAAAA
AA
Alternative 3′UTR
(SEQ ID NO: 68)
TGATAATAGTCCATAAAGTAGGAAACACTACAGCTGGAGCCTCGGTGGCCATGCTTCTTGCCCCTTGGGC
CCAAACACCATTGTCACACTCCATCCCCCCAGCCCCTCCTCCCCTTCCTGCACCCGTACCCCCGTGGTCT
TTGAATAAAGTCTGAGTGGGCGGCGCGGCCGCAAAAAAAAAAAAAAAAAAAAAAAAAAAAAAAAAAAAAA
AAAAAAAAAAAAAAAAAAAAAAAAAAAAAAAAAAAAAAAAAAAAAAAAAAAAAAAAAAAAAAAAAAAAAA
AAAAAAAAAAAAAAAAAAAAAA
5′ UTR-underlined
mRIPK3 coding region-plain textCap1
3′ UTR-italic
miR122 (CAAACACCATTGTCACACTCCA)-bold and underlined
miR142 (TCCATAAAGTAGGAAACACTACA)-bold and underlined
polyA tail-bold
Human RIPK3 (hRIPK3) Construct DNA sequence
SEQ ID NO: 42
GGGAAATAAGAGAGAAAAGAAGAGTAAGAAGAAATATAAGACCCCGGCGCCGCCACCATGAGCTGTGTGAA
GCTGTGGCCTTCCGGCGCCCCAGCCCCTCTGGTGTCCATTGAGGAACTGGAAAACCAGGAGCTGGTCGGAAAGG
GCGGATTCGGCACAGTGTTCAGAGCCCAGCACCGGAAGTGGGGATACGACGTGGCCGTGAAAATCGTGAACAGC
AAGGCCATCAGCAGAGAGGTGAAAGCCATGGCCTCTCTGGATAATGAGTTCGTGCTGCGGCTGGAAGGAGTGAT
CGAAAAGGTGAATTGGGACCAGGACCCTAAGCCCGCACTGGTGACCAAGTTCATGGAAAACGGCAGCCTGAGCG
GCCTACTTCAGTCTCAATGTCCTAGACCCTGGCCTCTGCTGTGTAGACTGCTGAAAGAGGTGGTGCTGGGCATGT
TCTACCTGCATGATCAGAACCCCGTGCTGCTGCACAGAGATCTGAAGCCTTCTAATGTGCTGCTGGACCCCGAGC
TGCACGTGAAGCTCGCTGATTTCGGCCTGTCTACATTCCAGGGCGGCTCGCAGAGCGGCACTGGCAGCGGCGAAC
CTGGCGGGACCCTGGGATATCTGGCCCCTGAACTCTTCGTGAACGTGAACAGAAAGGCCAGCACAGCTAGCGACG
TGTACAGCTTCGGCATCCTGATGTGGGCCGTGCTGGCCGGCAGAGAAGTGGAACTGCCCACCGAGCCTAGCCTGG
TGTACGAGGCCGTGTGCAACCGCCAGAACCGGCCTAGCCTGGCTGAACTGCCTCAAGCTGGCCCGGAAACCCCTG
GCCTCGAAGGCCTGAAGGAACTGATGCAGCTGTGCTGGTCCTCTGAGCCGAAGGACAGACCTTCCTTTCAGGAGT
GCCTGCCTAAGACCGACGAGGTCTTTCAGATGGTGGAAAACAACATGAACGCCGCCGTCAGCACCGTGAAGGACT
TTCTGAGCCAACTGAGAAGCAGCAACAGAAGGTTCAGCATCCCCGAGTCCGGACAGGGCGGCACAGAGATGGACG
GCTTCAGACGGACCATCGAGAATCAGCACAGCAGAAACGACGTGATGGTCAGCGAGTGGCTGAACAAGCTGAAC
CTGGAGGAACCCCCTAGCAGCGTGCCCAAAAAGTGCCCTTCCCTGACAAAGCGGAGCCGGGCCCAGGAGGAACA
GGTTCCTCAGGCCTGGACCGCCGGCACCAGCTCTGACAGCATGGCCCAGCCTCCTCAAACACCAGAGACAAGTAC
CTTCCGGAACCAGATGCCTTCTCCCACCAGCACCGGCACCCCTAGCCCCGGCCCTAGAGGCAACCAGGGCGCCGA
ACGGCAGGGCATGAACTGGAGCTGCAGAACACCTGAGCCCAACCCTGTGACAGGCAGACCCCTGGTGAATATCTA
CAATTGCAGCGGCGTGCAGGTGGGAGATAACAACTACCTGACCATGCAGCAGACCACCGCCCTGCCAACCTGGGG
CCTGGCTCCATCTGGCAAAGGCCGGGGGCTGCAGCACCCCCCTCCTGTGGGCAGCCAGGAGGGACCTAAGGATC
CTGAGGCCTGGAGCAGACCACAGGGATGGTACAATCACAGCGGAAAA
TAATGATAATAGGCTGGAGCCTCGGTGGCCATGCTTCTTGCCCCTTGGGCCTCCCCCCAGCCCCTCCTCC
CCTTCCTGCACCCGTACCCCCCAAACACCATTGTCACACTCCAGTGGTCTTTGAATAAAGTCTGAGTGGG
CGGCGCGGCCGCAAAAAAAAAAAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAAAAAAAAAAAAAAAAAAAAAAAAAAA
AA
Alternative 3′UTR
(SEQ ID NO: 68)
TGATAATAGTCCATAAAGTAGGAAACACTACAGCTGGAGCCTCGGTGGCCATGCTTCTTGCCCCTTGGGC
CCAAACACCATTGTCACACTCCATCCCCCCAGCCCCTCCTCCCCTTCCTGCACCCGTACCCCCGTGGTCT
TTGAATAAAGTCTGAGTGGGCGGCGCGGCCGCAAAAAAAAAAAAAAAAAAAAAAAAAAAAAAAAAAAAAA
AAAAAAAAAAAAAAAAAAAAAAAAAAAAAAAAAAAAAAAAAAAAAAAAAAAAAAAAAAAAAAAAAAAAAA
AAAAAAAAAAAAAAAAAAAAAA
5′ UTR-underlined
hRIPK3 coding region-plain text
3′ UTR-italic
miR122 (CAAACACCATTGTCACACTCCA)-bold, underlined, italic
miR142 (TCCATAAAGTAGGAAACACTACA)-bold, underlined, italic
polyA tail-bold
VICA (UL36) Construct DNA Sequence
SEQ ID NO: 43
GGGAAATAAGAGAGAAAAGAAGAGTAAGAAGAAATATAAGACCCCGGCGCCGCCACCATGGATGATCTGC
GCGACACCTTGATGGCCTACGGCTGCATCGCCATTCGCGCCGGAGATTTCAACGGCCTGAATGACTTCCT
GGAGCAAGAGTGCGGAACCAGACTTCACGTGGCCTGGCCGGAACGGTGCTTCATCCAACTGCGGTCGCGC
TCAGCACTGGGACCTTTCGTGGGGAAGATGGGAACCGTGTGTAGCCAGGGTGCATACGTGTGCTGTCAGG
AGTATCTGCACCCATTCGGCTTTGTGGAAGGCCCCGGCTTCATGCGGTACCAGCTGATTGTGCTGATCGG
CCAGCGCGGCGGAATCTACTGCTACGATGACCTCAGAGACTGCATCTACGAACTGGCCCCTACTATGAAG
GACTTCCTGAGACACGGCTTCCGCCATTGCGATCATTTCCACACCATGCGCGACTATCAGCGGCCGATGG
TGCAGTACGACGACTACTGGAACGCTGTGATGCTCTACCGGGGAGATGTGGAGAGCCTGTCCGCCGAAGT
GACCAAGAGGGGTTACGCTAGCTACTCCATCGACGATCCCTTCGACGAATGTCCCGACACTCACTTCGCC
TTCTGGACCCATAACACCGAGGTCATGAAGTTTAAGGAAACCTCGTTCTCCGTGGTCCGGGCCGGAGGGT
CTATCCAGACCATGGAGCTGATGATTAGAACCGTGCCTCGGATCACGTGTTACCACCAACTTCTCGGCGC
CTTGGGTCACGAAGTGCCTGAACGGAAGGAGTTCCTCGTGCGGCAATACGTGCTGGTCGACACCTTCGGC
GTGGTGTACGGTTACGACCCTGCGATGGACGCGGTGTACCGCCTCGCCGAGGACGTCGTCATGTTCACTT
GCGTGATGGGGAAGAAGGGACATCGGAACCACCGCTTCTCGGGAAGGAGGGAAGCTATCGTCCGCCTGGA
AAAGACCCCGACGTGCCAGCACCCGAAGAAAACACCCGATCCAATGATCATGTTCGACGAGGATGACGAT
GACGAGCTGAGCCTGCCCAGAAACGTGATGACCCACGAAGAAGCCGAATCCCGGCTCTACGACGCAATTA
CCGAGAACCTCATGCATTGCGTGAAGCTTGTCACTACTGATTCCCCTCTGGCCACTCACTTGTGGCCGCA
AGAGCTGCAGGCCCTCTGCGACTCCCCGGCGCTGTCCCTGTGCACCGACGACGTGGAAGGAGTGCGCCAG
AAGCTGAGAGCCAGGACTGGATCCCTGCACCACTTTGAGCTGTCCTATCGATTCCACGACGAGGACCCGG
AAACCTACATGGGGTTCCTGTGGGACATTCCATCATGCGATAGATGCGTGCGCCGGCGGAGGTTTAAAGT
CTGTGACGTGGGACGCAGGCACATCATTCCCGGAGCGGCGAATGGGATGCCGCCTCTCACTCCCCCGCAT
GCCTACATGAACAACTAATGATAATAGGCTGGAGCCTCGGTGGCCATGCTTCTTGCCCCTTGGGCCTCCC
CCCAGCCCCTCCTCCCCTTCCTGCACCCGTACCCCCCAAACACCATTGTCACACTCCAGTGGTCTTTGAA
TAAAGTCTGAGTGGGCGGCGCGGCCGCAAAAAAAAAAAAAAAAAAAAAAAAAAAAAAAAAAAAAAAAAAA
AAAAAAAAAAAAAAAAAAAAAAAAAAAAAAAAAAAAAAAAAAAAAAAAAAAAAAAAAAAAAAAAAAAAAA
AAAAAAAAAAAAAAAAA
Alternative 3′UTR
(SEQ ID NO: 68)
TGATAATAGTCCATAAAGTAGGAAACACTACAGCTGGAGCCTCGGTGGCCATGCTTCTTGCCCCTTGGGC
CCAAACACCATTGTCACACTCCATCCCCCCAGCCCCTCCTCCCCTTCCTGCACCCGTACCCCCGTGGTCT
TTGAATAAAGTCTGAGTGGGCGGCGCGGCCGCAAAAAAAAAAAAAAAAAAAAAAAAAAAAAAAAAAAAAA
AAAAAAAAAAAAAAAAAAAAAAAAAAAAAAAAAAAAAAAAAAAAAAAAAAAAAAAAAAAAAAAAAAAAAA
AAAAAAAAAAAAAAAAAAAAAA
Cap1
5′ UTR-underlined
VICA coding region-plain text
3′ UTR-italic
miR122 (CAAACACCATTGTCACACTCCA)-bold, underlined, italic
miR142 (TCCATAAAGTAGGAAACACTACA)-bold, underlined, italic
polyA tail-bold
Human wildtype Gasdermin E
SEQ ID NO: 44
ATGTTCGCCAAGGCCACCAGAAACTTCCTGAGGGAAGTGGACGCCGACGGGGACTTGATTGCGGTGTCCAACCTC
AACGACTCCGACAAGCTCCAGCTGCTTAGCCTGGTCACCAAGAAGAAGCGGTTCTGGTGCTGGCAGCGGCCCAAG
TACCAGTTCCTGTCGCTGACACTAGGCGACGTCTTGATAGAGGACCAGTTCCCCTCCCCGGTGGTCGTGGAAT
CCGATTTCGTGAAATATGAGGGGAAGTTCGCCAATCACGTGTCCGGAACTCTGGAAACTGCCCTGGGAAA
GGTCAAGTTGAACCTGGGCGGAAGCTCTAGGGTGGAATCGCAGTCCTCCTTTGGTACCCTGCGCAAGCAG
GAAGTGGATCTGCAGCAGCTCATTCGGGATAGCGCCGAGCGCACCATCAACCTGAGAAACCCCGTGCTCC
AACAAGTGCTGGAAGGGAGAAACGAAGTGCTCTGTGTGCTGACTCAGAAGATCACTACGATGCAGAAATG
CGTGATCTCAGAGCATATGCAAGTCGAGGAGAAATGCGGAGGAATCGTAGGAATTCAGACCAAGACTGTC
CAAGTGTCCGCCACTGAGGACGGAAACGTGACCAAGGACTCAAACGTGGTGCTGGAAATCCCCGCCGCGA
CCACCATCGCTTACGGCGTGATCGAGCTTTACGTGAAGCTGGACGGACAGTTCGAGTTCTGTCTGCTGCG
GGGGAAGCAGGGCGGTTTCGAAAACAAGAAGCGGATCGATAGCGTGTACCTGGACCCGCTGGTGTTCCGC
GAGTTCGCGTTTATTGACATGCCTGACGCCGCACACGGCATCTCGTCACAAGACGGGCCACTCAGCGTGC
TGAAGCAGGCCACCCTGCTGCTCGAGCGGAATTTCCATCCATTCGCTGAGCTCCCGGAACCCCAGCAGAC
TGCCCTGTCCGACATTTTCCAAGCCGTGCTGTTCGACGACGAACTGTTAATGGTGCTCGAGCCTGTGTGC
GATGATCTGGTGTCCGGCCTGTCCCCAACCGTGGCCGTCCTGGGCGAACTCAAGCCTAGGCAGCAGCAGG
ACCTGGTGGCGTTCCTTCAACTCGTCGGATGCTCGCTGCAAGGAGGCTGCCCTGGACCTGAGGATGCCGG
TTCCAAGCAGCTGTTCATGACGGCCTACTTCCTGGTGTCGGCCCTGGCCGAAATGCCGGACTCCGCAGCA
GCGCTTTTGGGCACCTGTTGCAAACTGCAGATCATTCCGACCCTGTGCCACCTTCTGCGCGCCCTGAGCG
ATGATGGAGTGTCAGACTTGGAGGACCCCACCCTCACTCCGCTGAAGGACACCGAACGGTTCGGCATCGT
GCAGAGACTGTTTGCTTCCGCGGATATCTCCCTGGAACGCCTGAAGTCCAGCGTCAAGGCCGTGATTCTG
AAGGACTCGAAGGTCTTTCCGCTGCTTTTGTGCATCACCCTCAACGGACTCTGCGCTCTCGGTCGCGAGC
ACAGC
N-terminal protease (Npro) viral protein
SEQ ID NO: 45
ATGGAACTGAACCATTTCGAGCTGCTGTACAAGACAAGCAAGCAGAAGCCTGTGGGCGTGGAAGAGCCTGTGTAC
GACACCGCCGGCCGGCCTCTGTTCGGCAACCCCAGCGAAGTGCACCCCCAGTCTACACTTAAACTGCCACACGAT
AGAGGCCGGGGCGATATCAGAACAACACTGAGAGATCTGCCTAGAAAGGGCGACTGTAGAAGCGGCAATCACCTG
GGCCCTGTGTCCGGAATCTACATTAAGCCCGGCCCCGTGTACTACCAGGACTACACCGGCCCAGTTTATCACAGA
GCCCCTCTCGAGTTCTTCGACGAGGCTCAATTTTGCGAGGTGACCAAGAGAATCGGCAGAGTGACCGGAAGCGAC
GGCAAGCTGTACCACATCTACGTGTGCGTGGACGGATGTATCCTGCTGAAGCTGGCCAAGCGGGGAACCCCTCGC
ACCCTGAAATGGATCCGGAACTTCACCAACTGCCCTCTGTGGGTCACCAGCTGC
Human wildtype A238L
SEQ ID NO: 46
ATGGACACCATCGGCCTGTTCAGCGTTGAAGCCGAGCACCTGTTTGTGGAATGGGTCAAGAAGTGCATCAAGAAG
GGAGATCTGACCCTGTTCGAGACACTGTTTAACGCCGATCCTTGGATCGTGAATAGATGCAACAAGAACAAAATC
ACCGTGTTTATGCTGATCTGTATCTACGGAAGACTGGACTTCCTCCGCTTCCTGTTTAAGCAGGAGAGCTACCCC
GGCGAGATCGTGAACCACTATAGACGGGACAAGGACGGCAACTCCGCCTGGCATTACCTGGCCGAAAAGAACAAC
CACCTGCTGCTGGAAGAAGTGCTGGATTACTTCGGCAAGAATGGCATCAGGGTGTGCTTCCCTAATTTCAACGGC
GTGACCCCTATCATGAAGGCCGCTATGCGGGGCAGAACACTTTCTGTGCTGAGCCTGCTGAAATACGGAGCTAAT
CCTAACCGGAAGGACTACCTGAAGGGCTTCACAACTTGGGATTGGGCCGTGTTCACCGGCCACGCCGACCTGGTC
AAGACCCTGAACAAGGGCTACCAGAAGCCACTGTTCATGCACTTCCCCCTGTACAAGCTGGACGTGTTCCACAGA
AGATTCAAGAAAAAGCCCAAGATCATCATTACCGGCTGCGAGGACAACGTGTACGAGAAGCTGCCTGAGCAGAAC
AGCAACTTCCTGTGTGTGAAAAAACTGAACAAGTACGGCAAA
viral wildtype vMLKL
SEQ ID NO: 47
ATGACCGACCCCCTGCTGCACAAGCTCTTTCCAGTCGCCGTTAACTCCAACATCAAAGTGATCAAGGACACAGAT
GTGGTGAAGGTGGAAAATGAGAACATCTGCGAGTGCAAAAACTCTTATGTGTACAACGCCATCTACAACAACGAG
GAAGCCCTGGTGATCATGTTCAAACACAGCCATACAGGCTACAAGGTGCTGCTGGACATCAGCGAGAAGTACAAT
ATTCTGCAACAGAAGCACGAGAAGAACGTGCTGAGAATCTACGCCGTGTTCATCGACAACAGCTACATCAACTTC
AAGCTGCCTAAGATCTCTCTGATCGTGGAAAAGTGCAAGCCTATCAGACGGCTGATCCTGGAAAAGATCAACCTG
CCTTTCGAGAGCAAGATTGACCTGATCCTGAACGCTCTGGATAGCCTGATTCTCCTGTACACCTACACAAAGGAG
CCATACAAGTACATCAACTCCAGCAACTTCCTGATCAATTCTGATTACGACGTGAAAATCGTGTCTCACGGACTG
GAAATCATCCTGAGCAATCCTGCCTTTAACACCATCAAGAGCAGCTCCTACTACAGCCCCAAGATCATCATGAAC
CCCTTCAGCAAGCAGACCATCAGCGACGACATCTATAGCTTCGGCATCCTGATGTACGAGATCCTGAGCTACAAA
ATCCCCTTCGAAAACATGAAATCAAGAGATATTATCAGCTGCGTGGAGAGCAACAAGATCGAGAGCATCCTTAAT
ATCAGCTCTATCAACTGTCCTGACACCCTGAAGAACCTGATCAAGAAGTGTGTGTCCATCGATCCTGTCATCCGG
CCTTCTTTTAAGGAAATCTACCAGAACATCAGCATCTACAAGTTCAATAAGAAAATTGAG
VICA
SEQ ID NO: 48
ATGGACGATCTGAGAGATACACTGATGGCCTATGGATGTATCGCCATCAGAGCCGGCGATTTCAACGGCCTGAAC
GACTTCCTGGAGCAGGAGTGTGGAACAAGACTGCATGTGGCCTGGCCTGAGCGGTGCTTCATCCAGCTGCGGTCT
AGAAGCGCCCTCGGCCCTTTCGTGGGAAAAATGGGGACCGTGTGCAGCCAGGGCGCCTACGTGTGCTGCCAAGAA
TACCTGCACCCTTTTGGCTTCGTGGAAGGACCTGGCTTCATGCGGTACCAGCTTATCGTGCTGATCGGCCAGAGA
GGCGGCATCTACTGCTACGACGACCTGAGAGACTGCATCTACGAGCTGGCCCCTACAATGAAGGACTTTCTGAGA
CACGGCTTTAGACACTGTGATCACTTTCACACAATGAGAGATTACCAGCGGCCTATGGTGCAGTACGACGACTAC
TGGAATGCCGTGATGCTGTACCGGGGCGACGTGGAAAGCCTGTCCGCCGAGGTGACAAAGAGAGGCTACGCCTCT
TATAGCATCGACGACCCCTTCGACGAGTGCCCCGACACCCACTTCGCCTTCTGGACCCACAACACCGAGGTCATG
AAATTCAAGGAAACCTCCTTCAGCGTGGTCCGGGCTGGCGGAAGCATTCAGACCATGGAACTGATGATCAGGACC
GTGCCCAGAATCACATGCTACCACCAGCTGCTGGGCGCTCTGGGCCACGAGGTTCCAGAAAGAAAGGAGTTCCTG
GTCAGACAGTACGTGCTGGTGGACACCTTCGGCGTGGTGTACGGATATGACCCCGCTATGGATGCTGTGTACCGC
CTGGCTGAGGACGTGGTGATGTTCACCTGCGTGATGGGCAAGAAAGGCCACAGAAATCACCGGTTCAGCGGCAGA
CGGGAAGCCATCGTGCGGCTGGAAAAGACCCCTACCTGTCAACACCCTAAGAAGACACCTGATCCAATGATCATG
TTCGACGAGGATGATGACGATGAACTGAGCCTGCCTAGAAACGTGATGACCCACGAGGAAGCCGAGAGCAGACTG
TACGACGCCATTACCGAGAACCTGATGCACTGCGTTAAGCTGGTGACCACCGATAGCCCCCTGGCCACACATCTG
TGGCCCCAGGAGCTGCAGGCCCTGTGCGACAGCCCTGCCCTGTCCCTGTGTACAGATGACGTGGAGGGCGTTAGA
CAGAAGCTGCGGGCCAGAACCGGCAGCTTGCACCATTTCGAGCTGTCTTACAGATTCCACGACGAGGACCCTGAA
ACCTACATGGGATTCCTGTGGGACATCCCATCTTGTGATAGATGCGTGAGAAGAAGACGCTTTAAGGTGTGCGAC
GTGGGCAGAAGGCACATCATCCCCGGCGCCGCCAACGGCATGCCTCCACTGACCCCTCCTCACGCCTACATGAAC
AAC
IKBa dominant negative variant (IKBa_DN) ; S32A, S36A
SEQ ID NO: 49
ATGTTCCAGGCCGCTGAAAGACCTCAGGAGTGGGCCATGGAAGGCCCTAGGGATGGCCTGAAGAAGGAGCGGCTG
CTGGATGATAGACACGACGCCGGCCTGGACGCCATGAAAGACGAAGAGTACGAGCAGATGGTGAAGGAACTGCAG
GAGATCAGACTGGAACCACAAGAGGTGCCCAGAGGAAGCGAACCCTGGAAGCAGCAGCTGACCGAGGACGGCGAC
TCTTTCCTGCACCTGGCCATCATCCACGAGGAGAAGGCCCTGACCATGGAAGTGATCCGGCAGGTTAAGGGCGAT
CTGGCTTTTCTGAACTTCCAAAACAACCTGCAACAAACACCTCTGCACCTGGCTGTGATCACCAATCAGCCTGAG
ATCGCCGAGGCCCTGCTGGGAGCCGGCTGTGATCCTGAGCTGCGGGACTTCCGCGGCAACACCCCTCTGCATCTG
GCTTGTGAACAGGGCTGCCTGGCCTCTGTGGGCGTGCTGACACAGTCTTGCACCACACCCCACCTGCACAGCATC
CTGAAAGCCACAAATTACAACGGCCACACCTGCCTGCACTTGGCCAGCATCCACGGATACCTGGGTATTGTGGAA
CTGCTGGTGTCCCTGGGCGCTGATGTGAACGCCCAGGAGCCTTGCAACGGCAGAACAGCCCTGCACCTGGCCGTG
GACCTGCAGAACCCCGACCTGGTCAGCCTGCTACTCAAGTGCGGCGCCGACGTGAATAGAGTGACCTACCAGGGC
TACAGCCCTTATCAGCTGACCTGGGGCCGGCCCAGCACCAGAATCCAGCAGCAGCTTGGACAGCTGACTCTGGAA
AACCTGCAGATGCTCCCAGAGTCCGAAGATGAGGAAAGCTACGACACCGAGAGCGAGTTCACCGAGTTTACCGAG
GACGAGCTGCCTTACGACGACTGCGTGTTCGGCGGACAGAGACTGACACTG
IKBa human wildtype
SEQ ID NO: 50
MFQAAERPQEWAMEGPRDGLKKERLLDDRHDSGLDSMKDEEYEQMVKELQEIRLEPQEVP
RGSEPWKQQLTEDGDSFLHLAIIHEEKALTMEVI ROVKGDLAFLNFONNLQQTPLHLAVI
TNQPEIAEALLGAGCDPELRDFRGNTPLHLACEQGCLASVGVLTQSCTTPHLHSILKATN
YNGHTCLHLASIHGYLGIVELLVSLGADVNAQEPCNGRTALHLAVDLONPDLVSLLLKCG
ADVNRVTYQGYS PYQLTWGRPSTRIQQQLGOLTLENLOMLPESEDEESYDTESEFTEFTE
DELPYDDCVEGGQRLTL
Caspase 1 human wildtype
SEQ ID NO: 51
ATGGGCGTCCAGGTGGAAACCATCAGCCCTGGCGACGGCAGAACATTTCCAAAGCGGGGCCAGACTTGTGTGGTG
CACTACACCGGCATGCTGGAAGATGGAAAAAAGGTGGACTCCAGCAGAGATAGAAACAAACCTTTTAAGTTCATG
CTGGGCAAGCAGGAGGTGATCCGGGGATGGGAGGAAGGCGTGGCCCAGATGAGCGTGGGCCAGCGGGCCAAGCTC
ACAATCAGCCCAGACTACGCCTACGGCGCCACCGGACACCCTGGCATCATCCCCCCCCACGCCACACTGGTGTTT
GACGTTGAGCTGCTGAAGCTGGAAGGCTCTGGAAGCCCCGCTGCTGACAAAGTGCTGAAAGAGAAGCGCAAGCTG
TTTATAAGGTCCATGGGCGAGGGCACAATCAACGGCCTGCTGGACGAGCTGCTGCAGACCAGAGTGCTGAACAAG
GAAGAGATGGAAAAAGTGAAGAGAGAGAACGCCACCGTGATGGACAAGACCAGGGCCCTTATCGACAGCGTGATC
CCTAAGGGCGCACAAGCCTGTCAGATCTGTATCACATACATCTGCGAGGAGGATTCTTATCTGGCCGGCACCCTG
GGACTGTCTGCCGATCAGACAAGCGGCAATTACCTGAACATGCAGGACAGCCAGGGCGTGCTGTCTTCTTTCCCT
GCCCCTCAGGCCGTGCAGGATAATCCTGCCATGCCTACCAGCTCTGGATCTGAGGGAAACGTGAAGTTGTGCAGC
CTGGAAGAGGCCCAAAGAATCTGGAAGCAAAAGTCCGCCGAGATCTACCCCATCATGGACAAGAGCAGCAGAACC
AGACTGGCCCTGATCATCTGCAACGAGGAGTTCGACAGCATCCCCAGAAGAACCGGCGCTGAAGTGGATATCACC
GGCATGACCATGCTGCTGCAGAACCTGGGCTACAGCGTGGACGTGAAAAAGAACCTGACCGCCAGCGACATGACA
ACCGAGCTGGAAGCCTTCGCCCATAGACCTGAGCACAAGACCTCTGACTCTACATTCCTGGTGTTCATGAGCCAC
GGTATCCGGGAAGGCATCTGCGGCAAGAAGCACAGCGAGCAGGTTCCTGACATCCTGCAACTGAATGCCATCTTC
AACATGCTCAACACCAAGAACTGCCCCAGCCTGAAGGATAAGCCTAAGGTGATCATCATTCAGGCTTGTAGAGGC
GATAGCCCAGGCGTCGTGTGGTTCAAGGACAGCGTCGGCGTGTCCGGCAATCTGAGCCTGCCTACAACCGAAGAA
TTCGAGGACGACGCCATCAAGAAAGCCCACATCGAGAAAGACTTCATCGCTTTTTGCAGCAGCACCCCTGATAAC
GTGTCCTGGCGGCACCCCACAATGGGCAGCGTGTTCATTGGCAGACTGATCGAGCACATGCAGGAGTACGCCTGC
TCATGCGACGTGGAAGAAATCTTCCGGAAGGTGCGGTTCAGCTTCGAGCAGCCTGACGGAAGAGCGCAGATGCCT
ACCACCGAGAGAGTGACCCTGACACGGTGCTTCTACCTGTTCCCCGGCCAC
Caspase 1 human wildtype
SEQ ID NO: 52
MGVQVETISPGDGRTFPKRGQTCVVHYTGMLEDGKKVDSSRDRNKPFKFMLGKQEVIRGW
EEGVAQMSVGQRAKLTISPDYAYGATGHPGIIPPHATLVEDVELLKLEGSGSPAADKVLK
EKRKLFIRSMGEGTINGLLDELLQTRVLNKEEMEKVKRENATVMDKTRALIDSVIPKGAQ
ACQICITYICEEDSYLAGTLGLSADQTSGNYLNMQDSQGVLSSFPAPQAVQDNPAMPTSS
GSEGNVKLCSLEEAQRIWKQKSAEIYPIMDKSSRTRLALIICNEEFDSIPRRTGAEVDIT
GMTMLLQNLGYSVDVKKNLTASDMTTELEAFAHRPEHKTSDSTFLVEMSHGIREGICGKK
HSEQVPDILQLNAIFNMLNTKNCPSLKDKPKVIIIQACRGDSPGVVWFKDSVGVSGNLSL
PTTEEFEDDAIKKAHIEKDFIAFCSSTPDNVSWRHPTMGSVFIGRLIEHMQEYACSCDVE
EIFRKVRFSFEQPDGRAQMPTTERVTLTRCFYLFPGH
Caspase 1 human wildtype fragment in Figure 23 (Protein 1)
SEQ ID NO: 53
ADKVLKEKRKLFIRSMGEGTINGLLDELLQTRVLNKEEMEKVKRENATVMDKTRALIDSVIPKGAQ
ACQICITYICEEDSYLAGTLGLSADQTSGNYLNMODS
Caspase 1 variant with C-terminal self-dimerization domain (Casp1FV)
SEQ ID NO: 54
ADKVLKEKRKLFIRSMGEGTINGLLDELLQTRVLNKEEMEKVKRENATVMDKTRALIDSVIPKGAQ
ACQICITYICEEDSYLAGTLGLSADLSNNGQVKPHTS
Caspase 1 variant with C-terminal self-dimerization domain (Casp1FV)
SEQ ID NO: 55
GCTGACAAAGTGCTGAAAGAGAAGCGCAAGCTGTTTATAAGGTCCATGGGCGAGGGCACAATCAACGGCCTGCTG
GACGAGCTGCTGCAGACCAGAGTGCTGAACAAGGAAGAGATGGAAAAAGTGAAGAGAGAGAACGCCACCGTGATG
GACAAGACCAGGGCCCTTATCGACAGCGTGATCCCTAAGGGCGCACAAGCCTGTCAGATCTGTATCACATACATC
TGCGAGGAGGATTCTTATCTGGCCGGCACCCTGGGACTGTCTGCCGATTTATCTAATAATGGTCAAGTTAAACCT
CATACTTCT
Mouse IL-12 p40 subunit nucleic acid sequence
SEQ ID NO: 56
ATGTGCCCTCAAAAGCTCACCATTTCCTGGTTCGCCATCGTGCTTCTGGTGTCCCCGCTGATGGCTATGTGGGAG
CTTGAAAAGGATGTGTATGTGGTGGAAGTGGACTGGACCCCCGACGCGCCTGGAGAAACCGTGAACCTGACTTGC
GACACCCCTGAGGAGGACGACATCACATGGACTAGCGACCAGAGACACGGAGTGATTGGGAGTGGAAAGACCCTC
ACCATCACCGTGAAGGAGTTCCTGGATGCCGGACAGTACACTTGTCACAAGGGGGGAGAGACTCTGAGCCATTCG
CATCTGCTGCTCCACAAGAAGGAGAACGGCATCTGGTCCACTGAAATCCTGAAGAACTTCAAGAACAAGACCTTC
CTTAAGTGCGAGGCGCCCAACTACTCGGGAAGGTTCACGTGCTCATGGCTGGTCCAGAGAAACATGGACCTTAAG
TTCAACATCAAGAGCTCCTCGAGCTCCCCCGACTCTCGCGCTGTGACTTGTGGCATGGCTTCCTTGTCCGCCGAG
AAGGTCACCCTGGATCAGCGGGACTACGAAAAGTACAGCGTGTCGTGCCAAGAAGATGTGACCTGTCCTACCGCC
GAGGAAACCCTGCCGATCGAACTGGCCCTGGAAGCGCGGCAGCAGAATAAGTACGAAAACTACTCCACCTCGTTC
TTTATCCGGGACATTATCAAGCCCGATCCCCCGAAGAACCTCCAGATGAAGCCGCTGAAGAACAGCCAAGTGGAG
GTGTCATGGGAGTATCCAGACTCCTGGTCCACCCCGCACTCCTACTTCTCCCTGAAATTCTTCGTGCGGATCCAG
CGGAAGAAGGAAAAGATGAAGGAGACTGAAGAAGGCTGCAATCAGAAGGGGGCCTTTCTGGTGGAGAAAACTTCC
ACCGAGGTCCAGTGCAAAGGCGGAAACGTCTGTGTGCAAGCACAGGACCGATACTACAACTCCTCTTGCTCCAAA
TGGGCGTGCGTGCCTTGCCGCGTGCGGTCC
Mouse IL-12 p35 subunit nucleic acid sequence
SEQ ID NO: 57
CGCGTGATCCCTGTGTCCGGCCCCGCCCGCTGCCTGAGCCAGTCGAGGAACCTACTGAAAACCACCGACGACATG
GTCAAGACCGCCAGAGAAAAGCTGAAGCATTACTCCTGCACCGCGGAGGACATCGACCACGAGGACATTACGCGC
GATCAGACCTCAACGCTCAAAACTTGCTTGCCGCTCGAGCTGCACAAGAACGAAAGCTGCCTGGCCACTAGAGAA
ACTAGCTCCACTACCCGGGGTTCGTGTCTGCCGCCCCAAAAGACCTCACTCATGATGACCCTCTGCCTGGGCTCG
ATCTACGAGGATCTGAAGATGTACCAGACAGAGTTCCAGGCCATCAACGCCGCACTGCAAAACCACAATCACCAG
CAGATCATTTTGGACAAGGGCATGCTCGTGGCCATTGACGAATTGATGCAGTCCCTCAACCATAACGGCGAAACT
CTGAGGCAGAAGCCACCGGTCGGCGAAGCAGATCCATACCGCGTCAAGATGAAGCTCTGCATCCTGCTGCACGCC
TTTTCAACCCGGGTGGTCACCATTAACCGCGTGATGGGTTACCTGAGCTCCGCC
G4S3 linker nucleic acid sequence
SEQ ID NO: 58
GGAGGAGGAGGGAGCGGTGGCGGAGGGTCGGGTGGAGGAGGTTCC
G4S3 linker
SEQ ID NO: 59
GGGGSGGGGSGGGGS
Human IL12A (p35) amino acid sequence (GenBank: AF101062.1)
SEQ ID NO: 60
MWPPGSASQPPPSPAAATGLHPAARPVSLQCRLSMCPARSLLLVATLVLLDHLSLARNLPVATPDPGMFPCLHHS
QNLLRAVSNMLQKARQTLEFYPCTSEEIDHEDITKDKTSTVEACLPLELTKNESCLNSRETSFITNGSCLASRKT
SEMMALCLSSIYEDLKMYQVEFKTMNAKLLMDPKRQIFLDQNMLAVIDELMQALNENSETVPQKSSLEEPDFYKT
KIKLCILLHAFRIRAVTIDRVMSYLNAS
Human IL12A (p35) nucleic acid sequence
SEQ ID NO: 61
atgtggccccctgggtcagcctcccagccaccgccctcacctgccgcggccacaggtctgcatccagcggctcgc
cctgtgtccctgcagtgccggctcagcatgtgtccagcgcgcagcctcctccttgtggctaccctggtcctcctg
gaccacctcagtttggccagaaacctccccgtggccactccagacccaggaatgttcccatgccttcaccactcc
caaaacctgctgagggccgtcagcaacatgctccagaaggccagacaaactctagaattttacccttgcacttct
gaagagattgatcatgaagatatcacaaaagataaaaccagcacagtggaggcctgtttaccattggaattaacc
aagaatgagagttgcctaaattccagagagacctctttcataactaatgggagttgcctggcctccagaaagacc
tcttttatgatggccctgtgccttagtagtatttatgaagacttgaagatgtaccaggtggagttcaagaccatg
aatgcaaagcttctgatggatcctaagaggcagatctttctagatcaaaacatgctggcagttattgatgagctg
atgcaggccctgaatttcaacagtgagactgtgccacaaaaatcctcccttgaagaaccggatttttataaaact
aaaatcaagctctgcatacttcttcatgctttcagaattcgggcagtgactattgatagagtgatgagctatctg
aatgcttcctaa
Human IL12B (p40) amino acid sequence (GenBank: AF180563.1)
SEQ ID NO: 62
MCHQQLVISWFSLVFLASPLVAIWELKKDVYVVELDWYPDAPGEMVVLTCDTPEEDGITWTLDQSSEVLGSGKTL
TIQVKEFGDAGQYTCHKGGEVLSHSLLLLHKKEDGIWSTDILKDQKEPKNKTFLRCEAKNYSGRFTCWWLTTIST
DLTFSVKSSRGSSDPQGVTCGAATLSAERVRGDNKEYEYSVECQEDSACPAAEESLPIEVMVDAVHKLKYENYTS
SFFIRDIIKPDPPKNLQLKPLKNSRQVEVSWEYPDTWSTPHSYFSLTFCVQVQGKSKREKKDRVFTDKTSATVIC
RKNASISVRAQDRYYSSSWSEWASVPCS
Human IL12B (p40) nucleic acid sequence
SEQ ID NO: 63
agcaagatgtgtcaccagcagttggtcatctcttggttttccctggtttttctggcatctcccctcgtggccata
tgggaactgaagaaagatgtttatgtcgtagaattggattggtatccggatgcccctggagaaatggtggtcctc
acctgtgacacccctgaagaagatggtatcacctggaccttggaccagagcagtgaggtcttaggctctggcaaa
accctgaccatccaagtcaaagagtttggagatgctggccagtacacctgtcacaaaggaggcgaggttctaagc
cattcgctcctgctgcttcacaaaaaggaagatggaatttggtccactgatattttaaaggaccagaaagaaccc
aaaaataagacctttctaagatgcgaggccaagaattattctggacgtttcacctgctggtggctgacgacaatc
agtactgatttgacattcagtgtcaaaagcagcagaggctcttctgacccccaaggggtgacgtgcggagctgct
acactctctgcagagagagtcagaggggacaacaaggagtatgagtactcagtggagtgccaggaggacagtgcc
tgcccagctgctgaggagagtctgcccattgaggtcatggtggatgccgttcacaagctcaagtatgaaaactac
accagcagcttcttcatcagggacatcatcaaacctgacccacccaagaacttgcagctgaagccattaaagaat
tctcggcaggtggaggtcagctgggagtaccctgacacctggagtactccacattcctacttctccctgacattc
tgcgttcaggtccagggcaagagcaagagagaaaagaaagatagagtcttcacggacaagacctcagccacggtc
atctgccgcaaaaatgccagcattagcgtgcgggcccaggaccgctactatagctcatcttggagcgaatgggca
tctgtgccctgcagttaggttctgatccagga
mIL-12 mRNA construct from Example 15
SEQ ID NO: 64
GGGAAATAAGAGAGAAAAGAAGAGTAAGAAGAAATATAAGACCCCGGCGCCGCCACC (5′ UTR)
ATGTGCCCTCAAAAGCTCACCATTTCCTGGTTCGCCATCGTGCTTCTGGTGTCCCCGCTGATGGCTATGTGGGAG
CTTGAAAAGGATGTGTATGTGGTGGAAGTGGACTGGACCCCCGACGCGCCTGGAGAAACCGTGAACCTGACTTGC
GACACCCCTGAGGAGGACGACATCACATGGACTAGCGACCAGAGACACGGAGTGATTGGGAGTGGAAAGACCCTC
ACCATCACCGTGAAGGAGTTCCTGGATGCCGGACAGTACACTTGTCACAAGGGGGGAGAGACTCTGAGCCATTCG
CATCTGCTGCTCCACAAGAAGGAGAACGGCATCTGGTCCACTGAAATCCTGAAGAACTTCAAGAACAAGACCTTC
CTTAAGTGCGAGGCGCCCAACTACTCGGGAAGGTTCACGTGCTCATGGCTGGTCCAGAGAAACATGGACCTTAAG
TTCAACATCAAGAGCTCCTCGAGCTCCCCCGACTCTCGCGCTGTGACTTGTGGCATGGCTTCCTTGTCCGCCGAG
AAGGTCACCCTGGATCAGCGGGACTACGAAAAGTACAGCGTGTCGTGCCAAGAAGATGTGACCTGTCCTACCGCC
GAGGAAACCCTGCCGATCGAACTGGCCCTGGAAGCGCGGCAGCAGAATAAGTACGAAAACTACTCCACCTCGTTC
TTTATCCGGGACATTATCAAGCCCGATCCCCCGAAGAACCTCCAGATGAAGCCGCTGAAGAACAGCCAAGTGGAG
GTGTCATGGGAGTATCCAGACTCCTGGTCCACCCCGCACTCCTACTTCTCCCTGAAATTCTTCGTGCGGATCCAG
CGGAAGAAGGAAAAGATGAAGGAGACTGAAGAAGGCTGCAATCAGAAGGGGGCCTTTCTGGTGGAGAAAACTTCC
ACCGAGGTCCAGTGCAAAGGCGGAAACGTCTGTGTGCAAGCACAGGACCGATACTACAACTCCTCTTGCTCCAAA
TGGGCGTGCGTGCCTTGCCGCGTGCGGTCC (Mouse IL-12 p40 subunit)
GGAGGAGGAGGGAGCGGTGGCGGAGGGTCGGGTGGAGGAGGTTCC (GGGGSGGGGSGGGGS linker)
CGCGTGATCCCTGTGTCCGGCCCCGCCCGCTGCCTGAGCCAGTCGAGGAACCTACTGAAAACCACCGACGACATG
GTCAAGACCGCCAGAGAAAAGCTGAAGCATTACTCCTGCACCGCGGAGGACATCGACCACGAGGACATTACGCGC
GATCAGACCTCAACGCTCAAAACTTGCTTGCCGCTCGAGCTGCACAAGAACGAAAGCTGCCTGGCCACTAGAGAA
ACTAGCTCCACTACCCGGGGTTCGTGTCTGCCGCCCCAAAAGACCTCACTCATGATGACCCTCTGCCTGGGCTCG
ATCTACGAGGATCTGAAGATGTACCAGACAGAGTTCCAGGCCATCAACGCCGCACTGCAAAACCACAATCACCAG
CAGATCATTTTGGACAAGGGCATGCTCGTGGCCATTGACGAATTGATGCAGTCCCTCAACCATAACGGCGAAACT
CTGAGGCAGAAGCCACCGGTCGGCGAAGCAGATCCATACCGCGTCAAGATGAAGCTCTGCATCCTGCTGCACGCC
TTTTCAACCCGGGTGGTCACCATTAACCGCGTGATGGGTTACCTGAGCTCCGCC (Mouse IL-12 p35
subunit)
TAATGATAATAGGCTGGAGCCTCGGTGGCCATGCTTCTTGCCCCTTGGGCCTCCCCCCAGCCCCTCCTCCCCTTC
CTGCACCCGTACCCCCCAAACACCATTGTCACACTCCAGTGGTCTTTGAATAAAGTCTGAGTGGGCGGCGCGGCC
GCAAAAAAAAAAAAAAAAAAAAAAAAAAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAAAAAAAAAAAAAA
3′ UTR-italic
miR122 binding site (CAAACACCATTGTCACACTCCA)-bold and underlined
polyA tail-bold
hTRIF_mRNA construct from Example 15
SEQ ID NO: 65
GGGAAATAAGAGAGAAAAGAAGAGTAAGAAGAAATATAAGACCCCGGCGCCGCCACC (5′ UTR)
ATGGCCTGTACCGGCCCCTCCCTCCCCTCGGCCTTCGACATCCTGGGAGCAGCGGGCCAAGACAAGCTCCTGTAC
CTGAAGCACAAGCTGAAAACTCCTAGGCCCGGATGTCAGGGCCAGGATCTGCTGCACGCGATGGTGCTGCTCAAG
CTGGGTCAAGAAACCGAGGCCCGCATCAGCCTGGAAGCCTTGAAAGCGGACGCCGTGGCCAGGCTCGTGGCCCGG
CAGTGGGCCGGAGTGGACTCCACTGAGGATCCGGAAGAACCCCCGGATGTGTCGTGGGCGGTGGCTCGCCTGTAC
CACCTCCTGGCTGAAGAGAAGTTATGCCCAGCCTCCCTCCGCGACGTGGCATACCAGGAGGCCGTGCGGACCCTC
TCCTCGCGGGACGACCATCGCCTCGGTGAACTGCAGGATGAAGCTCGCAACAGATGTGGCTGGGATATTGCTGGC
GATCCGGGATCCATTCGGACACTGCAGAGCAACCTTGGATGCCTGCCCCCATCGTCCGCGCTGCCGAGCGGCACC
CGATCCCTGCCACGCCCGATTGACGGAGTCTCCGACTGGTCACAGGGATGCAGCCTCCGGTCCACCGGATCCCCG
GCCTCACTCGCAAGCAACCTGGAAATCTCGCAGTCCCCAACTATGCCGTTCCTGTCACTGCATCGCTCCCCTCAT
GGACCATCCAAGCTGTGCGACGATCCACAGGCCTCCCTCGTGCCTGAACCTGTGCCGGGAGGCTGCCAGGAACCG
GAGGAGATGTCCTGGCCGCCTAGCGGGGAGATCGCCTCACCGCCTGAACTCCCGTCCTCCCCCCCTCCGGGTTTG
CCCGAAGTGGCCCCTGACGCGACCAGCACCGGACTGCCCGACACTCCCGCCGCGCCCGAAACCTCCACTAATTAC
CCGGTGGAATGCACCGAGGGGTCGGCAGGGCCACAGTCCCTGCCTCTGCCTATTCTGGAGCCTGTGAAGAATCCC
TGCTCTGTGAAGGACCAGACTCCTCTGCAACTGTCAGTGGAGGATACCACCTCGCCCAACACGAAGCCCTGCCCT
CCTACTCCAACTACCCCTGAGACTTCGCCTCCGCCGCCTCCCCCGCCACCCTCCTCCACCCCGTGTTCCGCCCAC
CTCACACCTTCCTCCCTGTTTCCGTCATCCCTGGAAAGTTCGTCGGAACAGAAATTCTACAACTTCGTGATCCTC
CACGCGCGGGCTGATGAGCATATCGCCTTGCGCGTGCGGGAGAAGTTGGAAGCACTGGGGGTGCCCGACGGTGCC
ACCTTCTGCGAGGATTTTCAGGTCCCCGGAAGGGGAGAGCTCAGCTGCCTGCAAGACGCCATCGACCACAGCGCC
TTCATCATCCTCCTTCTGACCTCAAACTTCGACTGCCGCCTGAGCCTGCACCAGGTCAACCAGGCCATGATGTCC
AACCTGACCAGACAGGGCTCACCCGACTGTGTGATTCCGTTCCTGCCACTGGAGAGCAGCCCCGCCCAGCTGTCC
TCCGACACCGCCTCACTACTGTCCGGCCTTGTCCGGCTGGATGAACACAGCCAGATCTTCGCAAGAAAGGTCGCG
AACACCTTCAAGCCGCACAGACTGCAGGCCCGGAAGGCCATGTGGAGAAAGGAGCAGGACACGAGAGCCCTTCGG
GAGCAGTCCCAGCATCTTGACGGGGAACGCATGCAGGCCGCTGCCTTGAACGCGGCGTACAGCGCCTATCTGCAA
TCGTACCTGTCCTACCAAGCACAGATGGAGCAGCTCCAAGTGGCTTTCGGAAGCCACATGAGCTTTGGAACTGGT
GCACCGTACGGCGCCAGGATGCCCTTCGGCGGACAAGTCCCTCTGGGTGCCCCCCCACCGTTCCCAACCTGGCCC
GGGTGCCCTCAGCCGCCCCCGCTTCACGCTTGGCAGGCCGGCACTCCTCCTCCCCCGTCCCCTCAACCGGCCGCT
TTCCCGCAATCCCTTCCCTTCCCACAATCTCCCGCCTTCCCGACTGCCTCCCCGGCCCCGCCTCAATCCCCGGGA
CTCCAGCCTTTGATCATTCACCACGCGCAAATGGTCCAGCTGGGGCTGAACAACCATATGTGGAACCAGCGGGGC
TCGCAGGCCCCCGAGGACAAGACCCAGGAAGCAGAG (hTRIF_)
TGATAATAGTCCATAAAGTAGGAAACACTACAGCTGGAGCCTCGGTGGCCATGCTTCTTGCCCCTTGGGCCCAAA
CACCATTGTCACACTCCATCCCCCCAGCCCCTCCTCCCCTTCCTGCACCCGTACCCCCGTGGTCTTTGAATAAAG
TCTGAGTGGGCGGCGCGGCCGCAAAAAAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAAAAAAAAAAAAAAAAAAAAAAAAAAAAAAAA
AA
3′ UTR-italic
miR122 binding site (CAAACACCATTGTCACACTCCA)-bold and underlined
miR142 binding site (TCCATAAAGTAGGAAACACTACA)-bold and underlined
polyA tail-bold
hGSDME mRNA construct from Example 15
SEQ ID NO: 66
GGGAAATAAGAGAGAAAAGAAGAGTAAGAAGAAATATAAGACCCCGGCGCCGCCACC (5′ UTR)
ATGTTCGCCAAGGCCACCAGAAACTTCCTGAGGGAAGTGGACGCCGACGGGGACTTGATTGCGGTGTCCAACCTC
AACGACTCCGACAAGCTCCAGCTGCTTAGCCTGGTCACCAAGAAGAAGCGGTTCTGGTGCTGGCAGCGGCCCAAG
TACCAGTTCCTGTCGCTGACACTAGGCGACGTCTTGATAGAGGACCAGTTCCCCTCCCCGGTGGTCGTGGAATCC
GATTTCGTGAAATATGAGGGGAAGTTCGCCAATCACGTGTCCGGAACTCTGGAAACTGCCCTGGGAAAGGTCAAG
TTGAACCTGGGCGGAAGCTCTAGGGTGGAATCGCAGTCCTCCTTTGGTACCCTGCGCAAGCAGGAAGTGGATCTG
CAGCAGCTCATTCGGGATAGCGCCGAGCGCACCATCAACCTGAGAAACCCCGTGCTCCAACAAGTGCTGGAAGGG
AGAAACGAAGTGCTCTGTGTGCTGACTCAGAAGATCACTACGATGCAGAAATGCGTGATCTCAGAGCATATGCAA
GTCGAGGAGAAATGCGGAGGAATCGTAGGAATTCAGACCAAGACTGTCCAAGTGTCCGCCACTGAGGACGGAAAC
GTGACCAAGGACTCAAACGTGGTGCTGGAAATCCCCGCCGCGACCACCATCGCTTACGGCGTGATCGAGCTTTAC
GTGAAGCTGGACGGACAGTTCGAGTTCTGTCTGCTGCGGGGGAAGCAGGGCGGTTTCGAAAACAAGAAGCGGATC
GATAGCGTGTACCTGGACCCGCTGGTGTTCCGCGAGTTCGCGTTTATTGACATGCCTGACGCCGCACACGGCATC
TCGTCACAAGACGGGCCACTCAGCGTGCTGAAGCAGGCCACCCTGCTGCTCGAGCGGAATTTCCATCCATTCGCT
GAGCTCCCGGAACCCCAGCAGACTGCCCTGTCCGACATTTTCCAAGCCGTGCTGTTCGACGACGAACTGTTAATG
GTGCTCGAGCCTGTGTGCGATGATCTGGTGTCCGGCCTGTCCCCAACCGTGGCCGTCCTGGGCGAACTCAAGCCT
AGGCAGCAGCAGGACCTGGTGGCGTTCCTTCAACTCGTCGGATGCTCGCTGCAAGGAGGCTGCCCTGGACCTGAG
GATGCCGGTTCCAAGCAGCTGTTCATGACGGCCTACTTCCTGGTGTCGGCCCTGGCCGAAATGCCGGACTCCGCA
GCAGCGCTTTTGGGCACCTGTTGCAAACTGCAGATCATTCCGACCCTGTGCCACCTTCTGCGCGCCCTGAGCGAT
GATGGAGTGTCAGACTTGGAGGACCCCACCCTCACTCCGCTGAAGGACACCGAACGGTTCGGCATCGTGCAGAGA
CTGTTTGCTTCCGCGGATATCTCCCTGGAACGCCTGAAGTCCAGCGTCAAGGCCGTGATTCTGAAGGACTCGAAG
GTCTTTCCGCTGCTTTTGTGCATCACCCTCAACGGACTCTGCGCTCTCGGTCGCGAGCACAGC (hGSDME)
TGATAATAGTCCATAAAGTAGGAAACACTACAGCTGGAGCCTCGGTGGCCATGCTTCTTGCCCCTTGGGCCCAAA
CACCATTGTCACACTCCATCCCCCCAGCCCCTCCTCCCCTTCCTGCACCCGTACCCCCGTGGTCTTTGAATAAAG
TCTGAGTGGGCGGCGCGGCCGCAAAAAAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAAAAAAAAAAAAAAAAAAAAAAAAAAAAAAAA
AA
3′ UTR-italic
miR122 (CAAACACCATTGTCACACTCCA)-bold and underlined
miR142 (TCCATAAAGTAGGAAACACTACA)-bold and underlined
polyA tail-bold
GGGGS linker
SEQ ID NO: 67
GGGGS

1. A recombinant nucleic acid molecule comprising:
a) a first polynucleotide encoding TRIF or a variant thereof; and
b) a second polynucleotide encoding an additional polypeptide selected from the group consisting of Gasdermin E, RIPK3, vICA, Npro, A238L, vMLKL, and variants thereof, and a dominant negative variant of IKBa.

2. (canceled)

3. The recombinant nucleic acid molecule of claim 1, wherein the second polynucleotide encodes Gasdermin E or a variant thereof.

4-12. (canceled)

13. A recombinant nucleic acid molecule comprising:
a) a first polynucleotide encoding IL-12 or a variant thereof; and
b) a second polynucleotide encoding an additional polypeptide selected from the group consisting of TRIF, RIPK3, Gasdermin E, vICA, Npro, A238L, vMLKL, and variants thereof, and a dominant negative variant of IKBa.

14-16. (canceled)

17. The recombinant nucleic acid molecule of claim 13, wherein the second polynucleotide encodes TRIF or a variant thereof, and the recombinant nucleic acid molecule further comprises a third polynucleotide encoding Gasdermin E or a variant thereof.

18-28. (canceled)

29. The recombinant nucleic acid molecule of claim 1, wherein the nucleic acid molecule is a DNA molecule.

30. The recombinant nucleic acid molecule of claim 1, wherein the nucleic acid molecule is an RNA molecule.

31. The recombinant nucleic acid molecule of claim 30, wherein the RNA molecule is an mRNA molecule.

32. (canceled)

33. The recombinant nucleic acid molecule of claim 30, wherein the RNA molecule comprises at least one modified uridine.

34-35. (canceled)

36. The recombinant nucleic acid molecule of claim 33, wherein the modified uridine is N1-methylpseudouridine.

37. The recombinant nucleic acid molecule of claim 1, wherein at least one of the first polynucleotide and second polynucleotide is operably linked to a 3′ untranslated region (3′ UTR), or a polynucleotide encoding a 3′ UTR.

38-41. (canceled)

42. The recombinant nucleic acid molecule of claim 1, wherein the recombinant nucleic acid molecule further comprises one or more microRNA (miRNA) binding sites, or one or more polynucleotides encoding one or more miRNA binding sites.

43-45. (canceled)

46. The recombinant nucleic acid molecule of claim 42, wherein the one or more miRNA binding sites comprise a polynucleotide selected from SEQ ID NO: 36 and SEQ ID NO: 37.

47-48. (canceled)

49. The recombinant nucleic acid molecule of claim 1, wherein at least one of the first polynucleotide and second polynucleotide is operably linked to a 5′ untranslated region (5′ UTR), or a polynucleotide encoding a 5′ UTR.

50-51. (canceled)

52. The recombinant nucleic acid molecule of claim 9, wherein the 5′ UTR comprises SEQ ID NO: 33.

53-64. (canceled)

65. A lipid nanoparticle (LNP) comprising the recombinant nucleic acid molecule of claim 1.

66-68. (canceled)

69. A cell comprising the recombinant nucleic acid molecule of claim 1.

70-77. (canceled)

78. A pharmaceutical composition comprising the recombinant nucleic acid molecule of claim 1, and b) a pharmaceutically acceptable carrier.

79. A pharmaceutical composition comprising:
(a) two or more recombinant polynucleotides each encoding a different polypeptide, wherein at least one of the recombinant polynucleotides encodes TRIF or a variant thereof, and at least one of the recombinant polynucleotides encodes a polypeptide selected from the group consisting of: RIPK3, Gasdermin E, vICA, Npro, A238L, vMLKL, and variants thereof, and a dominant negative variant of IKBa; and
(b) a pharmaceutically acceptable carrier.

80. The pharmaceutical composition of claim 79, further comprising a recombinant polynucleotides encoding IL-12 or a variant thereof.

81. (canceled)

82. The pharmaceutical composition of claim 80, wherein each of the two or more recombinant polynucleotides in the pharmaceutical composition is comprised in a separate nucleic acid molecule.

83-84. (canceled)

85. The pharmaceutical composition of claim 79, wherein the two or more recombinant polynucleotides are RNA molecules.

86. The pharmaceutical composition of claim 85, wherein the RNA molecules are mRNAs.

87-88. (canceled)

89. A method of increasing immune response in a subject in need thereof, the method comprising administering the pharmaceutical composition of claim 78 to the subject in an amount and for a time sufficient to increase immune response in the subject.

90. The method of claim 89, wherein administration of the pharmaceutical composition to the subject increases immune response relative to a subject that is administered a pharmaceutical composition that comprises a polynucleotide encoding TRIF or a variant thereof, but does not comprise a polynucleotides encoding the additional polypeptide.

91. (canceled)

92. The method of claim 89, wherein the increasing immune response comprises:
a) increasing the expression and/or activity of one or more proteins selected from the group consisting of NFκB, IRF, NFAT, myd88, AP-1, STAT1, STAT2, STAT3, STAT 4, STAT 5, IRAK1, IRAK2, IRAK 3 and IRAK 4;
b) increasing cytokine or chemokine production and/or activity;
c) increasing immune cell mediated cytotoxicity;
d) increasing expression of a receptor-ligand pairing;
e) reducing anti-inflammatory signals and/or anti-inflammatory cells;
f) increased HLA/MHC antigen presentation or antigen release by target cells;
g) reduced expression of anti-immune factors; or
h) one or more of: activation of NK cells, activation of antigen-presenting dendritic cells, activation of CD4+ T cells, activation of CD8+ T cells, and conversion of immunosuppressive macrophages to immune-stimulatory macrophages.

93-107. (canceled)

108. A method of treating a cancer in a subject in need thereof, the method comprising administering the pharmaceutical composition of claim 78 to the subject in an amount and for a time sufficient to treat the cancer.

109-110. (canceled)

111. The method of claim 108, wherein administering the pharmaceutical composition to the subject reduces tumor growth and/or proliferation of cancer cells in the subject relative to a subject that is not administered the pharmaceutical composition.

112. (canceled)

113. The method of claim 108, wherein treating a cancer comprises any one or more of reduction in tumor burden, reduction in tumor size, inhibition of tumor growth, achievement of stable cancer in a subject with a progressive cancer prior to treatment, increased time to progression of the cancer, and increased time of survival.

114. The method of claim 108, wherein the cancer is a solid tumor.

115. The method of claim 108, wherein the cancer is selected from the group consisting of melanoma, colorectal cancer, lung cancer, head and neck cancer, gastric cancer, ovarian cancer, prostate cancer, adrenocortical cancer and breast cancer.

116. The method of claim 108, wherein the cancer is colon cancer or melanoma.

117. (canceled)

118. The method of claim 108, wherein the method further comprises administering an anti-neoplastic agent to the subject.

119. The method of claim 108, wherein administration of the pharmaceutical composition to the subject increases survival time and/or reduces tumor growth relative to a subject that is administered a pharmaceutical composition that comprises a polynucleotide encoding TRIF or a variant thereof, but does not comprise a polynucleotide encoding the additional polypeptide.

120. (canceled)


SEQUENCE LISTING (68 sequences)

SEQ ID NO: 1 - DNA, 2139 bp, Homo sapiens
  misc_feature 1..2139: note = Human wildtype TRIF nucleic acid sequence
  source 1..2139: mol_type = genomic DNA
atggcttgcacaggaccttctctgcccagcgcctttgatatcctgggagccgctggacaggacaagctgctgtacctgaagcacaagctgaaaacccctcggcctggctgccagggacaagatctgctgcatgctatggtgctgctgaagctgggccaagagacagaggccagaatcagcctggaagccctgaaggctgatgccgtggctagactggttgccagacaatgggctggcgtggacagcacagaggaccctgaagaacctcctgacgtgtcctgggccgtcgccagactgtatcatctgctggccgaagagaagctgtgccccgcctctctgagagatgtggcctatcaagaagccgtgcggaccctgagcagcagggatgatcatagactgggcgagctgcaggacgaggcccggaatagatgtggctgggatattgctggcgaccccggcagcattagaaccctgcagtctaacctgggctgcctgcctccatcttctgccctgccatctggcacaagaagcctgcctagacctatcgacggcgtgtccgattggagccagggctgttctctgagaagcacaggatctccagccagcctggccagcaacctggaaatcagccagtctcctacaatgcccttcctgagcctgcacagaagccctcacggacctagcaagctgtgcgacgatcctcaggcttctctggtgcctgaacctgttcctggcggctgccaagagcctgaagagatgtcttggcctcctagcggcgagatcgcctctccacctgaactgccatctagccctccaccaggactgcctgaagtggcccctgatgccacttctacaggcctgcctgatacacccgccgctccagagacaagcacaaactaccctgtggaatgcaccgagggctctgccggacctcaatctctgcctctgcctatcctggaacctgtgaagaacccttgcagcgtgaaggatcagacccctctgcagctgagcgtggaagataccacctctcctaacaccaagccttgtcctccaacacctaccacacctgagacaagcccacctcctccgcctccaccaccaagctctacaccttgtagcgcccacctgacaccaagcagcctgtttccaagctctctggaaagcagcagcgagcagaaattctacaacttcgtgatcctgcacgccagagccgacgagcacattgccctgagagtgcgcgaaaagctggaagctctgggagtgcctgatggcgccaccttctgcgaggattttcaggttcccggaagaggcgagctgagctgtctgcaggatgccatcgatcacagcgccttcatcattctgctgctgaccagcaacttcgactgccggctgtctctgcaccaagtgaaccaggccatgatgagcaacctgaccagacagggcagccccgattgcgtgatcccattcctgccactggaaagctccccagcacagctgtctagcgatactgcctctctgctgtctggactcgtgcggctggatgagcacagccagatcttcgccagaaaggtggccaacaccttcaagccccatcggctgcaggccagaaaagccatgtggcggaaagagcaggacacacgggcactgagagagcagtctcagcacctggatggcgagagaatgcaggccgctgctctgaatgccgcctacagcgcttacctgcagagctacctgagctatcaggcccagatggaacagctgcaggtcgcctttggcagccacatgtcctttggaacaggcgccccttacggcgccagaatgccttttggtggacaggtgccactgggagcccctccaccttttccaacatggccaggatgtccccagcctcctccactgcatgcttggcaagctggaacacctccgccaccatctccacagccagctgcctttccacagtctctcccatttccacagagccccgcctttccaacagctagccctgctcctccacaaagccctggactgcagcccctgatcattcaccacgcacagatggtgcagctgggactgaacaatcacatgtggaaccagagaggctctcaggcccctgaggacaagacacaagaggccgaatga

SEQ ID NO: 2 - protein, 712 aa, Homo sapiens
  REGION 1..712: note = misc_feature - Human wildtype TRIF amino acid sequence
MACTGPSLPSAFDILGAAGQDKLLYLKHKLKTPRPGCQGQDLLHAMVLLKLGQETEARISLEALKADAVARLVARQWAGVDSTEDPEEPPDVSWAVARLYHLLAEEKLCPASLRDVAYQEAVRTLSSRDDHRLGELQDEARNRCGWDIAGDPGSIRTLQSNLGCLPPSSALPSGTRSLPRPIDGVSDWSQGCSLRSTGSPASLASNLEISQSPTMPFLSLHRSPHGPSKLCDDPQASLVPEPVPGGCQEPEEMSWPPSGEIASPPELPSSPPPGLPEVAPDATSTGLPDTPAAPETSTNYPVECTEGSAGPQSLPLPILEPVKNPCSVKDQTPLQLSVEDTTSPNTKPCPPTPTTPETSPPPPPPPPSSTPCSAHLTPSSLFPSSLESSSEQKFYNFVILHARADEHIALRVREKLEALGVPDGATFCEDFQVPGRGELSCLQDAIDHSAFIILLLTSNFDCRLSLHQVNQAMMSNLTRQGSPDCVIPFLPLESSPAQLSSDTASLLSGLVRLDEHSQIFARKVANTFKPHRLQARKAMWRKEQDTRALREQSQHLDGERMQAAALNAAYSAYLQSYLSYQAQMEQLQVAFGSHMSFGTGAPYGARMPFGGQVPLGAPPPFPTWPGCPQPPPLHAWQAGTPPPPSPQPAAFPQSLPFPQSPAFPTASPAPPQSPGLQPLIIHHAQMVQLGLNNHMWNQRGSQAPEDKTQEAE

SEQ ID NO: 3 - DNA, 2139 bp, synthetic construct
  misc_feature 1..2139: note = Synthetic: TRIF mutant
  source 1..2139: mol_type = other DNA
atggcttgcacaggaccttctctgcccagcgcctttgatatcctgggagccgctggacaggacaagctgctgtacctgaagcacaagctgaaaacccctcggcctggctgccagggacaagatctgctgcatgctatggtgctgctgaagctgggccaagagacagaggccagaatcagcctggaagccctgaaggctgatgccgtggctagactggttgccagacaatgggctggcgtggacagcacagaggaccctgaagaacctcctgacgtgtcctgggccgtcgccagactgtatcatctgctggccgaagagaagctgtgccccgcctctctgagagatgtggcctatcaagaagccgtgcggaccctgagcagcagggatgatcatagactgggcgagctgcaggacgaggcccggaatagatgtggctgggatattgctggcgaccccggcagcattagaaccctgcagtctaacctgggctgcctgcctccatcttctgccctgccatctggcacaagaagcctgcctagacctatcgacggcgtgtccgattggagccagggctgttctctgagaagcacaggatctccagccagcctggccagcaacctggaaatcagccagtctcctacaatgcccttcctgagcctgcacagaagccctcacggacctagcaagctgtgcgacgatcctcaggcttctctggtgcctgaacctgttcctggcggctgccaagagcctgaagagatgtcttggcctcctagcggcgagatcgcctctccacctgaactgccatctagccctccaccaggactgcctgaagtggcccctgatgccacttctacaggcctgcctgatacacccgccgctccagagacaagcacaaactaccctgtggaatgcaccgagggctctgccggacctcaatctctgcctctgcctatcctggaacctgtgaagaacccttgcagcgtgaaggatcagacccctctgcagctgagcgtggaagataccacctctcctaacaccaagccttgtcctccaacacctaccacacctgagacaagcccacctcctccgcctccaccaccaagctctacaccttgtagcgcccacctgacaccaagcagcctgtttccaagctctctggaaagcagcagcgagcagaaattctacaacttcgtgatcctgcacgccagagccgacgagcacattgccctgagagtgcgcgaaaagctggaagctctgggagtgcctgatggcgccaccttctgcgaggattttcaggttcccggaagaggcgagctgagctgtctgcaggatgccatcgatcacagcgccttcatcattctgctgctgaccagcaacttcgactgccggctgtctctgcaccaagtgaaccaggccatgatgagcaacctgaccagacagggcagccccgattgcgtgatcccattcctgccactggaaagctccccagcacagctgtctagcgatactgcctctctgctgtctggactcgtgcggctggatgagcacagccagatcttcgccagaaaggtggccaacaccttcaagccccatcggctgcaggccagaaaagccatgtggcggaaagagcaggacacacgggcactgagagagcagtctcagcacctggatggcgagagaatgcaggccgctgctctgaatgccgcctacagcgcttacctgcagagctacctgagctatcaggcccagatggaacagctgcaggtcgcctttggcagccacatgtcctttggaacaggcgccccttacggcgccagaatgccttttggtggacaggtgccactgggagcccctccaccttttccaacatggccaggatgtccccagcctcctccactgcatgcttggcaagctggaacacctccgccaccatctccacagccagctgcctttccacagtctctcccatttccacagagccccgcctttccaacagctagccctgctcctccacaaagccctggactgcagcccctgatcattcaccacgcacagatggtggcagcggccgctaacaatcacatgtggaaccagagaggctctcaggcccctgaggacaagacacaagaggccgaatga

SEQ ID NO: 4 - protein, 712 aa, synthetic construct
  REGION 1..712: note = Synthetic: TRIF mutant
MACTGPSLPSAFDILGAAGQDKLLYLKHKLKTPRPGCQGQDLLHAMVLLKLGQETEARISLEALKADAVARLVARQWAGVDSTEDPEEPPDVSWAVARLYHLLAEEKLCPASLRDVAYQEAVRTLSSRDDHRLGELQDEARNRCGWDIAGDPGSIRTLQSNLGCLPPSSALPSGTRSLPRPIDGVSDWSQGCSLRSTGSPASLASNLEISQSPTMPFLSLHRSPHGPSKLCDDPQASLVPEPVPGGCQEPEEMSWPPSGEIASPPELPSSPPPGLPEVAPDATSTGLPDTPAAPETSTNYPVECTEGSAGPQSLPLPILEPVKNPCSVKDQTPLQLSVEDTTSPNTKPCPPTPTTPETSPPPPPPPPSSTPCSAHLTPSSLFPSSLESSSEQKFYNFVILHARADEHIALRVREKLEALGVPDGATFCEDFQVPGRGELSCLQDAIDHSAFIILLLTSNFDCRLSLHQVNQAMMSNLTRQGSPDCVIPFLPLESSPAQLSSDTASLLSGLVRLDEHSQIFARKVANTFKPHRLQARKAMWRKEQDTRALREQSQHLDGERMQAAALNAAYSAYLQSYLSYQAQMEQLQVAFGSHMSFGTGAPYGARMPFGGQVPLGAPPPFPTWPGCPQPPPLHAWQAGTPPPPSPQPAAFPQSLPFPQSPAFPTASPAPPQSPGLQPLIIHHAQMVAAAANNHMWNQRGSQAPEDKTQEAE

SEQ ID NO: 5 - DNA, 1623 bp, synthetic construct
  misc_feature 1..1623: note = Synthetic: TRIF mutant
  source 1..1623: mol_type = other DNA
atggcttgcacaggaccttctctgcccagcgcctttgatatcctgggagccgctggacaggacaagctgctgtacctgaagcacaagctgaaaacccctcggcctggctgccagggacaagatctgctgcatgctatggtgctgctgaagctgggccaagagacagaggccagaatcagcctggaagccctgaaggctgatgccgtggctagactggttgccagacaatgggctggcgtggacagcacagaggaccctgaagaacctcctgacgtgtcctgggccgtcgccagactgtatcatctgctggccgaagagaagctgtgccccgcctctctgagagatgtggcctatcaagaagccgtgcggaccctgagcagcagggatgatcatagactgggcgagctgcaggacgaggcccggaatagatgtggctgggatattgctggcgaccccggcagcattagaaccctgcagtctaacctgggctgcctgcctccatcttctgccctgccatctggcacaagaagcctgcctagacctatcgacggcgtgtccgattggagccagggctgttctctgagaagcacaggatctccagccagcctggccagcaacctggaaatcagccagtctcctacaatgcccttcctgagcctgcacagaagccctcacggacctagcaagctgtgcgacgatcctcaggcttctctggtgcctgaacctgttcctggcggctgccaagagcctgaagagatgtcttggcctcctagcggcgagatcgcctctccacctgaactgccatctagccctccaccaggactgcctgaagtggcccctgatgccacttctacaggcctgcctgatacacccgccgctccagagacaagcacaaactaccctgtggaatgcaccgagggctctgccggacctcaatctctgcctctgcctatcctggaacctgtgaagaacccttgcagcgtgaaggatcagacccctctgcagctgagcgtggaagataccacctctcctaacaccaagccttgtcctccaacacctaccacacctgagacaagcccacctcctccgcctccaccaccaagctctacaccttgtagcgcccacctgacaccaagcagcctgtttccaagctctctggaaagcagcagcgagcagaaattctacaacttcgtgatcctgcacgccagagccgacgagcacattgccctgagagtgcgcgaaaagctggaagctctgggagtgcctgatggcgccaccttctgcgaggattttcaggttcccggaagaggcgagctgagctgtctgcaggatgccatcgatcacagcgccttcatcattctgctgctgaccagcaacttcgactgccggctgtctctgcaccaagtgaaccaggccatgatgagcaacctgaccagacagggcagccccgattgcgtgatcccattcctgccactggaaagctccccagcacagctgtctagcgatactgcctctctgctgtctggactcgtgcggctggatgagcacagccagatcttcgccagaaaggtggccaacaccttcaagccccatcggctgcaggccagaaaagccatgtggtga

SEQ ID NO: 6 - protein, 540 aa, synthetic construct
  REGION 1..540: note = Synthetic: TRIF mutant
MACTGPSLPSAFDILGAAGQDKLLYLKHKLKTPRPGCQGQDLLHAMVLLKLGQETEARISLEALKADAVARLVARQWAGVDSTEDPEEPPDVSWAVARLYHLLAEEKLCPASLRDVAYQEAVRTLSSRDDHRLGELQDEARNRCGWDIAGDPGSIRTLQSNLGCLPPSSALPSGTRSLPRPIDGVSDWSQGCSLRSTGSPASLASNLEISQSPTMPFLSLHRSPHGPSKLCDDPQASLVPEPVPGGCQEPEEMSWPPSGEIASPPELPSSPPPGLPEVAPDATSTGLPDTPAAPETSTNYPVECTEGSAGPQSLPLPILEPVKNPCSVKDQTPLQLSVEDTTSPNTKPCPPTPTTPETSPPPPPPPPSSTPCSAHLTPSSLFPSSLESSSEQKFYNFVILHARADEHIALRVREKLEALGVPDGATFCEDFQVPGRGELSCLQDAIDHSAFIILLLTSNFDCRLSLHQVNQAMMSNLTRQGSPDCVIPFLPLESSPAQLSSDTASLLSGLVRLDEHSQIFARKVANTFKPHRLQARKAMW

SEQ ID NO: 7 - DNA, 2139 bp, synthetic construct
  misc_feature 1..2139: note = Synthetic: TRIF mutant
  source 1..2139: mol_type = other DNA
atggcttgcacaggaccttctctgcccagcgcctttgatatcctgggagccgctggacaggacaagctgctgtacctgaagcacaagctgaaaacccctcggcctggctgccagggacaagatctgctgcatgctatggtgctgctgaagctgggccaagagacagaggccagaatcagcctggaagccctgaaggctgatgccgtggctagactggttgccagacaatgggctggcgtggacagcacagaggaccctgaagaacctcctgacgtgtcctgggccgtcgccagactgtatcatctgctggccgaagagaagctgtgccccgcctctctgagagatgtggcctatcaagaagccgtgcggaccctgagcagcagggatgatcatagactgggcgagctgcaggacgaggcccggaatagatgtggctgggatattgctggcgaccccggcagcattagaaccctgcagtctaacctgggctgcctgcctccatcttctgccctgccatctggcacaagaagcctgcctagacctatcgacggcgtgtccgattggagccagggctgttctctgagaagcacaggatctccagccagcctggccagcaacctggaaatcgcacaggctcctgccatgcccttcctgagcctgcacagaagccctcacggacctagcaagctgtgcgacgatcctcaggcttctctggtgcctgaacctgttcctggcggctgccaagagcctgaagagatgtcttggcctcctagcggcgagatcgcctctccacctgaactgccatctagccctccaccaggactgcctgaagtggcccctgatgccacttctacaggcctgcctgatacacccgccgctccagagacaagcacaaactaccctgtggaatgcaccgagggctctgccggacctcaatctctgcctctgcctatcctggaacctgtgaagaacccttgcagcgtgaaggatcagacccctctgcagctgagcgtggaagataccacctctcctaacaccaagccttgtcctccaacacctaccacacctgagacaagcccacctcctccgcctccaccaccaagctctacaccttgtagcgcccacctgacaccaagcagcctgtttccaagctctctggaaagcagcagcgagcagaaattctacaacttcgtgatcctgcacgccagagccgacgagcacattgccctgagagtgcgcgaaaagctggaagctctgggagtgcctgatggcgccaccttctgcgaggattttcaggttcccggaagaggcgagctgagctgtctgcaggatgccatcgatcacagcgccttcatcattctgctgctgaccagcaacttcgactgccggctgtctctgcaccaagtgaaccaggccatgatgagcaacctgaccagacagggcagccccgattgcgtgatcccattcctgccactggaaagctccccagcacagctgtctagcgatactgcctctctgctgtctggactcgtgcggctggatgagcacagccagatcttcgccagaaaggtggccaacaccttcaagccccatcggctgcaggccagaaaagccatgtggcggaaagagcaggacacacgggcactgagagagcagtctcagcacctggatggcgagagaatgcaggccgctgctctgaatgccgcctacagcgcttacctgcagagctacctgagctatcaggcccagatggaacagctgcaggtcgcctttggcagccacatgtcctttggaacaggcgccccttacggcgccagaatgccttttggtggacaggtgccactgggagcccctccaccttttccaacatggccaggatgtccccagcctcctccactgcatgcttggcaagctggaacacctccgccaccatctccacagccagctgcctttccacagtctctcccatttccacagagccccgcctttccaacagctagccctgctcctccacaaagccctggactgcagcccctgatcattcaccacgcacagatggtgcagctgggactgaacaatcacatgtggaaccagagaggctctcaggcccctgaggacaagacacaagaggccgaatga

SEQ ID NO: 8 - protein, 712 aa, synthetic construct
  REGION 1..712: note = Synthetic: TRIF mutant
MACTGPSLPSAFDILGAAGQDKLLYLKHKLKTPRPGCQGQDLLHAMVLLKLGQETEARISLEALKADAVARLVARQWAGVDSTEDPEEPPDVSWAVARLYHLLAEEKLCPASLRDVAYQEAVRTLSSRDDHRLGELQDEARNRCGWDIAGDPGSIRTLQSNLGCLPPSSALPSGTRSLPRPIDGVSDWSQGCSLRSTGSPASLASNLEIAQAPAMPFLSLHRSPHGPSKLCDDPQASLVPEPVPGGCQEPEEMSWPPSGEIASPPELPSSPPPGLPEVAPDATSTGLPDTPAAPETSTNYPVECTEGSAGPQSLPLPILEPVKNPCSVKDQTPLQLSVEDTTSPNTKPCPPTPTTPETSPPPPPPPPSSTPCSAHLTPSSLFPSSLESSSEQKFYNFVILHARADEHIALRVREKLEALGVPDGATFCEDFQVPGRGELSCLQDAIDHSAFIILLLTSNFDCRLSLHQVNQAMMSNLTRQGSPDCVIPFLPLESSPAQLSSDTASLLSGLVRLDEHSQIFARKVANTFKPHRLQARKAMWRKEQDTRALREQSQHLDGERMQAAALNAAYSAYLQSYLSYQAQMEQLQVAFGSHMSFGTGAPYGARMPFGGQVPLGAPPPFPTWPGCPQPPPLHAWQAGTPPPPSPQPAAFPQSLPFPQSPAFPTASPAPPQSPGLQPLIIHHAQMVQLGLNNHMWNQRGSQAPEDKTQEAE

SEQ ID NO: 9 - DNA, 2139 bp, synthetic construct
  misc_feature 1..2139: note = Synthetic: TRIF mutant
  source 1..2139: mol_type = other DNA
atggcttgcacaggaccttctctgcccagcgcctttgatatcctgggagccgctggacaggacaagctgctgtacctgaagcacaagctgaaaacccctcggcctggctgccagggacaagatctgctgcatgctatggtgctgctgaagctgggccaagagacagaggccagaatcagcctggaagccctgaaggctgatgccgtggctagactggttgccagacaatgggctggcgtggacagcacagaggaccctgaagaacctcctgacgtgtcctgggccgtcgccagactgtatcatctgctggccgaagagaagctgtgccccgcctctctgagagatgtggcctatcaagaagccgtgcggaccctgagcagcagggatgatcatagactgggcgagctgcaggacgaggcccggaatagatgtggctgggatattgctggcgaccccggcagcattagaaccctgcagtctaacctgggctgcctgcctccatcttctgccctgccatctggcacaagaagcctgcctagacctatcgacggcgtgtccgattggagccagggctgttctctgagaagcacaggatctccagccagcctggccagcaacctggaaatcagccagtctcctacaatgcccttcctgagcctgcacagaagccctcacggacctagcaagctgtgcgacgatcctcaggcttctctggtgcctgaacctgttcctggcggctgccaagagcctgaagagatgtcttggcctcctagcggcgagatcgcctctccacctgaactgccatctagccctccaccaggactgcctgaagtggcccctgatgccacttctacaggcctgcctgatacacccgccgctccagagacaagcacaaactaccctgtggaatgcaccgagggctctgccggacctcaatctctgcctctgcctatcctggaacctgtgaagaacccttgcagcgtgaaggatcagacccctctgcagctgagcgtggaagataccacctctcctaacaccaagccttgtcctccaacacctaccacacctgagacaagcccacctcctccgcctccaccaccaagctctacaccttgtagcgcccacctgacaccaagcagcctgtttccaagctctctggaaagcagcagcgagcagaaattctacaacttcgtgatcctgcacgccagagccgacgagcacattgccctgagagtgcgcgaaaagctggaagctctgggagtgcctgatggcgccaccttctgcgaggattttcaggttcatggaagaggcgagctgagctgtctgcaggatgccatcgatcacagcgccttcatcattctgctgctgaccagcaacttcgactgccggctgtctctgcaccaagtgaaccaggccatgatgagcaacctgaccagacagggcagccccgattgcgtgatcccattcctgccactggaaagctccccagcacagctgtctagcgatactgcctctctgctgtctggactcgtgcggctggatgagcacagccagatcttcgccagaaaggtggccaacaccttcaagccccatcggctgcaggccagaaaagccatgtggcggaaagagcaggacacacgggcactgagagagcagtctcagcacctggatggcgagagaatgcaggccgctgctctgaatgccgcctacagcgcttacctgcagagctacctgagctatcaggcccagatggaacagctgcaggtcgcctttggcagccacatgtcctttggaacaggcgccccttacggcgccagaatgccttttggtggacaggtgccactgggagcccctccaccttttccaacatggccaggatgtccccagcctcctccactgcatgcttggcaagctggaacacctccgccaccatctccacagccagctgcctttccacagtctctcccatttccacagagccccgcctttccaacagctagccctgctcctccacaaagccctggactgcagcccctgatcattcaccacgcacagatggtgcagctgggactgaacaatcacatgtggaaccagagaggctctcaggcccctgaggacaagacacaagaggccgaatga

SEQ ID NO: 10 - protein, 712 aa, synthetic construct
  REGION 1..712: note = Synthetic: TRIF mutant
MACTGPSLPSAFDILGAAGQDKLLYLKHKLKTPRPGCQGQDLLHAMVLLKLGQETEARISLEALKADAVARLVARQWAGVDSTEDPEEPPDVSWAVARLYHLLAEEKLCPASLRDVAYQEAVRTLSSRDDHRLGELQDEARNRCGWDIAGDPGSIRTLQSNLGCLPPSSALPSGTRSLPRPIDGVSDWSQGCSLRSTGSPASLASNLEISQSPTMPFLSLHRSPHGPSKLCDDPQASLVPEPVPGGCQEPEEMSWPPSGEIASPPELPSSPPPGLPEVAPDATSTGLPDTPAAPETSTNYPVECTEGSAGPQSLPLPILEPVKNPCSVKDQTPLQLSVEDTTSPNTKPCPPTPTTPETSPPPPPPPPSSTPCSAHLTPSSLFPSSLESSSEQKFYNFVILHARADEHIALRVREKLEALGVPDGATFCEDFQVHGRGELSCLQDAIDHSAFIILLLTSNFDCRLSLHQVNQAMMSNLTRQGSPDCVIPFLPLESSPAQLSSDTASLLSGLVRLDEHSQIFARKVANTFKPHRLQARKAMWRKEQDTRALREQSQHLDGERMQAAALNAAYSAYLQSYLSYQAQMEQLQVAFGSHMSFGTGAPYGARMPFGGQVPLGAPPPFPTWPGCPQPPPLHAWQAGTPPPPSPQPAAFPQSLPFPQSPAFPTASPAPPQSPGLQPLIIHHAQMVQLGLNNHMWNQRGSQAPEDKTQEAE

SEQ ID NO: 11 - DNA, 1209 bp, synthetic construct
  misc_feature 1..1209: note = Synthetic: TRIF mutant
  source 1..1209: mol_type = other DNA
atgcagtctctgcccctgcctatcctggaacctgtgaagaacccctgcagcgtgaaggatcagacccctctgcagctgagcgtggaagataccacctctcctaacaccaagccttgtcctccaacacctaccacacctgagacaagccctccacctccgcctccaccaccaagctctacaccttgtagcgcccacctgacaccaagcagcctgtttccatccagcctggaaagcagcagcgagcagaaattctacaacttcgtgatcctgcacgccagagccgacgagcacattgccctgagagtgcgcgaaaagctggaagccctgggagttcctgatggcgccaccttctgcgaggatttccaagtgcctggaagaggcgagctgagctgtctgcaggatgccatcgatcacagcgccttcatcatcctgctgctgaccagcaacttcgactgcagactgagcctgcaccaagtgaaccaggccatgatgagcaacctgaccagacagggcagccccgattgcgtgatcccattcctgcctctggaaagctcccctgctcagctgtctagcgatacagcctctctgctgtctggactcgtgcggctggatgagcacagccagatcttcgccagaaaggtggccaacaccttcaagccccatcggctgcaggccagaaaagccatgtggcggaaagagcaggacacacgggctctgagagagcagtctcagcacctggatggcgagagaatgcaggccgctgctctgaatgccgcctactctgcctacctgcagagctacctgagctatcaggcccagatggaacagctgcaggtcgcctttggcagccacatgtcctttggaacaggcgccccttacggcgccagaatgccttttggtggacaggtgccactgggagcccctccaccatttcctacatggccaggatgtccccagcctcctccactgcatgcttggcaagctggaacaccacctcctccatctccacagcctgccgcctttccacagagcctgccttttccacagtctcccgcttttcccaccgcctctccagctccaccacaatctccaggactgcagcccctgatcatccaccacgctcaaatggtgcagctgggactgaacaatcacatgtggaaccagcggggctctcaggcccctgaggataagacacaagaggccgagtga

SEQ ID NO: 12 - protein, 402 aa, synthetic construct
  REGION 1..402: note = Synthetic: TRIF mutant
MQSLPLPILEPVKNPCSVKDQTPLQLSVEDTTSPNTKPCPPTPTTPETSPPPPPPPPSSTPCSAHLTPSSLFPSSLESSSEQKFYNFVILHARADEHIALRVREKLEALGVPDGATFCEDFQVPGRGELSCLQDAIDHSAFIILLLTSNFDCRLSLHQVNQAMMSNLTRQGSPDCVIPFLPLESSPAQLSSDTASLLSGLVRLDEHSQIFARKVANTFKPHRLQARKAMWRKEQDTRALREQSQHLDGERMQAAALNAAYSAYLQSYLSYQAQMEQLQVAFGSHMSFGTGAPYGARMPFGGQVPLGAPPPFPTWPGCPQPPPLHAWQAGTPPPPSPQPAAFPQSLPFPQSPAFPTASPAPPQSPGLQPLIIHHAQMVQLGLNNHMWNQRGSQAPEDKTQEAE

SEQ ID NO: 13 - DNA, 1602 bp, synthetic construct
  misc_feature 1..1602: note = Synthetic: TRIF mutant
  source 1..1602: mol_type = other DNA
atgcccatcgacggcgtgtccgattggagccagggctgttctctgcggagcaccggcagccctgccagcctggcctctaacctggaaatcagccaaagccccaccatgccattcctgtctcttcacagaagtccgcacggccctagcaagctgtgcgacgacccccaggctagcctggtgccagaacctgtgcctggcggctgccaggagcctgaggaaatgagctggcctccttctggcgagatcgcctccccgcccgagctgcctagctctcctcctcccggcctgcctgaagtggctcctgatgccacttctacaggactgcctgatacacccgccgcacccgaaaccagcaccaactaccccgtggaatgcaccgagggctcagccggcccccaatctctccctctgcctatcctggagcccgtgaaaaacccttgcagcgtgaaggaccagacccctctgcagctgagcgtggaggataccacaagccccaacacaaagccctgtccccctacccccacaacacctgagacaagcccaccacctcctcctccacctcctagcagtaccccttgtagcgcccatctgaccccatcaagcctgttccctagctctctagagagcagcagtgaacaaaagttctacaacttcgtgatcctgcacgccagagccgacgagcacatcgccctgagagtgcgggaaaagctggaggccctgggtgtgcctgacggcgccaccttctgcgaggacttccaggtccctggcagaggcgagctgtcctgtctgcaagacgccatcgaccactctgccttcatcatcctgctgctgacctccaatttcgactgccggctgagcctccaccaggttaatcaggccatgatgagcaacctgacaagacagggcagccccgactgcgtgatccctttcctgcctctggaaagcagccctgcccaactgtcgtctgatacagccagcctgctgagcggcctggtcagactggacgagcacagccagattttcgccagaaaagtggccaacaccttcaagcctcaccggctgcaggcccggaaagccatgtggcggaaggaacaggacaccagagccctgagagagcagagccagcacctggatggcgaaagaatgcaggccgctgccctgaatgccgcctacagcgcctacctgcagagctacctgtcttatcaggcccagatggaacagctgcaggtggcttttggcagccacatgtccttcggcaccggcgctccttacggtgccaggatgcccttcggaggccaggtgccactgggagcccctcctccttttcctacctggcccggctgcccccaacctccacctctgcatgcttggcaagctggaacacctccaccaccttctccaaagcctgccgcatttccccagtccctgccttttcctcagagccctgctttcccaaccgccagcccagctcctccccagagccctggactgcagcctctgatcatccaccacgcccagatggtgcagctcggactgaacaaccacatgtggaaccagagaggctctcaggctcctgaggacaagacacaggaggccgagtga

SEQ ID NO: 14 - protein, 533 aa, synthetic construct
  REGION 1..533: note = Synthetic: TRIF mutant
MPIDGVSDWSQGCSLRSTGSPASLASNLEISQSPTMPFLSLHRSPHGPSKLCDDPQASLVPEPVPGGCQEPEEMSWPPSGEIASPPELPSSPPPGLPEVAPDATSTGLPDTPAAPETSTNYPVECTEGSAGPQSLPLPILEPVKNPCSVKDQTPLQLSVEDTTSPNTKPCPPTPTTPETSPPPPPPPPSSTPCSAHLTPSSLFPSSLESSSEQKFYNFVILHARADEHIALRVREKLEALGVPDGATFCEDFQVPGRGELSCLQDAIDHSAFIILLLTSNFDCRLSLHQVNQAMMSNLTRQGSPDCVIPFLPLESSPAQLSSDTASLLSGLVRLDEHSQIFARKVANTFKPHRLQARKAMWRKEQDTRALREQSQHLDGERMQAAALNAAYSAYLQSYLSYQAQMEQLQVAFGSHMSFGTGAPYGARMPFGGQVPLGAPPPFPTWPGCPQPPPLHAWQAGTPPPPSPKPAAFPQSLPFPQSPAFPTASPAPPQSPGLQPLIIHHAQMVQLGLNNHMWNQRGSQAPEDKTQEAE

SEQ ID NO: 15 - DNA, 483 bp, synthetic construct
  misc_feature 1..483: note = Synthetic: TRIF mutant
  source 1..483: mol_type = other DNA
atggagtctagcagcgagcagaaattctacaacttcgtgatcctgcacgccagagccgacgagcacattgccctgagagtgcgcgaaaagctggaagccctgggagttcctgatggcgccaccttctgcgaggatttccaagtgcctggaagaggcgagctgagctgtctgcaggatgccatcgatcacagcgccttcatcatcctgctgctgaccagcaacttcgactgcagactgagcctgcaccaagtgaaccaggccatgatgagcaacctgaccagacagggcagccccgattgcgtgatcccattcctgcctctggaaagcagccctgctcagctgtctagcgatacagcctctctgctgtctggactcgtgcggctggatgagcacagccagatcttcgccagaaaggtggccaacaccttcaagccccatcggctgcaggccagaaaagccatgtggcggaaagagcaggactga

SEQ ID NO: 16 - protein, 159 aa, Homo sapiens
  REGION 1..159: note = misc_feature - Human wildtype TIR domain amino acid sequence
MESSSEQKFYNFVILHARADEHIALRVREKLEALGVPDGATFCEDFQVPGRGELSCLQDAIDHSAFIILLLTSNFDCRLSLHQVNQAMMSNLTRQGSPDCVIPFLPLESSPAQLSSDTASLLSGLVRLDEHSQIFARKVANTFKPHRLQARKAMWRKEQ

SEQ ID NO: 17 - DNA, 276 bp, synthetic construct
  misc_feature 1..276: note = Synthetic: TRIF mutant
  source 1..276: mol_type = other DNA
atgcctatcgacggcgtgtccgactggtcccaaggctgcagcctgagaagcaccggcagccctgctagcctggcctctaatctggaaatcagccaatctccaacaatgccccagagccctgccttccccaccgccagcccagctcctcctcagtctcccggcctgcagcctctgatcatccaccacgcccagatggtgcagctgggcctgaacaaccacatgtggaaccagcggggaagccaggcccctgaggacaaaacccaggaggccgaatga

SEQ ID NO: 18 - protein, 91 aa, synthetic construct
  REGION 1..91: note = Synthetic: TRIF mutant
MPIDGVSDWSQGCSLRSTGSPASLASNLEISQSPTMPQSPAFPTASPAPPQSPGLQPLIIHHAQMVQLGLNNHMWNQRGSQAPEDKTQEAE

SEQ ID NO: 19 - DNA, 591 bp, synthetic construct
  misc_feature 1..591: note = Synthetic: TRIF mutant
  source 1..591: mol_type = other DNA
atgcctatcgacggagtgtccgactggtcccagggatgtagccttagaagcaccggcagccctgctagcctggcctctaacctggaaatcagccagagccctacaatgccagagagcagcagcgagcagaaattctacaacttcgtgattctgcacgccagagccgacgagcacatcgccctgagagtgcgggaaaagctggaggccctgggcgtgcctgatggcgccaccttctgcgaagattttcaggtccccggcagaggcgagctgagctgcctgcaggacgctatcgaccacagcgccttcatcatcctgctgctgacaagcaacttcgactgcaggctgagcctgcaccaggtgaaccaggccatgatgagcaatctgaccagacagggctccccagattgcgtgatccccttcctgcctctggagagctctcctgcacagctgtcttctgacaccgccagcctgctctccggcctggtgcggctggacgagcattctcaaatcttcgccagaaaagtggccaacacctttaagccccacagactgcaagctcggaaggccatgtggcggaaggaacaggattga

SEQ ID NO: 20 - protein, 195 aa, synthetic construct
  REGION 1..195: note = Synthetic: TRIF mutant
MPIDGVSDWSQGCSLRSTGSPASLASNLEISQSPTMPESSSEQKFYNFVILHARADEHIALRVREKLEALGVPDGATFCEDFQVPGRGELSCLQDAIDHSAFIILLLTSNFDCRLSLHQVNQAMMSNLTRQGSPDCVIPFLPLESSPAQLSSDTASLLSGLVRLDEHSQIFARKVANTFKPHRLQARKAMWRKEQ

SEQ ID NO: 21 - DNA, 2166 bp, synthetic construct
  misc_feature 1..2166: note = Synthetic: TRIF mutant
  source 1..2166: mol_type = other DNA
atgcctatcgacggcgtgtccgattggagccagggatgttctctgcggagcacaggctctcccgcctctctggccagcaacctggaaatcagccaatctcctaccatgcccgaaagctcttctgagcagaagttctacaactttgtgatcctgcacgcccgggccgacgagcacatcgccctgagagtgcgggaaaagctggaagccctgggcgtgcccgacggcgcaacattttgtgaagatttccaggttcctggtagaggagagctttcctgtctgcaagacgccattgaccacagcgccttcatcatcctgctgctgacatctaacttcgactgcagactgtccctgcaccaggtgaatcaggccatgatgagcaatctgaccagacagggcagccctgactgcgtgatccccttcctgccactggaaagcagccctgctcagctgagtagcgacaccgccagcctgctcagcggcctggtcagactggacgaacactctcaaatcttcgccagaaaagtggctaataccttcaagccccaccggctgcaggccagaaaggctatgtggcggaaagaacaggatggccccggcggatccagcggcagcagctgcgtcaaactgtggcctagcggcgcccctgcccctctggtgtccatcgaggaactggaaaaccaagagcttgtcggcaagggcggcttcggcactgttttcagagctcaacacagaaagtggggctacgacgtggccgtgaagatcgtgaacagcaaggccatcagcagagaagtgaaggccatggccagcctggacaacgagttcgtgctgagactggagggagtgatcgagaaggtgaactgggaccaggatcctaaacccgccctggtgacaaagttcatggaaaacggcagcctgagcggactgctgcagagccaatgtcctagaccttggcccctgctgtgccggctcctgaaggaagtggtgctcggcatgttctacctgcatgatcagaatcccgtgctgctgcacagagatctgaaaccctccaacgtgctgctggatcctgagctgcacgtgaagctggccgatttcggcctgagcacctttcagggcggcagtcagagcggcacaggcagcggcgagcctggcggcaccctgggctacctggctcctgagctgttcgtgaatgtgaatcggaaggccagcacagcttctgacgtgtatagctttggcatcctgatgtgggccgtgctggctggaagggaagttgagctgcccaccgagcccagcctggtgtacgaggccgtgtgcaaccggcagaaccggccaagcctggccgagctgcctcaggctggccctgagacacctggccttgagggcctgaaggagctcatgcagctgtgctggagctccgagccaaaggacagaccatctttccaggagtgcctgcctaagaccgacgaggtgttccagatggtggaaaacaacatgaacgccgccgtcagcaccgtgaaggactttctgagccagctgagatcttccaatagacgcttcagcatccctgagtctggacagggaggaacagagatggacggattccggagaaccatcgagaaccagcactcccggaacgacgtgatggtcagcgagtggctgaacaagctgaacctggaagagcctccaagctcagtgcccaagaagtgcccctctctgaccaaaagaagcagagcccaggaggaacaggtgcctcaggcctggaccgccggaacaagcagcgacagcatggcccagcctccgcaaacacctgaaaccagcaccttcagaaaccagatgcctagccccaccagcaccggcaccccttcccctggccccagaggcaaccagggcgcagagcggcagggcatgaactggtcctgccgcacccccgagcccaaccctgtgaccggccggcctctggtgaacatctacaactgcagcggtgtgcaggtgggtgataacaattacctgaccatgcagcagaccaccgctctgccaacatggggcctcgccccttctggcaagggcagaggcctgcagcatcctcctcctgtgggctctcaggaaggacctaaggaccccgaggcctggagcaggcctcagggctggtacaaccactcaggcaaatga

SEQ ID NO: 22 - protein, 721 aa, synthetic construct
  REGION 1..721: note = Synthetic: TRIF mutant
MPIDGVSDWSQGCSLRSTGSPASLASNLEISQSPTMPESSSEQKFYNFVILHARADEHIALRVREKLEALGVPDGATFCEDFQVPGRGELSCLQDAIDHSAFIILLLTSNFDCRLSLHQVNQAMMSNLTRQGSPDCVIPFLPLESSPAQLSSDTASLLSGLVRLDEHSQIFARKVANTFKPHRLQARKAMWRKEQDGPGGSSGSSCVKLWPSGAPAPLVSIEELENQELVGKGGFGTVFRAQHRKWGYDVAVKIVNSKAISREVKAMASLDNEFVLRLEGVIEKVNWDQDPKPALVTKFMENGSLSGLLQSQCPRPWPLLCRLLKEVVLGMFYLHDQNPVLLHRDLKPSNVLLDPELHVKLADFGLSTFQGGSQSGTGSGEPGGTLGYLAPELFVNVNRKASTASDVYSFGILMWAVLAGREVELPTEPSLVYEAVCNRQNRPSLAELPQAGPETPGLEGLKELMQLCWSSEPKDRPSFQECLPKTDEVFQMVENNMNAAVSTVKDFLSQLRSSNRRFSIPESGQGGTEMDGFRRTIENQHSRNDVMVSEWLNKLNLEEPPSSVPKKCPSLTKRSRAQEEQVPQAWTAGTSSDSMAQPPQTPETSTFRNQMPSPTSTGTPSPGPRGNQGAERQGMNWSCRTPEPNPVTGRPLVNIYNCSGVQVGDNNYLTMQQTTALPTWGLAPSGKGRGLQHPPPVGSQEGPKDPEAWSRPQGWYNHSGK

SEQ ID NO: 23 - DNA, 24 bp, synthetic construct
  misc_feature 1..24: note = Synthetic: FLAG-tag
  source 1..24: mol_type = other DNA
gactacaaggacgacgacgacaag

SEQ ID NO: 24 - protein, 8 aa, synthetic construct
  REGION 1..8: note = Synthetic: FLAG-tag
DYKDDDDK

SEQ ID NO: 25 - protein, 9 aa, synthetic construct
  REGION 1..9: note = Synthetic: linker
GPGGSSGSS

SEQ ID NO: 26 - protein, 18 aa, synthetic construct
  REGION 1..18: note = Synthetic: T2A peptide
EGRGSLLTCGDVEENPGP

SEQ ID NO: 27 - protein, 19 aa, synthetic construct
  REGION 1..19: note = Synthetic: P2A peptide
ATNFSLLKQAGDVEENPGP

SEQ ID NO: 28 - protein, 20 aa, synthetic construct
  REGION 1..20: note = Synthetic: E2A peptide
QCTNYALLKLAGDVESNPGP

SEQ ID NO: 29 - protein, 22 aa, synthetic construct
  REGION 1..22: note = Synthetic: F2A peptide
VKQTLNFDLLKLAGDVESNPGP

SEQ ID NO: 30 - protein, 518 aa, Homo sapiens
  REGION 1..518: note = misc_feature - Human wildtype RIPK3 amino acid sequence
MSCVKLWPSGAPAPLVSIEELENQELVGKGGFGTVFRAQHRKWGYDVAVKIVNSKAISREVKAMASLDNEFVLRLEGVIEKVNWDQDPKPALVTKFMENGSLSGLLQSQCPRPWPLLCRLLKEVVLGMFYLHDQNPVLLHRDLKPSNVLLDPELHVKLADFGLSTFQGGSQSGTGSGEPGGTLGYLAPELFVNVNRKASTASDVYSFGILMWAVLAGREVELPTEPSLVYEAVCNRQNRPSLAELPQAGPETPGLEGLKELMQLCWSSEPKDRPSFQECLPKTDEVFQMVENNMNAAVSTVKDFLSQLRSSNRRFSIPESGQGGTEMDGFRRTIENQHSRNDVMVSEWLNKLNLEEPPSSVPKKCPSLTKRSRAQEEQVPQAWTAGTSSDSMAQPPQTPETSTFRNQMPSPTSTGTPSPGPRGNQGAERQGMNWSCRTPEPNPVTGRPLVNIYNCSGVQVGDNNYLTMQQTTALPTWGLAPSGKGRGLQHPPPVGSQEGPKDPEAWSRPQGWYNHSGK

SEQ ID NO: 31 - DNA, 1557 bp, Homo sapiens
  misc_feature 1..1557: note = Human wildtype RIPK3 nucleic acid sequence
  source 1..1557: mol_type = genomic DNA
atgtcgtgcgtcaagttatggcccagcggtgcccccgcccccttggtgtccatcgaggaactggagaaccaggagctcgtcggcaaaggcgggttcggcacagtgttccgggcgcaacataggaagtggggctacgatgtggcggtcaagatcgtaaactcgaaggcgatatccagggaggtcaaggccatggcaagtctggataacgaattcgtgctgcgcctagaaggggttatcgagaaggtgaactgggaccaagatcccaagccggctctggtgactaaattcatggagaacggctccttgtcggggctgctgcagtcccagtgccctcggccctggccgctcctttgccgcctgctgaaagaagtggtgcttgggatgttttacctgcacgaccagaacccggtgctcctgcaccgggacctcaagccatccaacgtcctgctggacccagagctgcacgtcaagctggcagattttggcctgtccacatttcagggaggctcacagtcagggacagggtccggggagccagggggcaccctgggctacttggccccagaactgtttgttaacgtaaaccggaaggcctccacagccagtgacgtctacagcttcgggatcctaatgtgggcagtgcttgctggaagagaagttgagttgccaaccgaaccatcactcgtgtacgaagcagtgtgcaacaggcagaaccggccttcattggctgagctgccccaagccgggcctgagactcccggcttagaaggactgaaggagctaatgcagctctgctggagcagtgagcccaaggacagaccctccttccaggaatgcctaccaaaaactgatgaagtcttccagatggtggagaacaatatgaatgctgctgtctccacggtaaaggatttcctgtctcagctcaggagcagcaataggagattttctatcccagagtcaggccaaggagggacagaaatggatggctttaggagaaccatagaaaaccagcactctcgtaatgatgtcatggtttctgagtggctaaacaaactgaatctagaggagcctcccagctctgttcctaaaaaatgcccgagccttaccaagaggagcagggcacaagaggagcaggttccacaagcctggacagcaggcacatcttcagattcgatggcccaacctccccagactccagagacctcaactttcagaaaccagatgcccagccctacctcaactggaacaccaagtcctggaccccgagggaatcagggggctgagagacaaggcatgaactggtcctgcaggaccccggagccaaatccagtaacagggcgaccgctcgttaacatatacaactgctctggggtgcaagttggagacaacaactacttgactatgcaacagacaactgccttgcccacatggggcttggcaccttcgggcaaggggaggggcttgcagcaccccccaccagtaggttcgcaagaaggccctaaagatcctgaagcctggagcaggccacagggttggtataatcatagcgggaaataa

SEQ ID NO: 32 - protein, 476 aa, synthetic construct
  REGION 1..476: note = Synthetic: vICA
MDDLRDTLMAYGCIAIRAGDFNGLNDFLEQECGTRLHVAWPERCFIQLRSRSALGPFVGKMGTVCSQGAYVCCQEYLHPFGFVEGPGFMRYQLIVLIGQRGGIYCYDDLRDCIYELAPTMKDFLRHGFRHCDHFHTMRDYQRPMVQYDDYWNAVMLYRGDVESLSAEVTKRGYASYSIDDPFDECPDTHFAFWTHNTEVMKFKETSFSVVRAGGSIQTMELMIRTVPRITCYHQLLGALGHEVPERKEFLVRQYVLVDTFGVVYGYDPAMDAVYRLAEDVVMFTCVMGKKGHRNHRFSGRREAIVRLEKTPTCQHPKKTPDPMIMFDEDDDDELSLPRNVMTHEEAESRLYDAITENLMHCVKLVTTDSPLATHLWPQELQALCDSPALSLCTDDVEGVRQKLRARTGSLHHFELSYRFHDEDPETYMGFLWDIPSCDRCVRRRRFKVCDVGRRHIIPGAANGMPPLTPPHAYMNN

SEQ ID NO: 33 - RNA, 57 nt, synthetic construct
  misc_feature 1..57: note = Synthetic: 5 UTR RNA sequence
  source 1..57: mol_type = other RNA
gggaaataagagagaaaagaagagtaagaagaaatataagaccccggcgccgccacc

SEQ ID NO: 34 - RNA, 152 nt, synthetic construct
  misc_feature 1..152: note = Synthetic: 3 UTR RNA sequence
  source 1..152: mol_type = other RNA
taatgataataggctggagcctcggtggccatgcttcttgccccttgggcctccccccagcccctcctccccttcctgcacccgtaccccccaaacaccattgtcacactccagtggtctttgaataaagtctgagtgggcggcgcggccgc

SEQ ID NO: 35 - RNA, 172 nt, synthetic construct
  misc_feature 1..172: note = Synthetic: 3 UTR RNA sequence
  source 1..172: mol_type = other RNA
tgataatagtccataaagtaggaaacactacagctggagcctcggtggccatgcttcttgccccttgggcccaaacaccattgtcacactccatccccccagcccctcctccccttcctgcacccgtacccccgtggtctttgaataaagtctgagtgggcggcgcggccgc

SEQ ID NO: 36 - RNA, 22 nt, synthetic construct
  misc_feature 1..22: note = Synthetic: miR-122-5p binding site RNA sequence
  source 1..22: mol_type = other RNA
caaacaccattgtcacactcca

SEQ ID NO: 37 - RNA, 23 nt, synthetic construct
  misc_feature 1..23: note = Synthetic: miR-142-3p binding site RNA sequence
  source 1..23: mol_type = other RNA
tccataaagtaggaaacactaca

SEQ ID NO: 38 - DNA, 130 bp, synthetic construct
  misc_feature 1..130: note = Synthetic: PolyA tail
  source 1..130: mol_type = other DNA
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 39 - DNA, 2475 bp, synthetic construct
  misc_feature 1..2475: note = Synthetic: TRIF DNA construct
  source 1..2475: mol_type = other DNA
gggaaataagagagaaaagaagagtaagaagaaatataagaccccggcgccgccaccatggcctgtaccggcccctccctcccctcggccttcgacatcctgggagcagcgggccaagacaagctcctgtacctgaagcacaagctgaaaactcctaggcccggatgtcagggccaggatctgctgcacgcgatggtgctgctcaagctgggtcaagaaaccgaggcccgcatcagcctggaagccttgaaagcggacgccgtggccaggctcgtggcccggcagtgggccggagtggactccactgaggatccggaagaacccccggatgtgtcgtgggcggtggctcgcctgtaccacctcctggctgaagagaagttatgcccagcctccctccgcgacgtggcataccaggaggccgtgcggaccctctcctcgcgggacgaccatcgcctcggtgaactgcaggatgaagctcgcaacagatgtggctgggatattgctggcgatccgggatccattcggacactgcagagcaaccttggatgcctgcccccatcgtccgcgctgccgagcggcacccgatccctgccacgcccgattgacggagtctccgactggtcacagggatgcagcctccggtccaccggatccccggcctcactcgcaagcaacctggaaatctcgcagtccccaactatgccgttcctgtcactgcatcgctcccctcatggaccatccaagctgtgcgacgatccacaggcctccctcgtgcctgaacctgtgccgggaggctgccaggaaccggaggagatgtcctggccgcctagcggggagatcgcctcaccgcctgaactcccgtcctccccccctccgggtttgcccgaagtggcccctgacgcgaccagcaccggactgcccgacactcccgccgcgcccgaaacctccactaattacccggtggaatgcaccgaggggtcggcagggccacagtccctgcctctgcctattctggagcctgtgaagaatccctgctctgtgaaggaccagactcctctgcaactgtcagtggaggataccacctcgcccaacacgaagccctgccctcctactccaactacccctgagacttcgcctccgccgcctcccccgccaccctcctccaccccgtgttccgcccacctcacaccttcctccctgtttccgtcatccctggaaagttcgtcggaacagaaattctacaacttcgtgatcctccacgcgcgggctgatgagcatatcgccttgcgcgtgcgggagaagttggaagcactgggggtgcccgacggtgccaccttctgcgaggattttcaggtccccggaaggggagagctcagctgcctgcaagacgccatcgaccacagcgccttcatcatcctccttctgacctcaaacttcgactgccgcctgagcctgcaccaggtcaaccaggccatgatgtccaacctgaccagacagggctcacccgactgtgtgattccgttcctgccactggagagcagccccgcccagctgtcctccgacaccgcctcactactgtccggccttgtccggctggatgaacacagccagatcttcgcaagaaaggtcgcgaacaccttcaagccgcacagactgcaggcccggaaggccatgtggagaaaggagcaggacacgagagcccttcgggagcagtcccagcatcttgacggggaacgcatgcaggccgctgccttgaacgcggcgtacagcgcctatctgcaatcgtacctgtcctaccaagcacagatggagcagctccaagtggctttcggaagccacatgagctttggaactggtgcaccgtacggcgccaggatgcccttcggcggacaagtccctctgggtgcccccccaccgttcccaacctggcccgggtgccctcagccgcccccgcttcacgcttggcaggccggcactcctcctcccccgtcccctcaaccggccgctttcccgcaatcccttcccttcccacaatctcccgccttcccgactgcctccccggccccgcctcaatccccgggactccagcctttgatcattcaccacgcgcaaatggtccagctggggctgaacaaccatatgtggaaccagcggggctcgcaggcccccgaggacaagacccaggaagcagagtaatgataataggctggagcctcggtggccatgcttcttgccccttgggcctccccccagcccctcctccccttcctgcacccgtaccccccaaacaccattgtcacactccagtggtctttgaataaagtctgagtgggcggcgcggccgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 40 - DNA, 1827 bp, synthetic construct
  misc_feature 1..1827: note = Synthetic: GSDME DNA construct
  source 1..1827: mol_type = other DNA
gggaaataagagagaaaagaagagtaagaagaaatataagaccccggcgccgccaccatgttcgccaaggccaccagaaacttcctgagggaagtggacgccgacggggacttgattgcggtgtccaacctcaacgactccgacaagctccagctgcttagcctggtcaccaagaagaagcggttctggtgctggcagcggcccaagtaccagttcctgtcgctgacactaggcgacgtcttgatagaggaccagttcccctccccggtggtcgtggaatccgatttcgtgaaatatgaggggaagttcgccaatcacgtgtccggaactctggaaactgccctgggaaaggtcaagttgaacctgggcggaagctctagggtggaatcgcagtcctcctttggtaccctgcgcaagcaggaagtggatctgcagcagctcattcgggatagcgccgagcgcaccatcaacctgagaaaccccgtgctccaacaagtgctggaagggagaaacgaagtgctctgtgtgctgactcagaagatcactacgatgcagaaatgcgtgatctcagagcatatgcaagtcgaggagaaatgcggaggaatcgtaggaattcagaccaagactgtccaagtgtccgccactgaggacggaaacgtgaccaaggactcaaacgtggtgctggaaatccccgccgcgaccaccatcgcttacggcgtgatcgagctttacgtgaagctggacggacagttcgagttctgtctgctgcgggggaagcagggcggtttcgaaaacaagaagcggatcgatagcgtgtacctggacccgctggtgttccgcgagttcgcgtttattgacatgcctgacgccgcacacggcatctcgtcacaagacgggccactcagcgtgctgaagcaggccaccctgctgctcgagcggaatttccatccattcgctgagctcccggaaccccagcagactgccctgtccgacattttccaagccgtgctgttcgacgacgaactgttaatggtgctcgagcctgtgtgcgatgatctggtgtccggcctgtccccaaccgtggccgtcctgggcgaactcaagcctaggcagcagcaggacctggtggcgttccttcaactcgtcggatgctcgctgcaaggaggctgccctggacctgaggatgccggttccaagcagctgttcatgacggcctacttcctggtgtcggccctggccgaaatgccggactccgcagcagcgcttttgggcacctgttgcaaactgcagatcattccgaccctgtgccaccttctgcgcgccctgagcgatgatggagtgtcagacttggaggaccccaccctcactccgctgaaggacaccgaacggttcggcatcgtgcagagactgtttgcttccgcggatatctccctggaacgcctgaagtccagcgtcaaggccgtgattctgaaggactcgaaggtctttccgctgcttttgtgcatcaccctcaacggactctgcgctctcggtcgcgagcacagctaatgataataggctggagcctcggtggccatgcttcttgccccttgggcctccccccagcccctcctccccttcctgcacccgtaccccccaaacaccattgtcacactccagtggtctttgaataaagtctgagtgggcggcgcggccgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 41 - DNA, 1797 bp, synthetic construct
  misc_feature 1..1797: note = Synthetic: RIPK3 DNA construct
  source 1..1797: mol_type = other DNA
gggaaataagagagaaaagaagagtaagaagaaatataagaccccggcgccgccaccatgtcgtccgtcaagctgtggccaaccggagctagtgccgtgcctcttgtgtcccgggaggagctcaagaagttggagttcgtgggaaagggaggattcggcgtcgtgttccgggcgcaccacagaacttggaaccatgacgtggccgtgaagatcgtgaactcgaagaagatctcctgggaagtcaaggccatggtcaacttgcgcaacgagaatgtgctgctcctgctgggcgtcaccgaggatctgcagtgggactttgtgtccggtcaagccctcgtcacccgcttcatggagaacggatccctggccgggctgcttcagccggaatgtccgcgcccgtggccgctgctgtgccggctgctgcaagaagtggtgctggggatgtgctatcttcactcgctgaacccgcccttgttacaccgcgacctgaagccaagcaacattctgctggatcccgagctccacgccaagctggccgacttcggactgtcaacgttccaaggcggtagccagtcgggttccggatcgggatcaggatcgagggattcaggagggaccctggcctacctggacccggaactcctgttcgatgtgaacctgaaggcgtccaaggcctccgacgtgtacagctttgggatcctcgtctgggcagtgctggccggtcgagaagcggagcttgtggacaagaccagcctgattagggaaactgtgtgcgatcgccagtcccggcctcctctgactgaactcccgcccggatccccggaaaccccaggactggagaagctgaaggagctgatgatccactgctgggggtcccagagcgaaaacagaccatcattccaagactgcgaacctaagactaacgaagtgtacaatctcgtgaaagataaggtcgacgctgccgtgtccgaggtcaagcattacctaagccagcacaggtcgtccggccgcaacctcagcgcgcgggagcccagccagagaggcaccgagatggactgcccgcgggaaaccatggtgtccaaaatgctggacaggttgcacctggaagaaccctcaggaccggtgccaggaaagtgtccagagagacaggcccaggacacaagcgtgggccctgcaacccccgctcggacttcctccgatccggtggccggcaccccgcaaatccctcacaccctgccgttccgcggcaccacccccggccctgtgttcaccgaaactccggggccccatccccaacggaaccagggcgacgggagacacggtactccttggtacccctggacccctcctaaccctatgaccggtccgcccgccctcgtgttcaacaactgctccgaggtgcagattggcaactacaattcgctcgtggccccgccccggactaccgcttcctcctctgcgaaatacgaccaggcacagtttggccgcggacggggatggcagcccttccataagtaatgataataggctggagcctcggtggccatgcttcttgccccttgggcctccccccagcccctcctccccttcctgcacccgtaccccccaaacaccattgtcacactccagtggtctttgaataaagtctgagtgggcggcgcggccgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 42 - DNA, 1893 bp, synthetic construct
  misc_feature 1..1893: note = Synthetic: RIPK3 DNA construct
  source 1..1893: mol_type = other DNA
gggaaataagagagaaaagaagagtaagaagaaatataagaccccggcgccgccaccatgagctgtgtgaagctgtggccttccggcgccccagcccctctggtgtccattgaggaactggaaaaccaggagctggtcggaaagggcggattcggcacagtgttcagagcccagcaccggaagtggggatacgacgtggccgtgaaaatcgtgaacagcaaggccatcagcagagaggtgaaagccatggcctctctggataatgagttcgtgctgcggctggaaggagtgatcgaaaaggtgaattgggaccaggaccctaagcccgcactggtgaccaagttcatggaaaacggcagcctgagcggcctacttcagtctcaatgtcctagaccctggcctctgctgtgtagactgctgaaagaggtggtgctgggcatgttctacctgcatgatcagaaccccgtgctgctgcacagagatctgaagccttctaatgtgctgctggaccccgagctgcacgtgaagctcgctgatttcggcctgtctacattccagggcggctcgcagagcggcactggcagcggcgaacctggcgggaccctgggatatctggcccctgaactcttcgtgaacgtgaacagaaaggccagcacagctagcgacgtgtacagcttcggcatcctgatgtgggccgtgctggccggcagagaagtggaactgcccaccgagcctagcctggtgtacgaggccgtgtgcaaccgccagaaccggcctagcctggctgaactgcctcaagctggcccggaaacccctggcctcgaaggcctgaaggaactgatgcagctgtgctggtcctctgagccgaaggacagaccttcctttcaggagtgcctgcctaagaccgacgaggtctttcagatggtggaaaacaacatgaacgccgccgtcagcaccgtgaaggactttctgagccaactgagaagcagcaacagaaggttcagcatccccgagtccggacagggcggcacagagatggacggcttcagacggaccatcgagaatcagcacagcagaaacgacgtgatggtcagcgagtggctgaacaagctgaacctggaggaaccccctagcagcgtgcccaaaaagtgcccttccctgacaaagcggagccgggcccaggaggaacaggttcctcaggcctggaccgccggcaccagctctgacagcatggcccagcctcctcaaacaccagagacaagtaccttccggaaccagatgccttctcccaccagcaccggcacccctagccccggccctagaggcaaccagggcgccgaacggcagggcatgaactggagctgcagaacacctgagcccaaccctgtgacaggcagacccctggtgaatatctacaattgcagcggcgtgcaggtgggagataacaactacctgaccatgcagcagaccaccgccctgccaacctggggcctggctccatctggcaaaggccgggggctgcagcacccccctcctgtgggcagccaggagggacctaaggatcctgaggcctggagcagaccacagggatggtacaatcacagcggaaaataatgataataggctggagcctcggtggccatgcttcttgccccttgggcctccccccagcccctcctccccttcctgcacccgtaccccccaaacaccattgtcacactccagtggtctttgaataaagtctgagtgggcggcgcggccgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 43 - DNA, 1767 bp, synthetic construct
  misc_feature 1..1767: note = Synthetic: vICA DNA construct
  source 1..1767: mol_type = other DNA
gggaaataagagagaaaagaagagtaagaagaaatataagaccccggcgccgccaccatggatgatctgcgcgacaccttgatggcctacggctgcatcgccattcgcgccggagatttcaacggcctgaatgacttcctggagcaagagtgcggaaccagacttcacgtggcctggccggaacggtgcttcatccaactgcggtcgcgctcagcactgggacctttcgtggggaagatgggaaccgtgtgtagccagggtgcatacgtgtgctgtcaggagtatctgcacccattcggctttgtggaaggccccggcttcatgcggtaccagctgattgtgctgatcggccagcgcggcggaatctactgctacgatgacctcagagactgcatctacgaactggcccctactatgaaggacttcctgagacacggcttccgccattgcgatcatttccacaccatgcgcgactatcagcggccgatggtgcagtacgacgactactggaacgctgtgatgctctaccggggagatgtggagagcctgtccgccgaagtgaccaagaggggttacgctagctactccatcgacgatcccttcgacgaatgtcccgacactcacttcgccttctggacccataacaccgaggtcatgaagtttaaggaaacctcgttctccgtggtccgggccggagggtctatccagaccatggagctgatgattagaaccgtgcctcggatcacgtgttaccaccaacttctcggcgccttgggtcacgaagtgcctgaacggaaggagttcctcgtgcggcaatacgtgctggtcgacaccttcggcgtggtgtacggttacgaccctgcgatggacgcggtgtaccgcctcgccgaggacgtcgtcatgttcacttgcgtgatggggaagaagggacatcggaaccaccgcttctcgggaaggagggaagctatcgtccgcctggaaaagaccccgacgtgccagcacccgaagaaaacacccgatccaatgatcatgttcgacgaggatgacgatgacgagctgagcctgcccagaaacgtgatgacccacgaagaagccgaatcccggctctacgacgcaattaccgagaacctcatgcattgcgtgaagcttgtcactactgattcccctctggccactcacttgtggccgcaagagctgcaggccctctgcgactccccggcgctgtccctgtgcaccgacgacgtggaaggagtgcgccagaagctgagagccaggactggatccctgcaccactttgagctgtcctatcgattccacgacgaggacccggaaacctacatggggttcctgtgggacattccatcatgcgatagatgcgtgcgccggcggaggtttaaagtctgtgacgtgggacgcaggcacatcattcccggagcggcgaatgggatgccgcctctcactcccccgcatgcctacatgaacaactaatgataataggctggagcctcggtggccatgcttcttgccccttgggcctccccccagcccctcctccccttcctgcacccgtaccccccaaacaccattgtcacactccagtggtctttgaataaagtctgagtgggcggcgcggccgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 44 - DNA, 1488 bp, Homo sapiens
  misc_feature 1..1488: note = Human wildtype Gasdermin E nucleic acid sequence
  source 1..1488: mol_type = genomic DNA
atgttcgccaaggccaccagaaacttcctgagggaagtggacgccgacggggacttgattgcggtgtccaacctcaacgactccgacaagctccagctgcttagcctggtcaccaagaagaagcggttctggtgctggcagcggcccaagtaccagttcctgtcgctgacactaggcgacgtcttgatagaggaccagttcccctccccggtggtcgtggaatccgatttcgtgaaatatgaggggaagttcgccaatcacgtgtccggaactctggaaactgccctgggaaaggtcaagttgaacctgggcggaagctctagggtggaatcgcagtcctcctttggtaccctgcgcaagcaggaagtggatctgcagcagctcattcgggatagcgccgagcgcaccatcaacctgagaaaccccgtgctccaacaagtgctggaagggagaaacgaagtgctctgtgtgctgactcagaagatcactacgatgcagaaatgcgtgatctcagagcatatgcaagtcgaggagaaatgcggaggaatcgtaggaattcagaccaagactgtccaagtgtccgccactgaggacggaaacgtgaccaaggactcaaacgtggtgctggaaatccccgccgcgaccaccatcgcttacggcgtgatcgagctttacgtgaagctggacggacagttcgagttctgtctgctgcgggggaagcagggcggtttcgaaaacaagaagcggatcgatagcgtgtacctggacccgctggtgttccgcgagttcgcgtttattgacatgcctgacgccgcacacggcatctcgtcacaagacgggccactcagcgtgctgaagcaggccaccctgctgctcgagcggaatttccatccattcgctgagctcccggaaccccagcagactgccctgtccgacattttccaagccgtgctgttcgacgacgaactgttaatggtgctcgagcctgtgtgcgatgatctggtgtccggcctgtccccaaccgtggccgtcctgggcgaactcaagcctaggcagcagcaggacctggtggcgttccttcaactcgtcggatgctcgctgcaaggaggctgccctggacctgaggatgccggttccaagcagctgttcatgacggcctacttcctggtgtcggccctggccgaaatgccggactccgcagcagcgcttttgggcacctgttgcaaactgcagatcattccgaccctgtgccaccttctgcgcgccctgagcgatgatggagtgtcagacttggaggaccccaccctcactccgctgaaggacaccgaacggttcggcatcgtgcagagactgtttgcttccgcggatatctccctggaacgcctgaagtccagcgtcaaggccgtgattctgaaggactcgaaggtctttccgctgcttttgtgcatcaccctcaacggactctgcgctctcggtcgcgagcacagc

SEQ ID NO: 45 - DNA, 504 bp, synthetic construct
  misc_feature 1..504: note = Synthetic: Npro
  source 1..504: mol_type = other DNA
atggaactgaaccatttcgagctgctgtacaagacaagcaagcagaagcctgtgggcgtggaagagcctgtgtacgacaccgccggccggcctctgttcggcaaccccagcgaagtgcacccccagtctacacttaaactgccacacgatagaggccggggcgatatcagaacaacactgagagatctgcctagaaagggcgactgtagaagcggcaatcacctgggccctgtgtccggaatctacattaagcccggccccgtgtactaccaggactacaccggcccagtttatcacagagcccctctcgagttcttcgacgaggctcaattttgcgaggtgaccaagagaatcggcagagtgaccggaagcgacggcaagctgtaccacatctacgtgtgcgtggacggatgtatcctgctgaagctggccaagcggggaacccctcgcaccctgaaatggatccggaacttcaccaactgccctctgtgggtcaccagctgc

SEQ ID NO: 46 - DNA, 717 bp, synthetic construct
  misc_feature 1..717: note = Synthetic: A238L
  source 1..717: mol_type = other DNA
atggacaccatcggcctgttcagcgttgaagccgagcacctgtttgtggaatgggtcaagaagtgcatcaagaagggagatctgaccctgttcgagacactgtttaacgccgatccttggatcgtgaatagatgcaacaagaacaaaatcaccgtgtttatgctgatctgtatctacggaagactggacttcctccgcttcctgtttaagcaggagagctaccccggcgagatcgtgaaccactatagacgggacaaggacggcaactccgcctggcattacctggccgaaaagaacaaccacctgctgctggaagaagtgctggattacttcggcaagaatggcatcagggtgtgcttccctaatttcaacggcgtgacccctatcatgaaggccgctatgcggggcagaacactttctgtgctgagcctgctgaaatacggagctaatcctaaccggaaggactacctgaagggcttcacaacttgggattgggccgtgttcaccggccacgccgacctggtcaagaccctgaacaagggctaccagaagccactgttcatgcacttccccctgtacaagctggacgtgttccacagaagattcaagaaaaagcccaagatcatcattaccggctgcgaggacaacgtgtacgagaagctgcctgagcagaacagcaacttcctgtgtgtgaaaaaactgaacaagtacggcaaa

SEQ ID NO: 47 - DNA, 885 bp, synthetic construct
  misc_feature 1..885: note = Synthetic: vMLKL
  source 1..885: mol_type = other DNA
atgaccgaccccctgctgcacaagctctttccagtcgccgttaactccaacatcaaagtgatcaaggacacagatgtggtgaaggtggaaaatgagaacatctgcgagtgcaaaaactcttatgtgtacaacgccatctacaacaacgaggaagccctggtgatcatgttcaaacacagccatacaggctacaaggtgctgctggacatcagcgagaagtacaatattctgcaacagaagcacgagaagaacgtgctgagaatctacgccgtgttcatcgacaacagctacatcaacttcaagctgcctaagatctctctgatcgtggaaaagtgcaagcctatcagacggctgatcctggaaaagatcaacctgcctttcgagagcaagattgacctgatcctgaacgctctggatagcctgattctcctgtacacctacacaaaggagccatacaagtacatcaactccagcaacttcctgatcaattctgattacgacgtgaaaatcgtgtctcacggactggaaatcatcctgagcaatcctgcctttaacaccatcaagagcagctcctactacagccccaagatcatcatgaaccccttcagcaagcagaccatcagcgacgacatctatagcttcggcatcctgatgtacgagatcctgagctacaaaatccccttcgaaaacatgaaatcaagagatattatcagctgcgtggagagcaacaagatcgagagcatccttaatatcagctctatcaactgtcctgacaccctgaagaacctgatcaagaagtgtgtgtccatcgatcctgtcatccggccttcttttaaggaaatctaccagaacatcagcatctacaagttcaataagaaaattgag

SEQ ID NO: 48 - DNA, 1428 bp, synthetic construct
  misc_feature 1..1428: note = Synthetic: vICA
  source 1..1428: mol_type = other DNA
atggacgatctgagagatacactgatggcctatggatgtatcgccatcagagccggcgatttcaacggcctgaacgacttcctggagcaggagtgtggaacaagactgcatgtggcctggcctgagcggtgcttcatccagctgcggtctagaagcgccctcggccctttcgtgggaaaaatggggaccgtgtgcagccagggcgcctacgtgtgctgccaagaatacctgcacccttttggcttcgtggaaggacctggcttcatgcggtaccagcttatcgtgctgatcggccagagaggcggcatctactgctacgacgacctgagagactgcatctacgagctggcccctacaatgaaggactttctgagacacggctttagacactgtgatcactttcacacaatgagagattaccagcggcctatggtgcagtacgacgactactggaatgccgtgatgctgtaccggggcgacgtggaaagcctgtccgccgaggtgacaaagagaggctacgcctcttatagcatcgacgaccccttcgacgagtgccccgacacccacttcgccttctggacccacaacaccgaggtcatgaaattcaaggaaacctccttcagcgtggtccgggctggcggaagcattcagaccatggaactgatgatcaggaccgtgcccagaatcacatgctaccaccagctgctgggcgctctgggccacgaggttccagaaagaaaggagttcctggtcagacagtacgtgctggtggacaccttcggcgtggtgtacggatatgaccccgctatggatgctgtgtaccgcctggctgaggacgtggtgatgttcacctgcgtgatgggcaagaaaggccacagaaatcaccggttcagcggcagacgggaagccatcgtgcggctggaaaagacccctacctgtcaacaccctaagaagacacctgatccaatgatcatgttcgacgaggatgatgacgatgaactgagcctgcctagaaacgtgatgacccacgaggaagccgagagcagactgtacgacgccattaccgagaacctgatgcactgcgttaagctggtgaccaccgatagccccctggccacacatctgtggccccaggagctgcaggccctgtgcgacagccctgccctgtccctgtgtacagatgacgtggagggcgttagacagaagctgcgggccagaaccggcagcttgcaccatttcgagctgtcttacagattccacgacgaggaccctgaaacctacatgggattcctgtgggacatcccatcttgtgatagatgcgtgagaagaagacgctttaaggtgtgcgacgtgggcagaaggcacatcatccccggcgccgccaacggcatgcctccactgacccctcctcacgcctacatgaacaac

SEQ ID NO: 49 - DNA, 951 bp, synthetic construct
  misc_feature 1..951: note = Synthetic: IKBa dominant negative variant
  source 1..951: mol_type = other DNA
atgttccaggccgctgaaagacctcaggagtgggccatggaaggccctagggatggcctgaagaaggagcggctgctggatgatagacacgacgccggcctggacgccatgaaagacgaagagtacgagcagatggtgaaggaactgcaggagatcagactggaaccacaagaggtgcccagaggaagcgaaccctggaagcagcagctgaccgaggacggcgactctttcctgcacctggccatcatccacgaggagaaggccctgaccatggaagtgatccggcaggttaagggcgatctggcttttctgaacttccaaaacaacctgcaacaaacacctctgcacctggctgtgatcaccaatcagcctgagatcgccgaggccctgctgggagccggctgtgatcctgagctgcgggacttccgcggcaacacccctctgcatctggcttgtgaacagggctgcctggcctctgtgggcgtgctgacacagtcttgcaccacaccccacctgcacagcatcctgaaagccacaaattacaacggccacacctgcctgcacttggccagcatccacggatacctgggtattgtggaactgctggtgtccctgggcgctgatgtgaacgcccaggagccttgcaacggcagaacagccctgcacctggccgtggacctgcagaaccccgacctggtcagcctgctactcaagtgcggcgccgacgtgaatagagtgacctaccagggctacagcccttatcagctgacctggggccggcccagcaccagaatccagcagcagcttggacagctgactctggaaaacctgcagatgctcccagagtccgaagatgaggaaagctacgacaccgagagcgagttcaccgagtttaccgaggacgagctgccttacgacgactgcgtgttcggcggacagagactgacactg

SEQ ID NO: 50 - protein, 317 aa, Homo sapiens
  REGION 1..317: note = misc_feature - IKBa human wildtype amino acid sequence
MFQAAERPQEWAMEGPRDGLKKERLLDDRHDSGLDSMKDEEYEQMVKELQEIRLEPQEVPRGSEPWKQQLTEDGDSFLHLAIIHEEKALTMEVIRQVKGDLAFLNFQNNLQQTPLHLAVITNQPEIAEALLGAGCDPELRDFRGNTPLHLACEQGCLASVGVLTQSCTTPHLHSILKATNYNGHTCLHLASIHGYLGIVELLVSLGADVNAQEPCNGRTALHLAVDLQNPDLVSLLLKCGADVNRVTYQGYSPYQLTWGRPSTRIQQQLGQLTLENLQMLPESEDEESYDTESEFTEFTEDELPYDDCVFGGQRLTL

SEQ ID NO: 51 - DNA, 1551 bp, Homo sapiens
  misc_feature 1..1551: note = Human wildtype Caspase 1 full-length nucleic acid sequence
  source 1..1551: mol_type = genomic DNA
atgggcgtccaggtggaaaccatcagccctggcgacggcagaacatttccaaagcggggccagacttgtgtggtgcactacaccggcatgctggaagatggaaaaaaggtggactccagcagagatagaaacaaaccttttaagttcatgctgggcaagcaggaggtgatccggggatgggaggaaggcgtggcccagatgagcgtgggccagcgggccaagctcacaatcagcccagactacgcctacggcgccaccggacaccctggcatcatccccccccacgccacactggtgtttgacgttgagctgctgaagctggaaggctctggaagccccgctgctgacaaagtgctgaaagagaagcgcaagctgtttataaggtccatgggcgagggcacaatcaacggcctgctggacgagctgctgcagaccagagtgctgaacaaggaagagatggaaaaagtgaagagagagaacgccaccgtgatggacaagaccagggcccttatcgacagcgtgatccctaagggcgcacaagcctgtcagatctgtatcacatacatctgcgaggaggattcttatctggccggcaccctgggactgtctgccgatcagacaagcggcaattacctgaacatgcaggacagccagggcgtgctgtcttctttccctgcccctcaggccgtgcaggataatcctgccatgcctaccagctctggatctgagggaaacgtgaagttgtgcagcctggaagaggcccaaagaatctggaagcaaaagtccgccgagatctaccccatcatggacaagagcagcagaaccagactggccctgatcatctgcaacgaggagttcgacagcatccccagaagaaccggcgctgaagtggatatcaccggcatgaccatgctgctgcagaacctgggctacagcgtggacgtgaaaaagaacctgaccgccagcgacatgacaaccgagctggaagccttcgcccatagacctgagcacaagacctctgactctacattcctggtgttcatgagccacggtatccgggaaggcatctgcggcaagaagcacagcgagcaggttcctgacatcctgcaactgaatgccatcttcaacatgctcaacaccaagaactgccccagcctgaaggataagcctaaggtgatcatcattcaggcttgtagaggcgatagcccaggcgtcgtgtggttcaaggacagcgtcggcgtgtccggcaatctgagcctgcctacaaccgaagaattcgaggacgacgccatcaagaaagcccacatcgagaaagacttcatcgctttttgcagcagcacccctgataacgtgtcctggcggcaccccacaatgggcagcgtgttcattggcagactgatcgagcacatgcaggagtacgcctgctcatgcgacgtggaagaaatcttccggaaggtgcggttcagcttcgagcagcctgacggaagagcgcagatgcctaccaccgagagagtgaccctgacacggtgcttctacctgttccccggccac

SEQ ID NO: 52 - protein, 517 aa, Homo sapiens
  REGION 1..517: note = misc_feature - Human wildtype Caspase 1 full-length amino acid sequence
MGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLEGSGSPAADKVLKEKRKLFIRSMGEGTINGLLDELLQTRVLNKEEMEKVKRENATVMDKTRALIDSVIPKGAQACQICITYICEEDSYLAGTLGLSADQTSGNYLNMQDSQGVLSSFPAPQAVQDNPAMPTSSGSEGNVKLCSLEEAQRIWKQKSAEIYPIMDKSSRTRLALIICNEEFDSIPRRTGAEVDITGMTMLLQNLGYSVDVKKNLTASDMTTELEAFAHRPEHKTSDSTFLVFMSHGIREGICGKKHSEQVPDILQLNAIFNMLNTKNCPSLKDKPKVIIIQACRGDSPGVVWFKDSVGVSGNLSLPTTEEFEDDAIKKAHIEKDFIAFCSSTPDNVSWRHPTMGSVFIGRLIEHMQEYACSCDVEEIFRKVRFSFEQPDGRAQMPTTERVTLTRCFYLFPGH

SEQ ID NO: 53 - protein, 103 aa, Homo sapiens
  REGION 1..103: note = misc_feature - Human Caspase 1 fragment amino acid sequence
ADKVLKEKRKLFIRSMGEGTINGLLDELLQTRVLNKEEMEKVKRENATVMDKTRALIDSVIPKGAQACQICITYICEEDSYLAGTLGLSADQTSGNYLNMQDS

SEQ ID NO: 54 - protein, 103 aa, Homo sapiens
  REGION 1..103: note = misc_feature - Human Caspase 1 fragment
ADKVLKEKRKLFIRSMGEGTINGLLDELLQTRVLNKEEMEKVKRENATVMDKTRALIDSVIPKGAQACQICITYICEEDSYLAGTLGLSADLSNNGQVKPHTS

SEQ ID NO: 55 - DNA, 309 bp, synthetic construct
  misc_feature 1..309: note = Synthetic: CASP1FV
  source 1..309: mol_type = other DNA
gctgacaaagtgctgaaagagaagcgcaagctgtttataaggtccatgggcgagggcacaatcaacggcctgctggacgagctgctgcagaccagagtgctgaacaaggaagagatggaaaaagtgaagagagagaacgccaccgtgatggacaagaccagggcccttatcgacagcgtgatccctaagggcgcacaagcctgtcagatctgtatcacatacatctgcgaggaggattcttatctggccggcaccctgggactgtctgccgatttatctaataatggtcaagttaaacctcatacttct

SEQ ID NO: 56 - DNA, 1005 bp, Mus musculus
  misc_feature 1..1005: note = Mouse IL-12 p40 subunit nucleic acid sequence
  source 1..1005: mol_type = genomic DNA
atgtgccctcaaaagctcaccatttcctggttcgccatcgtgcttctggtgtccccgctgatggctatgtgggagcttgaaaaggatgtgtatgtggtggaagtggactggacccccgacgcgcctggagaaaccgtgaacctgacttgcgacacccctgaggaggacgacatcacatggactagcgaccagagacacggagtgattgggagtggaaagaccctcaccatcaccgtgaaggagttcctggatgccggacagtacacttgtcacaaggggggagagactctgagccattcgcatctgctgctccacaagaaggagaacggcatctggtccactgaaatcctgaagaacttcaagaacaagaccttccttaagtgcgaggcgcccaactactcgggaaggttcacgtgctcatggctggtccagagaaacatggaccttaagttcaacatcaagagctcctcgagctcccccgactctcgcgctgtgacttgtggcatggcttccttgtccgccgagaaggtcaccctggatcagcgggactacgaaaagtacagcgtgtcgtgccaagaagatgtgacctgtcctaccgccgaggaaaccctgccgatcgaactggccctggaagcgcggcagcagaataagtacgaaaactactccacctcgttctttatccgggacattatcaagcccgatcccccgaagaacctccagatgaagccgctgaagaacagccaagtggaggtgtcatgggagtatccagactcctggtccaccccgcactcctacttctccctgaaattcttcgtgcggatccagcggaagaaggaaaagatgaaggagactgaagaaggctgcaatcagaagggggcctttctggtggagaaaacttccaccgaggtccagtgcaaaggcggaaacgtctgtgtgcaagcacaggaccgatactacaactcctcttgctccaaatgggcgtgcgtgccttgccgcgtgcggtcc

SEQ ID NO: 57 - DNA, 579 bp, Mus musculus
  misc_feature 1..579: note = Mouse IL-12 p35 subunit nucleic acid sequence
  source 1..579: mol_type = genomic DNA
cgcgtgatccctgtgtccggccccgcccgctgcctgagccagtcgaggaacctactgaaaaccaccgacgacatggtcaagaccgccagagaaaagctgaagcattactcctgcaccgcggaggacatcgaccacgaggacattacgcgcgatcagacctcaacgctcaaaacttgcttgccgctcgagctgcacaagaacgaaagctgcctggccactagagaaactagctccactacccggggttcgtgtctgccgccccaaaagacctcactcatgatgaccctctgcctgggctcgatctacgaggatctgaagatgtaccagacagagttccaggccatcaacgccgcactgcaaaaccacaatcaccagcagatcattttggacaagggcatgctcgtggccattgacgaattgatgcagtccctcaaccataacggcgaaactctgaggcagaagccaccggtcggcgaagcagatccataccgcgtcaagatgaagctctgcatcctgctgcacgccttttcaacccgggtggtcaccattaaccgcgtgatgggttacctgagctccgcc

SEQ ID NO: 58 - DNA, 45 bp, synthetic construct
  misc_feature 1..45: note = Synthetic: linker
  source 1..45: mol_type = other DNA
ggaggaggagggagcggtggcggagggtcgggtggaggaggttcc

SEQ ID NO: 59 - protein, 15 aa, synthetic construct
  REGION 1..15: note = Synthetic: linker
GGGGSGGGGSGGGGS

SEQ ID NO: 60 - protein, 253 aa, Homo sapiens
  REGION 1..253: note = misc_feature - Human IL12A (p35) amino acid sequence
MWPPGSASQPPPSPAAATGLHPAARPVSLQCRLSMCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 61 - DNA, 762 bp, Homo sapiens
  misc_feature 1..762: note = Human IL12A (p35) nucleic acid sequence
  source 1..762: mol_type = genomic DNA
atgtggccccctgggtcagcctcccagccaccgccctcacctgccgcggccacaggtctgcatccagcggctcgccctgtgtccctgcagtgccggctcagcatgtgtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaa

SEQ ID NO: 62 - protein, 328 aa, Homo sapiens
  REGION 1..328: note = misc_feature - Human IL12B (p40) amino acid sequence
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 63 - DNA, 1007 bp, Homo sapiens
  misc_feature 1..1007: note = Human IL12B (p40) nucleic acid sequence
  source 1..1007: mol_type = genomic DNA
agcaagatgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagttaggttctgatccagga

SEQ ID NO: 64 - DNA, 1968 bp, synthetic construct
  misc_feature 1..1968: note = Synthetic: mIL-12 mRNA construct
  source 1..1968: mol_type = other DNA
gggaaataagagagaaaagaagagtaagaagaaatataagaccccggcgccgccaccatgtgccctcaaaagctcaccatttcctggttcgccatcgtgcttctggtgtccccgctgatggctatgtgggagcttgaaaaggatgtgtatgtggtggaagtggactggacccccgacgcgcctggagaaaccgtgaacctgacttgcgacacccctgaggaggacgacatcacatggactagcgaccagagacacggagtgattgggagtggaaagaccctcaccatcaccgtgaaggagttcctggatgccggacagtacacttgtcacaaggggggagagactctgagccattcgcatctgctgctccacaagaaggagaacggcatctggtccactgaaatcctgaagaacttcaagaacaagaccttccttaagtgcgaggcgcccaactactcgggaaggttcacgtgctcatggctggtccagagaaacatggaccttaagttcaacatcaagagctcctcgagctcccccgactctcgcgctgtgacttgtggcatggcttccttgtccgccgagaaggtcaccctggatcagcgggactacgaaaagtacagcgtgtcgtgccaagaagatgtgacctgtcctaccgccgaggaaaccctgccgatcgaactggccctggaagcgcggcagcagaataagtacgaaaactactccacctcgttctttatccgggacattatcaagcccgatcccccgaagaacctccagatgaagccgctgaagaacagccaagtggaggtgtcatgggagtatccagactcctggtccaccccgcactcctacttctccctgaaattcttcgtgcggatccagcggaagaaggaaaagatgaaggagactgaagaaggctgcaatcagaagggggcctttctggtggagaaaacttccaccgaggtccagtgcaaaggcggaaacgtctgtgtgcaagcacaggaccgatactacaactcctcttgctccaaatgggcgtgcgtgccttgccgcgtgcggtccggaggaggagggagcggtggcggagggtcgggtggaggaggttcccgcgtgatccctgtgtccggccccgcccgctgcctgagccagtcgaggaacctactgaaaaccaccgacgacatggtcaagaccgccagagaaaagctgaagcattactcctgcaccgcggaggacatcgaccacgaggacattacgcgcgatcagacctcaacgctcaaaacttgcttgccgctcgagctgcacaagaacgaaagctgcctggccactagagaaactagctccactacccggggttcgtgtctgccgccccaaaagacctcactcatgatgaccctctgcctgggctcgatctacgaggatctgaagatgtaccagacagagttccaggccatcaacgccgcactgcaaaaccacaatcaccagcagatcattttggacaagggcatgctcgtggccattgacgaattgatgcagtccctcaaccataacggcgaaactctgaggcagaagccaccggtcggcgaagcagatccataccgcgtcaagatgaagctctgcatcctgctgcacgccttttcaacccgggtggtcaccattaaccgcgtgatgggttacctgagctccgcctaatgataataggctggagcctcggtggccatgcttcttgccccttgggcctccccccagcccctcctccccttcctgcacccgtaccccccaaacaccattgtcacactccagtggtctttgaataaagtctgagtgggcggcgcggccgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 65 - DNA, 2495 bp, synthetic construct
  misc_feature 1..2495: note = Synthetic: hTRIF mRNA construct
  source 1..2495: mol_type = other DNA
gggaaataagagagaaaagaagagtaagaagaaatataagaccccggcgccgccaccatggcctgtaccggcccctccctcccctcggccttcgacatcctgggagcagcgggccaagacaagctcctgtacctgaagcacaagctgaaaactcctaggcccggatgtcagggccaggatctgctgcacgcgatggtgctgctcaagctgggtcaagaaaccgaggcccgcatcagcctggaagccttgaaagcggacgccgtggccaggctcgtggcccggcagtgggccggagtggactccactgaggatccggaagaacccccggatgtgtcgtgggcggtggctcgcctgtaccacctcctggctgaagagaagttatgcccagcctccctccgcgacgtggcataccaggaggccgtgcggaccctctcctcgcgggacgaccatcgcctcggtgaactgcaggatgaagctcgcaacagatgtggctgggatattgctggcgatccgggatccattcggacactgcagagcaaccttggatgcctgcccccatcgtccgcgctgccgagcggcacccgatccctgccacgcccgattgacggagtctccgactggtcacagggatgcagcctccggtccaccggatccccggcctcactcgcaagcaacctggaaatctcgcagtccccaactatgccgttcctgtcactgcatcgctcccctcatggaccatccaagctgtgcgacgatccacaggcctccctcgtgcctgaacctgtgccgggaggctgccaggaaccggaggagatgtcctggccgcctagcggggagatcgcctcaccgcctgaactcccgtcctccccccctccgggtttgcccgaagtggcccctgacgcgaccagcaccggactgcccgacactcccgccgcgcccgaaacctccactaattacccggtggaatgcaccgaggggtcggcagggccacagtccctgcctctgcctattctggagcctgtgaagaatccctgctctgtgaaggaccagactcctctgcaactgtcagtggaggataccacctcgcccaacacgaagccctgccctcctactccaactacccctgagacttcgcctccgccgcctcccccgccaccctcctccaccccgtgttccgcccacctcacaccttcctccctgtttccgtcatccctggaaagttcgtcggaacagaaattctacaacttcgtgatcctccacgcgcgggctgatgagcatatcgccttgcgcgtgcgggagaagttggaagcactgggggtgcccgacggtgccaccttctgcgaggattttcaggtccccggaaggggagagctcagctgcctgcaagacgccatcgaccacagcgccttcatcatcctccttctgacctcaaacttcgactgccgcctgagcctgcaccaggtcaaccaggccatgatgtccaacctgaccagacagggctcacccgactgtgtgattccgttcctgccactggagagcagccccgcccagctgtcctccgacaccgcctcactactgtccggccttgtccggctggatgaacacagccagatcttcgcaagaaaggtcgcgaacaccttcaagccgcacagactgcaggcccggaaggccatgtggagaaaggagcaggacacgagagcccttcgggagcagtcccagcatcttgacggggaacgcatgcaggccgctgccttgaacgcggcgtacagcgcctatctgcaatcgtacctgtcctaccaagcacagatggagcagctccaagtggctttcggaagccacatgagctttggaactggtgcaccgtacggcgccaggatgcccttcggcggacaagtccctctgggtgcccccccaccgttcccaacctggcccgggtgccctcagccgcccccgcttcacgcttggcaggccggcactcctcctcccccgtcccctcaaccggccgctttcccgcaatcccttcccttcccacaatctcccgccttcccgactgcctccccggccccgcctcaatccccgggactccagcctttgatcattcaccacgcgcaaatggtccagctggggctgaacaaccatatgtggaaccagcggggctcgcaggcccccgaggacaagacccaggaagcagagtgataatagtccataaagtaggaaacactacagctggagcctcggtggccatgcttcttgccccttgggcccaaacaccattgtcacactccatccccccagcccctcctccccttcctgcacccgtacccccgtggtctttgaataaagtctgagtgggcggcgcggccgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 66 - DNA, 1847 bp, synthetic construct
  misc_feature 1..1847: note = Synthetic: hGSDME mRNA construct
  source 1..1847: mol_type = other DNA
gggaaataagagagaaaagaagagtaagaagaaatataagaccccggcgccgccaccatgttcgccaaggccaccagaaacttcctgagggaagtggacgccgacggggacttgattgcggtgtccaacctcaacgactccgacaagctccagctgcttagcctggtcaccaagaagaagcggttctggtgctggcagcggcccaagtaccagttcctgtcgctgacactaggcgacgtcttgatagaggaccagttcccctccccggtggtcgtggaatccgatttcgtgaaatatgaggggaagttcgccaatcacgtgtccggaactctggaaactgccctgggaaaggtcaagttgaacctgggcggaagctctagggtggaatcgcagtcctcctttggtaccctgcgcaagcaggaagtggatctgcagcagctcattcgggatagcgccgagcgcaccatcaacctgagaaaccccgtgctccaacaagtgctggaagggagaaacgaagtgctctgtgtgctgactcagaagatcactacgatgcagaaatgcgtgatctcagagcatatgcaagtcgaggagaaatgcggaggaatcgtaggaattcagaccaagactgtccaagtgtccgccactgaggacggaaacgtgaccaaggactcaaacgtggtgctggaaatccccgccgcgaccaccatcgcttacggcgtgatcgagctttacgtgaagctggacggacagttcgagttctgtctgctgcgggggaagcagggcggtttcgaaaacaagaagcggatcgatagcgtgtacctggacccgctggtgttccgcgagttcgcgtttattgacatgcctgacgccgcacacggcatctcgtcacaagacgggccactcagcgtgctgaagcaggccaccctgctgctcgagcggaatttccatccattcgctgagctcccggaaccccagcagactgccctgtccgacattttccaagccgtgctgttcgacgacgaactgttaatggtgctcgagcctgtgtgcgatgatctggtgtccggcctgtccccaaccgtggccgtcctgggcgaactcaagcctaggcagcagcaggacctggtggcgttccttcaactcgtcggatgctcgctgcaaggaggctgccctggacctgaggatgccggttccaagcagctgttcatgacggcctacttcctggtgtcggccctggccgaaatgccggactccgcagcagcgcttttgggcacctgttgcaaactgcagatcattccgaccctgtgccaccttctgcgcgccctgagcgatgatggagtgtcagacttggaggaccccaccctcactccgctgaaggacaccgaacggttcggcatcgtgcagagactgtttgcttccgcggatatctccctggaacgcctgaagtccagcgtcaaggccgtgattctgaaggactcgaaggtctttccgctgcttttgtgcatcaccctcaacggactctgcgctctcggtcgcgagcacagctgataatagtccataaagtaggaaacactacagctggagcctcggtggccatgcttcttgccccttgggcccaaacaccattgtcacactccatccccccagcccctcctccccttcctgcacccgtacccccgtggtctttgaataaagtctgagtgggcggcgcggccgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 67 - protein, 5 aa, synthetic construct
  REGION 1..5: note = Synthetic: linker
GGGGS

SEQ ID NO: 68 - DNA, 302 bp, synthetic construct
  misc_feature 1..302: note = Synthetic: Alternative 3 UTR
  source 1..302: mol_type = other DNA
tgataatagtccataaagtaggaaacactacagctggagcctcggtggccatgcttcttgccccttgggcccaaacaccattgtcacactccatccccccagcccctcctccccttcctgcacccgtacccccgtggtctttgaataaagtctgagtgggcggcgcggccgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa